(19) United States
(12) Patent Application Publication
(10) Pub. No.: US 2025/0154145 A1
(43) Pub. Date: May 15, 2025

(54) OXER1 ANTAGONISTS AND USES THEREOF

(71) Applicant: Fairhaven Pharmaceuticals Ins., Laval (CA)

(72) Inventors: Shaun Abbott, Pointe Claire (CA); Jeremy Green, Laval (CA); Elyse Bourque, L'Étang-du-Nord (CA); Mylène De Léséleuc, Montreal (CA); Julien Martel, Laval (CA)

(21) Appl. No.: 18/719,534

(22) PCT Filed: Dec. 20, 2022
(86) PCT No.: PCT/CA2022/051864

§ 371 Date: Jun. 13, 2024

Related U.S. Application Data
(60) Provisional application No. 63/291,555, filed on Dec. 20, 2021.

(51) Int. Cl. C07D 471/04; A61K 31/381; A61K 31/404; A61K 31/405; A61K 31/41; A61K 31/4155; A61K 31/423; A61K 31/437; A61K 31/4439; A61K 31/444; A61K 31/454; A61K 31/4545; A61K 31/497; A61K 31/506; A61K 31/706; A61K 45/06; C07D 209/18; C07D 209/20; C07D 209/26; C07D 333/60; C07D 401/06; C07D 403/06; C07D 413/06; C07D 413/14; C07H 17/02

(52) U.S. Cl. CPC C07D 471/04; A61K 31/381; A61K 31/404; A61K 31/405; A61K 31/41; A61K 31/4155; A61K 31/423; A61K 31/437; A61K 31/4439; A61K 31/444; A61K 31/454; A61K 31/4545; A61K 31/497; A61K 31/506; A61K 31/706; A61K 45/06; C07D 209/18; C07D 209/20; C07D 209/26; C07D 333/60; C07D 401/06; C07D 403/06; C07D 413/06; C07D 413/14; C07H 17/02

(57) ABSTRACT

Disclosed are compounds of formula (I), wherein ring A is an 8-10 membered aromatic or partially aromatic bicyclic ring having 1-4 heteroatoms compositions thereof, wherein L1, L2, R1 to R4 and m are as defined in claim 1; and methods of using the same for antagonizing G-protein coupled receptor OXER1, and for treating of OXER1-mediated disorders, such as asthma or cancer.

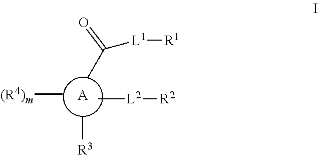

### CROSS-REFERENCE TO RELATED APPLICATIONS

This application claims the benefit of U.S. Provisional Application No. 63/291,555 filed on Dec. 20, 2021, the entirety of which is hereby incorporated by reference.

### TECHNICAL FIELD OF THE INVENTION

The present invention relates to compounds and methods useful for antagonizing G-protein coupled receptor OXER1. The invention also provides pharmaceutically acceptable compositions comprising compounds of the present invention and methods of using said compositions in the treatment of various disorders.

### BACKGROUND OF THE INVENTION

Arachidonic acid is a key biological intermediate that is converted to a large number of eicosanoids with potent biological activities. Metabolism of arachidonic acid by the 5-lipoxygenase (5-LO) pathway leads to the formation of leukotrienes such as LTB₄, LTC₄ and LTD₄, and 5S-hydroxy-6E,8Z,11Z,14Z-eicosatetraenoic acid (5-HETE). 5-HETE is oxidized to 5-oxo-6,8,11,14-eicosatetraenoic acid (5-oxo-ETE) by the action of 5-hydroxyeicosanoid dehydrogenase, a microsomal enzyme found in leukocytes and platelets, as well as endothelial and epithelial cells.

5-oxo-ETE is a potent chemoattractant for eosinophils and neutrophils, and elicits a variety of rapid responses in these cells. Examples of the responses in these cells in addition to cell migration and tissue infiltration include actin polymerization, calcium mobilization, integrin expression, shedding of L-selectin, degranulation, and superoxide production. The primary target of 5-oxo-ETE is most likely the eosinophil, and among lipid mediators it is the strongest chemoattractant for these cells. It has been shown to induce transendothelial migration of eosinophils and to induce the infiltration of both eosinophils and neutrophils into the skin. 5-oxo-ETE also promotes the survival of eosinophils and possibly other types of inflammatory cells through, for example, the induction of GM-CSF release from monocytes. 5-oxo-ETE is also a chemoattractant for monocytes and has been shown to stimulate the proliferation of prostate tumor cells. Migration of eosinophils to body sites including the skin, lungs and intestines is mediated by eosinophil chemo-attractants such as 5-oxo-ETE, and plays a key role in Type 2 inflammation-driven diseases, including skin, respiratory and gastro-intestinal diseases, such as, asthma, allergic rhinitis, chronic obstructive pulmonary disorder, atopic dermatitis, psoriasis, and acne. Migration of eosinophils has also been shown to play a role in certain cancers such as prostate cancer.

OXER1 is a G protein-coupled receptor (GPCR) that is highly selective for 5-oxo-ETE. It has also been termed G-protein coupled receptor 170 (GPR170), hGPCR48, HGPCR48, and R527. Interaction of 5-oxo-ETE with OXER1 is an important mediator of migration of eosinophils. Selective OXER1 antagonists may function as therapeutic or prophylactic agents for above mentioned diseases.

### SUMMARY OF THE INVENTION

It has now been found that compounds of this invention, and pharmaceutically acceptable compositions thereof, are effective as antagonists of OXER1. In certain embodiments, the invention provides for compounds of the formulae presented herein.

Compounds of the present invention, and pharmaceutically acceptable compositions thereof, are useful for treating a variety of diseases, disorders or conditions, associated with OXER1. Such diseases, disorders, or conditions include those described herein.

Compounds provided by this invention are also useful for the study of OXER1 in biological and pathological phenomena; the study of migration of eosinophils to body sites including the skin, lungs and intestines; and the comparative evaluation of new OXER1 inhibitors or other regulators of migration of eosinophils in vitro or in vivo.

### DETAILED DESCRIPTION OF CERTAIN EMBODIMENTS

### 1. General Description of Certain Embodiments of the Invention:

In certain aspects, the present invention provides a compound of formula I.

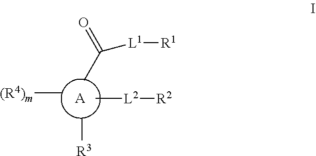

or an N-oxide, or a pharmaceutically acceptable salt thereof, wherein each of Ring A, L¹, L², R¹, R², R³, R⁴, and m, is as defined below and described in embodiments herein, both singly and in combination.

In some embodiments, the present invention provides a pharmaceutical composition comprising a compound of formula I and a pharmaceutically acceptable carrier, adjuvant, or diluent.

In some embodiments, the present invention provides a method of treating a OXER1-mediated disease, disorder, or condition comprising administering to a patient in need thereof, a compound of formula I, or an N-oxide, or a pharmaceutically acceptable salt thereof.

### 2. Compounds and Definitions:

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

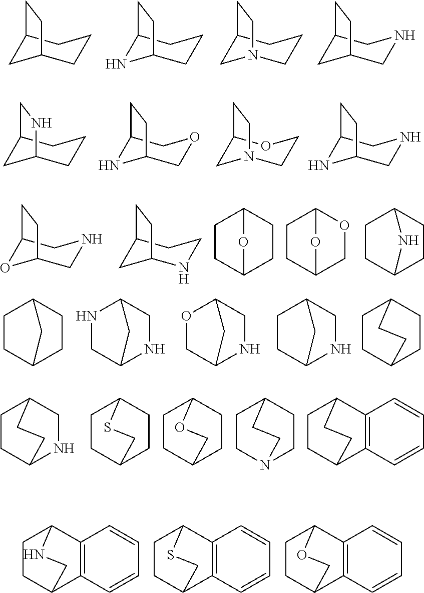

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “oxo” refers to a “═O” substituent. For example, a cyclopentane substituted with an oxo group is cyclopentanone

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

A partially aromatic bicyclic ring refers to a bicyclic ring in which one ring of the bicyclic ring is aromatic and the other ring of the bicyclic ring is not aromatic.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —N(R^{∘})C(NR^{∘})N(R^{∘})₂; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

In certain embodiments, R¹ of a provided compound comprises one or more deuterium atoms. In certain embodiments, R² of a provided compound comprises one or more deuterium atoms. In certain embodiments, R³ of a provided compound comprises one or more deuterium atoms. In certain embodiments, R⁴ of a provided compound comprises one or more deuterium atoms. In certain embodiments, R⁵ of a provided compound comprises one or more deuterium atoms. In certain embodiments, L¹ of a provided compound comprises one or more deuterium atoms. In certain embodiments, L² of a provided compound comprises one or more deuterium atoms. In certain embodiments, L³ of a provided compound comprises one or more deuterium atoms. In certain embodiments, Ring A of a provided compound comprises one or more deuterium atoms. In certain embodiments, Ring B of a provided compound comprises one or more deuterium atoms. In certain embodiments, Ring C of a provided compound comprises one or more deuterium atoms. In certain embodiments, R of a provided compound may be substituted with one or more deuterium atoms. In certain embodiments, R^{z} of a provided compound may be substituted with one or more deuterium atoms.

The structures as drawn represent relative configurations, unless labeled as absolute configurations. The invention contemplates individual enantiomers and racemic mixtures. Also contemplated are diastereomeric mixtures.

As used herein, a “OXER1 antagonist” or a “OXER1 inhibitor” is a molecule that reduces, inhibits, or otherwise diminishes one or more of the biological activities of OXER1 (e.g. Gαi signaling, increased immune cell migration, and secretion of proinflammatory cytokines). Antagonism using the OXER1 antagonist does not necessarily indicate a total elimination of the OXER1 activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of OXER1 compared to an appropriate control. In some embodiments, the OXER1 antagonist reduces, inhibits, or otherwise diminishes the activity of OXER1. The presently disclosed compounds bind directly to OXER1 and inhibit its activity.

By “specific antagonist” is intended an agent that reduces, inhibits, or otherwise diminishes the activity of a defined target greater than that of an unrelated target. For example, a OXER1 specific antagonist reduces at least one biological activity of OXER1 by an amount that is statistically greater than the inhibitory effect of the antagonist on any other protein (e.g., other GPCRs). In some embodiments, the IC₅₀ of the antagonist for the target is about 90%, 80%, 70%, 60%, 50%, 40%, 30%, 20%, 10%, 5%, 1%, 0.1%, 0.01%, 0.001% or less of the IC₅₀ of the antagonist for a non-target. The presently disclosed compounds may or may not be a specific OXER1 antagonist. A specific OXER1 antagonist reduces the biological activity of OXER1 by an amount that is statistically greater than the inhibitory effect of the antagonist on any other protein (e.g., other GPCRs). In certain embodiments, the OXER1 antagonist specifically inhibits the activity of OXER1. In some of these embodiments, the IC₅₀ of the OXER1 antagonist for OXER1 is about 90%, 80%, 70%, 60%, 50%, 40%, 30%, 20%, 10%, 0.1%, 0.01%, 0.001%, or less of the IC₅₀ of the OXER1 antagonist for a closely related GPCR (e.g. a free fatty acid receptor (FFAR) such as GPR40 (FFAR1), GPR41 (FFAR3), GPR43 (FFAR2), or GPR120 (FFAR4)) or other type of GPCR (e.g., a Class A GPCR).

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57. In some embodiments, such moieties may be attached via a strained alkyne. Methods of using strained alkynes to enable rapid Cu-free click chemistry are known in the art and include those described by Jewett et al., J. Am. Chem. Soc. 2010, 132(11), 3688-3690.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

A compound of the present invention may be tethered to an E3 ligase binding moiety. It will be appreciated that such compounds are useful as degraders (see, for example, Kostic and Jones, Trends Pharmacol. Sci., 2020, 41(5), 305-31; Ottis and Crews, ACS Chem. Biol. 2017, 12(4), 892-898). One of ordinary skill in the art will recognize that an E3 ligase binding moiety may be attached to a provided compound via a suitable substituent as defined above. Such degraders have been found to be useful for the targeted degradation of G-protein coupled receptors (Li et al. Acta Pharm. Sin. B. 2020, 10(9), 1669-1679).

As used herein, the term “E3 ligase binding moiety” is used interchangeably with the term “E3 ligase binder” and relates to any moiety capable of binding to and/or recruiting an E3 ligase (e.g., cIAP1, MDM2, cereblon, VHL, APC/C) for targeted degradation.

A compound of the present invention may be tethered to a lysosome targeting moiety. It will be appreciated that such compounds are useful as degraders (see, for example, Banik et al. 2020. Nature 584, 291-297). One of ordinary skill in the art will recognize that a lysosome targeting moiety may be attached to a provided compound via a suitable substituent as defined above. Such degraders have been found to be useful for the targeted degradation of secreted and membrane proteins (Banik et al. 2020).

As used herein, the term “lysosome targeting moiety” is used interchangeably with the term “lysosome binding moiety” and relates to any moiety capable of binding to and/or recruiting a cell surface lysosome targeting receptor (e.g., cation-independent mannose-6-phosphate receptor, CI-M6PR) for targeted degradation.

The terms “measurable affinity” and “measurably inhibit,” as used herein, means a measurable change in a OXER1 activity between a sample comprising a compound of the present invention, or composition thereof, and a OXER1 GPCR, and an equivalent sample comprising a OXER1 GPCR, in the absence of said compound, or composition thereof.

The terms “a” and “an” as used herein mean “one or more” and include the plural unless the context is inappropriate.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. 3. Description of Exemplary Embodiments:

As described above, in certain embodiments, the present invention provides a compound of formula I:

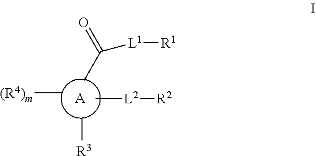

or an N-oxide, or a pharmaceutically acceptable salt thereof, wherein:
Ring A is an 8-10 membered aromatic or partially aromatic bicyclic ring having 1-4 heteroatoms independently selected from nitrogen, oxygen, and sulfur; a 5-6 membered monocyclic heteroaryl ring having 1-4 heteroatoms independently selected from nitrogen, oxygen, and sulfur; or phenyl;
L¹ is one of the following:
   (a) a C₁₋₅ bivalent straight or branched, saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —C(F)₂—, —N(R)—, —S—, —S(O)—, or —S(O)₂—; or
   (b) a covalent bond;
each R is independently hydrogen, or an optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur;
each -Cy- is independently an optionally substituted bivalent ring selected from a 4-7 membered saturated or partially unsaturated heterocyclylenyl having 1-2 heteroatoms independently selected from nitrogen, oxygen, and sulfur, phenylenyl, a 3-7 membered saturated or partially unsaturated carbocyclylenyl, or a 5-6 membered heteroarylenyl having 1-4 heteroatoms independently selected from nitrogen, oxygen, and sulfur;
L² is one of the following:
   (a) a C₁₋₇ bivalent straight or branched saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —CH(F)—, —C(F)₂—, —N(R)—, —S—, —S(O)—, or —S(O)₂—; or
   (b) a covalent bond;
R¹ is selected from (i) —C(O)OR, —C(O)N(R)S(O)₂R, —C(O)N(R)OR, —C(O)NR₂, —CN, —OH, and hydrogen; (ii) a 5-6 membered partially unsaturated oxo-heterocyclyl containing 1-4 heteroatoms independently selected from nitrogen, oxygen, and sulfur; which is substituted with n instances of R⁵; and (iii) a 5-6 membered monocyclic heteroaryl ring having 1-4 heteroatoms independently selected from nitrogen, oxygen, and sulfur; which is substituted with n instances of R⁵;
R² is one of the following:
   (a) phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered aromatic or partially aromatic bicyclic ring having 1-4 heteroatoms independently selected from nitrogen, oxygen, and sulfur; each of which is substituted with p instances of R⁶; or
   (b) C₁₋₇ aliphatic, —C≡CR, —C(O)OR, or —C(O)R; each of which is substituted with p instances of R⁶; or
   (c) hydrogen;
R³ is one of the following:
   (a) a C₁₋₆ aliphatic group optionally substituted with one or more —OH or —N(R)₂;
   (b) —CD₃;
   (c) hydrogen; or
   (d) absent;
each instance of R⁴ is independently hydrogen, deuterium, R^{z}, —C≡CR, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂; or
   two R⁴ groups are optionally taken together to form ═O; or
   two R⁴ groups are optionally taken together with their intervening atoms to form an optionally substituted 5-8 membered saturated, partially unsaturated, or aryl fused ring having 0-3 heteroatoms independently selected from nitrogen, oxygen and sulfur;
each instance of R⁵, and R⁶ is independently hydrogen, deuterium, R^{z}, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂; or
   two R⁵ groups are optionally taken together to form ═O;
   two R⁶ groups are optionally taken together to form ═O;
   two R⁵ groups are optionally taken together with their intervening atoms to form an optionally substituted 5-8 membered saturated, partially unsaturated, or aryl fused ring having 0-3 heteroatoms independently selected from nitrogen, oxygen and sulfur; or
   two R⁶ groups are optionally taken together with their intervening atoms to form an optionally substituted 5-8 membered saturated, partially unsaturated, or aryl fused ring having 0-3 heteroatoms independently selected from nitrogen, oxygen and sulfur;
each instance of R^{z} is independently selected from an optionally substituted group selected from C₁₋₆ aliphatic; phenyl; a 4-7 membered saturated or partially unsaturated heterocyclic ring having 1-2 heteroatoms independently selected from nitrogen, oxygen, and sulfur; and a 5-6 membered heteroaryl ring having 1-4 heteroatoms independently selected from nitrogen, oxygen, and sulfur;
m is 0, 1, 2, 3, or 4;
n is 0, 1, 2, 3, or 4; and
p is 0, 1, 2, 3, or 4;
provided that if L¹-R¹ is

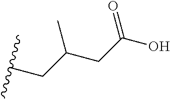 [SMILES: *CC(C)CC(=O)O]

and ring A with its R³ and R⁴ substituents is

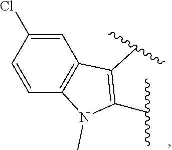 [SMILES: *c1c(*)n(C)c2ccc(Cl)cc12]

then L²-R² is not n-hexyl,

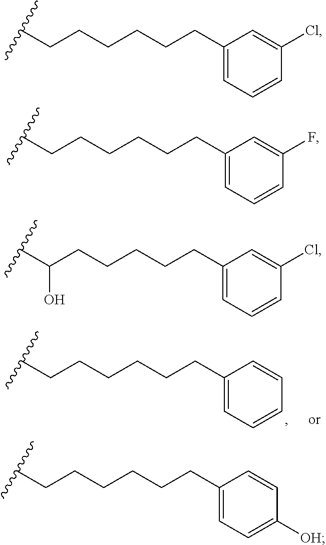

and provided that if L¹-R¹ is

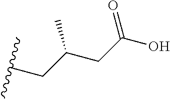 [SMILES: *C[C@H](C)CC(=O)O]

and ring A with its R³ and R⁴ substituents is

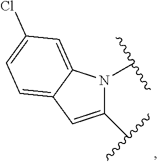 [SMILES: *c1cc2ccc(Cl)cc2n1*]

then L²-R² is not n-hexyl.

As defined generally above, Ring A is an 8-10 membered aromatic or partially aromatic bicyclic ring having 1-4 heteroatoms independently selected from nitrogen, oxygen, and sulfur; a 5-6 membered monocyclic heteroaryl ring having 1-4 heteroatoms independently selected from nitrogen, oxygen, and sulfur; or phenyl.

In some embodiments, Ring A is an 8-10 membered aromatic or partially aromatic bicyclic ring having 1-4 heteroatoms independently selected from nitrogen, oxygen, and sulfur. In some embodiments, Ring A is a 5-6 membered monocyclic heteroaryl ring having 1-4 heteroatoms independently selected from nitrogen, oxygen, and sulfur. In some embodiments, Ring A is phenyl.

In some embodiments, Ring A substituted with R⁴, R³, —C(O)-L¹-R¹, and -L²-R² is

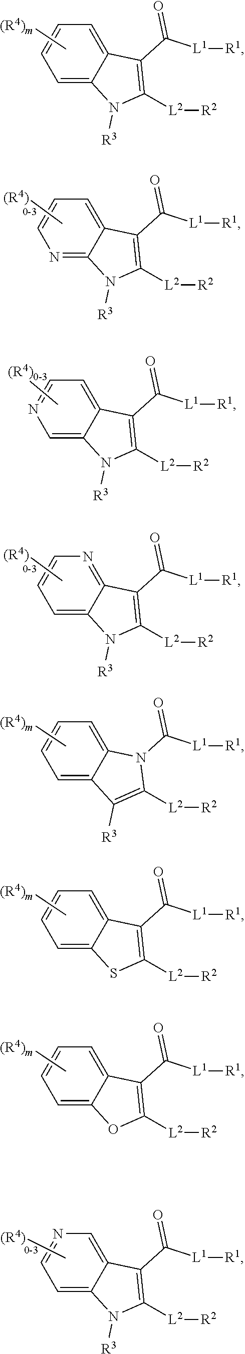

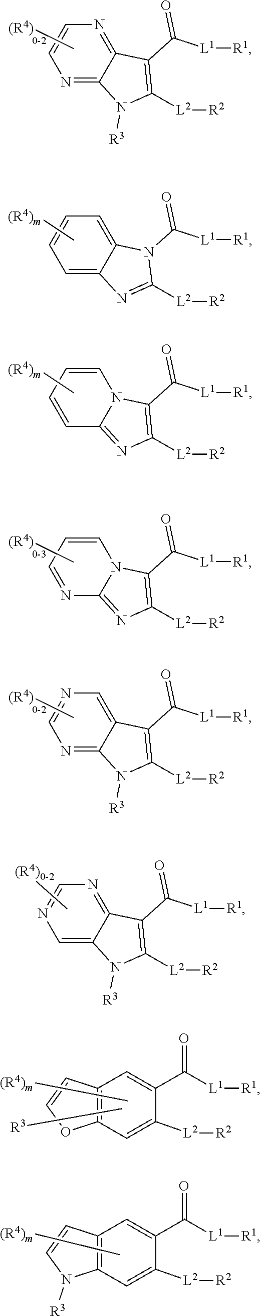

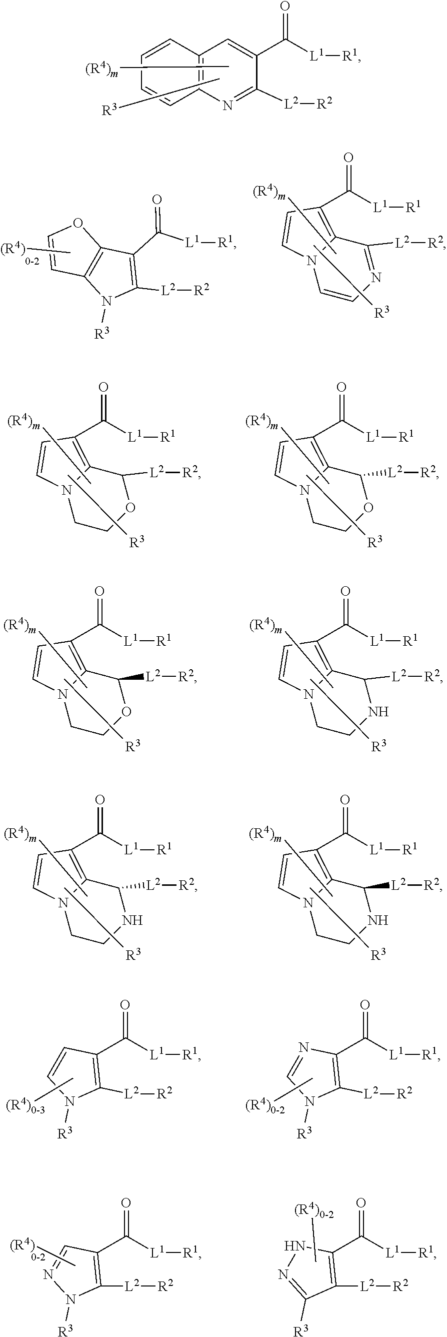

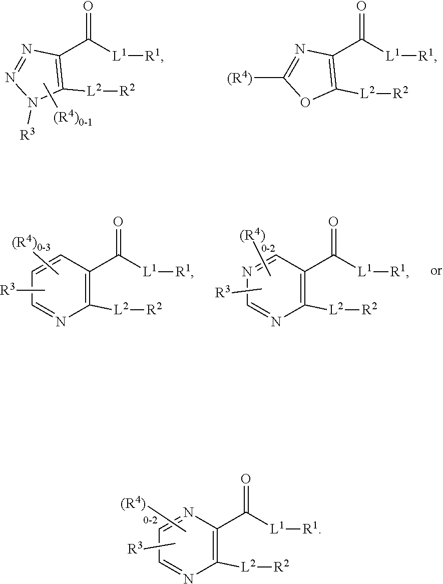

In some embodiments, Ring A is selected from those depicted in Table 1, below.

As defined generally above, L¹ is one of the following: (a) C₁₋₅ bivalent straight or branched, saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —C(F)₂—, —N(R)—, —S—, —S(O)—, or —S(O)₂—; or (b) a covalent bond.

In some embodiments, L¹ is C₁₋₅ bivalent straight or branched, saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —C(F)₂—, —N(R)—, —S—, —S(O)—, or —S(O)₂—.

In some embodiments, L¹ is a C₁₋₅ bivalent straight or branched, saturated hydrocarbon chain. In some embodiments, L¹ is

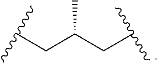

In some embodiments, L¹ is a C₁₋₅ bivalent straight or branched, saturated hydrocarbon chain wherein 1 methylene unit of the chain is replaced with -Cy-, wherein Cy is a bivalent ring selected from a 4-7 membered saturated heterocyclylenyl having 1-2 heteroatoms independently selected from nitrogen, oxygen, and sulfur. In some embodiments, L¹ is

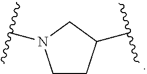 [SMILES: *C1CCN(*)C1]

In some embodiments, L¹ is a covalent bond.

In some embodiments L¹ is

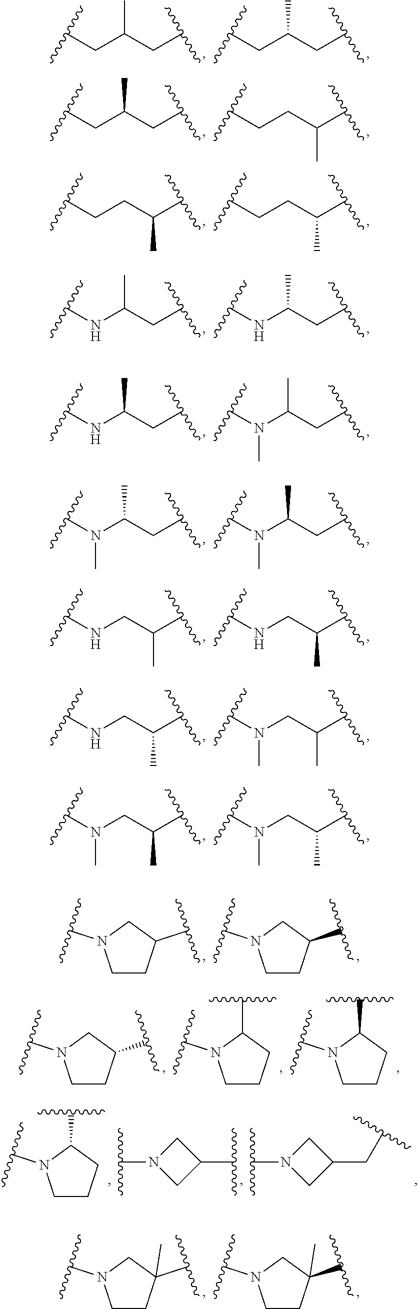

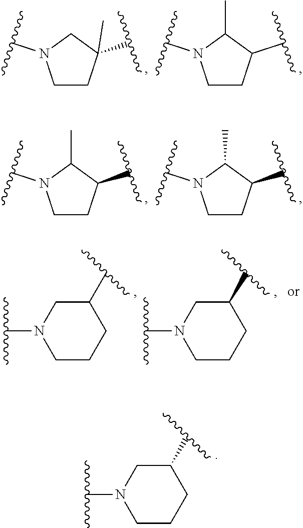

In some embodiments L¹ is

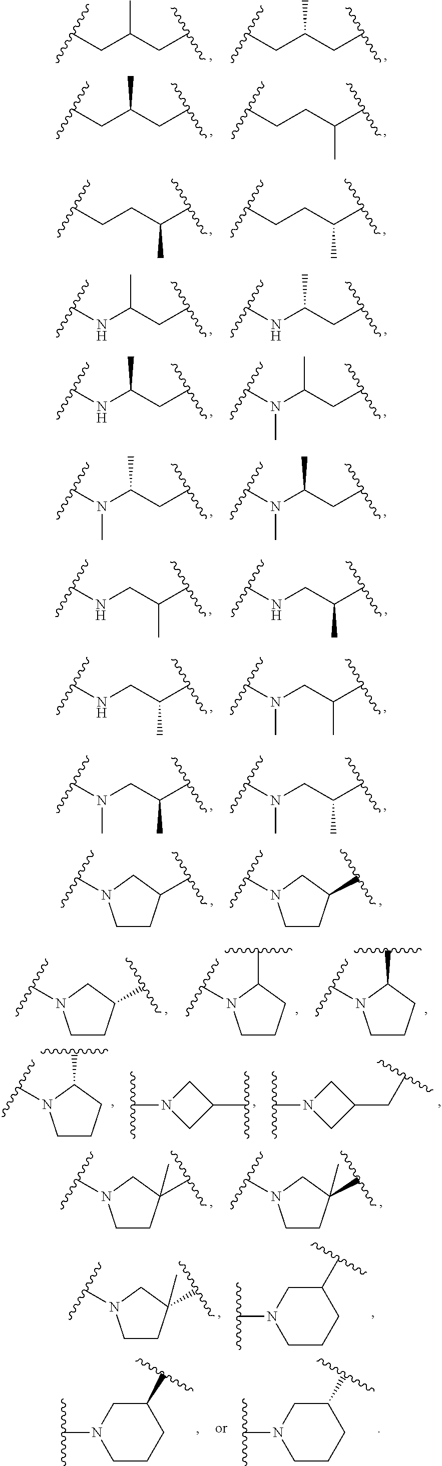

In some embodiments, L¹ is selected from those depicted in Table 1, below.

As defined generally above, each R is independently hydrogen, or an optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur.

In some embodiments, R is hydrogen. In some embodiments, R is an optionally substituted group selected from C₁₋₆ aliphatic. In some embodiments, R is phenyl. In some embodiments, R is an 8-10 membered bicyclic aryl ring, a 3-7 membered saturated or partially unsaturated monocyclic carbocyclic ring. In some embodiments, R is a 4-8 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur. In some embodiments, R is an 8-10 membered bicyclic heteroaryl ring having 1-5 heteroatoms independently selected from nitrogen, oxygen, and sulfur.

As defined generally above, two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur.

In some embodiments, two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur.

In some embodiments, R is selected from those depicted in Table 1, below.

As defined generally above, each -Cy- is independently an optionally substituted bivalent ring selected from a 4-7 membered saturated or partially unsaturated heterocyclylenyl having 1-2 heteroatoms independently selected from nitrogen, oxygen, and sulfur, phenylenyl, a 3-7 membered saturated or partially unsaturated carbocyclylenyl, or a 5-6 membered heteroarylenyl having 1-4 heteroatoms independently selected from nitrogen, oxygen, and sulfur.

In some embodiments, -Cy- is an optionally substituted bivalent ring selected from a 4-7 membered saturated or partially unsaturated heterocyclylenyl having 1-2 heteroatoms independently selected from nitrogen, oxygen, and sulfur.

In some embodiments, -Cy- is phenylenyl.

In some embodiments, -Cy- is a 3-7 membered saturated or partially unsaturated carbocyclylenyl, or a 5-6 membered heteroarylenyl having 1-4 heteroatoms independently selected from nitrogen, oxygen, and sulfur.

In some embodiments, -Cy- is selected from those depicted in Table 1, below.

As defined generally above, L² is one of the following: (a) a C₁₋₇ bivalent straight or branched saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —CH(F)—, —C(F)₂—, —N(R)—, —S—, —S(O)—, or —S(O)₂—; or (b) a covalent bond.

In some embodiments, L² is a C₁₋₇ bivalent straight or branched saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —CH(F)—, —C(F)₂—, —N(R)—, —S—, —S(O)—, or —S(O)₂—.

In some embodiments, L² is a C₁₋₇ bivalent straight or branched saturated hydrocarbon chain wherein 1 methylene unit of the chain is replaced with —O—. In some embodiments, L² is —(CH₂)₃₋₄—O—(CH₂)₁₋₂—. In some embodiments, L² is —(CH₂)₃₋₄—O—(C(H)(CH₃))—. In some embodiments, L² is a C₁₋₇ bivalent straight or branched unsaturated hydrocarbon chain. In some embodiments, L² is a C₁ bivalent straight unsaturated hydrocarbon chain.

In some embodiments, L² is a covalent bond.

In some embodiments, L² is

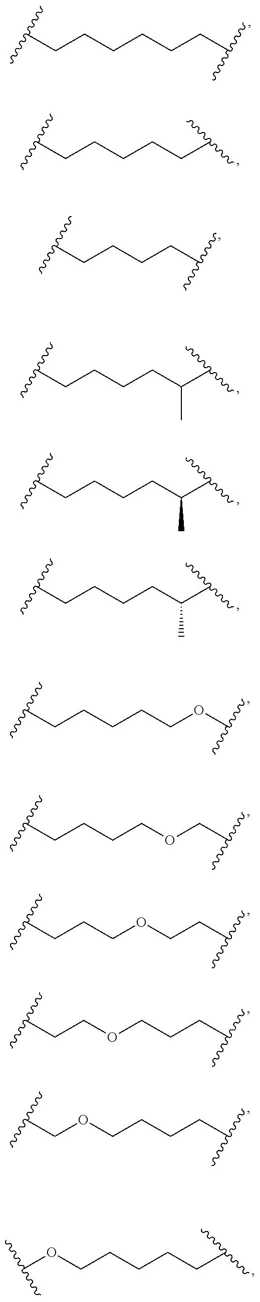

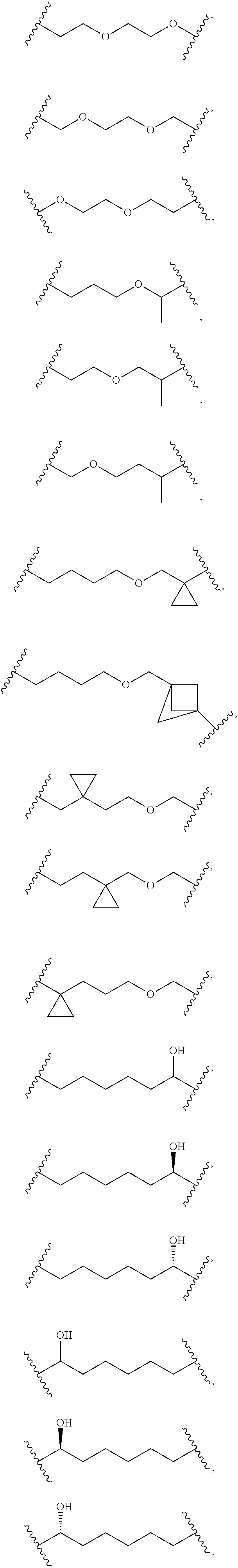

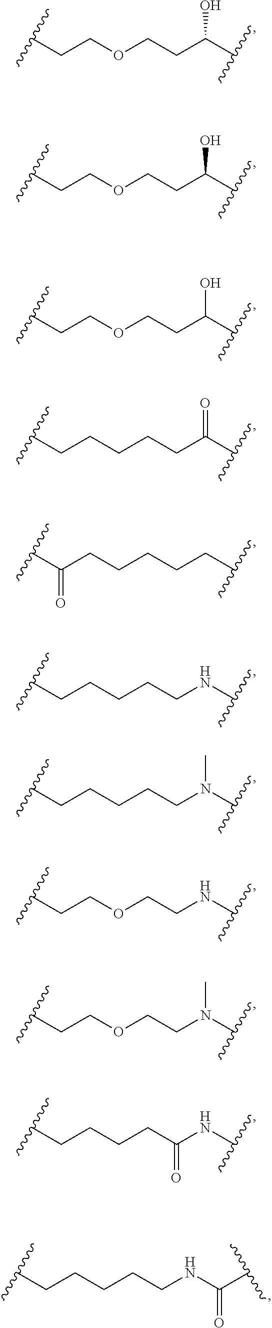

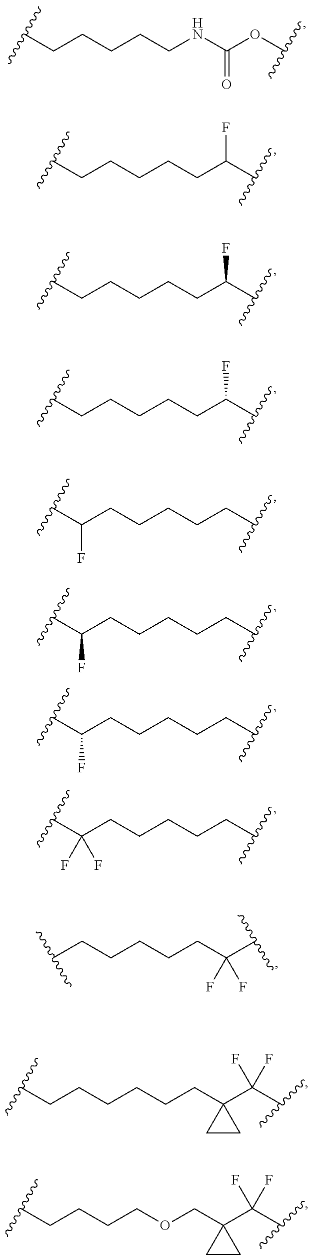

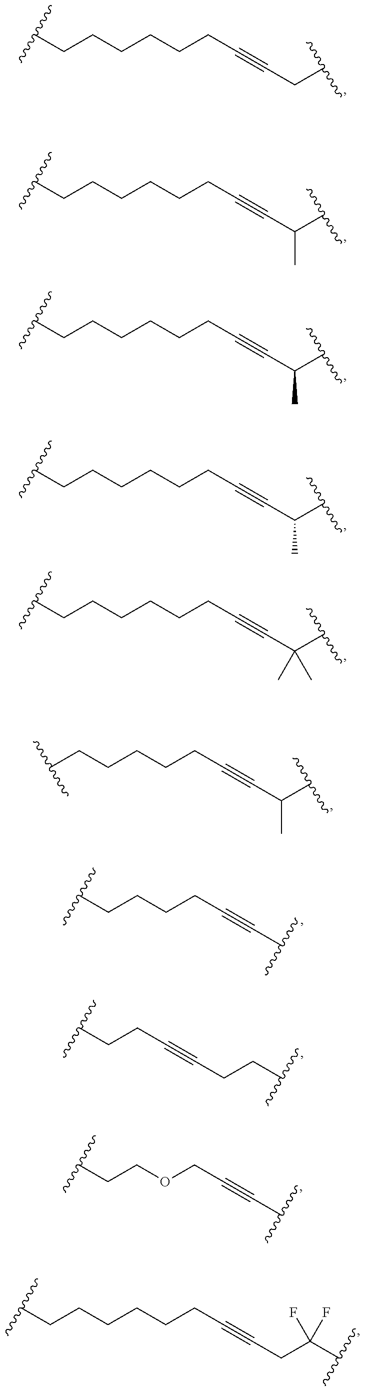

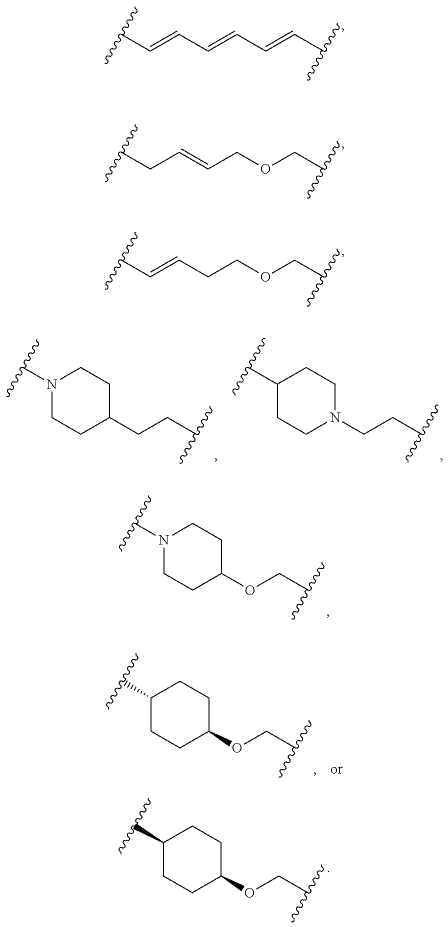

In some embodiments, L² is selected from those depicted in Table 1, below.

As defined generally above, R¹ is selected from (i) —C(O)OR, —C(O)N(R)S(O)₂R, —C(O)N(R)OR, —C(O)NR₂, —CN, —OH, and hydrogen; (ii) a 5-6 membered partially unsaturated oxo-heterocyclyl containing 1-4 heteroatoms independently selected from nitrogen, oxygen, and sulfur; which is substituted with n instances of R⁵; and (iii) a 5-6 membered monocyclic heteroaryl ring having 1-4 heteroatoms independently selected from nitrogen, oxygen, and sulfur; which is substituted with n instances of R⁵.

In some embodiments, R1 is selected from (i) —C(O)OR, —C(O)N(R)S(O)₂R, —C(O)N(R)OR, —C(O)NR₂, —CN, —OH, and hydrogen. In some embodiments, R¹ is a 5-6 membered partially unsaturated oxo-heterocyclyl containing 1-4 heteroatoms independently selected from nitrogen, oxygen, and sulfur; which is substituted with n instances of R⁵. In some embodiments, R¹ is a 5-6 membered monocyclic heteroaryl ring having 1-4 heteroatoms independently selected from nitrogen, oxygen, and sulfur; which is substituted with n instances of R⁵.

In some embodiments, R¹ is selected from (i) —C(O)OR, —C(O)N(R)S(O)₂R, and —C(O)N(R)OR; and (ii) a 5-6 membered monocyclic heteroaryl ring having 1-4 heteroatoms independently selected from nitrogen, oxygen, and sulfur; which is substituted with n instances of R⁵.

In some embodiments, R¹ is —C(O)OR. In some embodiments, R¹ is —C(O)N(R)S(O)₂R. In some embodiments, R¹ is —C(O)N(R)OR. In some embodiments, R¹ is a 5-6 membered monocyclic heteroaryl ring having 1-4 heteroatoms independently selected from nitrogen, oxygen, and sulfur; which is substituted with n instances of R⁵. In some embodiments, R¹ is

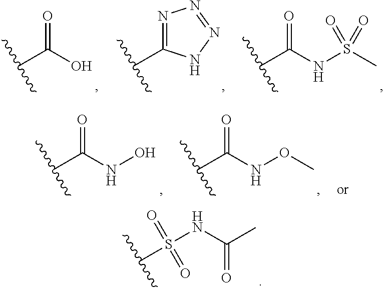

In some embodiments, R¹ is —C(O)NR₂. In some embodiments, R¹ is

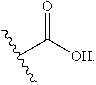 [SMILES: *C(=O)O]

In some embodiments, R¹ is —CN. In some embodiments, R¹ is —OH. In some embodiments, R¹ is hydrogen.

In some embodiments, R¹ is selected from those depicted in Table 1, below.

As defined generally above, R² is one of the following:
(a) phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered aromatic or partially aromatic bicyclic ring having 1-4 heteroatoms independently selected from nitrogen, oxygen, and sulfur; each of which is substituted with p instances of R⁶; or
(b) C₁₋₇ aliphatic, —C≡CR, —C(O)OR, or —C(O)R; each of which is substituted with p instances of R⁶; or
(c) hydrogen.

In some embodiments, R² is phenyl, which is substituted with p instances of R⁶. In some embodiments, R² is a 5-6 membered monocyclic heteroaryl ring having 1-4 heteroatoms independently selected from nitrogen, oxygen, and sulfur; which is substituted with p instances of R⁶. In some embodiments, R² is a 3-7 membered saturated or partially unsaturated monocyclic carbocyclic ring; which is substituted with p instances of R⁶. In some embodiments, R² is a 4-8 membered saturated or partially unsaturated monocyclic heterocyclic ring having 1-2 heteroatoms independently selected from nitrogen, oxygen, and sulfur; which is substituted with p instances of R⁶. In some embodiments, R² is a 5-10 membered saturated or partially unsaturated bridged bicyclic ring having 0-3 heteroatoms independently selected from nitrogen, oxygen, and sulfur, which is substituted with p instances of R⁶. In some embodiments, R² is a 5-10 membered saturated or partially unsaturated spirocyclic ring having 0-3 heteroatoms independently selected from nitrogen, oxygen, and sulfur. which is substituted with p instances of R⁶. In some embodiments, R² is or an 8-10 membered aromatic or partially aromatic bicyclic ring having 1-4 heteroatoms independently selected from nitrogen, oxygen, and sulfur; which is substituted with p instances of R⁶. In some embodiments, R² is C₁₋₇ aliphatic, which is substituted with p instances of R⁶. In some embodiments, R² is —C≡CR. In some embodiments, R² is —C(O)OR. In some embodiments, R² is —C(O)R. In some embodiments, R² is hydrogen.

In some embodiments, R² is

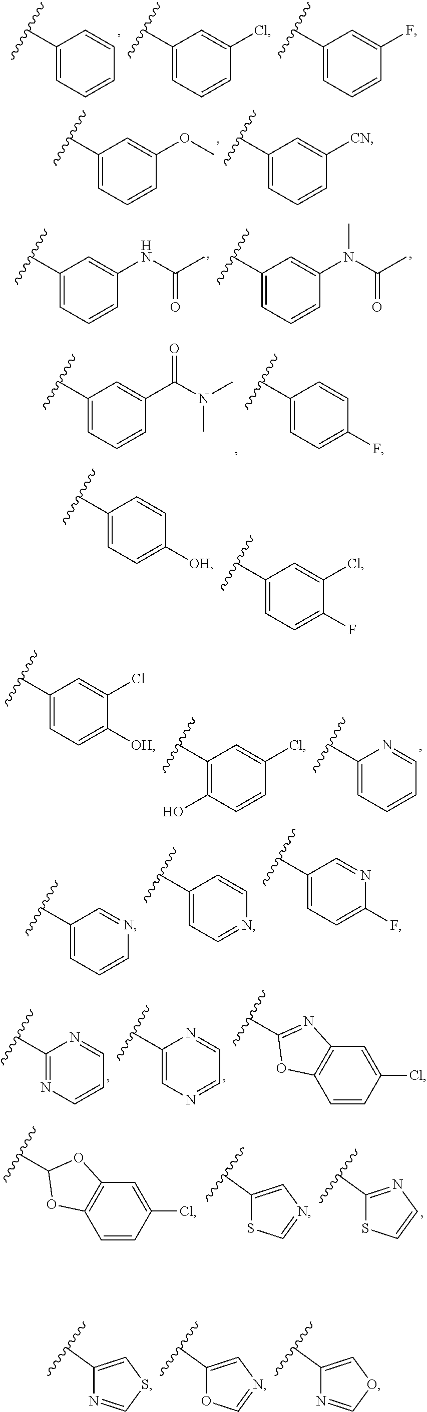

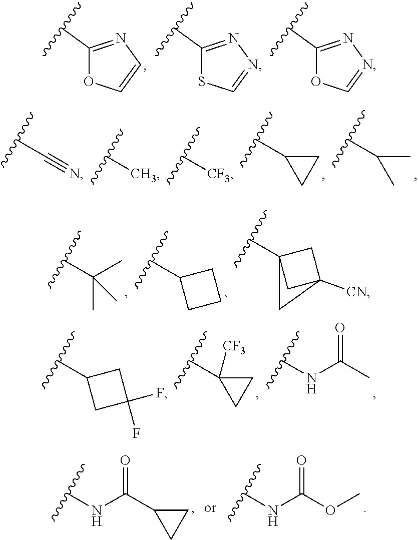

In some embodiments, R² is

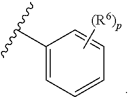

In some embodiments, R² is —C≡CR. In some embodiments, R² is —C≡C—(3-7 membered saturated monocyclic carbocylic ring substituted with 1 or 2 halo groups). In some embodiments, R² is —C≡C—(cyclobutyl substituted with 1 or 2 halo groups).

In some embodiments, R² is selected from those depicted in Table 1, below.

As defined generally above, R³ is one of the following: (a) a C₁₋₆ aliphatic group optionally substituted with one or more —OH or —N(R)₂; (b) —CD₃; (c) hydrogen; or (d) absent.

In some embodiments, R³ is a C₁₋₆ aliphatic group optionally substituted with one or more —OH or —N(R)₂. In some embodiments, R³ is —CH₃. In some embodiments, R³ is —CD₃. In some embodiments, R³ is hydrogen. In some embodiments, R³ is OH

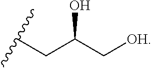 [SMILES: *C[C@@H](O)CO]

In some embodiments, R³ is absent.

In some embodiments, R³ is selected from those depicted in Table 1, below.

As defined generally above, each instance of R⁴ is independently hydrogen, deuterium, R^{z}, —C≡CR, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂.

In some embodiments, R⁴ is hydrogen, deuterium, R^{z}, —C≡CR, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂.

In some embodiments, R⁴ is hydrogen. In some embodiments, R⁴ is —Cl. In some embodiments, R⁴ is —CF₃. In some embodiments, R⁴ is —OCH₃. In some embodiments, R⁴ is —OCF₃. In some embodiments, R⁴ is —CN. In some embodiments, R⁴ is

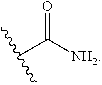 [SMILES: *C(N)=O]

As defined generally above, two R⁴ groups are optionally taken together to form ═O.

In some embodiments, two R⁴ groups are optionally taken together to form ═O.

As defined generally above, two R⁴ groups are optionally taken together with their intervening atoms to form an optionally substituted 5-8 membered saturated, partially unsaturated, or aryl fused ring having 0-3 heteroatoms independently selected from nitrogen, oxygen and sulfur.

In some embodiments, two R⁴ groups are optionally taken together with their intervening atoms to form an optionally substituted 5-8 membered saturated, partially unsaturated, or aryl fused ring having 0-3 heteroatoms independently selected from nitrogen, oxygen and sulfur.

In some embodiments, R⁴ is selected from those depicted in Table 1, below.

As defined generally above, each instance of R⁵, and R⁶ is independently hydrogen, deuterium, R^{z}, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂.

In some embodiments, R⁵ is hydrogen, deuterium, R^{z}, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂.

As defined generally above, two R⁵ groups are optionally taken together to form ═O.

In some embodiments, two R⁵ groups are optionally taken together to form ═O.

As defined generally above, two R⁵ groups are optionally taken together with their intervening atoms to form an optionally substituted 5-8 membered saturated, partially unsaturated, or aryl fused ring having 0-3 heteroatoms independently selected from nitrogen, oxygen and sulfur.

In some embodiments, two R⁵ groups are optionally taken together with their intervening atoms to form an optionally substituted 5-8 membered saturated, partially unsaturated, or aryl fused ring having 0-3 heteroatoms independently selected from nitrogen, oxygen and sulfur.

In some embodiments, R⁵ is selected from those depicted in Table 1, below.

In some embodiments, R⁶ is hydrogen, deuterium, R^{z}, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂.

As defined generally above, two R⁶ groups are optionally taken together to form ═O.

In some embodiments, two R⁶ groups are optionally taken together to form ═O.

As defined generally above, two R⁶ groups are optionally taken together with their intervening atoms to form an optionally substituted 5-8 membered saturated, partially unsaturated, or aryl fused ring having 0-3 heteroatoms independently selected from nitrogen, oxygen and sulfur.

In some embodiments, R⁴ and R⁶ each represent independently for each occurrence hydrogen, halogen, or C₁₋₆ aliphatic

In some embodiments, two R⁶ groups are optionally taken together with their intervening atoms to form an optionally substituted 5-8 membered saturated, partially unsaturated, or aryl fused ring having 0-3 heteroatoms independently selected from nitrogen, oxygen and sulfur.

In some embodiments, R⁶ is selected from those depicted in Table 1, below.

As defined generally above, each instance of R^{z} is independently selected from an optionally substituted group selected from C₁₋₆ aliphatic; phenyl; a 4-7 membered saturated or partially unsaturated heterocyclic ring having 1-2 heteroatoms independently selected from nitrogen, oxygen, and sulfur; and a 5-6 membered heteroaryl ring having 1-4 heteroatoms independently selected from nitrogen, oxygen, and sulfur.

In some embodiments, R^{z} is independently selected from an optionally substituted group selected from C₁₋₆ aliphatic; phenyl; a 4-7 membered saturated or partially unsaturated heterocyclic ring having 1-2 heteroatoms independently selected from nitrogen, oxygen, and sulfur; and a 5-6 membered heteroaryl ring having 1-4 heteroatoms independently selected from nitrogen, oxygen, and sulfur.

In some embodiments, R^{z} is selected from those depicted in Table 1, below.

As defined generally above, m is 0, 1, 2, 3, or 4.

In some embodiments, m is 0, 1, 2, 3, or 4. In some embodiments, m is 0. In some embodiments, m is 1. In some embodiments, m is 2. In some embodiments, m is 3. In some embodiments, m is 4.

In some embodiments, m is selected from those depicted in Table 1, below.

As defined generally above, n is 0, 1, 2, 3, or 4.

In some embodiments, n is 0, 1, 2, 3, or 4. In some embodiments, n is 0. In some embodiments, n is 1. In some embodiments, n is 2. In some embodiments, n is 3. In some embodiments, n is 4.

In some embodiments, n is selected from those depicted in Table 1, below.

As defined generally above, p is 0, 1, 2, 3, or 4.

In some embodiments, p is 0, 1, 2, 3, or 4. In some embodiments, p is 0. In some embodiments, p is 1. In some embodiments, p is 2. In some embodiments, p is 3. In some embodiments, p is 4.

In some embodiments, p is selected from those depicted in Table 1, below.

In some embodiments, if L¹-R¹ is

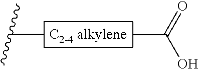

and ring A with its R³ and R⁴ substituents is

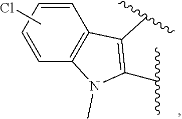

then L²-R² is not C₃₋₆ alkyl,

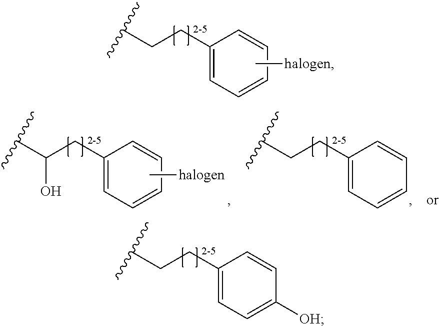

and provided that if L¹-R¹ is

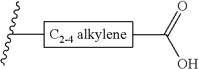

and ring A with its R³ and R⁴ substituents is

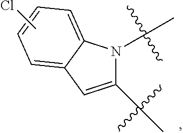

then L²-R² is not C₃₋₆ alkyl.

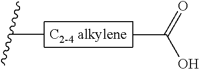

In some embodiments, if L¹-R¹ is a OH and ring A with its R³ and R⁴ substituents is a

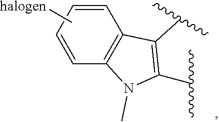

then L²-R² is not a C₃₋₈ alkyl,

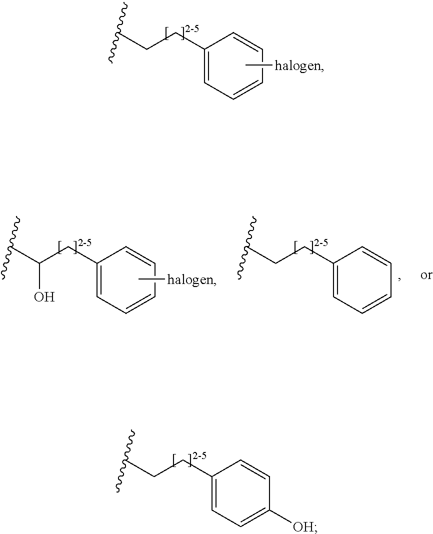

and provided that if L¹-R¹ is a

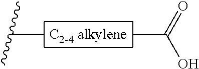

and ring A with its R³ and R⁴ substituents is a

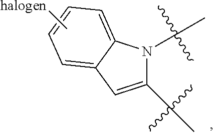

then L²-R² is not a C₃₋₈ alkyl.

In some embodiments, the present invention provides a compound of formula I, wherein Ring A is indolyl, azaindolyl, benzothiophenyl, or benzofuranyl, to provide a compound of formula I-a-1, I-a-2, I-a-3, or I-a-4:

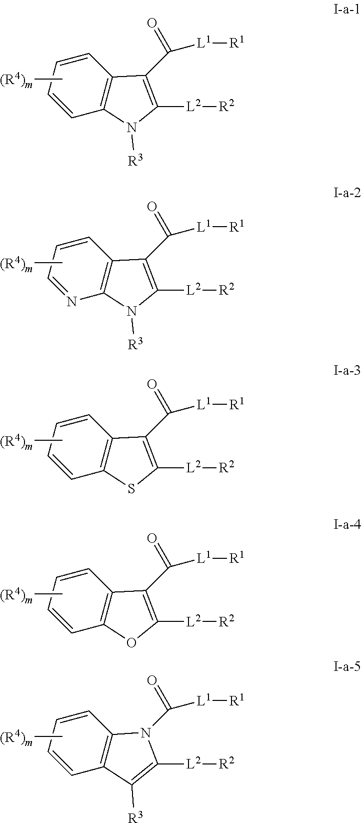

or an N-oxide, or a pharmaceutically acceptable salt thereof, wherein each of L¹, L², R¹, R², R³, R⁴, and m is as defined above and described in embodiments herein, both singly and in combination.

In some embodiments, the present invention provides a compound of formula I, wherein L¹ is 2-methylpropylene, to provide a compound of formula I-b-1, I-b-2, or I-b-3:

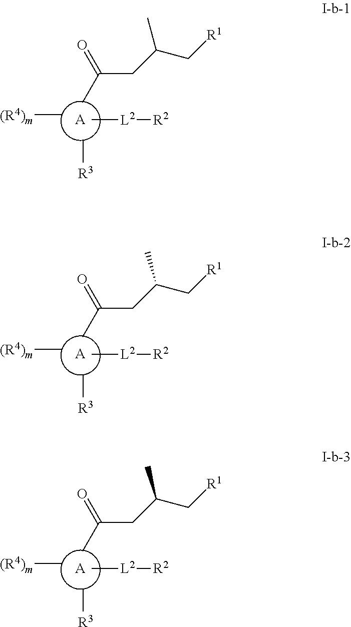

or an N-oxide, or a pharmaceutically acceptable salt thereof, wherein each of Ring A, L², R¹, R², R³, R⁴, and m is as defined above and described in embodiments herein, both singly and in combination.

In some embodiments, the present invention provides a compound of formula I, wherein L¹ is pyrrolidinyl, piperidinyl, or azetidinyl, to provide a compound of formula I-b-4, I-b-5, or I-b-6:

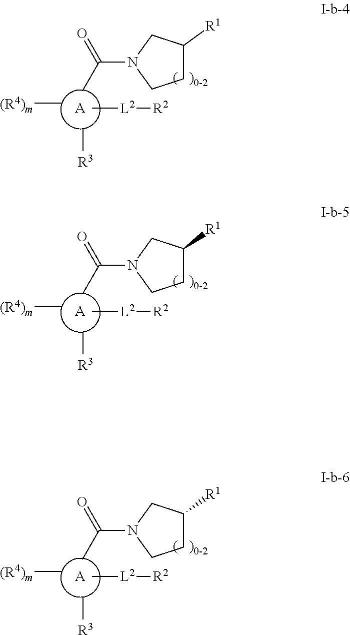

or an N-oxide, or a pharmaceutically acceptable salt thereof, wherein each of Ring A, L², R¹, R², R³, R⁴, and m is as defined above and described in embodiments herein, both singly and in combination.

In some embodiments, the present invention provides a compound of formula I, wherein L² is C₄₋₆ alkylene optionally substituted with one —OH or —F, to provide a compound of formula I-c-1, I-c-2, I-c-3, I-c-4, I-c-5, I-c-6, I-c-7, I-c-8, I-c-9, or I-c-10:

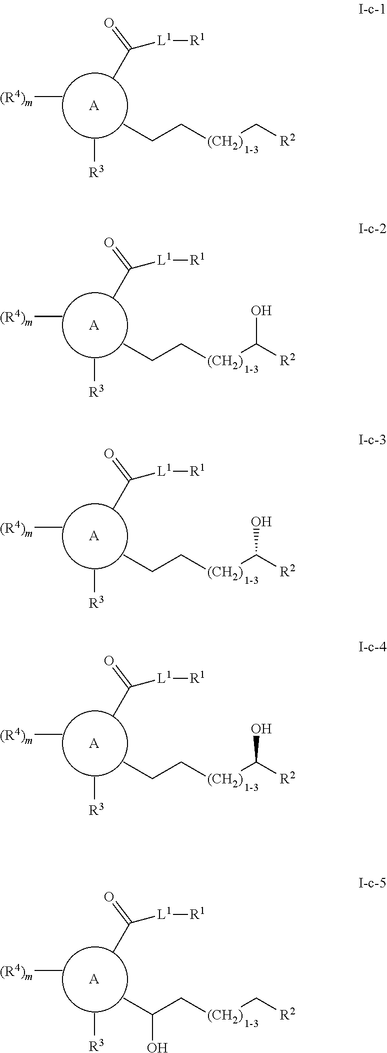

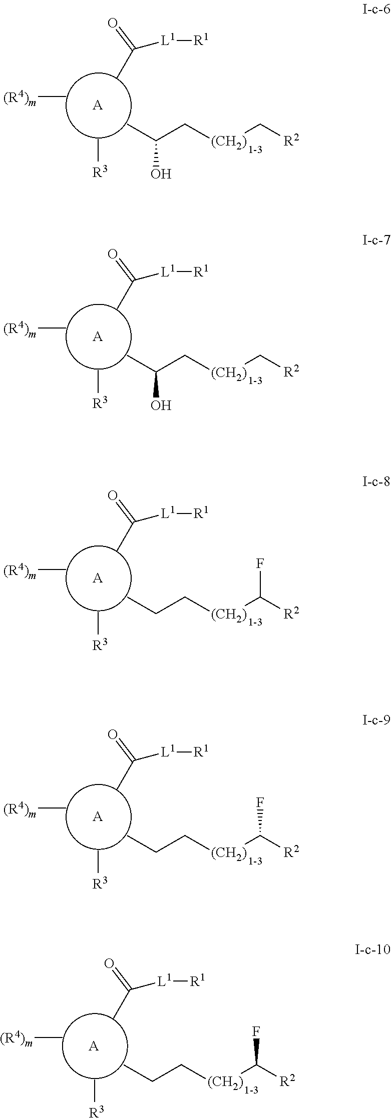

or an N-oxide, or a pharmaceutically acceptable salt thereof, wherein each of Ring A, L¹, R¹, R², R³, R⁴, and m is as defined above and described in embodiments herein, both singly and in combination.

In some embodiments, the present invention provides a compound of formula I wherein L² is

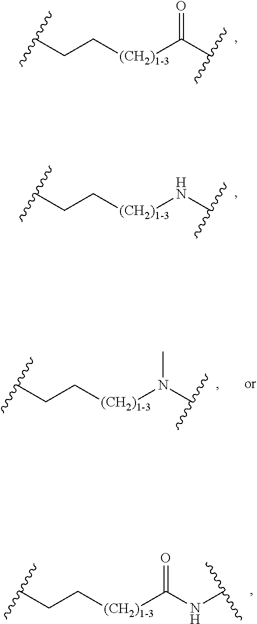

to provide a compound of formula I-c-11, I-c-12, I-c-13, or I-c-14:

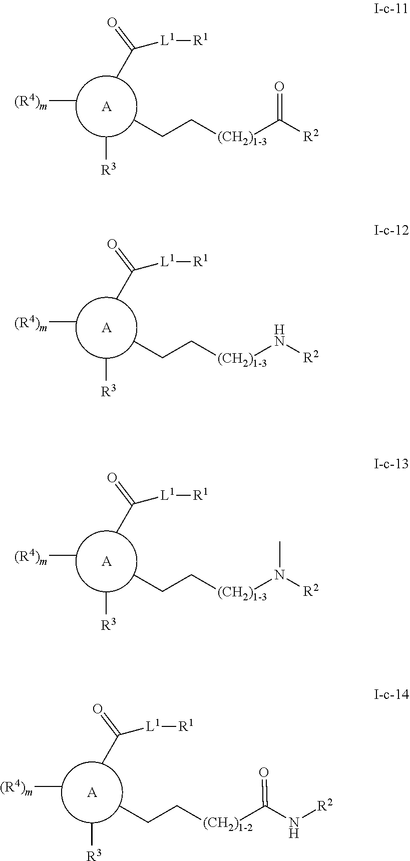

or an N-oxide, or a pharmaceutically acceptable salt thereof, wherein each of Ring A, L¹, R¹, R², R³, R⁴, and m is as defined above and described in embodiments herein, both singly and in combination.

In some embodiments, the present invention provides a compound of formula I, wherein L¹ is hexylene, wherein one methylene units of the chain is independently replaced with —O—, to provide a compound of formula I-c-15, I-c-16, I-c-17, I-c-18, or I-c-19:

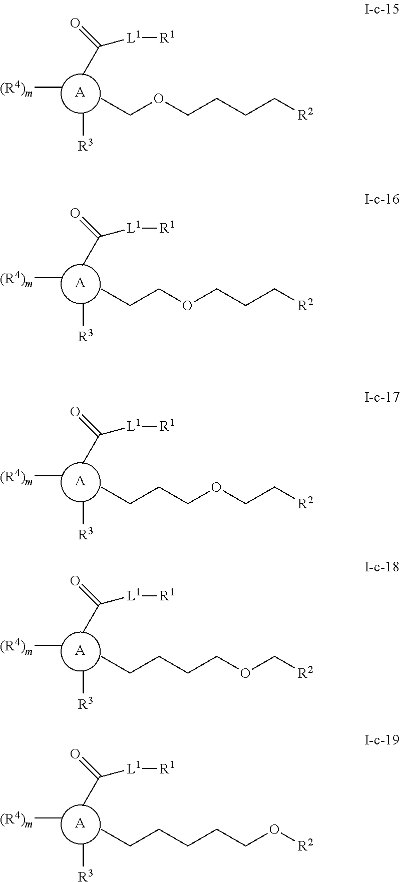

or an N-oxide, or a pharmaceutically acceptable salt thereof, wherein each of Ring A, L¹, R¹, R², R³, R⁴, and m is as defined above and described in embodiments herein, both singly and in combination.

In some embodiments, the present invention provides a compound of formula I, wherein L¹ is

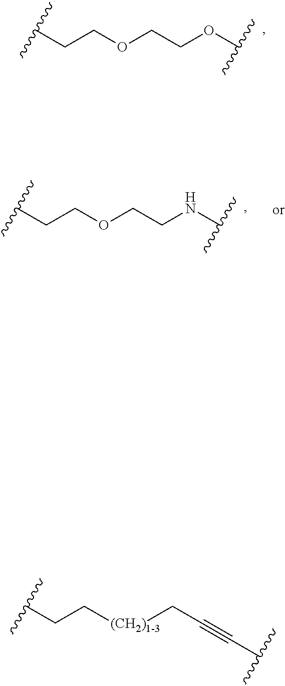 [SMILES: *CCOCCO* ; *CCOCCN* ; *C#CCCCC*]

to provide a compound of formula I-c-20, I-c-21, or I-c-22:

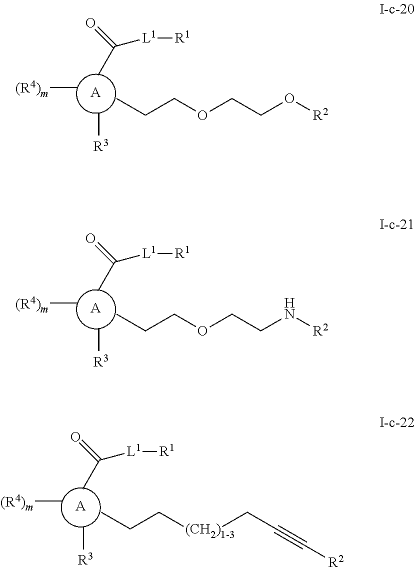

or an N-oxide, or a pharmaceutically acceptable salt thereof, wherein each of Ring A, L¹, R¹, R², R³, R⁴, and m is as defined above and described in embodiments herein, both singly and in combination.

In some embodiments, the present invention provides a compound of formula I, wherein R¹ is carboxyl, tetrazolyl, or

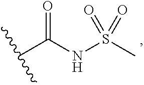 [SMILES: *C(=O)NS(C)(=O)=O]

to provide a compound of formula I-d-1, I-d-2, or I-d-3:

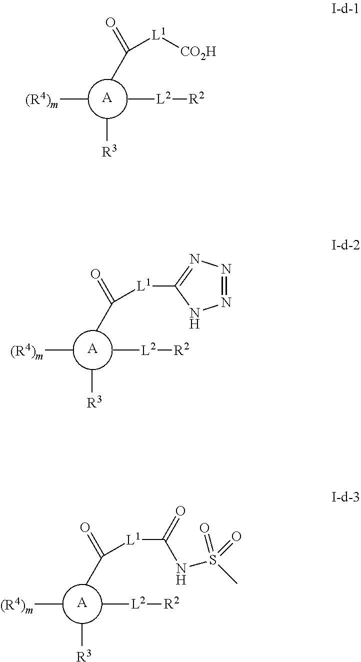

or an -oxide, or a pharmaceutically acceptable salt thereof, wherein each of Ring A, L¹, L², R², R³, R⁴, and m is as defined above and described in embodiments herein, both singly and in combination.

In some embodiments, the present invention provides a compound of formula I, wherein R² is phenyl, pydinyl, pyrimidinyl, or pyrazinyl; each of which is substituted with p instances of R⁶, to provide a compound of formula I-e-1, I-e-2, I-e-3, or I-e-4:

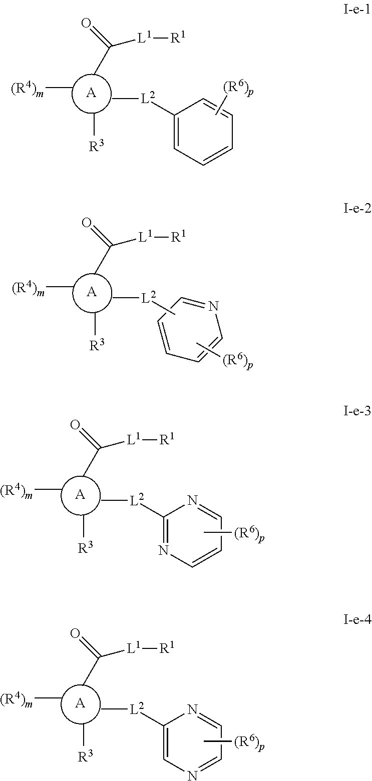

or an N-oxide, or a pharmaceutically acceptable salt thereof, wherein each of Ring A, L¹, L², R¹, R³, R⁴, R⁶, m, and p is as defined above and described in embodiments herein, both singly and in combination.

In some embodiments, the present invention provides a compound of formula I, wherein R² is methyl, ethyl, iso-propyl, tert-butyl, —CF₃, or

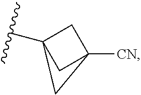 [SMILES: *C12CC(C#N)(C1)C2]

to provide a compound of formula I-e-5, I-e-6, I-e-7, I-e-8, I-e-9, or I-e-10:

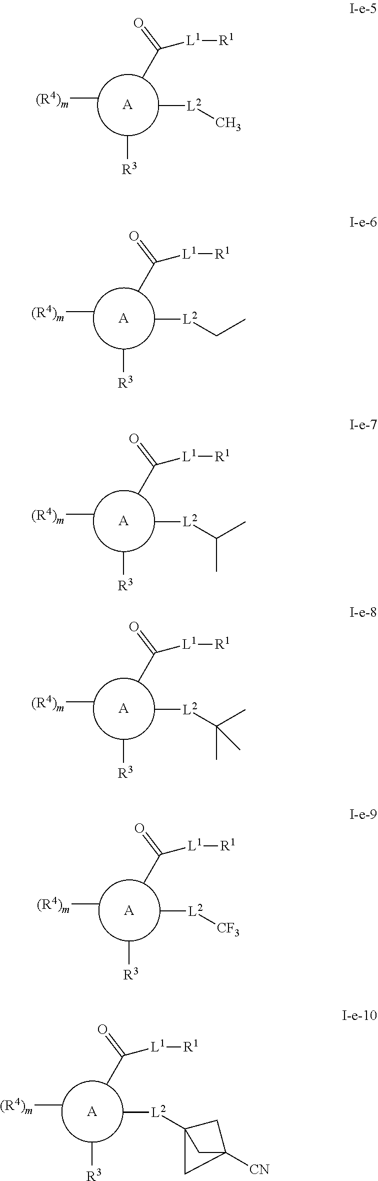

or an N-oxide, or a pharmaceutically acceptable salt thereof, wherein each of Ring A, L¹, L², R¹, R³, R⁴, and m is as defined above and described in embodiments herein, both singly and in combination.

In some embodiments, the present invention provides a compound of formula I, wherein the compound is of formula I-f-1, I-f-2, I-f-3, I-f-4, I-f-5, or I-f-6:

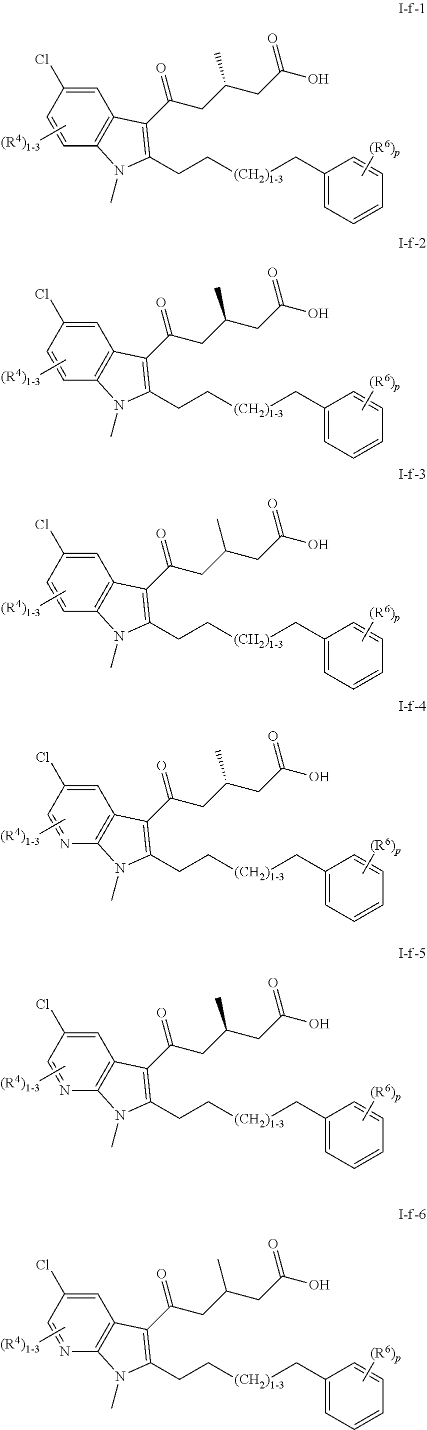

or an N-oxide, or a pharmaceutically acceptable salt thereof, wherein each of R⁴, R⁶, and p is as defined above and described in embodiments herein, both singly and in combination.

In some embodiments, the present invention provides a compound of formula I, wherein the compound is of formula I-g-1, I-g-2, I-g-3, I-g-4, I-g-5, or I-g-6:

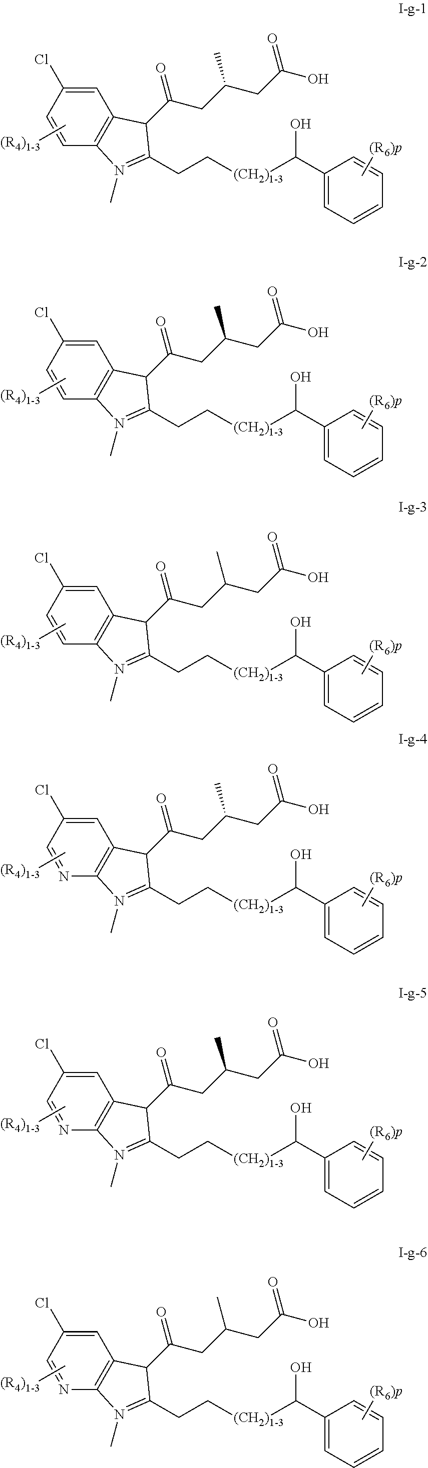

or an N-oxide, or a pharmaceutically acceptable salt thereof, wherein each of R⁴, R⁶, and p is as defined above and described in embodiments herein, both singly and in combination.

In some embodiments, the present invention provides a compound of formula I, wherein the compound is of formula I-h-1, I-h-2, I-h-3, I-h-4, I-h-5, or I-h-6:

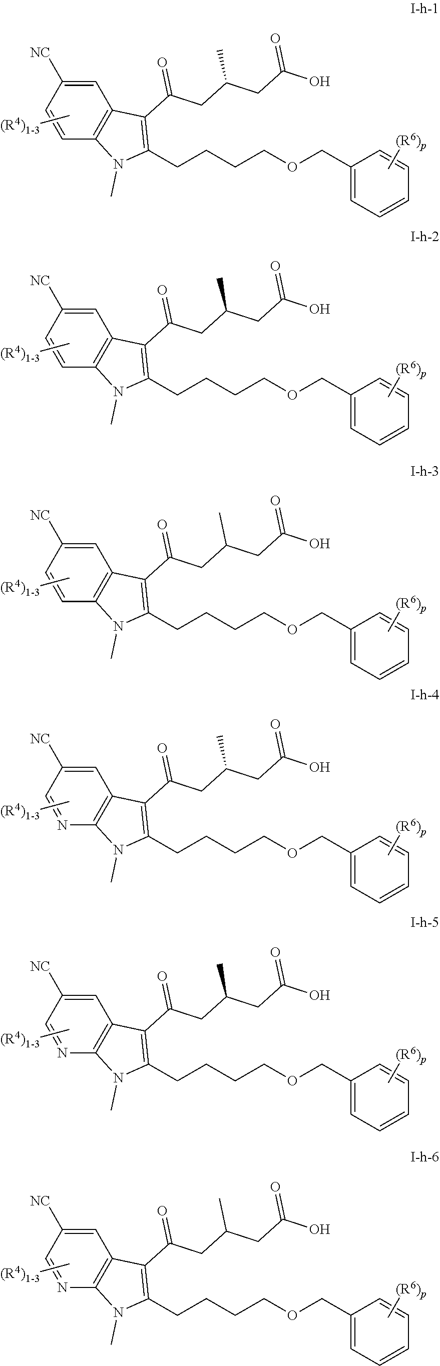

or an N-oxide, or a pharmaceutically acceptable salt thereof, wherein each of R⁴, R⁶, and p is as defined above and described in embodiments herein, both singly and in combination.

In some embodiments, the present invention provides a compound of formula I, wherein the compound is of formula I-i-1, I-i-2, I-i-3, I-i-4, I-i-5, or I-i-6:

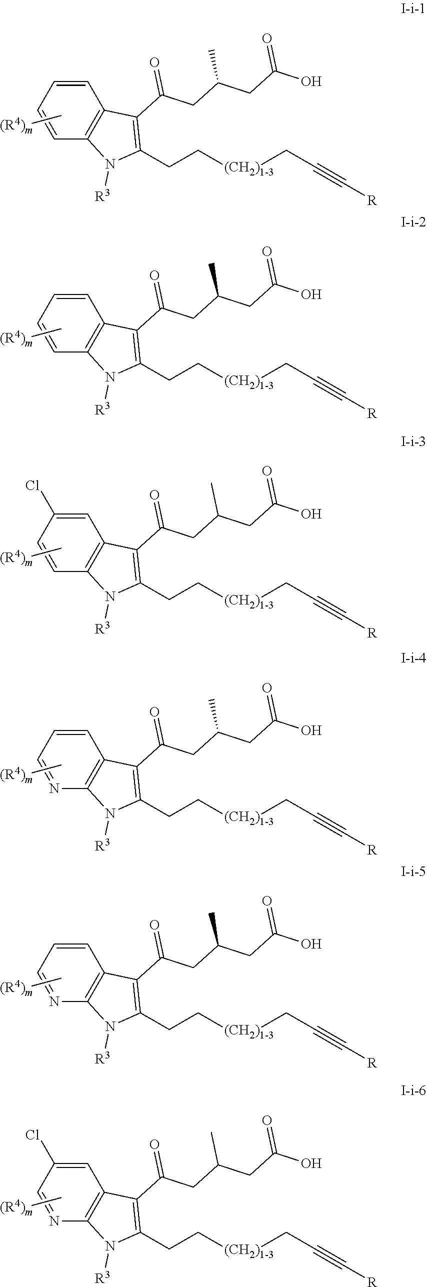

or an N-oxide, or a pharmaceutically acceptable salt thereof, wherein each of R, R³, R⁴, and m is as defined above and described in embodiments herein, both singly and in combination.

In some embodiments, the present invention provides a compound of formula I, wherein the compound is of formula I-j-1, I-j-2, I-j-3, I-j-4, I-j-5, or I-j-6:

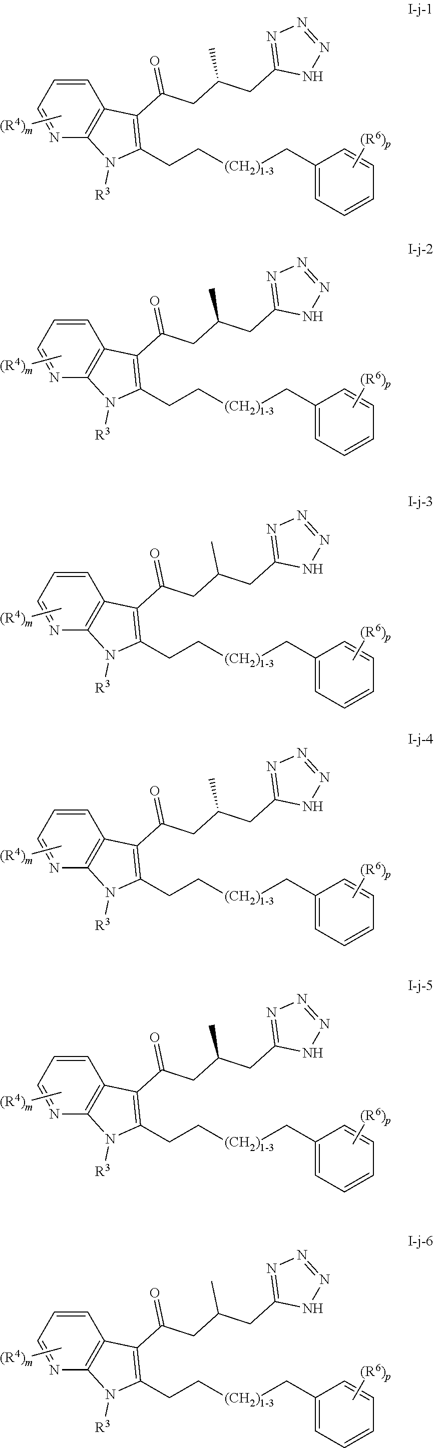

or an N-oxide, or a pharmaceutically acceptable salt thereof, wherein each of R³, R⁴, R⁶, m, and p is as defined above and described in embodiments herein, both singly and in combination.

In some embodiments, the present invention provides a compound of formula I, wherein the compound is of formula I-j-7, I-j-8, I-j-9, I-j-10, I-j-11, or I-j-12:

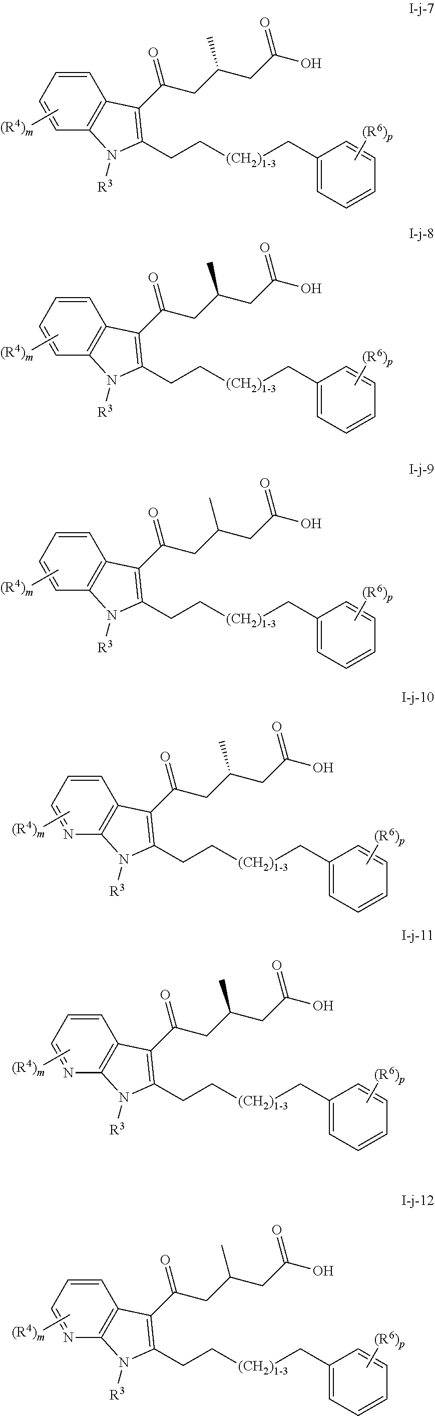

or an N-oxide, or a pharmaceutically acceptable salt thereof, wherein each of R³, R⁴, R⁶, m, and p is as defined above and described in embodiments herein, both singly and in combination.

In some embodiments, the present invention provides a compound of formula I, wherein the compound is of formula I-k-1, I-k-2, I-k-3, I-k-4, I-k-5, or I-k-6:

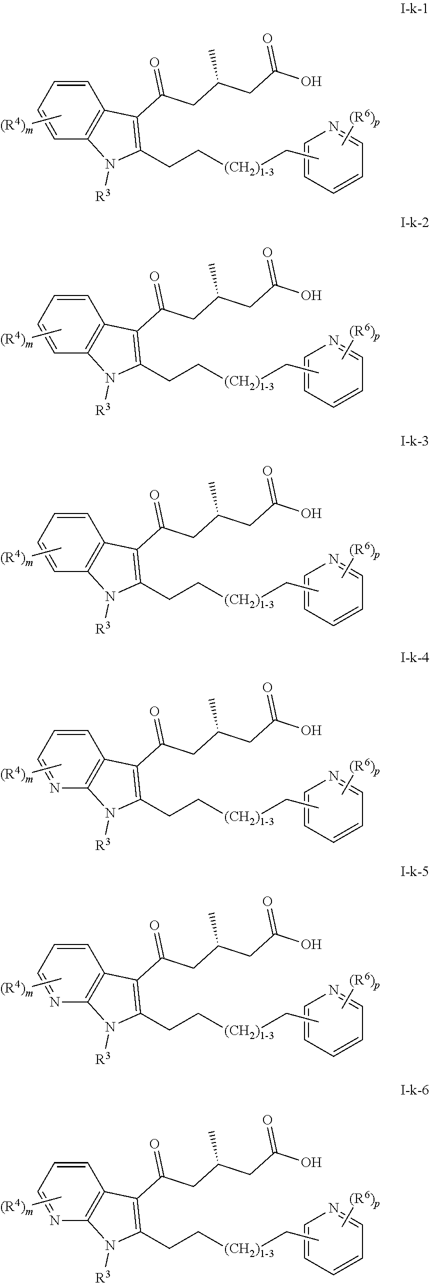

or an N-oxide, or a pharmaceutically acceptable salt thereof, wherein each of R³, R⁴, R⁶, m, and p is as defined above and described in embodiments herein, both singly and in combination.

In some embodiments, the present invention provides a compound of formula I, wherein the compound is of formula I-q-1, I-q-2, I-q-3, I-q-4, I-q-5, or I-q-6:

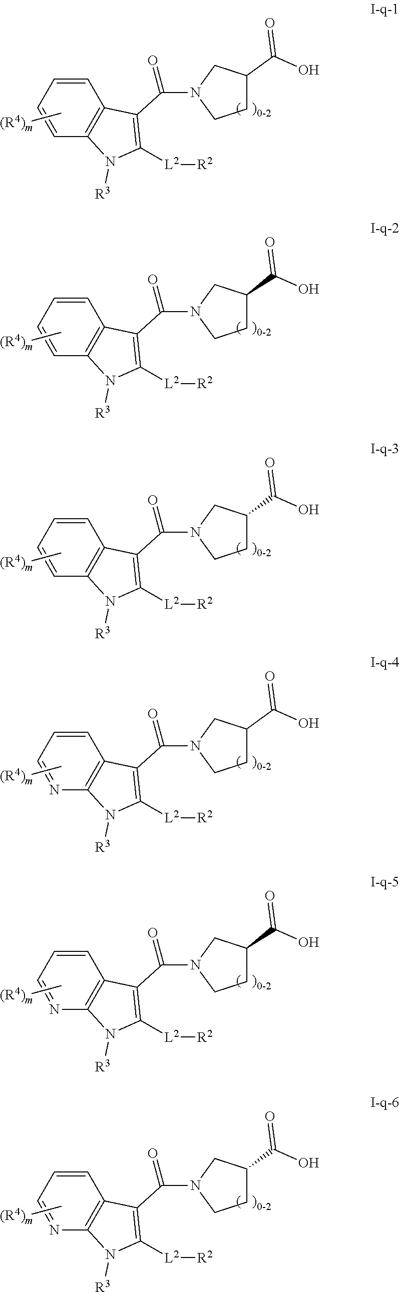

or an N-oxide, or a pharmaceutically acceptable salt thereof, wherein each of L², R², R³, R⁴, and m is as defined above and described in embodiments herein, both singly and in combination.

In some embodiments, the present invention provides a compound of formula I, wherein the compound is of any one of formulae I-q-7, I-q-8, I-q-9, or I-q-10:

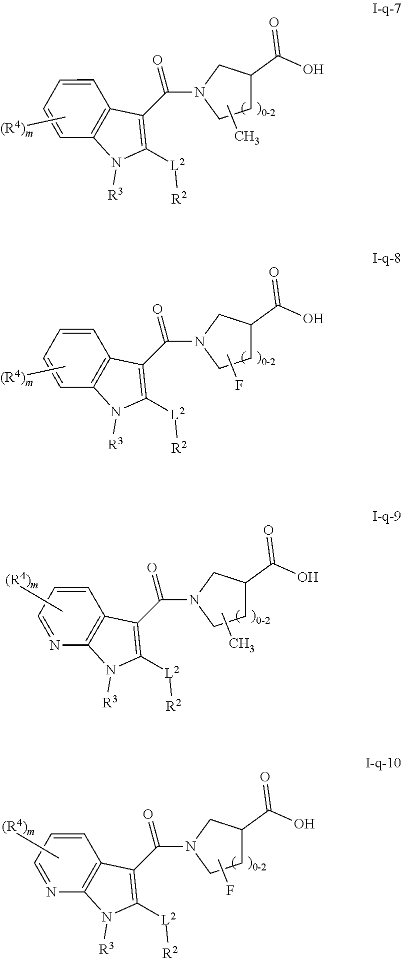

or a pharmaceutically acceptable salt thereof, wherein each of L², R², R³, R⁴, and m is as defined above and described in embodiments herein, both singly and in combination.

In some embodiments, the present invention provides a compound of formula I, wherein the compound is of formulae I-q-11:

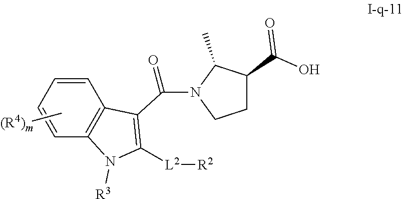

or a pharmaceutically acceptable salt thereof, wherein each of L², R², R³, R⁴, and m is as defined above and described in embodiments herein, both singly and in combination.

In some embodiments, the present invention provides a compound of formula I, wherein the compound is of formula I-r-1, I-r-2, I-r-3, I-r-4, I-r-5, or I-r-6:

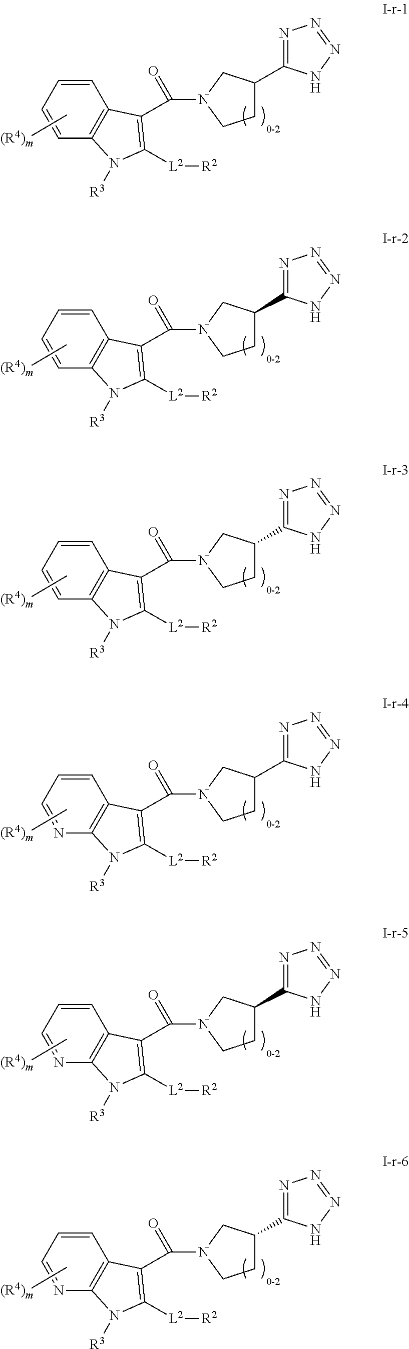

or an N-oxide, or a pharmaceutically acceptable salt thereof, wherein each of L², R², R³, R⁴, and m is as defined above and described in embodiments herein, both singly and in combination.

In some embodiments, the present invention provides a compound of formula I, wherein the compound is of any one of formulae I-r-7, I-r-8, I-r-9:

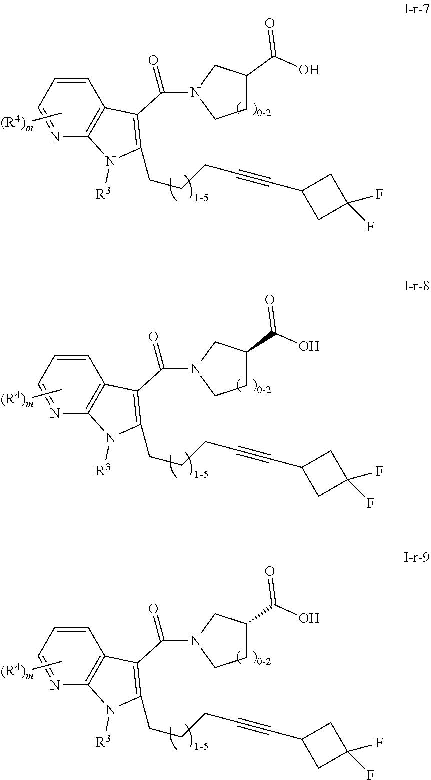

or a pharmaceutically acceptable salt thereof, wherein the variables are as defined above and described in embodiments herein.

In some embodiments, the present invention provides a compound of formula I, wherein the compound is of formulae I-r-10:

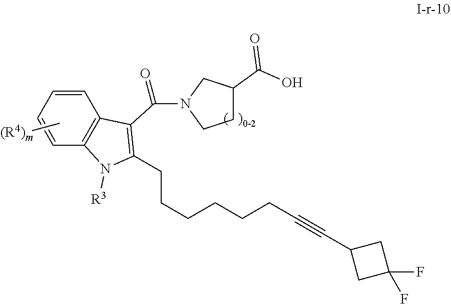

or a pharmaceutically acceptable salt thereof, wherein the variables are as defined above and described in embodiments herein.

Another aspect the invention provides a compound of formula I-1:

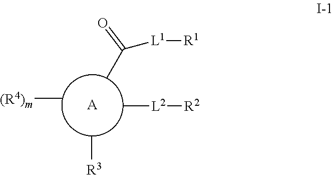

or an N-oxide, or a pharmaceutically acceptable salt thereof, wherein:
Ring A is an 8-10 membered aromatic or partially aromatic bicyclic ring having 1-4 heteroatoms independently selected from nitrogen, oxygen, and sulfur; a 5-6 membered monocyclic heteroaryl ring having 1-4 heteroatoms independently selected from nitrogen, oxygen, and sulfur; or phenyl;
L¹ is one of the following:
   (a) a C₁₋₅ bivalent straight or branched, saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —C(F)₂—, —N(R)—, —S—, —S(O)—, or —S(O)₂—; or
   (b) a covalent bond;
each R is independently hydrogen, or an optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur;
each -Cy- is independently an optionally substituted bivalent ring selected from a 4-7 membered saturated or partially unsaturated heterocyclylenyl having 1-2 heteroatoms independently selected from nitrogen, oxygen, and sulfur, phenylenyl, a 3-7 membered saturated or partially unsaturated carbocyclylenyl, or a 5-6 membered heteroarylenyl having 1-4 heteroatoms independently selected from nitrogen, oxygen, and sulfur;
L² is one of the following:
   (a) a C₁₋₇ bivalent straight or branched saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —CH(F)—, —C(F)₂—, —N(R)—, S—, —S(O)—, or —S(O)₂—; or
   (b) a covalent bond;
R¹ is selected from (i) —C(O)OR, —C(O)N(R)S(O)₂R, and —C(O)N(R)OR; and (ii) a 5-6 membered monocyclic heteroaryl ring having 1-4 heteroatoms independently selected from nitrogen, oxygen, and sulfur; which is substituted with n instances of R⁵;
R² is one of the following:
   (a) phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered aromatic or partially aromatic bicyclic ring having 1-4 heteroatoms independently selected from nitrogen, oxygen, and sulfur; each of which is substituted with p instances of R⁶; or
   (b) C₁₋₇ aliphatic, —C≡CR, —C(O)OR, or —C(O)R; each of which is substituted with p instances of R⁶; or
   (c) hydrogen;
R³ is one of the following:
   (a) a C₁₋₆ aliphatic group optionally substituted with one or more —OH or —N(R)₂;
   (b) —CD₃;
   (c) hydrogen; or
   (d) absent;
each instance of R⁴ is independently hydrogen, deuterium, R^{z}, —C≡CR, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂; or
   two R⁴ groups are optionally taken together to form ═O; or
   two R⁴ groups are optionally taken together with their intervening atoms to form an optionally substituted 5-8 membered saturated, partially unsaturated, or aryl fused ring having 0-3 heteroatoms independently selected from nitrogen, oxygen and sulfur;
each instance of R⁵, and R⁶ is independently hydrogen, deuterium, R^{z}, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂; or
   two R⁵ groups are optionally taken together to form ═O;
   two R⁶ groups are optionally taken together to form ═O;
   two R⁵ groups are optionally taken together with their intervening atoms to form an optionally substituted 5-8 membered saturated, partially unsaturated, or aryl fused ring having 0-3 heteroatoms independently selected from nitrogen, oxygen and sulfur; or
   two R⁶ groups are optionally taken together with their intervening atoms to form an optionally substituted 5-8 membered saturated, partially unsaturated, or aryl fused ring having 0-3 heteroatoms independently selected from nitrogen, oxygen and sulfur;
each instance of R^{z} is independently selected from an optionally substituted group selected from C₁₋₆ aliphatic; phenyl; a 4-7 membered saturated or partially unsaturated heterocyclic ring having 1-2 heteroatoms independently selected from nitrogen, oxygen, and sulfur; and a 5-6 membered heteroaryl ring having 1-4 heteroatoms independently selected from nitrogen, oxygen, and sulfur;
m is 0, 1, 2, 3, or 4;
n is 0, 1, 2, 3, or 4; and
p is 0, 1, 2, 3, or 4;
provided that if L¹-R¹ is

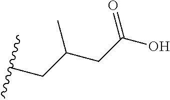 [SMILES: *CC(C)CC(=O)O]

and ring A with its R³ and R⁴ substituents is

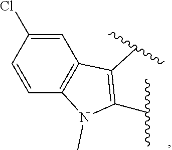 [SMILES: *c1c(*)n(C)c2ccc(Cl)cc12]

then L²-R² is not n-hexyl,

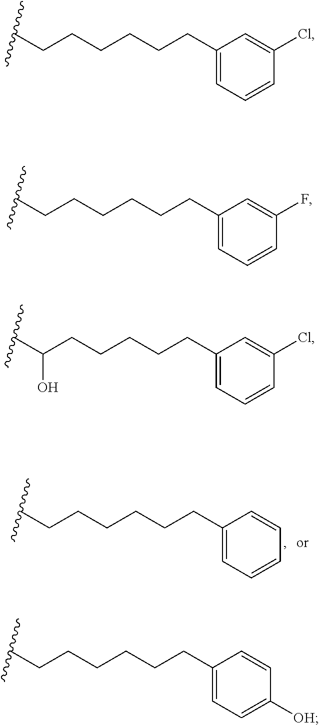 [SMILES: *CCCCCCc1cccc(Cl)c1 ; *CCCCCCc1cccc(F)c1 ; *C(O)CCCCCc1cccc(Cl)c1 ; *CCCCCCc1ccccc1 ; *CCCCCCc1ccc(O)cc1]

and
provided that if L¹-R¹ is

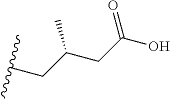 [SMILES: *C[C@H](C)CC(=O)O]

and ring A with its R³ and R⁴ substituents is

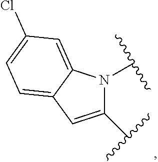 [SMILES: *c1cc2ccc(Cl)cc2n1*]

then L²-R² is not n-hexyl.

In certain embodiments, the definition of one or more of variables R¹, R², R³, R⁴, L¹, L², Ring A, or m is one of the embodiments set forth above in connection with Formula I.

In certain embodiments, the compound has the benefit of low binding affinity to plasma proteins in subjects. Low binding affinity to plasma proteins can result in a higher concentration of free compound in the subject, thereby leading to a superior therapeutic effect. Binding of a compound to plasma proteins can be analyzed according to procedures described in the literature for evaluating compound binding to plasma proteins.

Exemplary compounds of the invention are set forth in Table 1, below.

TABLE 1
Exemplary Compounds

| Compound No. | Structure |
|---|---|
| I-13 | 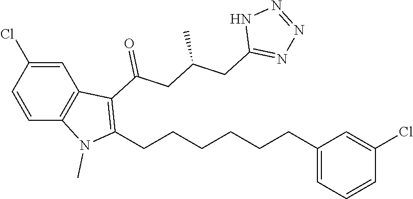 [SMILES: C[C@@H](CC(=O)c1c(CCCCCCc2cccc(Cl)c2)n(C)c2ccc(Cl)cc12)Cc1nnn[nH]1] |
| I-14 | 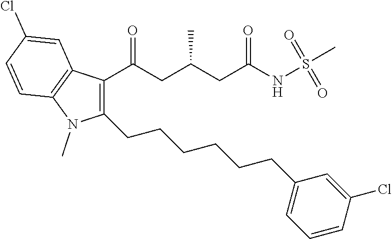 [SMILES: C[C@H](CC(=O)NS(C)(=O)=O)CC(=O)c1c(CCCCCCc2cccc(Cl)c2)n(C)c2ccc(Cl)cc12] |
| I-15 | 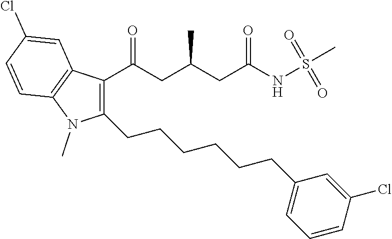 [SMILES: C[C@@H](CC(=O)NS(C)(=O)=O)CC(=O)c1c(CCCCCCc2cccc(Cl)c2)n(C)c2ccc(Cl)cc12] |
| I-16 | 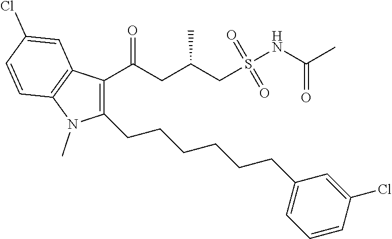 [SMILES: CC(=O)NS(=O)(=O)C[C@@H](C)CC(=O)c1c(CCCCCCc2cccc(Cl)c2)n(C)c2ccc(Cl)cc12] |
| I-17 | 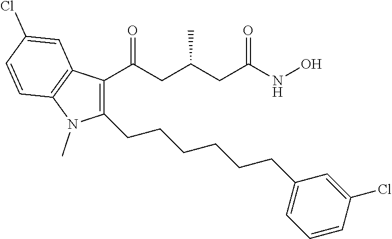 [SMILES: C[C@H](CC(=O)NO)CC(=O)c1c(CCCCCCc2cccc(Cl)c2)n(C)c2ccc(Cl)cc12] |
| I-18 | 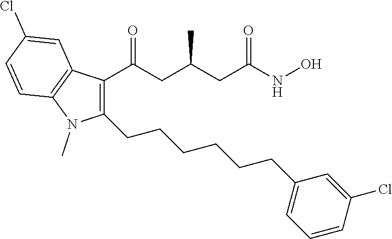 [SMILES: C[C@@H](CC(=O)NO)CC(=O)c1c(CCCCCCc2cccc(Cl)c2)n(C)c2ccc(Cl)cc12] |
| I-19 | 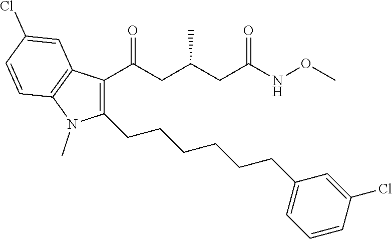 [SMILES: CONC(=O)C[C@@H](C)CC(=O)c1c(CCCCCCc2cccc(Cl)c2)n(C)c2ccc(Cl)cc12] |
| I-20 | 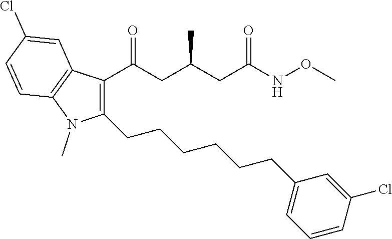 [SMILES: CONC(=O)C[C@H](C)CC(=O)c1c(CCCCCCc2cccc(Cl)c2)n(C)c2ccc(Cl)cc12] |
| I-21 | 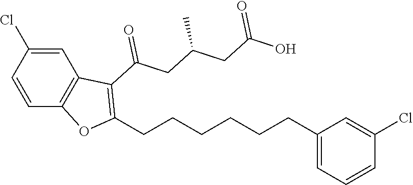 [SMILES: C[C@H](CC(=O)O)CC(=O)c1c(CCCCCCc2cccc(Cl)c2)oc2ccc(Cl)cc12] |
| I-22 | 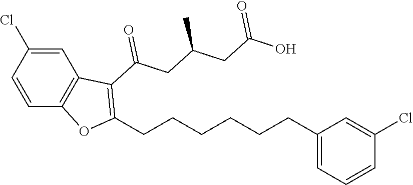 [SMILES: C[C@@H](CC(=O)O)CC(=O)c1c(CCCCCCc2cccc(Cl)c2)oc2ccc(Cl)cc12] |
| I-23 | 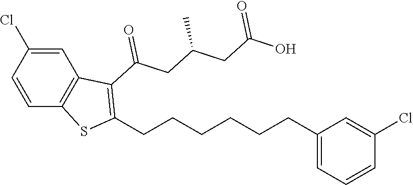 [SMILES: C[C@H](CC(=O)O)CC(=O)c1c(CCCCCCc2cccc(Cl)c2)sc2ccc(Cl)cc12] |
| I-24 | 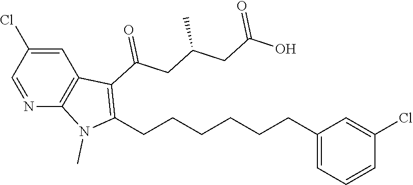 [SMILES: C[C@H](CC(=O)O)CC(=O)c1c(CCCCCCc2cccc(Cl)c2)n(C)c2ncc(Cl)cc12] |
| I-25 | 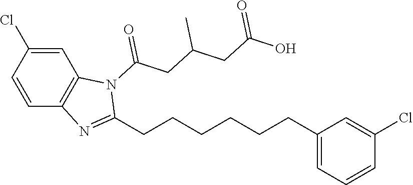 [SMILES: CC(CC(=O)O)CC(=O)n1c(CCCCCCc2cccc(Cl)c2)nc2ccc(Cl)cc21] |
| I-26 | 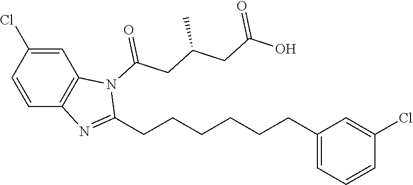 [SMILES: C[C@H](CC(=O)O)CC(=O)n1c(CCCCCCc2cccc(Cl)c2)nc2ccc(Cl)cc21] |
| I-27 | 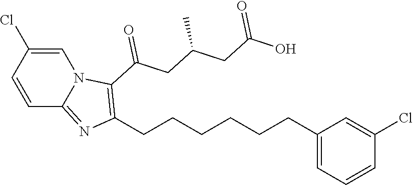 [SMILES: C[C@H](CC(=O)O)CC(=O)c1c(CCCCCCc2cccc(Cl)c2)nc2ccc(Cl)cn12] |
| I-28 | 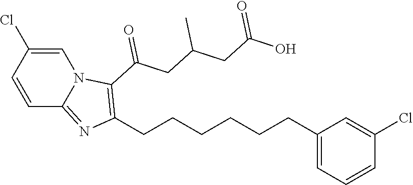 [SMILES: CC(CC(=O)O)CC(=O)c1c(CCCCCCc2cccc(Cl)c2)nc2ccc(Cl)cn12] |
| I-29 | 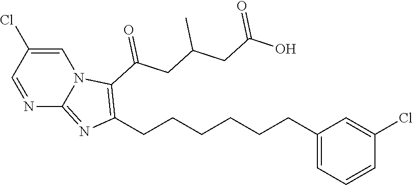 [SMILES: CC(CC(=O)O)CC(=O)c1c(CCCCCCc2cccc(Cl)c2)nc2ncc(Cl)cn12] |
| I-30 | 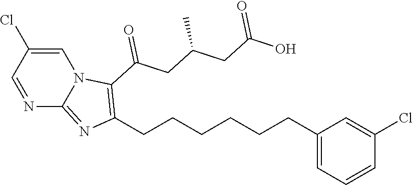 [SMILES: C[C@H](CC(=O)O)CC(=O)c1c(CCCCCCc2cccc(Cl)c2)nc2ncc(Cl)cn12] |
| I-31 | 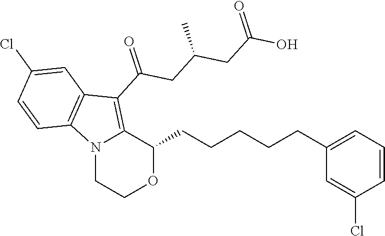 [SMILES: C[C@H](CC(=O)O)CC(=O)c1c2n(c3ccc(Cl)cc13)CCO[C@H]2CCCCCc1cccc(Cl)c1] |
| I-32 | 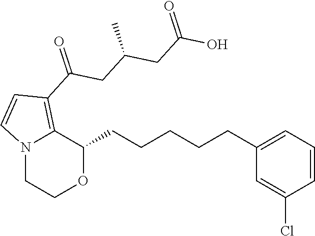 [SMILES: C[C@H](CC(=O)O)CC(=O)c1ccn2c1[C@H](CCCCCc1cccc(Cl)c1)OCC2] |
| I-33 | 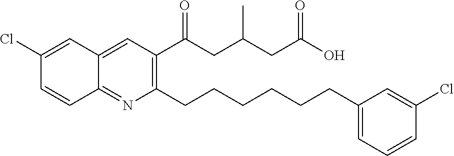 [SMILES: CC(CC(=O)O)CC(=O)c1cc2cc(Cl)ccc2nc1CCCCCCc1cccc(Cl)c1] |
| I-34 | 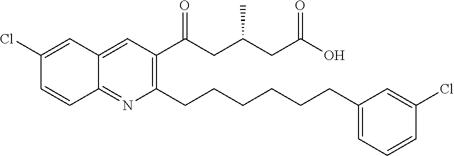 [SMILES: C[C@H](CC(=O)O)CC(=O)c1cc2cc(Cl)ccc2nc1CCCCCCc1cccc(Cl)c1] |
| I-35 | 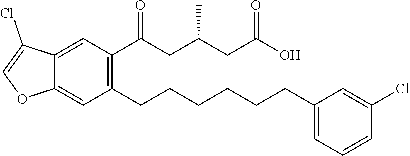 [SMILES: C[C@H](CC(=O)O)CC(=O)c1cc2c(Cl)coc2cc1CCCCCCc1cccc(Cl)c1] |
| I-36 | 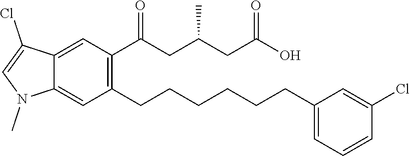 [SMILES: C[C@H](CC(=O)O)CC(=O)c1cc2c(Cl)cn(C)c2cc1CCCCCCc1cccc(Cl)c1] |
| I-37 | 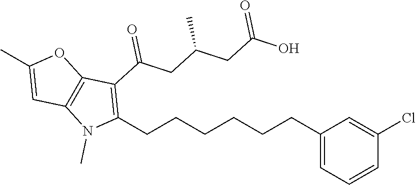 [SMILES: Cc1cc2c(o1)c(C(=O)C[C@H](C)CC(=O)O)c(CCCCCCc1cccc(Cl)c1)n2C] |
| I-38 | 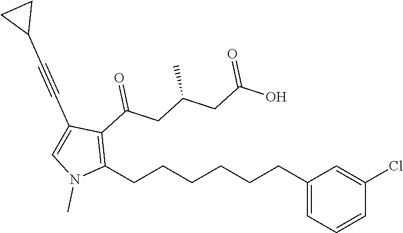 [SMILES: C[C@H](CC(=O)O)CC(=O)c1c(C#CC2CC2)cn(C)c1CCCCCCc1cccc(Cl)c1] |
| I-39 | 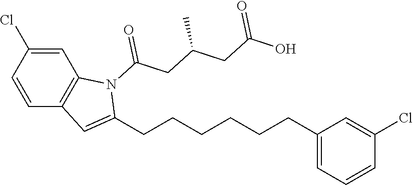 [SMILES: C[C@H](CC(=O)O)CC(=O)n1c(CCCCCCc2cccc(Cl)c2)cc2ccc(Cl)cc21] |
| I-40 | 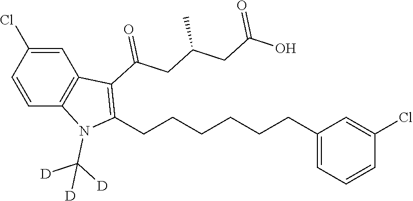 [SMILES: [2H]C([2H])([2H])n1c(CCCCCCc2cccc(Cl)c2)c(C(=O)C[C@H](C)CC(=O)O)c2cc(Cl)ccc21] |
| I-41 | 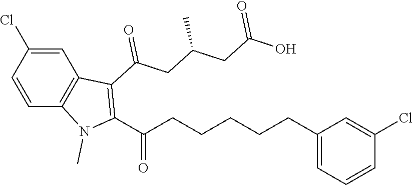 [SMILES: C[C@H](CC(=O)O)CC(=O)c1c(C(=O)CCCCCc2cccc(Cl)c2)n(C)c2ccc(Cl)cc12] |
| I-42 | 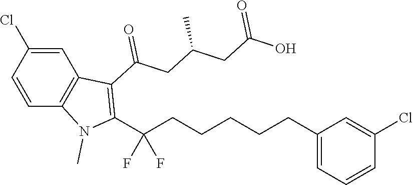 [SMILES: C[C@H](CC(=O)O)CC(=O)c1c(C(F)(F)CCCCCc2cccc(Cl)c2)n(C)c2ccc(Cl)cc12] |
| I-43 | 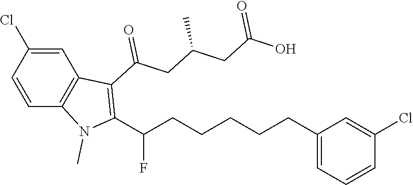 [SMILES: C[C@H](CC(=O)O)CC(=O)c1c(C(F)CCCCCc2cccc(Cl)c2)n(C)c2ccc(Cl)cc12] |
| I-44 | 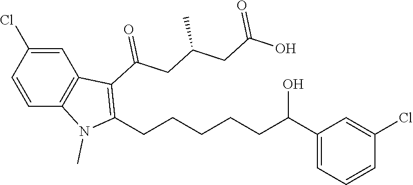 [SMILES: C[C@H](CC(=O)O)CC(=O)c1c(CCCCCC(O)c2cccc(Cl)c2)n(C)c2ccc(Cl)cc12] |
| I-45 | 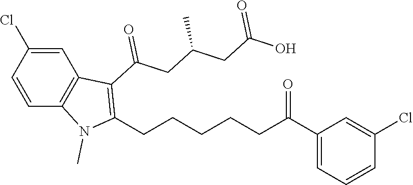 [SMILES: C[C@H](CC(=O)O)CC(=O)c1c(CCCCCC(=O)c2cccc(Cl)c2)n(C)c2ccc(Cl)cc12] |
| I-46 | 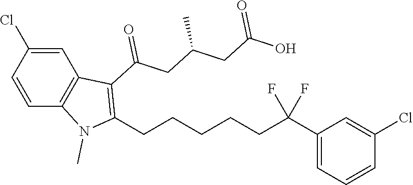 [SMILES: C[C@H](CC(=O)O)CC(=O)c1c(CCCCCC(F)(F)c2cccc(Cl)c2)n(C)c2ccc(Cl)cc12] |
| I-47 | 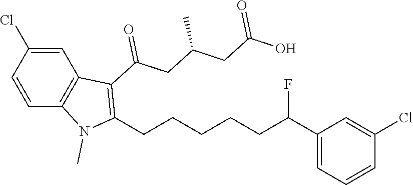 [SMILES: C[C@H](CC(=O)O)CC(=O)c1c(CCCCCC(F)c2cccc(Cl)c2)n(C)c2ccc(Cl)cc12] |
| I-48 | 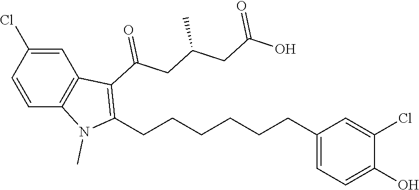 [SMILES: C[C@H](CC(=O)O)CC(=O)c1c(CCCCCCc2ccc(O)c(Cl)c2)n(C)c2ccc(Cl)cc12] |
| I-49 | 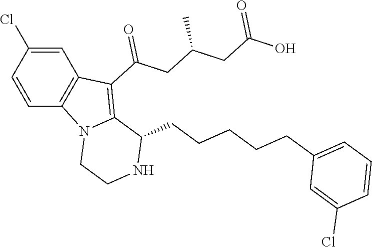 [SMILES: C[C@H](CC(=O)O)CC(=O)c1c2n(c3ccc(Cl)cc13)CCN[C@H]2CCCCCc1cccc(Cl)c1] |
| I-50 | 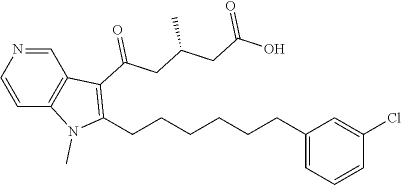 [SMILES: C[C@H](CC(=O)O)CC(=O)c1c(CCCCCCc2cccc(Cl)c2)n(C)c2ccncc12] |
| I-51 | 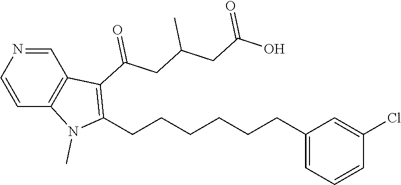 [SMILES: CC(CC(=O)O)CC(=O)c1c(CCCCCCc2cccc(Cl)c2)n(C)c2ccncc12] |
| I-52 | 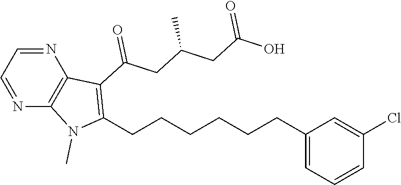 [SMILES: C[C@H](CC(=O)O)CC(=O)c1c(CCCCCCc2cccc(Cl)c2)n(C)c2nccnc12] |
| I-53 | 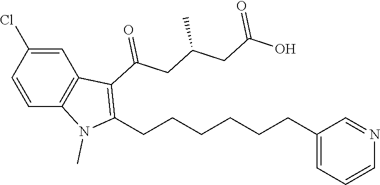 [SMILES: C[C@H](CC(=O)O)CC(=O)c1c(CCCCCCc2cccnc2)n(C)c2ccc(Cl)cc12] |
| I-54 | 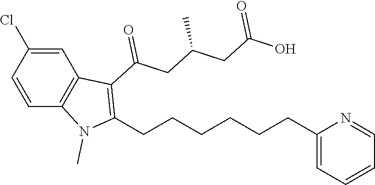 [SMILES: C[C@H](CC(=O)O)CC(=O)c1c(CCCCCCc2ccccn2)n(C)c2ccc(Cl)cc12] |
| I-55 | 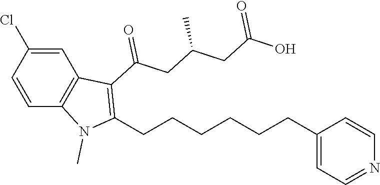 [SMILES: C[C@H](CC(=O)O)CC(=O)c1c(CCCCCCc2ccncc2)n(C)c2ccc(Cl)cc12] |
| I-56 | 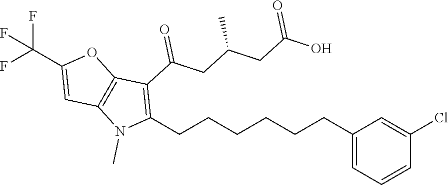 [SMILES: C[C@H](CC(=O)O)CC(=O)c1c(CCCCCCc2cccc(Cl)c2)n(C)c2cc(C(F)(F)F)oc12] |
| I-57 | 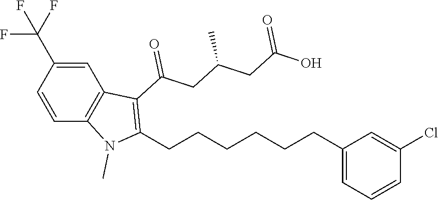 [SMILES: C[C@H](CC(=O)O)CC(=O)c1c(CCCCCCc2cccc(Cl)c2)n(C)c2ccc(C(F)(F)F)cc12] |
| I-58 | 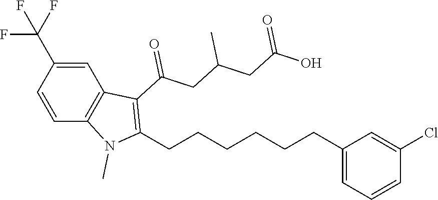 [SMILES: CC(CC(=O)O)CC(=O)c1c(CCCCCCc2cccc(Cl)c2)n(C)c2ccc(C(F)(F)F)cc12] |
| I-59 | 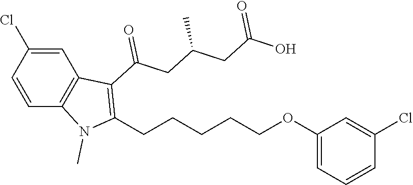 [SMILES: C[C@H](CC(=O)O)CC(=O)c1c(CCCCCOc2cccc(Cl)c2)n(C)c2ccc(Cl)cc12] |
| I-60 | 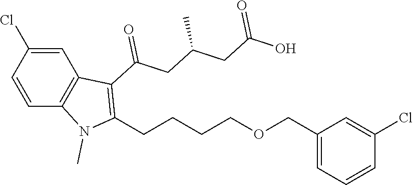 [SMILES: C[C@H](CC(=O)O)CC(=O)c1c(CCCCOCc2cccc(Cl)c2)n(C)c2ccc(Cl)cc12] |
| I-61 | 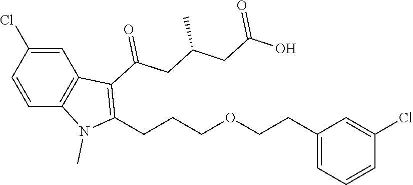 [SMILES: C[C@H](CC(=O)O)CC(=O)c1c(CCCOCCc2cccc(Cl)c2)n(C)c2ccc(Cl)cc12] |
| I-62 | 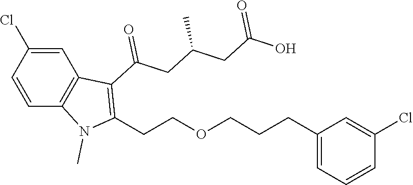 [SMILES: C[C@H](CC(=O)O)CC(=O)c1c(CCOCCCc2cccc(Cl)c2)n(C)c2ccc(Cl)cc12] |
| I-63 | 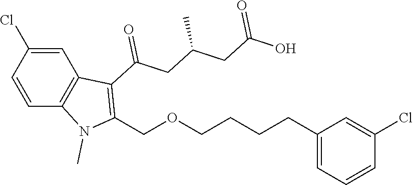 [SMILES: C[C@H](CC(=O)O)CC(=O)c1c(COCCCCc2cccc(Cl)c2)n(C)c2ccc(Cl)cc12] |
| I-64 | 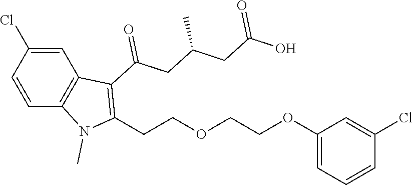 [SMILES: C[C@H](CC(=O)O)CC(=O)c1c(CCOCCOc2cccc(Cl)c2)n(C)c2ccc(Cl)cc12] |
| I-65 | 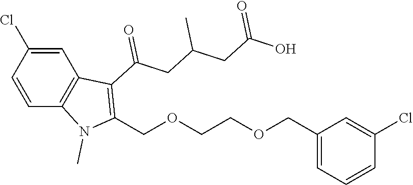 [SMILES: CC(CC(=O)O)CC(=O)c1c(COCCOCc2cccc(Cl)c2)n(C)c2ccc(Cl)cc12] |
| I-66 | 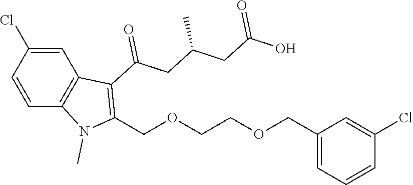 [SMILES: C[C@H](CC(=O)O)CC(=O)c1c(COCCOCc2cccc(Cl)c2)n(C)c2ccc(Cl)cc12] |
| I-67 | 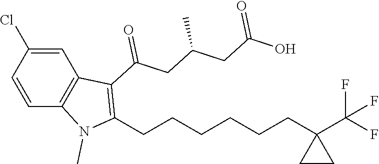 [SMILES: C[C@H](CC(=O)O)CC(=O)c1c(CCCCCCC2(C(F)(F)F)CC2)n(C)c2ccc(Cl)cc12] |
| I-68 | 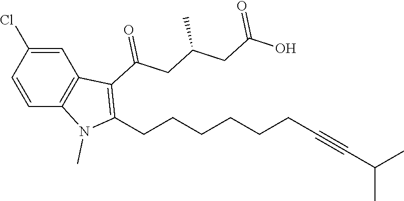 [SMILES: CC(C)C#CCCCCCCc1c(C(=O)C[C@H](C)CC(=O)O)c2cc(Cl)ccc2n1C] |
| I-69 | 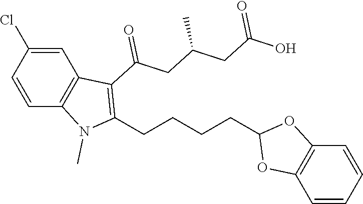 [SMILES: C[C@H](CC(=O)O)CC(=O)c1c(CCCCC2Oc3ccccc3O2)n(C)c2ccc(Cl)cc12] |
| I-70 | 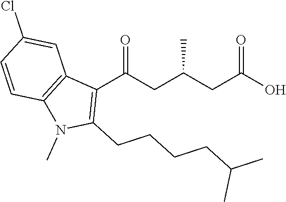 [SMILES: CC(C)CCCCc1c(C(=O)C[C@H](C)CC(=O)O)c2cc(Cl)ccc2n1C] |
| I-71 | 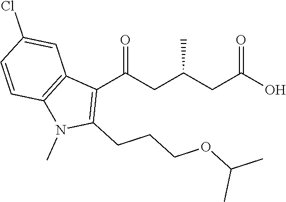 [SMILES: CC(C)OCCCc1c(C(=O)C[C@H](C)CC(=O)O)c2cc(Cl)ccc2n1C] |
| I-72 | 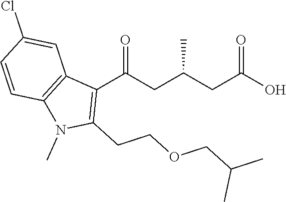 [SMILES: CC(C)COCCc1c(C(=O)C[C@H](C)CC(=O)O)c2cc(Cl)ccc2n1C] |
| I-73 | 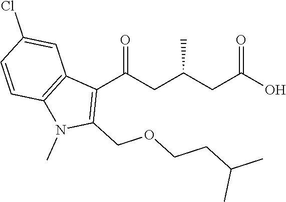 [SMILES: CC(C)CCOCc1c(C(=O)C[C@H](C)CC(=O)O)c2cc(Cl)ccc2n1C] |
| I-74 | 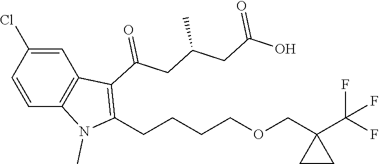 [SMILES: C[C@H](CC(=O)O)CC(=O)c1c(CCCCOCC2(C(F)(F)F)CC2)n(C)c2ccc(Cl)cc12] |
| I-75 | 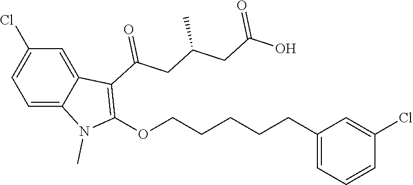 [SMILES: C[C@H](CC(=O)O)CC(=O)c1c(OCCCCCc2cccc(Cl)c2)n(C)c2ccc(Cl)cc12] |
| I-76 | 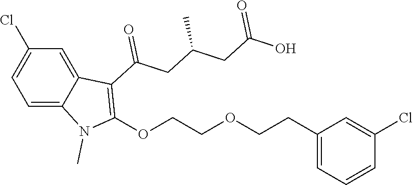 [SMILES: C[C@H](CC(=O)O)CC(=O)c1c(OCCOCCc2cccc(Cl)c2)n(C)c2ccc(Cl)cc12] |
| I-77 | 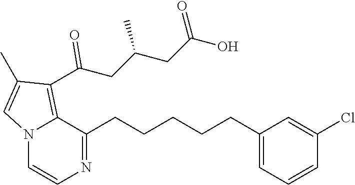 [SMILES: Cc1cn2ccnc(CCCCCc3cccc(Cl)c3)c2c1C(=O)C[C@H](C)CC(=O)O] |
| I-78 | 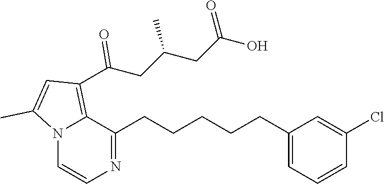 [SMILES: Cc1cc(C(=O)C[C@H](C)CC(=O)O)c2c(CCCCCc3cccc(Cl)c3)nccn12] |
| I-79 | 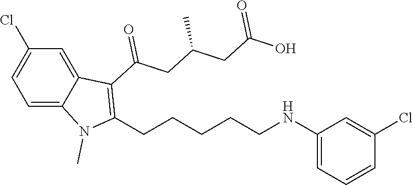 [SMILES: C[C@H](CC(=O)O)CC(=O)c1c(CCCCCNc2cccc(Cl)c2)n(C)c2ccc(Cl)cc12] |
| I-80 | 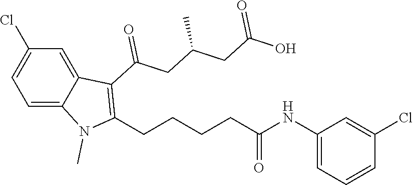 [SMILES: C[C@H](CC(=O)O)CC(=O)c1c(CCCCC(=O)Nc2cccc(Cl)c2)n(C)c2ccc(Cl)cc12] |
| I-81 | 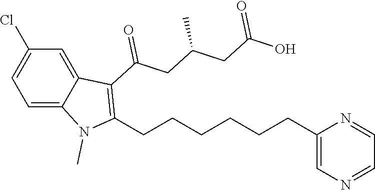 [SMILES: C[C@H](CC(=O)O)CC(=O)c1c(CCCCCCc2cnccn2)n(C)c2ccc(Cl)cc12] |
| I-82 | 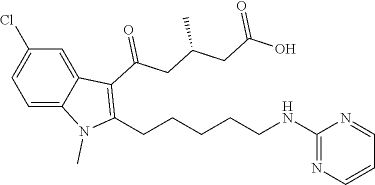 [SMILES: C[C@H](CC(=O)O)CC(=O)c1c(CCCCCNc2ncccn2)n(C)c2ccc(Cl)cc12] |
| I-83 | 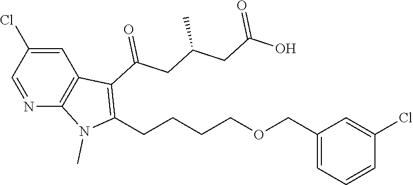 [SMILES: C[C@H](CC(=O)O)CC(=O)c1c(CCCCOCc2cccc(Cl)c2)n(C)c2ncc(Cl)cc12] |
| I-84 | 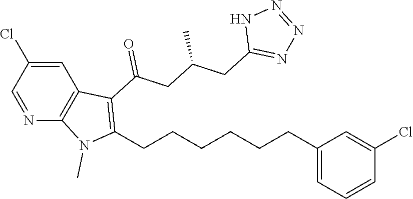 [SMILES: C[C@@H](CC(=O)c1c(CCCCCCc2cccc(Cl)c2)n(C)c2ncc(Cl)cc12)Cc1nnn[nH]1] |
| I-85 | 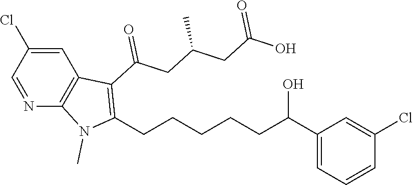 [SMILES: C[C@H](CC(=O)O)CC(=O)c1c(CCCCCC(O)c2cccc(Cl)c2)n(C)c2ncc(Cl)cc12] |
| I-86 | 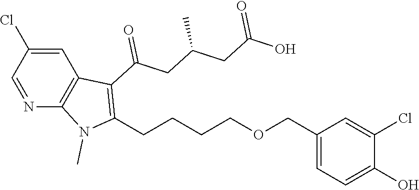 [SMILES: C[C@H](CC(=O)O)CC(=O)c1c(CCCCOCc2ccc(O)c(Cl)c2)n(C)c2ncc(Cl)cc12] |
| I-87 | 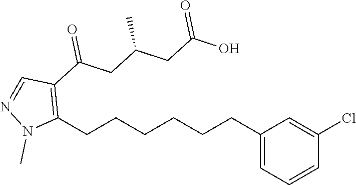 [SMILES: C[C@H](CC(=O)O)CC(=O)c1cnn(C)c1CCCCCCc1cccc(Cl)c1] |
| I-88 | 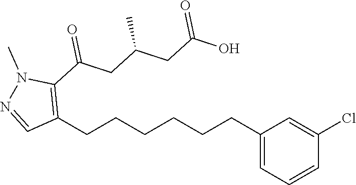 [SMILES: C[C@H](CC(=O)O)CC(=O)c1c(CCCCCCc2cccc(Cl)c2)cnn1C] |
| I-89 | 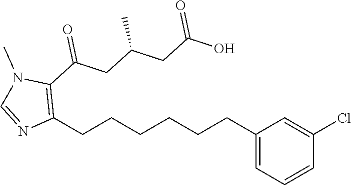 [SMILES: C[C@H](CC(=O)O)CC(=O)c1c(CCCCCCc2cccc(Cl)c2)ncn1C] |
| I-90 | 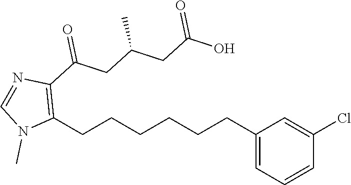 [SMILES: C[C@H](CC(=O)O)CC(=O)c1ncn(C)c1CCCCCCc1cccc(Cl)c1] |
| I-91 | 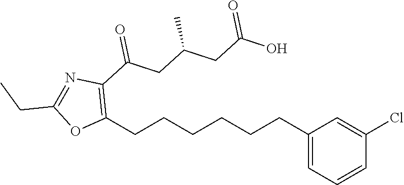 [SMILES: CCc1nc(C(=O)C[C@H](C)CC(=O)O)c(CCCCCCc2cccc(Cl)c2)o1] |
| I-92 | 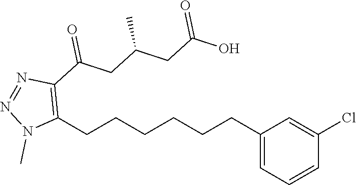 [SMILES: C[C@H](CC(=O)O)CC(=O)c1nnn(C)c1CCCCCCc1cccc(Cl)c1] |
| I-93 | 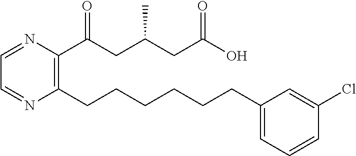 [SMILES: C[C@H](CC(=O)O)CC(=O)c1nccnc1CCCCCCc1cccc(Cl)c1] |
| I-94 | 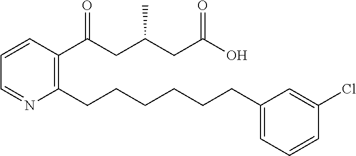 [SMILES: C[C@H](CC(=O)O)CC(=O)c1cccnc1CCCCCCc1cccc(Cl)c1] |
| I-95 | 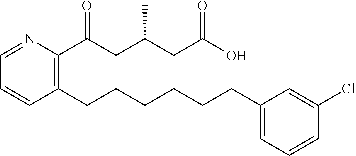 [SMILES: C[C@H](CC(=O)O)CC(=O)c1ncccc1CCCCCCc1cccc(Cl)c1] |
| I-96 | 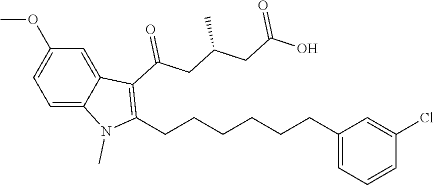 [SMILES: COc1ccc2c(c1)c(C(=O)C[C@H](C)CC(=O)O)c(CCCCCCc1cccc(Cl)c1)n2C] |
| I-97 | 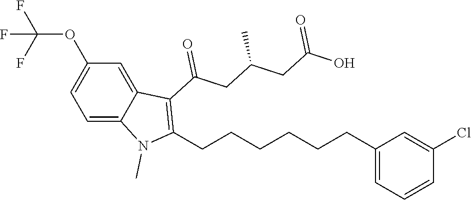 [SMILES: C[C@H](CC(=O)O)CC(=O)c1c(CCCCCCc2cccc(Cl)c2)n(C)c2ccc(OC(F)(F)F)cc12] |
| I-98 | 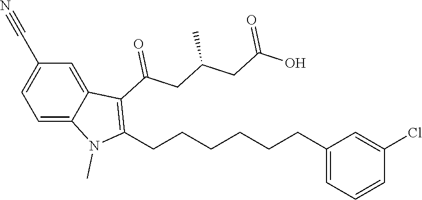 [SMILES: C[C@H](CC(=O)O)CC(=O)c1c(CCCCCCc2cccc(Cl)c2)n(C)c2ccc(C#N)cc12] |
| I-99 | 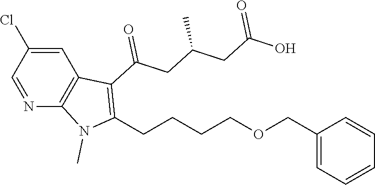 [SMILES: C[C@H](CC(=O)O)CC(=O)c1c(CCCCOCc2ccccc2)n(C)c2ncc(Cl)cc12] |
| I-100 | 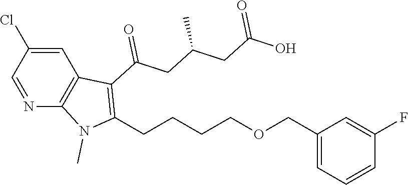 [SMILES: C[C@H](CC(=O)O)CC(=O)c1c(CCCCOCc2cccc(F)c2)n(C)c2ncc(Cl)cc12] |
| I-101 | 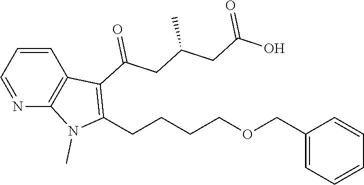 [SMILES: C[C@H](CC(=O)O)CC(=O)c1c(CCCCOCc2ccccc2)n(C)c2ncccc12] |
| I-102 | 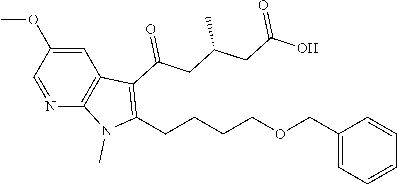 [SMILES: COc1cnc2c(c1)c(C(=O)C[C@H](C)CC(=O)O)c(CCCCOCc1ccccc1)n2C] |
| I-103 | 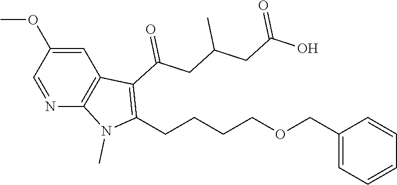 [SMILES: COc1cnc2c(c1)c(C(=O)CC(C)CC(=O)O)c(CCCCOCc1ccccc1)n2C] |
| I-104 | 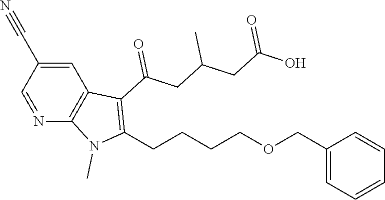 [SMILES: CC(CC(=O)O)CC(=O)c1c(CCCCOCc2ccccc2)n(C)c2ncc(C#N)cc12] |
| I-105 | 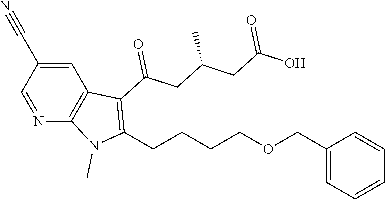 [SMILES: C[C@H](CC(=O)O)CC(=O)c1c(CCCCOCc2ccccc2)n(C)c2ncc(C#N)cc12] |
| I-106 | 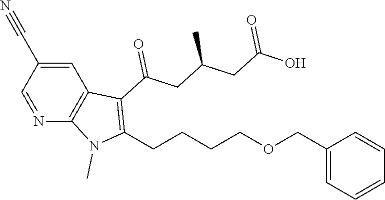 [SMILES: C[C@@H](CC(=O)O)CC(=O)c1c(CCCCOCc2ccccc2)n(C)c2ncc(C#N)cc12] |
| I-107 | 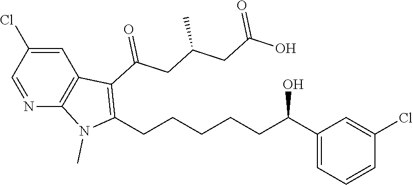 [SMILES: C[C@H](CC(=O)O)CC(=O)c1c(CCCCC[C@@H](O)c2cccc(Cl)c2)n(C)c2ncc(Cl)cc12] |
| I-108 | 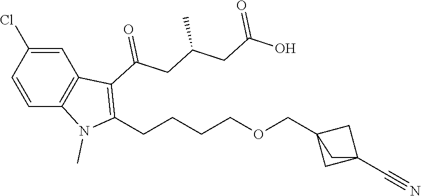 [SMILES: C[C@H](CC(=O)O)CC(=O)c1c(CCCCOCC23CC(C#N)(C2)C3)n(C)c2ccc(Cl)cc12] |
| I-109 | 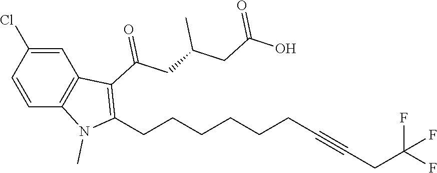 [SMILES: C[C@H](CC(=O)O)CC(=O)c1c(CCCCCCC#CCC(F)(F)F)n(C)c2ccc(Cl)cc12] |
| I-110 | 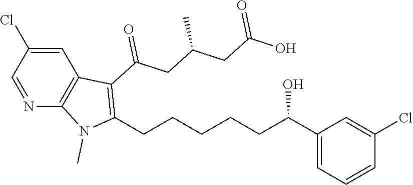 [SMILES: C[C@H](CC(=O)O)CC(=O)c1c(CCCCC[C@H](O)c2cccc(Cl)c2)n(C)c2ncc(Cl)cc12] |
| I-111 | 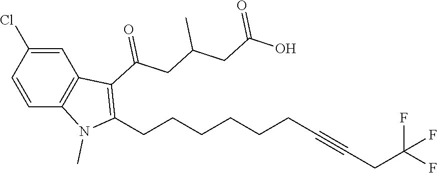 [SMILES: CC(CC(=O)O)CC(=O)c1c(CCCCCCC#CCC(F)(F)F)n(C)c2ccc(Cl)cc12] |
| I-112 | 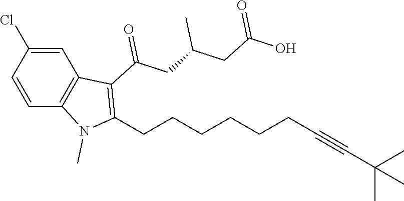 [SMILES: C[C@H](CC(=O)O)CC(=O)c1c(CCCCCCC#CC(C)(C)C)n(C)c2ccc(Cl)cc12] |
| I-113 | 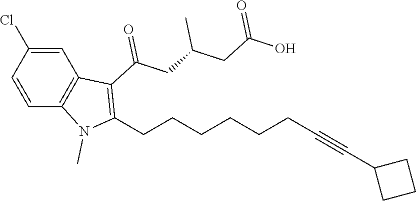 [SMILES: C[C@H](CC(=O)O)CC(=O)c1c(CCCCCCC#CC2CCC2)n(C)c2ccc(Cl)cc12] |
| I-114 | 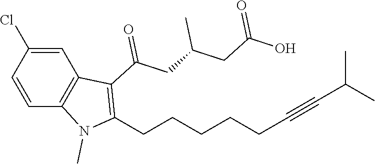 [SMILES: CC(C)C#CCCCCCc1c(C(=O)C[C@H](C)CC(=O)O)c2cc(Cl)ccc2n1C] |
| I-115 | 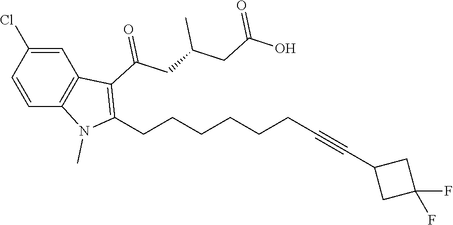 [SMILES: C[C@H](CC(=O)O)CC(=O)c1c(CCCCCCC#CC2CC(F)(F)C2)n(C)c2ccc(Cl)cc12] |
| I-116 | 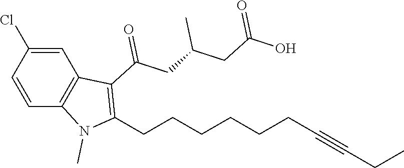 [SMILES: CCC#CCCCCCCc1c(C(=O)C[C@H](C)CC(=O)O)c2cc(Cl)ccc2n1C] |
| I-117 | 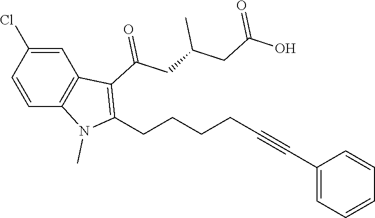 [SMILES: C[C@H](CC(=O)O)CC(=O)c1c(CCCCC#Cc2ccccc2)n(C)c2ccc(Cl)cc12] |
| I-118 | 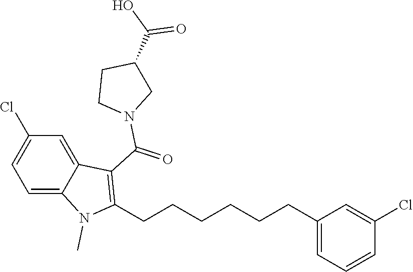 [SMILES: Cn1c(CCCCCCc2cccc(Cl)c2)c(C(=O)N2CC[C@H](C(=O)O)C2)c2cc(Cl)ccc21] |
| I-119 | 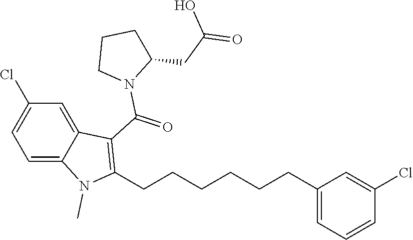 [SMILES: Cn1c(CCCCCCc2cccc(Cl)c2)c(C(=O)N2CCC[C@@H]2CC(=O)O)c2cc(Cl)ccc21] |
| I-120 | 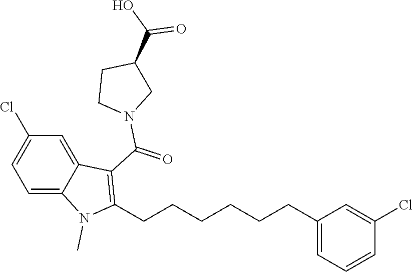 [SMILES: Cn1c(CCCCCCc2cccc(Cl)c2)c(C(=O)N2CC[C@@H](C(=O)O)C2)c2cc(Cl)ccc21] |
| I-121 | 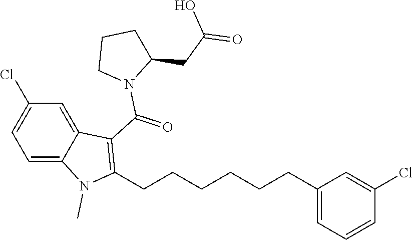 [SMILES: Cn1c(CCCCCCc2cccc(Cl)c2)c(C(=O)N2CCC[C@H]2CC(=O)O)c2cc(Cl)ccc21] |
| I-122 | 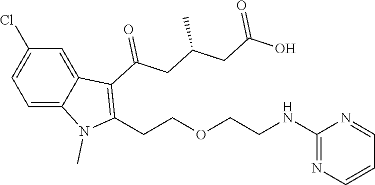 [SMILES: C[C@H](CC(=O)O)CC(=O)c1c(CCOCCNc2ncccn2)n(C)c2ccc(Cl)cc12] |
| I-123 | 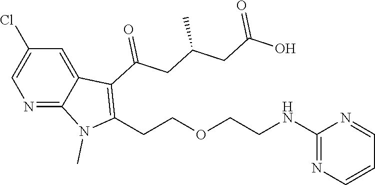 [SMILES: C[C@H](CC(=O)O)CC(=O)c1c(CCOCCNc2ncccn2)n(C)c2ncc(Cl)cc12] |
| I-124 | 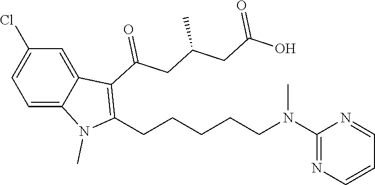 [SMILES: C[C@H](CC(=O)O)CC(=O)c1c(CCCCCN(C)c2ncccn2)n(C)c2ccc(Cl)cc12] |
| I-125 | 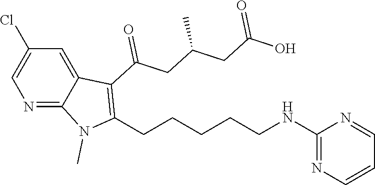 [SMILES: C[C@H](CC(=O)O)CC(=O)c1c(CCCCCNc2ncccn2)n(C)c2ncc(Cl)cc12] |
| I-126 | 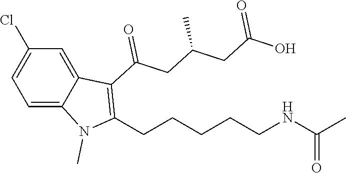 [SMILES: CC(=O)NCCCCCc1c(C(=O)C[C@H](C)CC(=O)O)c2cc(Cl)ccc2n1C] |
| I-127 | 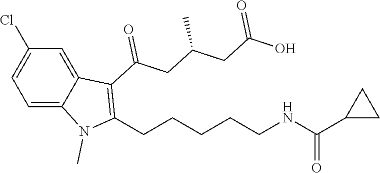 [SMILES: C[C@H](CC(=O)O)CC(=O)c1c(CCCCCNC(=O)C2CC2)n(C)c2ccc(Cl)cc12] |
| I-128 | 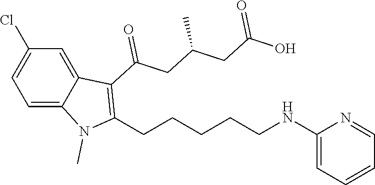 [SMILES: C[C@H](CC(=O)O)CC(=O)c1c(CCCCCNc2ccccn2)n(C)c2ccc(Cl)cc12] |
| I-129 | 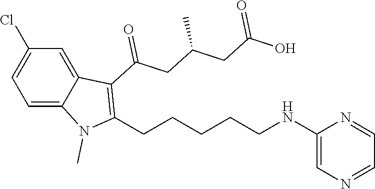 [SMILES: C[C@H](CC(=O)O)CC(=O)c1c(CCCCCNc2cnccn2)n(C)c2ccc(Cl)cc12] |
| I-130 | 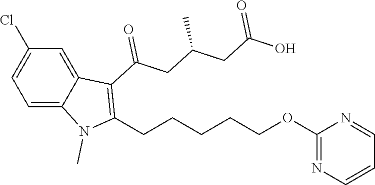 [SMILES: C[C@H](CC(=O)O)CC(=O)c1c(CCCCCOc2ncccn2)n(C)c2ccc(Cl)cc12] |
| I-131 | 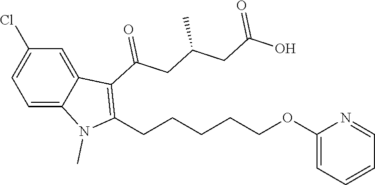 [SMILES: C[C@H](CC(=O)O)CC(=O)c1c(CCCCCOc2ccccn2)n(C)c2ccc(Cl)cc12] |
| I-132 | 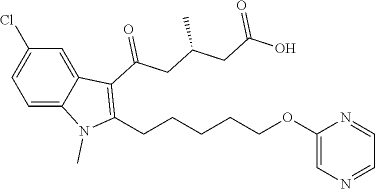 [SMILES: C[C@H](CC(=O)O)CC(=O)c1c(CCCCCOc2cnccn2)n(C)c2ccc(Cl)cc12] |
| I-133 | 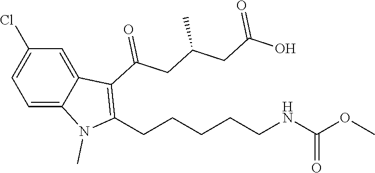 [SMILES: COC(=O)NCCCCCc1c(C(=O)C[C@H](C)CC(=O)O)c2cc(Cl)ccc2n1C] |
| I-134 | 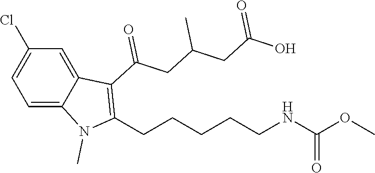 [SMILES: COC(=O)NCCCCCc1c(C(=O)CC(C)CC(=O)O)c2cc(Cl)ccc2n1C] |
| I-135 | 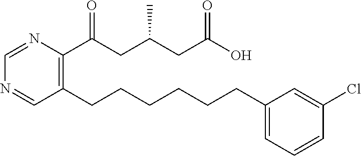 [SMILES: C[C@H](CC(=O)O)CC(=O)c1ncncc1CCCCCCc1cccc(Cl)c1] |
| I-136 | 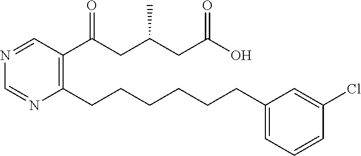 [SMILES: C[C@H](CC(=O)O)CC(=O)c1cncnc1CCCCCCc1cccc(Cl)c1] |
| I-137 | 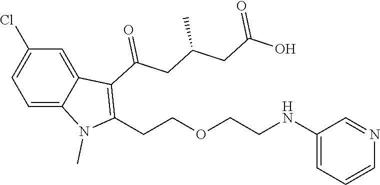 [SMILES: C[C@H](CC(=O)O)CC(=O)c1c(CCOCCNc2cccnc2)n(C)c2ccc(Cl)cc12] |
| I-138 | 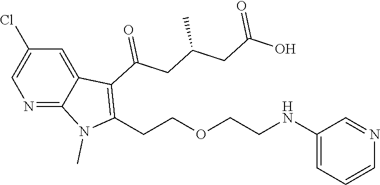 [SMILES: C[C@H](CC(=O)O)CC(=O)c1c(CCOCCNc2cccnc2)n(C)c2ncc(Cl)cc12] |
| I-139 | 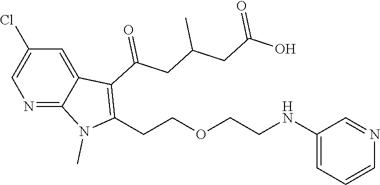 [SMILES: CC(CC(=O)O)CC(=O)c1c(CCOCCNc2cccnc2)n(C)c2ncc(Cl)cc12] |
| I-140 | 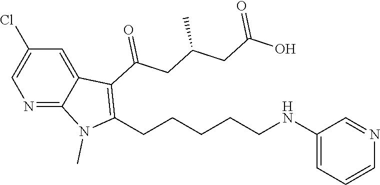 [SMILES: C[C@H](CC(=O)O)CC(=O)c1c(CCCCCNc2cccnc2)n(C)c2ncc(Cl)cc12] |
| I-141 | 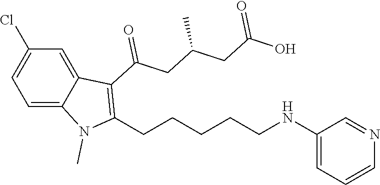 [SMILES: C[C@H](CC(=O)O)CC(=O)c1c(CCCCCNc2cccnc2)n(C)c2ccc(Cl)cc12] |
| I-142 | 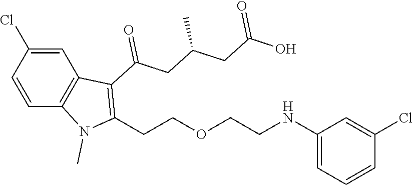 [SMILES: C[C@H](CC(=O)O)CC(=O)c1c(CCOCCNc2cccc(Cl)c2)n(C)c2ccc(Cl)cc12] |
| I-143 | 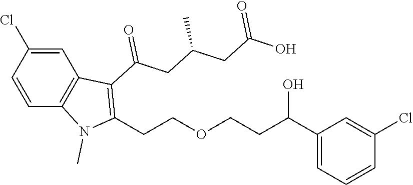 [SMILES: C[C@H](CC(=O)O)CC(=O)c1c(CCOCCC(O)c2cccc(Cl)c2)n(C)c2ccc(Cl)cc12] |
| I-144 | 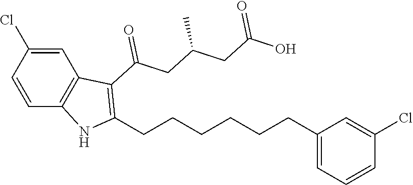 [SMILES: C[C@H](CC(=O)O)CC(=O)c1c(CCCCCCc2cccc(Cl)c2)[nH]c2ccc(Cl)cc12] |
| I-145 | 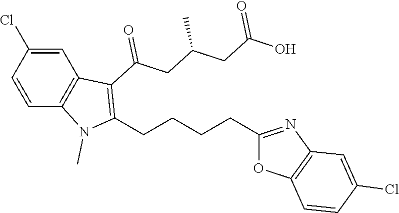 [SMILES: C[C@H](CC(=O)O)CC(=O)c1c(CCCCc2nc3cc(Cl)ccc3o2)n(C)c2ccc(Cl)cc12] |
| I-146 | 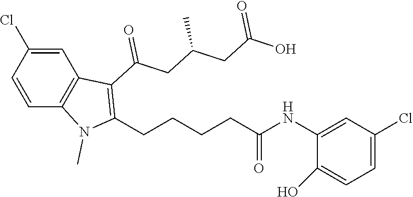 [SMILES: C[C@H](CC(=O)O)CC(=O)c1c(CCCCC(=O)Nc2cc(Cl)ccc2O)n(C)c2ccc(Cl)cc12] |
| I-147 | 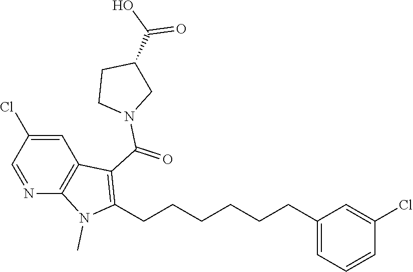 [SMILES: Cn1c(CCCCCCc2cccc(Cl)c2)c(C(=O)N2CC[C@H](C(=O)O)C2)c2cc(Cl)cnc21] |
| I-148 | 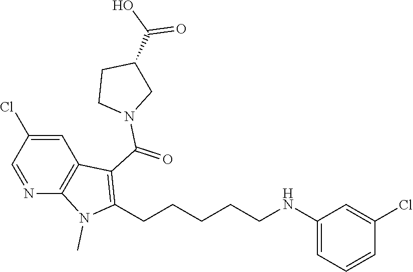 [SMILES: Cn1c(CCCCCNc2cccc(Cl)c2)c(C(=O)N2CC[C@H](C(=O)O)C2)c2cc(Cl)cnc21] |
| I-149 | 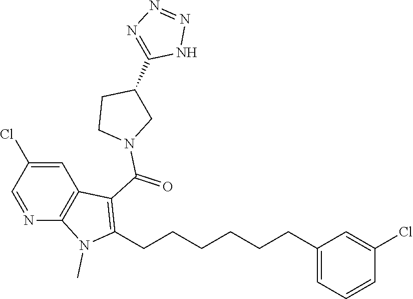 [SMILES: Cn1c(CCCCCCc2cccc(Cl)c2)c(C(=O)N2CC[C@H](c3nnn[nH]3)C2)c2cc(Cl)cnc21] |
| I-150 | 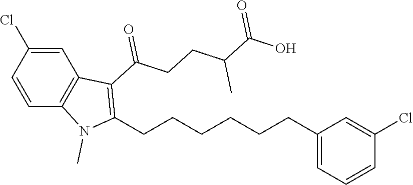 [SMILES: CC(CCC(=O)c1c(CCCCCCc2cccc(Cl)c2)n(C)c2ccc(Cl)cc12)C(=O)O] |
| I-151 | 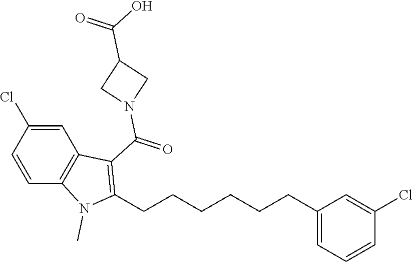 [SMILES: Cn1c(CCCCCCc2cccc(Cl)c2)c(C(=O)N2CC(C(=O)O)C2)c2cc(Cl)ccc21] |
| I-152 | 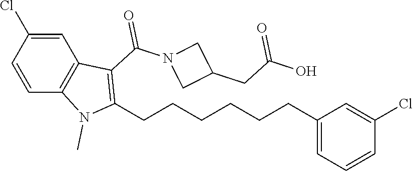 [SMILES: Cn1c(CCCCCCc2cccc(Cl)c2)c(C(=O)N2CC(CC(=O)O)C2)c2cc(Cl)ccc21] |
| I-153 | 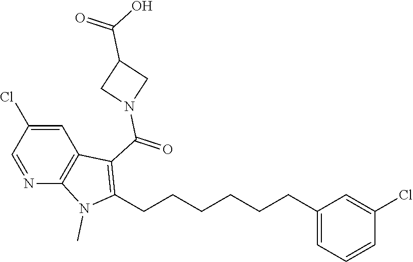 [SMILES: Cn1c(CCCCCCc2cccc(Cl)c2)c(C(=O)N2CC(C(=O)O)C2)c2cc(Cl)cnc21] |
| I-154 | 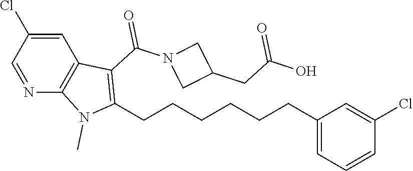 [SMILES: Cn1c(CCCCCCc2cccc(Cl)c2)c(C(=O)N2CC(CC(=O)O)C2)c2cc(Cl)cnc21] |
| I-155 | 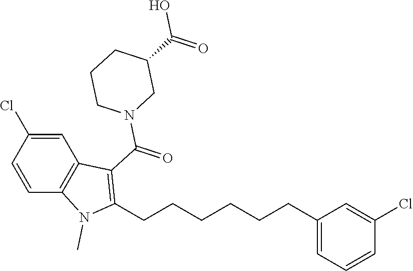 [SMILES: Cn1c(CCCCCCc2cccc(Cl)c2)c(C(=O)N2CCC[C@H](C(=O)O)C2)c2cc(Cl)ccc21] |
| I-156 | 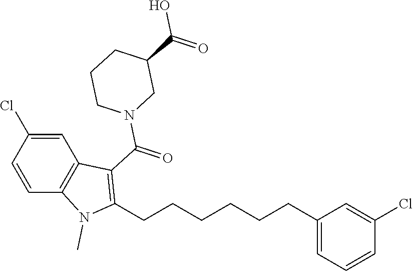 [SMILES: Cn1c(CCCCCCc2cccc(Cl)c2)c(C(=O)N2CCC[C@@H](C(=O)O)C2)c2cc(Cl)ccc21] |
| I-157 | 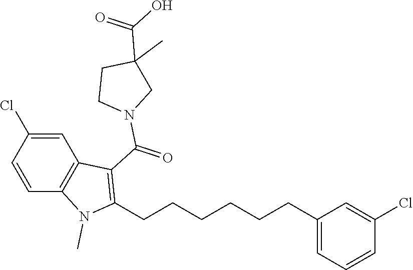 [SMILES: Cn1c(CCCCCCc2cccc(Cl)c2)c(C(=O)N2CCC(C)(C(=O)O)C2)c2cc(Cl)ccc21] |
| I-158 | 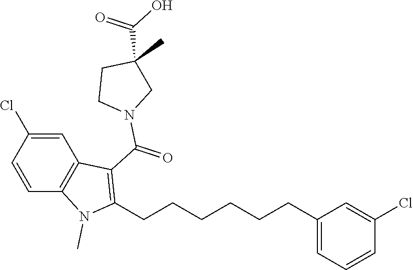 [SMILES: Cn1c(CCCCCCc2cccc(Cl)c2)c(C(=O)N2CC[C@](C)(C(=O)O)C2)c2cc(Cl)ccc21] |
| I-159 | 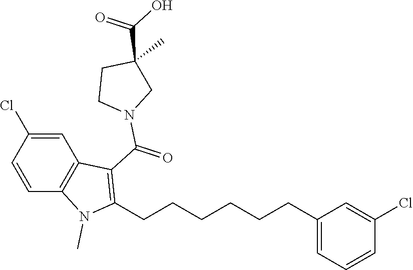 [SMILES: Cn1c(CCCCCCc2cccc(Cl)c2)c(C(=O)N2CC[C@@](C)(C(=O)O)C2)c2cc(Cl)ccc21] |
| I-160 | 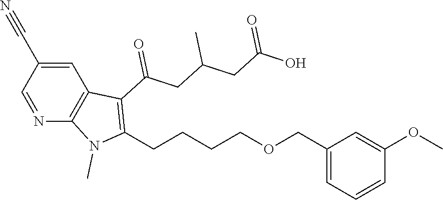 [SMILES: COc1cccc(COCCCCc2c(C(=O)CC(C)CC(=O)O)c3cc(C#N)cnc3n2C)c1] |
| I-161 | 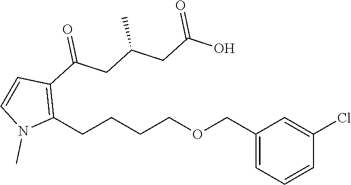 [SMILES: C[C@H](CC(=O)O)CC(=O)c1ccn(C)c1CCCCOCc1cccc(Cl)c1] |
| I-162 | 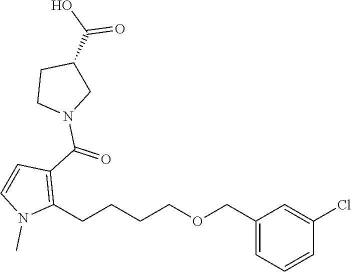 [SMILES: Cn1ccc(C(=O)N2CC[C@H](C(=O)O)C2)c1CCCCOCc1cccc(Cl)c1] |
| I-163 | 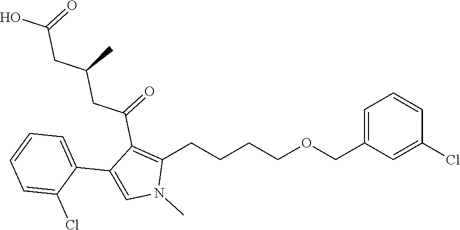 [SMILES: C[C@H](CC(=O)O)CC(=O)c1c(-c2ccccc2Cl)cn(C)c1CCCCOCc1cccc(Cl)c1] |
| I-164 | 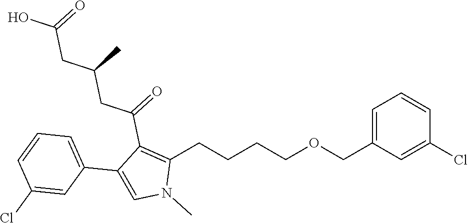 [SMILES: C[C@H](CC(=O)O)CC(=O)c1c(-c2cccc(Cl)c2)cn(C)c1CCCCOCc1cccc(Cl)c1] |
| I-165 | 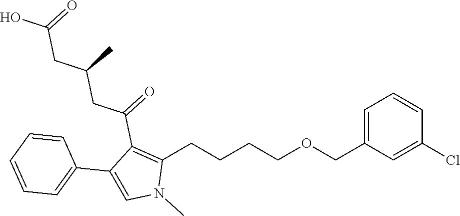 [SMILES: C[C@H](CC(=O)O)CC(=O)c1c(-c2ccccc2)cn(C)c1CCCCOCc1cccc(Cl)c1] |
| I-166 | 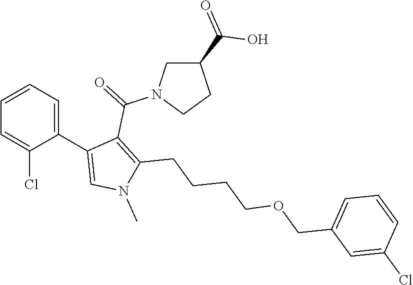 [SMILES: Cn1cc(-c2ccccc2Cl)c(C(=O)N2CC[C@H](C(=O)O)C2)c1CCCCOCc1cccc(Cl)c1] |
| I-167 | 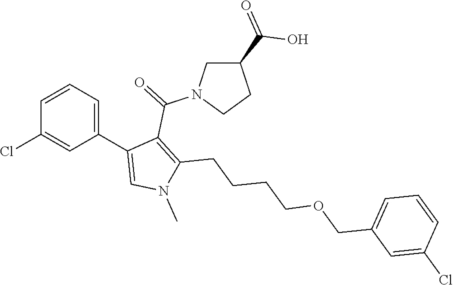 [SMILES: Cn1cc(-c2cccc(Cl)c2)c(C(=O)N2CC[C@H](C(=O)O)C2)c1CCCCOCc1cccc(Cl)c1] |
| I-168 | 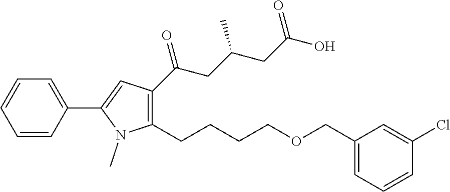 [SMILES: C[C@H](CC(=O)O)CC(=O)c1cc(-c2ccccc2)n(C)c1CCCCOCc1cccc(Cl)c1] |
| I-169 | 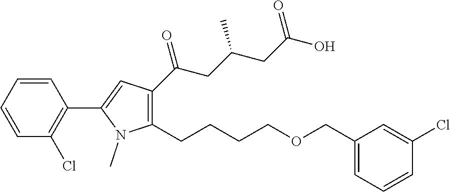 [SMILES: C[C@H](CC(=O)O)CC(=O)c1cc(-c2ccccc2Cl)n(C)c1CCCCOCc1cccc(Cl)c1] |
| I-170 | 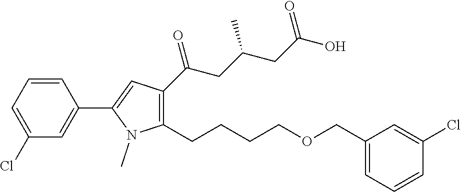 [SMILES: C[C@H](CC(=O)O)CC(=O)c1cc(-c2cccc(Cl)c2)n(C)c1CCCCOCc1cccc(Cl)c1] |
| I-171 | 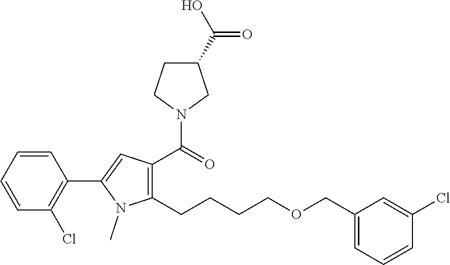 [SMILES: Cn1c(-c2ccccc2Cl)cc(C(=O)N2CC[C@H](C(=O)O)C2)c1CCCCOCc1cccc(Cl)c1] |
| I-172 | 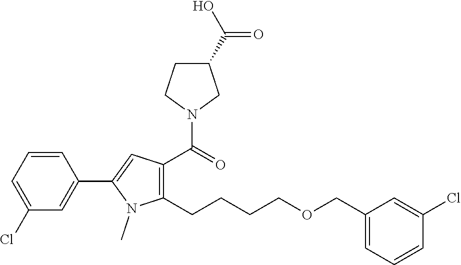 [SMILES: Cn1c(-c2cccc(Cl)c2)cc(C(=O)N2CC[C@H](C(=O)O)C2)c1CCCCOCc1cccc(Cl)c1] |
| I-173 | 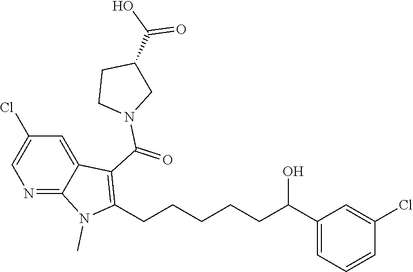 [SMILES: Cn1c(CCCCCC(O)c2cccc(Cl)c2)c(C(=O)N2CC[C@H](C(=O)O)C2)c2cc(Cl)cnc21] |
| I-174 | 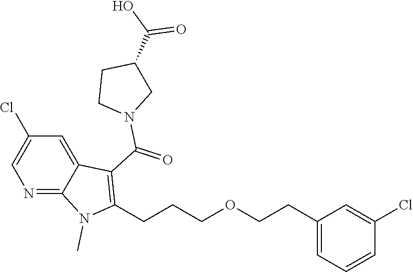 [SMILES: Cn1c(CCCOCCc2cccc(Cl)c2)c(C(=O)N2CC[C@H](C(=O)O)C2)c2cc(Cl)cnc21] |
| I-175 | 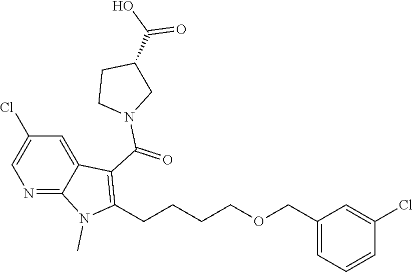 [SMILES: Cn1c(CCCCOCc2cccc(Cl)c2)c(C(=O)N2CC[C@H](C(=O)O)C2)c2cc(Cl)cnc21] |
| I-176 | 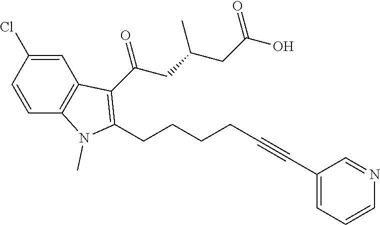 [SMILES: C[C@H](CC(=O)O)CC(=O)c1c(CCCCC#Cc2cccnc2)n(C)c2ccc(Cl)cc12] |
| I-177 | 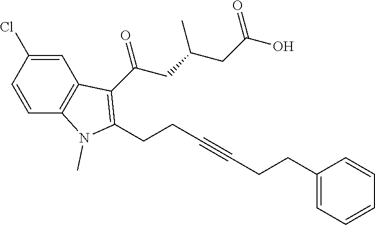 [SMILES: C[C@H](CC(=O)O)CC(=O)c1c(CCC#CCCc2ccccc2)n(C)c2ccc(Cl)cc12] |
| I-178 | 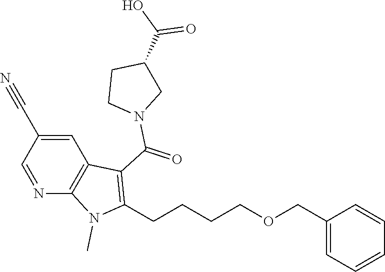 [SMILES: Cn1c(CCCCOCc2ccccc2)c(C(=O)N2CC[C@H](C(=O)O)C2)c2cc(C#N)cnc21] |
| I-179 | 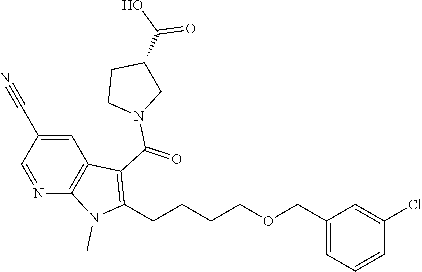 [SMILES: Cn1c(CCCCOCc2cccc(Cl)c2)c(C(=O)N2CC[C@H](C(=O)O)C2)c2cc(C#N)cnc21] |
| I-180 | 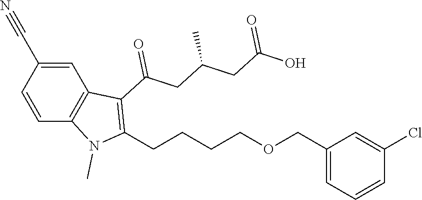 [SMILES: C[C@H](CC(=O)O)CC(=O)c1c(CCCCOCc2cccc(Cl)c2)n(C)c2ccc(C#N)cc12] |
| I-181 | 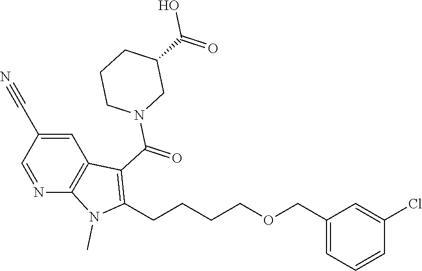 [SMILES: Cn1c(CCCCOCc2cccc(Cl)c2)c(C(=O)N2CCC[C@H](C(=O)O)C2)c2cc(C#N)cnc21] |
| I-182 | 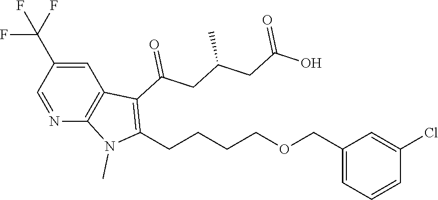 [SMILES: C[C@H](CC(=O)O)CC(=O)c1c(CCCCOCc2cccc(Cl)c2)n(C)c2ncc(C(F)(F)F)cc12] |
| I-183 | 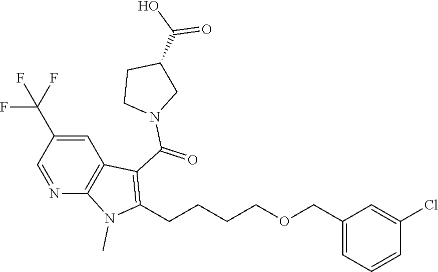 [SMILES: Cn1c(CCCCOCc2cccc(Cl)c2)c(C(=O)N2CC[C@H](C(=O)O)C2)c2cc(C(F)(F)F)cnc21] |
| I-184 | 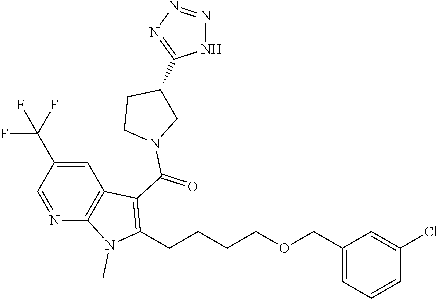 [SMILES: Cn1c(CCCCOCc2cccc(Cl)c2)c(C(=O)N2CC[C@H](c3nnn[nH]3)C2)c2cc(C(F)(F)F)cnc21] |
| I-185 | 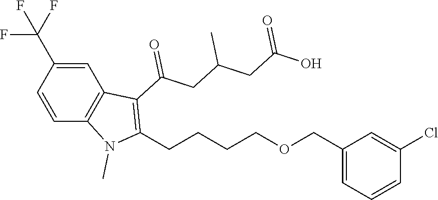 [SMILES: CC(CC(=O)O)CC(=O)c1c(CCCCOCc2cccc(Cl)c2)n(C)c2ccc(C(F)(F)F)cc12] |
| I-186 | 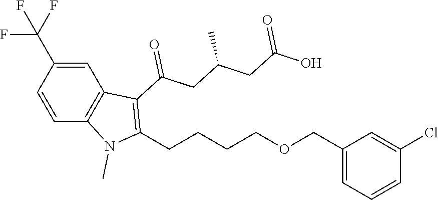 [SMILES: C[C@H](CC(=O)O)CC(=O)c1c(CCCCOCc2cccc(Cl)c2)n(C)c2ccc(C(F)(F)F)cc12] |
| I-187 | 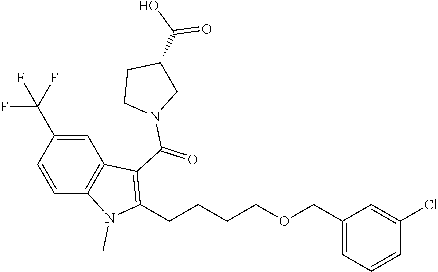 [SMILES: Cn1c(CCCCOCc2cccc(Cl)c2)c(C(=O)N2CC[C@H](C(=O)O)C2)c2cc(C(F)(F)F)ccc21] |
| I-188 | 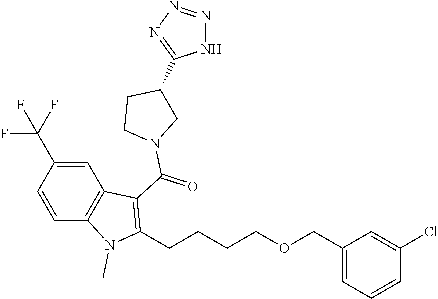 [SMILES: Cn1c(CCCCOCc2cccc(Cl)c2)c(C(=O)N2CC[C@H](c3nnn[nH]3)C2)c2cc(C(F)(F)F)ccc21] |
| I-189 | 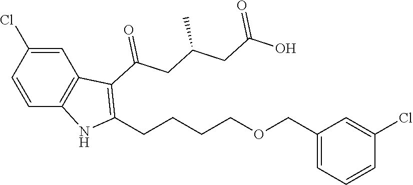 [SMILES: C[C@H](CC(=O)O)CC(=O)c1c(CCCCOCc2cccc(Cl)c2)[nH]c2ccc(Cl)cc12] |
| I-190 | 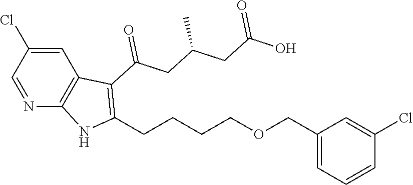 [SMILES: C[C@H](CC(=O)O)CC(=O)c1c(CCCCOCc2cccc(Cl)c2)[nH]c2ncc(Cl)cc12] |
| I-191 | 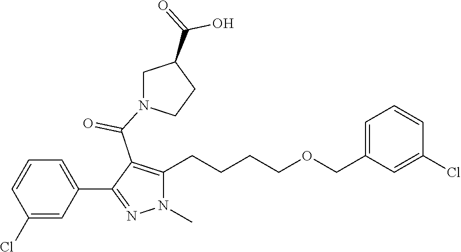 [SMILES: Cn1nc(-c2cccc(Cl)c2)c(C(=O)N2CC[C@H](C(=O)O)C2)c1CCCCOCc1cccc(Cl)c1] |
| I-192 | 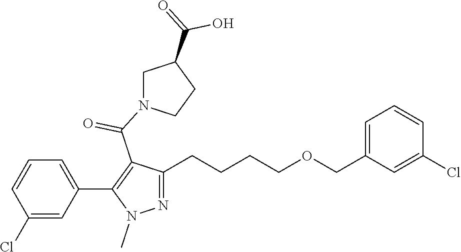 [SMILES: Cn1nc(CCCCOCc2cccc(Cl)c2)c(C(=O)N2CC[C@H](C(=O)O)C2)c1-c1cccc(Cl)c1] |
| I-193 | 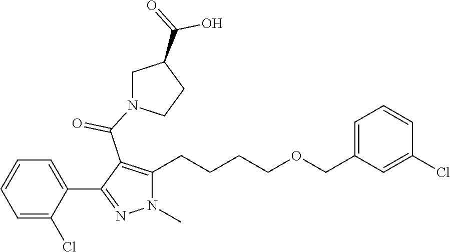 [SMILES: Cn1nc(-c2ccccc2Cl)c(C(=O)N2CC[C@H](C(=O)O)C2)c1CCCCOCc1cccc(Cl)c1] |
| I-194 | 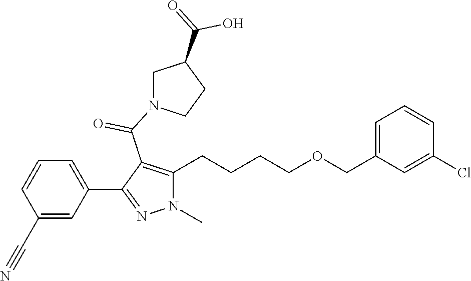 [SMILES: Cn1nc(-c2cccc(C#N)c2)c(C(=O)N2CC[C@H](C(=O)O)C2)c1CCCCOCc1cccc(Cl)c1] |
| I-195 | 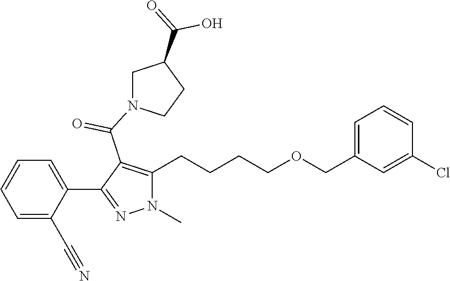 [SMILES: Cn1nc(-c2ccccc2C#N)c(C(=O)N2CC[C@H](C(=O)O)C2)c1CCCCOCc1cccc(Cl)c1] |
| I-196 | 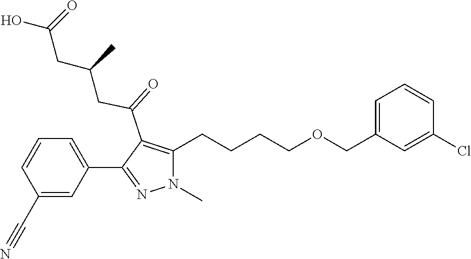 [SMILES: C[C@H](CC(=O)O)CC(=O)c1c(-c2cccc(C#N)c2)nn(C)c1CCCCOCc1cccc(Cl)c1] |
| I-197 | 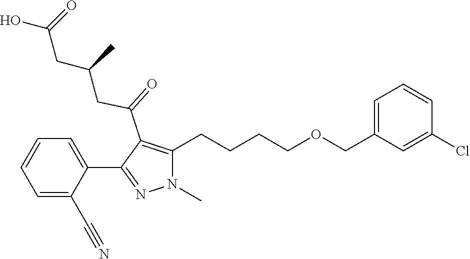 [SMILES: C[C@H](CC(=O)O)CC(=O)c1c(-c2ccccc2C#N)nn(C)c1CCCCOCc1cccc(Cl)c1] |
| I-198 | 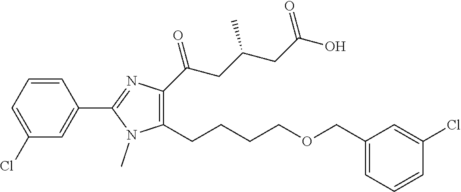 [SMILES: C[C@H](CC(=O)O)CC(=O)c1nc(-c2cccc(Cl)c2)n(C)c1CCCCOCc1cccc(Cl)c1] |
| I-199 | 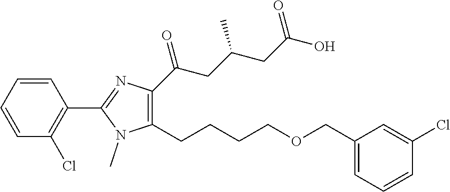 [SMILES: C[C@H](CC(=O)O)CC(=O)c1nc(-c2ccccc2Cl)n(C)c1CCCCOCc1cccc(Cl)c1] |
| I-200 | 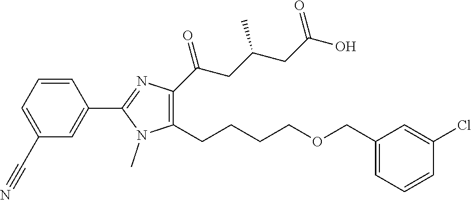 [SMILES: C[C@H](CC(=O)O)CC(=O)c1nc(-c2cccc(C#N)c2)n(C)c1CCCCOCc1cccc(Cl)c1] |
| I-201 | 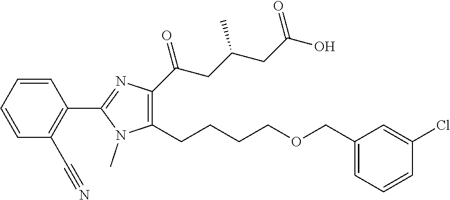 [SMILES: C[C@H](CC(=O)O)CC(=O)c1nc(-c2ccccc2C#N)n(C)c1CCCCOCc1cccc(Cl)c1] |
| I-202 | 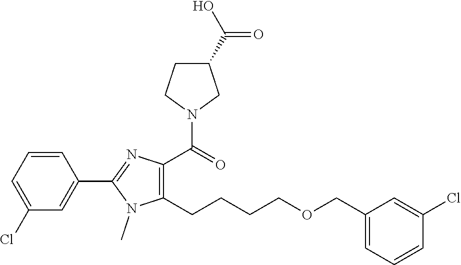 [SMILES: Cn1c(-c2cccc(Cl)c2)nc(C(=O)N2CC[C@H](C(=O)O)C2)c1CCCCOCc1cccc(Cl)c1] |
| I-203 | 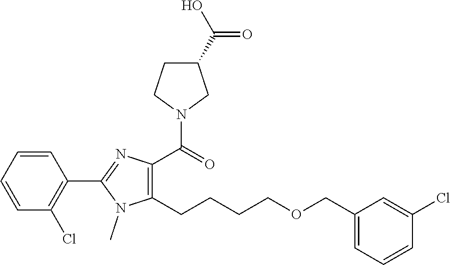 [SMILES: Cn1c(-c2ccccc2Cl)nc(C(=O)N2CC[C@H](C(=O)O)C2)c1CCCCOCc1cccc(Cl)c1] |
| I-204 | 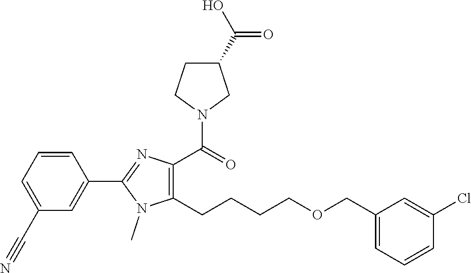 [SMILES: Cn1c(-c2cccc(C#N)c2)nc(C(=O)N2CC[C@H](C(=O)O)C2)c1CCCCOCc1cccc(Cl)c1] |
| I-205 | 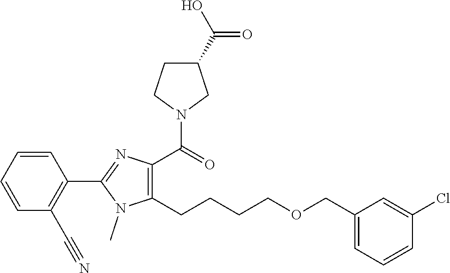 [SMILES: Cn1c(-c2ccccc2C#N)nc(C(=O)N2CC[C@H](C(=O)O)C2)c1CCCCOCc1cccc(Cl)c1] |
| I-206 | 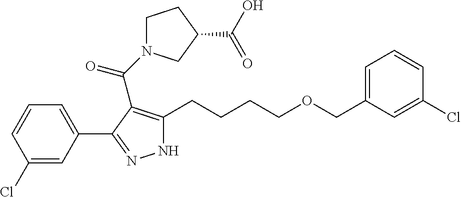 [SMILES: O=C(O)[C@H]1CCN(C(=O)c2c(-c3cccc(Cl)c3)n[nH]c2CCCCOCc2cccc(Cl)c2)C1] |
| I-207 | 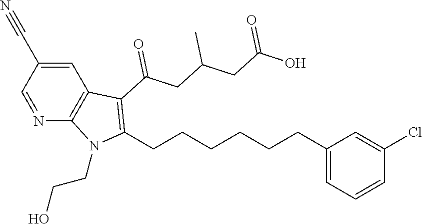 [SMILES: CC(CC(=O)O)CC(=O)c1c(CCCCCCc2cccc(Cl)c2)n(CCO)c2ncc(C#N)cc12] |
| I-208 | 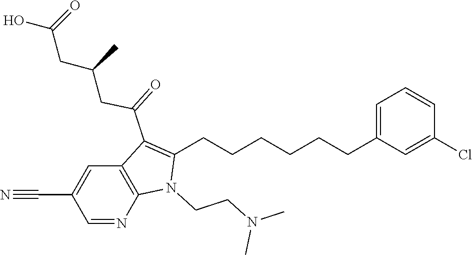 [SMILES: C[C@H](CC(=O)O)CC(=O)c1c(CCCCCCc2cccc(Cl)c2)n(CCN(C)C)c2ncc(C#N)cc12] |
| I-209 | 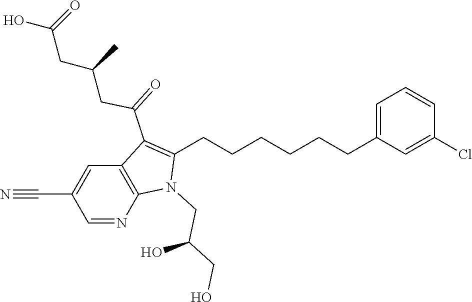 [SMILES: C[C@H](CC(=O)O)CC(=O)c1c(CCCCCCc2cccc(Cl)c2)n(C[C@H](O)CO)c2ncc(C#N)cc12] |
| I-210 | 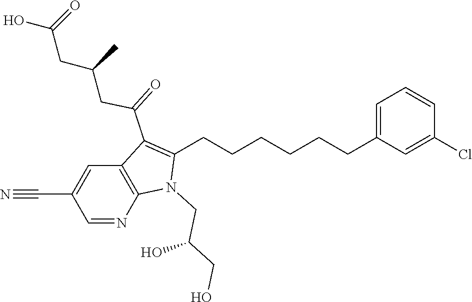 [SMILES: C[C@H](CC(=O)O)CC(=O)c1c(CCCCCCc2cccc(Cl)c2)n(C[C@@H](O)CO)c2ncc(C#N)cc12] |
| I-211 | 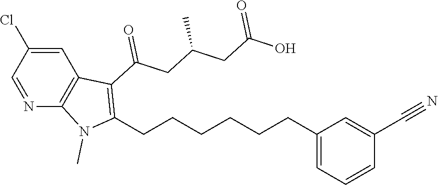 [SMILES: C[C@H](CC(=O)O)CC(=O)c1c(CCCCCCc2cccc(C#N)c2)n(C)c2ncc(Cl)cc12] |
| I-212 | 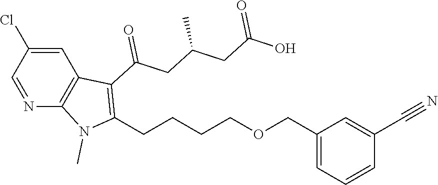 [SMILES: C[C@H](CC(=O)O)CC(=O)c1c(CCCCOCc2cccc(C#N)c2)n(C)c2ncc(Cl)cc12] |
| I-213 | 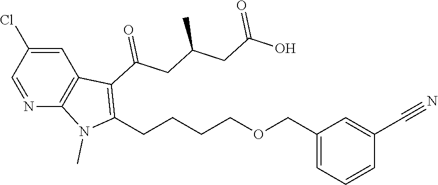 [SMILES: C[C@@H](CC(=O)O)CC(=O)c1c(CCCCOCc2cccc(C#N)c2)n(C)c2ncc(Cl)cc12] |
| I-214 | 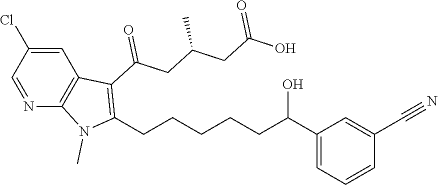 [SMILES: C[C@H](CC(=O)O)CC(=O)c1c(CCCCCC(O)c2cccc(C#N)c2)n(C)c2ncc(Cl)cc12] |
| I-215 | 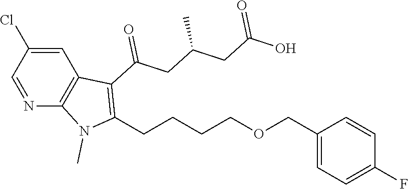 [SMILES: C[C@H](CC(=O)O)CC(=O)c1c(CCCCOCc2ccc(F)cc2)n(C)c2ncc(Cl)cc12] |
| I-216 | 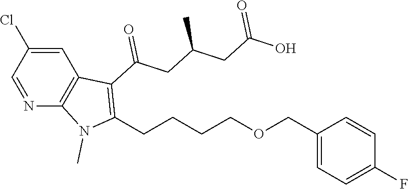 [SMILES: C[C@@H](CC(=O)O)CC(=O)c1c(CCCCOCc2ccc(F)cc2)n(C)c2ncc(Cl)cc12] |
| I-217 | 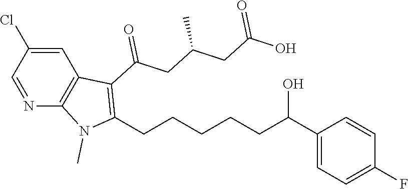 [SMILES: C[C@H](CC(=O)O)CC(=O)c1c(CCCCCC(O)c2ccc(F)cc2)n(C)c2ncc(Cl)cc12] |
| I-218 | 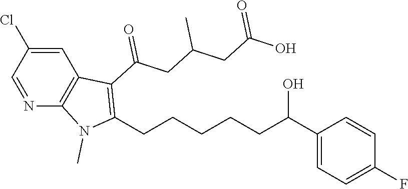 [SMILES: CC(CC(=O)O)CC(=O)c1c(CCCCCC(O)c2ccc(F)cc2)n(C)c2ncc(Cl)cc12] |
| I-219 | 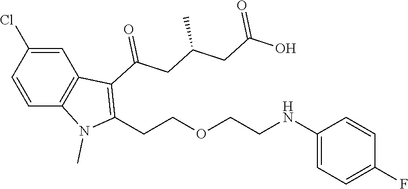 [SMILES: C[C@H](CC(=O)O)CC(=O)c1c(CCOCCNc2ccc(F)cc2)n(C)c2ccc(Cl)cc12] |
| I-220 | 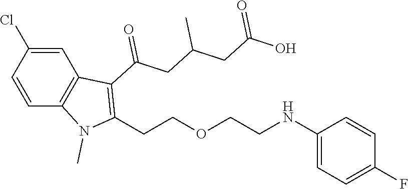 [SMILES: CC(CC(=O)O)CC(=O)c1c(CCOCCNc2ccc(F)cc2)n(C)c2ccc(Cl)cc12] |
| I-221 | 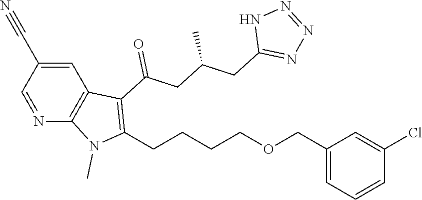 [SMILES: C[C@@H](CC(=O)c1c(CCCCOCc2cccc(Cl)c2)n(C)c2ncc(C#N)cc12)Cc1nnn[nH]1] |
| I-222 | 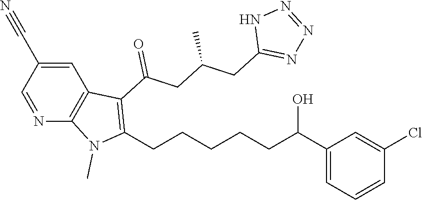 [SMILES: C[C@@H](CC(=O)c1c(CCCCCC(O)c2cccc(Cl)c2)n(C)c2ncc(C#N)cc12)Cc1nnn[nH]1] |
| I-223 | 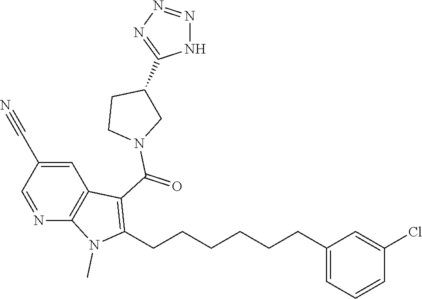 [SMILES: Cn1c(CCCCCCc2cccc(Cl)c2)c(C(=O)N2CC[C@H](c3nnn[nH]3)C2)c2cc(C#N)cnc21] |
| I-224 | 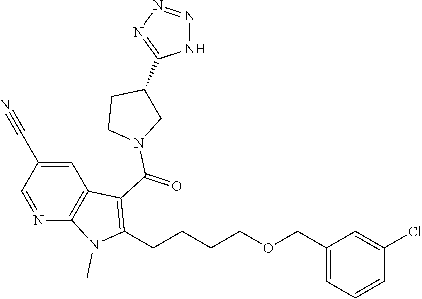 [SMILES: Cn1c(CCCCOCc2cccc(Cl)c2)c(C(=O)N2CC[C@H](c3nnn[nH]3)C2)c2cc(C#N)cnc21] |
| I-225 | 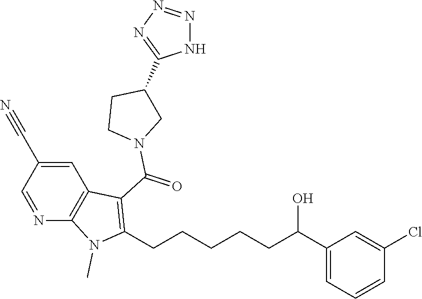 [SMILES: Cn1c(CCCCCC(O)c2cccc(Cl)c2)c(C(=O)N2CC[C@H](c3nnn[nH]3)C2)c2cc(C#N)cnc21] |
| I-226 | 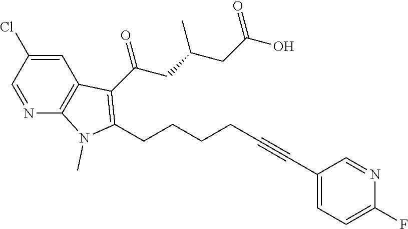 [SMILES: C[C@H](CC(=O)O)CC(=O)c1c(CCCCC#Cc2ccc(F)nc2)n(C)c2ncc(Cl)cc12] |
| I-227 | 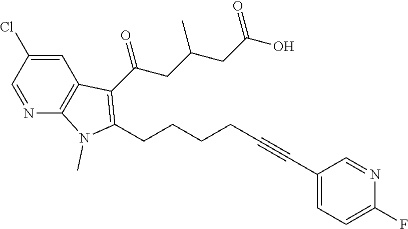 [SMILES: CC(CC(=O)O)CC(=O)c1c(CCCCC#Cc2ccc(F)nc2)n(C)c2ncc(Cl)cc12] |
| I-228 | 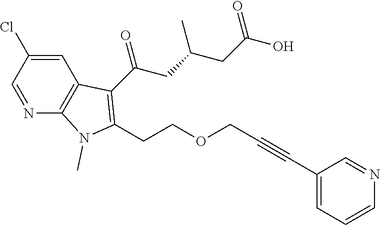 [SMILES: C[C@H](CC(=O)O)CC(=O)c1c(CCOCC#Cc2cccnc2)n(C)c2ncc(Cl)cc12] |
| I-229 | 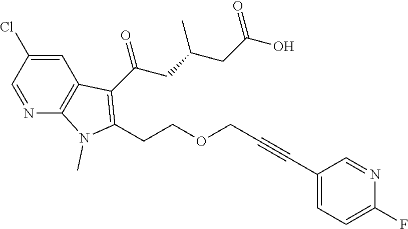 [SMILES: C[C@H](CC(=O)O)CC(=O)c1c(CCOCC#Cc2ccc(F)nc2)n(C)c2ncc(Cl)cc12] |
| I-230 | 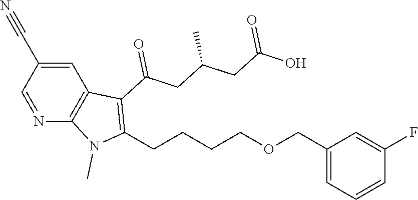 [SMILES: C[C@H](CC(=O)O)CC(=O)c1c(CCCCOCc2cccc(F)c2)n(C)c2ncc(C#N)cc12] |
| I-231 | 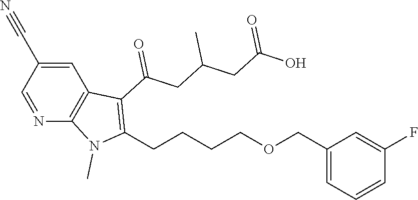 [SMILES: CC(CC(=O)O)CC(=O)c1c(CCCCOCc2cccc(F)c2)n(C)c2ncc(C#N)cc12] |
| I-232 | 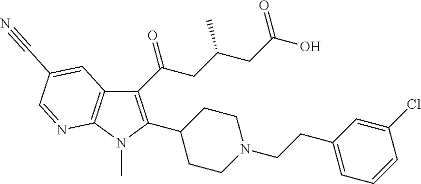 [SMILES: C[C@H](CC(=O)O)CC(=O)c1c(C2CCN(CCc3cccc(Cl)c3)CC2)n(C)c2ncc(C#N)cc12] |
| I-233 | 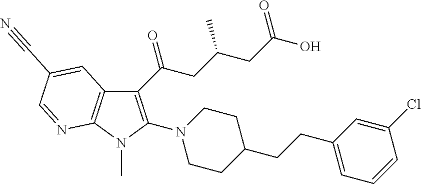 [SMILES: C[C@H](CC(=O)O)CC(=O)c1c(N2CCC(CCc3cccc(Cl)c3)CC2)n(C)c2ncc(C#N)cc12] |
| I-234 | 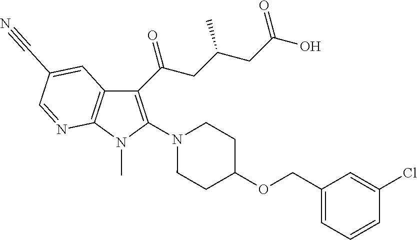 [SMILES: C[C@H](CC(=O)O)CC(=O)c1c(N2CCC(OCc3cccc(Cl)c3)CC2)n(C)c2ncc(C#N)cc12] |
| I-235 | 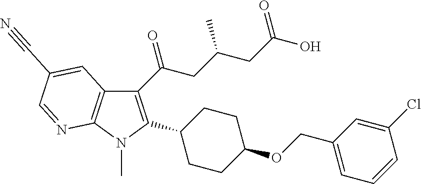 [SMILES: C[C@H](CC(=O)O)CC(=O)c1c2cc(C#N)cnc2n(C)c1[C@H]1CC[C@H](OCc2cccc(Cl)c2)CC1] |
| I-236 | 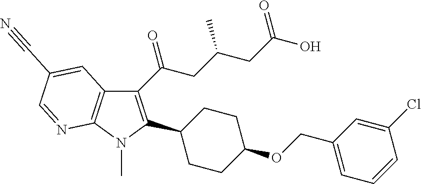 [SMILES: C[C@H](CC(=O)O)CC(=O)c1c2cc(C#N)cnc2n(C)c1[C@H]1CC[C@@H](OCc2cccc(Cl)c2)CC1] |
| I-237 | 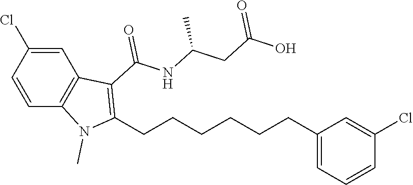 [SMILES: C[C@H](CC(=O)O)NC(=O)c1c(CCCCCCc2cccc(Cl)c2)n(C)c2ccc(Cl)cc12] |
| I-238 | 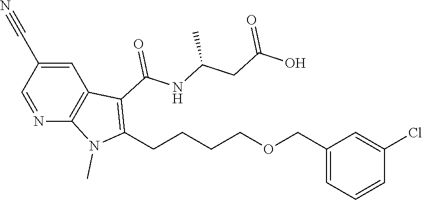 [SMILES: C[C@H](CC(=O)O)NC(=O)c1c(CCCCOCc2cccc(Cl)c2)n(C)c2ncc(C#N)cc12] |
| I-239 | 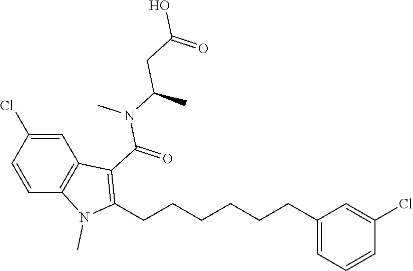 [SMILES: C[C@H](CC(=O)O)N(C)C(=O)c1c(CCCCCCc2cccc(Cl)c2)n(C)c2ccc(Cl)cc12] |
| I-240 | 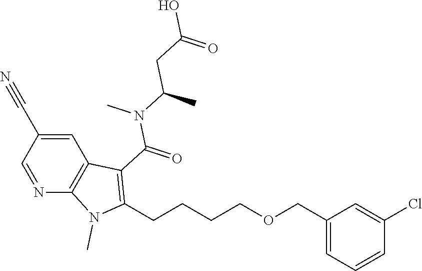 [SMILES: C[C@H](CC(=O)O)N(C)C(=O)c1c(CCCCOCc2cccc(Cl)c2)n(C)c2ncc(C#N)cc12] |
| I-241 | 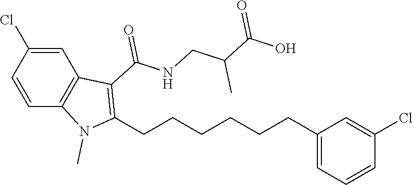 [SMILES: CC(CNC(=O)c1c(CCCCCCc2cccc(Cl)c2)n(C)c2ccc(Cl)cc12)C(=O)O] |
| I-242 | 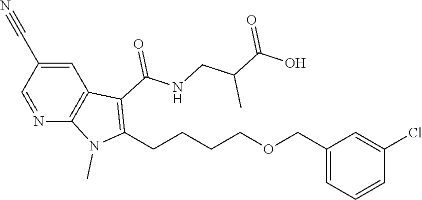 [SMILES: CC(CNC(=O)c1c(CCCCOCc2cccc(Cl)c2)n(C)c2ncc(C#N)cc12)C(=O)O] |
| I-243 | 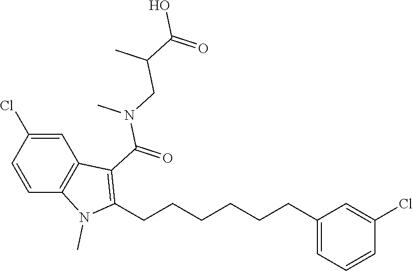 [SMILES: CC(CN(C)C(=O)c1c(CCCCCCc2cccc(Cl)c2)n(C)c2ccc(Cl)cc12)C(=O)O] |
| I-244 | 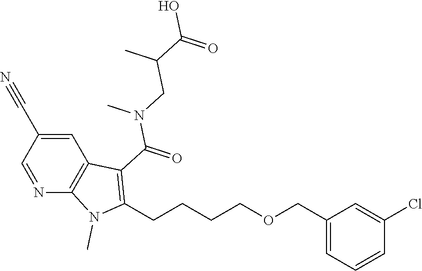 [SMILES: CC(CN(C)C(=O)c1c(CCCCOCc2cccc(Cl)c2)n(C)c2ncc(C#N)cc12)C(=O)O] |
| I-245 | 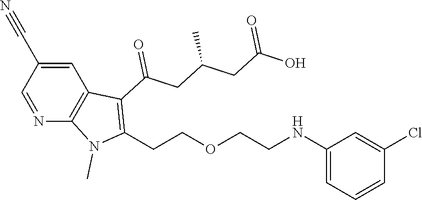 [SMILES: C[C@H](CC(=O)O)CC(=O)c1c(CCOCCNc2cccc(Cl)c2)n(C)c2ncc(C#N)cc12] |
| I-246 | 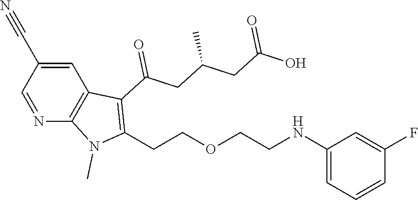 [SMILES: C[C@H](CC(=O)O)CC(=O)c1c(CCOCCNc2cccc(F)c2)n(C)c2ncc(C#N)cc12] |
| I-247 | 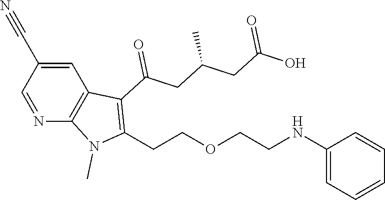 [SMILES: C[C@H](CC(=O)O)CC(=O)c1c(CCOCCNc2ccccc2)n(C)c2ncc(C#N)cc12] |
| I-248 | 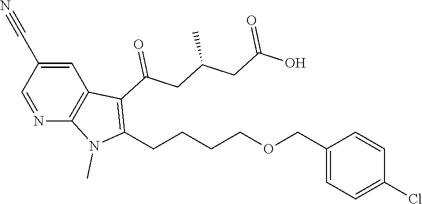 [SMILES: C[C@H](CC(=O)O)CC(=O)c1c(CCCCOCc2ccc(Cl)cc2)n(C)c2ncc(C#N)cc12] |
| I-249 | 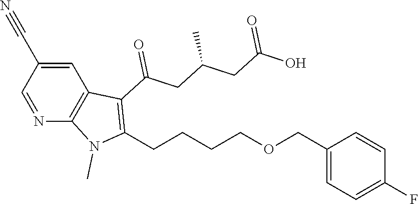 [SMILES: C[C@H](CC(=O)O)CC(=O)c1c(CCCCOCc2ccc(F)cc2)n(C)c2ncc(C#N)cc12] |
| I-250 | 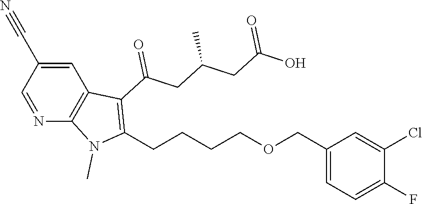 [SMILES: C[C@H](CC(=O)O)CC(=O)c1c(CCCCOCc2ccc(F)c(Cl)c2)n(C)c2ncc(C#N)cc12] |
| I-251 | 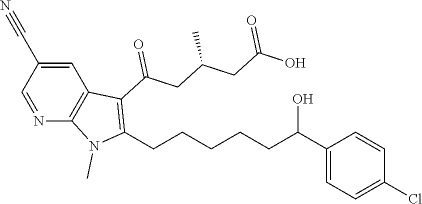 [SMILES: C[C@H](CC(=O)O)CC(=O)c1c(CCCCCC(O)c2ccc(Cl)cc2)n(C)c2ncc(C#N)cc12] |
| I-252 | 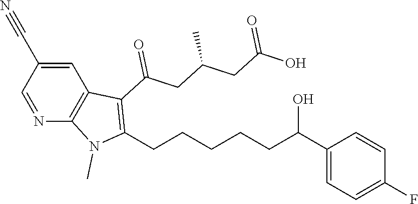 [SMILES: C[C@H](CC(=O)O)CC(=O)c1c(CCCCCC(O)c2ccc(F)cc2)n(C)c2ncc(C#N)cc12] |
| I-253 | 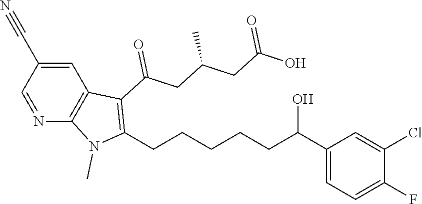 [SMILES: C[C@H](CC(=O)O)CC(=O)c1c(CCCCCC(O)c2ccc(F)c(Cl)c2)n(C)c2ncc(C#N)cc12] |
| I-254 | 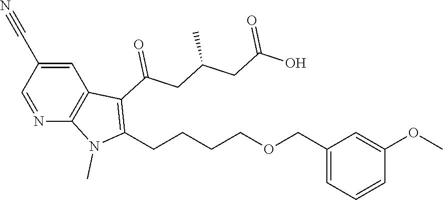 [SMILES: COc1cccc(COCCCCc2c(C(=O)C[C@H](C)CC(=O)O)c3cc(C#N)cnc3n2C)c1] |
| I-255 | 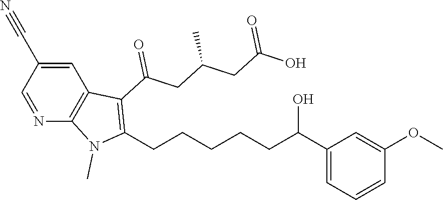 [SMILES: COc1cccc(C(O)CCCCCc2c(C(=O)C[C@H](C)CC(=O)O)c3cc(C#N)cnc3n2C)c1] |
| I-256 | 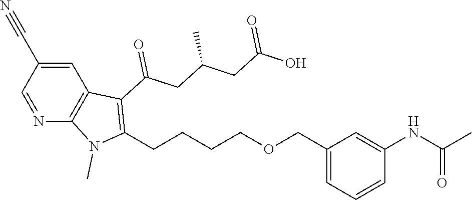 [SMILES: CC(=O)Nc1cccc(COCCCCc2c(C(=O)C[C@H](C)CC(=O)O)c3cc(C#N)cnc3n2C)c1] |
| I-257 | 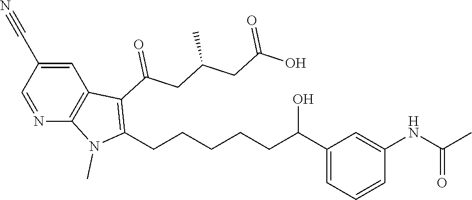 [SMILES: CC(=O)Nc1cccc(C(O)CCCCCc2c(C(=O)C[C@H](C)CC(=O)O)c3cc(C#N)cnc3n2C)c1] |
| I-258 | 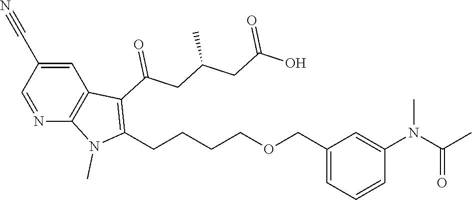 [SMILES: CC(=O)N(C)c1cccc(COCCCCc2c(C(=O)C[C@H](C)CC(=O)O)c3cc(C#N)cnc3n2C)c1] |
| I-259 | 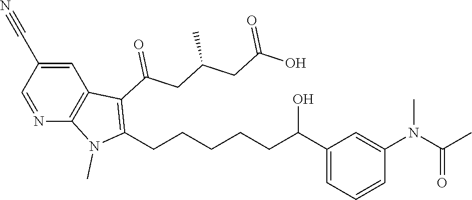 [SMILES: CC(=O)N(C)c1cccc(C(O)CCCCCc2c(C(=O)C[C@H](C)CC(=O)O)c3cc(C#N)cnc3n2C)c1] |
| I-260 | 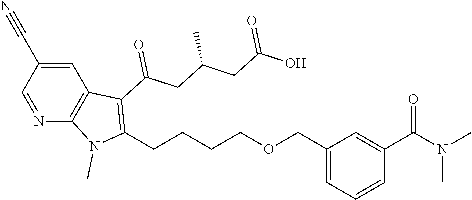 [SMILES: C[C@H](CC(=O)O)CC(=O)c1c(CCCCOCc2cccc(C(=O)N(C)C)c2)n(C)c2ncc(C#N)cc12] |
| I-261 | 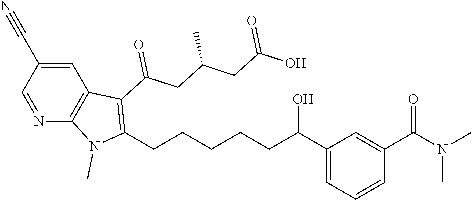 [SMILES: C[C@H](CC(=O)O)CC(=O)c1c(CCCCCC(O)c2cccc(C(=O)N(C)C)c2)n(C)c2ncc(C#N)cc12] |
| I-262 | 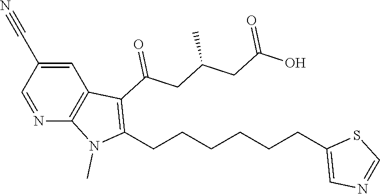 [SMILES: C[C@H](CC(=O)O)CC(=O)c1c(CCCCCCc2cncs2)n(C)c2ncc(C#N)cc12] |
| I-263 | 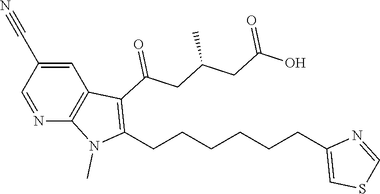 [SMILES: C[C@H](CC(=O)O)CC(=O)c1c(CCCCCCc2cscn2)n(C)c2ncc(C#N)cc12] |
| I-264 | 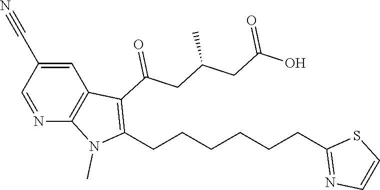 [SMILES: C[C@H](CC(=O)O)CC(=O)c1c(CCCCCCc2nccs2)n(C)c2ncc(C#N)cc12] |
| I-265 | 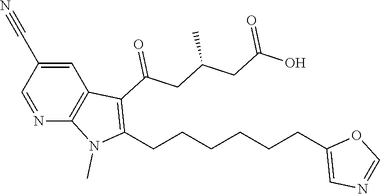 [SMILES: C[C@H](CC(=O)O)CC(=O)c1c(CCCCCCc2cnco2)n(C)c2ncc(C#N)cc12] |
| I-266 | 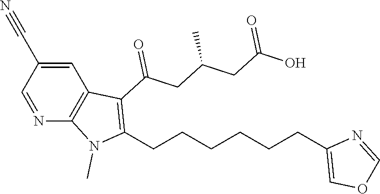 [SMILES: C[C@H](CC(=O)O)CC(=O)c1c(CCCCCCc2cocn2)n(C)c2ncc(C#N)cc12] |
| I-267 | 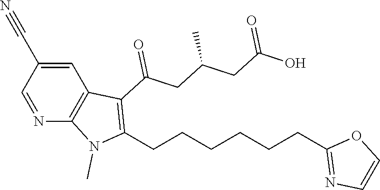 [SMILES: C[C@H](CC(=O)O)CC(=O)c1c(CCCCCCc2ncco2)n(C)c2ncc(C#N)cc12] |
| I-268 | 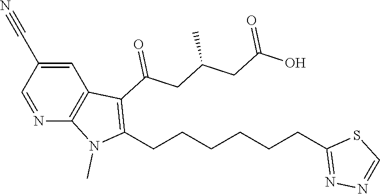 [SMILES: C[C@H](CC(=O)O)CC(=O)c1c(CCCCCCc2nncs2)n(C)c2ncc(C#N)cc12] |
| I-269 | 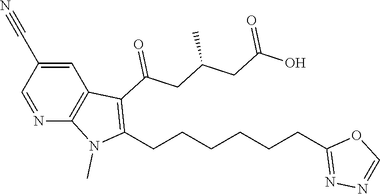 [SMILES: C[C@H](CC(=O)O)CC(=O)c1c(CCCCCCc2nnco2)n(C)c2ncc(C#N)cc12] |
| I-270 | 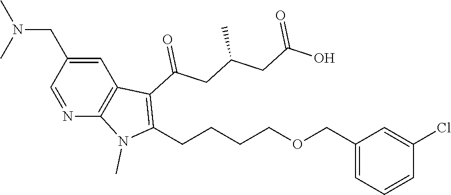 [SMILES: C[C@H](CC(=O)O)CC(=O)c1c(CCCCOCc2cccc(Cl)c2)n(C)c2ncc(CN(C)C)cc12] |
| I-271 | 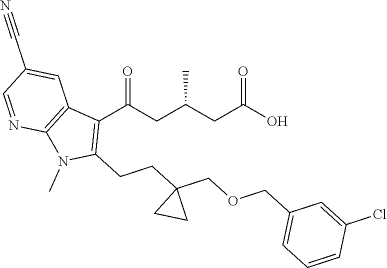 [SMILES: C[C@H](CC(=O)O)CC(=O)c1c(CCC2(COCc3cccc(Cl)c3)CC2)n(C)c2ncc(C#N)cc12] |
| I-272 | 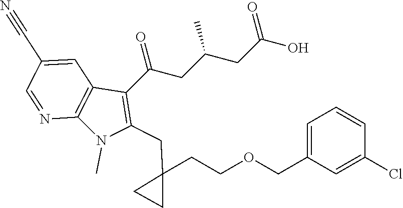 [SMILES: C[C@H](CC(=O)O)CC(=O)c1c(CC2(CCOCc3cccc(Cl)c3)CC2)n(C)c2ncc(C#N)cc12] |
| I-273 | 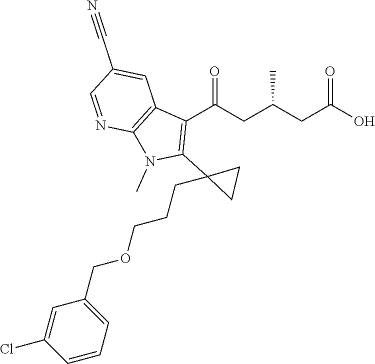 [SMILES: C[C@H](CC(=O)O)CC(=O)c1c(C2(CCCOCc3cccc(Cl)c3)CC2)n(C)c2ncc(C#N)cc12] |
| I-274 | 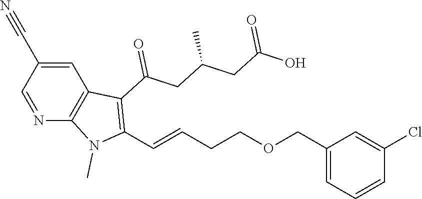 [SMILES: C[C@H](CC(=O)O)CC(=O)c1c(/C=C/CCOCc2cccc(Cl)c2)n(C)c2ncc(C#N)cc12] |
| I-275 | 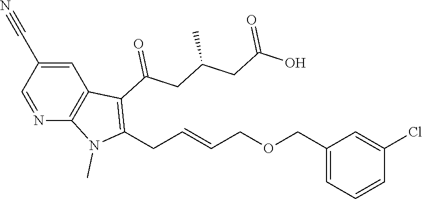 [SMILES: C[C@H](CC(=O)O)CC(=O)c1c(C/C=C/COCc2cccc(Cl)c2)n(C)c2ncc(C#N)cc12] |
| I-276 | 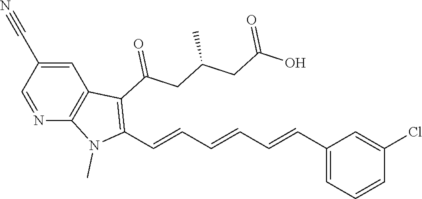 [SMILES: C[C@H](CC(=O)O)CC(=O)c1c(/C=C/C=C/C=C/c2cccc(Cl)c2)n(C)c2ncc(C#N)cc12] |
| I-278 | 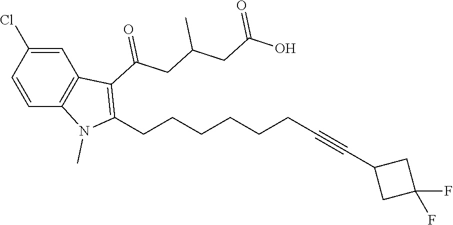 [SMILES: CC(CC(=O)O)CC(=O)c1c(CCCCCCC#CC2CC(F)(F)C2)n(C)c2ccc(Cl)cc12] |
| I-279 | 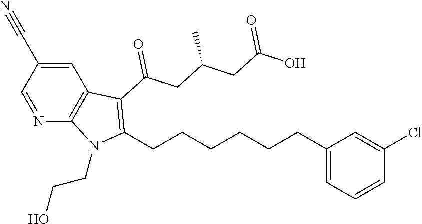 [SMILES: C[C@H](CC(=O)O)CC(=O)c1c(CCCCCCc2cccc(Cl)c2)n(CCO)c2ncc(C#N)cc12] |
| I-280 | 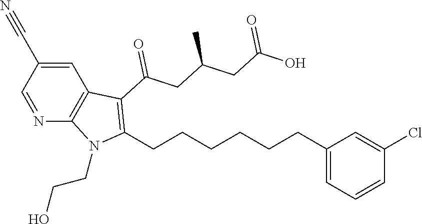 [SMILES: C[C@@H](CC(=O)O)CC(=O)c1c(CCCCCCc2cccc(Cl)c2)n(CCO)c2ncc(C#N)cc12] |
| I-281 | 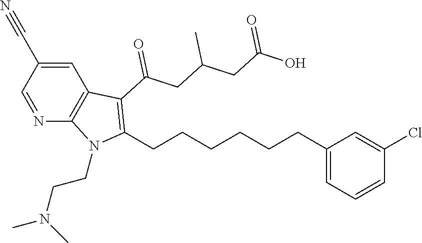 [SMILES: CC(CC(=O)O)CC(=O)c1c(CCCCCCc2cccc(Cl)c2)n(CCN(C)C)c2ncc(C#N)cc12] |
| I-282 | 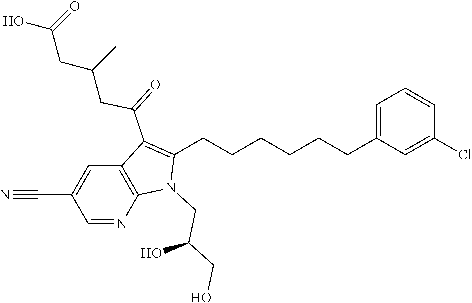 [SMILES: CC(CC(=O)O)CC(=O)c1c(CCCCCCc2cccc(Cl)c2)n(C[C@H](O)CO)c2ncc(C#N)cc12] |
| I-283 | 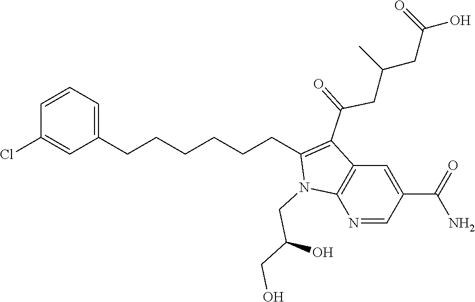 [SMILES: CC(CC(=O)O)CC(=O)c1c(CCCCCCc2cccc(Cl)c2)n(C[C@@H](O)CO)c2ncc(C(N)=O)cc12] |
| I-284 | 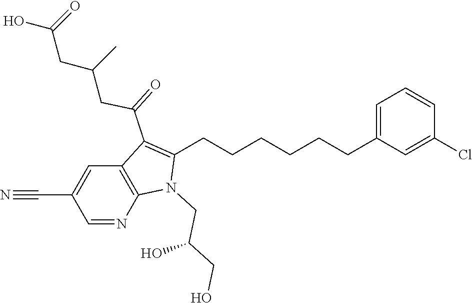 [SMILES: CC(CC(=O)O)CC(=O)c1c(CCCCCCc2cccc(Cl)c2)n(C[C@@H](O)CO)c2ncc(C#N)cc12] |
| I-285 | 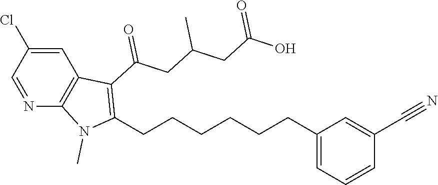 [SMILES: CC(CC(=O)O)CC(=O)c1c(CCCCCCc2cccc(C#N)c2)n(C)c2ncc(Cl)cc12] |
| I-286 | 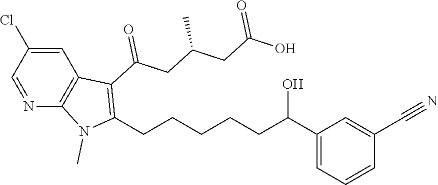 [SMILES: C[C@H](CC(=O)O)CC(=O)c1c(CCCCCC(O)c2cccc(C#N)c2)n(C)c2ncc(Cl)cc12] |
| I-287 | 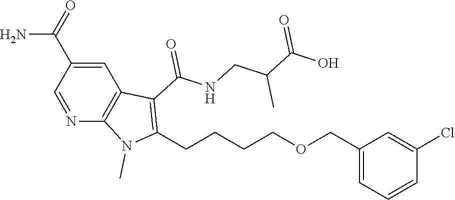 [SMILES: CC(CNC(=O)c1c(CCCCOCc2cccc(Cl)c2)n(C)c2ncc(C(N)=O)cc12)C(=O)O] |
| I-288 | 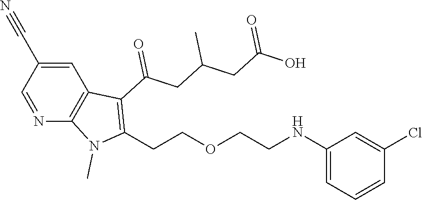 [SMILES: CC(CC(=O)O)CC(=O)c1c(CCOCCNc2cccc(Cl)c2)n(C)c2ncc(C#N)cc12] |
| I-289 | 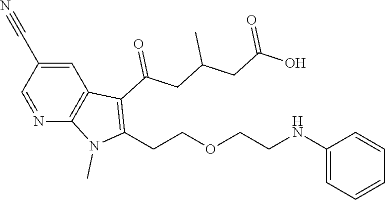 [SMILES: CC(CC(=O)O)CC(=O)c1c(CCOCCNc2ccccc2)n(C)c2ncc(C#N)cc12] |
| I-290 | 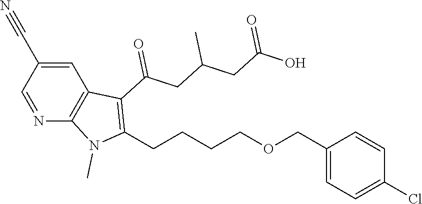 [SMILES: CC(CC(=O)O)CC(=O)c1c(CCCCOCc2ccc(Cl)cc2)n(C)c2ncc(C#N)cc12] |
| I-291 | 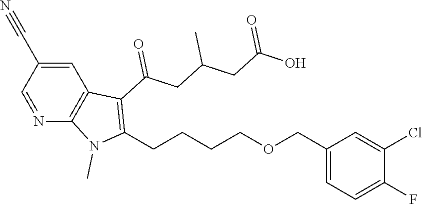 [SMILES: CC(CC(=O)O)CC(=O)c1c(CCCCOCc2ccc(F)c(Cl)c2)n(C)c2ncc(C#N)cc12] |
| I-292 | 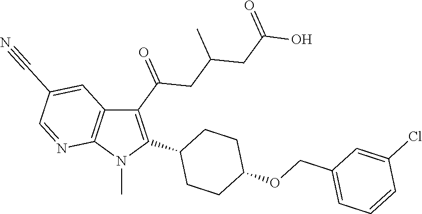 [SMILES: CC(CC(=O)O)CC(=O)c1c2cc(C#N)cnc2n(C)c1[C@H]1CC[C@@H](OCc2cccc(Cl)c2)CC1] |
| I-293 | 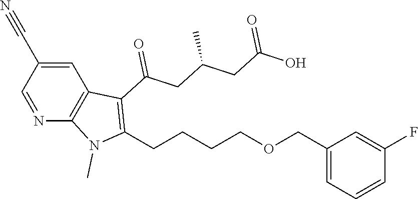 [SMILES: C[C@H](CC(=O)O)CC(=O)c1c(CCCCOCc2cccc(F)c2)n(C)c2ncc(C#N)cc12] |
| I-294 | 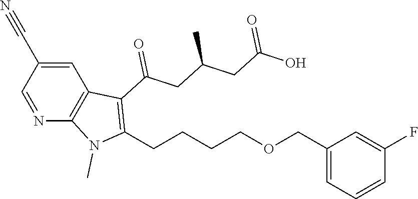 [SMILES: C[C@@H](CC(=O)O)CC(=O)c1c(CCCCOCc2cccc(F)c2)n(C)c2ncc(C#N)cc12] |
| I-295 | 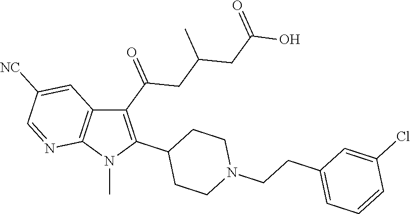 [SMILES: CC(CC(=O)O)CC(=O)c1c(C2CCN(CCc3cccc(Cl)c3)CC2)n(C)c2ncc(C#N)cc12] |
| I-296 | 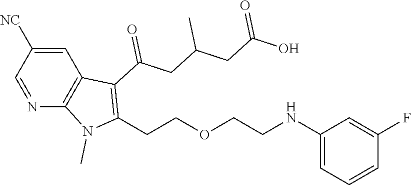 [SMILES: CC(CC(=O)O)CC(=O)c1c(CCOCCNc2cccc(F)c2)n(C)c2ncc(C#N)cc12] |
| I-297 | 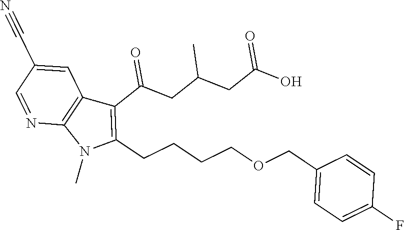 [SMILES: CC(CC(=O)O)CC(=O)c1c(CCCCOCc2ccc(F)cc2)n(C)c2ncc(C#N)cc12] |
| I-298 | 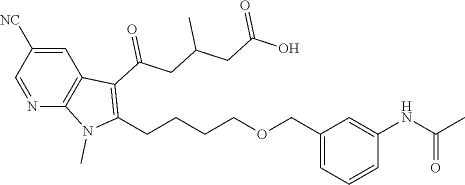 [SMILES: CC(=O)Nc1cccc(COCCCCc2c(C(=O)CC(C)CC(=O)O)c3cc(C#N)cnc3n2C)c1] |
| I-299 | 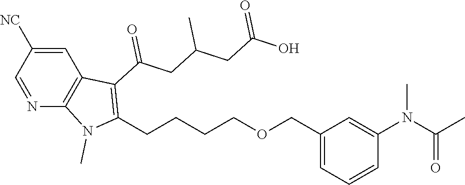 [SMILES: CC(=O)N(C)c1cccc(COCCCCc2c(C(=O)CC(C)CC(=O)O)c3cc(C#N)cnc3n2C)c1] |
| I-300 | 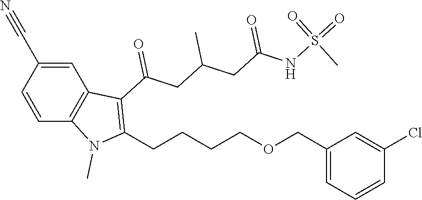 [SMILES: CC(CC(=O)NS(C)(=O)=O)CC(=O)c1c(CCCCOCc2cccc(Cl)c2)n(C)c2ccc(C#N)cc12] |
| I-301 | 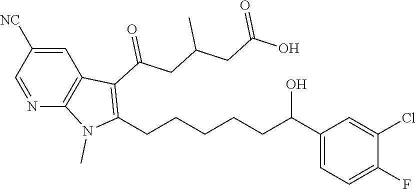 [SMILES: CC(CC(=O)O)CC(=O)c1c(CCCCCC(O)c2ccc(F)c(Cl)c2)n(C)c2ncc(C#N)cc12] |
| I-302 | 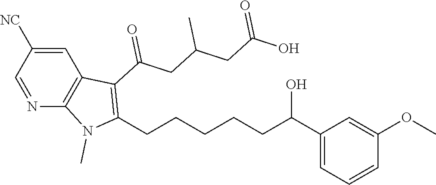 [SMILES: COc1cccc(C(O)CCCCCc2c(C(=O)CC(C)CC(=O)O)c3cc(C#N)cnc3n2C)c1] |
| I-303 | 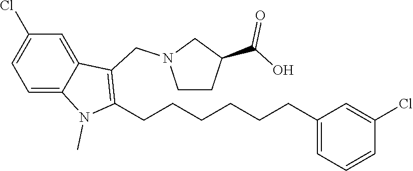 [SMILES: Cn1c(CCCCCCc2cccc(Cl)c2)c(CN2CC[C@H](C(=O)O)C2)c2cc(Cl)ccc21] |
| I-304 | 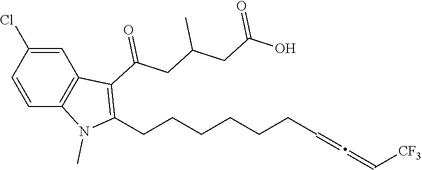 [SMILES: CC(CC(=O)O)CC(=O)c1c(CCCCCCC=C=CC(F)(F)F)n(C)c2ccc(Cl)cc12] |
| I-305 | 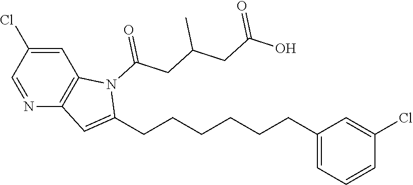 [SMILES: CC(CC(=O)O)CC(=O)n1c(CCCCCCc2cccc(Cl)c2)cc2ncc(Cl)cc21] |
| I-306 | 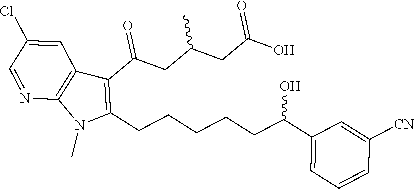 [SMILES: CC(CC(=O)O)CC(=O)c1c(CCCCCC(O)c2cccc(C#N)c2)n(C)c2ncc(Cl)cc12] Single Stereoisomer A |
| I-307 | 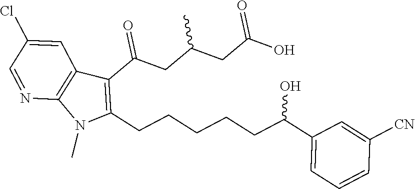 [SMILES: CC(CC(=O)O)CC(=O)c1c(CCCCCC(O)c2cccc(C#N)c2)n(C)c2ncc(Cl)cc12] Single Stereoisomer B |
| I-308 | 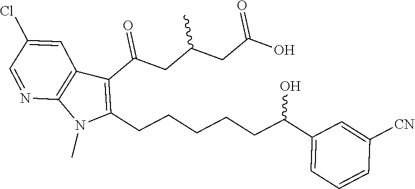 [SMILES: CC(CC(=O)O)CC(=O)c1c(CCCCCC(O)c2cccc(C#N)c2)n(C)c2ncc(Cl)cc12] Single Stereoisomer C |
| I-309 | 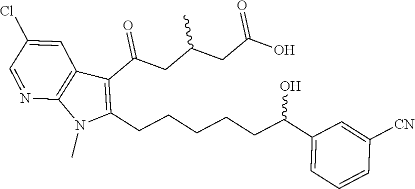 [SMILES: CC(CC(=O)O)CC(=O)c1c(CCCCCC(O)c2cccc(C#N)c2)n(C)c2ncc(Cl)cc12] Single Stereoisomer D |
| I-310 | 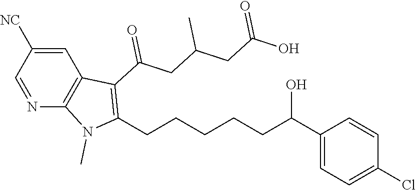 [SMILES: CC(CC(=O)O)CC(=O)c1c(CCCCCC(O)c2ccc(Cl)cc2)n(C)c2ncc(C#N)cc12] |
| I-311 | 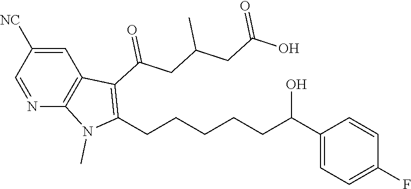 [SMILES: CC(CC(=O)O)CC(=O)c1c(CCCCCC(O)c2ccc(F)cc2)n(C)c2ncc(C#N)cc12] |
| I-312 | 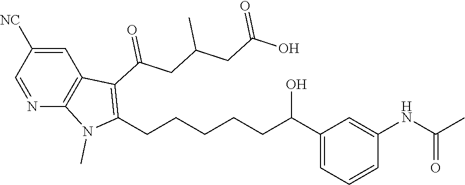 [SMILES: CC(=O)Nc1cccc(C(O)CCCCCc2c(C(=O)CC(C)CC(=O)O)c3cc(C#N)cnc3n2C)c1] |
| I-313 | 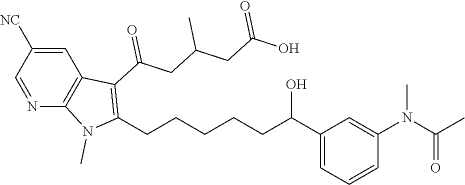 [SMILES: CC(=O)N(C)c1cccc(C(O)CCCCCc2c(C(=O)CC(C)CC(=O)O)c3cc(C#N)cnc3n2C)c1] |
| I-314 | 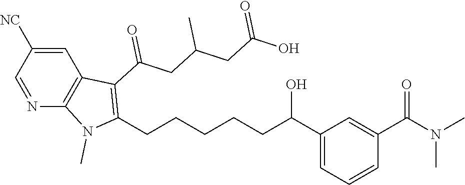 [SMILES: CC(CC(=O)O)CC(=O)c1c(CCCCCC(O)c2cccc(C(=O)N(C)C)c2)n(C)c2ncc(C#N)cc12] |
| I-315 | 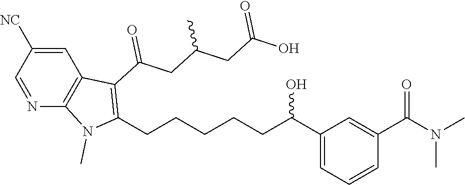 [SMILES: CC(CC(=O)O)CC(=O)c1c(CCCCCC(O)c2cccc(C(=O)N(C)C)c2)n(C)c2ncc(C#N)cc12] Single Stereoisomer A |
| I-316 | 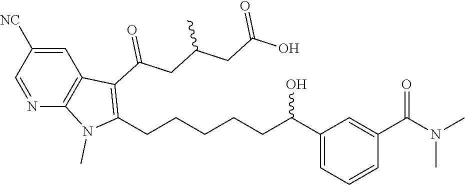 [SMILES: CC(CC(=O)O)CC(=O)c1c(CCCCCC(O)c2cccc(C(=O)N(C)C)c2)n(C)c2ncc(C#N)cc12] Single Stereoisomer B |
| I-317 | 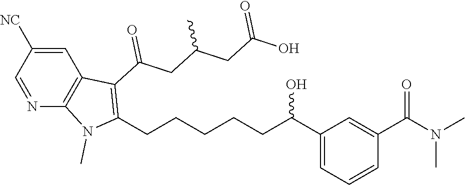 [SMILES: CC(CC(=O)O)CC(=O)c1c(CCCCCC(O)c2cccc(C(=O)N(C)C)c2)n(C)c2ncc(C#N)cc12] Single Stereoisomer C |
| I-318 | 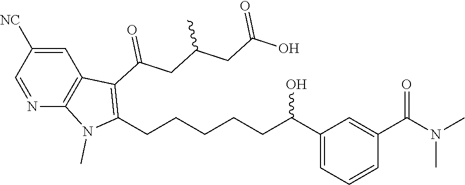 [SMILES: CC(CC(=O)O)CC(=O)c1c(CCCCCC(O)c2cccc(C(=O)N(C)C)c2)n(C)c2ncc(C#N)cc12] Single Stereoisomer D |
| I-319 | 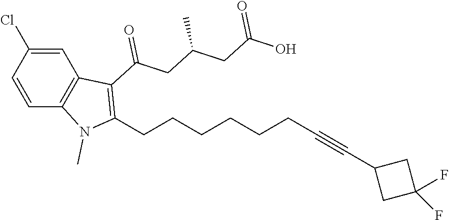 [SMILES: C[C@H](CC(=O)O)CC(=O)c1c(CCCCCCC#CC2CC(F)(F)C2)n(C)c2ccc(Cl)cc12] |
| I-320 | 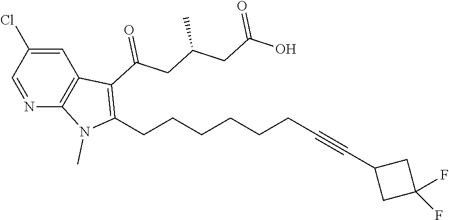 [SMILES: C[C@H](CC(=O)O)CC(=O)c1c(CCCCCCC#CC2CC(F)(F)C2)n(C)c2ncc(Cl)cc12] |
| I-321 | 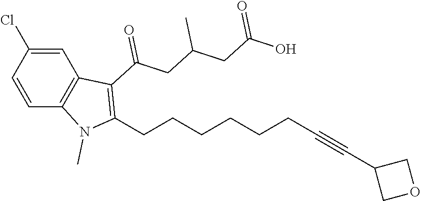 [SMILES: CC(CC(=O)O)CC(=O)c1c(CCCCCCC#CC2COC2)n(C)c2ccc(Cl)cc12] |
| I-322 | 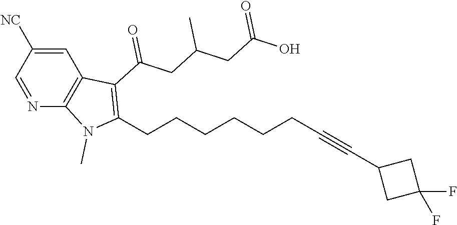 [SMILES: CC(CC(=O)O)CC(=O)c1c(CCCCCCC#CC2CC(F)(F)C2)n(C)c2ncc(C#N)cc12] |
| I-323 | 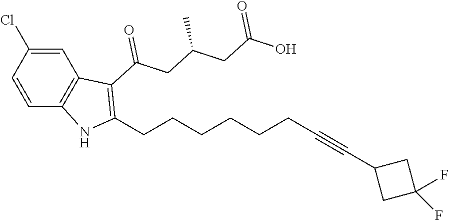 [SMILES: C[C@H](CC(=O)O)CC(=O)c1c(CCCCCCC#CC2CC(F)(F)C2)[nH]c2ccc(Cl)cc12] |
| I-324 | 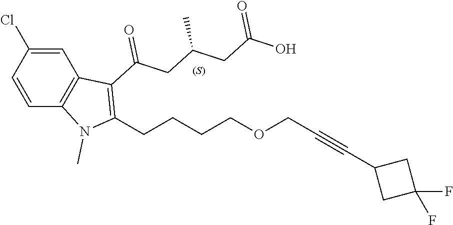 |
| I-325 | 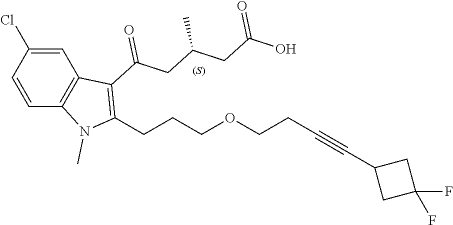 |
| I-326 | 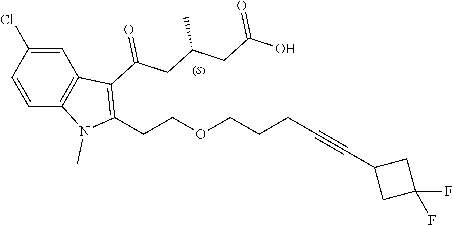 |
| I-327 | 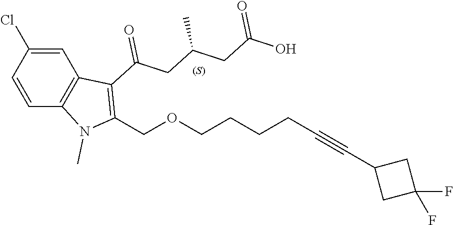 |
| I-328 | 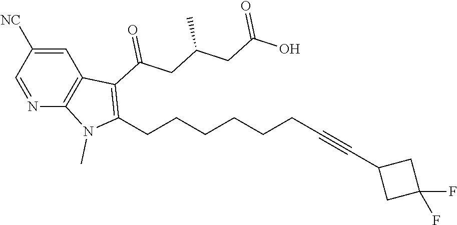 [SMILES: C[C@H](CC(=O)O)CC(=O)c1c(CCCCCCC#CC2CC(F)(F)C2)n(C)c2ncc(C#N)cc12] |
| I-329 | 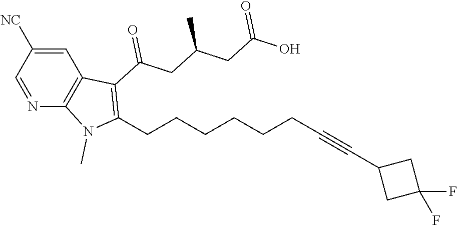 [SMILES: C[C@@H](CC(=O)O)CC(=O)c1c(CCCCCCC#CC2CC(F)(F)C2)n(C)c2ncc(C#N)cc12] |
| I-330 | 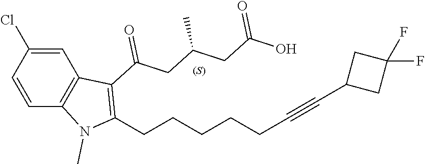 |
| I-331 | 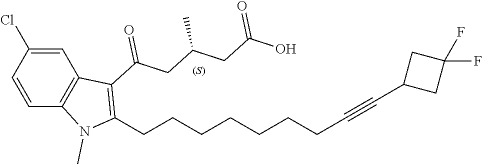 |
| I-332 | 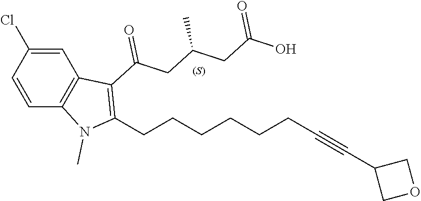 |
| I-333 | 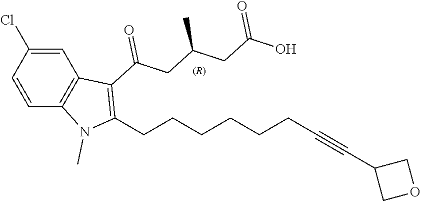 |
| I-334 | 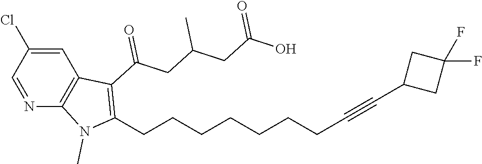 [SMILES: CC(CC(=O)O)CC(=O)c1c(CCCCCCCC#CC2CC(F)(F)C2)n(C)c2ncc(Cl)cc12] |
| I-335 | 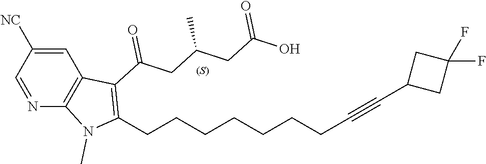 |
| I-336 | 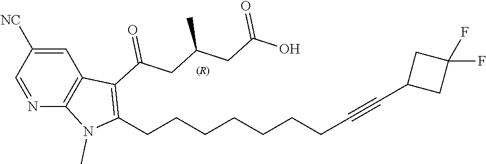 |
| I-337 | 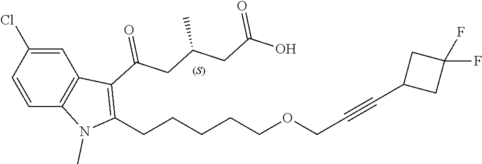 |
| I-338 | 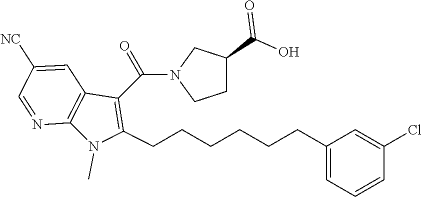 [SMILES: Cn1c(CCCCCCc2cccc(Cl)c2)c(C(=O)N2CC[C@H](C(=O)O)C2)c2cc(C#N)cnc21] |
| I-339 | 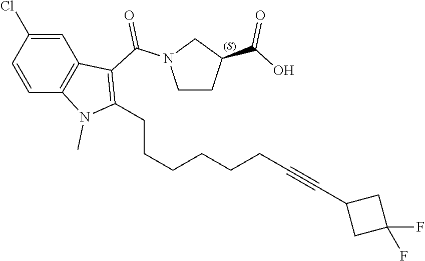 |
| I-340 | 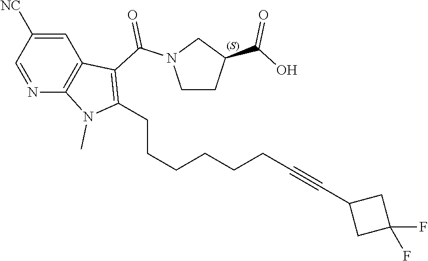 |
| I-341 | 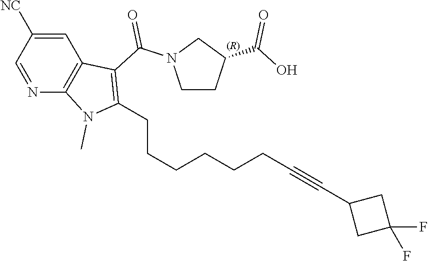 |
| I-342 | 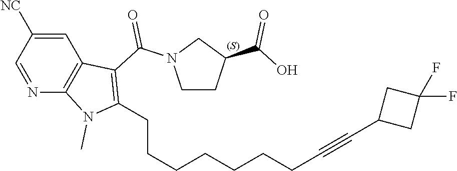 |
| I-343 | 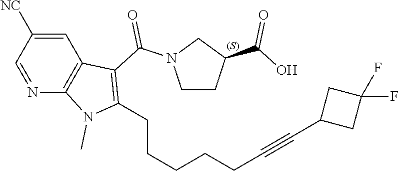 |
| I-344 | 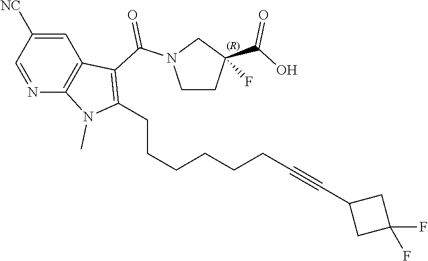 |
| I-345 | 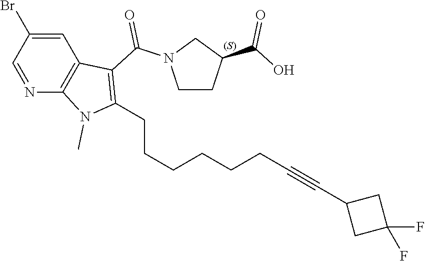 |
| I-346 | 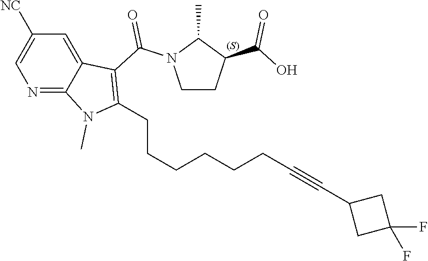 |
| I-347 | 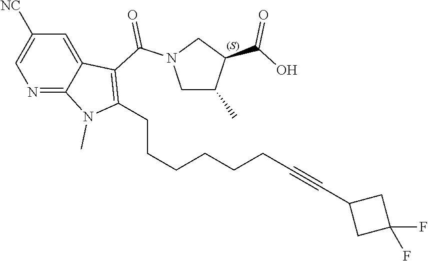 |
| I-348 | 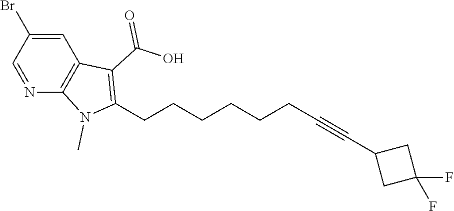 [SMILES: Cn1c(CCCCCCC#CC2CC(F)(F)C2)c(C(=O)O)c2cc(Br)cnc21] |
| I-349 | 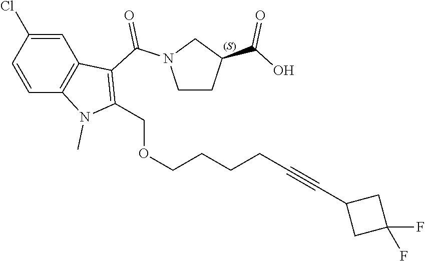 |
| I-350 | 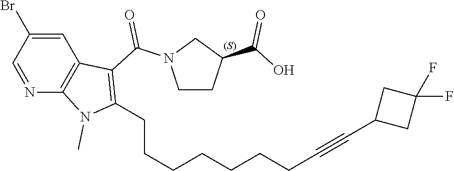 |
| I-351 | 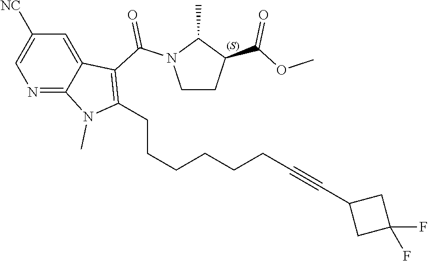 |
| I-352 | 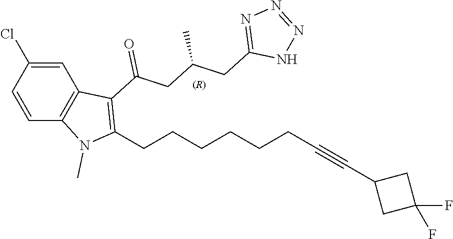 |
| I-353 | 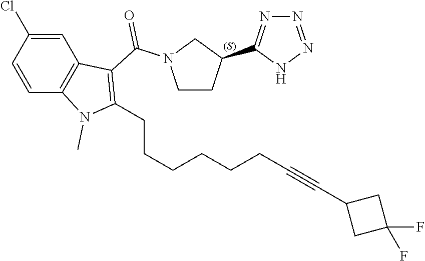 |
| I-354 | 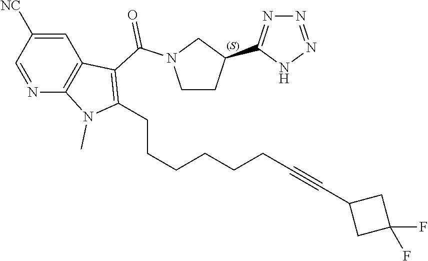 |
| I-355 | 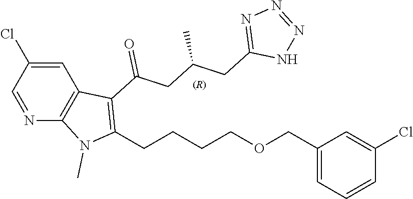 |
| I-356 | 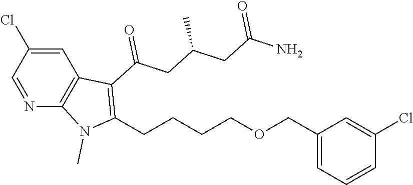 [SMILES: C[C@H](CC(N)=O)CC(=O)c1c(CCCCOCc2cccc(Cl)c2)n(C)c2ncc(Cl)cc12] |
| I-357 | 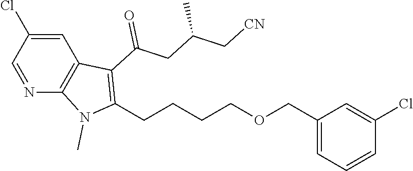 [SMILES: C[C@H](CC#N)CC(=O)c1c(CCCCOCc2cccc(Cl)c2)n(C)c2ncc(Cl)cc12] |
| I-358 | 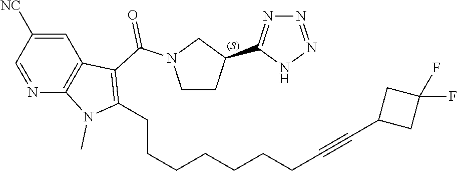 |
| I-359 | 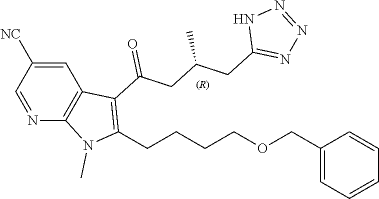 |
| I-360 | 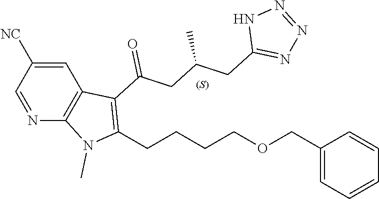 |
| I-361 | 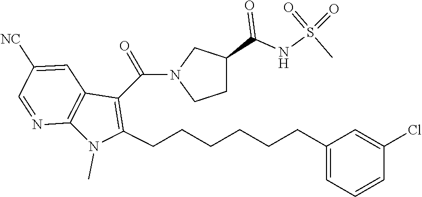 [SMILES: Cn1c(CCCCCCc2cccc(Cl)c2)c(C(=O)N2CC[C@H](C(=O)NS(C)(=O)=O)C2)c2cc(C#N)cnc21] |
| I-362 | 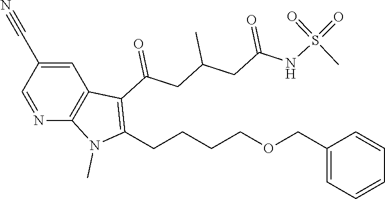 [SMILES: CC(CC(=O)NS(C)(=O)=O)CC(=O)c1c(CCCCOCc2ccccc2)n(C)c2ncc(C#N)cc12] |
| I-363 | 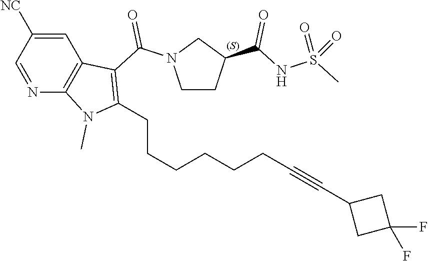 |
| I-364 | 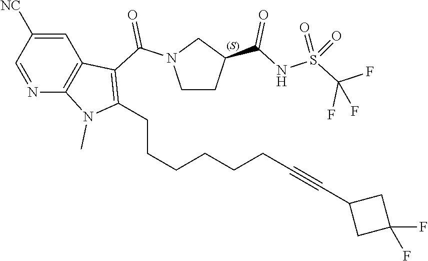 |
| I-366 | 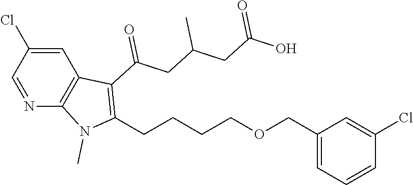 [SMILES: CC(CC(=O)O)CC(=O)c1c(CCCCOCc2cccc(Cl)c2)n(C)c2ncc(Cl)cc12] |
| I-367 | 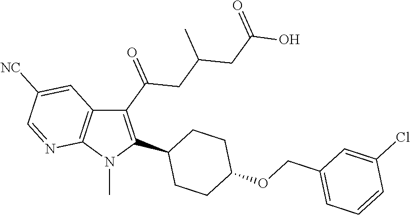 [SMILES: CC(CC(=O)O)CC(=O)c1c2cc(C#N)cnc2n(C)c1[C@H]1CC[C@H](OCc2cccc(Cl)c2)CC1] |
| I-368 | 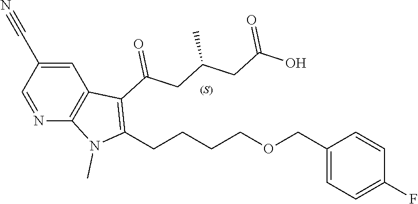 |
| I-369 | 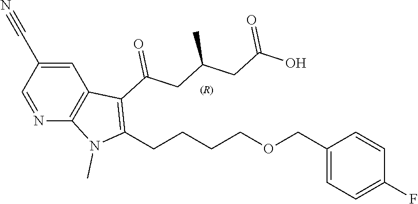 |
| I-370 | 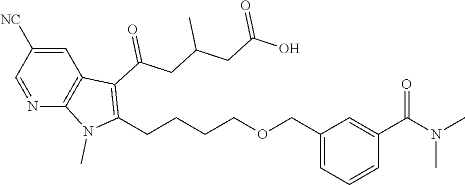 [SMILES: CC(CC(=O)O)CC(=O)c1c(CCCCOCc2cccc(C(=O)N(C)C)c2)n(C)c2ncc(C#N)cc12] |
| I-371 | 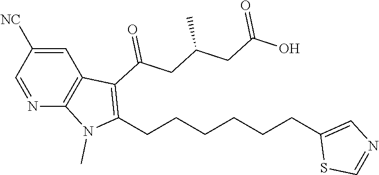 [SMILES: C[C@H](CC(=O)O)CC(=O)c1c(CCCCCCc2cncs2)n(C)c2ncc(C#N)cc12] |
| I-372 | 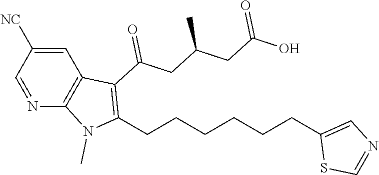 [SMILES: C[C@@H](CC(=O)O)CC(=O)c1c(CCCCCCc2cncs2)n(C)c2ncc(C#N)cc12] |
| I-373 | 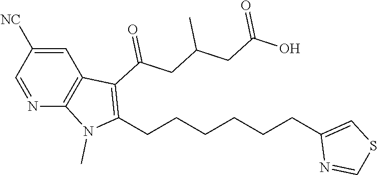 [SMILES: CC(CC(=O)O)CC(=O)c1c(CCCCCCc2cscn2)n(C)c2ncc(C#N)cc12] |
| I-374 | 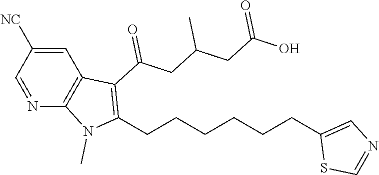 [SMILES: CC(CC(=O)O)CC(=O)c1c(CCCCCCc2cncs2)n(C)c2ncc(C#N)cc12] |
| I-375 | 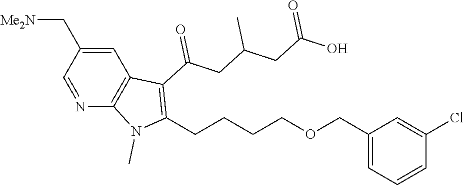 [SMILES: CC(CC(=O)O)CC(=O)c1c(CCCCOCc2cccc(Cl)c2)n(C)c2ncc(CN(C)C)cc12] |
| I-376 | 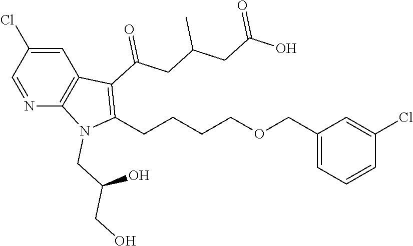 [SMILES: CC(CC(=O)O)CC(=O)c1c(CCCCOCc2cccc(Cl)c2)n(C[C@@H](O)CO)c2ncc(Cl)cc12] |
| I-377 | 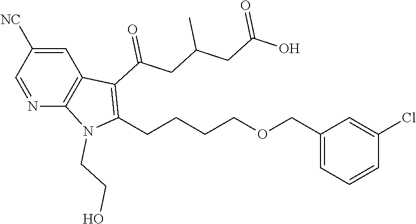 [SMILES: CC(CC(=O)O)CC(=O)c1c(CCCCOCc2cccc(Cl)c2)n(CCO)c2ncc(C#N)cc12] |
| I-378 | 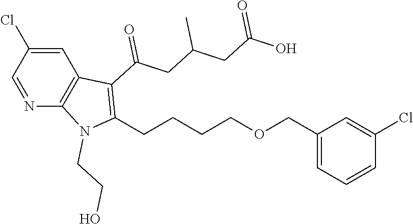 [SMILES: CC(CC(=O)O)CC(=O)c1c(CCCCOCc2cccc(Cl)c2)n(CCO)c2ncc(Cl)cc12] |
| I-379 | 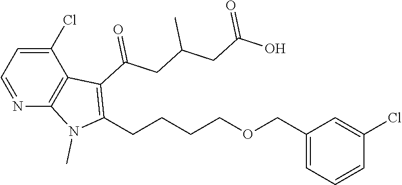 [SMILES: CC(CC(=O)O)CC(=O)c1c(CCCCOCc2cccc(Cl)c2)n(C)c2nccc(Cl)c12] |
| I-380 | 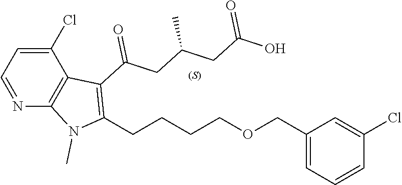 |
| I-381 | 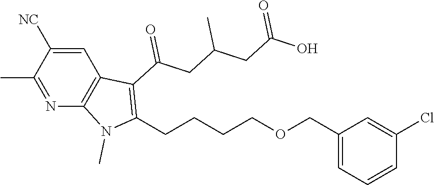 [SMILES: Cc1nc2c(cc1C#N)c(C(=O)CC(C)CC(=O)O)c(CCCCOCc1cccc(Cl)c1)n2C] |
| I-382 | 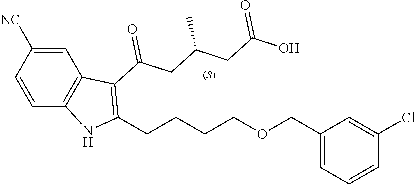 |
| I-383 | 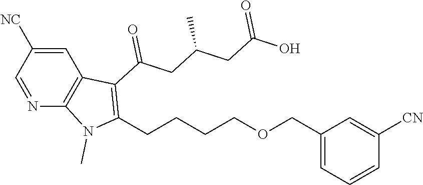 [SMILES: C[C@H](CC(=O)O)CC(=O)c1c(CCCCOCc2cccc(C#N)c2)n(C)c2ncc(C#N)cc12] |
| I-384 | 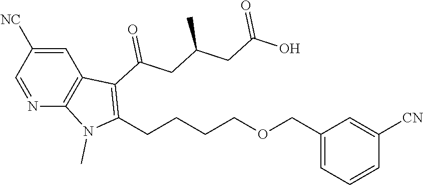 [SMILES: C[C@@H](CC(=O)O)CC(=O)c1c(CCCCOCc2cccc(C#N)c2)n(C)c2ncc(C#N)cc12] |
| I-385 | 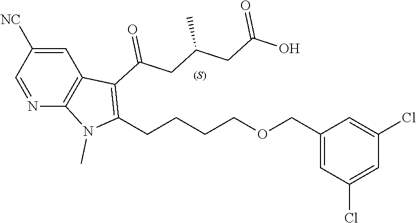 |
| I-386 | 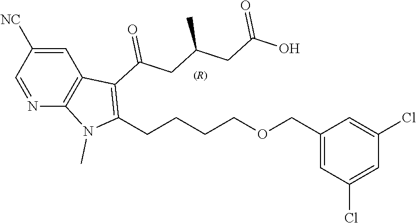 |
| I-387 | 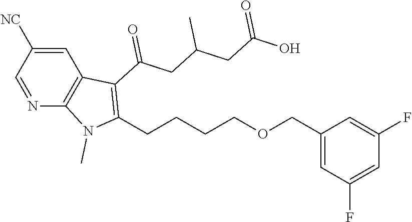 [SMILES: CC(CC(=O)O)CC(=O)c1c(CCCCOCc2cc(F)cc(F)c2)n(C)c2ncc(C#N)cc12] |
| I-388 | 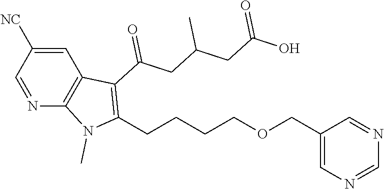 [SMILES: CC(CC(=O)O)CC(=O)c1c(CCCCOCc2cncnc2)n(C)c2ncc(C#N)cc12] |
| I-389 | 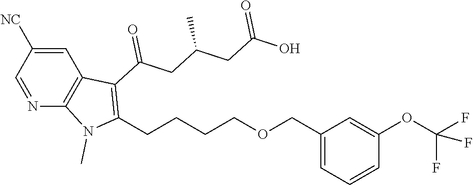 [SMILES: C[C@H](CC(=O)O)CC(=O)c1c(CCCCOCc2cccc(OC(F)(F)F)c2)n(C)c2ncc(C#N)cc12] |
| I-390 | 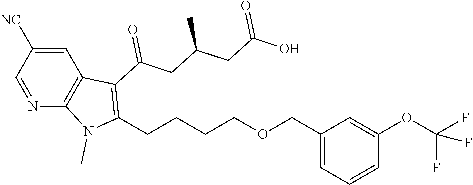 [SMILES: C[C@@H](CC(=O)O)CC(=O)c1c(CCCCOCc2cccc(OC(F)(F)F)c2)n(C)c2ncc(C#N)cc12] |
| I-391 | 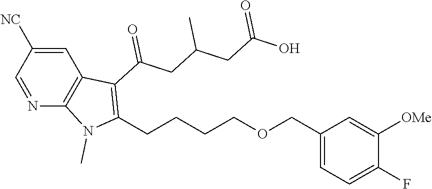 [SMILES: COc1cc(COCCCCc2c(C(=O)CC(C)CC(=O)O)c3cc(C#N)cnc3n2C)ccc1F] |
| I-392 | 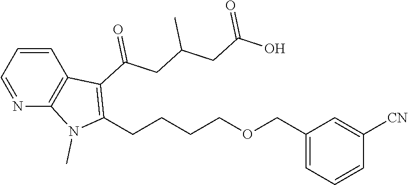 [SMILES: CC(CC(=O)O)CC(=O)c1c(CCCCOCc2cccc(C#N)c2)n(C)c2ncccc12] |
| I-393 | 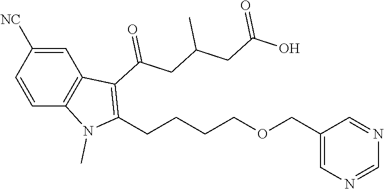 [SMILES: CC(CC(=O)O)CC(=O)c1c(CCCCOCc2cncnc2)n(C)c2ccc(C#N)cc12] |
| I-394 | 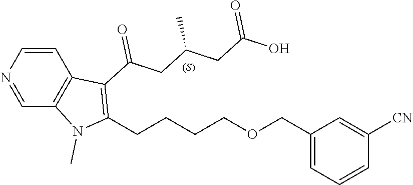 |
| I-395 | 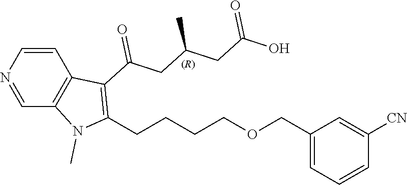 |
| I-396 | 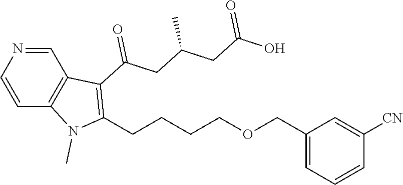 |
| I-397 | 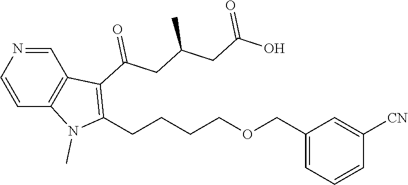 [SMILES: C[C@@H](CC(=O)O)CC(=O)c1c(CCCCOCc2cccc(C#N)c2)n(C)c2ccncc12] |
| I-398 | 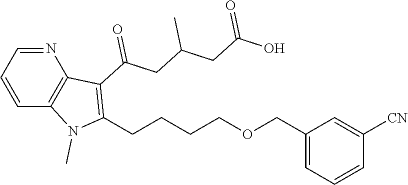 [SMILES: CC(CC(=O)O)CC(=O)c1c(CCCCOCc2cccc(C#N)c2)n(C)c2cccnc12] |
| I-399 | 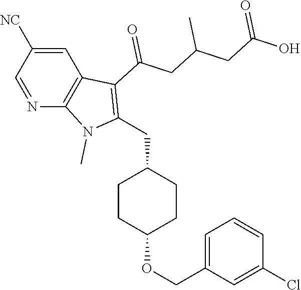 [SMILES: CC(CC(=O)O)CC(=O)c1c(C[C@H]2CC[C@@H](OCc3cccc(Cl)c3)CC2)n(C)c2ncc(C#N)cc12] |
| I-400 | 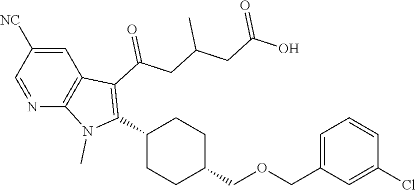 [SMILES: CC(CC(=O)O)CC(=O)c1c2cc(C#N)cnc2n(C)c1[C@H]1CC[C@@H](COCc2cccc(Cl)c2)CC1] |
| I-401 | 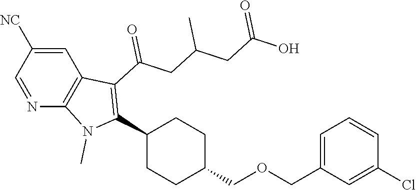 [SMILES: CC(CC(=O)O)CC(=O)c1c2cc(C#N)cnc2n(C)c1[C@H]1CC[C@H](COCc2cccc(Cl)c2)CC1] |
| I-402 | 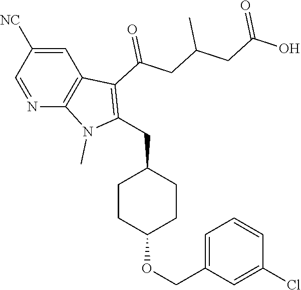 [SMILES: CC(CC(=O)O)CC(=O)c1c(C[C@H]2CC[C@H](OCc3cccc(Cl)c3)CC2)n(C)c2ncc(C#N)cc12] |
| I-403 | 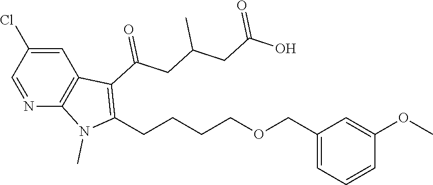 [SMILES: COc1cccc(COCCCCc2c(C(=O)CC(C)CC(=O)O)c3cc(Cl)cnc3n2C)c1] |
| I-404 | 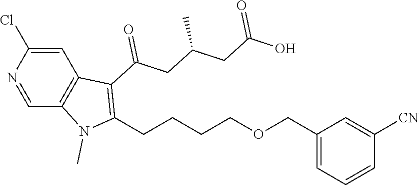 [SMILES: C[C@H](CC(=O)O)CC(=O)c1c(CCCCOCc2cccc(C#N)c2)n(C)c2cnc(Cl)cc12] |
| I-405 | 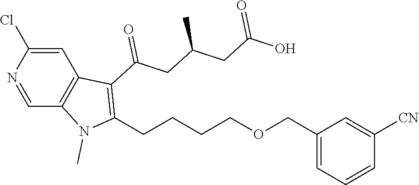 [SMILES: C[C@@H](CC(=O)O)CC(=O)c1c(CCCCOCc2cccc(C#N)c2)n(C)c2cnc(Cl)cc12] |
| I-406 | 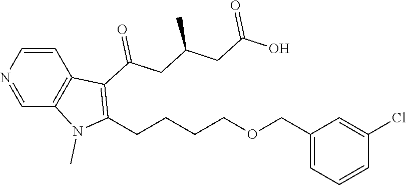 [SMILES: C[C@@H](CC(=O)O)CC(=O)c1c(CCCCOCc2cccc(Cl)c2)n(C)c2cnccc12] |
| I-407 | 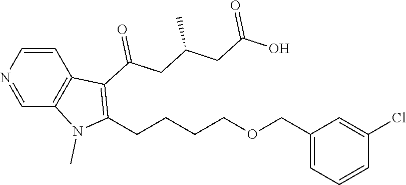 [SMILES: C[C@H](CC(=O)O)CC(=O)c1c(CCCCOCc2cccc(Cl)c2)n(C)c2cnccc12] |
| I-408 | 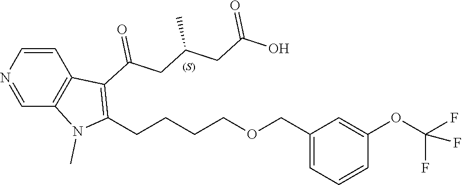 |
| I-409 | 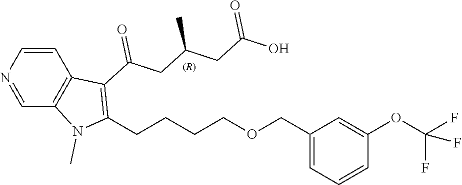 |
| I-410 | 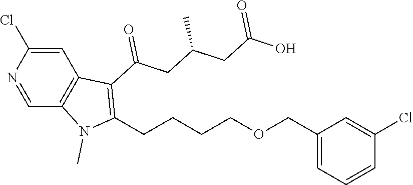 [SMILES: C[C@H](CC(=O)O)CC(=O)c1c(CCCCOCc2cccc(Cl)c2)n(C)c2cnc(Cl)cc12] |
| I-411 | 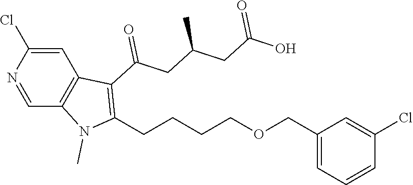 [SMILES: C[C@@H](CC(=O)O)CC(=O)c1c(CCCCOCc2cccc(Cl)c2)n(C)c2cnc(Cl)cc12] |
| I-412 | 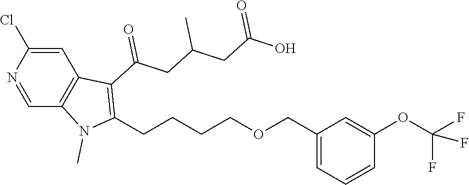 [SMILES: CC(CC(=O)O)CC(=O)c1c(CCCCOCc2cccc(OC(F)(F)F)c2)n(C)c2cnc(Cl)cc12] |
| I-413 | 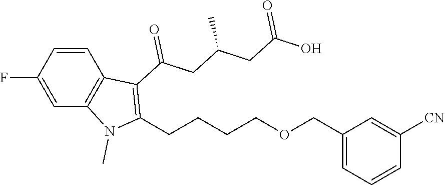 [SMILES: C[C@H](CC(=O)O)CC(=O)c1c(CCCCOCc2cccc(C#N)c2)n(C)c2cc(F)ccc12] |
| I-414 | 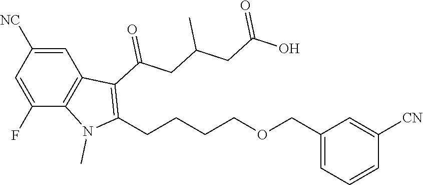 [SMILES: CC(CC(=O)O)CC(=O)c1c(CCCCOCc2cccc(C#N)c2)n(C)c2c(F)cc(C#N)cc12] |
| I-415 | 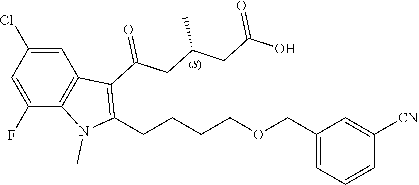 |
| I-416 | 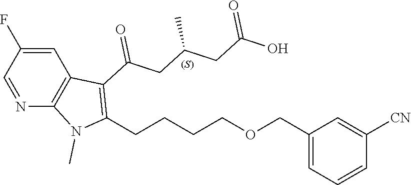 |
| I-417 | 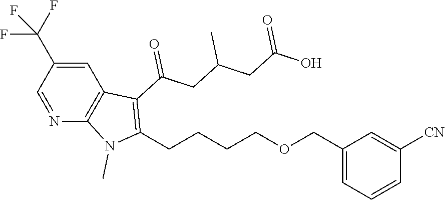 [SMILES: CC(CC(=O)O)CC(=O)c1c(CCCCOCc2cccc(C#N)c2)n(C)c2ncc(C(F)(F)F)cc12] |
| I-418 | 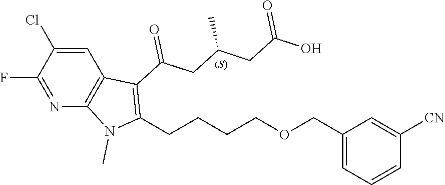 |
| I-419 | 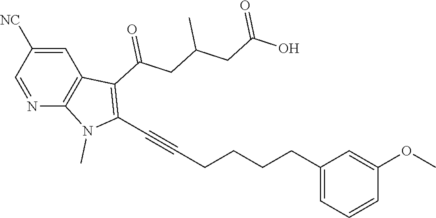 [SMILES: COc1cccc(CCCCC#Cc2c(C(=O)CC(C)CC(=O)O)c3cc(C#N)cnc3n2C)c1] |
| I-420 | 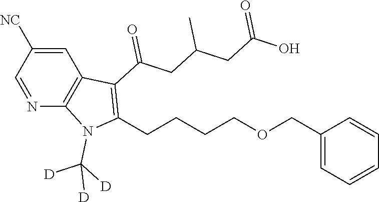 [SMILES: [2H]C([2H])([2H])n1c(CCCCOCc2ccccc2)c(C(=O)CC(C)CC(=O)O)c2cc(C#N)cnc21] |
| I-421 | 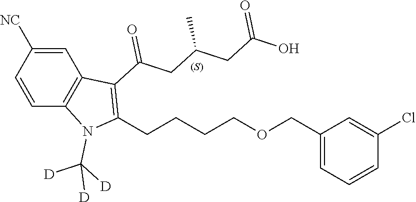 |
| I-422 | 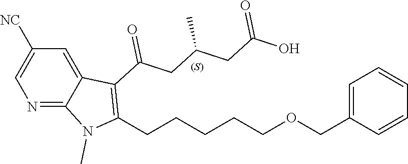 |
| I-423 | 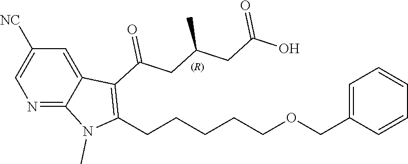 |
| I-424 | 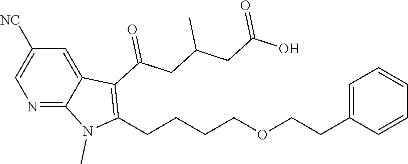 [SMILES: CC(CC(=O)O)CC(=O)c1c(CCCCOCCc2ccccc2)n(C)c2ncc(C#N)cc12] |
| I-425 | 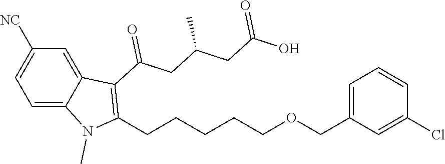 [SMILES: C[C@H](CC(=O)O)CC(=O)c1c(CCCCCOCc2cccc(Cl)c2)n(C)c2ccc(C#N)cc12] |
| I-426 | 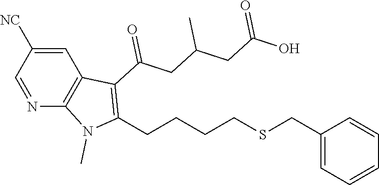 [SMILES: CC(CC(=O)O)CC(=O)c1c(CCCCSCc2ccccc2)n(C)c2ncc(C#N)cc12] |
| I-427 | 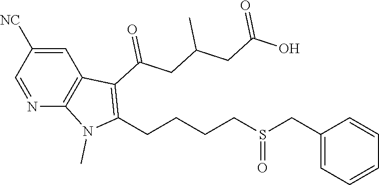 [SMILES: CC(CC(=O)O)CC(=O)c1c(CCCCS(=O)Cc2ccccc2)n(C)c2ncc(C#N)cc12] |
| I-428 | 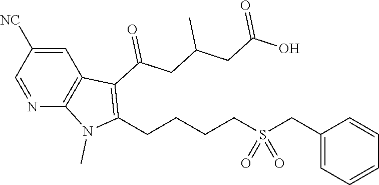 [SMILES: CC(CC(=O)O)CC(=O)c1c(CCCCS(=O)(=O)Cc2ccccc2)n(C)c2ncc(C#N)cc12] |
| I-429 | 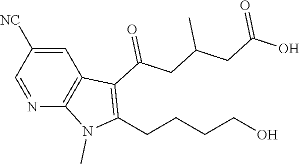 [SMILES: CC(CC(=O)O)CC(=O)c1c(CCCCO)n(C)c2ncc(C#N)cc12] |
| I-430 | 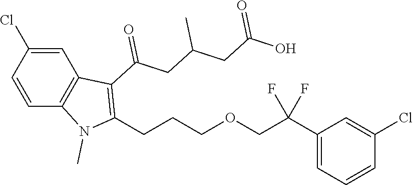 [SMILES: CC(CC(=O)O)CC(=O)c1c(CCCOCC(F)(F)c2cccc(Cl)c2)n(C)c2ccc(Cl)cc12] |
| I-431 | 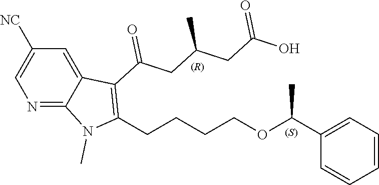 |
| I-432 | 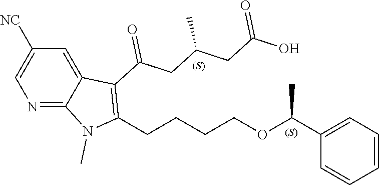 |
| I-433 | 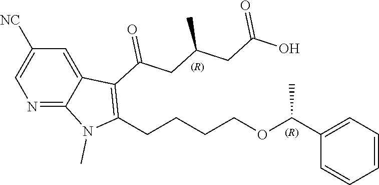 |
| I-434 | 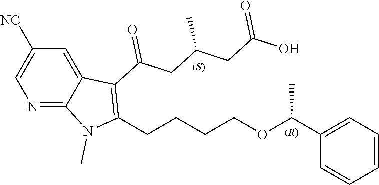 |
| I-435 | 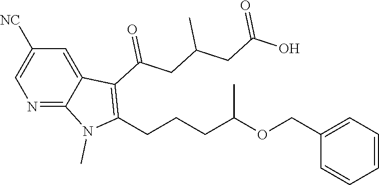 [SMILES: CC(CC(=O)O)CC(=O)c1c(CCCC(C)OCc2ccccc2)n(C)c2ncc(C#N)cc12] |
| I-436 | 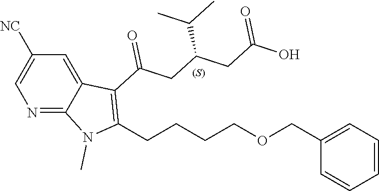 |
| I-437 | 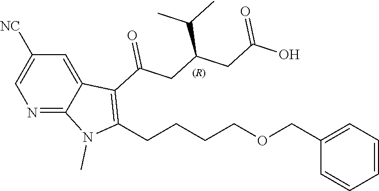 |
| I-438 | 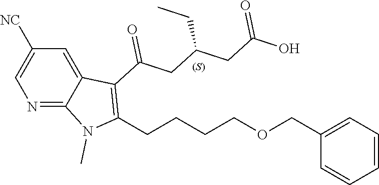 |
| I-439 | 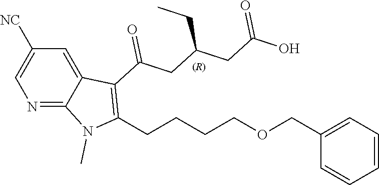 |
| I-440 | 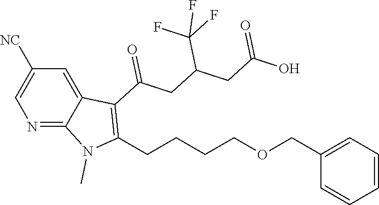 [SMILES: Cn1c(CCCCOCc2ccccc2)c(C(=O)CC(CC(=O)O)C(F)(F)F)c2cc(C#N)cnc21] |
| I-441 | 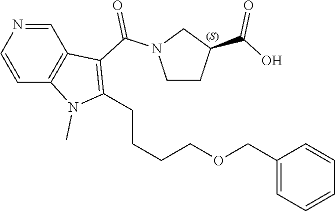 |
| I-442 | 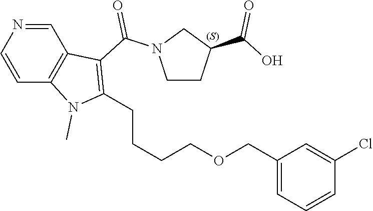 |
| I-443 | 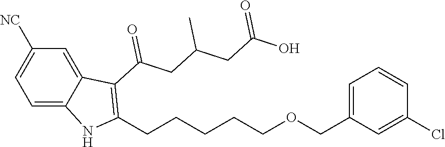 [SMILES: CC(CC(=O)O)CC(=O)c1c(CCCCCOCc2cccc(Cl)c2)[nH]c2ccc(C#N)cc12] |
| I-444 | 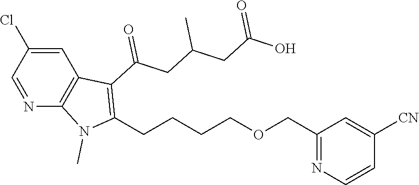 [SMILES: CC(CC(=O)O)CC(=O)c1c(CCCCOCc2cc(C#N)ccn2)n(C)c2ncc(Cl)cc12] |
| I-445 | 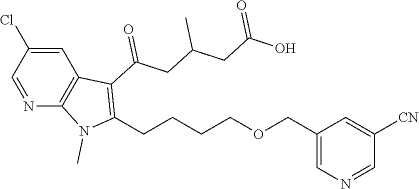 [SMILES: CC(CC(=O)O)CC(=O)c1c(CCCCOCc2cncc(C#N)c2)n(C)c2ncc(Cl)cc12] |
| I-446 | 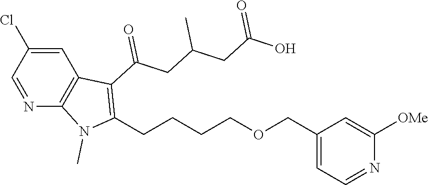 [SMILES: COc1cc(COCCCCc2c(C(=O)CC(C)CC(=O)O)c3cc(Cl)cnc3n2C)ccn1] |
| I-447 | 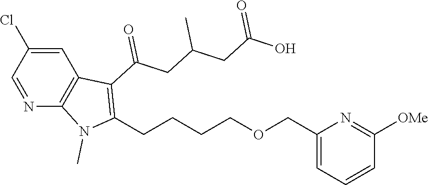 [SMILES: COc1cccc(COCCCCc2c(C(=O)CC(C)CC(=O)O)c3cc(Cl)cnc3n2C)n1] |
| I-448 | 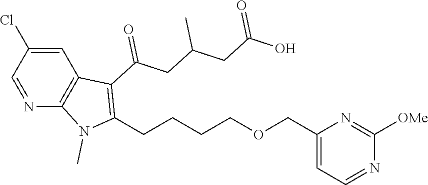 [SMILES: COc1nccc(COCCCCc2c(C(=O)CC(C)CC(=O)O)c3cc(Cl)cnc3n2C)n1] |
| I-449 | 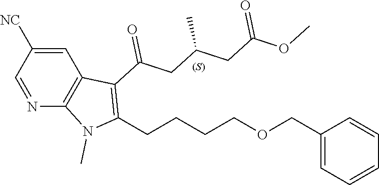 |
| I-450 | 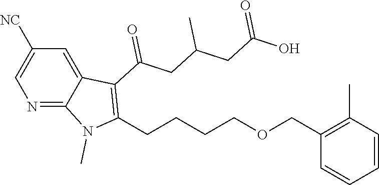 [SMILES: Cc1ccccc1COCCCCc1c(C(=O)CC(C)CC(=O)O)c2cc(C#N)cnc2n1C] |
| I-451 | 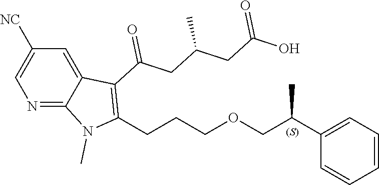 |
| I-452 | 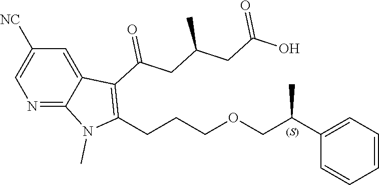 |
| I-453 | 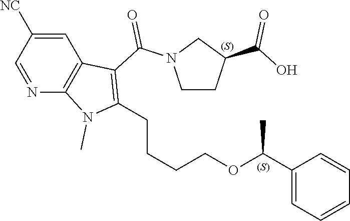 |
| I-454 | 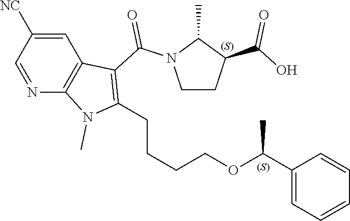 |
| I-455 | 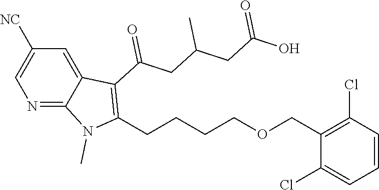 [SMILES: CC(CC(=O)O)CC(=O)c1c(CCCCOCc2c(Cl)cccc2Cl)n(C)c2ncc(C#N)cc12] |
| I-456 | 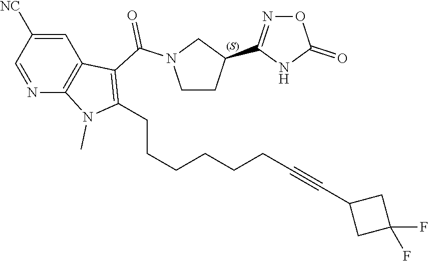 |
| I-457 | 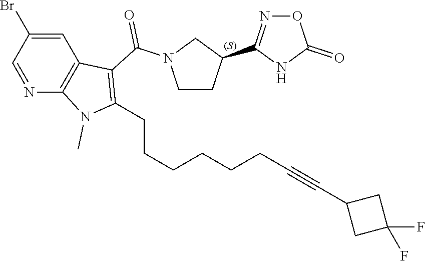 |
| I-458 | 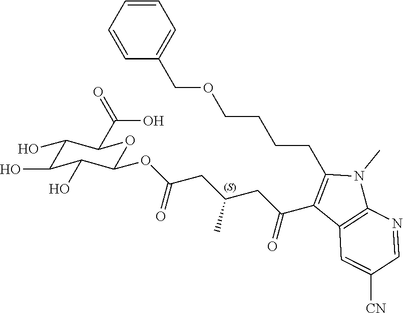 |
| I-459 | 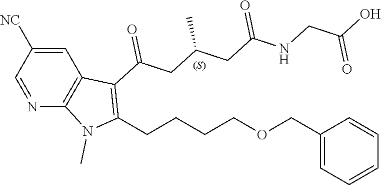 |
| I-460 | 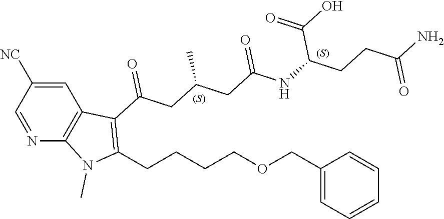 |
| I-461 | 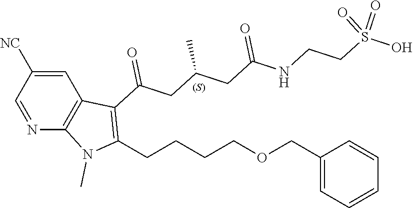 |
| I-462 | 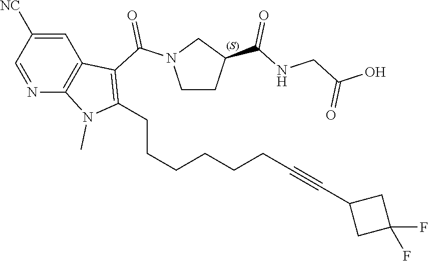 |
| I-463 | 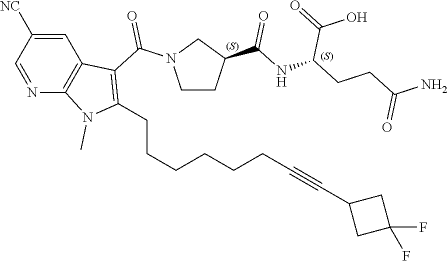 |
| I-464 | 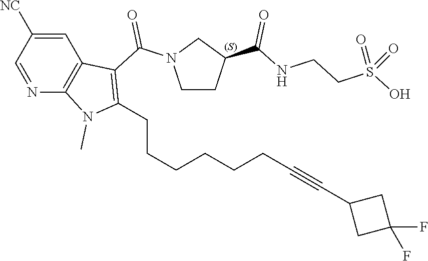 |
| I-465 | 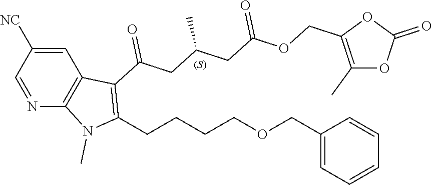 |
| I-466 | 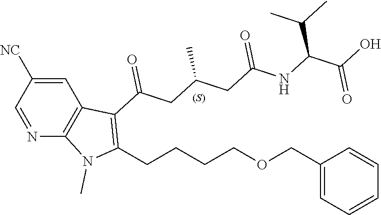 |
| I-467 | 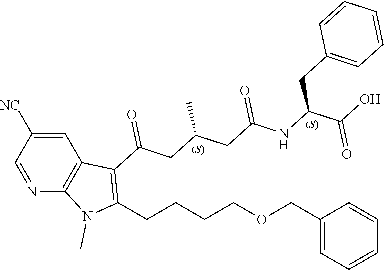 |
| I-468 | 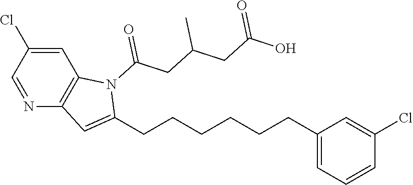 [SMILES: CC(CC(=O)O)CC(=O)n1c(CCCCCCc2cccc(Cl)c2)cc2ncc(Cl)cc21] |
| I-469 | 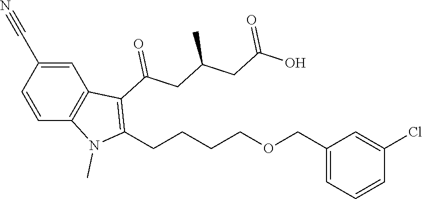 [SMILES: C[C@@H](CC(=O)O)CC(=O)c1c(CCCCOCc2cccc(Cl)c2)n(C)c2ccc(C#N)cc12] |

In some embodiments, the present invention provides a compound set forth in Table 1, above, or an N-oxide, or a pharmaceutically acceptable salt thereof. In some embodiments, the present invention provides a compound set forth in Table 1, above, or a pharmaceutically acceptable salt thereof. In some embodiments, the present invention provides a compound set forth in Table 1, above. In some embodiments, the present invention provides a pharmaceutical composition comprising a compound set forth in Table 1 above, or an N-oxide, or a pharmaceutically acceptable salt thereof, together with a pharmaceutically acceptable carrier, excipient, or diluent.

In some embodiments, the present invention provides a compound selected from Compounds I-13 through I-304, set forth in Table 1, above. In some embodiments, the present invention provides a compound selected from Compounds I-305 through I-467, set forth in Table 1, above. In some embodiments, the present invention provides a compound selected from Compounds I-13 through I-304, set forth in Table 1, above, or an N-oxide, or a pharmaceutically acceptable salt thereof. In some embodiments, the present invention provides a compound selected from Compounds I-305 through I-467, set forth in Table 1, above, or an N-oxide, or a pharmaceutically acceptable salt thereof. In some embodiments, the present invention provides a compound selected from Compounds I-13 through I-304, set forth in Table 1, above, or a pharmaceutically acceptable salt thereof. In some embodiments, the present invention provides a compound selected from Compounds I-305 through I-467, set forth in Table 1, above, or a pharmaceutically acceptable salt thereof.

In some embodiments, the present invention provides a pharmaceutical composition comprising a compound selected from Compounds I-13 through I-304, set forth in Table 1 above, or an N-oxide, or a pharmaceutically acceptable salt thereof, together with a pharmaceutically acceptable carrier, excipient, or diluent. In some embodiments, the present invention provides a pharmaceutical composition comprising a compound selected from Compounds I-305 through I-467, set forth in Table 1 above, or an N-oxide, or a pharmaceutically acceptable salt thereof, together with a pharmaceutically acceptable carrier, excipient, or diluent.

In some embodiments, the present invention provides a compound of formula I as defined above, or an N-oxide, or a pharmaceutically acceptable salt thereof, or a pharmaceutical composition comprising a compound of formula I as defined above, or an N-oxide, or a pharmaceutically acceptable salt thereof, and a pharmaceutically acceptable carrier, adjuvant, or vehicle for use as a medicament.

In chemical structures in Table 1, above, and the Examples, below, stereogenic centers are described according to the Enhanced Stereo Representation format (MDL/Biovia, e.g. using labels “or1”, “or2”, “abs”, “and1”).

Additional exemplary compounds include those set forth in the following table. In certain embodiments, the compound may be a compound from the following table or an N-oxide, or a pharmaceutically acceptable salt thereof.

| Compound No. | Structure |
|---|---|
| I-1 | 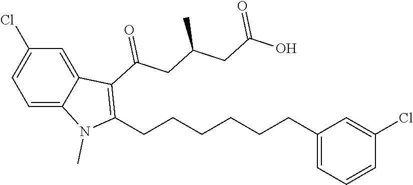 [SMILES: C[C@@H](CC(=O)O)CC(=O)c1c(CCCCCCc2cccc(Cl)c2)n(C)c2ccc(Cl)cc12] |
| I-2 | 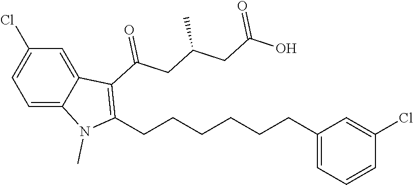 [SMILES: C[C@H](CC(=O)O)CC(=O)c1c(CCCCCCc2cccc(Cl)c2)n(C)c2ccc(Cl)cc12] |
| I-3 | 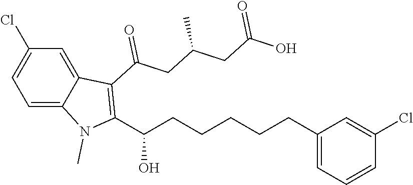 [SMILES: C[C@H](CC(=O)O)CC(=O)c1c([C@@H](O)CCCCCc2cccc(Cl)c2)n(C)c2ccc(Cl)cc12] |
| I-4 | 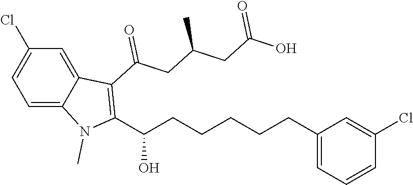 [SMILES: C[C@@H](CC(=O)O)CC(=O)c1c([C@@H](O)CCCCCc2cccc(Cl)c2)n(C)c2ccc(Cl)cc12] |
| I-5 | 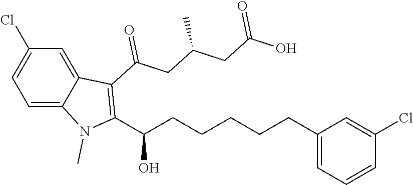 [SMILES: C[C@H](CC(=O)O)CC(=O)c1c([C@H](O)CCCCCc2cccc(Cl)c2)n(C)c2ccc(Cl)cc12] |
| I-6 | 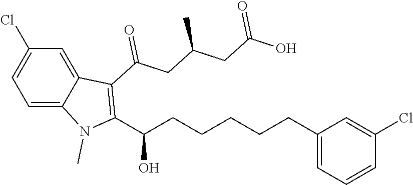 [SMILES: C[C@@H](CC(=O)O)CC(=O)c1c([C@H](O)CCCCCc2cccc(Cl)c2)n(C)c2ccc(Cl)cc12] |
| I-7 | 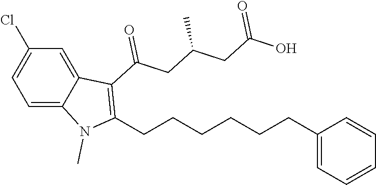 [SMILES: C[C@H](CC(=O)O)CC(=O)c1c(CCCCCCc2ccccc2)n(C)c2ccc(Cl)cc12] |
| I-8 | 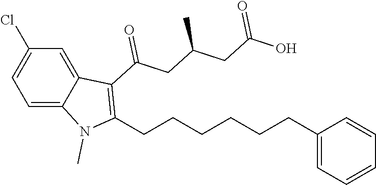 [SMILES: C[C@@H](CC(=O)O)CC(=O)c1c(CCCCCCc2ccccc2)n(C)c2ccc(Cl)cc12] |
| I-9 | 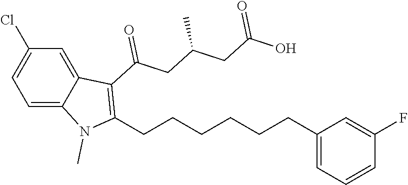 [SMILES: C[C@H](CC(=O)O)CC(=O)c1c(CCCCCCc2cccc(F)c2)n(C)c2ccc(Cl)cc12] |
| I-10 | 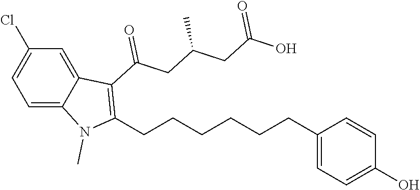 [SMILES: C[C@H](CC(=O)O)CC(=O)c1c(CCCCCCc2ccc(O)cc2)n(C)c2ccc(Cl)cc12] |
| I-11 | 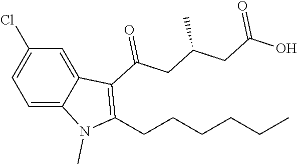 [SMILES: CCCCCCc1c(C(=O)C[C@H](C)CC(=O)O)c2cc(Cl)ccc2n1C] |
| I-12 | 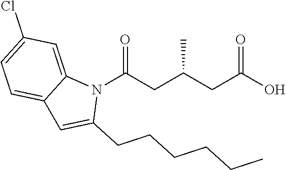 [SMILES: CCCCCCc1cc2ccc(Cl)cc2n1C(=O)C[C@H](C)CC(=O)O] |

In some embodiments, the present invention provides a pharmaceutical composition comprising a compound set forth in the table above, or an N-oxide, or a pharmaceutically acceptable salt thereof, together with a pharmaceutically acceptable carrier, excipient, or diluent.

In some embodiments, the present invention provides a compound in the table above or an N-oxide, or a pharmaceutically acceptable salt thereof, or a pharmaceutical composition comprising a compound in the table above, or an N-oxide, or a pharmaceutically acceptable salt thereof, and a pharmaceutically acceptable carrier, adjuvant, or vehicle for use as a medicament.

In some embodiments, the invention also provides compounds of formula I described herein or pharmaceutical compositions described herein for use in a method for inhibiting OXER1 as described herein, in a method for modulating an immune response in a subject in need thereof as described herein and/or in a method for treating a OXER1-dependent disorder as described herein.

In some embodiments, the invention also provides compounds of formula I described herein or pharmaceutical compositions described herein for use in a method for inhibiting OXER1 as described herein.

In some embodiments, the invention also provides compounds of formula I described herein or pharmaceutical compositions described herein for use in a method for modulating an immune response in a subject in need thereof as described herein.

In some embodiments, the invention also provides compounds of formula I described herein or pharmaceutical compositions described herein for use in a method for treating a OXER1-dependent disorder as described herein.

In some embodiments, the invention also provides the use of a compound of formula I described herein or a pharmaceutical composition described herein for the manufacture of a medicament for inhibiting OXER1, a medicament for modulating an immune response in a subject in need thereof and/or a medicament for treating a OXER1-dependent disorder.

In some embodiments, the invention also provides the use of a compound of formula I described herein or a pharmaceutical composition described herein for the manufacture of a medicament for inhibiting OXER1.

In some embodiments, the invention also provides the use of a compound of formula I described herein or a pharmaceutical composition described herein for the manufacture of a medicament for modulating an immune response in a subject in need thereof.

In some embodiments, the invention also provides the use of a compound of formula I described herein, or a pharmaceutical composition described herein for the manufacture of a medicament treating a OXER1-dependent disorder.

In some embodiments, the invention also provides the use of compounds of formula I described herein or pharmaceutical compositions described herein in a method for inhibiting OXER1 as described herein, in a method for modulating an immune response in a subject in need thereof as described herein and/or in a method for treating a OXER1-dependent disorder as described herein.

In some embodiments, the invention also provides the use of compounds of formula I described herein, or pharmaceutical compositions described herein in a method for inhibiting OXER1 as described herein.

In some embodiments, the invention also provides the use of compounds of formula I described herein, or pharmaceutical compositions described herein in a method for modulating an immune response in a subject in need thereof as described herein.

In some embodiments, the invention also provides the use of compounds of formula I described herein, or pharmaceutical compositions described herein in a method for treating a OXER1-dependent disorder as described herein.

### General Methods of Providing the Present Compounds

The compounds of this invention may be prepared or isolated in general by synthetic and/or semi-synthetic methods known to those skilled in the art for analogous compounds and by methods described in detail in the Examples, herein.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit OXER1, or a mutant thereof, in a biological sample or in a patient. In certain embodiments, the amount of compound in compositions of this invention is such that is effective to measurably inhibit OXER1, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of OXER1, or a mutant thereof.

The subject matter disclosed herein includes prodrugs, metabolites, derivatives, and pharmaceutically acceptable salts of compounds of the invention. Metabolites include compounds produced by a process comprising contacting a compound of the invention with a mammal for a period of time sufficient to yield a metabolic product thereof. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A compound of the invention can be in the form of a “prodrug,” which includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

The compounds and compositions described herein are generally useful for the inhibition of signaling activity of one or more GPCRs. In some embodiments the GPCR inhibited by the compounds and methods of the invention is OXER1.

The presently disclosed compounds find use in inhibiting the activity of OXER1. OXER1 is a G protein-coupled receptor (GPCR) that is highly selective for 5-oxo-ETE.

Metabolites of arachidonic acid such as 5-oxo-ETE and other eicosanoids are potent chemoattractants for eosinophils and neutrophils. The actions of these eicosanoids are mediated by approximately 20 G protein-coupled receptors, resulting in a variety of both detrimental and beneficial effects on airway smooth muscle and inflammatory cells that are strongly implicated in asthma pathophysiology (Powell, Clinical Science (2021), 135, 1945-1980). Eicosanoids stimulate a variety of responses in these cells, such as actin polymerization, calcium mobilization, integrin expression and degranulation (Powell and Rokach, Progress in Lipid Research 52: 651-665 (2013)). The biological actions of 5-oxo-ETE are mediated by the highly selective OXE receptor, such as OXER1, which is expressed on a variety of inflammatory cells as well as tumor cells. Eicosanoids acting through OXE receptor, such as OXER1, elicit migration of eosinophils and neutrophils. Eosinophils are major effectors cells in the immune system. They are part of innate immune system. When activated, they release a cocktail of cytotoxic proteins and cytokines from the eosinophilic granules. Eosinophils have a key role in Type 2 inflammation-driven diseases, possibly helping to regulate the type of immune response generated. Cytokines such as IL-4, IL-13 and IL-25 are found within eosinophilic granules. They direct the Th2-polarized responses, which are linked with asthma and other allergic and inflammatory diseases. Activated eosinophil also release lipid mediators that can cause airway smooth muscle contraction and contribute to airway hyper-responsiveness. Aberrantly-activated eosinophils are known to be present in patients with severe asthma and other eosinophil-related diseases (Klion, A. D., Ackerman, S. J. & Bochner, B. S. Contributions of Eosinophils to Human Health and Disease. Annu. Rev. Pathol. Mech. Dis. 15, 179-209 (2020). Ramirez, G. A. et al. Eosinophils from Physiology to Disease: A Comprehensive Review. Biomed Res Int 2018, e9095275 (2018)).

The 5-oxo-ETE/OXER1 axis plays an important role in both cell migration and survival, and in the pathogenesis of diseases involving eosinophils and neutrophils, including a variety of inflammatory diseases and cancers. In an embodiment, the present invention provides compounds, combinations, compositions and methods as defined herein that may provide inhibition of migration of eosinophils and neutrophils. As such, treatment or prevention of disease states that may be alleviated by inhibition of eosinophil or neutrophil migration is also encompassed.

5-Oxo-ETE has been shown to play an important role in allergen-induced eosinophilia. Blocking its effects with OXER1 antagonist S-Y048, may provide a novel therapeutic approach for eosinophilic diseases (Inhibition of allergen-induced dermal eosinophilia by an oxoeicosanoid receptor antagonist in non-human primates; Miller et al, Br J Pharmacol, (2020), 177(2), 360-371). In an embodiment, the present invention provides compounds, combinations, compositions and methods as defined herein that may provide treatment for eosinophilic diseases.

Metabolomics studies 5-oxo-ETE was identified as a critical metabolite, and its receptor OXER1 significantly increased in the plasma of acute myocardial infarction (AMI) patients. 5-oxo-ETE and OXER1 seem to play an essential role in triggering myocardial ischemic injury via branched chain amino acid transaminase 1 (BCAT1), and overexpression of BCAT1/BCAT2 (branched chain amino acid transaminase 2) in the heart could ameliorate myocardial ischemic injury. Thus, therapeutic targeting OXER1 and BCAT1/BCAT2 is a promising strategy for the clinical management of AMI (Oxoeicosanoid receptor inhibition alleviates acute myocardial infarction through activation of BCAT1, Lai et al, Basic Research in Cardiology, (2021), 116:3, 1-25. In an embodiment, the present invention provides compounds, combinations, compositions and methods as defined herein that may provide a promising treatment for the clinical management of AMI.

5-Oxo-ETE/OXER1 mediated eosinophil infiltration into the lungs of asthmatics has been shown to be responsible for the late phase of inflammatory asthma. OXER1 antagonists, through preventing eosinophil migration to the lung during an asthma attack and thereby reducing asthma symptoms, may be a promising therapeutic approach for treatment of late phase of inflammatory asthma (Gore et al, J. Med. Chem., (2013), 56, 3725-3732). In an embodiment, the present invention provides compounds, combinations, compositions and methods as defined herein that may provide a promising treatment for the late phase of inflammatory asthma.

Selective OXER1 antagonists have been shouwn to inhibit allergen-induced eosinophil infiltration into the skin of rhesus monkeys that had been experimentally sensitized to house dust mite and inhibit pulmonary inflammation resulting from challenge with aerosolized allergen (Powell W.S., Rokach J., Targeting the OAE Receptor as a Potential Novel Therapy for Asthma, Biochem. Pharmacol., (2020), 179, 113930). These results provided the first evidence for a pathophysiological role for 5-oxo-ETE in mammals and suggest that selective OXE receptor antagonists could be used either on their own or in combination with current asthma medication such as glucocorticoids or cysLT1 antagonists, which act by different mechanisms, thus potentially resulting in synergy between these drugs. In an embodiment, the present invention provides compounds, combinations, compositions and methods as defined herein that may provide, on their own or in combination with a current asthma medication, a promising treatment for asthma and other eosinophilic diseases.

High OXER1 expression has been shown to be a major cause of poor prognosis in triple-negative and ER breast cancer (Masi et al., Oncogenesis, (2020), 9, 105). In an embodiment, the present invention provides compounds, combinations, compositions and methods as defined herein that may represent, through OXER1 inhibition, promising and rational agents for the personalized treatment of triple-negative and ER⁻ breast cancer.

It has been shown that 5-Oxo-ETE induces a potent migratory response, and LTB₄ elicited degranulation of basophils (Iikura et al, J Allergy Clin Imminol, (2005), 116, 578-585), suggesting that 5-oxo-ETE might afford opportunities for therapeutic targeting in allergic inflammation. In an embodiment, the present invention provides compounds, combinations, compositions and methods as defined herein that may provide, through OXER1 inhibition, a promising treatment for allergic inflammation.

5-oxo-ETE can prolong the survival of eosinophils by stimulating monocytes to release the potent survival factor granulocyte/macrophage colony-stimulating factor (GM-CSF) (Stamatiou et al, J. Biol. Chem., (2004), 279, 28159-28164). Interaction of 5-oxo-ETE with monocytes/macrophages to release GM-CSF could have important implications in diseases such as asthma, as GM-CSF is known to be very important for the survival of eosinophils once they have reached the lung. Furthermore, because of the potent effects of GM-CSF on neutrophils and monocytes, 5-oxo-ETE could also be implicated in diseases such as arthritis and atherosclerosis, which are characterized by the accumulation of neutrophils and monocytes in joints and arteries, respectively. The potent effects of 5-oxo-ETE on the migration of eosinophils, neutrophils, and monocytes, along with its survival-enhancing effects, suggest that this substance may be an important mediator in a variety of inflammatory diseases. In an embodiment, the present invention provides compounds, combinations, compositions and methods as defined herein that may provide, through OXER1 inhibition, a promising treatment for s variety of inflammatory diseases, such as asthma, arthritis, and atherosclerosis.

It has been shown that 5-oxo-ETE can stimulate proliferation of prostate tumor cells and the OXE receptor is expressed on prostate tumor cells. Metabolites of arachidonic acid including HETEs and oxo-ETEs have been shown to increase growth and promote survival of a variety of cancers, including lung, pancreatic and prostate cancer. Moreover 5-hydroxyeicosatetraenoids are the principal arachidonic acid metabolite in prostate cancer cells (see e.g. WO 2007/025254 and US 2005/0106603 for review of the role of G-protein coupled eicosanoid receptors in cancer). These findings indicate a potential role for the 5-oxo-ETE receptor antagonists of the compounds defined herein in treatment or prevention of certain cancers, as well as induction of apoptosis in these cancer cells. Thus in an embodiment, there is provided compounds, combinations, compositions and methods as defined herein that may be useful in the treatment or prevention of cancer, including lung, pancreatic and/or prostate cancer. In an aspect, there is provided herein a method that may be useful for the treatment or prevention of lung, pancreatic and/or prostate cancer. In another aspect, there is provided a method that may be useful for inducing apoptosis in a cancer cell, e.g. a lung, pancreatic and/or prostate cancer cells.

Accordingly, the compounds, combinations and compositions provided herein are useful for the treatment or prevention of diseases or conditions involving 5-oxo-ETE. Accordingly there is provided compounds, combinations, compositions and methods as defined herein that may provide treatment or prevention of eosinophilic and inflammatory conditions.

There are many diseases or conditions that are inflammatory in their nature. For example, inflammatory diseases that affect the population include asthma, severe eosinophilic asthma, chronic obstructive pulmonary disease (COPD), hypereosinophilic syndrome (HES), nasal polyposis, allergic rhinitis, atopic dermatitis, chronic spontaneous urticaria, psoriasis, acne, idiopathic pulmonary fibrosis, eosinophilic gastritis, eosinophilic esophagitis (EoE), and eosinophilic gastroenteritis. Inflammation is also a common cause of pain. Inflammatory pain may arise for numerous reasons, such as infection, surgery or other trauma. The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions. The term “inflammation” will thus also be understood to include any inflammatory disease, disorder or condition per se, any condition that has an inflammatory component associated with it, and/or any condition characterised by inflammation as a symptom, insofar as it is related to a respiratory disease or condition, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

In an aspect, there is provided compounds, combinations, compositions and methods as defined herein that may provide treatment or prevention of respiratory disease or condition such as asthma, chronic obstructive pulmonary disease, pulmonary fibrosis, allergic rhinitis, rhinitis, and any other respiratory disease or condition with an inflammatory component, characterized by inflammation or characterized by eosinophilia.

In one embodiment, there is provided compounds, combinations, compositions and methods as defined herein that may provide treatment or prevention of asthma, which method comprises administration of a compound or composition of the disclosure to a subject.

Asthma is a common chronic disorder of the airways that is complex and characterized by variable and recurring symptoms including airflow obstruction, bronchoconstric-tion and an underlying inflammation. Treatment regimens for asthma vary depending on the severity of the condition.

As used herein, the term “asthma” includes all types of asthma, including without limitation: mild, moderate and severe asthma; exercise-induced asthma; aspirin-induced asthma; extrinsic or allergic asthma; intrinsic or non-allergic asthma; occupational asthma; cough-variant asthma; nocturnal asthma; child-onset asthma; and adult-onset asthma.

In one embodiment, there is provided compounds, combinations, compositions and methods as defined herein that may provide treatment or prevention of chronic obstructive pulmonary disease (COPD). COPD refers to a group of diseases of the lungs in which the airways become narrowed, typically due to an abnormal inflammatory response in the lungs. Non-limiting examples of COPD include bronchitis and emphysema. Idiopathic pulmonary fibrosis (IPF) is another lung disease also involving eicosanoids.

In one embodiment, there is provided compounds, combinations, compositions and methods as defined herein that may provide treatment or prevention of allergic rhinitis. Allergic rhinitis is an inflammation of the nasal passages, usually associated with watery nasal discharge and itching of the nose and eyes. Allergies occur when the immune system overreacts to particles in the air and produces an allergic reaction.

In accordance with another aspect, there is provided compounds, combinations, compositions and methods as defined herein that may provide treatment or prevention of a disease or condition involving eicosanoids such as 5-oxo-ETE and 5-HETE.

In accordance with another aspect, there is provided compounds, combinations, compositions and methods as defined herein that may be useful for inhibiting the effect of eicosanoids such as 5-oxo-ETE and 5-HETE and 5-oxo-15-HETE.

In accordance with another aspect, there is provided compounds, combinations, compositions and methods as defined herein that may be useful for antagonizing the 5-oxo-ETE receptors, such as the OXE receptor.

It should be understood that, in addition to blocking biological responses to 5-oxo-ETE, 5-oxo-15-HETE and 5-HETE, the compounds and compositions of the invention may block biological responses to other related eicosanoids which can also act as ligands for the OXE receptor. Thus “eicosanoid”, as used herein, means a substance derived from a fatty acid having 20 carbon atoms, such as eicosanoic acid, and in an aspect, a fatty acid in which the 8th position is unsaturated. Non-limiting examples of eicosanoids which are encompassed in the methods presented herein include 5-oxo-ETE, 5-HETE, 5-HIPETE, arachidonic acid, 5-oxo-ETrE (5-oxo-6E,8Z,11Z-eicosatrienoic acid), 5-HETrE (5-hydroxy-6E,8Z,11Z-eicosatrienoic acid), eicosa-5Z, 8Z, 11Z-trienoic acid, 5-oxo-EDE (5-oxo-6E,8Z-eicosadienoic acid), and eicosa-5Z,8Z-dienoic acid. In addition, certain 18-carbon polyunsaturated fatty acids are included, e.g. 5-oxo-ODE (5-oxo-6E,8Z-octadecadienoic acid), 5-HODE (5-hydroxy-6E,8Z-octadecadienoic acid), and sebaleic acid (5Z,8Z-octadecadienoic acid).

In an aspect, there is provided compounds, combinations, compositions and methods as defined herein that may be useful in the treatment or prevention of viral infections (e.g. influenza, common cold).

In an aspect, there is provided compounds, combinations, compositions and methods as defined herein for that may be useful in the treatment or prevention of atopic dermatitis, psoriasis and/or acne. [00227]5-LO products have been considered a factor in the development of tissue inflammation. Synthesis of leukot-rienes and 5 oxo-ETE is controlled by the enzyme 5-lipoxygenase.

The pharmacologic role of 5-LO products has been inves-15 tigated in psoriasis. It has been suggested that the inhibition of 5-LO products may be useful in the treatment of psoriasis. Tissue inflammation is a component of the acne process. Therefore, inhibitors of 5-lipoxygenase products may be useful compounds in the treatment of acne vulgaris.

Atopic dermatitis is a chronic, relapsing skin condition. The pathophysiology is believed to involve the release of inflammatory mediators. 5-LO products are believed to play a role in inflammatory and atopic conditions. Modulators of 5-LO products may therefore be useful in the treatment of atopic dermatitis.

In one embodiment, the subject matter disclosed herein is directed to a method of inhibiting OXER1, the method comprising contacting OXER1 with an effective amount of a compound of the invention or a pharmaceutical composition described herein.

In certain embodiments, the subject matter disclosed herein is directed to a method for modulating an immune response in a subject in need thereof, wherein the method comprises administering to the subject an effective amount of a compound of the invention or a pharmaceutical composition described herein.

The presently disclosed compounds bind directly to OXER1 and inhibit its signaling activity. In some embodiments, the presently disclosed compounds reduce, inhibit, or otherwise diminish the OXER1-mediated inflammatory response.

The presently disclosed compounds may or may not be a specific OXER1 antagonist. A specific OXER1 antagonist reduces the biological activity of OXER1 by an amount that is statistically greater than the inhibitory effect of the antagonist on any other protein (e.g., other GPCRs). In certain embodiments, the presently disclosed compounds specifically inhibit the signaling activity of OXER1. In some of these embodiments, the IC₅₀ of the OXER1 antagonist for OXER1 is about 90%, 80%, 70%, 60%, 50%, 40%, 30%, 20%, 10%, 0.1%, 0.01%, 0.001%, or less of the IC₅₀ of the OXER1 antagonist for another GPCR activated by free fatty acids (FFA) or other type of GPCR (e.g., Class A GPCR).

The presently disclosed compounds can be used in a method for inhibiting OXER1. Such methods comprise contacting OXER1 with an effective amount of a presently disclosed compound. By “contact” is intended bringing the compound within close enough proximity to an isolated OXER1 GPCR or a cell expressing OXER1 such that the compound is able to bind to and inhibit the activity of OXER1. The compound can be contacted with OXER1 in vitro or in vivo via administration of the compound to a subject.

Any method known in the art to measure the signaling activity of OXER1 may be used to determine if OXER1 has been inhibited, including in vitro assays or the measurement of a downstream biological effect of OXER1 signaling activity.

The presently disclosed compounds can be used to treat a OXER1-dependent disorder. As used herein, a “OXER1-dependent disorder” is a pathological condition in which OXER1 activity is necessary for the genesis or maintenance of the pathological condition. In some embodiments, the OXER1-dependent disorder is an inflammatory condition.

The presently disclosed compounds also find use in modulating an immune response in a subject in need thereof. Such methods comprise administering an effective amount of a compound of the invention.

As used herein, “modulating an immune response” refers to modulation of any immunogenic response to an antigen.

In another aspect of the invention, this invention provides novel compounds of the invention for use in therapy.

In additional aspects, this invention provides methods for synthesizing a compound of the invention, with representative synthetic protocols and pathways disclosed herein.

Accordingly, it is a principal object of this invention to provide a compound of the invention, which can modify the activity of OXER1 and thus prevent or treat any conditions that may be causally related thereto.

Other objects and advantages will become apparent to those skilled in the art from a consideration of the ensuing detailed description.

The present disclosure provides methods of treating a OXER1-mediated disorder, disease, or condition in a patient comprising administering to said patient in need thereof the compound described herein (e.g., a compound of Formula I) or a pharmaceutical composition comprising same. In some embodiments, the disease, disease, or condition is asthma, severe eosinophilic asthma, late phase of inflammatory asthma, chronic obstructive pulmonary disease (COPD), hypereosinophilic syndrome (HES), nasal polyposis, allergic inflammation, allergic rhinitis, atopic dermatitis, chronic spontaneous urticaria, psoriasis, acne, idiopathic pulmonary fibrosis, eosinophilic gastritis, eosinophilic esophagitis (EoE), eosinophilic gastroenteritis, arthritis, atherosclerosis, or acute myocardial infarction. In some embodiments, the disease, disease, or condition is asthma.

The present disclosure provides methods of modulating (e.g., inhibiting) OXER1 activity, said method comprising administering to a patient a compound provided herein, or an N-oxide, or a pharmaceutically acceptable salt thereof.

In one aspect, provided herein is a method for treating of cancer in a subject in need thereof comprising administering to the subject an effective amount of a compound of the invention or a pharmaceutically acceptable salt, prodrug, metabolite, or derivative thereof.

In the methods described herein, a compound of the invention or a pharmaceutical composition thereof is administered to a subject that has cancer.

In certain embodiments, the subject matter disclosed herein is directed to a method for treating a OXER1-dependent disorder, the method comprising administering to a subject in need thereof an effective amount of a compound of the invention or a pharmaceutical composition described herein. In certain aspects of this embodiment, the OXER1-dependent disorder is a cancer.

In some embodiments, the subject matter disclosed herein is directed to a method for treatment of chronic viral infections. In some embodiments, the subject matter disclosed herein is directed to the use of an OXER1 inhibitor as an adjuvant treatment for increasing the efficacy of vaccination.

In some embodiments, the invention provides a pharmaceutical composition comprising an effective amount of a compound of the invention, or a pharmaceutically acceptable salt, hydrate, solvate, or prodrug thereof, and a pharmaceutically acceptable carrier.

In certain aspects, the invention provides a method of treating cell proliferation disorders, including cancers.

In one aspect, the invention provides a method of treating a cell proliferation disorder in a subject, comprising administering a therapeutically effective amount of a compound of the invention, or a pharmaceutically acceptable salt, hydrate, solvate, or prodrug thereof, to the subject.

In certain embodiments, the cell proliferation disorder is cancer.

Examples of cancers that are treatable using the compounds of the present disclosure include, but are not limited to, chronic or acute leukemias including acute myeloid leukemia, chronic myeloid leukemia, acute lymphoblastic leukemia, chronic lymphocytic leukemia, solid tumors of childhood, lymphocytic lymphoma, and combinations of said cancers.

In some embodiments, cancers that are treatable using the compounds of the present disclosure include, but are not limited to, hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, and combinations of said cancers.

In certain embodiments, the cancer is leukemia. In another embodiment the cancer is selected from the group consisting of acute myeloid leukemia and chronic myelogenous leukemia.

In certain embodiments, the cancer is selected from leukemia and cancers of the blood. In particular embodiments, the cancer is present in an adult patient; in additional embodiments, the cancer is present in a pediatric patient. In particular embodiments, the cancer is AIDS-related.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In some embodiments, diseases and indications that are treatable using the compounds of the present disclosure include, but are not limited to hematological cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET)), myelodysplasia syndrome (MDS), T-cell acute lymphoblastic lymphoma (T-ALL), multiple myeloma, cutaneous T-cell lymphoma, Waldenstrom's Macroglobulinemia, hairy cell lymphoma, chronic myelogenic lymphoma and Burkitt's lymphoma.

As used herein the term ‘inflammatory condition(s)’ refers to the group of conditions including inflammatory bowel diseases (IBD) (e.g., Crohn's disease, ulcerative colitis), rheumatoid arthritis, vasculitis, lung diseases (e.g., chronic obstructive pulmonary disease (COPD) and lung interstitial diseases (e.g., idiopathic pulmonary fibrosis (IPF))), psoriasis, gout, allergic airway disease (e.g., asthma, rhinitis), and endotoxin-driven disease states (e.g., complications after bypass surgery or chronic endotoxin states contributing to e.g., chronic cardiac failure). Particularly the term refers to rheumatoid arthritis, allergic airway disease (e.g., asthma) and inflammatory bowel diseases. In a further particular aspect, the term refers to uveitis, periodontitis, esophagitis, neutrophilic dermatoses (e.g., pyoderma gangrenosum, Sweet's syndrome), severe asthma, and skin and/or colon inflammation caused by oncology treatments aimed at activating the immune response.

As used herein the term ‘pain’ refers to diseases or disorders characterized by unpleasant feeling often caused by intense or damaging stimuli, and include but is not limited to nociceptive pain, inflammatory pain (associated with tissue damage and inflammatory cell infiltration) and neuropathic or dysfunctional pain (caused by damage to or abnormal function of the nervous system), and/or pain associated or caused by the conditions mentioned herein. Pain can be acute or chronic.

As used herein the term ‘leukemia’ refers to neoplastic diseases of the blood and blood forming organs. Such diseases can cause bone marrow and immune system dysfunction, which renders the host highly susceptible to infection and bleeding. In particular the term leukemia refers to acute myeloid leukaemia (AML) and acute lymphoblastic leukemia (ALL).

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

The presently disclosed compounds may be administered in any suitable manner known in the art. In some embodiments, the compound of the invention or a pharmaceutically acceptable salt, prodrug, metabolite, or derivative thereof is administered intravenously, intramuscularly, subcutaneously, topically, orally, transdermally, intraperitoneally, intraorbitally, by implantation, by inhalation, intrathecally, intraventricularly, intratumorally, or intranasally.

In some embodiments, the OXER1 antagonist is administered continuously. In other embodiments, the OXER1 antagonist is administered intermittently. Moreover, treatment of a subject with an effective amount of a OXER1 antagonist can include a single treatment or can include a series of treatments.

It is understood that appropriate doses of the active compound depends upon a number of factors within the knowledge of the ordinarily skilled physician or veterinarian. The dose(s) of the active compound will vary, for example, depending upon the age, body weight, general health, gender, and diet of the subject, the time of administration, the route of administration, the rate of excretion, and any drug combination.

It will also be appreciated that the effective dosage of a compound of the invention or a pharmaceutically acceptable salt, prodrug, metabolite, or derivative thereof used for treatment may increase or decrease over the course of a particular treatment. Changes in dosage may result and become apparent from the results of diagnostic assays.

In some embodiments, the OXER1 antagonist is administered to the subject at a dose of between about 0.001 μg/kg and about 1000 mg/kg, including but not limited to about 0.001 μg/kg, 0.01 μg/kg, 0.05 μg/kg, 0.1 μg/kg, 0.5 μg/kg, 1 μg/kg, 10 μg/kg, 25 μg/kg, 50 μg/kg, 100 μg/kg, 250 μg/kg, 500 μg/kg, 1 mg/kg, 5 mg/kg, 10 mg/kg, 25 mg/kg, 50 mg/kg, 100 mg/kg, and 200 mg/kg.

In the methods described herein, the method can further comprise administering a chemotherapeutic agent to the subject. In certain aspects of this embodiment, the chemotherapeutic agent is administered to the subject simultaneously with the compound or the composition. In certain aspects of this embodiment, the chemotherapeutic agent is administered to the subject prior to administration of the compound or the composition. In certain aspects of this embodiment, the chemotherapeutic agent is administered to the subject after administration of the compound or the composition.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “subject” refers to animals such as mammals, including, but not limited to, primates (e.g., humans), cows, sheep, goats, horses, dogs, cats, rabbits, rats, mice and the like. In certain embodiments, the subject is a human.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

A compound of the invention may be used as a therapeutic agent for the treatment of conditions in mammals that are causally related or attributable to aberrant activity of OXER1 and/or aberrant OXER1 expression and/or aberrant OXER1 distribution.

Accordingly, a compound and pharmaceutical compositions of the invention find use as therapeutics for the prophylaxis and/or treatment of inflammatory conditions, pain, neuroinflammatory conditions, neurodegenerative conditions, infectious diseases, autoimmune diseases, endocrine and/or metabolic diseases, cardiovascular diseases, leukemia, and/or diseases involving impairment of immune cell functions, in mammals including humans.

Accordingly, in one aspect, the present invention provides the compound of the invention, or a pharmaceutical composition comprising the compound of the invention for use as a medicament.

In another aspect, the present invention provides the compound of the invention, or a pharmaceutical composition comprising the compound of the invention for use in the manufacture of a medicament.

In yet another aspect, the present invention provides a method of treating a mammal having, or at risk of having a disease disclosed herein. In a particular aspect, the present invention provides a method of treating a mammal having, or at risk of having inflammatory conditions, pain, neuroinflammatory conditions, neurodegenerative conditions, infectious diseases, autoimmune diseases, endocrine and/or metabolic diseases, cardiovascular diseases, leukemia, and/or diseases involving impairment of immune cell functions, in mammals including humans, said method comprising administering an effective amount of a compound of the invention, or one or more of the pharmaceutical compositions herein described.

In one aspect, the present invention provides the compound of the invention, or a pharmaceutical composition comprising the compound of the invention for use in the prophylaxis and/or treatment of inflammatory conditions. In a specific embodiment, the inflammatory condition is selected from inflammatory bowel disease (IBD), rheumatoid arthritis, vasculitis, chronic obstructive pulmonary disease (COPD), and idiopathic pulmonary fibrosis (IPF). In another specific embodiment, the inflammatory condition is selected from uveitis, periodontitis, esophagitis, neutrophilic dermatoses (e.g., pyoderma gangrenosum, Sweet's syndrome), severe asthma, and skin and/or colon inflammation caused by oncology treatments aimed at activating the immune response.

In another aspect, the present invention provides the compound of the invention, or a pharmaceutical composition comprising the compound of the invention for use in the manufacture of a medicament for the prophylaxis and/or treatment of inflammatory conditions. In a specific embodiment, the inflammatory condition is selected from inflammatory bowel disease (IBD), rheumatoid arthritis, vasculitis, chronic obstructive pulmonary disease (COPD), and idiopathic pulmonary fibrosis (IPF). In another specific embodiment, the inflammatory condition is selected from uveitis, periodontitis, esophagitis, neutrophilic dermatoses (e.g., pyoderma gangrenosum, Sweet's syndrome), severe asthma, and skin and/or colon inflammation caused by oncology treatments aimed at activating the immune response.

In another aspect, the present invention provides a method of treating a mammal having, or at risk of having a disease selected from inflammatory conditions (for example inflammatory bowel diseases (IBD), rheumatoid arthritis, vasculitis), lung diseases (e.g., chronic obstructive pulmonary disease (COPD) and lung interstitial diseases (e.g., idiopathic pulmonary fibrosis (IPF))), neuroinflammatory conditions, infectious diseases, autoimmune diseases, endocrine and/or metabolic diseases, and/or diseases involving impairment of immune cell functions, which method comprises administering an effective amount of a compound of the invention, or one or more of the pharmaceutical compositions herein described.

In additional method of treatment aspects, this invention provides methods of treatment and/or prophylaxis of a mammal susceptible to or afflicted with inflammatory conditions, which method comprises administering an effective amount of a compound of the invention, or one or more of the pharmaceutical compositions herein described. In a specific embodiment, the inflammatory condition is selected from inflammatory bowel disease (IBD), rheumatoid arthritis, vasculitis, chronic obstructive pulmonary disease (COPD), and idiopathic pulmonary fibrosis (IPF). In another specific embodiment, the inflammatory condition is selected from uveitis, periodontitis, esophagitis, neutrophilic dermatoses (e.g., pyoderma gangrenosum, Sweet's syndrome), severe asthma, and skin and/or colon inflammation caused by oncology treatments aimed at activating the immune response.

In one aspect, the present invention provides the compound of the invention, or a pharmaceutical composition comprising the compound of the invention for use in the prophylaxis and/or treatment of pain. In a specific embodiment, the pain is acute or chronic and is selected from nociceptive pain, inflammatory pain, and neuropathic or dysfunctional pain.

In another aspect, the present invention provides the compound of the invention, or a pharmaceutical composition comprising the compound of the invention for use in the manufacture of a medicament for the prophylaxis and/or treatment of pain. In a specific embodiment, the pain is acute or chronic and is selected from nociceptive pain, inflammatory pain, and neuropathic or dysfunctional pain.

In additional method of treatment aspects, this invention provides methods of treatment and/or prophylaxis of a mammal susceptible to or afflicted with pain, which method comprises administering an effective amount of a compound of the invention, or one or more of the pharmaceutical compositions herein described. In a specific embodiment, the pain is acute or chronic and is selected from nociceptive pain, inflammatory pain, and neuropathic or dysfunctional pain.

In one aspect, the present invention provides a compound of the invention, or a pharmaceutical composition comprising the compound of the invention for use in the prophylaxis and/or treatment of neuroinflammatory conditions, Guillain-Barre syndrome (GBS), multiple sclerosis, axonal degeneration, autoimmune encephalomyelitis.

In another aspect, the present invention provides a compound of the invention, or a pharmaceutical composition comprising the compound of the invention for use in the manufacture of a medicament for the prophylaxis and/or treatment of neuroinflammatory conditions, Guillain-Barre syndrome (GBS), multiple sclerosis, axonal degeneration, autoimmune encephalomyelitis.

In additional method of treatment aspects, this invention provides methods of treatment and/or prophylaxis of a mammal susceptible to or afflicted with neuroinflammatory conditions, Guillain-Barre syndrome (GBS), multiple sclerosis, axonal degeneration, autoimmune encephalomyelitis, which method comprises administering an effective amount of a compound of the invention, or one or more of the pharmaceutical compositions herein described.

In one aspect, the present invention provides a compound of the invention, or a pharmaceutical composition comprising the compound of the invention for use in the prophylaxis and/or treatment of infectious disease(s). In a specific embodiment, the infectious disease(s) is selected from sepsis, septicemia, endotoxemia, systemic inflammatory response syndrome (SIRS), gastritis, enteritis, enterocolitis, tuberculosis, and other infections involving, for example, Yersinia, Salmonella, Chlamydia, Shigella, enterobacteria species.

In another aspect, the present invention provides a compound of the invention, or a pharmaceutical composition comprising the compound of the invention for use in the manufacture of a medicament for the prophylaxis and/or treatment of infectious disease(s). In a specific embodiment, the infectious disease(s) is selected from sepsis, septicemia, endotoxemia, systemic inflammatory response syndrome (SIRS), gastritis, enteritis, enterocolitis, tuberculosis, and other infections involving, for example, Yersinia, Salmonella, Chlamydia, Shigella, enterobacteria species.

In additional method of treatment aspects, this invention provides methods of treatment and/or prophylaxis of a mammal susceptible to or afflicted with infectious disease(s), which method comprises administering an effective amount of a compound of the invention, or one or more of the pharmaceutical compositions herein described. In a specific embodiment, the infectious disease is selected from sepsis, septicemia, endotoxemia, systemic inflammatory response syndrome (SIRS), gastritis, enteritis, enterocolitis, tuberculosis, and other infections involving, for example, Yersinia, Salmonella, Chlamydia, Shigella, enterobacteria species.

In one aspect, the present invention provides the compound of the invention, or a pharmaceutical composition comprising the compound of the invention for use in the prophylaxis and/or treatment of autoimmune diseases, and/or diseases involving impairment of immune cell functions. In a specific embodiment, the autoimmune diseases and/or diseases involving impairment of immune cell functions is selected from COPD, asthma, psoriasis, systemic lupus erythematosis, type I diabetes mellitus, vasculitis and inflammatory bowel disease.

In another aspect, the present invention provides the compound of the invention, or a pharmaceutical composition comprising the compound of the invention for use in the manufacture of a medicament for the prophylaxis and/or treatment of autoimmune diseases and/or diseases involving impairment of immune cell functions. In a specific embodiment, the autoimmune diseases, and/or diseases involving impairment of immune cell functions is selected from COPD, asthma, psoriasis, systemic lupus erythematosis, type I diabetes mellitus, vasculitis and inflammatory bowel disease.

In additional method of treatment aspects, this invention provides methods of treatment and/or prophylaxis of a mammal susceptible to or afflicted with autoimmune diseases and/or diseases involving impairment of immune cell functions, which method comprises administering an effective amount of a compound of the invention, or one or more of the pharmaceutical compositions herein described. In a specific embodiment, the autoimmune diseases and/or diseases involving impairment of immune cell functions is selected from COPD, asthma, psoriasis, systemic lupus erythematosis, type I diabetes mellitus, vasculitis and inflammatory bowel disease.

In one aspect, the present invention provides the compound of the invention, or a pharmaceutical composition comprising the compound of the invention for use in the prophylaxis and/or treatment of endocrine and/or metabolic diseases. In a specific embodiment, the endocrine and/or metabolic diseases is selected from hypothyroidism, congenital adrenal hyperplasia, diseases of the parathyroid gland, diabetes mellitus, diseases of the adrenal glands (including Cushing's syndrome and Addison's disease), ovarian dysfunction (including polycystic ovary syndrome), cystic fibrosis, phenylketonuria (PKU), diabetes, hyperlipidemia, gout, and rickets.

In another aspect, the present invention provides the compound of the invention, or a pharmaceutical composition comprising the compound of the invention for use in the manufacture of a medicament for the prophylaxis and/or treatment of endocrine and/or metabolic diseases. In a specific embodiment, the endocrine and/or metabolic diseases is selected from hypothyroidism, congenital adrenal hyperplasia, diseases of the parathyroid gland, diabetes mellitus, diseases of the adrenal glands (including Cushing's syndrome and Addison's disease), ovarian dysfunction (including polycystic ovary syndrome), cystic fibrosis, phenylketonuria (PKU), diabetes, hyperlipidemia, gout, and rickets.

In additional method of treatment aspects, this invention provides methods of treatment and/or prophylaxis of a mammal susceptible to or afflicted with endocrine and/or metabolic diseases, which method comprises administering an effective amount of a compound of the invention, or one or more of the pharmaceutical compositions herein described. In a specific embodiment, the endocrine and/or metabolic diseases is selected from hypothyroidism, congenital adrenal hyperplasia, diseases of the parathyroid gland, diabetes mellitus, diseases of the adrenal glands (including Cushing's syndrome and Addison's disease), ovarian dysfunction (including polycystic ovary syndrome), cystic fibrosis, phenylketonuria (PKU), diabetes, hyperlipidemia, gout, and rickets.

As a further aspect of the invention there is provided a compound of the invention for use as a medicament especially in the treatment or prevention of the aforementioned conditions and diseases. Also provided herein is the use of the compound in the manufacture of a medicament for the treatment or prevention of one of the aforementioned conditions and diseases.

A particular regimen of the present method comprises the administration to a subject in suffering from an inflammatory condition, of an effective amount of a compound of the invention for a period of time sufficient to reduce the level of inflammation in the subject, and preferably terminate, the processes responsible for said inflammation. A special embodiment of the method comprises administering of an effective amount of a compound of the invention to a subject suffering from or susceptible to the development of inflammatory condition, for a period of time sufficient to reduce or prevent, respectively, inflammation of said patient, and preferably terminate, the processes responsible for said inflammation.

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 2 g/day for a 40 to 80 kg human patient.

Transdermal doses are generally selected to provide similar or lower blood levels than are achieved using injection doses.

When used to prevent the onset of a condition, a compound of the invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

A compound of the invention can be administered as the sole active agent or it can be administered in combination with other therapeutic agents, including other compounds that demonstrate the same or a similar therapeutic activity, and that are determined to be safe and efficacious for such combined administration. In a specific embodiment, co-administration of two (or more) agents allows for significantly lower doses of each to be used, thereby reducing the side effects seen.

In one embodiment, a compound of the invention is co-administered with another therapeutic agent for the treatment and/or prevention of an inflammatory condition; particular agents include, but are not limited to, immunoregulatory agents e.g., azathioprine, corticosteroids (e.g., prednisolone or dexamethasone), cyclophosphamide, cyclosporin A, tacrolimus, Mycophenolate Mofetil, muromonab-CD3 (OKT3, e.g., Orthocolone®), ATG, aspirin, acetaminophen, ibuprofen, naproxen, and piroxicam.

In one embodiment, a compound of the invention is co-administered with another therapeutic agent for the treatment and/or prevention of arthritis (e.g., rheumatoid arthritis); particular agents include but are not limited to analgesics, non-steroidal anti-inflammatory drugs (NSAIDS), steroids, synthetic DMARDS (for example but without limitation methotrexate, leflunomide, sulfasalazine, auranofin, sodium aurothiomalate, penicillamine, chloroquine, hydroxychloroquine, azathioprine, and cyclosporin), and biological DMARDS (for example but without limitation Infliximab, Etanercept, Adalimumab, Rituximab, Golimumab, Certolizumab pegol, Tocilizumab, Interleukin 1 blockers and Abatacept).

In one embodiment, a compound of the invention is co-administered with another therapeutic agent for the treatment and/or prevention of autoimmune diseases; particular agents include but are not limited to: glucocorticoids, cytostatic agents (e.g., purine analogs), alkylating agents, (e.g., nitrogen mustards (cyclophosphamide), nitrosoureas, platinum compounds, and others), antimetabolites (e.g., e.g., methotrexate, azathioprine and mercaptopurine), cytotoxic antibiotics (e.g., e.g., dactinomycin anthracyclines, mitomycin C, bleomycin, and mithramycin), antibodies (e.g., anti-CD20, anti-CD25 or anti-CD3 (OTK3) monoclonal antibodies, Atgam® and Thymoglobuline®), cyclosporin, tacrolimus, rapamycin (sirolimus), interferons (e.g., IFN-β), TNF binding proteins (e.g., infliximab (Remicade®), etanercept (Enbrel®), or adalimumab (Humira®)), mycophenolate, Fingolimod, and Myriocin.

In one embodiment, a compound of the invention is co-administered with another therapeutic agent for the treatment and/or prevention of infectious diseases; particular agents include but are not limited to antibiotics. In a particular embodiment, a compound of the invention is co-administered with another therapeutic agent for the treatment and/or prevention of infections of any organ of the human body; particular agents include but are not limited to: aminoglycosides, ansamycins, carbacephem, carbapenems, cephalosporins, glycopeptides, lincosamides, macrolides, monobactams, nitrofurans, penicillins, polypeptides, quinolones, sulfonamides, tetracyclins, anti-mycobacterial agents, as well as chloramphenicol, fosfomycin, linezolid, metronidazole, mupirocin, rifamycin, thiamphenicol and tinidazole.

In one embodiment, a compound of the invention is co-administered with another therapeutic agent for the treatment and/or prevention of vasculitis, particular agents include but are not limited to steroids (for example prednisone, prednisolone), cyclophosphamide and eventually antibiotics in case of cutaneous infections (for example cephalexin).

In one embodiment, a compound of the invention is co-administered with another therapeutic agent for the treatment and/or prevention of esophagitis; particular agents include but are not limited to: anti-acids (e.g., formulations containing aluminum hydroxide, magnesium hydroxide, and/or simethicone), H2-antagonists (e.g., cimetidine, ranitidine, famotidine), proton pump inhibitors (e.g., omeprazole, esomeprazole, lansoprazole, rabeprazole, pantoprazole), and glucocorticoids (e.g., prednisone, budesonide).

In one embodiment, a compound of the invention is co-administered with another therapeutic agent for the treatment and/or prevention of IPF, particular agents include but are not limited to pirfenidone and bosentan.

In one embodiment, a compound of the invention is co-administered with another therapeutic agent for the treatment and/or prevention of asthma and/or rhinitis and/or COPD; particular agents include but are not limited to: beta2-adrenoceptor agonists (e.g., salbutamol, levalbuterol, terbutaline and bitolterol), epinephrine (inhaled or tablets), anticholinergics (e.g., ipratropium bromide), glucocorticoids (oral or inhaled) Long-acting 02-agonists (e.g., salmeterol, formoterol, bambuterol, and sustained-release oral albuterol), combinations of inhaled steroids and long-acting bronchodilators (e.g., fluticasone/salmeterol, budesonide/formoterol), leukotriene antagonists and synthesis inhibitors (e.g., montelukast, zafirlukast and zileuton), inhibitors of mediator release (e.g., cromoglycate and ketotifen), phosphodiesterase-4 inhibitors (e.g., Roflumilast), biological regulators of IgE response (e.g., omalizumab), antihistamines (e.g., ceterizine, cinnarizine, fexofenadine), and vasoconstrictors (e.g., oxymethazoline, xylomethazoline, nafazoline and tramazoline).

Additionally, a compound of the invention may be administered in combination with emergency therapies for asthma and/or COPD, such therapies include oxygen or heliox administration, nebulized salbutamol or terbutaline (optionally combined with an anticholinergic (e.g., ipratropium), systemic steroids (oral or intravenous, e.g., prednisone, prednisolone, methylprednisolone, dexamethasone, or hydrocortisone), intravenous salbutamol, non-specific beta-agonists, injected or inhaled (e.g., epinephrine, isoetharine, isoproterenol, metaproterenol), anticholinergics (IV or nebulized, e.g., glycopyrrolate, atropine, ipratropium), methylxanthines (theophylline, aminophylline, bamiphylline), inhalation anesthetics that have a bronchodilatory effect (e.g., isoflurane, halothane, enflurane), ketamine, and intravenous magnesium sulfate.

In one embodiment, a compound of the invention is co-administered with another therapeutic agent for the treatment and/or prevention of inflammatory bowel disease (IBD); particular agents include but are not limited to: glucocorticoids (e.g., prednisone, budesonide) synthetic disease modifying, immunomodulatory agents (e.g., methotrexate, leflunomide, sulfasalazine, mesalazine, azathioprine, 6-mercaptopurine and ciclosporin) and biological disease modifying, immunomodulatory agents (infliximab, adalimumab, rituximab, and abatacept).

In one embodiment, a compound of the invention is co-administered with another therapeutic agent for the treatment and/or prevention of pain, such as non-narcotic and narcotic analgesics; particular agents include but are not limited to: paracetamol, acetylsalicylic acid, NSAID's, codeine, dihydrocodeine, tramadol, pentazocine, pethidine, tilidine, buprenorfine, fentanyl, hydromorfon, methadon, morfine, oxycodon, piritramide, tapentadol or combinations thereof.

Course of treatment for leukemia comprises chemotherapy, biological therapy, targeted therapy, radiation therapy, bone marrow transplantation and/or combinations thereof.

Examples of further therapeutic agents for Acute Lymphoblastic Leukemia (ALL) comprise methotrexate, nelarabine, asparaginase Erwinia chrysanthemi, blinatumomab, daunorubicin, clofarabine, cyclophosphamide, cytarabine, dasatinib, doxorubicin, imatinib, ponatinib vincristine, mercaptopurine, pegaspargase, and/or prednisone.

Examples of further therapeutic agents for Acute Myeloid Leukemia (AML) comprise arsenic trioxide, daunorubicin, cyclophosphamide, cytarabine, doxorubicin, idarubicin, mitoxantrone, and/or vincristine.

Examples of further therapeutic agents for Chronic Lymphocytic Leukemia (CLL) comprise alemtuzumab, chlorambucil, ofatumumab, bendamustine, cyclophosphamide, fludarabine, obinutuzumab, ibrutinib, idelalisib, mechlorethamine, prednisone, and/or rituximab.

Examples of further therapeutic agents for Chronic Myelogenous Leukemia (CML) comprise bosutinib, busulfan, cyclophosphamide, cytarabine, dasatinib, imatinib, ponatinib, mechlorethamine, nilotinib, and/or omacetaxine.

Examples of further therapeutic agents for Hairy Cell Leukemia comprise cladiribine, pentostatin, and/or interferon alfa-2b.

By co-administration is included any means of delivering two or more therapeutic-agents to the patient as part of the same treatment regime, as will be apparent to the skilled person. Whilst the two or more agents may be administered simultaneously in a single formulation this is not essential. The agents may be administered in different formulations and at different times.

In one embodiment, a compound of the invention is co-administered with one or more further therapeutic agents for the treatment and/or prophylaxis of a fibrotic disease. In a particular embodiment, a compound of the invention is co-administered with one or two further therapeutic agents for the treatment and/or prophylaxis of a fibrotic disease. In a more particular embodiment, a compound of the invention is co-administered with one further therapeutic agent for the treatment and/or prophylaxis of a fibrotic disease.

In one embodiment, the further therapeutic agent for the treatment and/or prophylaxis of a fibrotic disease include, but are not limited to 5-methyl-1-phenyl-2-(1H)-pyridone (pirfenidone); nintedanib (Ofev® or Vargatef®); STX-100 (ClinicalTrials.gov Identifier NCT01371305), FG-3019 (ClinicalTrials.gov Identifier NCT01890265), lebrikizumab (CAS n #953400-68-5); tralokinumab (CAS n #1044515-88-9), CC-90001 (ClinicalTrials.gov Identifier NCT03142191), tipelukast (MN-001; ClinicalTrials.gov Identifier NCT02503657), ND-L⁰²-s0201 (ClinicalTrials.gov Identifier NCT03538301), KD025 (ClinicalTrials.gov Identifier NCT02688647), TD139 (ClinicalTrials.gov Identifier NCT02257177), VAY736 (ClinicalTrials.gov Identifier NCT03287414), PRM-151 (ClinicalTrials.gov Identifier NCT02550873) and PBI-4050 (ClinicalTrials.gov Identifier NCT02538536). In a particular embodiment, the further therapeutic agent for the treatment and/or prophylaxis of a fibrotic disease is an autotaxin (or ectonucleotide pyrophosphatase/phosphodiesterase 2 or NPP2 or ENPP2) inhibitor, examples of which are described in WO 2014/139882, such as GLPG1690.

In one embodiment, a compound of the invention is co-administered with another therapeutic agent for the treatment and/or prophylaxis of NASH, particular agents include but are not limited to weight loss treatment agents (for example Sibutramine, or Orlistat), insulin-sensitizing agents (for example Metformin, Thiazolidinedione, Rosiglitazone, or Pioglitazone), lipid-lowering agents (for example Gemfibrozil), Antioxidants (for example Vitamine E, N-acetylcysteine, Betaine, or Pentoxifylline), Angiotensin-converting enzyme inhibitors, Angiotensin-receptor blockers, Monounsaturated fatty acids, or Polyunsaturated fatty acids. FXR agonists (for example Obeticholic acid), LOXL2 antagonists (for example Simtuzumab), ASK1 antagonists (for example Selonsertib), PPAR agonists (for example clofibrate, gemfibrozil, ciprofibrate, bezafibrate, fenofibrate, thiazolidinediones, ibuprofen, GW-9662, aleglitazar, muraglitazar or tesaglitazar), Acetyl CoA-Carboxylase (ACC) antagonists (for example NDI-010976, PF-05221304), CCR2/CCR5 (for example Cenicriviroc), VAP1 antagonist.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif©), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of formula I and one or more additional therapeutic agents. The therapeutic agent may be administered together with a compound of formula I, or may be administered prior to or following administration of a compound of formula I. Suitable therapeutic agents are described in further detail below. In certain embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a compound of formula I and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating rheumatoid arthritis comprising administering to a patient in need thereof a compound of formula I and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a compound of formula I and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating cutaneous lupus erythematosus or systemic lupus erythematosus comprising administering to a patient in need thereof a compound of formula I and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating Crohn's disesase, ulcerative colitis, or inflammatory bowel disease comprising administering to a patient in need thereof a compound of formula I and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a compound of formula I and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a compound of formula I and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In another embodiment, the present invention provides a method of treating a hematological malignancy comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a solid tumor comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a hematological malignancy comprising administering to a patient in need thereof a compound of formula I and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a compound of formula I and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating or lessening the severity of a disease comprising administering to a patient in need thereof a compound of formula I and a BTK inhibitor, wherein the disease is selected from inflammatory bowel disease, arthritis, cutaneous lupus erythematosus, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, cutaneous lupus erythematosus, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a compound of formula I and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a compound of formula I and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments the present invention provides a method of treating or lessening the severity of a disease comprising administering to a patient in need thereof a compound of formula I and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

In some embodiments, the disease is an autoimmune disorder, an inflammatory disorder, a proliferative disorder, an endocrine disorder, a neurological disorder, or a disorder associated with transplantation. In some embodiments the JH2 binding compound is a compound of formula I. Other suitable JH2 domain binding compounds include those described in WO2014074660A1, WO2014074661A1, WO2015089143A1, the entirety of each of which is incorporated herein by reference. Suitable JH1 domain binding compounds include those described in WO2015131080A1, the entirety of which is incorporated herein by reference.

The compounds and compositions, according to the method of the present invention, may be administered using any amount and any route of administration effective for treating or lessening the severity of an autoimmune disorder, an inflammatory disorder, a proliferative disorder, an endocrine disorder, a neurological disorder, or a disorder associated with transplantation. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting OXER1 activity in a biological sample comprising the step of contacting said biological sample with a compound of this invention, or a composition comprising said compound.

According to another embodiment, the invention relates to a method of inhibiting OXER1, or a mutant thereof, activity in a biological sample comprising the step of contacting said biological sample with a compound of this invention, or a composition comprising said compound.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition of OXER1 (or a mutant thereof) activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of inhibiting OXER1 activity in a patient comprising the step of administering to said patient a compound of the present invention, or a composition comprising said compound.

According to another embodiment, the invention relates to a method of inhibiting activity of OXER1, or a mutant thereof, in a patient comprising the step of administering to said patient a compound of the present invention, or a composition comprising said compound. According to certain embodiments, the invention relates to a method of reversibly or irreversibly inhibiting one or more of OXER1, or a mutant thereof, activity in a patient comprising the step of administering to said patient a compound of the present invention, or a composition comprising said compound. In other embodiments, the present invention provides a method for treating a disorder mediated by OXER1, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C₁-1033, EKB-569, GW-2016, El.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S—001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ- tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R¹¹⁵⁷⁷⁷ (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, the present invention provides a method of treating AML associated with an ITD and/or D835Y mutation, comprising administering a compound of the present invention together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1 ,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™)

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden),V-11294A (Napp), BAY19-8004 (Bayer), SCH-351591 (Schering- Plough), Arofylline (Almirall Prodesfarma), PD189659/PD168787 (Parke-Davis), AWD-12-281 (Asta Medica), CDC-801 (Celgene), SeICID™CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fni4, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonist of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Ikena Oncology, formerly known as Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (OPDIVO®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (POMALYST®, Celgene); lenalidomide (REVLIMID®, Celgene); ingenol mebutate (PICATO®, LEO Pharma).

In some embodiments, an immuno-oncology agent is a cancer vaccine. In some embodiments, the cancer vaccine is selected from sipuleucel-T (PROVENGE®, Dendreon/Valeant Pharmaceuticals), which has been approved for treatment of asymptomatic, or minimally symptomatic metastatic castrate-resistant (hormone-refractory) prostate cancer; and talimogene laherparepvec (IMLYGIC®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (REOLYSIN®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-lh68/GLV-lh153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hlIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June et al.; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&μg=1].

In some embodiments, an immunostimulatory agent is an activator of retinoic acid receptor-related orphan receptor γ (RORyt). RORyt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCTO2124850) and ovarian cancer (NCT02431559).

Other immuno-oncology agents that can be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncoloby target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE®) antibody construct activates T cells. In some embodiments, a bispecific T cell engager (BITE®) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BITE®-activated T cells. In some embodiment, the bystander cells comprise tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the checkpoint inhibitor is a biologic therapeutic or a small molecule. In some embodiments, the checkpoint inhibitor is a monoclonal antibody, a humanized antibody, a fully human antibody, a fusion protein or a combination thereof. In some embodiments, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor is an immunostimulatory agent, a T cell growth factor, an interleukin, an antibody, a vaccine or a combination thereof. In some embodiments, the interleukin is IL-7 or IL-15. In some embodiments, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors can include small molecule inhibitors or can include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA®). In some embodiments, the checkpoint inhibitor is selected from nivolumab (anti-PD-1 antibody, OPDIVO®, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and I1MP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that can be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that can be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that can be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that can be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that can be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that can be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that can be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that can be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that can be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that can be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a OXER1 inhibitor. Implantable devices coated with a compound of this invention are another embodiment of the present invention.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein. Additional compounds of the invention were prepared by methods substantially similar to those described herein in the Examples and methods known to one skilled in the art.

General information: All evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (200-300 mesh). Solvent systems are reported as mixtures by volume. All NMR spectra were recorded on a Bruker 400 (400 MHz) spectrometer. ¹H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on an Agilent 1200 series 6110 or 6120 mass spectrometer with electrospray ionization and excepted as otherwise indicated,* the general LCMS condition was as follows: Waters X Bridge C18 column (50 mm×4.6 mm); Flow Rate: 2.0 mL/min, the column temperature: 40° C. Methods for HPLC analysis were one of the following:
Method 1: Agilent LCMS 1200-6110, Column: Waters X-Bridge C18 (50 mm *4.6 mm*3.5 m); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min.
Method 2: Agilent LCMS 1200-6120, Column: Waters X-Bridge C18 (50 mm *4.6 mm*3.5 m); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.
Method 3: Agilent HPLC 1200; Column: L-column2 ODS (150 mm *4.6 mm*5.0 m); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min.
Method 4: Agilent HPLC 1200, Column: Waters X-Bridge C18 (150 mm *4.6 mm*3.5 m); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min.

General procedure A (Wittig reaction to get the alkene intermediate): To a suspension of phosphorous ylide (1.0 eq.) in THF was added t-BuOK (3.0 eq.) at 0° C. The mixture was stirred for 30 min and aldehyde (1.0 eq.) in THF was added dropwise. The reaction mixture was allowed to warm to room temperature and stirred for 16 h. Saturated NH₄Cl solution was added at 0° C. and the mixture was acidified with HCl (1.0 M) to pH˜3, extracted with EtOAc, the combined organic layer was washed with brine, dried over anhydrous Na₂SO₄ and evaporated under reduced pressure. The crude was purified by silica gel chromatography to afford the alkene intermediate.

General procedure B (Hydrogenation from the alkene to the saturated alkane): To a stirred solution of alkene (1.0 eq.) in EtOAc was added 10% Pd/C (0.1 eq. w/w) under H2 atmosphere (1.0 atm). The reaction mixture was stirred at room temperature for 8 h until the reaction was complete (by LCMS) then filtered. The residue was washed with EtOAc, the combined filtrate was concentrated under reduced pressure to afford the saturated alkane intermediate.

General procedure C (Reduction from the carboxylic acid to the primary alcohol): To a stirred solution of the carboxylic acid (1.0 eq.) in THF was added LiAlH₄ (1.0 eq.) slowly at −20° C. The reaction mixture was allowed to warm to room temperature and stirred for 5 h until the reaction was complete (by LCMS). Water (2.0 eq.), NaOH (a.q. 15%, 2.0 eq.) and water (6.0 eq.) was added dropwise at 0° C. successively to quench the reaction. The mixture was filtered and the filtrate was dried over anhydrous Na₂SO₄ and evaporated under reduced pressure to afford the crude product alcohol, which was used to the next step directly.

General procedure D (Esterification of the alcohol derivatives to mesylate ester with Ms₂O): To a solution of the alcohol derivatives (1.0 eq.) and TEA (2.0 eq.) in THF was added Ms₂O (1.5 eq.) at 0° C. The mixture was allowed to warm to room temperature and stirred for another 2 h then quenched with water and extracted with DCM. The combined organic phase was washed with water and brine, dried over anhydrous Na₂SO₄ and concentrated to give the crude mesylate ester, which was used to the next step directly.

General procedure E (Bromo derivative formation from the mesylate ester): To a solution of the mesylate ester (1.0 eq.) in acetone was added LiBr (2.0 eq.) at room temperature. The mixture was stirred at refluxed overnight. Water was added and the mixture was extracted with DCM. The combined organic layer was dried over anhydrous Na₂SO₄, filtered and concentrated to give the bromo derivative, which was used to the next step directly.

General procedure F (Phosphorous ylide formation from the bromo derivatives): To a stirred solution of bromo derivatives (1.0 eq.) in acetonitrile was added PPh₃ (2.0 eq.). The reaction mixture was reflux at 80° C. overnight. The solvent was evaporated under reduced pressure and the crude was purified by silica gel chromatography (10% MeOH/DCM) to afford phosphorous ylide derivatives.

General procedure G (Acylation step): To a stirred solution of indole/azaindole derivatives (1.0 eq.) in dichloromethane was added Me₂AlCl (1.0 M in hexane, 2.0 eq.) at 0° C. After 45 min, acyl chloride (2.0 eq.) in CH₂Cl₂ (1 mL) was added dropwise at room temperature and the reaction mixture was stirred for another 2 h. The reaction was quenched by adding water and extracted with EtOAc. The organic layer was combined, washed with brine and dried over Na₂SO₄. The solvent was evaporated under reduced pressure and the crude was purified by silica gel chromatography (30% EtOAc/Hex) to afford the acylation product.

General procedure H (Hydrolysis from ester to the carboxylic acid): To a mixture of ester derivatives (1.0 eq.) in THF/H₂O (5/1, v/v) was added lithium hydroxide (5.0 eq.). The reaction mixture was stirred at room temperature for 16 h until the reaction was complete (by LCMS). Saturated NH₄Cl solution was added at 0° C. and the mixture was acidified with HCl (1.0 M) to pH ˜6.0 then extracted with EtOAc, the combined organic layer was washed with brine, dried over anhydrous Na₂SO₄ and evaporated under reduced pressure. The crude was purified by Prep-HPLC to afford the desired product.

General procedure I (Ether formation): To a stirred solution of alcohol (1.0 eq.) in DMF was added NaH (⁶0% in mineral oil, 1.0 eq.). The reaction mixture was stirred at 0° C. for 0.5 h under N₂, then bromo derivatives (1.0 eq.) was added slowly. The reaction mixture was stirred at room temperature for 2 hours until the reaction was complete. The suspension was diluted with NaCl (aq.), extracted with EtOAc. The organic layer was washed with brine, dried over anhydrous Na₂SO₄, filtered, concentrated in vacuo and purified by column chromatography to afford the ether derivatives.

General procedure J (Oxidation from alcohol to aldehyde): To a solution of alcohol (1.0 eq.) in DCM was added DMP (1.2 eq.) at 0° C. The mixture was stirred for 30 min at 0° C. Saturated NaHCO₃ (aq.) was added to the mixture and it was extracted with DCM. The combined organic layer was washed with brine, dried over anhydrous Na₂SO₄, filtered and concentrated in vacuo. The crude was purified by silica gel chromatography to afford aldehyde product.

General procedure K (Grignard reagent addition on the aldehyde): To a solution of aldehyde (1.0 eq.) in THF was added Grignard reagent (1.2 eq.) at 0° C. The mixture was stirred at room temperature for 1 h. Water was added and the mixture was extracted with EtOAc. The combined organic layer was dried over anhydrous Na₂SO₄, filtered and concentrated in vacuo. The crude was purified by silica gel chromatography to afford the corresponding secondary alcohol product.

General procedure L (Alkylation on the terminal alkyne): To a suspension of terminal alkyne (1.0 eq.) in THF was added n-BuLi (1.2 eq.) at −78° C. The mixture was stirred for 1 hour and bromo derivative (1.0 eq.) in HMPA was added dropwise. The reaction mixture was allowed to warm to rt and stirred for another 16 h. Saturated NH₄Cl solution was added and the reaction mixture was extracted with EtOAc. The combined organic layer was washed with brine, dried over anhydrous Na₂SO₄ and the solvent was evaporated under reduced pressure. The crude was purified by silica gel chromatography to afford the desired alkylation product.

General procedure M (Sonogashira Coupling): To a stirred solution of aryl halide (1.0 eq.) in THF was added terminal alkynes (1.1 eq.), Pd(PPh₃)₂Cl₂ (0.1 eq.), CuI (0.1 eq.) and TEA (3.0 eq.). The reaction mixture was stirred at 60° C. for 16 h under nitrogen. The mixture was filtered through a pad of celite then water and EA were added. The organic phases was collected and evaporated under reduced pressure and purified by silica gel chromatography to afford the coupling product.

General procedure N (Cyclization to give the indole/azaindole derivatives): To a stirred solution of alkyne (1.0 eq.) in DMF was added t-BuOK (1.4 eq.). The reaction mixture was stirred at 80° C. for 0.5 h under nitrogen. The mixture was filtered through a pad of diatomite and purified by prep-HPLC to give the indole/azaindole derivatives.

### Example 1: Synthesis of Compound I-24

### Synthetic Scheme for Compound I-24

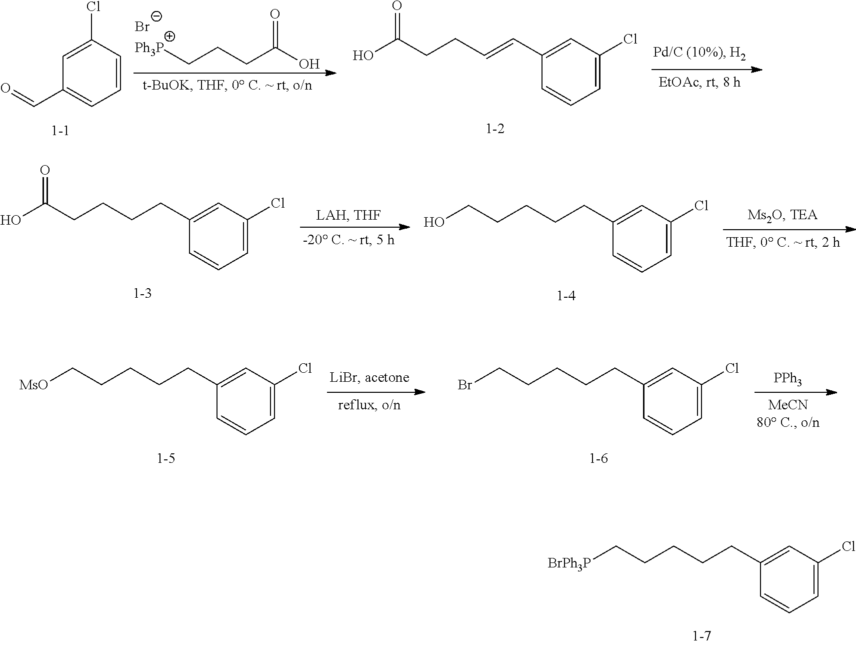

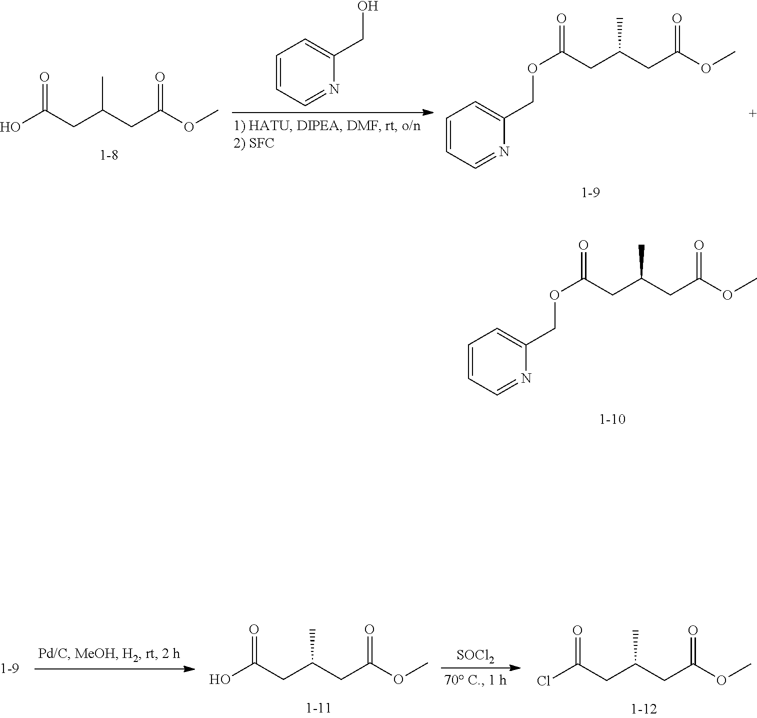

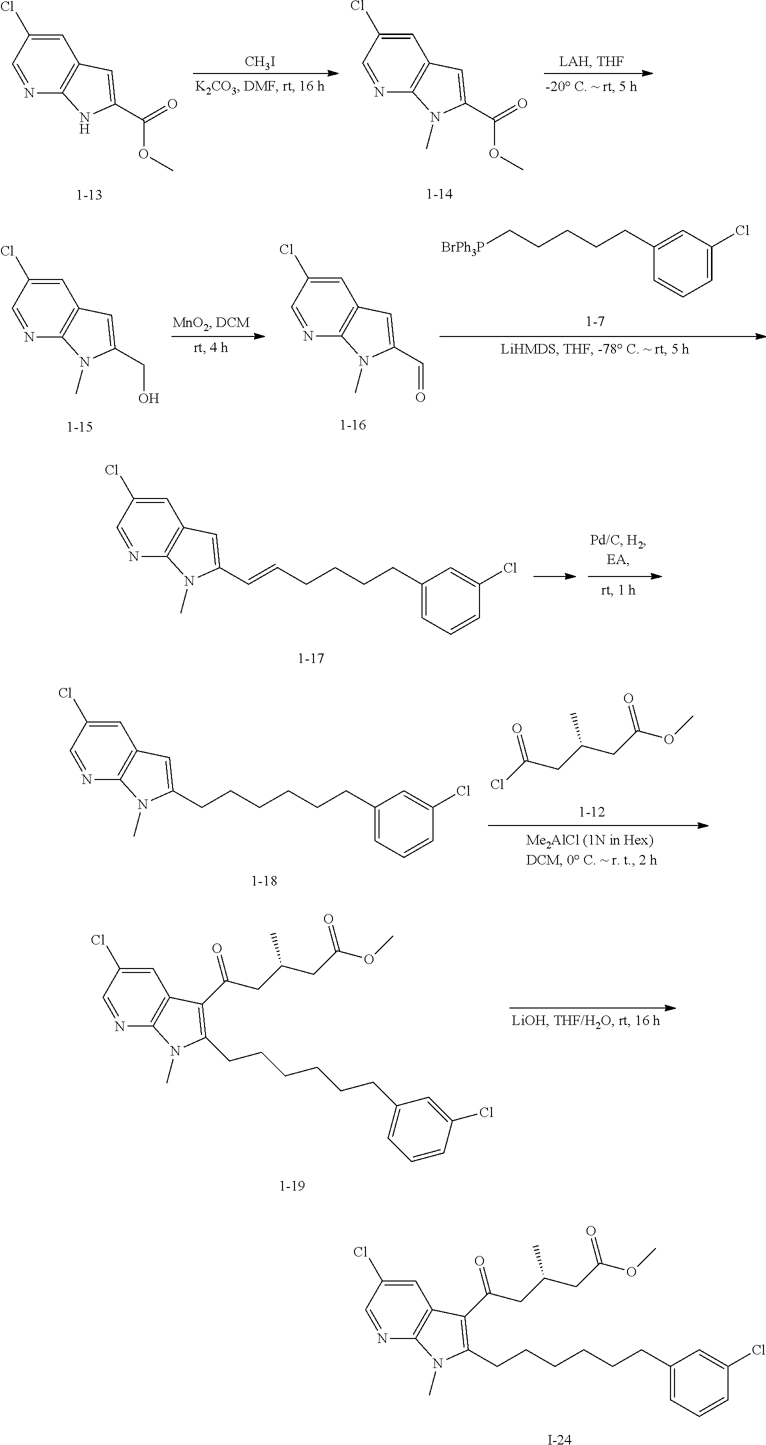

### 1.1 The Synthesis of Intermediate 1-2

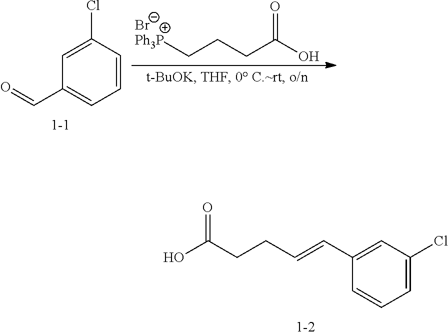

To a suspension of (3-carboxypropyl)triphenylphosphonium bromide (207.6 g, 483.7 mmol) in THF (1600 mL) was added t-BuOK (135.7 g, 1.21 mol) at 0° C. The mixture was stirred for 30 min and 1-1 (68 g, 483.7 mmol) in THF (200 ml) was added dropwise. The reaction mixture was allowed to warm to room temperature and stirred for 16 h. Saturated NH₄Cl solution was added at 0° C. and the mixture was acidified with HCl (1.0 M) to pH˜3, extracted with EtOAc, the combined organic layer was washed with brine, dried over anhydrous Na₂SO₄ and evaporated under reduced pressure. The crude was purified by silica gel chromatography (20% EtOAc/Hexane) to afford 1-2 (71.0 g, 70%) as colorless oil. LC-MS m/z: 211.2 [M+H]⁺.

### 1.2 The Synthesis of Intermediate 1-3

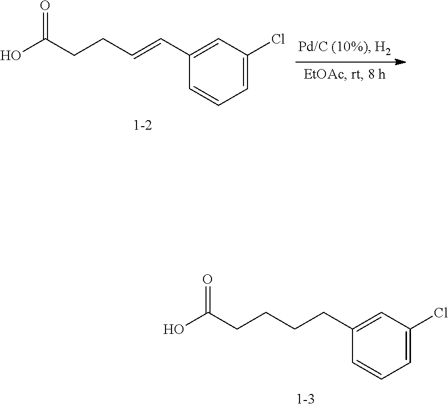 [SMILES: O=C(O)CC/C=C/c1cccc(Cl)c1 ; O=C(O)CCCCc1cccc(Cl)c1]

To a stirred solution of 1-2 (71.0 g, 338.1 mmol) in EtOAc (500 mL) was added 10% Pd/C (8.0 g) under H₂ atmosphere (1.0 atm). The reaction mixture was stirred at room temperature for 8 h until the reaction was complete (by LCMS) and then filtered. The residue was washed with EtOAc, and the combined filtrate was concentrated under reduced pressure to afford 1-3 (71 g, 99%) as colorless oil. LC-MS m/z: 213.1 [M+H]⁺.

### 1.3 The Synthesis of Intermediate 1-4

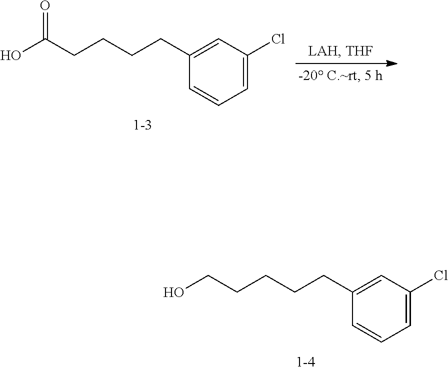 [SMILES: O=C(O)CCCCc1cccc(Cl)c1 ; OCCCCCc1cccc(Cl)c1]

To a stirred solution of 1-3 (71 g, 334.9 mmol) in THF (500 mL) was added LiAlH₄ (12.7 g, 334.9 mmol) slowly at −20° C. The reaction mixture was allowed to warm to room temperature and stirred for 5 h until the reaction was complete (by LCMS). Water (13 mL), NaOH (a.q. 15%, 13 mL) and water (39 mL) was added dropwise at 0° C. successively to quench the reaction. The mixture was filtered and the filtrate was dried over anhydrous Na₂SO₄ and evaporated under reduced pressure to afford the crude product 1-4 (60.6 g, 91%) as brown oil, which was used to the next step directly. LC-MS m/z: 199.9 [M+H]⁺.

### 1.4 The Synthesis of Intermediate 1-5

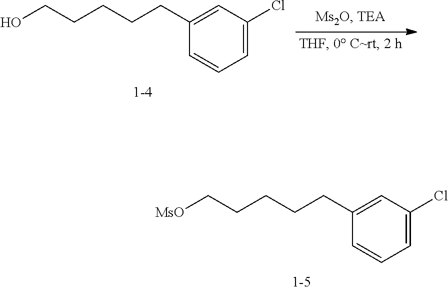 [SMILES: OCCCCCc1cccc(Cl)c1 ; CS(=O)(=O)OCCCCCc1cccc(Cl)c1]

To a solution of 1-4 (60.6 g, 305 mmol) and TEA (61.6 g, 610 mmol) in THF (800 mL) was added methanesulfonic anhydride (63.6 g, 366 mmol) under Ar atmosphere at 0° C. Then the mixture was allowed to warm to room temperature and stirred for another 2 h. Saturated NaHCO₃ (aq., 80 mL) was added to the mixture and it was extracted with DCM (300 mL×3). The combined organic layer was washed with brine (200 mL×2), dried over anhydrous Na₂SO₄, filtered and concentrated in vacuo to give 1-5 (84.0 g, 91%) as yellow oil. LC-MS m/z: 277.2 [M+H]⁺.

### 1.5 The Synthesis of Intermediate 1-6

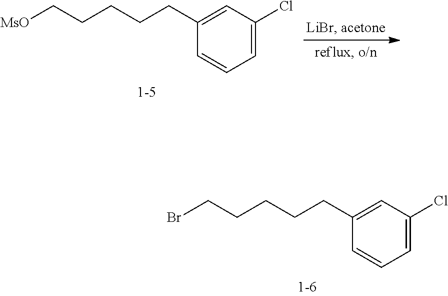 [SMILES: CS(=O)(=O)OCCCCCc1cccc(Cl)c1 ; Clc1cccc(CCCCCBr)c1]

To a solution of 1-5 (84.0 g, 303.5 mmol) in acetone (800 mL) was added LiBr (52.7 g, 606.9 mmol) at room temperature. The mixture was stirred at refluxed overnight. Water (300 mL) was added and the mixture was extracted with DCM (400 mL×3). The combined organic layer was dried over anhydrous Na₂SO₄, filtered and concentrated in vacuo to give 1-6 (75.4 g, yield: 95%) as yellow oil. LC-MS m/z: 263.1 [M+H]⁺.

### 1.6 The Synthesis of Intermediate 1-7

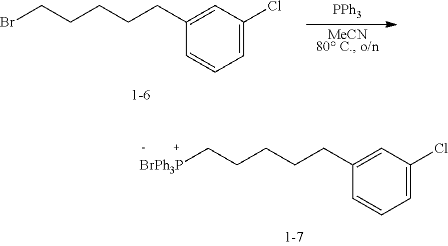

To a stirred solution of 1-6 (75.4 g, 288.2 mmol) in acetonitrile (600 mL) was added PPh₃ (151.0 g, 576.4 mmol). The reaction mixture was reflux at 80° C. overnight. The solvent was evaporated under reduced pressure and the crude was purified by silica gel chromatography (10% MeOH/DCM) to afford 1-7 (115.0 g, 76%) as a white solid. LC-MS m/z: 423.1 [M+H]⁺.

### 1.7 The Synthesis of Intermediate 1-9

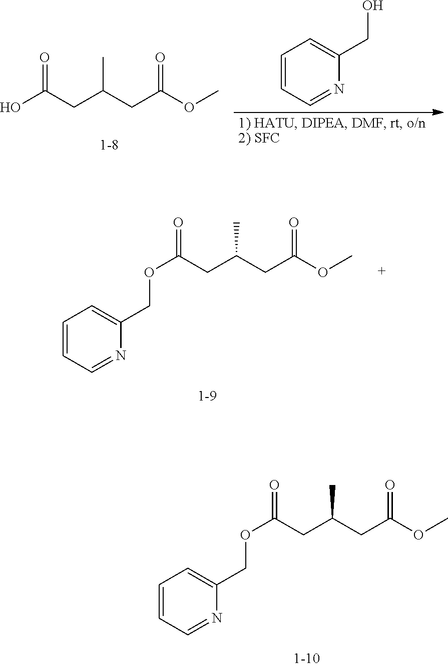

To a stirred solution of 1-8 (50.0 g, 312.5 mmol) in DMF (600 mL) was added HATU (148.4 g, 390.6 mmol) and DIEA (155 mL, 937.5 mmol) at an ice bath. After stirring at room temperature for 1 h, pyridine-2-ylmethanol (40.88 g, 375.0 mmol) was added and the reaction mixture was stirred overnight. When the reaction finished (by LCMS), the reaction mixture was quenched with water (1.0 L), extracted with EtOAc (600 mL×3), washed with water and brine, and removed the solvent under reduced pressure. The crude was purified by silica gel chromatography (10-33% EtOAc in PE), and prep-HPLC. Enanatiomers 1-9 and 1-10 were separated by chiral-HPLC under super-critical fluid conditions (Column: CHIRALPAK AY-3 (4.6 mm×100 mm), Solvent: 15% EtOH in liquid CO₂ (at 35° C. and 2000 psi), Flow rate: 2 mL/min., Detector: 215 nm), to get 1-9 (t_{R}: 1.542 min., 18 g, yield: 23%) as yellow oil. t_{R} of 1-10: 1.895 mins.

### 1.8 The Synthesis of Intermediate 1-11

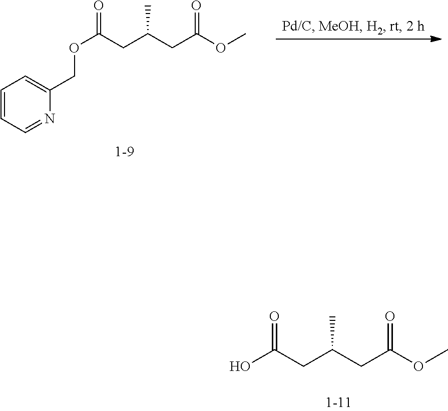 [SMILES: COC(=O)C[C@@H](C)CC(=O)OCc1ccccn1 ; "Pd/C, MeOH, H2, rt, 2 h" ; COC(=O)C[C@@H](C)CC(=O)O]

To a stirred solution of 1-9 (18.0 g, 71.6 mmol) in MeOH (200 mL) was added 15% Pd/C (2.7 g) under H₂ atmosphere (1.0 atm). The reaction mixture was stirred at room temperature for 2 h until the reaction was complete (by LCMS) then filtered. The residue was washed with EtOAc and the combined filtrate was concentrated under reduced pressure. The crude was purified by silica gel chromatography (24% EtOAc/0.1% formic acid in PE) to get 1-11 (8.5 g, yield: 74%) as light yellow oil. LC-MS m/z: 161.2 [M+H]⁺.

### 1.9 The Synthesis of Intermediate 1-12

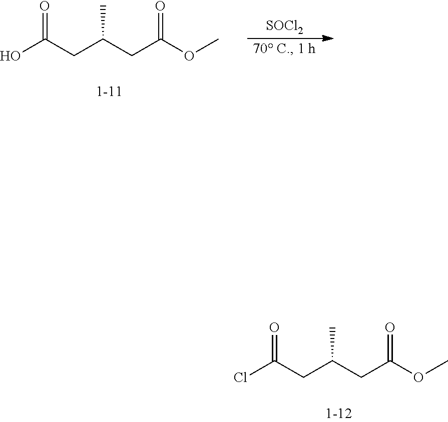

A solution of 1-11 (200 mg, 1.25 mmol) in SOCl₂ (1.5 mL) was stirred at 70° C. for 1 h. The residue was concentrated under reduced pressure to afford the crude, the crude was co-distilled with DCM (2×10 mL) to afford the product 1-12 (190 mg, yield: 85%) as yellow oil, which was used to the next step directly.

### 1.10 The Synthesis of Intermediate 1-14

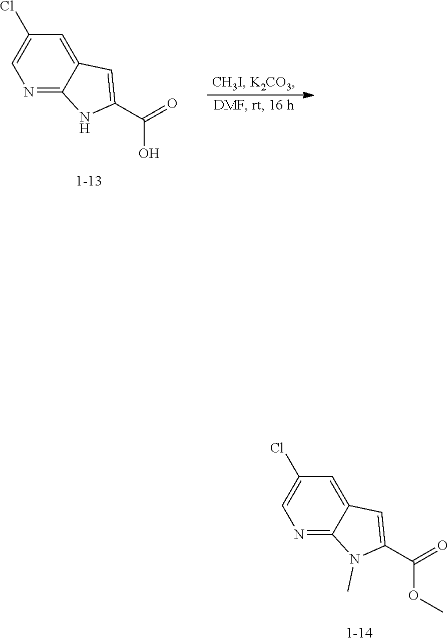

To a solution of 1-13 (5.0 g, 25.4 mmol) and K₂CO₃ (10.5 g, 76.3 mmol) in DMF (30 mL) was added methyl iodide (7.2 g, 50.9 mmol) under Ar atmosphere. Then the mixture was stirred at room temperature overnight until the reaction was complete (by LCMS). The reaction mixture was poured slowly into cool water and the precipitate was collected and dried to afford 1-14 (5.0 g, 88%) as a brown solid. ¹H NMR (400 MHz, CDCl₃) δ 8.41 (d, J=2.0 Hz, 1H), 7.96 (d, J=2.0 Hz, 1H), 7.17 (s, 1H), 4.15 (s, 3H), 3.95 (s, 3H).

### 1.11 The Synthesis of Intermediate 1-15

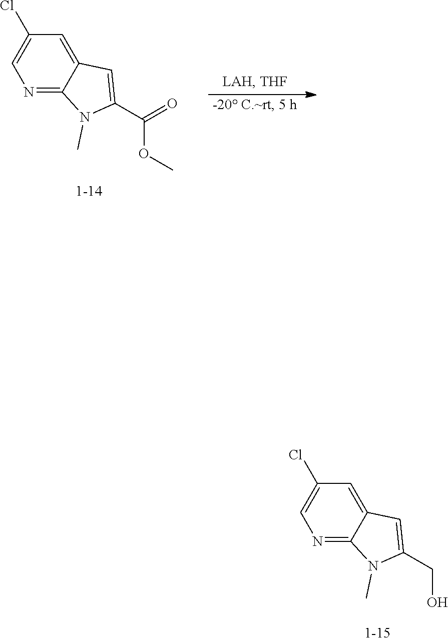

To a stirred solution of 1-14 (5.0 g, 22.3 mmol) in THF (50 mL) was added LiAlH₄ (848 mg, 22.3 mmol) slowly at −20° C. The reaction mixture was allowed to warm to room temperature and stirred for 5 h until the reaction was complete (by LCMS). Water (0.9 mL), NaOH (a.q. 15%, 2.7 mL) and water (0.9 mL) was added dropwise at 0° C. successively to quench the reaction. The mixture was filtered and the filtrate was dried over anhydrous Na₂SO₄ and evaporated under reduced pressure to afford the crude product 1-15 (3.1 g, 71%) as a grey solid, which was used to the next step directly.

### 1.12 The Synthesis of Intermediate 1-16

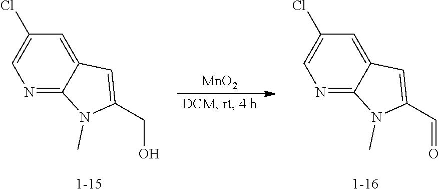

To a mixture of 1-15 (3.1 g, 15.8 mmol) in DCM (25 mL) was added MnO₂ (31.0 g, 356 mmol). The reaction mixture was stirred at room temperature for 4 h until the reaction was complete (by LCMS) then filtered. The residue was washed with EtOAc and the combined filtrate was concentrated under reduced pressure to afford 1-16 (2.5 g, 80%) as a grey solid. 1.13 The Synthesis of Intermediate 1-17

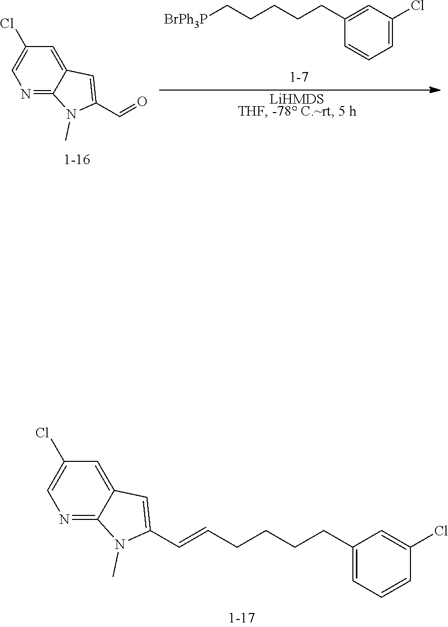

To a suspension of 1-16 (2.5 g, 12.6 mmol) in THF (100 mL) was added LiHMDS (1.0 M in THF, 31.6 mL, 31.6 mmol) at −78° C. The mixture was stirred for 30 min then 1-7 (9.7 g, 50.3 mmol) in THF (100 ml) was added dropwise. The reaction mixture was allowed to warm to room temperature and stirred for 5 h. Saturated NH₄Cl solution was added and the reaction mixture was extracted with EtOAc. The combined organic layer was washed with brine, dried over anhydrous Na₂SO₄ and the solvent was evaporated under reduced pressure. The crude was purified by silica gel chromatography (5% EtOAc/Hexane) to afford 1-17 (2.9 g, 64%) as a light-yellow solid.

### 1.14 The Synthesis of Intermediate 1-18

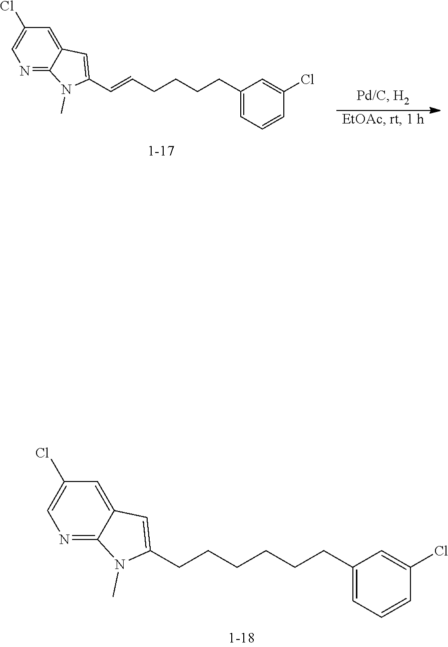 [SMILES: Cn1c(/C=C/CCCCc2cccc(Cl)c2)cc2cc(Cl)cnc21 ; "EtOAc, rt, 1 h" ; "Pd/C, H2" ; Cn1c(CCCCCCc2cccc(Cl)c2)cc2cc(Cl)cnc21]

To a stirred solution of 1-17 (2.9 g, 8.1 mmol) in EtOAc (30 mL) was added 10% Pd/C (580 mg) under H₂ atmosphere (1.0 atm). The reaction mixture was stirred at room temperature for 1 h until the reaction was complete (by LCMS) then filtered. The residue was washed with EtOAc and the combined filtrate was concentrated under reduced pressure to afford 1-18 (2.3 g, 79%) as a light-yellow solid.

### 1.15 The Synthesis of Intermediate 1-19

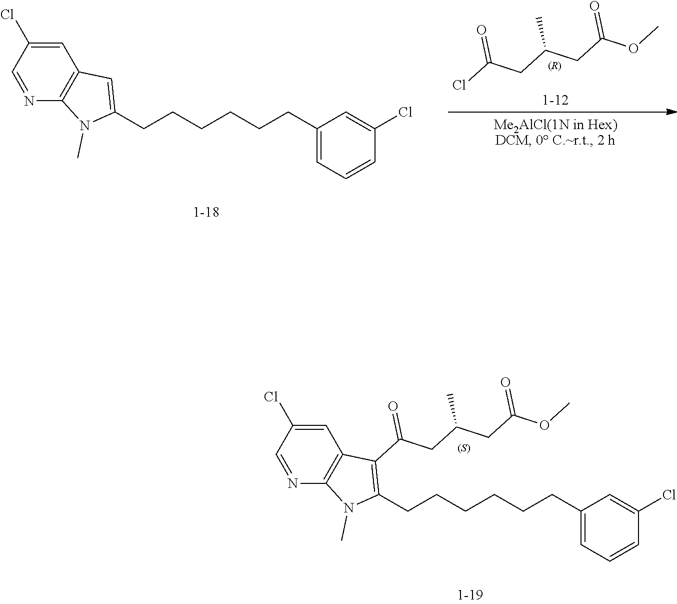

To a stirred solution of 1-18 (360 mg, 1.0 mmol) in dichloromethane (15 mL) was added Me₂AlCl (1.0 M in hexane, 1.0 mL, 1.0 mmol) at 0° C. After 45 min, 1-12 (1.4 g, 6.4 mmol) in CH₂Cl₂ (1 mL) was added dropwise at room temperature and the reaction mixture was stirred for another 2 h. The reaction was quenched by adding water and extracted with EtOAc. The organic layer was combined, washed with brine and dried over Na₂SO₄. The solvent was evaporated under reduced pressure and the crude was purified by silica gel chromatography (30% EtOAc/Hex) to afford 1-19 (320 mg, 64%) as yellow oil.

### 1.16 The Synthesis of Compound I-24

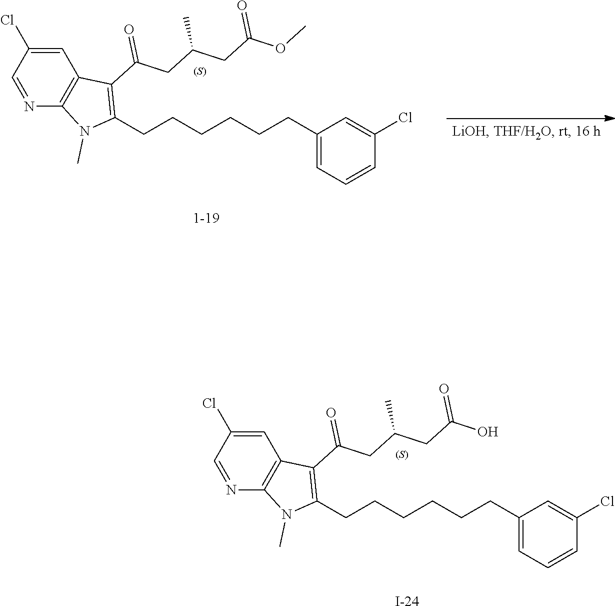

To a mixture of 1-19 (320 mg, 0.64 mmol) in THF/H₂O (10 mL/2 mL) was added lithium hydroxide (120 mg, 3.2 mmol), the reaction mixture was stirred at room temperature for 16 h until the reaction was complete (by LCMS). Saturated NH₄Cl solution was added at 0° C. and the mixture was acidified with HCl (1.0 M) to pH ˜6.0 then extracted with EtOAc, the combined organic layer was washed with brine, dried over anhydrous Na₂SO₄ and evaporated under reduced pressure. The crude was purified by Prep-HPLC to afford 1-24 (130 mg, 42%) as a white solid. ¹H NMR (400 MHz, DMSO-d₆) δ 12.18 (br, 1H), 8.37 (d, J=2.0 Hz, 1H), 8.32 (d, J=2.0 Hz, 1H), 7.31-7.26 (m, 2H), 7.22 (d, J=7.6 Hz, 1H), 7.16 (d, J=7.6 Hz, 1H), 3.80 (s, 3H), 3.18 (t, J=7.6 Hz, 2H), 3.04 (dd, J=16.4, 5.6 Hz, 1H), 2.86 (dd, J=16.8, 7.6 Hz, 1H), 2.58 (t, J=7.6 Hz, 2H), 2.41 (d, J=5.6 Hz, 1H), 2.37 (d, J=5.6 Hz, 1H), 2.17 (dd, J=15.6, 8.0 Hz, 1H), 1.60-1.56 (m, 4H), 1.46-1.41 (m, 2H), 1.38-1.34 (m, 2H), 0.96 (d, J=6.8 Hz, 3H).

LC-MS m/z: 489.0 [M+H]⁺.

Additional exemplary compounds were prepared following methods substantially similar to the method described above and herein. Data for these compounds are provided in Table 2 and 2a below.

TABLE 2
Characterization Data for Additional Exemplary Compounds

| Compound | | | |
|---|---|---|---|
| No. | Chemical Structure | M + 1 | ¹H NMR (400 MHz) |
| I-1 | 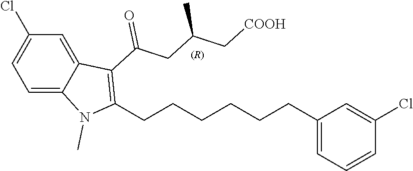 | 488.0 | ¹H NMR (400 MHz, DMSO-d₆) δ 12.03 (s, 1H), 7.96 (d, J = 2.0 Hz, 1H), 7.59 (d, J = 8.8 Hz, 1H), 7.31-7.21 (m, 4H), 7.16 (d, J = 7.6 Hz, 1H), 3.75 (s, 3H), 3.16-3.09 (m, 2H), 2.98 (dd, J = 16.8, 6.0 Hz, 1H), 2.84 (dd, J = 16.8, 7.6 Hz, 1H), 2.61-2.54 (m, 2H), 2.48-2.43 (m, 1H), 2.39 (dd, J = 16.0, 5.6 Hz, 1H), 2.17 (dd, J = 15.6, 8.0 Hz, 1H), 1.62-1.52 (m, 4H), 1.45-1.40 (m, 2H), 1.35- 1.32 (m, 2H), 0.97 (d, J = 6.4 Hz, 3H). |
| I-2 | 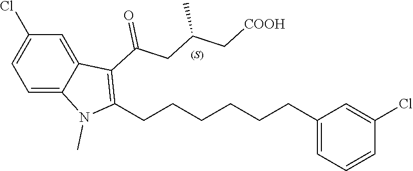 | 488.0 | ¹H NMR (400 MHz, DMSO-d₆) δ 12.07 (s, 1H), 7.96 (d, J = 1.6 Hz, 1H), 7.59 (d, J = 8.8 Hz, 1H), 7.31-7.21 (m, 4H), 7.16 (d, J = 7.6 Hz, 1H), 3.75 (s, 3H), 3.13 (t, J = 7.6 Hz, 2H), 2.98 (dd, J = 16.8, 5.6 Hz, 1H), 2.84 (dd, J = 16.4, 7.2 Hz, 1H), 2.56-2.60 (m, 2H), 2.43-2.48 (m, 1H), 2.39 (dd, J = 15.6, 5.6 Hz, 1H), 2.17 (dd, J = 15.6, 8.4 Hz, 1H), 1.52-1.60 (m, 4H), 1.40-1.46 (m, 2H), 1.32-1.37 (m, 2H), 0.97 (d, J = 6.8 Hz, 3H). |
| I-7 | 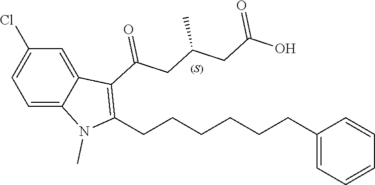 | 454.0 | ¹H NMR (400 MHz, DMSO-d₆) δ 12.13 (s, 1H), 7.96 (d, J = 2.0 Hz, 1H), 7.58 (d, J = 8.8 Hz, 1H), 7.28-7.23 (m, 3H), 7.19-7.15 (m, 3H), 3.74 (s, 3H), 3.13 (t, J = 7.6 Hz, 2H), 2.97 (dd, J = 16.4, 6.0 Hz, 1H), 2.86 (dd, J = 16.4, 7.6 Hz, 1H), 2.57 (t, J = 7.6 Hz, 2H), 2.52-2.50 (m, 1H), 2.39 (dd, J = 15.2, 5.6 Hz, 1H), 2.17 (dd, J = 15.6, 8.0 Hz, 1H), 1.62-1.52 (m, H), 1.48-1.40 (m, 2H), 1.38- 1.33 (m, 2H), 0.97 (d, J = 6.4 Hz, 3H). |
| I-8 | 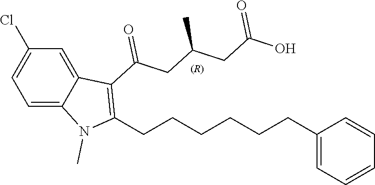 | 454.0 | ¹H NMR (400 MHz, DMSO-d₆) δ 12.10 (s, 1H), 7.96 (d, J = 1.6 Hz, 1H), 7.58 (d, J = 8.8 Hz, 1H), 7.28-7.24 (m, 3H), 7.19-7.13 (m, 3H), 3.75 (s, 3H), 3.13 (t, J = 7.6 Hz, 2H), 2.97 (dd, J = 16.4, 5.6 Hz, 1H), 2.86 (dd, J = 16.0, 7.6 Hz, 1H), 2.57 (t, J = 7.6 Hz, 2H), 2.52-2.50 (m, 1H), 2.37 (dd, J = 15.6 Hz, 5.6 Hz, 1H), 2.18 (dd, J = 15.2, 8.0 Hz, 1H), 1.62-1.54 (m, 4H), 1.48-1.40 (m, 2H), 1.38-1.35 (m, 2H), 0.97 (d, J = 6.8 Hz, 3H). |
| I-9 | 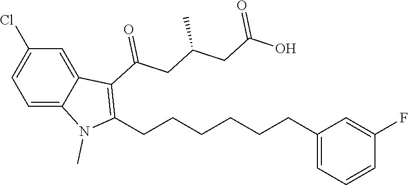 [SMILES: C[C@H](CC(=O)O)CC(=O)c1c(CCCCCCc2cccc(F)c2)n(C)c2ccc(Cl)cc12] | 471.9 | ¹H NMR (400 MHz, DMSO-d₆) δ 12.07 (s, 1H), 7.96 (d, J = 2.0 Hz, 1H), 7.58 (d, J = 8.8 Hz, 1H), 7.32-7.27 (m, 1H), 7.24 (dd, J = 8.8, 2.0 Hz, 1H), 7.04-6.95 (m, 3H), 3.75 (s, 3H), 3.13 (t, J = 7.6 Hz, 2H), 2.99 (dd, J = 16.4, 6.0 Hz, 1H), 2.84 (dd, J = 16.4, 8.0 Hz, 1H), 2.59 (t, J = 8.0 Hz, 2H), 2.51-2.50 (m, 1H), 2.38 (dd, J = 15.2, 5.6 Hz, 1H), 2.17 (dd, J = 15.2, 8.0 Hz, 1H), 1.61-1.52 (m, 4H), 1.46-1.40 (m, 2H), 1.38-1.32 (m, 2H), 0.97 (d, J = 6.4 Hz, 3H). |
| I-10 | 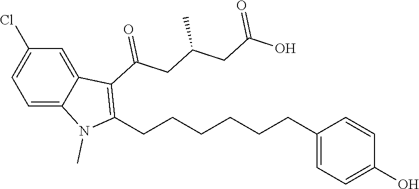 [SMILES: C[C@H](CC(=O)O)CC(=O)c1c(CCCCCCc2ccc(O)cc2)n(C)c2ccc(Cl)cc12] | 470.0 | ¹H NMR (400 MHz, DMSO-d₆) δ 12.09 (s, 1H), 9.10 (s, 1H), 7.96 (d, J = 2.0 Hz, 1H), 7.58 (d, J = 8.8 Hz, 1H), 7.24 (dd, J = 8.8, 2.0 Hz, 1H), 6.96 (d, J = 8.4 Hz, 2H), 6.65 (d, J = 8.8 Hz, 2H), 3.74 (s, 3H), 3.12 (t, J = 8.0 Hz, 2H), 2.98 (dd, J = 16.4, 5.6 Hz, 1H), 2.84 (dd, J = 16.4, 7.6 Hz, 1H), 2.56- 2.54 (m, 1H), 2.46-2.37 (m, 3H), 2.18 (dd, J = 15.6, 8.0 Hz, 1H), 1.59-1.26 (m, 8H), 0.98 (d, J = 6.4 Hz, 3H). |
| I-11 | 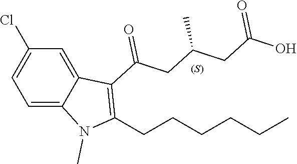 | 378.0 | ¹H NMR (400 MHz, DMSO-d₆) δ 12.00 (brs., 1H), 7.96 (d, J = 1.6 Hz, 1H), 7.59 (d, J = 8.8 Hz, 1H), 7.24 (dd, J = 8.8, 1.6 Hz, 1H), 3.76 (s, 3H), 3.14 (t, J = 8.0 Hz, 2H), 2.96 (dd, J = 16.4, 6.0 Hz, 1H), 2.85 (dd, J = 16.4, 7.6 Hz, 1H), 2.43-2.32 (m, 2H), 2.17 (dd, J = 15.6, 8.0 Hz, 1H), 1.58-1.52 (m, 2H), 1.43-1.38 (m, 2H), 1.31-1.29 (m, 4H), 0.97 (d, J = 6.4 Hz, 3H), 0.88-0.85 (t, J = 6.4 Hz, 3H). |
| I-12 | 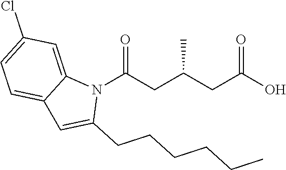 [SMILES: CCCCCCc1cc2ccc(Cl)cc2n1C(=O)C[C@H](C)CC(=O)O] | 364.0 | ¹H NMR (400 MHz, DMSO-d₆) δ 7.95 (s, 1H), 7.49 (d, J = 8.4 Hz, 1H), 7.21 (dd, J = 8.0, 1.2 Hz, 1H), 6.52 (s, 1H), 3.14 (dd, J = 16.4, 5.6 Hz, 2H), 3.00 (d, J = 8.0 Hz, 1H), 2.94-2.91 (m, 2H), 2.40-2.35 (m, 2H), 2.18 (dd, J = 15.2, 7.6 Hz, 1H), 1.99-1.93 (m, 1H), 1.66-1.58 (m, 2H), 1.36-1.31 (m, 3H), 1.27-1.26 (m, 4H), 0.97 (d, J = 6.8 Hz, 3H). |
| I-17 | 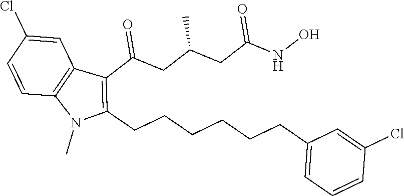 [SMILES: C[C@H](CC(=O)NO)CC(=O)c1c(CCCCCCc2cccc(Cl)c2)n(C)c2ccc(Cl)cc12] | 502.9 | ¹H NMR (400 MHz, DMSO-d₆) δ 10.41 (s, 1H), 8.72 (s, 1H), 7.94 (d, J = 2.0 Hz, 1H), 7.59 (d, J = 8.8 Hz, 1H), 7.31-7.21 (m, 4H), 7.16 (d, J = 7.2 Hz, 1H), 3.75 (s, 3H), 3.13 (t, J = 8.0 Hz, 2H), 2.97 (dd, J = 16.4, 5.2 Hz, 1H), 2.80 (dd, J = 16.4, 8.0 Hz, 1H), 2.58 (t, J = 7.6 Hz 2H), 2.53-2.51 (m, 1H), 2.09 (dd, J = 14.0, 6.0 Hz, 1H), 1.93 (dd, J = 14.0 Hz, 8.0 Hz, 1H), 1.62-1.52 (m, 4H), 1.48-1.40 (m, 2H), 1.37-1.34 (m, 2H), 0.92 (d, J = 6.4 Hz, 3H). |
| I-19 | 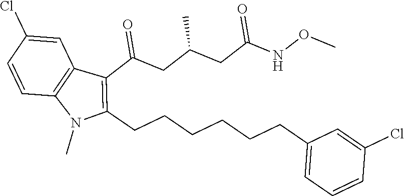 [SMILES: CONC(=O)C[C@@H](C)CC(=O)c1c(CCCCCCc2cccc(Cl)c2)n(C)c2ccc(Cl)cc12] | 517.0 | ¹H NMR (400 MHz, DMSO-d₆) δ 11.02 (s, 1H), 7.94 (d, J = 1.6 Hz, 1H), 7.59 (d, J = 8.8 Hz, 1H), 7.31-7.21 (m, 4H), 7.16 (d, J = 7.2 Hz, 1H), 3.75 (s, 3H), 3.58 (s, 3H), 3.13 (t, J = 8.0 Hz, 1H), 2.97 (dd, J = 16.4, 5.6 Hz, 1H), 2.80 (dd, J = 16.4, 8.0 Hz, 1H), 2.58 (t, J = 7.6 Hz, 2H), 2.53-2.51 (m, 1H), 2.11-2.06 (m, 1H), 1.92 (dd, J = 14.0 Hz, 8.0 Hz, 1H), 1.62-1.52 (m, 4H), 1.47-1.40 (m, 2H), 1.32-1.37 (m, 2H), 0.93 (d, J = 6.8 Hz, 3H). |
| I-39 | 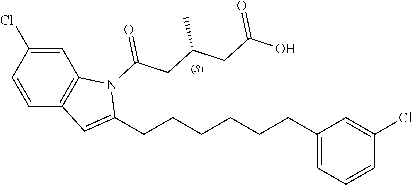 | 474.2 | ¹H NMR (400 MHz, DMSO-d₆) δ12.04 (s, 1H), 7.99 (s, 1H), 7.52 (d, J = 8.4 Hz, 1H), 7.40-7.22 (m, 4H), 7.16 (d, J = 7.6 Hz, 1H), 6.55 (s, 1H), 3.16 (dd, J = 16.4, 5.6 Hz, 1H), 3.04-2.93 (m, 3H), 2.58 (t, J = 7.6 Hz, 2H), 2.42 (dd, J = 15.6, 5.2 Hz, 1H), 2.25- 2.16 (m, 2H), 1.69-1.54 (m, 4H), 1.43-1.32 (m, 4H), 1.00 (d, J = 6.4 Hz, 3H). |
| I-40 | 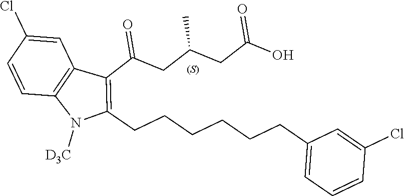 | 490.9 | ¹H NMR (400 MHz, DMSO-d₆) δ 12.00 (s, 1H), 7.96 (d, J = 2.0 Hz, 1H), 7.59 (d, J = 8.8 Hz, 1H), 7.31-7.21 (m, 4H), 7.16 (d, J = 7.6 Hz, 1H), 3.32 (s, 1H), 3.13 (d, J = 7.6 Hz, 1H), 2.99 (dd, J = 16.0, 5.6 Hz, 1H), 2.84 (dd, J = 16.4, 7.6 Hz, 1H), 2.58 (t, J = 7.6 Hz, 2H), 2.39 (dd, J = 15.6, 6.0 Hz, 1H), 2.17 (dd, J = 15.6, 8.0 Hz, 1H), 1.62-1.52 (m, 4H), 1.47-1.40 (m, 2H), 1.37-1.34 (m, 2H), 0.97 (d, J = 6.4 Hz, 3H). |
| I-48 | 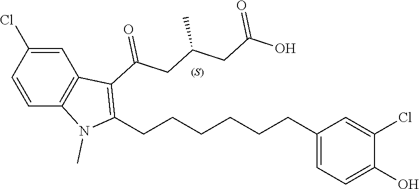 | 503.9 | ¹H NMR (400 MHz, DMSO-d₆) δ 12.06 (s, 1H), 9.85 (s, 1H), 7.96 (d, J = 1.6 Hz, 1H), 7.58 (d, J = 8.8 Hz, 1H), 7.24 (dd, J = 8.8, 2.0 Hz, 1H), 7.12 (d, J = 2.0 Hz, 1H), 6.94 (dd, J = 8.4, 2.0 Hz, 1H), 6.85 (d, J = 8.4 Hz, 1H), 3.75 (s, 3H), 3.13 (t, J = 8.0 Hz, 2H), 2.99 (dd, J = 16.4, 5.6 Hz, 1H), 2.84 (dd, J = 16.4, 7.6 Hz, 1H), 2.55-2.53 (m, 1H), 2.46 (t, J = 7.6 Hz, 2H), 2.39 (dd, J = 15.6, 6.0 Hz, 1H), 2.17 (dd, J = 15.2, 8.0 Hz, 1H), 1.59-1.49 (m, 4H), 1.46-1.39 (m, 2H), 1.35-1.32 (m, 2H), 0.97 (d, J = 6.8 Hz, |
| | | | 3H). |
| I-53 | 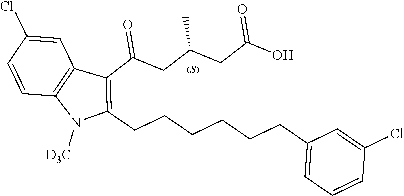 | 455.0 | ¹H NMR (400 MHz, DMSO-d₆) δ 8.41- 8.37 (m, 2H), 7.95 (s, 1H), 7.61-7.56 (m, 2H), 7.29-7.22 (m, 2H), 3.74 (s, 3H), 3.12 (t, J = 7.2 Hz, 2H), 2.97 (dd, J = 16.4, 5.2 Hz, 1H), 2.83 (dd, J = 16.4, 8.0 Hz, 1H), 2.57 (t, J = 7.6 Hz, 2H), 2.48-2.44 (m, 1H), 2.38 (dd, J = 15.2, 4.8 Hz, 1H), 2.16 (dd, J = 15.2, 8.0 Hz, 1H), 1.64-1.49 (m, 4H), 1.47-1.39 (m, 2H), 1.38-1.28 (m, 2H), 0.96 (d, J = 6.0 Hz, 3H). |
| I-54 | 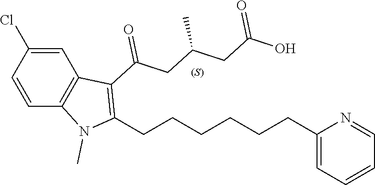 | 455.0 | ¹H NMR (400 MHz, DMSO-d₆) δ 12.25 (s, 1H), 8.46 (d, J = 4.4 Hz, 1H), 7.96 (s, 1H), 7.66 (t, J = 8.0 Hz, 1H), 7.58 (d, J = 8.8 Hz, 1H), 7.25-7.22 (m, 2H), 7.19-7.16 (m, 1H), 3.75 (s, 3H), 3.13 (t, J = 7.6 Hz, 2H), 2.98 (dd, J = 16.4, 5.6 Hz, 1H), 2.83 (dd, J = 16.4, 7.6 Hz, 1H), 2.71 (t, J = 7.6 Hz, 2H), 2.50-2.43 (m, 1H), 2.37 (dd, J = 15.6, 5.6 Hz, 1H), 2.16 (dd, J = 15.2, 8.0 Hz, 1H), 1.76-1.64 (m, 2H), 1.60-1.50 (m, 2H), 1.48- 1.41 (m, 2H), 1.39-1.29 (m, J = 6.8 Hz, 2H), 0.96 (d, J = 6.4 Hz, 3H). |
| I-55 | 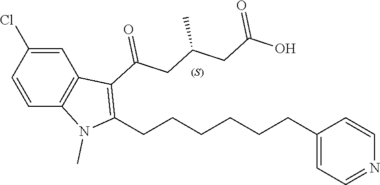 | 455.0 | ¹H NMR (400 MHz, DMSO-d₆) δ 8.42 (d, J = 5.6 Hz, 2H), 7.95 (d, J = 2.0 Hz, 1H), 7.57 (d, J = 8.4 Hz, 1H), 7.23 (dd, J = 8.8, 2.0 Hz, 1H), 7.20 (d, J = 5.6 Hz, 2H), 3.74 (s, 3H), 3.12 (t, J = 8.0 Hz, 2H), 2.98 (dd, J = 16.4, 6.0 Hz, 1H), 2.83 (dd, J = 16.0, 7.6 Hz, 1H), 2.57 (t, J = 7.6 Hz, 2H), 2.49-2.44 (m, 1H), 2.38 (dd, J = 15.4, 5.6 Hz, 1H), 2.16 (dd, J = 15.4, 8.0 Hz, 1H), 1.61-1.51 (m, 4H), 1.45-1.39 (m, 2H), 1.37-1.33 (m, 2H), 0.96 (d, J = 6.8 Hz, 3H). |
| I-57 | 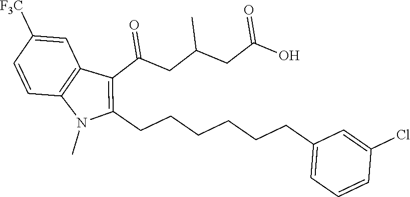 [SMILES: CC(CC(=O)O)CC(=O)c1c(CCCCCCc2cccc(Cl)c2)n(C)c2ccc(C(F)(F)F)cc12] | 522.0 | ¹H NMR (400 MHz, CDCl₃) δ 8.21 (s, 1H), 7.50 (dd, J = 8.8, 1.2 Hz, 1H), 7.41 (d, J = 8.8 Hz, 1H), 7.21-7.12 (m, 3H), 7.03 (d, J = 7.6 Hz, 1H), 3.75 (s, 3H), 3.19 (t, J = 8.0 Hz, 2H), 3.12-2.95 (m, 2H), 2.78-2.69 (m, 1H), 2.61-2.53 (m, 3H), 2.37 (dd, J = 15.6, 7.6 Hz, 1H), 1.68-1.58 (m, 4H), 1.53-1.34 (m, 4H), 1.55 (d, J = 6.8 Hz, 3H). |
| I-70 | 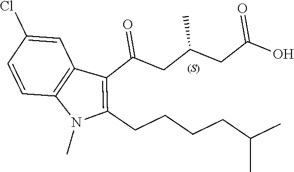 | 392.0 | ¹H NMR (400 MHz, CDCl₃) δ 7.89 (d, J = 1.6 Hz, 1H), 7.21 (d, J = 8.8 Hz, 1H), 7.17 (dd, J = 8.8, 1.6 Hz, 1H), 3.69 (s, 3H), 3.14 (t, J = 8.0 Hz, 2H), 3.01 (dd, J = 16.0, 7.2 Hz, 1H), 2.91 (dd, J = 16.0, 6.8 Hz, 1H), 2.79-2.68 (m, 1H), 2.56 (dd, J = 15.2, 5.6 Hz, 1H), 2.34 (dd, J = 15.2, 7.6 Hz, 1H), 1.62-1.39 (m, 5H), 1.24-1.18 (m, 2H), 1.12 (d, J = 6.8 Hz, 3H), 0.86 (d, J = 6.4 Hz, 6H). |
| I-81 | 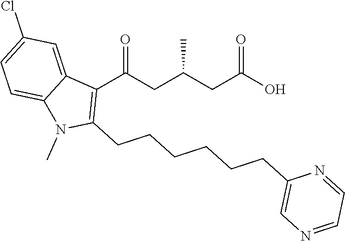 [SMILES: C[C@H](CC(=O)O)CC(=O)c1c(CCCCCCc2cnccn2)n(C)c2ccc(Cl)cc12] | 456.0 | ¹H NMR (400 MHz, CDCl₃) δ 8.55-8.37 (m. 3H), 7.95 (d, J = 1.6 Hz, 1H), 7.24-7.18 (m, 2H), 3.69 (s, 3H), 3.22-3.11 (m, 2H), 3.06 (dd, J = 16.0, 6.8 Hz, 1H), 2.93 (dd, J = 16.0, 6.8 Hz, 1H), 2.83 (t, J = 7.6 Hz, 2H), 2.80-2.70 (m, 1H), 2.53 (dd, J = 15.2, 6.4 Hz, 1H), 2.40 (dd, J = 15.2, 6.4 Hz, 1H), 1.82-1.76 (m, 2H), 1.67-1.56 (m, 2H), 1.54- 1.37 (m, 4H), 1.15 (d, J = 6.8 Hz, 3H). |
| I-84 | 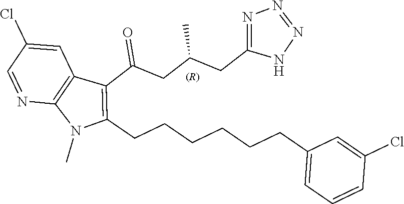 | 513.0 | ¹H NMR (400 MHz, DMSO-d₆) δ 16.05 (s, 1H), 8.35-8.33 (m, 2H), 7.30-7.20 (m, 3H), 7.15 (d, J = 7.6 Hz, 1H), 3.80 (s, 3H), 3.16 (t, J = 7.6 Hz, 2H), 3.11-2.87 (m, 4H), 2.67- 2.62 (m, 1H), 2.57 (t, J = 7.6 Hz, 1H), 1.61- 1.54 (m, 4H), 1.47-1.40 (m, 2H), 1.37-1.33 (m, 2H), 0.93 (d, J = 6.4 Hz, 3H). |
| I-96 | 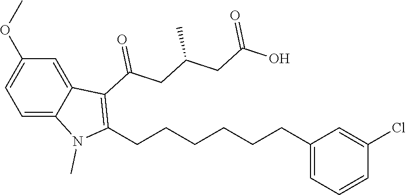 [SMILES: COc1ccc2c(c1)c(C(=O)C[C@H](C)CC(=O)O)c(CCCCCCc1cccc(Cl)c1)n2C] | 484.2 | ¹H NMR (400 MHz, DMSO-d₆) δ 12.06 (s, 1H), 7.45-7.41 (m, 2H), 7.29-7.21 (m, 3H), 7.16 (d, J = 6.4 Hz, 1H), 6.87 (d, J = 8.0 Hz, 1H), 3.81 (s, 3H), 3.71 (s, 3H), 3.13-3.11 (m, 2H), 2.99-2.94 (m, 1H), 2.85-2.79 (m, 1H), 2.59-2.56 (m, 3H), 2.44-2.38 (m, 1H), 2.19-2.14 (m, 1H), 1.58-1.56 (m, 4H), 1.44- 1.41 (m, 2H), 1.35-1.34 (m, 2H), 0.97 (d, J = 5.6 Hz, 3H). |
| I-97 | 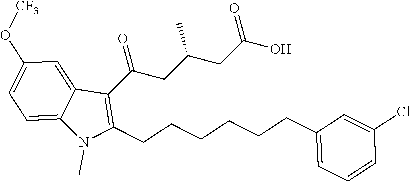 [SMILES: C[C@H](CC(=O)O)CC(=O)c1c(CCCCCCc2cccc(Cl)c2)n(C)c2ccc(OC(F)(F)F)cc12] | 537.9 | ¹H NMR (400 MHz, DMSO-d₆) δ 7.93 (s, 1H), 7.66 (d, J = 8.8 Hz, 1H), 7.28-7.14 (m, 5H), 3.78 (s, 3H), 3.15 (d, J = 7.6 Hz, 2H), 3.00 (dd, J = 16.4, 6.4 Hz, 1H), 2.84 (dd, J = 16.4, 7.6 Hz, 1H), 2.62-2.53 (m, 3H), 2.41 (dd, J = 15.6, 6.0 Hz, 1H), 2.19 (dd, J = 15.6, 8.0 Hz, 1H), 1.62-1.52 (m, 4H), 1.50-1.40 (m, 2H), 1.39-1.30 (m, 2H), 0.99 (d, J = 6.8 Hz, 3H). |
| I-98 | 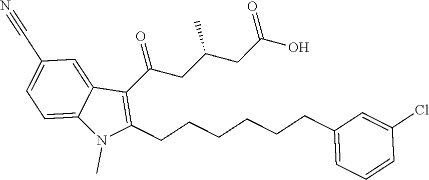 [SMILES: C[C@H](CC(=O)O)CC(=O)c1c(CCCCCCc2cccc(Cl)c2)n(C)c2ccc(C#N)cc12] | 479.0 | ¹H NMR (400 MHz, DMSO-d₆) δ 12.05 (s, 1H), 8.43 (s, 1H), 7.76 (d, J = 8.8 Hz, 1H), 7.62 (dd, J = 8.8, 1.6 Hz, 1H), 7.31-7.21 (m, 3H), 7.16 (d, J = 7.2 Hz, 1H), 3.81 (s, 3H), 3.16 (t, J = 7.6 Hz, 2H), 3.07 (dd, J = 16.4, 5.6 Hz, 1H), 2.91 (dd, J = 16.4, 7.6 Hz, 1H), 2.58 (t, J = 7.6 Hz, 2H), 2.53 (s, 1H), 2.41 (dd, J = 15.6, 6.0 Hz, 1H), 2.18 (dd, J = 15.6, 8.0 Hz, 1H), 1.62-1.56 (m, 4H), 1.48-1.41 (m, 2H), 1.38-1.32 (m, 2H), 0.97 (d, J = 6.4 Hz, 3H). |
| I-144 | 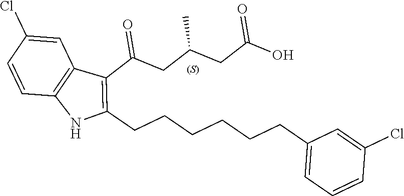 | 474.1 | ¹H NMR (400 MHz, DMSO-d₆) δ 12.06 (s, 1H), 7.96 (d, J = 1.6 Hz, 1H), 7.40 (d, J = 8.8 Hz, 1H), 7.30-7.21 (m, 3H), 7.17-7.13 (m, 2H), 3.35 (br, 1H), 3.06 (t, J = 8.0 Hz, 2H), 2.95 (dd, J = 16.4, 5.6 Hz, 1H), 2.81 (dd, J = 16.4, 7.6 Hz, 1H), 2.56 (t, J = 8.0 Hz, 2H), 2.38 (dd, J = 15.6, 6.0 Hz, 1H), 2.17 (dd, J = 15.6, 8.0 Hz, 1H), 1.71-1.64 (m, 2H), 1.60-1.53 (m, 2H), 1.41-1.32 (m, 4H), 0.97 (d, J = 6.8 Hz, 3H). |
| I-150 | 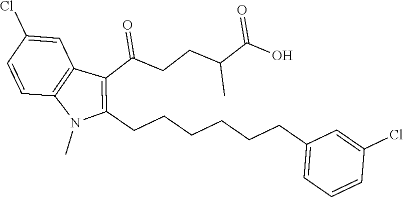 [SMILES: CC(CCC(=O)c1c(CCCCCCc2cccc(Cl)c2)n(C)c2ccc(Cl)cc12)C(=O)O] | 488.2 | ¹H NMR (400 MHz, DMSO-d₆) δ 12.11 (s, 1H), 7.92 (dd, J = 16.8, 2.0 Hz, 1H), 7.59 (dd, J = 8.8, 4.0 Hz, 1H), 7.31- 7.21 (m, 4H), 7.16 (d, J = 7.6 Hz, 1H), 3.75 (d, J = 2.0 Hz, 3H), 3.39-3.37 (m, 1H), 3.14-3.08 (m, 2H), 2.97-2.92 (m, 1H), 2.58 (t, J = 7.6 Hz, 2H), 2.47-2.44 (m, 1H), 2.25- 2.19 (m, 1H), 2.00-1.89 (m, 1H), 1.65-1.52 (m, 4H), 1.47-1.40 (m, 2H), 1.36-1.31 (m, 2H), 1.12 (t, J = 6.0, 3H). |
| I-305 | 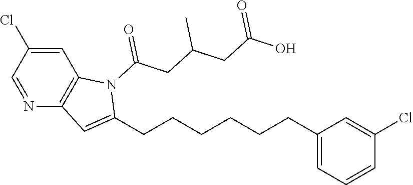 [SMILES: CC(CC(=O)O)CC(=O)n1c(CCCCCCc2cccc(Cl)c2)cc2ncc(Cl)cc21] | 475.1 | ¹H NMR (400 MHz, DMSO-d₆) δ 11.35 (br., 1H), 8.20 (d, J = 2.0 Hz, 1H), 7.71 (d, J = 2.0 Hz, 1H), 7.31-7.21 (m, 3H), 7.15 (d, J = 7.2 Hz, 1H), 6.31 (d, J = 0.8 Hz, 1H), 2.81-2.72 (m, 4H), 2.57 (t, J = 7.6 Hz, 2H), 2.52-2.51 (m, 1H), 2.50-2.46 (m, 1H), 2.33- 2.23 (m, 1H), 1.72-1.65 (m, 2H), 1.60-1.53 (m, 2H), 1.37-1.30 (m, 4H), 0.96 (d, J = 6.8 Hz, 3H). |
| I-468 | 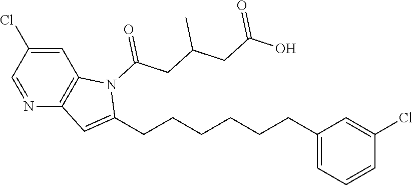 [SMILES: CC(CC(=O)O)CC(=O)n1c(CCCCCCc2cccc(Cl)c2)cc2ncc(Cl)cc21] | — | (DMSO-d₆) δ 11.35 (br., 1H), 8.20 (d, J = 2.0 Hz, 1H), 7.71 (d, J = 2.0 Hz, 1H), 7.31- 7.21 (m, 3H), 7.15 (d, J = 7.2 Hz, 1H), 6.31 (d, J = 0.8 Hz, 1H), 2.81-2.72 (m, 4H), 2.57 (t, J = 7.6 Hz, 2H), 2.52-2.51 (m, 1H), 2.50- 2.46 (m, 1H), 2.33-2.23 (m, 1H), 1.72-1.65 (m, 2H), 1.60-1.53 (m, 2H), 1.37-1.30 (m, 4H), 0.96 (d, J = 6.8 Hz, 3H). |

TABLE 2a
Additional Characterization Data for Additional Exemplary Compounds

| Compound | Rt (Min) | Rt (Min) |
|---|---|---|
| No. | (LCMS) | (HPLC) |
| I-305 | 2.109 | 10.774 |

### Example 2: Synthesis of Compound I-60

### Synthetic Scheme for Compound I-60

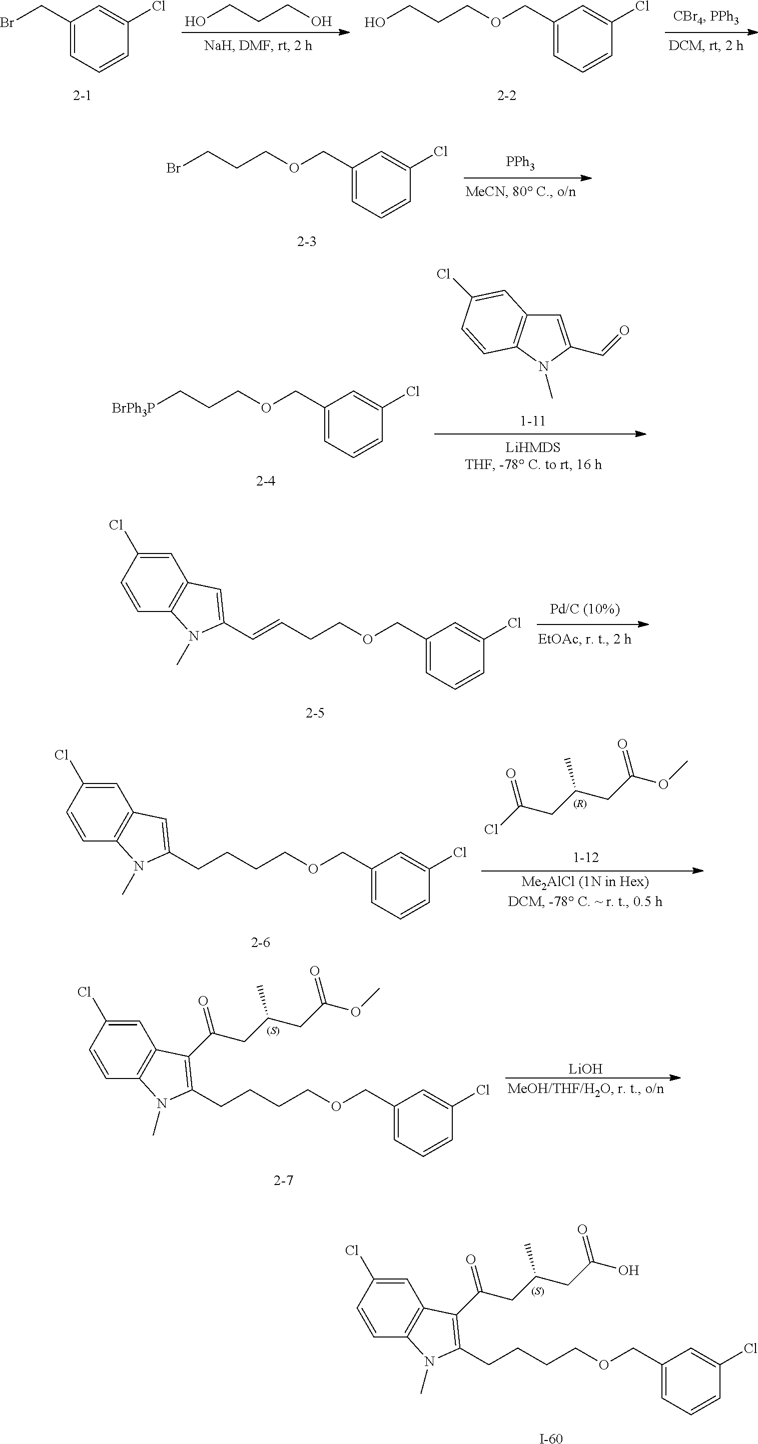

### 2.1 The Synthesis of Intermediate 2-2

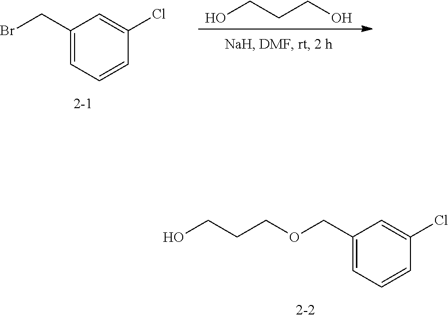

To a stirred solution of propane-1,3-diol (25.9 g, 340.7 mmol) in DMF (150 mL) was added NaH (1.4 g, 34.1 mmol, 60% in mineral oil). The reaction mixture was stirred at 0° C. for 0.5 h under N₂ atmosphere, then 2-1 (7.0 g, 34.1 mmol) was added slowly and the reaction mixture was stirred at room temperature for 2 hours until the reaction was complete. The suspension was diluted with NaCl (aq., 500 mL) and extracted with EtOAc (500 mL×2). The organic layer was washed with brine (500 mL×2), dried over anhydrous Na₂SO₄, filtered and concentrated in vacuo to afford 2-2 (6.8 g, crude) as colorless oil. ¹H NMR (400 MHz, CDCl₃) δ 7.32 (s, 1H), 7.27-7.25 (m, 2H), 7.21-7.19 (m, 1H), 4.48 (d, J=6.8 Hz, 2H), 3.78 (t, J=6.0 Hz, 2H), 3.65 (t, J=6.0 Hz, 2H), 2.38 (s, 1H), 1.88-1.85 (m, 2H).

### 2.2 The Synthesis of Intermediate 2-3

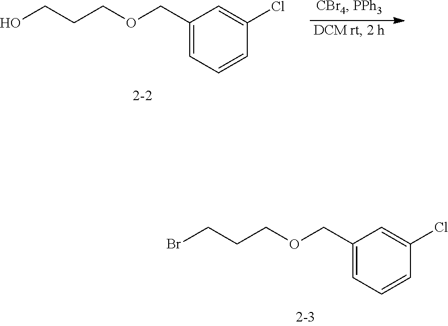

To a stirred solution of 2-2 (6.5 g, 32.5 mmol) in DCM (100 mL) was added CBr₄ (16.2 g, 48.7 mmol) and PPh₃ (8.5 g, 48.7 mmol) at 0° C. The reaction mixture was allowed to warm to room temperature and stirred for 2 hours until the reaction was complete. The solvent was evaporated under reduced pressure and the crude was purified by silica gel chromatography (PE/EA=3/1) to afford 2-3 (5.6 g, yield: 66%) as colorless oil. ¹H NMR (400 MHz, CDCl₃) δ 7.33 (s, 1H), 7.27-7.26 (m, 2H), 7.21 (d, J=0.8 Hz, 1H), 4.49 (s, 2H), 3.61 (t, J=6.0 Hz, 2H), 3.54 (t, J=6.4 Hz, 2H), 2.14 (t, J=6.4 Hz, 2H).

### 2.3 The Synthesis of Intermediate 2-4

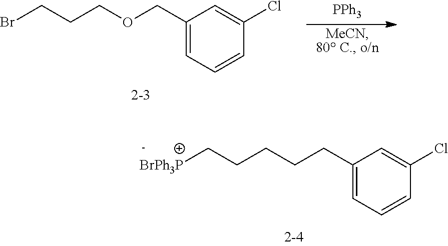

To a stirred solution of 2-3 (5.6 g, 21.3 mmol) in acetonitrile (100 mL) was added PPh₃ (11.1 g, 42.5 mmol). The reaction mixture was reflux at 80° C. overnight. The solvent was evaporated under reduced pressure and the crude was purified by silica gel chromatography (15% MeOH/DCM) to afford 2-4 (11.1 g, yield: 99%) as colorless oil. LC-MS m/z: 445.0 [M−Br]*.

### 2.4 The Synthesis of Intermediate 2-5

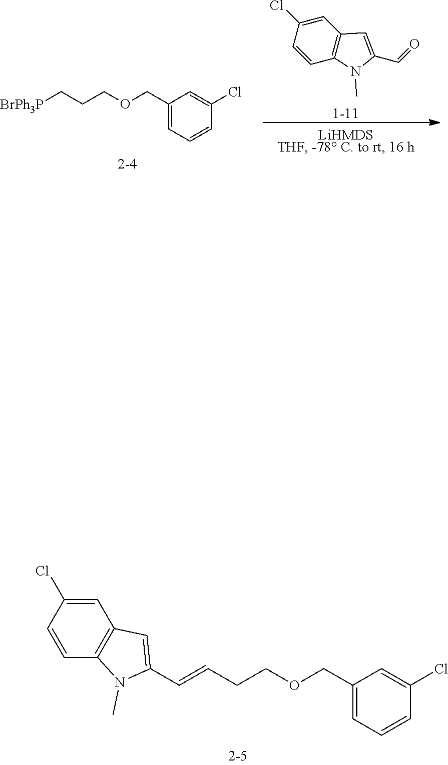

To a suspension of 2-4 (11.0 g, 20.9 mmol) in THF (100 mL) was added LiHMDS (1.6 M in THF, 16 mL, 25.1 mmol) at −78° C. The mixture was stirred for 30 min and 1-11 (4.9 g, 25.1 mmol) in THF (40 ml) was added dropwise. The reaction mixture was allowed to warm to rt and stirred for another 16 h. Saturated NH₄Cl solution was added and the reaction mixture was extracted with EtOAc. The combined organic layer was washed with brine, dried over anhydrous Na₂SO₄ and the solvent was evaporated under reduced pressure. The crude was purified by silica gel chromatography (10% EtOAc/Hexane) to afford 2-5 (5.1 g, yield: 67%) as a yellow solid. LC-MS m/z: 360.0 [M+H]⁺.

### 2.5 The Synthesis of Intermediate 2-6

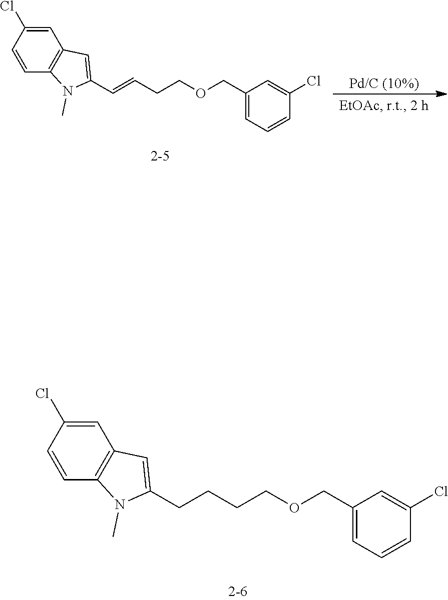 [SMILES: Cn1c(/C=C/CCOCc2cccc(Cl)c2)cc2cc(Cl)ccc21 ; "EtOAc, r.t., 2 h" ; "Pd/C (10%)" ; Cn1c(CCCCOCc2cccc(Cl)c2)cc2cc(Cl)ccc21]

To a stirred solution of 2-5 (2.1 g, 5.6 mmol) in EtOAc (20 mL) was added 10% Pd/C (500 mg) under H₂ atmosphere (1.0 atm). The reaction mixture was stirred at room temperature for 2 h until the reaction was complete (by LCMS) then filtered. The residue was washed with EtOAc and the combined filtrate was concentrated under reduced pressure to afford the crude product 2-6 (1.4 g, yield: 66%) as colorless oil. LC-MS m/z: 362.0 [M+H]⁺.

### 2.6 The Synthesis of Intermediate 2-7

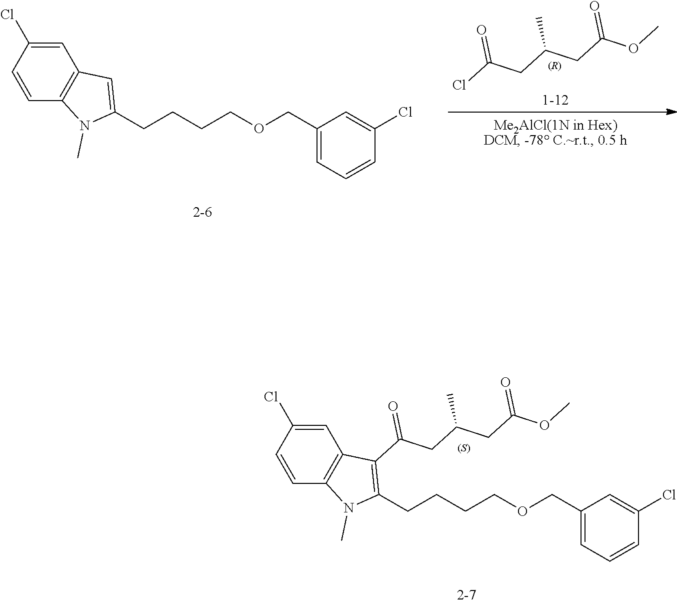

To a stirred solution of 2-6 (300 mg, 0.83 mmol) in dichloromethane (5 mL) was added Me₂AlCl (1.0 M in hexane, 1.7 mL, 1.66 mmol) at −78° C. After 30 min, 1-12 (222 mg, 1.24 mmol) in dichloromethane (1 mL) was added dropwise and the reaction mixture was stirred at room temperature for another 0.5 h. The reaction was quenched by adding water and extracted with EtOAc. The organic layer was combined, washed with brine and dried over Na₂SO₄. The solvent was evaporated under reduced pressure and the crude was purified by silica gel chromatography (40% EtOAc/Hex) to afford 2-7 (350 mg, yield: 84%) as colorless oil. LC-MS m/z: 504.0 [M+H]⁺.

### 2.7 The Synthesis of Compound I-60

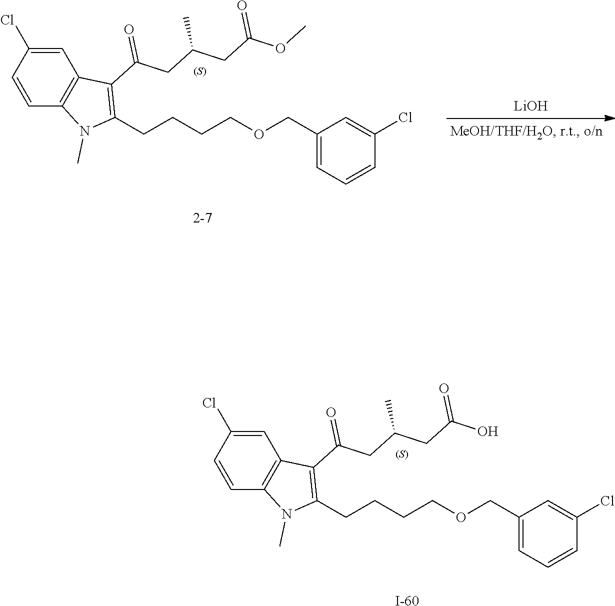

To a stirred solution of 2-7 (350 mg, 0.694 mmol) in MeOH/THF/H₂O (5 mL/5 mL/0.1 mL) was added lithium hydroxide monohydrate (292 mg, 6.9 mmol). The reaction mixture was stirred at rt overnight until the reaction was complete (by LCMS). Saturated NH₄Cl solution was added at 0° C. and the mixture was acidified with HCl (1.0 M) to pH ˜3.0 then extracted with EtOAc, the combined organic layer was washed with brine, dried over anhydrous Na₂SO₄ and evaporated under reduced pressure. The crude was purified by Prep-HPLC to afford I-60 (76 mg, yield: 22%) as colorless semi-solid. ¹H NMR (400 MHz, CDCl₃) δ 7.86 (d, J=1.6 Hz, 1H), 7.30 (s, 1H), 7.24-7.20 (m, 4H), 7.19-7.17 (m, 1H), 4.46 (s, 2H), 3.69 (s, 3H), 3.53 (t, J=6.0 Hz, 2H), 3.23-3.19 (m, 2H), 3.01-2.99 (m, 2H), 2.73-2.71 (m, 1H), 2.51 (dd, J=15.2, 5.6 Hz, 1H), 2.36 (dd, J=14.8, 6.8 Hz, 1H), 1.77-1.73 (m, 4H), 1.15 (d, J=6.8 Hz, 3H). LC-MS m/z: 489.9 [M+H]⁺.

Additional exemplary compounds were prepared following methods substantially similar to the method described above and herein. Data for these compounds are provided in Table 3 below.

TABLE 3
Characterization Data for Additional Exemplary Compounds

| Compound | | | |
|---|---|---|---|
| No. | Chemical Structure | M + 1 | ¹H NMR (400 MHz) |
| I-59 | 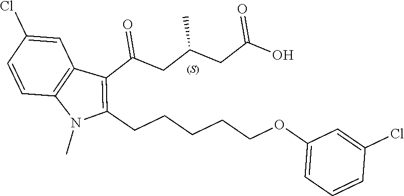 | 489.9 | ¹H NMR (400 MHz, CDCl₃) δ 7.86 (d, J = 1.6 Hz, 1H), 7.23-7.19 (m, 2H), 7.16 (t, J = 8.0 Hz, 1H), 6.90-6.88 (m, 1H), 6.86 (t, J = 2.4 Hz, 1H), 6.75 (dd, J = 8.4, 2.0 Hz, 1H), 3.94 (t, J = 6.4 Hz, 1H), 3.71 (s, 3H), 3.20 (t, J = 7.6 Hz, 2H), 3.03 (dd, J = 16.0, 7.2 Hz, 1H), 2.94 (dd, J = 16.0, 6.4 Hz, 1H), 2.75-2.68 (m, 1H), 2.55 (dd, J = 14.8, 6.0 Hz, 1H), 2.35 (dd, J = 15.2, 7.2 Hz, 1H), 1.87-1.81 (m, 2H), 1.73-1.60 (m, 4H), 1.13 (d, J = 6.4 Hz, 3H). |
| I-61 | 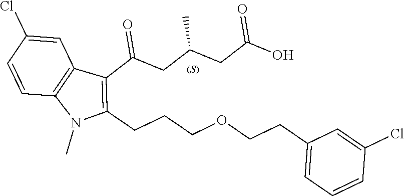 | 489.9 | ¹H NMR (400 MHz, DMSO-d₆) δ 7.97 (d, J = 2.0 Hz, 1H), 7.58 (d, J = 8.4 Hz, 1H), 7.32-7.21 (m, 5H), 3.71 (s, 3H), 3.60 (t, J = 6.4 Hz, 2H), 3.47 (t, J = 6.0 Hz, 2H), 3.14 (t, J = 7.6 Hz, 2H), 2.99 (dd, J = 16.4, 6.0 Hz, 1H), 2.87-2.80 (m, 3H), 2.55-2.53 (m, 1H), 2.37 (dd, J = 15.6, 5.6 Hz, 1H), 2.15 (dd, J = 15.2, 8.0 Hz, 1H), 1.84-1.77 (m, 2H), 0.97 (d, J = 6.4 Hz, 3H). |
| I-62 | 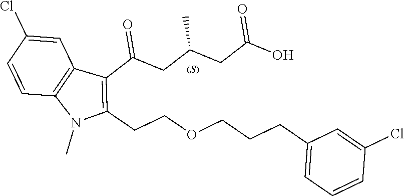 | 489.9 | ¹H NMR (400 MHz, DMSO-d₆) δ 12.10 (s, 1H), 7.96 (d, J = 2.0 Hz, 1H), 7.63 (d, J = 8.8 Hz, 1H), 7.26 (dd, J = 8.8, 2.0 Hz, 1H), 7.21-7.18 (m, 2H), 7.08 (s, 1H), 6.98- 6.95 (m, 1H), 3.81 (s, 3H), 3.65 (t, J = 6.4 Hz, 2H), 3.42 (t, J = 6.4 Hz, 2H), 3.31 (t, J = 6.4 Hz, 2H), 3.03 (dd, J = 16.4, 5.6 Hz, 1H), 2.89 (dd, J = 16.8, 7.6 Hz, 1H), 2.50- 2.46 (m, 3H), 2.39 (dd, J = 15.6, 6.0 Hz, 2.17 (dd, J = 15.2, 8.0 Hz, 1H), 1.74-1.68 (m, 2H), 0.97 (d, J = 6.4 Hz, 3H). |
| I-63 | 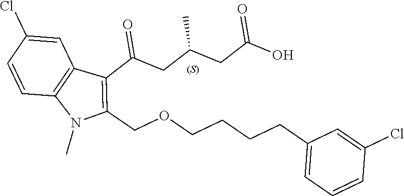 | 489.9 | ¹H NMR (400 MHz, CDCl₃) δ 7.91 (d, J = 1.2 Hz, 1H), 7.28-7.23 (m, 3H), 7.17-7.10 (m, 3H), 6.97 (d, J = 7.2 Hz, 1H), 5.06 (s, 2H), 3.78 (s, 3H), 3.53 (t, J = 6.0 Hz, 2H), 3.06 (dd, J = 16.0, 6.8 Hz, 1H), 2.93 (dd, J = 16.0, 6.8 Hz, 1H), 2.75-2.70 (m, 1H), 2.56-2.51 (m, 3H), 2.33 (dd, J = 15.4, 7.2 Hz, 1H), 1.65-1.57 (m, 4H), 1.10 (d, J = 6.8 Hz, 3H). |
| I-64 | 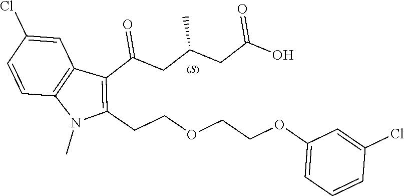 | 492.0 | ¹H NMR (400 MHz, DMSO-d₆) δ 12.05 (s, 1H), 7.93 (d, J = 2.0 Hz, 1H), 7.60 (d, J = 8.8 Hz, 1H), 7.28-7.24 (m, 2H), 6.98- 6.93 (m, 2H), 6.83 (dd, J = 8.0, 1.6 Hz, 1H), 4.05 (t, J = 4.4 Hz, 2H), 3.78-3.68 (m, 5H), 3.42 (t, J = 6.4 Hz, 2H), 3.02 (dd, J = 16.4, 5.6 Hz, 1H), 2.88 (dd, J = 16.4, 7.6 Hz, 1H), 2.49 (s, 1H), 2.39 (dd, J = 15.6, 5.6 Hz, 1H), 2.16 (dd, J =15.2, 8.0 Hz, 1H), 0.97 (d, J = 6.8 Hz, 3H). |
| I-65 | 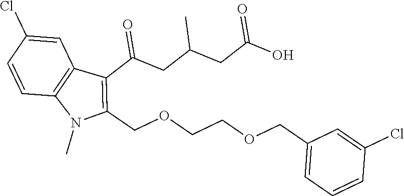 [SMILES: CC(CC(=O)O)CC(=O)c1c(COCCOCc2cccc(Cl)c2)n(C)c2ccc(Cl)cc12] | 491.9 | ¹H NMR (400 MHz, CDCl₃) δ 7.92 (s, 1H), 7.30-7.27 (m, 3H), 7.23 (d, J = 4.8 Hz, 2H), 7.15 (d, J = 4.0 Hz, 1H), 5.20- 5.13 (m, 2H), 4.49 (s, 2H), 3.82 (s, 3H), 3.74-3.72 (m, 2H), 3.65-3.62 (m, 2H), 3.08 (dd, J = 16.0, 6.8 Hz, 1H), 2.96 (dd, J = 16.0, 6.4 Hz, 1H), 2.78-2.69 (m, 1H), 2.53 (dd, J = 15.2, 6.0 Hz, 1H), 2.35 (dd, J = 15.2, 7.2 Hz, 1H), 1.13 (d, J = 6.8 Hz, 3H). |
| I-71 | 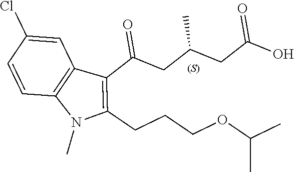 | 394.0 | ¹H NMR (400 MHz, DMSO-d₆) δ 12.03 (s, 1H), 7.95 (s, 1H), 7.55 (d, J = 8.8 Hz, 1H), 7.21 (d, J = 8.4 Hz, 1H), 3.73 (s, 3H), 3.55-3.46 (m, 1H), 3.39 (t, J = 6.0 Hz, 2H), 3.14 (t, J = 7.2 Hz, 2H), 2.94 (dd, J = 16.4, 5.6 Hz, 1H), 2.82 (dd, J = 16.0, 7.2 Hz, 1H), 2.51-2.43 (m, 1H), 2.35 (dd, J = 15.6, 5.6 Hz, 1H), 2.12 (dd, J = 15.2, 8.0 Hz, 1H), 1.78-1.72 (m, 2H), 1.05 (d, J = 6.0 Hz, 6H), 0.94 (d, J = 6.4 Hz, 3H). |
| I-72 | 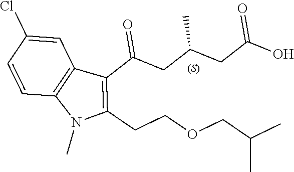 | 394.0 | ¹H NMR (400 MHz, CDCl₃) δ7.79 (d, J = 1.6 Hz, 1H), 7.22 (d, J = 8.8 Hz, 1H), 7.18- 7.17 (m, 1H), 3.73 (s, 3H), 3.69 (t, J = 5.6 Hz, 2H), 3.38 (t, J = 5.2 Hz, 2H), 3.04 (d, J = 6.4 Hz, 2H), 2.98-2.95 (m, 2H) 2.68- 2.63 (m, 1H), 2.49 (dd, J = 15.2, 5.6 Hz, 1H), 2.30 (dd, J = 14.8, 6.8 Hz, 1H), 1.71- 1,64 (m, 1H), 1.10 (d, J = 6.8 Hz, 3H), 0.73 (d, J = 6.4 Hz, 6H). |
| I-73 | 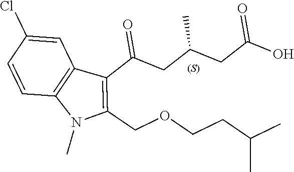 | 394.1 | ¹H NMR (400 MHz, DMSO-d₆) δ 12.06 (s, 1H), 8.03 (d, J = 2.0 Hz, 1H), 7.61 (d, J = 8.8 Hz, 1H), 7.29 (dd, J = 8.8, 2.0 Hz, 1H), 4.95 (s, 2H), 3.79 (s, 3H), 3.49 (t, J = 6.4 Hz, 2H), 3.01 (dd, J = 16.4, 5.6 Hz, 1H), 2.87 (dd, J = 16.4, 7.6 Hz, 1H), 2.47- 2.45 (m, 1H), 2.34 (dd, J = 15.6, 6.0 Hz, 1H), 2.12 (dd, J = 15.6, 8.4 Hz, 1H), 1.64- 1.57 (m, 1H), 1.38 (q, J = 6.8 Hz, 2H), 0.93 (d, J = 6.4 Hz, 3H), 0.80 (d, J = 6.4 Hz, 6H). |
| I-74 | 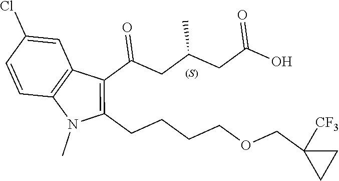 | 487.9 | ¹H NMR (400 MHz, CDCl₃) δ 7.87 (d, J = 1.6 Hz, 1H), 7.26-7.20 (m, 2H), 3.71 (s, 3H), 3.53-3.48 (m, 4H), 3.20 (t, J = 7.2 Hz, 2H), 3.05-2.93 (m, 2H), 2.74-2.70 (m, 1H), 2.54 (dd, J = 14.8, 5.6 Hz, 2H), 2.36 (dd, J = 15.2, 7.2 Hz, 2H), 1.72-1.68 (m, 4H), 1.14 (d, J = 6.4 Hz, 3H), 1.00-0.97 (m, 2H), 0.75-0.72 (m, 2H). |
| I-83 | 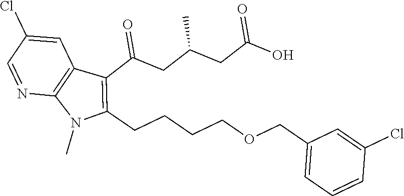 [SMILES: C[C@H](CC(=O)O)CC(=O)c1c(CCCCOCc2cccc(Cl)c2)n(C)c2ncc(Cl)cc12] | 490.9 | ¹H NMR (400 MHz, DMSO-d₆ ) δ 12.10 ((s, 1H), 8.38 (d, J = 2.4 Hz, 1H), 8.33 (d, J = 2.0 Hz, 1H), 7.40-7.31 (m, 3H), 7.28 (d, J = 7.2 Hz, 1H), 4.47 (s, 2H), 3.80 (s, 3H), 3.50 (t, J = 6.0 Hz, 2H), 3.22 (t, J = 7.2 Hz, 2H), 3.04 (dd, J = 16.6, 5.6 Hz, 1H), 2.87 (dd, J = 16.6, 7.6 Hz, 1H), 2.49- 2.44 (m, 1H), 2.40 (dd, J = 15.2, 5.6 Hz, 1H), 2.18 (dd, J = 15.2, 7.6 Hz, 1H), 1.76- 1.59 (m, 4H), 0.96 (d, J = 6.8 Hz, 3H). |
| I-99 | 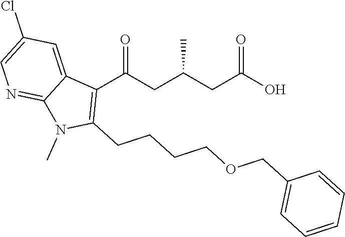 [SMILES: C[C@H](CC(=O)O)CC(=O)c1c(CCCCOCc2ccccc2)n(C)c2ncc(Cl)cc12] | 457.0 | ¹H NMR (400 MHz, CDCl₃) δ 8.25 (d, J = 2.0 Hz, 1H), 8.18 (d, J = 2.0 Hz, 1H), 7.33- 7.29 (m, 4H), 7.28-7.23 (m, 1H), 4.51 (s, 2H), 3.79 (s, 3H), 3.55 (d, J = 5.6 Hz, 2H), 3.27-3.20 (m, 2H), 3.04 (d, J = 16.0, 6.4 Hz, 1H), 2.86 (d, J = 16.0, 7.2 Hz, 1H), 2.78-2.69 (m, 1H), 2.53 (dd, J = 15.2, 6.4 Hz, 1H), 2.37 (dd, J = 15.2, 7.2 Hz, 1H), 1.82-1.71 (m, 4H), 1.13 (d, J = 6.8 Hz, 1H). |
| I-100 | 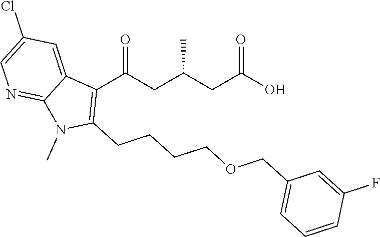 [SMILES: C[C@H](CC(=O)O)CC(=O)c1c(CCCCOCc2cccc(F)c2)n(C)c2ncc(Cl)cc12] | 474.9 | ¹H NMR (400 MHz, CDCl₃) δ 8.24 (d, J = 2.0 Hz, 1H), 8.18 (d, J = 2.0 Hz, 1H), 7.28- 7.25 (m, 1H), 7.08-7.02 (m, 1H), 7.03 (d, J = 9.6 Hz, 1H), 6.94 (dt, J = 8.4, 2.4 Hz, 1H), 4.49 (s, 2H), 3.81 (s, 3H), 3.55 (t, J = 5.6 Hz, 2H), 3.26-3.22 (m, 2H), 3.05 (dd, J = 16.0, 6.4 Hz, 1H), 2.86 (dd, J = 16.0, 7.2 Hz, 1H), 2.78-2.68 (m, 1H), 2.55 (dd, J = 15.6, 6.0 Hz, 1H), 2.38 (dd, J = 15.6, 6.8 Hz, 1H), 1.85-1.70 (m, 4H), 1.13 (d, J = 6.4 Hz, 3H). |
| I-101 | 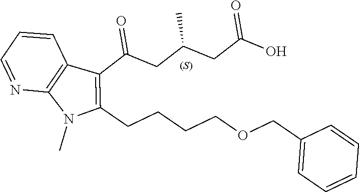 | 423.0 | ¹H NMR (400 MHz, DMSO-d₆) δ 12.03 (s, 1H), 8.31-8.29 (m, 2H), 7.36-7.25 (m, 6H), 4.46 (s, 2H), 3.81 (s, 3H), 3.50 (t, J = 5.6 Hz, 2H), 3.24 (t, J = 6.8 Hz, 2H), 3.02 (dd, J = 16.4, 5.6 Hz, 1H), 2.87 (dd, J = 16.4, 7.6 Hz, 1H), 2.47-2.46 (m, 1H), 2.40 (dd, J = 15.2, 5.6 Hz, 1H), 2.17 (dd, J = 15.2, 8.0 Hz, 1H), 1.69-1.67 (m, 4H), 0.97 (d, J = 6.4 Hz, 3H). |
| I-103 | 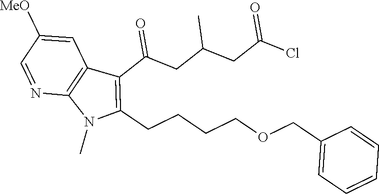 [SMILES: COc1cnc2c(c1)c(C(=O)CC(C)CC(=O)Cl)c(CCCCOCc1ccccc1)n2C] | 453.3 | ¹H NMR (400 MHz, CDCl₃) δ 8.02 (s, 1H), 7.26-7.23 (m, 3H), 7.18-7.15 (m, 3H), 7.11 (s, 1H), 4.05 (t, J = 5.2 Hz, 4H), 3.78 (s, 6H), 2.79 (t, J = 7.6 Hz, 2H), 2.43- 2.35 (m, 3H), 2.25-2.23 (m, 2H), 1.72- 1.56(m, 4H), 1.03 (d, J = 6.0 Hz, 3H). |
| I-108 | 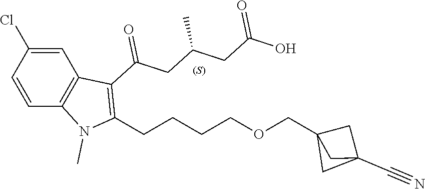 | 471.0 | ¹H NMR (400 MHz, MeOD) δ7.92 (d, J = 2.0 Hz, 1H), 7.43 (d, J = 8.8 Hz, 1H), 7.21 (dd, J = 8.8, 2.0 Hz, 1H), 3.76 (s, 3H), 3.50 (t, J = 5.6 Hz, 2H), 3.41 (s, 2H), 3.22 (t, J = 7.2 Hz, 2H), 3.05 (dd, J = 16.0, 6.4 Hz, 1H), 2.89 (dd, J = 15.6, 7.2 Hz, 1H), 2.69- 2.60 (m, 1H), 2.47 (dd, J = 15.2, 6.0 Hz, 1H), 2.27 (dd, J = 15.2, 7.6 Hz, 1H), 2.18 (s, 6H), 1.71-1.69 (m, 4H), 1.08 (d, J = 6.4 Hz, 3H). |
| I-130 | 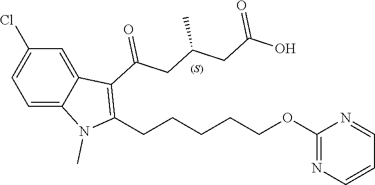 | 458.0 | ¹H NMR (400 MHz, DMSO-d₆) δ 12.08 (s, 1H), 8.58 (d, J = 4.8 Hz, 2H), 7.96 (d, J = 1.6 Hz, 1H), 7.59 (d, J = 8.8 Hz, 1H), 7.25 (dd, J = 8.8, 2.0 Hz, 1H), 7.11 (t, J = 4.8 Hz, 1H), 4.31 (t, J = 6.4 Hz, 2H), 3.77 (s, 3H), 3.23-3.11 (m, 2H), 2.99 (dd, J = 16.4, 5.6 Hz, 1H), 2.85 (dd, J = 16.4, 8.0 Hz, 1H), 2.50-2.44 (m, 1H) 2.38 (dd, J = 15.2, 5.6 Hz, 1H), 2.16 (dd, J = 15.2, 8.0 Hz, 1H), 1.89-1.68 (m, 2H), 1.68-1.41 (m, 4H), 0.96 (d, J = 6.8 Hz, 3H). |
| I-131 | 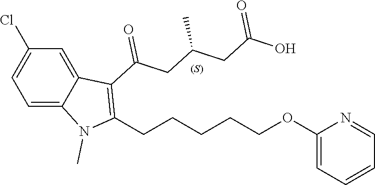 | 456.9 | ¹H NMR (400 MHz, MeOD) δ 8.07 (dd, J = 1.2 Hz, 4.8 Hz, 1H), 7.94 (d, J = 1.6 Hz, 1H), 7.67-7.63 (m, 1H), 7.44 (d, J = 8.4 Hz, 1H), 7.22 (dd, J = 8.8, 1.6 Hz, 1H), 6.93-6.90 (m, 1H), 6.76 (d, J = 8.4 Hz, 1H), 4.27 (t, J = 6.4 Hz, 2H), 3.77 (s, 3H), 3.25 (t, J = 7.2 Hz, 2H), 3.05 (dd, J = 15.6, 6.0 Hz, 1H), 2.89 (dd, J = 16.0, 7.6 Hz, 1H), 2.68-2.60 (m, 1H), 2.36 (dd, J = 15.2, 6.0 Hz, 1H), 2.26 (dd, J = 15.2, 7.6 Hz, 1H), 1.89-1.82 (m, 2H), 1.77-1.62 (m, 4H), 1.06 (d, J = 6.8 Hz, 3H). |
| I-132 | 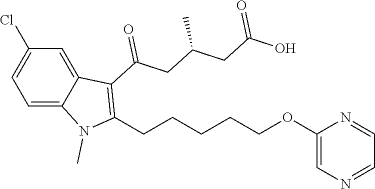 [SMILES: C[C@H](CC(=O)O)CC(=O)c1c(CCCCCOc2cnccn2)n(C)c2ccc(Cl)cc12] | 458.0 | ¹H NMR (400 MHz, DMSO-d₆) δ 12.07 (s, 1H), 8.29 (d, J = 0.8 Hz, 1H), 8.18 (s, 2H), 7.95 (d, J = 1.6 Hz, 1H), 7.59 (d, J = 8.8 Hz, 1H), 7.25 (dd, J = 8.4, 1.6 Hz, 1H), 4.32 (t, J = 6.4 Hz, 2H), 3.77 (s, 3H), 3.18 (t, J = 7.2 Hz, 2H), 2.99 (dd, J = 16.4, 6.0 Hz, 1H), 2.85 (dd, J = 16.4, 7.2 Hz, 1H), 2.50 (s, 1H), 2.39 (dd, J = 15.2, 5.6 Hz, 1H), 2.17 (dd, J = 15.6, 8.0 Hz, 1H), 1.85-1.78 (m, 2H), 1.66-1.56 (m, 4H), 0.96 (d, J = 6.8 Hz, 3H). |
| I-180 | 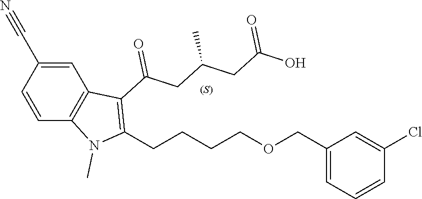 | 481.0 | ¹H NMR (400 MHz, DMSO-d₆) δ 8.43 (d, J = 0.8 Hz, 1H), 7.75 (d, J = 8.8 Hz, 1H), 7.61 (dd, J = 8.4, 1.2 Hz, 1H), 7.39-7.27 (m, 4H), 4.47 (s, 2H), 3.80 (s, 3H), 3.50 (t, J = 5.6 Hz, 2H), 3.20 (t, J = 7.2 Hz, 2H), 3.08 (dd, J = 16.4, 5.6 Hz, 1H), 2.86 (dd, J = 16.4, 8.0 Hz, 1H), 2.47 (d, J = 7.2 Hz, 1H), 2.35 (dd, J = 15.2, 5.6 Hz, 1H), 2.14 (dd, J = 15.2, 8.0 Hz, 1H), 1.72-1.65 (m, 4H), 0.95 (d, J = 6.8 Hz, 3H). |
| I-182 | 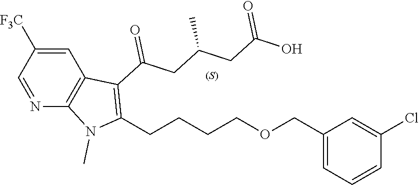 | 525.3 | ¹H NMR (400 MHz, DMSO-d₆) δ 12.11 (s, 1H), 8.68 (d, J = 1.2 Hz, 1H), 8.62 (d, J = 1.6 Hz, 1H), 7.39-7.32 (m, 3H), 7.29- 7.27 (m, 1H), 4.48 (s, 2H), 3.87 (s, 3H), 3.52 (t, J = 5.6 Hz, 2H), 3.26 (t, J = 7.6 Hz, 2H), 3.10 (dd, J = 16.8, 5.6 Hz, 1H), 2.91 (dd, J = 16.8, 8.0 Hz, 1H), 2.51-2.46 (m, 1H), 2.40 (dd, J = 15.6, 6.0 Hz, 1H), 2.20 (dd, J = 15.6, 8.0 Hz, 1H), 1.72-1.71 (m, 4H), 0.96 (d, J = 6.8 Hz, 3H). |
| I-185 | 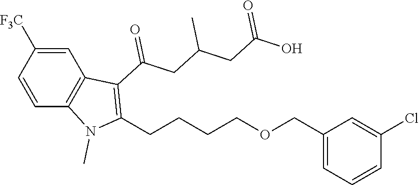 [SMILES: CC(CC(=O)O)CC(=O)c1c(CCCCOCc2cccc(Cl)c2)n(C)c2ccc(C(F)(F)F)cc12] | 524.2 | ¹H NMR (400 MHz, DMSO-d₆) δ 12.08 (s, 1H), 8.32 (s, 1H), 7.77 (d, J = 8.8 Hz, 1H), 7.54 (d, J = 8.0 Hz, 1H), 7.39-7.27 (m, 4H), 4.48 (s, 2H), 3.82 (s, 3H), 3.51 (t, J = 5.6 Hz, 2H), 3.21 (t, J = 8.0 Hz, 2H), 3.04 (dd, J = 16.4, 5.6 Hz, 1H), 2.90 (dd, J = 16.4, 7.6 Hz, 1H), 2.49 (s, 1H), 2.39 (dd, J = 15.2, 5.6 Hz, 1H), 2.18 (dd, J = 15.2, 8.0 Hz, 1H), 1.75-1.67 (m, 4H), 0.97 (d, J = 6.4 Hz, 3H). |
| I-189 | 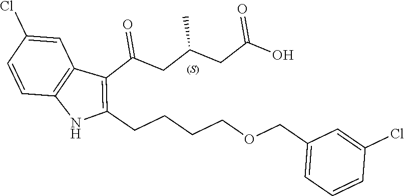 | 475.9 | ¹H NMR (400 MHz, DMSO-d₆) δ 12.06 (s, 1H), 7.95 (d, J = 2.0 Hz, 1H), 7.41-7.31 (m, 4H), 7.27 (d, J = 7.2 Hz, 1H), 7.16 (dd, J = 8.4, 2.0 Hz, 1H), 4.45 (s, 2H), 3.47 (t, J = 6.0 Hz, 2H), 3.09 (t, J = 7.6 Hz, 2H), 2.96 (dd, J = 16.4, 5.6 Hz, 1H), 2.79 (dd, J = 16.0, 7.6 Hz, 1H), 2.46-2.43 (m, 1H), 2.34 (dd, J = 15.2, 5.6 Hz, 1H), 2.13 (dd, J = 15.2, 8.0 Hz, 1H), 1.80-1.72 (m, 2H), 1.66-1.59 (m, 2H), 0.96 (d, J = 6.8 Hz, 3H). |
| I-279 | 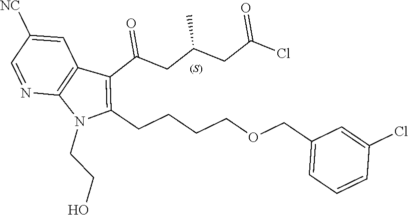 | 510.3 | ¹H NMR (400 MHz, DMSO-d₆) δ 12.12 (br., 1H), 8.83 (s, 1H), 8.72 (d, J = 1.2 Hz, 1H), 7.31-7.26 (m, 2H), 7.22 (d, J = 8.0 Hz, 1H), 7.16 (d, J = 7.2 Hz, 1H), 5.00 (br., 1H), 4.39 (t, J = 5.6 Hz, 2H), 3.74 (t, J = 5.6 Hz, 2H), 3.24 (t, J = 8.0 Hz, 2H), 3.11 (dd, J = 17.2, 6.8 Hz, 1H), 2.92 (dd, J = 16.8, 8.0 Hz, 1H), 2.59 (t, J = 8.0 Hz, 2H), 2.41 (dd, J = 16.0, 8.0 Hz, 1H), 2.18 (dd, J = 15.6, 8.0 Hz, 1H), 1.60-1.57 (m, 4H), 1.48-1.44 (m, 2H), 1.40-1.33 (m, 2H), 1.23 (br., 1H), 0.97 (t, J = 6.4 Hz, 3H). |
| I-280 | 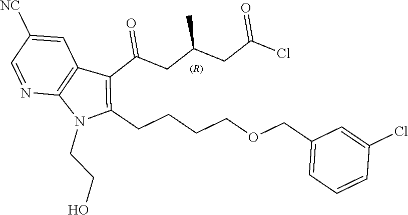 | 510.3 | ¹H NMR (400 MHz, DMSO-d₆) δ 12.12 (br., 1H), 8.83 (s, 1H), 8.71 (s, 1H), 7.31- 7.26 (m, 2H), 7.21 (d, J = 8.0 Hz, 1H), 7.16 (d, J = 7.6 Hz, 1H), 5.00 (br., 1H), 4.38 (t, J = 5.6 Hz, 2H), 3.74 (t, J = 5.2 Hz, 2H), 3.24 (t, J = 8.0 Hz, 2H), 3.11 (dd, J = 17.2, 6.8 Hz, 1H), 2.92 (dd, J = 16.8, 8.0 Hz, 1H), 2.59 (t, J = 8.0 Hz, 2H), 2.41 (dd, J = 16.0, 8.0 Hz, 1H), 2.18 (dd, J = 15.6, 8.0 Hz, 1H), 1.60-1.57 (m, 4H), 1.48-1.44 (m, 2H), 1.40-1.33 (m, 2H), 1.23 (br., 1H), 0.97 (t, J = 6.4 Hz, 3H). |
| I-281 | 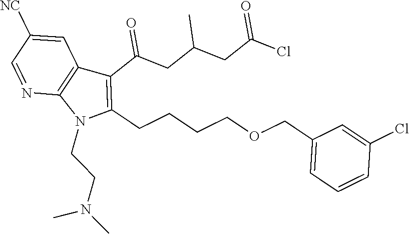 [SMILES: CC(CC(=O)Cl)CC(=O)c1c(CCCCOCc2cccc(Cl)c2)n(CCN(C)C)c2ncc(C#N)cc12] | 537.3 | ¹H NMR (400 MHz, DMSO-d₆) δ 12.09 (s, 1H), 8.82 (d, J = 1.6 Hz, 1H), 8.73 (d, J = 2.0 Hz, 1H), 7.31-7.26 (m, 2H), 7.23- 7.21 (m, 1H), 7.17-7.15 (m, 1H), 4.41 (t, J = 6.4 Hz, 2H), 3.17 (t, J = 7.6 Hz, 2H), 3.11 (dd, J = 16.8, 5.6 Hz, 1H), 2.93 (dd, J = 17.2, 7.6 Hz, 1H), 2.61-2.57 (m, 4H), 2.51-2.47 (m, 1H), 2.42 (dd, J = 15.6, 6.0 Hz, 1H), 2.21-2.15 (m, 7H), 1.61-1.56 (m, 4H), 1.49-1.45 (m, 2H), 1.39-1.34 (m, 2H), 0.97 (d, J = 6.8 Hz, 3H). |
| I-282 | 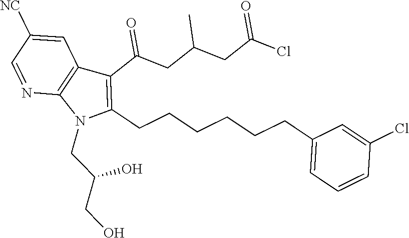 [SMILES: CC(CC(=O)Cl)CC(=O)c1c(CCCCCCc2cccc(Cl)c2)n(C[C@H](O)CO)c2ncc(C#N)cc12] | 540.2 | ¹H NMR (400 MHz, DMSO-d₆) δ 12.14 (br., 1H), 8.82 (d, J = 1.6 Hz, 1H), 8.71 (d, J = 2.0 Hz, 1H), 7.31-7.15 (m, 4H), 5.05 (br., 1H), 4.89-4.81 (m, 1H), 4.52 (dd, J = 14.0, 3.2 Hz, 1H), 4.18-4.12 (m, 1H), 3.92-3.89 (m, 1H), 347-3.39 (m, 3H), 3.26-3.22 (m, 1H), 3.15-3.07 (m, 1H), 2.96-2.88 (m, 1H), 2.58 (t, J = 7.6 Hz, 2H), 2.54-2.52 (m, 1H), 2.44-2.38 (m, 1H), 2.21-2.14 (m, 1H), 1.60-1.53 (m, 4H), 1.48-1.41 (m, 2H), 1.37-1.32 (m, 2H), 0.97 (dd, J = 6.8, 3.2 Hz, 3H). |
| I-284 | 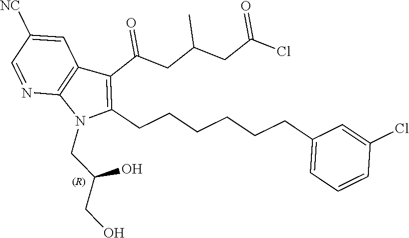 | 540.2 | ¹H NMR (400 MHz, DMSO-d₆) δ 12.09 (br., 1H), 8.82 (d, J = 1.2 Hz, 1H), 8.72 (d, J = 2.0 Hz, 1H), 7.31-7.15 (m, 4H), 5.01 (d, J = 4.8 Hz, 1H), 4.85 (t, J = 5.2 Hz, 1H), 4.52 (dd, J = 14.0, 2.0 Hz, 1H), 4.18- 4.12 (m, 1H), 3.94-3.87 (m, 1H), 348-3.40 (m, 3H), 3.28-3.19 (m, 1H), 3.14-3.07 (m, 1H), 2.97-2.89 (m, 1H), 2.58 (t, J = 7.6 Hz, 2H), 2.54-2.52 (m, 1H), 2.46-2.39 (m, 1H), 2.22-2.16 (m, 1H), 1.62-1.55 (m, 4H), 1.48-1.41 (m, 2H), 1.38-1.31 (m, 2H), 0.97 (dd, J = 6.4, 2.8 Hz, 3H). |
| I-283 | 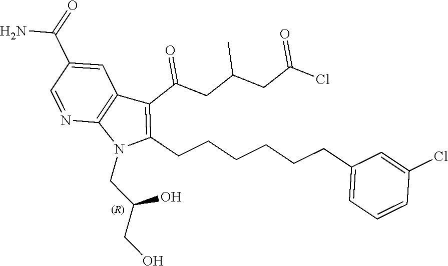 | 558.2 | ¹H NMR (400 MHz, DMSO-d₆) δ 12.09 (br., 1H), 8.78 (d, J = 2.0 Hz, 1H), 8.71 (d, J = 1.6 Hz, 1H), 8.22 (s, 1H), 7.50 (s, 1H), 7.31-7.16 (m, 4H), 5.00 (d, J = 2.4 Hz, 1H), 4.86-4.83 (m, 1H), 4.51 (d, J = 12.0 Hz, 1H), 4.16-4.11 (m, 1H), 3.95-3.89 (m, 1H), 346-3.39 (m, 3H), 3.26-3.19 (m, 1H), 3.11-3.04 (m, 1H), 2.98-2.90 (m, 1H), 2.58 (t, J = 7.6 Hz, 2H), 2.54-2.53 (m, 1H), 2.45-2.39 (m, 1H), 2.21-2.14 (m, 1H), 1.62-1.55 (m, 4H), 1.48-1.41 (m, 2H), 1.38-1.30 (m, 2H), 0.99 (dd, J = 6.4, 3.2 Hz, 3H). |
| I-285 | 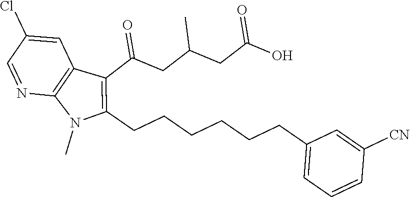 [SMILES: CC(CC(=O)O)CC(=O)c1c(CCCCCCc2cccc(C#N)c2)n(C)c2ncc(Cl)cc12] | 480.3 | ¹H NMR (400 MHz, DMSO-d₆) δ 12.12 (br., 1H), 8.37 (d, J = 2.4 Hz, 1H), 8.33 (d, J = 2.0 Hz, 1H), 7.68 (s, 1H), 7.63 (d, J = 7.6 Hz, 1H), 7.56 (d, J = 7.6 Hz, 1H), 7.48 (t, J = 7.6 Hz, 1H), 3.80 (s, 3H), 3.18 (t, J = 7.6 Hz, 2H), 3.04 (dd, J = 16.8, 6.4 Hz, 1H), 2.86 (d, J = 16.8, 7.6 Hz, 1H), 2.64 (t, J = 7.6 Hz, 2H), 2.50-2.49 (m, 1H), 2.39 (dd, J = 15.2, 6.0 Hz, 1H), 2.18 (dd, J = 15.2, 8.0 Hz, 1H), 1.64-1.58 (m, 4H), 1.84-1.43 (m, 2H), 1.38-1.32 (m, 2H), 0.96 (d, J = 6.4 Hz, 3H). |
| I-212 | 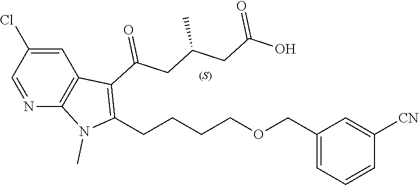 | 482.2 | ¹H NMR (400 MHz, DMSO-d₆) δ 12.08 (s, 1H), 8.37 (d, J = 2.0 Hz, 1H), 8.33 (d, J = 2.0 Hz, 1H), 7.76-7.74 (m, 2H), 7.67 (d, J = 7.2 Hz, 1H), 7.56 (t, J = 7.6 Hz, 1H), 4.52 (s, 2H), 3.81 (s, 3H), 3.52 (t, J = 4.8 Hz, 2H), 3.23 (t, J = 7.2 Hz, 2H), 3.05 (dd, J = 16.4, 5.6 Hz, 1H), 2.87 (dd, J = 16.4, 7.6 Hz, 1H), 2.50-2.48 (m, 1H), 2.39 (dd, J = 15.6, 5.6 Hz, 1H), 2.17 (dd, J = 14.8, 7.6 Hz, 1H), 1.70-1.68 (m, 4H), 0.96 (d, J = 6.4 Hz, 3H). |
| I-213 | 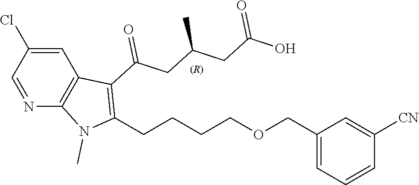 | 482.1 | ¹H NMR (400 MHz, DMSO-d₆) δ 12.07 (s, 1H), 8.37 (d, J = 2.0 Hz, 1H), 8.33 (d, J = 2.0 Hz, 1H), 7.76-7.74 (m, 2H), 7.67 (d, J = 8.0 Hz, 1H), 7.56 (t, J = 7.2 Hz, 1H), 4.52 (s, 2H), 3.81 (s, 3H), 3.52 (t, J = 5.6 Hz, 2H), 3.23 (t, J = 7.6 Hz, 2H), 3.05 (dd, J = 16.4, 5.6 Hz, 1H), 2.87 (dd, J = 16.8, 8.0 Hz, 1H), 2.50-2.48 (m, 1H), 2.39 (dd, J = 15.6, 5.6 Hz, 1H), 2.17 (dd, J = 15.6, 8.0 Hz, 1H), 1.70-1.68 (m, 4H), 0.96 (d, J = 6.4 Hz, 3H). |
| I-215 | 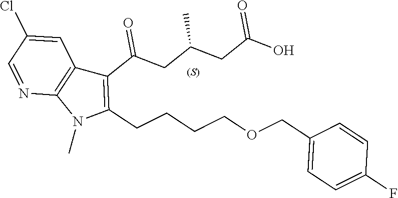 | 475.1 | ¹H NMR (400 MHz, DMSO-d₆) δ 12.11 (s, 1H), 8.37 (d, J = 2.4 Hz, 1H), 8.33 (d, J = 2.0 Hz, 1H), 7.37-7.34 (m, 2H), 7.15 (t, J = 9.2 Hz, 2H), 4.44 (s, 2H), 3.80 (s, 3H), 3.49 (t, J = 5.2 Hz, 2H), 3.22 (t, J = 6.8 Hz, 2H), 3.04 (dd, J = 16.8, 5.6 Hz, 1H), 2.87 (dd, J = 16.4, 7.6 Hz, 1H), 2.48- 2.45 (m, 1H), 2.39 (dd, J = 15.6, 6.0 Hz, 1H), 2.17 (dd, J = 15.2, 7.6 Hz, 1H), 1.69- 1.67 (m, 4H), 0.96 (d, J = 6.4 Hz, 3H). |
| I-216 | 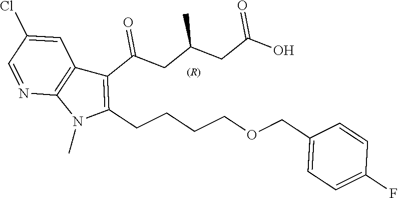 | 475.1 | ¹H NMR (400 MHz, DMSO-d₆) δ 12.14 (s, 1H), 8.37 (d, J = 2.0 Hz, 1H), 8.33 (d, J = 2.0 Hz, 1H), 7.37-7.34 (m, 2H), 7.15 (t, J = 8.8 Hz, 2H), 4.44 (s, 2H), 3.80 (s, 3H), 3.49 (t, J = 4.8 Hz, 2H), 3.22 (t, J = 6.8 Hz, 2H), 3.04 (dd, J = 16.8, 5.6 Hz, 1H), 2.86 (dd, J = 16.4, 7.6 Hz, 1H), 2.48- 2.45 (m, 1H), 2.39 (dd, J = 16.0, 6.0 Hz, 1H), 2.17 (dd, J = 15.6, 8.0 Hz, 1H), 1.69- 1.67 (m, 4H), 0.96 (d, J = 6.4 Hz, 3H). |
| I-220 | 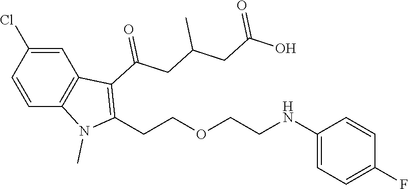 [SMILES: CC(CC(=O)O)CC(=O)c1c(CCOCCNc2ccc(F)cc2)n(C)c2ccc(Cl)cc12] | 475.3 | ¹H NMR (400 MHz, CDCl₃) δ 7.87 (s, 1H), 7.27-7.26 (m, 2H), 6.82 (t, J = 8.8 Hz, 2H), 6.35-6.32 (m, 2H), 3.83 (t, J = 5.6 Hz, 2H), 3.75 (s, 3H), 3.61 (t, J = 4.8 Hz, 2H), 3.51-3.47 (q, 2H), 3.14 (t, J = 5.2 Hz, 2H), 3.08-2.96 (m, 2H), 2.75-2.67 (m, 1H), 2.50 (dd, J = 14.8, 6.4 Hz, 1H), 2.36 (dd, J = 14.8, 6.4Hz, 1H), 1.16 (d, J = 6.8 Hz, 3H). |
| I-231 | 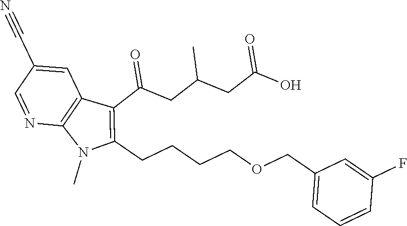 [SMILES: CC(CC(=O)O)CC(=O)c1c(CCCCOCc2cccc(F)c2)n(C)c2ncc(C#N)cc12] | 466.2 | ¹H NMR (400 MHz, DMSO-d₆) δ 12.11 (br., 1H), 8.83 (d, J = 1.6 Hz, 1H), 8.73 (d, J = 1.6 Hz, 1H), 7.40-7.35 (m, 1H), 7.16- 7.07 (m, 3H), 4.48 (s, 2H), 3.85 (s, 3H), 3.51 (t, J = 4.8 Hz, 2H), 3.26 (t, J = 6.8 Hz, 2H), 3.10 (dd, J = 16.8, 5.6 Hz, 1H), 2.91 (dd, J = 16.8, 8.0 Hz, 1H), 2.51-2.49 (m, 1H), 2.41 (dd, J = 15.6, 6.0 Hz, 1H), 2.18 (dd, J = 15.2, 7.6 Hz, 1H), 1.71-1.69 (m, 4H), 0.96 (d, J = 6.4 Hz, 3H). |
| I-293 | 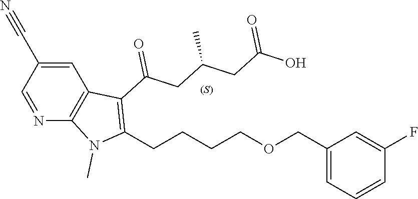 | 466.3 | ¹H NMR (400 MHz, DMSO-d₆) δ 12.13 (br., 1H), 8.83 (d, J = 2.0 Hz, 1H), 8.73 (d, J = 1.6 Hz, 1H), 7.40-7.35 (m, 1H), 7.16- 7.07 (m, 3H), 4.48 (s, 2H), 3.85 (s, 3H), 3.51 (t, J = 5.6 Hz, 2H), 3.25 (t, J = 7.2 Hz, 2H), 3.10 (dd, J = 16.8, 5.6 Hz, 1H), 2.91 (dd, J = 16.8, 8.0 Hz, 1H), 2.51-2.48 (m, 1H), 2.41 (dd, J = 15.6, 6.0 Hz, 1H), 2.18 (dd, J = 15.6, 8.0 Hz, 1H), 1.71-1.69 (m, 4H), 0.96 (d, J = 6.4 Hz, 3H). |
| I-294 | 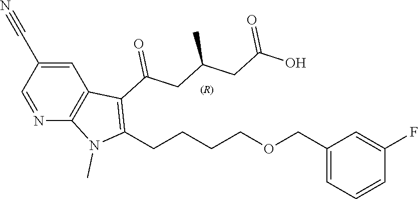 | 466.2 | ¹H NMR (400 MHz, DMSO-d₆) δ 12.11 (br., 1H), 8.83 (d, J = 1.6 Hz, 1H), 8.73 (d, J = 2.0 Hz, 1H), 7.40-7.35 (m, 1H), 7.16- 7.07 (m, 3H), 4.48 (s, 2H), 3.85 (s, 3H), 3.51 (t, J = 5.6 Hz, 2H), 3.25 (t, J = 7.6 Hz, 2H), 3.10 (dd, J = 16.8, 5.6 Hz, 1H), 2.92 (dd, J = 16.8, 8.0 Hz, 1H), 2.51-2.48 (m, 1H), 2.41 (dd, J = 15.2, 5.6 Hz, 1H), 2.18 (dd, J = 15.6, 8.0 Hz, 1H), 1.71-1.69 (m, 4H), 0.96 (d, J = 6.4 Hz, 3H). |
| I-295 | 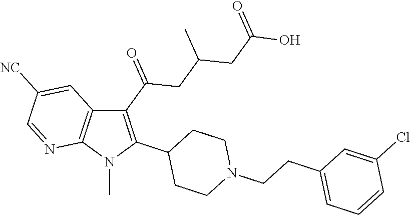 [SMILES: CC(CC(=O)O)CC(=O)c1c(C2CCN(CCc3cccc(Cl)c3)CC2)n(C)c2ncc(C#N)cc12] | 507.3 | ¹H NMR (400 MHz, DMSO-d₆) δ 8.81 (d, J = 2.0 Hz, 1H), 8.74 (d, J = 2.0 Hz, 1H), 7.35-7.31 (m, 2H), 7.27-7.23 (m, 2H), 3.98 (s, 3H), 3.68 (t, J = 13.2 Hz, 1H), 3.16-3.07 (m, 3H), 2.93 (dd, J = 16.8, 7.6 Hz, 1H), 2.82-2.78 (m, 2H), 2.60-2.56 (m, 2H), 2.53-2.50 (m, 1H), 2.44-2.39 (m, 1H), 2.33-2.24 (m, 2H), 2.21-2.09 (m, 3H), 1.66 (d, J = 11.6 Hz, 2H), 0.97 (d, J = 6.4 Hz, 3H). |
| I-292 | 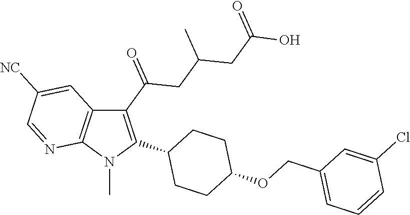 [SMILES: CC(CC(=O)O)CC(=O)c1c2cc(C#N)cnc2n(C)c1[C@H]1CC[C@@H](OCc2cccc(Cl)c2)CC1] | 508.2 | ¹H NMR (400 MHz, DMSO-d₆) δ 12.16 (br., 1H), 8.79 (d, J = 1.6 Hz, 1H), 8.73 (d, J = 1.2 Hz, 1H), 7.52 (s, 1H), 7.46-7.38 (m, 2H), 7.37-7.33 (m, 1H), 4.56 (s, 2H), 3.98 (s, 3H), 3.95-3.82 (m, 1H), 3.76 (s, 1H), 3.13 (dd, J = 16.8, 5.6 Hz, 1H), 2.92-2.85 (m, 1H), 2.70-2.63 (m, 1H), 2.40-2.30 (m, 3H), 2.11-2.03 (m, 3H), 1.63-1.47 (m, 4H), 0.96 (d, J = 6.4 Hz, 3H). |
| I-288 | 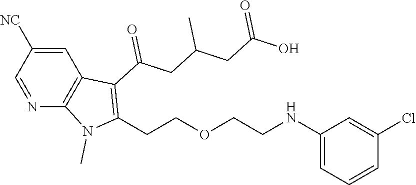 [SMILES: CC(CC(=O)O)CC(=O)c1c(CCOCCNc2cccc(Cl)c2)n(C)c2ncc(C#N)cc12] | 483.2 | ¹H NMR (400 MHz, DMSO-d₆) δ 12.08 (br., 1H), 8.83 (d, J = 1.6 Hz, 1H), 8.73 (d, J = 1.6 Hz, 1H), 7.02-6.98 (m, 1H), 6.49- 6.47 (m, 2H), 6.43 (dd, J = 8.4, 1.2 Hz, 1H), 5.80 (s, 1H), 3.88 (s, 3 H), 3.77 (t, J = 6.4 Hz, 2H), 3.53-3.47 (m, 4H), 3.14- 3.09 (m, 3H), 2.94 (dd, J = 16.8, 7.6 Hz, 1H), 2.54-2.50 (m, 1H), 2.42 (dd, J = 15.6, 5.6 Hz, 1H), 2.18 (dd, J = 15.6, 8.0 Hz, 1H), 0.97 (d, J = 6.4 Hz, 3H). |
| I-296 | 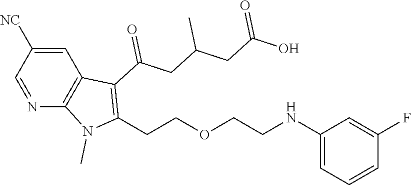 [SMILES: CC(CC(=O)O)CC(=O)c1c(CCOCCNc2cccc(F)c2)n(C)c2ncc(C#N)cc12] | 467.1 | ¹H NMR (400 MHz, DMSO-d₆) δ 12.08 (s, 1H), 8.84 (d, J = 2.0 Hz, 1H), 8.73 (d, J = 1.6 Hz, 1H), 7.03-6.98 (m, 1H), 6.32- 6.30 (m, 1H), 6.26-6.22 (m, 2H), 5.81 (br., 1H), 3.88 (s, 3 H), 3.77 (t, J = 6.4 Hz, 2H), 3.53-3.47 (m, 4H), 3.17-3.08 (m, 3H), 2.94 (dd, J = 17.2, 7.6 Hz, 1H), 2.54-2.50 (m, 1H), 2.42 (dd, J = 15.6, 5.6 Hz, 1H), 2.18 (dd, J = 15.6, 8.0 Hz, 1H), 0.97 (d, J = 6.4 Hz, 3H). |
| I-290 | 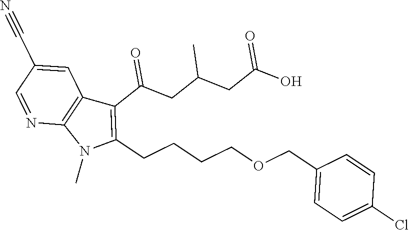 [SMILES: CC(CC(=O)O)CC(=O)c1c(CCCCOCc2ccc(Cl)cc2)n(C)c2ncc(C#N)cc12] | 482.0 | ¹H NMR (400 MHz, DMSO-d₆) δ 12.10 (br., 1H), 8.83 (d, J = 2.0 Hz, 1H), 8.73 (d, J = 1.6 Hz, 1H), 7.40-7.32 (m, 4H), 4.45 (s, 2H), 3.85 (s, 3H), 3.50 (t, J = 5.2 Hz, 2H), 3.24 (t, J = 7.2 Hz, 2H), 3.10 (dd, J = 16.8, 5.6 Hz, 1H), 2.92 (dd, J = 16.8, 7.6 Hz, 1H), 2.50-2.48 (m, 1H), 2.41 (dd, J = 15.2, 5.6 Hz, 1H), 2.18 (dd, J = 15.6, 8.0 Hz, 1H), 1.70-1.69 (m, 4H), 0.96 (d, J = 6.8 Hz, 3H). |
| I-297 | 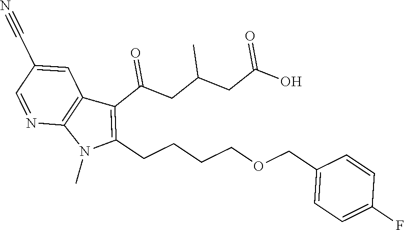 [SMILES: CC(CC(=O)O)CC(=O)c1c(CCCCOCc2ccc(F)cc2)n(C)c2ncc(C#N)cc12] | 466.3 | ¹H NMR (400 MHz, DMSO-d₆) δ 12.08 (br., 1H), 8.83 (d, J = 1.6 Hz, 1H), 8.73 (d, J = 1.6 Hz, 1H), 7.37-7.34 (m, 2H), 7.15 (t, J = 8.8 Hz, 2H), 4.44 (s, 2H), 3.84 (s, 3H), 3.49 (t, J = 5.2 Hz, 2H), 3.24 (t, J = 6.8 Hz, 2H), 3.10 (dd, J = 16.8, 5.6 Hz, 1H), 2.92 (dd, J = 16.8, 7.6 Hz, 1H), 2.50- 2.48 (m, 1H), 2.41 (dd, J = 15.6, 6.0 Hz, 1H), 2.18 (dd, J = 15.2, 8.0 Hz, 1H), 1.69- 1.68 (m, 4H), 0.96 (d, J = 6.8 Hz, 3H). |
| I-291 | 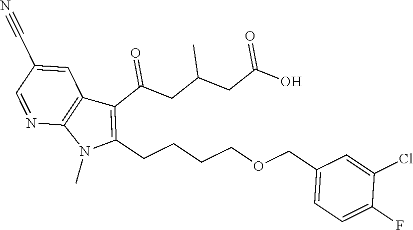 [SMILES: CC(CC(=O)O)CC(=O)c1c(CCCCOCc2ccc(F)c(Cl)c2)n(C)c2ncc(C#N)cc12] | 500.2 | ¹H NMR (400 MHz, DMSO-d₆) δ 12.11 (br., 1H), 8.83 (s, 1H), 8.73 (s, 1H), 7.51 (d, J = 7.2 Hz, 1H), 7.40-7.33 (m, 2H), 4.45 (s, 2H), 3.85 (s, 3H), 3.50 (t, J = 5.2 Hz, 2H), 3.25 (t, J = 6.8 Hz, 2H), 3.10 (dd, J = 16.8, 5.6 Hz, 1H), 2.92 (dd, J = 16.8, 8.0 Hz, 1H), 2.54-2.48 (m, 1H), 2.41 (dd, J = 15.6, 5.6 Hz, 1H), 2.18 (dd, J = 15.2, 7.6, Hz, 1H), 1.70-1.69 (m, 4H), 0.96 (d, J = 6.4 Hz, 3H). |
| I-160 | 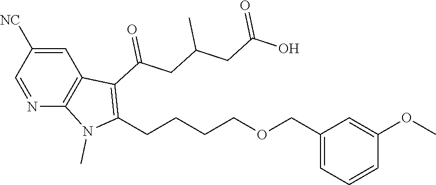 [SMILES: COc1cccc(COCCCCc2c(C(=O)CC(C)CC(=O)O)c3cc(C#N)cnc3n2C)c1] | 478.2 | ¹H NMR (400 MHz, DMSO-d₆) δ 12.09 (br., 1H), 8.83 (d, J = 2.0 Hz, 1H), 8.73 (d, J = 1.6 Hz, 1H), 7.24 (t, J = 8.0 Hz, 1H), 6.89-6.81 (m, 3H), 4.43 (s, 2H), 3.85 (s, 3H), 3.73 (s, 3H), 3.49 (t, J = 5.2 Hz, 2H), 3.25 (t, J = 6.8 Hz, 2H), 3.10 (dd, J = 17.2, 5.6 Hz, 1H), 2.91 (dd, J = 16.8, 7.6 Hz, 1H), 2.50-2.47 (m, 1H), 2.41 (dd, J = 15.6, 6.0 Hz, 1H), 2.18 (dd, J = 15.6, 8.0 Hz, 1H), 1.70-1.68 (m, 4H), 0.96 (d, J = 6.4 Hz, 3H). |
| I-298 | 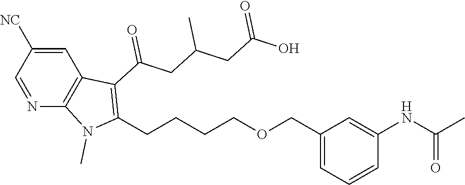 [SMILES: CC(=O)Nc1cccc(COCCCCc2c(C(=O)CC(C)CC(=O)O)c3cc(C#N)cnc3n2C)c1] | 505.3 | ¹H NMR (400 MHz, DMSO-d₆) δ 12.11 (br., 1H), 9.91 (s, 1H), 8.83 (d, J = 2.0 Hz, 1H), 8.73 (d, J = 2.0 Hz, 1H), 7.55 (s, 1H), 7.47 (d, J = 8.0 Hz, 1H), 7.23 (t, J = 8.0 Hz, 1H), 6.96 (d, J = 7.6 Hz, 1H), 4.42 (s, 2H), 3.84 (s, 3H), 3.49 (t, J = 5.2 Hz, 2H), 3.26 (t, J = 6.8 Hz, 2H), 3.10 (dd, J = 17.2, 5.6 Hz, 1H), 2.91 (dd, J = 16.4, 7.6 Hz, 1H), 2.49-2.47 (m, 1H), 2.41 (dd, J = 15.2, 6.0 Hz, 1H), 2.18 (dd, J = 15.6, 8.0 Hz, 1H), 2.01 (s, 3H), 1.70-1.69 (m, 4H), 0.96 (d, J = 6.8 Hz, 3H). |
| I-299 | 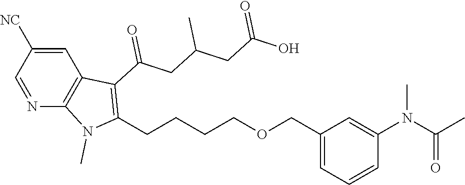 [SMILES: CC(=O)N(C)c1cccc(COCCCCc2c(C(=O)CC(C)CC(=O)O)c3cc(C#N)cnc3n2C)c1] | 519.3 | ¹H NMR (400 MHz, DMSO-d₆) δ 12.11 (br., 1H), 8.83 (d, J = 1.6 Hz, 1H), 8.73 (d, J = 1.6 Hz, 1H), 7.41 (t, J = 7.2 Hz, 1H), 7.30-7.22 (m, 3H), 4.49 (s, 2 H), 3.84 (s, 3 H), 3.52 (t, J = 5.2 Hz, 2H), 3.25 (t, J = 6.8 Hz, 2H), 3.13-3.07 (m, 4H), 2.92 (dd, J = 16.8, 8.0 Hz, 1H), 2.51-2.45 (m, 1H), 2.41 (dd, J = 15.2, 5.6 Hz, 1H), 2.18 (dd, J = 15.6, 8.0 Hz, 1H), 1.37-1.69 (m, 7H), 0.96 (d, J = 6.4 Hz, 3H). |
| I-300 | 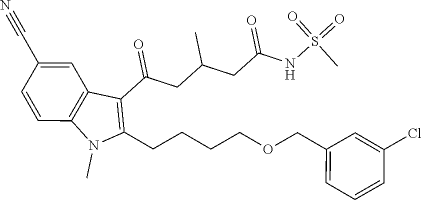 [SMILES: CC(CC(=O)NS(C)(=O)=O)CC(=O)c1c(CCCCOCc2cccc(Cl)c2)n(C)c2ccc(C#N)cc12] | 558.1 | ¹H NMR (400 MHz, DMSO-d₆) δ 8.42 (s, 1H), 7.76 (d, J = 8.4 Hz, 1H), 7.62 (dd, J = 8.4, 1.2 Hz, 1H), 7.39-7.27 (m, 4H), 4.47 (s, 2H), 3.81 (s, 3H), 3.51 (t, J = 6.0 Hz, 2H), 3.20 (t, J = 7.6 Hz, 2H), 3.09 (s, 3H), 3.06 (dd, J = 16.8, 5.6 Hz, 1H), 2.87 (dd, J = 16.4, 8.0 Hz, 1H), 2.56-2.51 (m, 1H), 2.33 (dd, J = 14.8, 6.0 Hz, 1H), 2.15 (dd, J = 14.8, 8.0 Hz, 1H), 1.72-1.66 (m, 4H), 0.94 (d, J = 6.4 Hz, 3H). |

### Example 3: Synthesis of Compound I-44

### Synthetic Scheme for Compound 1-44

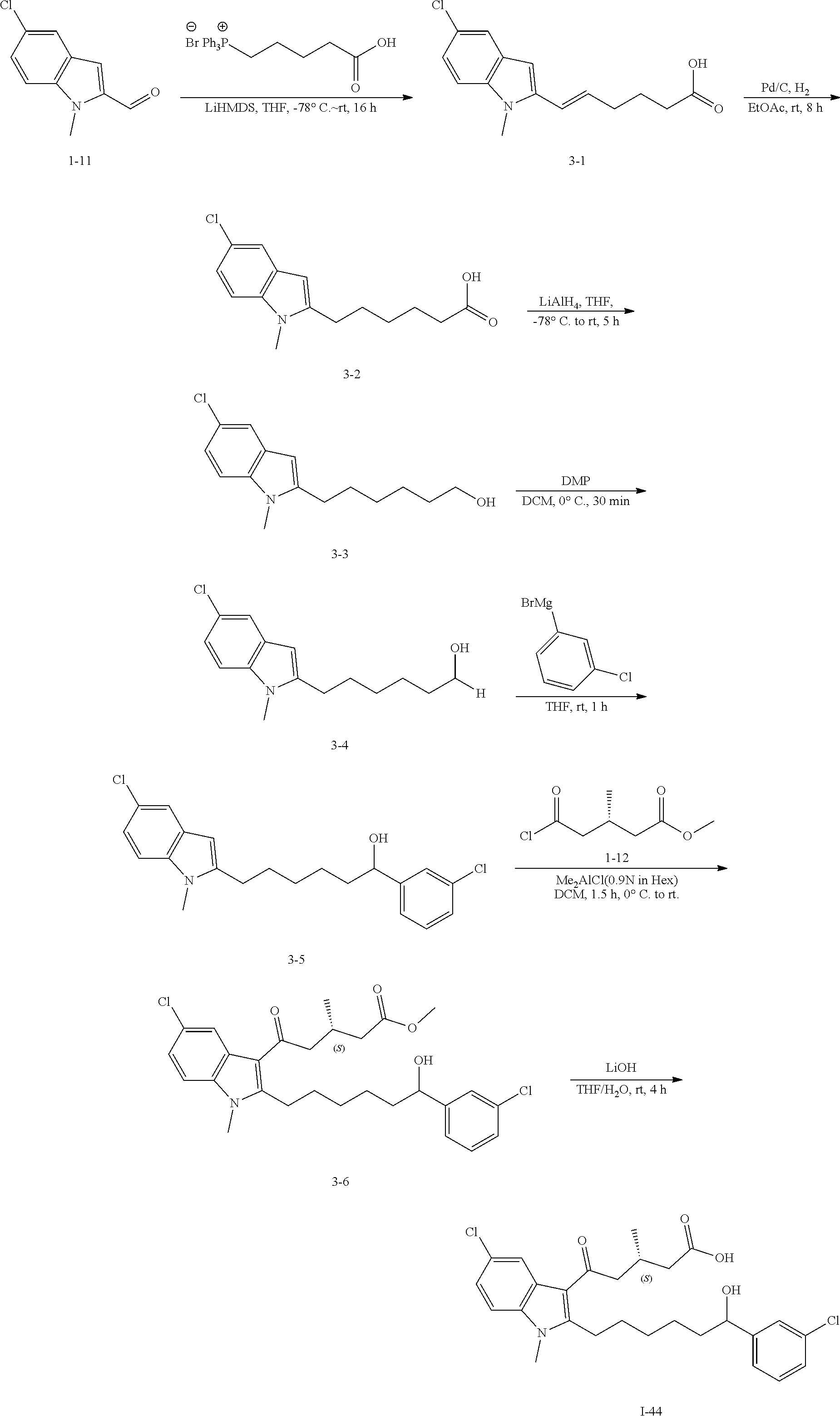

### 3.1 The Synthesis of Intermediate 3-1

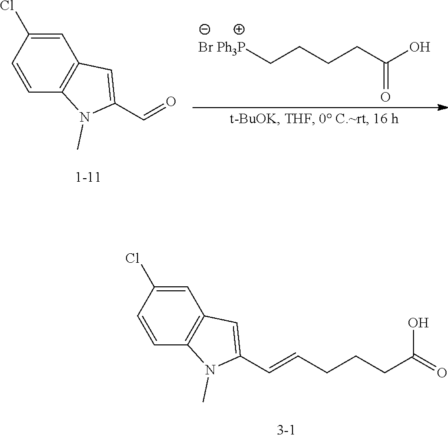

To a suspension of (4-carboxybutyl)triphenylphosphonium bromide (48.6 g, 110 mmol) in THF (800 mL) was added t-BuOK (135.7 g, 275 mmol) at 0° C. The mixture was stirred for 30 min and 1-11 (21.2 g, 110.0 mmol) in THF (200 ml) was added dropwise. The reaction mixture was allowed to warm to room temperature and stirred for 16 h. Saturated NH₄Cl solution was added at 0° C. and the mixture was acidified with HCl (1.0 M) to pH˜3, extracted with EtOAc, the combined organic layer was washed with brine, dried over anhydrous Na₂SO₄ and evaporated under reduced pressure. The crude was purified by silica gel chromatography (5% MeOH/DCM) to afford 3-1 (30.0 g, 98%) as yellow oil. LC-MS m/z: 278.0 [M+H]⁺.

### 3.2 The Synthesis of Intermediate 3-2

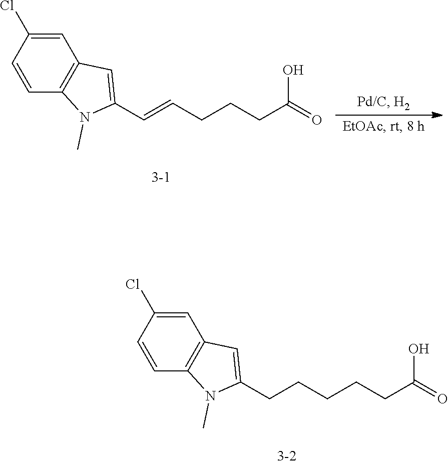 [SMILES: Cn1c(/C=C/CCCC(=O)O)cc2cc(Cl)ccc21 ; "EtOAc, rt, 8 h" ; "Pd/C, H2" ; Cn1c(CCCCCC(=O)O)cc2cc(Cl)ccc21]

To a stirred solution of 3-1 (30.0 g, 108.3 mmol) in EtOAc (500 mL) was added 10% Pd/C (6.0 g) under H₂ atmosphere (1.0 atm). The reaction mixture was stirred at room temperature for 8 h until the reaction was complete (by LCMS) and then filtered. The residue was washed with EtOAc, and the combined filtrate was concentrated under reduced pressure to afford 3-2 (28.0 g, 93%) as colorless oil. LC-MS m/z: 279.7 [M+H]⁺.

### 3.3 The Synthesis of Intermediate 3-3

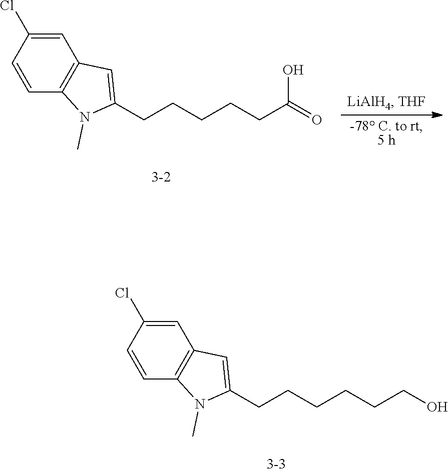

To a stirred solution of 3-2 (28.0 g, 100.4 mmol) in THF (500 mL) was added LiAlH₄ (7.63 g, 200.8 mmol) slowly at −78° C. The reaction mixture was allowed to warm to room temperature and stirred for 5 h until the reaction was complete (by LCMS). Water (8 mL), NaOH (a.q. 15%, 8 mL) and water (24 mL) was added dropwise at 0° C. successively to quench the reaction. The mixture was filtered and the filtrate was dried over anhydrous Na₂SO₄ then evaporated under reduced pressure. The crude product was purified by silica gel chromatography (30% EtOAc/PE) to afford 3-3 (17.5 g, 66%) as a yellow solid. LC-MS m/z: 266.0 [M+H]⁺.

### 3.4 The Synthesis of Intermediate 3-4

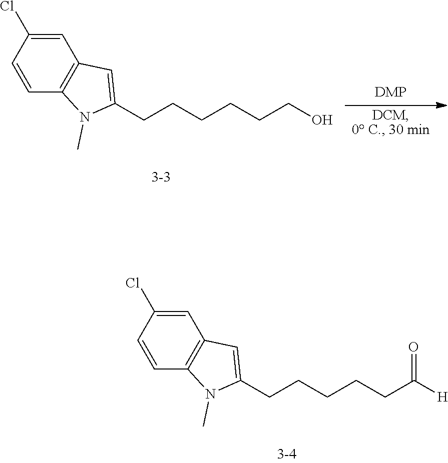

To a solution of 3-3 (17.5 g, 66.0 mmol) in DCM (500 mL) was added DMP (33.6 g, 79.2 mmol) at 0° C. The mixture was stirred for 30 min at 0° C. Saturated NaHCO₃ (aq., 50 mL) was added to the mixture and it was extracted with DCM (300 mL×3). The combined organic layer was washed with brine (200 mL×2), dried over anhydrous Na₂SO₄, filtered and concentrated in vacuo. The crude product was purified by silica gel chromatography (20% EtOAc/PE) to afford 3-4 (5.0 g, 29%) as a yellow solid. LC-MS m/z: 264.1 [M+H]⁺.

### 3.5 The Synthesis of Intermediate 3-5

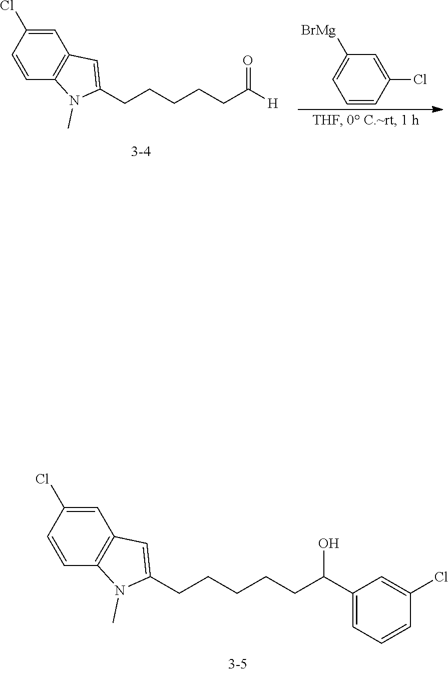

To a solution of 3-4 (5.0 g, 19.0 mmol) in THF (200 mL) was added (3-chlorophenyl)magnesium bromide (1 M in THF, 23.0 mL, 23.0 mmol) at 0° C. The mixture was stirred at room temperature for 1 h. Water (20 mL) was added and the mixture was extracted with EtOAc (50 mL×3). The combined organic layer was dried over anhydrous Na₂SO₄, filtered and concentrated in vacuo. The crude product was purified by silica gel chromatography (30% EtOAc/PE) to afford 3-5 (4.5 g, 63%) as a white solid. LC-MS m/z: 376.0 [M+H]⁺.

### 3.6 The Synthesis of Intermediate 3-6

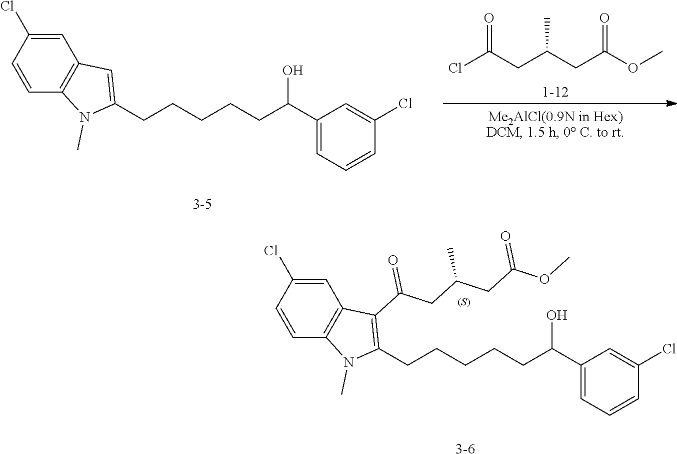

To a stirred solution of 3-5 (300 mg, 0.8 mmol) in dichloromethane (15 mL) was added Me₂AlCl (0.9 M in hexane, 2.0 mL, 2.0 mmol) at 0° C. After 30 min, 1-12 (284.8 mg, 1.6 mmol) in CH₂Cl₂ (2 mL) was added dropwise and the reaction mixture was stirred at room temperature for another 1 h. The reaction was quenched by adding water and extracted with EtOAc. The organic layer was combined, washed with brine and dried over Na₂SO₄. The solvent was evaporated under reduced pressure and the crude was purified by silica gel chromatography (30% EtOAc/Hex) to afford 3-6 (180 mg, 44%) as colorless oil.

### 3.7 The Synthesis of Compound I-44

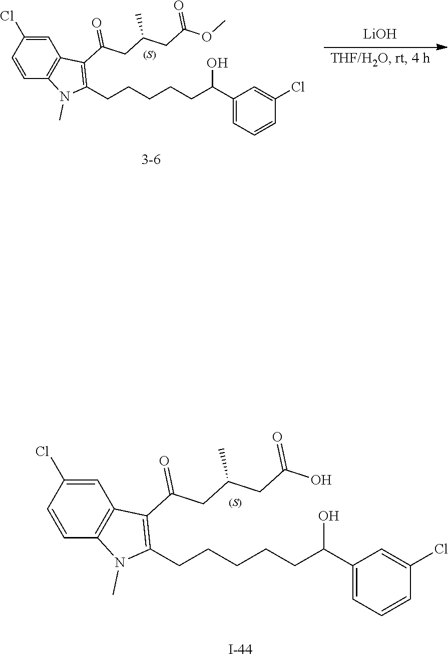

To a mixture of 3-6 (180 mg, 0.35 mmol) in THF/H₂O (10 mL/2 mL) was added lithium hydroxide (147.0 mg, 3.5 mmol), the reaction mixture was stirred at room temperature for 4 h until the reaction was complete (by LCMS). Saturated NH₄Cl solution was added at 0° C. and the mixture was acidified with HCl (1.0 M) to pH -3.0 then extracted with EtOAc, the combined organic layer was washed with brine, dried over anhydrous Na₂SO₄ and evaporated under reduced pressure. The crude was purified by Prep-HPLC to afford I-44 (73.9 mg, 42%) as a white solid. ¹H NMR (400 MHz, DMSO-d₆) δ 12.11 (br, 1H), 7.95 (d, J=1.2 Hz, 1H), 7.58 (d, J=8.4 Hz, 1H), 7.36-7.31 (m, 2H), 7.27-7.23 (m, 3H), 5.32-5.21 (m, 1H), 4.53 (t, J=6.0 Hz, 1H), 3.74 (s, 3H), 3.11 (t, J=8.0 Hz, 2H), 2.98 (dd, J=16.4, 5.6 Hz, 1H), 2.84 (dd, J=16.4, 7.6 Hz, 1H), 2.48-2.45 (m, 1H), 2.38 (dd, J=15.6, 5.6 Hz, 1H), 2.17 (dd, J=15.2, 8.0 Hz, 1H), 1.59-1.54 (m, 4H), 1.41-1.23 (m, 4H), 0.96 (d, J=5.2 Hz, 3H). LC-MS m/z: 504.0 [M+H]⁺.

Additional exemplary compounds were prepared following methods substantially similar to the method described above and herein. Data for these compounds are provided in Tables 4 and 4a below.

TABLE 4
Characterization Data for Additional Exemplary Compounds

| Compound | | | |
|---|---|---|---|
| No. | Chemical Structure | M + 1 | ¹H NMR (400 MHz) |
| I-3 | 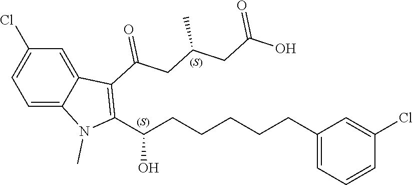 | 503.9 | ¹H NMR (400 MHz, CDCl₃) δ 7.82 (s, 1H), 7.17-7.10 (m, 5H), 6.99-6.98 (m, 1H), 4.96 (s, 1H), 3.73 (s, 3H), 3.12- 2.95 (m, 2H), 2.74-2.73 (m, 1H), 2.52- 2.48 (m, 3H), 2.34-2.30 (m, 1H), 1.96- 1.92 (m, 1H), 1.58-1.55 (m, 4H), 1.30- 1.24 (m, 4H), 1.07(s, 3H). |
| I-4 | 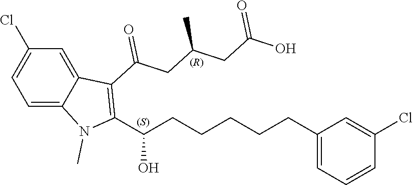 | 503.9 | ¹H NMR (400 MHz, CDCl₃) δ 7.85 (s, 1H), 7.30-7.27 (m, 2H), 7.18-7.11 (m, 3H), 7.00 (d, J = 6.4 Hz, 1H) 4.96-4.95 (m, 1H), 3.75 (s, 3H), 3.12-3.10 (m, 1H), 3.03-2.98 (m, 1H), 2.73-2.72 (m, 1H), 2.54 (t, J = 7.6 Hz, 3H), 2.38-2.28 (m, 1H), 1.96-1.91 (m, 1H), 1.60-1.53 (m, 4H), 1.36-1.21 (m, 4H), 1.14 (d, J = 6.8 Hz, 3H). |
| I-5 | 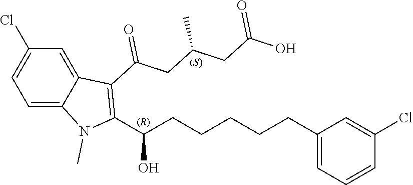 | 503.9 | ¹H NMR (400 MHz, CDCl₃) δ 7.84 (s, 1H), 7.29-7.27 (m, 2H), 7.18-7.10 (m, 3H), 7.00-6.98 (m, 1H) 4.97 (s, 1H), 3.75 (s, 3H), 3.12-2.97 (m, 2H), 2.72- 2.71 (m, 1H), 2.55-2.51 (m, 3H), 2.36- 2.30 (m, 1H), 2.00-1.92 (m, 1H), 1.60- 1.56 (m, 4H), 1.32-1.20 (m, 4H), 1.13(d, J = 5.6Hz, 3H). |
| I-6 | 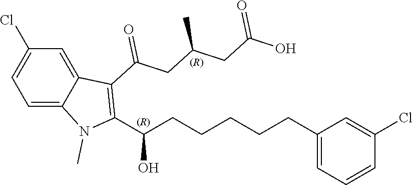 | 503.9 | ¹H NMR (400 MHz, CDCl₃) δ 7.78 (s, 1H), 7.21-7.10 (m, 5H), 6.99-6.97 (m, 1H), 5.01 (s, 1H), 3.71 (s, 3H), 3.04- 2.88 (m, 2H), 2.70-2.64 (m, 1H), 2.49- 2.37 (m, 3H), 2.29-2.21 (m, 1H), 1.92- 1.90 (m, 1H), 1.57-1.51 (m, 4H), 1.32- 1.19 (m, 4H), 1.01-0.97 (m, 3H). |
| I-45 | 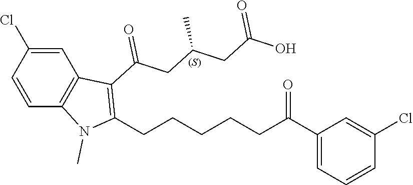 | 502.0 | ¹H NMR (400 MHz, DMSO-d₆) δ 12.08 (s, 1H), 7.95-7.92 (m, 3H), 7.70 (d, J = 7.6 Hz, 1H), 7.61-7.54 (m, 2H), 7.25 (d, J = 8.8 Hz, 1H), 3.77 (s, 3H), 3.15 (t, J = 7.6 Hz, 2H), 3.08 (t, J = 6.8 Hz, 2H), 2.99 (dd, J = 16.4, 5.6 Hz, 1H), 2.85 (dd, J = 16.4, 7.6 Hz, 1H), 2.48- 2.44 (m, 1H), 2.39 (dd, J = 15.2, 5.2 Hz, 1H), 2.17 (dd, J = 15.6, 8.0 Hz, 1H), 1.70-1.62 (m, 4H), 1.49-1.47 (m, 2H), 0.97 (d, J = 6.0 Hz, 3H). |
| I-47 | 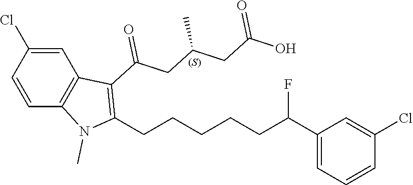 | 505.9 | ¹H NMR (400 MHz, DMSO-d₆) δ 12.03 (br, 1H), 7.94 (d, J = 2.0 Hz, 1H), 7.57 (d, J = 8.8 Hz, 1H), 7.43-7.37 (m, 3H), 7.33 (d, J = 6.4 Hz, 1H), 7.23 (dd, J = 8.8, 2.0 Hz, 1H), 5.55 (dt, J = 48.0, 4.8 Hz, 1H), 3.74 (s, 3H), 3.12 (t, J = 8.0 Hz, 2H), 2.98 (dd, J = 16.4, 6.0 Hz, 1H), 2.83 (dd, J = 16.4, 7.6 Hz, 1H), 2.48- 2.46 (m, 1H), 2.38 (dd, J = 15.6, 5.6 Hz, 1H), 2.16 (dd, J = 15.2, 8.0 Hz, 1H), 1.97-1.76 (m, 2H), 1.57-1.55 (m, 2H), 1.45-1.33 (m, 3H), 0.96 (d, J = 6.4 Hz, 3H). |
| I-85 | 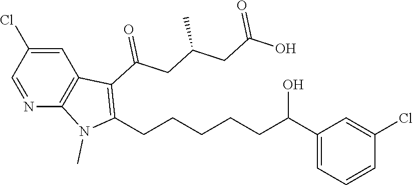 [SMILES: C[C@H](CC(=O)O)CC(=O)c1c(CCCCCC(O)c2cccc(Cl)c2)n(C)c2ncc(Cl)cc12] | 504.9 | ¹H NMR (400 MHz, CDCl₃) δ 8.18 (d, J = 2.0 Hz, 1H), 8.13 (d, J = 1.6 Hz, 1H), 7.27 (s, 1H), 7.19-7.12 (m, 3H), 4.62 (t, J = 6.0 Hz, 1H), 3.75 (s, 3H), 3.16-3.06 (m, 2H), 3.00-2.94 (m, 1H), 2.84-2.78 (m, 1H), 2.68-2.63 (m, 1H), 2.48-2.43 (m, 1H), 2.32 (dd, J = 15.2 Hz, 6.8 Hz, 1H), 1.74-1.54 (m, 4H), 1.45-1.36 (m, 4H), 1.06 (d, J = 6.8 Hz, 3H). |
| I-173 | 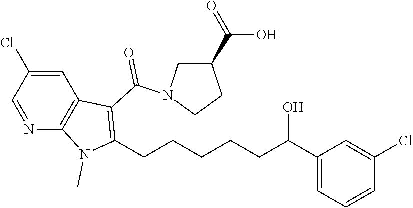 [SMILES: Cn1c(CCCCCC(O)c2cccc(Cl)c2)c(C(=O)N2CC[C@H](C(=O)O)C2)c2cc(Cl)cnc21] | 518.2 | ¹H NMR (400 MHz, DMSO-d₆) δ 8.24 (d, J = 2.4 Hz, 1H),, 7.91 (s, 1H), 7.34- 7.30 (m, 2H), 7.26-7.24 (m, 2H), 4.50 (t, J = 6.0 Hz, 1H), 3.76 (s, 3H), 3.58- 3.34 (m, 5H), 3.05 (s, 1H), 2.88 (t, J = 7.6 Hz, 2H), 2.07-2.01 (m, 2H), 1.56- 1.54 (m, 4H), 1.37-1.28 (m, 4H). |
| I-286 | 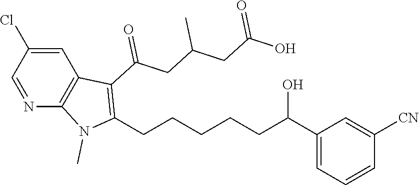 [SMILES: CC(CC(=O)O)CC(=O)c1c(CCCCCC(O)c2cccc(C#N)c2)n(C)c2ncc(Cl)cc12] | 496.3 | ¹H NMR (400 MHz, DMSO-d₆) δ 12.05 (br., 1H), 8.37 (s, 1H), 8.33 (s, 1H), 7.74 (s, 1H), 7.68-7.65 (m, 2H), 7.52 (t, J = 7.6 Hz, 1H), 5.37 (s, 1H), 4.59 (t, J = 6.4 Hz, 1H), 3.80 (s, 3H), 3.16 (t, J = 7.2 Hz, 2H), 3.03 (dd, J = 16.4, 5.2 Hz, 1H), 2.86 (dd, J = 16.4, 7.6 Hz, 1H), 2.48-2.44 (m, 1H), 2.39 (dd, J = 15.2, 5.6 Hz, 1H), 2.18 (dd, J = 15.2, 7.2 Hz, 1H), 1.63-1.58 (m, 4H), 1.46-1.37 (m, 3H), 1.35-1.28 (m, 1H), 0.96 (d, J = 6.4 Hz, 3H). |
| I-218 | 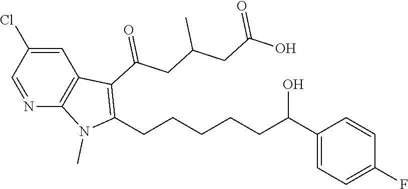 [SMILES: CC(CC(=O)O)CC(=O)c1c(CCCCCC(O)c2ccc(F)cc2)n(C)c2ncc(Cl)cc12] | 489.3 | ¹H NMR (400 MHz, DMSO-d₆) δ 12.09 (br., 1H), 8.37 (d, J = 2.0 Hz, 1H), 8.32 (d, J = 2.0 Hz, 1H), 7.35-7.31 (m, 2H), 7.10 (t, J = 8.8 Hz, 2H), 5.15 (s, 1H), 4.51 (t, J = 6.4 Hz, 1H), 3.79 (s, 3H), 3.16 (t, J = 7.2 Hz, 2H), 3.03 (dd, J = 16.4, 5.2 Hz, 1H), 2.86 (dd, J = 16.4, 7.6 Hz, 1H), 2.48-2.44 (m, 1H), 2.40 (dd, J = 15.2, 5.6 Hz, 1H), 2.18 (dd, J = 15.2, 8.0 Hz, 1H), 1.62-1.53 (m, 4H), 1.44- 1.36 (m, 3H), 1.29-1.22 (m, 1H), 0.96 (d, J = 6.4 Hz, 3H). |
| I-301 | 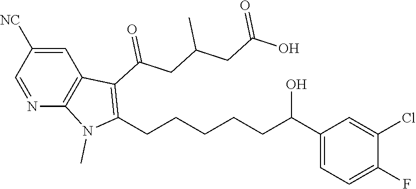 [SMILES: CC(CC(=O)O)CC(=O)c1c(CCCCCC(O)c2ccc(F)c(Cl)c2)n(C)c2ncc(C#N)cc12] | 514.2 | ¹H NMR (400 MHz, DMSO-d₆) δ 12.12 (br., 1H), 8.83 (d, J = 1.6 Hz, 1H), 8.73 (d, J = 1.6 Hz, 1H), 7.49-7.47 (m, 1H), 7.35-7.31 (m, 2H), 5.31 (br., 1H), 4.53 (t, J = 6.0 Hz, 1H), 3.84 (s, 3H), 3.18 (t, J = 7.2 Hz, 2H), 3.09 (dd, J = 16.8, 5.6 Hz, 1H), 2.91 (dd, J = 16.4, 7.6 Hz, 1H), 2.54-2.46 (m, 1H), 2.41 (dd, J = 15.2, 5.6 Hz, 1H), 2.18 (dd, J = 15.2, 7.6 Hz, 1H), 1.61-1.54 (m, 4H), 1.45-1.36 (m, 3H), 1.30-1.27 (m, 1H), 0.96 (d, J = 6.4 Hz, 3H). |
| I-302 | 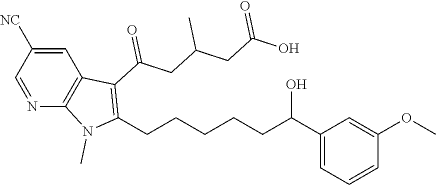 [SMILES: COc1cccc(C(O)CCCCCc2c(C(=O)CC(C)CC(=O)O)c3cc(C#N)cnc3n2C)c1] | 474.4 | ¹H NMR (400 MHz, DMSO-d₆) δ 12.10 (br, 1H), 8.82 (d, J = 1.6 Hz, 1H), 8.72 (d, J = 1.6 Hz, 1H), 7.19 (t, J = 8.0 Hz, 1H), 6.87-6.85 (m, 2H), 6.75 (d, J = 7.2 Hz, 1 H), 5.11 (br, 1H), 4.47 (t, J = 6.0 Hz, 1H), 3.84 (s, 3 H), 3.73 (s, 3 H), 3.18 (t, J = 8.0 Hz, 1H), 3.08 (dd, J = 16.8, 5.2 Hz, 1H), 2.91 (dd, J = 16.8, 7.6 Hz, 1H), 2.51-2.45 (m, 1H), 2.41 (dd, J = 15.6, 6.0 Hz, 1H), 2.18 (dd, J = 15.6, 8.0 Hz, 1H), 1.64-1.52 (m, 4H), 1.48-1.23 (m, 4H), 0.96 (d, J = 6.4 Hz, 3H). |
| I-110 | 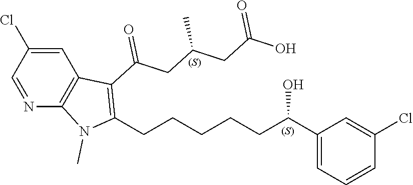 | 505.2 | ¹H NMR (400 MHz, DMSO-d₆) δ 12.09 (br., 1H), 8.37 (d, J = 2.0 Hz, 1H), 8.32 (d, J = 2.4 Hz, 1H), 7.36-7.34 (m, 1H), 7.31 (d, J = 7.6 Hz, 1H), 7.27-7.24 (m, 2H), 5.26 (s, 1H), 4.53 (t, J = 6.4 Hz, 1H), 3.80 (s, 3H), 3.16 (t, J = 7.6 Hz, 2H), 3.03 (dd, J = 16.8, 5.6 Hz, 1H), 2.86 (dd, J = 16.4, 7.6 Hz, 1H), 2.49- 2.45 (m, 1H), 2.40 (dd, J = 15.2, 5.6 Hz, 1H), 2.18 (dd, J = 15.2, 7.6 Hz, 1H), 1.61-1.54 (m, 4H), 1.44-1.37 (m, 3H), 1.35-1.28 (m, 1H), 0.96 (d, J = 6.4 Hz, 3H). |
| I-107 | 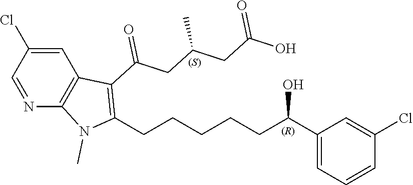 | 505.2 | ¹H NMR (400 MHz, DMSO-d₆) δ 12.10 (br., 1H), 8.37 (d, J = 2.0 Hz, 1H), 8.32 (d, J = 2.0 Hz, 1H), 7.36-7.34 (m, 1H), 7.31 (d, J = 7.6 Hz, 1H), 7.27-7.24 (m, 2H), 5.27 (s, 1H), 4.53 (t, J = 6.0 Hz, 1H), 3.80 (s, 3H), 3.16 (t, J = 7.2 Hz, 2H), 3.03 (dd, J = 16.4, 5.6 Hz, 1H), 2.86 (dd, J = 16.4, 7.6 Hz, 1H), 2.49- 2.45 (m, 1H), 2.40 (dd, J = 15.2, 5.6 Hz, 1H), 2.18 (dd, J = 15.6, 8.0 Hz, 1H), 1.61-1.57 (m, 4H), 1.44-1.37 (m, 3H), 1.34-1.28 (m, 1H), 0.97 (d, J = 6.4 Hz, 3H). |
| I-306 | 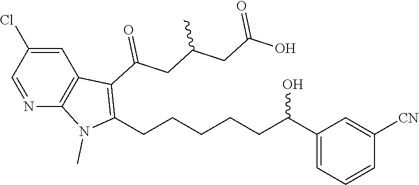 [SMILES: CC(CC(=O)O)CC(=O)c1c(CCCCCC(O)c2cccc(C#N)c2)n(C)c2ncc(Cl)cc12] | 496.3 | ¹H NMR (400 MHz, DMSO-d₆) δ 12.08 (br., 1H), 8.37 (d, J = 2.0 Hz, 1H), 8.33 (d, J = 2.4 Hz, 1H), 7.74 (s, 1H), 7.69- 7.65 (m, 2H), 7.52 (t, J = 7.6 Hz, 1H), 5.37 (br., 1H), 4.59 (t, J = 6.4 Hz, 1H), 3.80 (s, 3H), 3.16 (t, J = 7.6 Hz, 2H), 3.03 (dd, J = 16.8, 5.6 Hz, 1H), 2.86 (dd, J = 16.4, 7.6 Hz, 1H), 2.48-2.44 (m, 1H), 2.39 (dd, J = 15.2, 5.6 Hz, 1H), 2.17 (dd, J = 15.2, 7.2 Hz, 1H), 1.63- 1.58 (m, 4H), 1.46-1.37 (m, 3H), 1.35- 1.28 (m, 1H), 0.96 (d, J = 6.4 Hz, 3H). |
| I-307 | 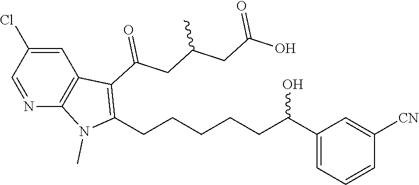 [SMILES: CC(CC(=O)O)CC(=O)c1c(CCCCCC(O)c2cccc(C#N)c2)n(C)c2ncc(Cl)cc12] | 496.3 | ¹H NMR (400 MHz, DMSO-d₆) δ 12.08 (br., 1H), 8.37 (d, J = 1.6 Hz, 1H), 8.33 (d, J = 2.0 Hz, 1H), 7.74 (s, 1H), 7.69- 7.65 (m, 2H), 7.52 (t, J = 7.6 Hz, 1H), 5.37 (br., 1H), 4.59 (t, J = 6.4 Hz, 1H), 3.80 (s, 3H), 3.16 (t, J = 7.6 Hz, 2H), 3.03 (dd, J = 16.8, 5.6 Hz, 1H), 2.86 (dd, J = 16.4, 7.6 Hz, 1H), 2.48-2.44 (m, 1H), 2.39 (dd, J = 15.2, 5.6 Hz, 1H), 2.17 (dd, J = 15.6, 8.0 Hz, 1H), 1.63- 1.58 (m, 4H), 1.43-1.37 (m, 3H), 1.34- 1.28 (m, 1H), 0.96 (d, J = 6.4 Hz, 3H). |
| I-308 | 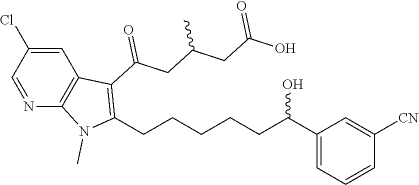 [SMILES: CC(CC(=O)O)CC(=O)c1c(CCCCCC(O)c2cccc(C#N)c2)n(C)c2ncc(Cl)cc12] | 496.3 | ¹H NMR (400 MHz, DMSO-d₆) δ 12.10 (br., 1H), 8.37 (d, J = 2.0 Hz, 1H), 8.33 (d, J = 2.0 Hz, 1H), 7.74 (s, 1H), 7.69- 7.65 (m, 2H), 7.52 (t, J = 7.6 Hz, 1H), 5.37 (s, 1H), 4.59 (t, J = 6.4 Hz, 1H), 3.80 (s, 3H), 3.16 (t, J = 7.2 Hz, 2H), 3.03 (dd, J = 16.4, 5.2 Hz, 1H), 2.86 (dd, J = 16.4, 7.6 Hz, 1H), 2.47-2.44 (m, 1H), 2.39 (dd, J = 15.2, 5.6 Hz, 1H), 2.17 (dd, J = 15.6, 8.0 Hz, 1H), 1.63- 1.54 (m, 4H), 1.45-1.37 (m, 3H), 1.34- 1.30 (m, 1H), 0.96 (d, J = 6.4 Hz, 3H). |
| I-309 | 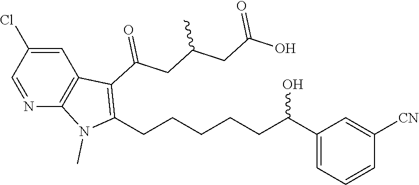 [SMILES: CC(CC(=O)O)CC(=O)c1c(CCCCCC(O)c2cccc(C#N)c2)n(C)c2ncc(Cl)cc12] | 496.3 | ¹H NMR (400 MHz, DMSO-d₆) δ 12.11 (br., 1H), 8.36 (d, J = 2.0 Hz, 1H), 8.33 (d, J = 2.0 Hz, 1H), 7.74 (s, 1H), 7.69- 7.65 (m, 2H), 7.52 (t, J = 7.6 Hz, 1H), 5.40 (s, 1H), 4.60 (t, J = 6.4 Hz, 1H), 3.80 (s, 3H), 3.16 (t, J = 6.8 Hz, 2H), 3.03 (dd, J = 16.4, 5.2 Hz, 1H), 2.86 (dd, J = 16.8, 7.6 Hz, 1H), 2.48-2.45 (m, 1H), 2.39 (dd, J = 15.6, 5.6 Hz, 1H), 2.18 (dd, J = 15.6, 8.0 Hz, 1H), 1.63- 1.53 (m, 4H), 1.46-1.37 (m, 3H), 1.34- 1.28 (m, 1H), 0.97 (d, J = 6.4 Hz, 3H). |
| I-310 | 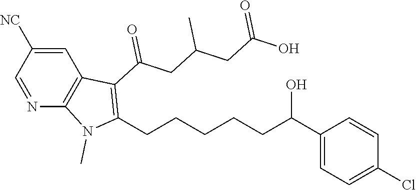 [SMILES: CC(CC(=O)O)CC(=O)c1c(CCCCCC(O)c2ccc(Cl)cc2)n(C)c2ncc(C#N)cc12] | 496.2 | ¹H NMR (400 MHz, DMSO-d₆) δ 12.09 (br., 1H), 8.83 (d, J = 1.6 Hz, 1H), 8.73 (d, J = 1.6 Hz, 1H), 7.41-7.31 (m, 4H), 5.21 (s, 1H), 4.52 (t, J = 6.8 Hz, 1H), 3.84 (s, 3H), 3.49-3.41 (m, 1H), 3.22-3.14 (m, 2H), 3.09 (dd, J = 16.4 Hz, 6.4 Hz, 1H), 2.90 (dd, J = 16.4 Hz, 8.0 Hz, 1H), 2.41 (dd, J = 15.6 Hz, 5.6 Hz, 1H), 2.20 (dd, J = 15.6 Hz, 8.0 Hz, 1H), 1.63-1.51 (m, 4H), 1.47-1.35 (m, 3H), 1.34-1.21 (m, 1H), 0.96 (d, J = 6.4 Hz, 3H). |
| I-311 | 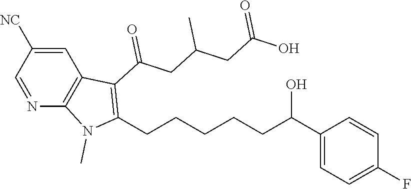 [SMILES: CC(CC(=O)O)CC(=O)c1c(CCCCCC(O)c2ccc(F)cc2)n(C)c2ncc(C#N)cc12] | 480.3 | ¹H NMR (400 MHz, DMSO-d₆) δ 8.84 (s, 1H), 8.72 (s, 1H), 7.33 (dd, J = 8.8 Hz, 6.0 Hz, 2H), 7.10 (d, J = 8.8 Hz, 2H), 4.51 (t, J = 6.0 Hz, 1H), 3.84 (s, 3H), 3.18 (t, J = 8.0 Hz, 2H), 3.10 (dd, J = 16.4, 4.8 Hz, 1H), 2.88 (dd, J = 16.4, 6.8 Hz, 1H), 2.48-2.44 (m, 1H), 2.39- 2.35 (m, 1H), 2.15 (dd, J = 15.2, 7.6 Hz, 1H), 1.61-1.53 (m, 4H), 1.45-1.37 (m, 3H), 1.31-1.25 (m, 1H), 0.95 (d, J = 6.8 Hz, 3H). |
| I-312 | 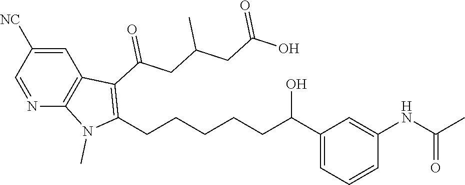 [SMILES: CC(=O)Nc1cccc(C(O)CCCCCc2c(C(=O)CC(C)CC(=O)O)c3cc(C#N)cnc3n2C)c1] | 519.3 | ¹H NMR (400 MHz, DMSO-d₆) δ 12.07 (br, 1H), 9.90 (s, 1H), 8.83 (d, J = 1.6 Hz, 1H), 8.73 (d, J = 1.6 Hz, 1H), 7.50 (s, 1H), 7.46 (d, J = 8.4 Hz, 1H), 7.19 (t, J = 7.6 Hz, 1H), 6.95 (d, J = 7.6 Hz, 1H), 5.13 (br., 1H), 4.46 (t, J = 6.4 Hz, 1H), 3.84 (s, 3H), 3.19 (t, J = 7.6 Hz, 2H), 3.09 (dd, J = 16.8 Hz, J = 5.6 Hz, 1H), 2.90 (dd, J = 16.8 Hz, J = 8.0 Hz, 1H), 2.51-2.45 (m, 1H), 2.40 (dd, J = 15.2 Hz, J = 5.6 Hz, 1H), 2.17 (dd, J = 15.2 Hz, J = 8.0 Hz, 1H), 2.02 (s, 3H), |
| | | | 1.61-1.52 (m, 4H), 1.44-1.30 (m, 4H), |
| | | | 0.96 (d, J = 6.4 Hz, 3H). |
| I-313 | 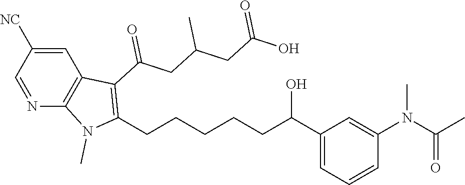 [SMILES: CC(=O)N(C)c1cccc(C(O)CCCCCc2c(C(=O)CC(C)CC(=O)O)c3cc(C#N)cnc3n2C)c1] | 533.3 | ¹H NMR (400 MHz, DMSO-d₆) δ 12.15 (br., 1H), 8.83 (s, 1H), 8.74 (d, J = 0.8 Hz, 1H), 7.39-7.35 (m, 1H), 7.29 (d, J = 7.2 Hz, 1H), 7.23 (s, 1H), 7.16 (d, J = 7.2 Hz, 1H), 5.24 (br, 1H), 4.54 (t, J = 6.4 Hz, 1H), 3.84 (s, 3H), 3.20-3.06 (m, 6H), 2.90 (dd, J = 17.2 Hz, 8.4 Hz, 1H), 2.49-2.42 (m, 2H), 2.17 (dd, J = 15.6 Hz, 8.0 Hz, 1H), 1.74 (s, 3H), 1.60-1.58 (m, 4H), 1.42-1.30 (m, 4H), 0.95 (d, J = 6.8 Hz, 3H). |
| I-314 | 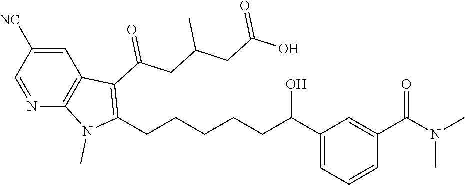 [SMILES: CC(CC(=O)O)CC(=O)c1c(CCCCCC(O)c2cccc(C(=O)N(C)C)c2)n(C)c2ncc(C#N)cc12] | 533.3 | ¹H NMR (400 MHz, DMSO-d₆) δ 12.05 (br., 1H), 8.82 (d, J = 1.6 Hz, 1H), 8.72 (d, J = 7.6 Hz, 1H), 7.37-7.33 (m, 3H), 7.24-7.22 (m, 1H), 5.25 (br, 1H), 4.56 (t, J = 6.0 Hz, 1H), 3.84 (s, 2H), 3.18 (t, J = 8.0 Hz, 1H), 3.09 (dd, J = 16.8 Hz, J = 5.2 Hz, 1H), 2.98-2.90 (m, 7H), 2.51-2.46 (m, 1H), 2.42 (dd, J = 15.6 Hz, 6.0 Hz, 1H), 2.19 (dd, J = 15.6 Hz, 8.0 Hz, 1H), 1.61-1.59 (m, 4H), 1.43- 1.27 (m, 4H), 0.97 (d, J = 6.4 Hz, 3H). |
| I-315 | 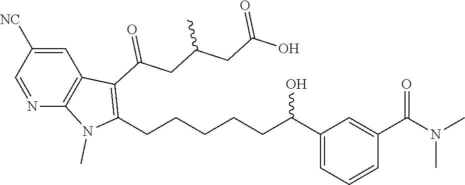 [SMILES: CC(CC(=O)O)CC(=O)c1c(CCCCCC(O)c2cccc(C(=O)N(C)C)c2)n(C)c2ncc(C#N)cc12] | 533.2 | ¹H NMR (400 MHz, DMSO-d₆) δ 8.83 (d, J = 1.6 Hz, 1H), 8.72 (d, J = 2.0 Hz, 1H), 7.36-7.32 (m, 3H), 7.23-7.21 (m, 1H), 4.55 (t, J = 5.6 Hz, 1H), 3.84 (s, 3H), 3.20 (t, J = 8.0 Hz, 2H), 3.10 (dd, J = 16.8 Hz, 5.6 Hz, 1H), 2.97-2.84 (m, 7H), 2.36 (dd, J = 15.6 Hz, 6.4 Hz, 1H), 2.15 (dd, J = 15.2 Hz, 8.0 Hz, 1H), 1.61- 1.57 (m, 4H), 1.42-1.24 (m, 6H), 0.95 (d, J = 6.8 Hz, 3H). |
| I-316 | 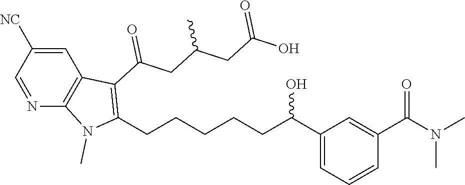 [SMILES: CC(CC(=O)O)CC(=O)c1c(CCCCCC(O)c2cccc(C(=O)N(C)C)c2)n(C)c2ncc(C#N)cc12] | 533.2 | ¹H NMR (400 MHz, DMSO-d₆) δ 8.83 (d, J = 1.6 Hz, 1H), 8.72 (d, J = 2.0 Hz, 1H), 7.36-7.32 (m, 3H), 7.23-7.21 (m, 1H), 4.55 (t, J = 6.0 Hz, 1H), 3.84 (s, 3H), 3.18 (t, J = 8.0 Hz, 2H), 3.10 (dd, J = 16.8 Hz, 5.6 Hz, 1H), 2.97-2.85 (m, 7H), 2.38 (dd, J = 15.6 Hz, 6.0 Hz, 1H), 2.17 (dd, J = 15.6 Hz, 8.0 Hz, 1H), 1.61- 1.58 (m, 4H), 1.42-1.23 (m, 6H), 0.95 (d, J = 6.4 Hz, 3H). |
| I-317 | 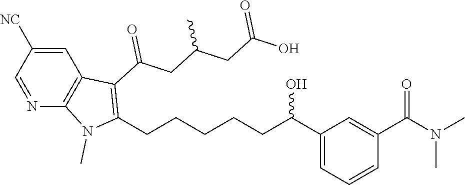 [SMILES: CC(CC(=O)O)CC(=O)c1c(CCCCCC(O)c2cccc(C(=O)N(C)C)c2)n(C)c2ncc(C#N)cc12] | 533.3 | ¹H NMR (400 MHz, DMSO-d₆) δ 9.02 (d, J = 1.6 Hz, 1H), 8.71 (d, J = 1.6 Hz, 1H), 7.38-7.32 (m, 3H), 7.21 (d, J = 6.8 Hz, 1H), 5.91 (br., 1H), 4.57 (t, J = 5.6 Hz, 1H), 3.84 (s, 3H), 3.30-3.11 (m, 3H), 2.97 (s, 3H), 2.89 (s, 3H), 2.60- 2.50 (m, 1H), 2.39-2.33 (m, 1H), 1.90 (d, J = 6.8 Hz, 2H), 1.60-1.56 (m, 4H), 1.45-1.23 (m, 4H), 0.86 (d, J = 6.4 Hz, 3H). |
| I-318 | 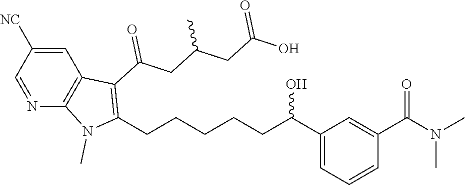 [SMILES: CC(CC(=O)O)CC(=O)c1c(CCCCCC(O)c2cccc(C(=O)N(C)C)c2)n(C)c2ncc(C#N)cc12] | 533.3 | ¹H NMR (400 MHz, DMSO-d₆) δ 8.83 (s, 1H), 8.73 (s, 1H), 7.36-7.32 (m, 3H), 7.22 (d, J = 6.0 Hz, 1H), 5.91 (br., 1H), 4.55 (t, J = 6.0 Hz, 1H), 3.84 (s, 3H), 3.18 (t, J = 8.0 Hz, 2H), 3.11 (dd, J = 17.2 Hz, 5.2 Hz, 1H), 2.97 (s, 3H), 2.91- 2.85 (m, 4H), 2.37-2.33 (m, 2H), 2.13 (dd, J = 15.6 Hz, 6.8 Hz, 1H), 1.60-1.58 (m, 4H), 1.42-1.23 (m, 4H), 0.95 (d, J = 6.4 Hz, 3H). |

TABLE 4a
Additional Characterization Data for Additional Exemplary Compound

| Compound | Rt (Min) | Rt (Min) |
|---|---|---|
| No. | (LCMS) | (HPLC) |
| I-306 | 1.852 | 9.096 |
| I-307 | 1.851 | 9.091 |
| I-308 | 1.852 | 9.095 |
| I-309 | 1.851 | 9.094 |
| I-310 | 1.868 | 9.179 |
| I-311 | 1.788 | 8.689 |
| I-312 | 1.588 | 7.422 |
| I-313 | 1.615 | 7.577 |
| I-314 | 1.612 | 7.536 |
| I-315 | 1.612 | 7.586 |
| I-316 | 1.612 | 7.583 |
| I-317 | 1.611 | 7.556 |
| I-318 | 1.608 | 7.562 |

### Example 4: Synthesis of Compound I-68

### Synthetic Scheme for Compound 1-68

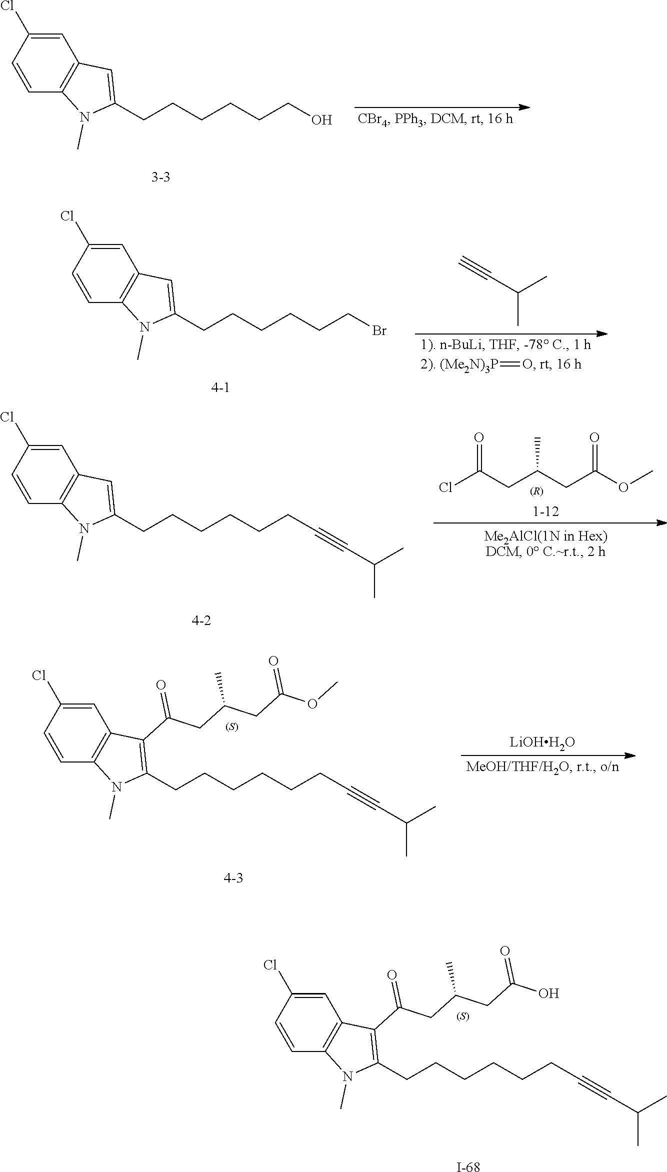

### 4.1 The Synthesis of Intermediate 4-1

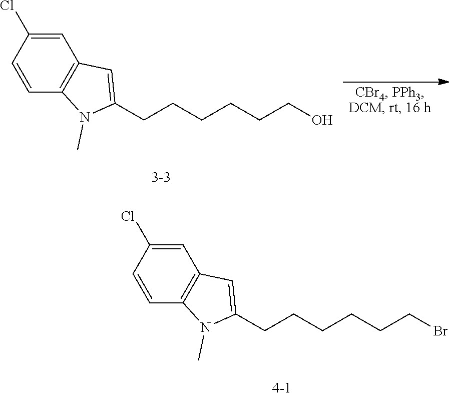 [SMILES: Cn1c(CCCCCCO)cc2cc(Cl)ccc21 ; "CBr4, PPh3,DCM, rt, 16 h" ; Cn1c(CCCCCCBr)cc2cc(Cl)ccc21]

To a stirred solution of 3-3 (505 mg, 1.9 mmol) in DCM (10 mL) was added CBr₄ (945 mg, 2.9 mmol) and PPh₃ (746 mg, 2.9 mmol) at 0° C. The reaction mixture was stirred at room temperature for 16 hours until the reaction was complete. The solvent was evaporated under reduced pressure and the crude was purified by silica gel chromatography (PE/EA=1/0 to 1/1) to afford 4-1 (410 mg, yield: 66%) as colorless oil. LC-MS m/z: 328.0 [M+H]⁺.

### 4.2 The Synthesis of Intermediate 4-2

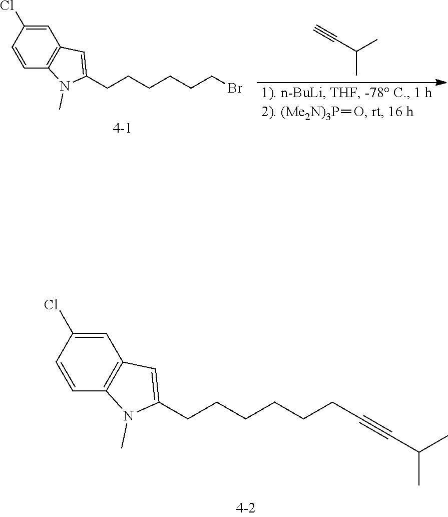

To a suspension of 3-methylbut-1-yne (73 mg, 1.1 mmol) in THF (3 mL) was added n-BuLi (2.5 M in THF, 0.5 mL, 1.3 mmol) at −78° C. The mixture was stirred for 1 hour and 4-1 (350 mg, 1.1 mmol) in HMPA (1 mL) was added dropwise. The reaction mixture was allowed to warm to rt and stirred for another 16 h. Saturated NH₄Cl solution was added and the reaction mixture was extracted with EtOAc. The combined organic layer was washed with brine, dried over anhydrous Na₂SO₄ and the solvent was evaporated under reduced pressure. The crude was purified by silica gel chromatography (0% to 33% EtOAc/Hexane) to afford 4-2 (280 mg, yield: 83%) as yellow oil. ¹H NMR (400 MHz, CDCl₃) δ 7.47 (s, 1H), 7.15 (d, J=8.8 Hz, 1H), 7.08 (d, J=8.8 Hz, 1H), 6.18 (s, 1H), 3.64 (s, 3H), 2.71 (t, J=7.6 Hz, 2H), 2.52-2.50 (m, 1H), 2.16-2.14 (m, 2H), 1.72-1.70 (m, 2H), 1.49-1.47 (m, 6H), 1.13 (d, J=6.8 Hz, 6H).

### 4.3 The Synthesis of Intermediate 4-3

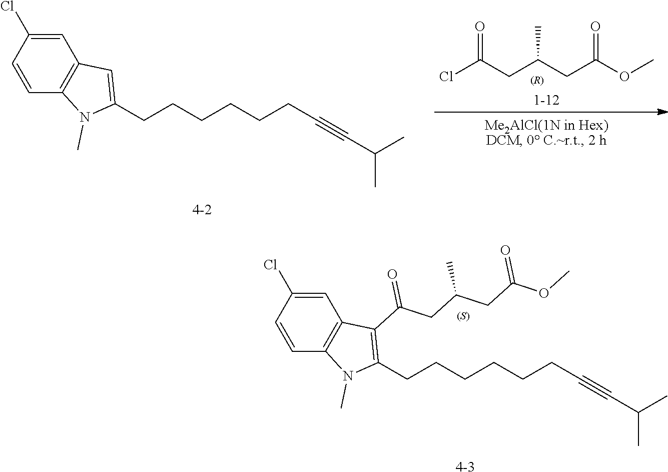

To a stirred solution of 4-2 (200 mg, 0.44 mmol) in dichloromethane (3 mL) was added Me₂AlCl (1.0 M in hexane, 0.9 mL, 0.87 mmol) at 0° C., the mixture was stirred for another 30 min. Then 1-12 (117 mg, 0.66 mmol) in dichloromethane (1 mL) was added dropwise at 0° C. and the reaction mixture was allowed to warm to room temperature and stirred for another 2 h. The reaction was quenched by adding water and extracted with EtOAc. The organic layer was combined, washed with brine and dried over anhydrous Na₂SO₄. The solvent was evaporated under reduced pressure to afford 4-3 (280 mg, crude) as colorless oil, the crude was used to next step directly.

### 4.4 The Synthesis of Compound I-68

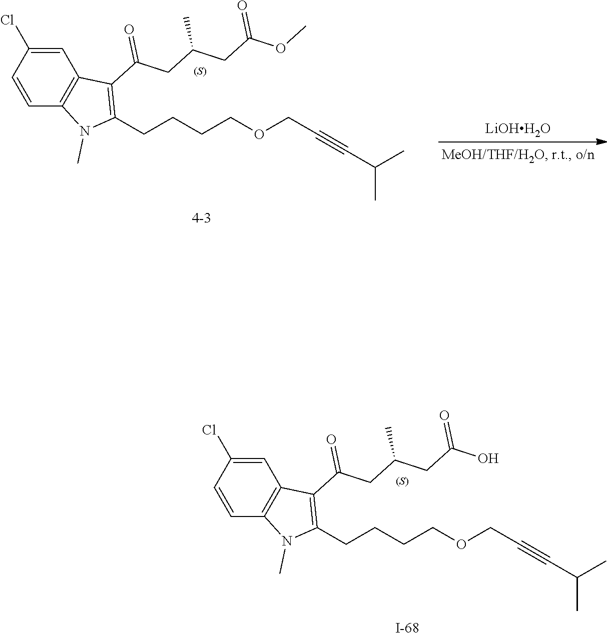

To a stirred solution of 4-3 (280 mg, 0.61 mmol) in MeOH/THF/H₂O (2 mL/2 mL/0.2 mL) was added Lithium hydroxide monohydrate (257 mg, 6.1 mmol). The reaction mixture was stirred at rt overnight until the reaction was complete (by LCMS). Saturated NH₄Cl solution was added at 0° C. and the mixture was acidified with HCl (1.0 M) to pH ˜3.0 then extracted with EtOAc, the combined organic layer was washed with brine, dried over anhydrous Na₂SO₄ and evaporated under reduced pressure. The crude was purified by Prep-HPLC to afford I-68 (112 mg, yield: 41%) as white semi-solid. ¹H NMR (400 MHz, CDCl₃) δ 7.88 (s, 1H), 7.23-7.22 (m, 1H), 7.20 (dd, J=8.8, 1.6 Hz, 1H), 3.70 (s, 3H), 3.18-3.14 (m, 2H), 3.00-2.96 (m, 2H), 2.73-2.71 (m, 1H), 2.53-2.51 (m, 2H), 2.35 (dd, J=15.6, 7.2 Hz, 1H), 2.14-2.13 (m, 2H), 1.60-1.58 (m, 2H), 1.48-1.46 (m, 6H), 1.14-1.11 (m, 9H). LC-MS m/z: 444.0 [M+H]⁺.

Additional exemplary compounds were prepared following methods substantially similar to the method described above and herein. Data for these compounds are provided in Tables 5 and 5a below.

TABLE 5
Characterization Data for Additional Exemplary Compounds

| Compound | | | |
|---|---|---|---|
| No. | Chemical Structure | M + 1 | ¹H NMR (400 MHz) |
| I-111 | 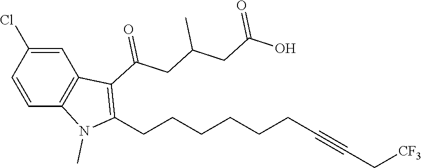 [SMILES: CC(CC(=O)O)CC(=O)c1c(CCCCCCC#CCC(F)(F)F)n(C)c2ccc(Cl)cc12] | 484.0 | ¹H NMR (400 MHz, CDCl₃) δ 7.81 (d, J = 1.6 Hz, 1H), 7.18-7.12 (m, 2H), 3.65 (s, 3H), 3.16-3.06 (m, 2H), 3.01-2.86 (m, 4H), 2.73- 2.63 (m, 1H), 2.47 (dd, J = 14.8, 5.6 Hz, 1H), 2.29 (dd, J = 15.2, 7.2 Hz, 1H), 2.17-2.07 (m, 2H), 1.65- 1.53 (m, 2H), 1.49-1.37 (m, 6H), 1.08 (d, J = 6.4 Hz, 3H). |
| I-304 | 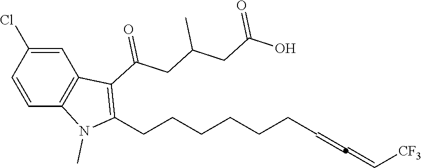 | 483.9 | ¹H NMR (400 MHz, DMSO-d₆) δ 12.07 (s, 1H), 7.96 (d, J = 1.6 Hz, 1H), 7.59 (d, J = 8.8 Hz, 1H), 7.25 (dd, J = 8.8, 1.6 Hz, 1H), 5.59-5.48 (m, 2H), 3.76 (s, 3H), 3.16-3.12 (m, 2H), 2.98 (dd, J = 16.4, 5.6 Hz, 1H), 2.84 (dd, J = 16.4, 7.6 Hz, 1H), 2.51-2.47 (m, 1H), 2.39 (dd, J = 15.2, 5.6 Hz, 1H), 2.24-2.09 (m, 3H), 1.62-1.53 (m, 2H), 1.50- 1.37 (m, 6H), 0.97 (d, J = 6.4 Hz, 3H). |
| I-112 | 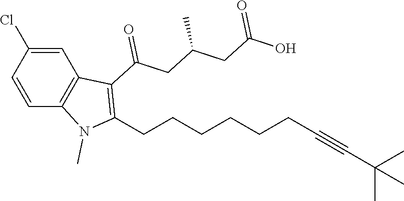 [SMILES: C[C@H](CC(=O)O)CC(=O)c1c(CCCCCCC#CC(C)(C)C)n(C)c2ccc(Cl)cc12] | 458.0 | ¹H NMR (400 MHz, DMSO-d₆) δ 12.10 (s, 1H), 7.96 (d, J = 1.6 Hz, 1H), 7.59 (d, J = 8.8 Hz, 1H), 7.25 (dd, J = 8.8 Hz, 2.0 Hz, 1H), 3.76 (s, 3H), 3.13 (t, J = 8.0 Hz, 2H), 2.99 (dd, J = 16.4, 6.0 Hz, 1H), 2.84 (dd, J = 16.4, 7.6 Hz, 1H), 2.55-2.52 (m, 1H), 2.39 (dd, J = 15.6, 6.0 Hz, 1H), 2.23-2.05 (m, 3H), 1.59-1.52 (m, 2H), 1.45-1.34 (m, 6H), 1.14 (s, 9H), 0.97 (d, J = 6.4 Hz, 3H). |
| I-114 | 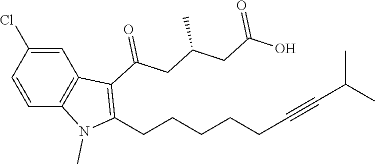 [SMILES: CC(C)C#CCCCCCc1c(C(=O)C[C@H](C)CC(=O)O)c2cc(Cl)ccc2n1C] | 430.0 | ¹H NMR (400 MHz, DMSO-d₆) δ 12.07 (s, 1H), 7.96 (d, J = 2.0 Hz, 1H), 7.59 (d, J = 8.8 Hz, 1H), 7.25 (dd, J = 8.8, 2.0 Hz, 1H), 3.76 (s, 3H), 3.32 (s, 1H), 3.15 (t, J = 7.6 Hz, 2H), 2.99 (dd, J = 16.4, 5.6 Hz, 1H), 2.85 (dd, J = 16.4, 7.6 Hz, 1H), 2.48-2.46 (m, 1H), 2.39 (dd, J = 15.2, 5.6 Hz, 1H), 2.20- 2.13 (m, 3H), 1.58 (br, 2H), 1.48- 1.43 (m, 4H), 1.05 (d, J = 6.4 Hz, 6H), 0.98 (d, J = 6.4 Hz, 3H). |
| I-278 | 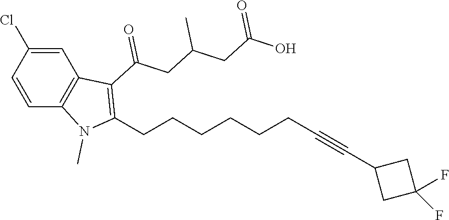 [SMILES: CC(CC(=O)O)CC(=O)c1c(CCCCCCC#CC2CC(F)(F)C2)n(C)c2ccc(Cl)cc12] | 492.4 | ¹H NMR (400 MHz, DMSO-d₆) δ 12.08 (br., 1H), 7.95 (d, J = 1.6 Hz, 1H), 7.59 (d, J = 8.8 Hz, 1H), 7.25 (dd, J = 8.8, 2.0 Hz, 1H), 3.76 (s, 3H), 3.14 (t, J = 7.2 Hz, 2H), 3.01-2.82 (m, 5H), 2.59-2.54 (m, 2H), 2.50-2.45 (m, 1H), 2.38 (dd, J = 15.6, 6.0 Hz, 1H), 2.20-2.08 (m, 3H), 1.60-1.52 (m, 2H), 1.50- 1.39 (m, 6H), 0.97 (d, J = 6.8 Hz, 3H). |
| I-116 | 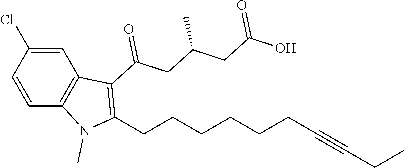 [SMILES: CCC#CCCCCCCc1c(C(=O)C[C@H](C)CC(=O)O)c2cc(Cl)ccc2n1C] | 430.0 | ¹H NMR (400 MHz, DMSO-d₆) δ 12.12 (s, 1H), 7.96 (s, 1H), 7.59 (d, J = 8.8 Hz, 1H), 7.25 (dd, J = 7.2, 1.2 Hz, 1H), 3.76 (s, 3H), 3.13 (t, J = 7.6 Hz, 2H), 2.99 (dd, J = 16.4, 6.0 Hz, 1H), 2.84 (dd, J = 16.4, 7.6 Hz, 1H), 2.51-2.49 (m, 1H) , 2.38 (dd, J = 15.2, 5.6 Hz, 1H), 2.22- 2.06 (m, 5H), 1.56 (s, 2H), 1.41 (s, 6H), 1.02 (t, J = 7.2 Hz, 3H), 0.97 (t, J = 6.8 Hz, 3H). |
| I-117 | 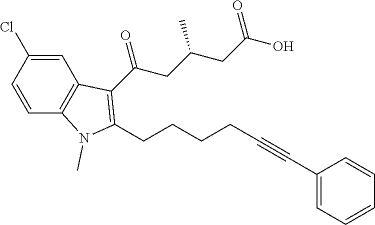 [SMILES: C[C@H](CC(=O)O)CC(=O)c1c(CCCCC#Cc2ccccc2)n(C)c2ccc(Cl)cc12] | 450.0 | ¹H NMR (400 MHz, MeOD) δ7.94 (d, J = 2.0 Hz, 1H), 7.42 (d, J = 8.8 Hz, 1H), 7.34-7.30 (m, 2H), 7.28-7.25 (m, 3H), 7.21 (dd, J = 8.4, 2.0 Hz, 1H), 3.78 (s, 3H), 3.27 (t, J = 7.2 Hz, 2H), 3.06 (dd, J = 16.0, 6.4 Hz, 1H), 2.89 (dd, J = 16.0, 7.6 Hz, 1H), 2.68-2.60 (m, 1H), 2.53-2.43 (m, 3H), 2.26 (dd, J = 15.2, 7.6 Hz, 1H), 1.86-1.75 (m, 4H), 1.06 (d, J = 6.4 Hz, 3H). |
| I-176 | 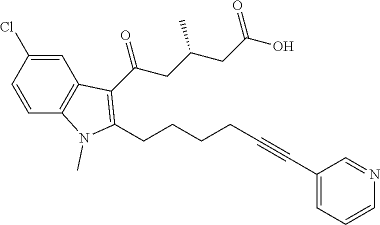 [SMILES: C[C@H](CC(=O)O)CC(=O)c1c(CCCCC#Cc2cccnc2)n(C)c2ccc(Cl)cc12] | 451.2 | ¹H NMR (400 MHz, DMSO-d₆) δ 12.10 (s, 1H), 8.57 (d, J = 1.2 Hz, 1H), 8.51 (dd, J = 4.8, 1.6 Hz, 1H), 7.96 (d, J = 2.0 Hz, 1H), 7.79 (dt, J = 8.0, 2.0 Hz, 1H), 7.60 (d, J = 8.8 Hz, 1H), 7.41-7.33 (m, 1H), 7.25 (dd, J = 8.8, 2.0 Hz, 1H), 3.79 (s, 3H), 3.22 (t, J = 7.2 Hz, 2H), 3.01 (dd, J = 16.4, 5.6 Hz, 1H), I- 1392.86 (dd, J = 16.4, 7.6 Hz, 1H), 2.55 (t, J = 6.4 Hz, 2H), 2.49-2.43 (m, 1H), 2.38 (dd, J = 15.2, 5.6 Hz, 1H), 2.16 (dd, J = 15.2, 8.0 Hz, 1H), 1.82-1.63 (m, 4H), 0.96 (d, J = 6.4 Hz, 3H). |
| I-227 | 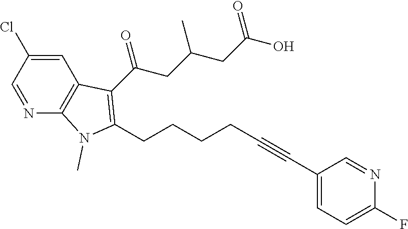 [SMILES: CC(CC(=O)O)CC(=O)c1c(CCCCC#Cc2ccc(F)nc2)n(C)c2ncc(Cl)cc12] | 470.1 | ¹H NMR (400 MHz, DMSO-d₆) δ 12.10 (br., 1H), 8.38 (d, J = 2.0 Hz, 1H), 8.33 (d, J = 2.0 Hz, 1H), 8.27 (d, J = 1.6 Hz, 1H), 7.99 (dt, J = 8.0, 2.4 Hz, 1H), 7.19 (dd, J = 8.4, 2.8 Hz, 1H), 3.84 (s, 3H), 3.27 (t, J = 7.2 Hz, 2H), 3.05 (dd, J = 16.8, 5.6 Hz, 1H), 2.88 (dd, J = 16.4, 7.6 Hz, 1H), 2.54 (t, J = 6.4 Hz, 2H), 2.49-2.47 (m, 1H), 2.39 (dd, J = 15.6, 6.0 Hz, 1H), 2.17 (dd, J = 15.6, 8.0 Hz, 1H), 1.80- 1.69 (m, 4H), 0.95 (d, J = 6.8 Hz, 3H). |
| I-228 | 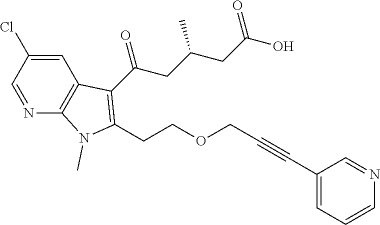 [SMILES: C[C@H](CC(=O)O)CC(=O)c1c(CCOCC#Cc2cccnc2)n(C)c2ncc(Cl)cc12] | 454.3 | ¹H NMR (400 MHz, DMSO-d₆) δ 8.56-8.55 (m, 2H), 8.33 (dd, J = 11.6, 2.0 Hz, 2H), 7.77 (dt, J = 8.0, 1.6 Hz, 1H), 7.39 (dd, J = 7.2, 4.8 Hz, 1H), 4.41 (s, 2H), 3.90- 3.87 (m, 5H), 3.51 (t, J = 6.0 Hz, 2H), 3.06 (dd, J = 16.8, 5.6 Hz, 1H), 2.86 (dd, J = 16.4, 7.6 Hz, 1H), 2.48-2.45 (m, 1H), 2.38 (dd, J = 15.2, 5.6 Hz, 1H), 2.16 (dd, J = 15.2, 7.6 Hz, 1H), 0.95 (d, J = 6.4 Hz, 3H). |
| I-229 | 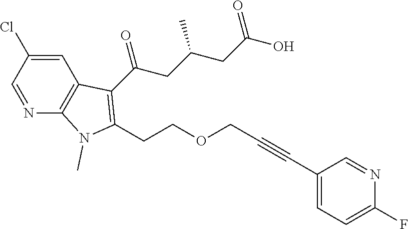 [SMILES: C[C@H](CC(=O)O)CC(=O)c1c(CCOCC#Cc2ccc(F)nc2)n(C)c2ncc(Cl)cc12] | 472.1 | ¹H NMR (400 MHz, DMSO-d₆) δ 12.11 (br., 1H), 8.33 (dd, J = 9.6, 2.0 Hz, 2H), 8.26 (d, J = 1.6 Hz, 1H), 7.97 (dt, J = 8.0, 2.4 Hz, 1H), 7.22 (dd, J = 8.4, 2.8 Hz, 1H), 4.40 (s, 2H), 3.90-3.87 (m, 5H), 3.51 (t, J = 6.0 Hz, 2H), 3.06 (dd, J = 16.4, 5.2 Hz, 1H), 2.88 (dd, J = 16.8, 7.6 Hz, 1H), 2.51-2.47 (m, 1H), 2.40 (dd, J = 15.2, 5.6 Hz, 1H), 2.17 (dd, J = 15.2, 7.6 Hz, 1H), 0.96 (d, J = 6.4 Hz, 3H). |
| I-319 | 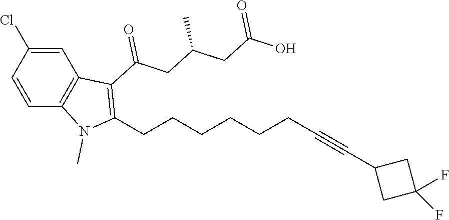 [SMILES: C[C@H](CC(=O)O)CC(=O)c1c(CCCCCCC#CC2CC(F)(F)C2)n(C)c2ccc(Cl)cc12] | 492.3 | 1H NMR (400 MHz, DMSO-d6) δ 12.09 (s, 1H), 7.96 (d, J = 2.4 Hz, 1H), 7.59 (d, J = 8.8 Hz, 1H), 7.25 (dd, J = 8.8, 2.0 Hz, 1H), 3.76 (s, 3H), 3.14 (t, J = 7.6 Hz, 2H), 3.00- 2.82 (m, 5H), 2.56-2.54 (m, 2H), 2.49-2.46 (m, 1H), 2.38 (dd, J = 15.6, 5.6 Hz, 1H), 2.20-2.14 (m, 3H), 1.60-1.52 (m, 2H), 1.50-1.38 (m, 6H), 0.97 (d, J = 6.8 Hz, 3H). |
| I-320 | 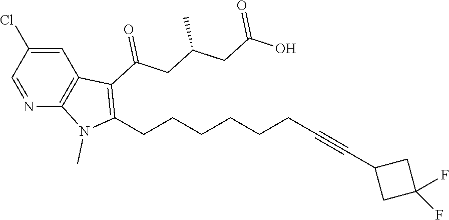 [SMILES: C[C@H](CC(=O)O)CC(=O)c1c(CCCCCCC#CC2CC(F)(F)C2)n(C)c2ncc(Cl)cc12] | 493.1 | 1H NMR (400 MHz, DMSO-d6) δ 12.09 (s, 1H), 8.37 (d, J = 2.0 Hz, 1H), 8.33 (d, J = 2.4 Hz, 1H), 3.81 (s, 3H), 3.19 (t, J = 7.6 Hz, 2H), 3.04 (dd, J = 16.4, 5.6 Hz, 1H), 2.94-2.84 (m, 5H), 2.56-2.50 (m, 2H), 2.49-2.45 (m, 1H), 2.39 (dd, J = 15.2, 5.6 Hz, 1H), 2.21-2.15 (m, 3H), 1.65-1.56 (m, 2H), 1.47- 1.38 (m, 6H), 0.97 (d, J = 6.8 Hz, 3H). |
| I-321 | 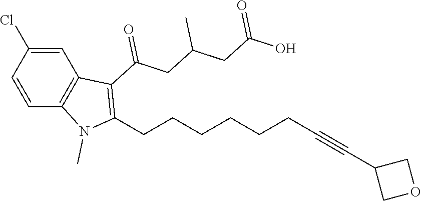 [SMILES: CC(CC(=O)O)CC(=O)c1c(CCCCCCC#CC2COC2)n(C)c2ccc(Cl)cc12] | 458.3 | 1H NMR (400 MHz, DMSO-d6) δ 12.09 (s, 1H), 7.95 (d, J = 1.6 Hz, 1H), 7.59 (d, J = 8.8 Hz, 1H), 7.25 (dd, J = 8.4, 2.0 Hz, 1H), 4.68 (dd, J = 8.4, 5.6 Hz, 2H), 4.45 (dd, J = 7.2, 5.6 Hz, 2H), 3.86-3.81 (m, 1H), 3.76 (s, 3H), 3.14 (t, J = 8.0 Hz, 2H), 2.98 (dd, J = 16.0, 5.6 Hz, 1H), 2.85 (dd, J = 16.4, 7.6 Hz, 1H), 2.49-2.46 (m, 1H), 2.38 (dd, J = 15.2, 5.6 Hz, 1H), 2.22-2.14 (m, 3H), 1.60-1.52 (m, 2H), 1.50- 1.38 (m, 6H), 0.97 (d, J = 6.4 Hz, 3H). |
| I-322 | 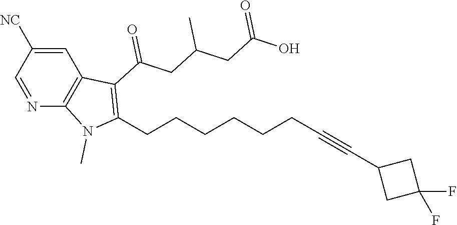 [SMILES: CC(CC(=O)O)CC(=O)c1c(CCCCCCC#CC2CC(F)(F)C2)n(C)c2ncc(C#N)cc12] | 484.3 | 1H NMR (400 MHz, DMSO-d6) δ 12.11 (s, 1H), 8.83 (d, J = 1.6 Hz, 1H), 8.73 (d, J = 1.6 Hz, 1H), 3.86 (s, 3H), 3.21 (t, J = 7.6 Hz, 2H), 3.09 (dd, J = 16.8, 5.6 Hz, 1H), 2.95-2.87 (m, 4H), 2.54-2.51 (m, 2H), 2.49-2.46 (m, 1H), 2.41 (dd, J = 15.6, 5.6 Hz, 1H), 2.21-2.15 (m, 3H), 1.65-1.56 (m, 2H), 1.50- 1.38 (m, 6H), 0.97 (d, J = 6.4 Hz, 3H). |
| I-323 | 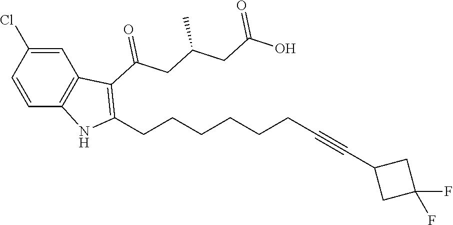 [SMILES: C[C@H](CC(=O)O)CC(=O)c1c(CCCCCCC#CC2CC(F)(F)C2)[nH]c2ccc(Cl)cc12] | 478.1 | 1H NMR (400 MHz, DMSO-d6) δ 7.96 (d, J = 1.6 Hz, 1H), 7.40 (d, J = 8.8 Hz, 1H), 7.15 (dd, J = 8.4 Hz, 2.0 Hz, 1H), 3.05 (t, J = 7.2 Hz, 2H), 2.98-2.86 (m, 4H), 2.76 (dd, J = 16.0 Hz, J = 7.6 Hz, 1H), 2.54- 2.50 (m, 2H), 2.46-2.41 (m, 1H), 2.31 (dd, J = 14.8 Hz, 6.0 Hz, 1H), 2.16-2.08 (m, 3H), 1.68-1.65 (m, 2H), 1.44-1.34 (m, 6H), 0.96 (d, J = 6.4 Hz, 3H). |
| I-324 | 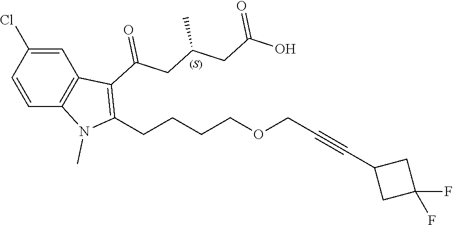 | 494.2 | 1H NMR (400 MHz, DMSO-d6) δ 12.08 (s, 1H), 7.96 (d, J = 1.6 Hz, 1H), 7.59 (d, J = 8.8 Hz, 1H), 7.25 (dd, J = 8.8, 2.0 Hz, 1H), 4.13 (d, J = 2.0 Hz, 2H), 3.76 (s, 3H), 3.48 (t, J = 5.6 Hz, 2H), 3.16 (t, J = 7.6 Hz, 2H), 3.02-2.86 (m, 5H), 2.63- 2.54 (m, 2H), 2.49-2.46 (m, 1H), 2.39 (dd, J = 15.2 Hz, 5.6 Hz, 1H), 2.17 (dd, J = 15.2 Hz, 8.0 Hz, 1H), 1.69-1.58 (m, 4H), 0.97 (d, J = 6.4 Hz, 3H). |
| I-325 | 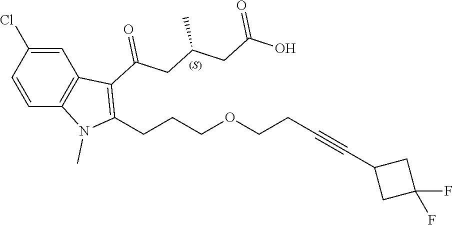 | 494.2 | 1H NMR (400 MHz, DMSO-d6) δ 12.08 (br., 1H), 7.97 (d, J = 1.6 Hz, 1H), 7.60 (d, J = 8.4 Hz, 1H), 7.26 (dd, J = 8.8 Hz, 2.0 Hz, 1H), 3.77 (s, 3H), 3.48 (t, J = 6.8 Hz, 4H), 3.18 (t, J = 7.2 Hz, 2H), 3.02-2.82 (m, 5H), 2.57-2.52 (m, 2H), 2.46- 2.33 (m, 4H), 2.16 (dd, J = 15.6 Hz, 8.4 Hz, 1H), 1.86-1.79 (m, 2H), 0.97 (d, J = 6.8 Hz, 3H). |
| I-326 | 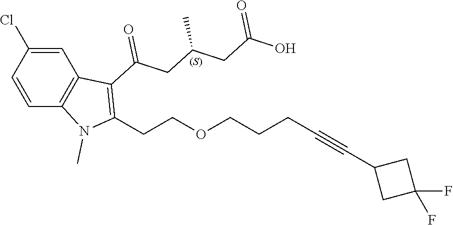 | 494.3 | 1H NMR (400 MHz, DMSO-d6) δ 12.11 (br., 1H), 7.95 (d, J = 1.6 Hz, 1H), 7.62 (d, J = 8.8 Hz, 1H), 7.27 (dd, J = 8.8, 2.0 Hz, 1H), 3.79 (s, 3H), 3.65 (t, J = 6.4 Hz, 2H), 3.41 (t, J = 6.0 Hz, 4H), 3.02 (dd, J = 16.4 Hz, 5.6 Hz, 1H), 2.91-2.85 (m, 4H), 2.53-2.50 (m, 2H), 2.48- 2.45 (m, 1H), 2.39 (dd, J = 15.2 Hz, 5.6 Hz, 1H), 2.20-2.11 (m, 3H), 1.62-1.55 (m, 2H), 0.97 (d, J = 6.8 Hz, 3H). |
| I-327 | 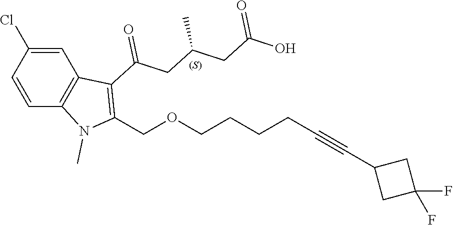 | 494.3 | 1H NMR (400 MHz, DMSO-d6) δ 8.06 (d, J = 1.6 Hz, 1H), 7.65 (d, J = 8.8 Hz, 1H), 7.32 (dd, J = 8.8 Hz, 2.0 Hz, 1H), 4.98 (s, 2H), 3.82 (s, 3H), 3.51 (t, J = 6.0 Hz, 2H), 3.05 (dd, J = 16.4 Hz, 6.0 Hz, 1H), 2.94-2.85 (m, 4H), 2.54-2.52 (m, 2H), 2.45-2.42 (m, 1H), 2.35 (dd, J = 15.6 Hz, 6.0 Hz, 1H), 2.17- 2.11 (m, 3H), 1.62-1.56 (m, 2H), 1.49-1.43 (m, 2H), 0.95 (d, J = 6.8 Hz, 3H). |
| I-328 | 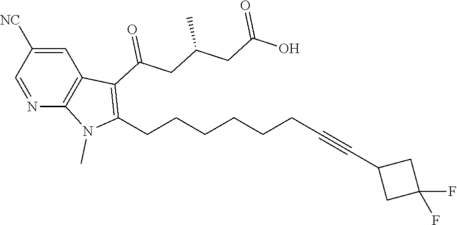 [SMILES: C[C@H](CC(=O)O)CC(=O)c1c(CCCCCCC#CC2CC(F)(F)C2)n(C)c2ncc(C#N)cc12] | 484.3 | 1H NMR (400 MHz, DMSO-d6) δ 12.14 (br., 1H), 8.84 (d, J = 1.6 Hz, 1H), 8.73 (d, J = 1.6 Hz, 1H), 3.86 (s, 3H), 3.21 (t, J = 7.6 Hz, 2H), 3.10 (dd, J = 17.2 Hz, 5.6 Hz, 1H), 2.95-2.87 (m, 4H), 2.57-2.52 (m, 1H), 2.49-2.37 (m, 3H), 2.21-2.14 (m, 3H), 1.64-1.55 (m, 2H), 1.48- 1.37 (m, 6H), 0.97 (d, J = 6.4 Hz, 3H). |
| I-329 | 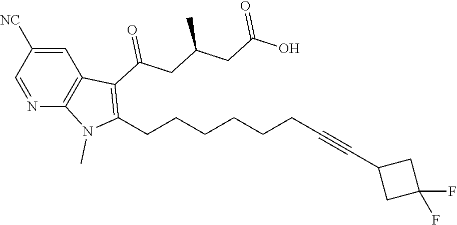 [SMILES: C[C@@H](CC(=O)O)CC(=O)c1c(CCCCCCC#CC2CC(F)(F)C2)n(C)c2ncc(C#N)cc12] | 484.3 | 1H NMR (400 MHz, DMSO-d6) δ 12.18 (br., 1H), 8.84 (d, J = 1.6 Hz, 1H), 8.73 (d, J = 1.6 Hz, 1H), 3.86 (s, 3H), 3.21 (t, J = 7.6 Hz, 2H), 3.10 (dd, J = 16.8 Hz, 5.6 Hz, 1H), 2.96-2.86 (m, 4H), 2.57-2.52 (m, 1H), 2.49-2.37 (m, 3H), 2.21-2.14 (m, 3H), 1.64-1.55 (m, 2H), 1.48- 1.37 (m, 6H), 0.97 (d, J = 6.4 Hz, 3H). |
| I-330 | 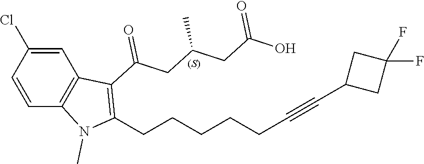 | 478.2 | 1H NMR (400 MHz, DMSO-d6) δ 12.11 (br, 1H), 7.96 (d, J = 1.2 Hz, 1H), 7.59 (d, J = 8.8 Hz, 1H), 7.24 (dd, J = 8.8 Hz, 1.6 Hz, 1H), 3.76 (s, 3H), 3.14 (t, J = 7.6 Hz, 2H), 3.02-2.82 (m, 5H), 2.53-2.51 (m, 1H), 2.49-2.44 (m, 2H), 2.39 (dd, J = 15.6 Hz, 5.6 Hz, 1H), 2.20- 2.14 (m, 3H), 1.63-1.47 (m, 6H), 0.98 (d, J = 6.4 Hz, 3H). |
| I-331 | 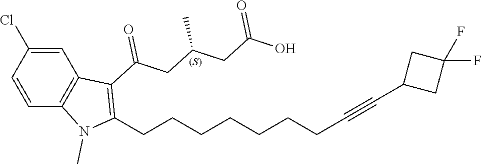 | 506.3 | 1H NMR (400 MHz, DMSO-d6) δ 12.16 (br., 1H), 7.96 (d, J = 2.0 Hz, 1H), 7.59 (d, J = 8.8 Hz, 1H), 7.25 (dd, J = 8.8 Hz, 2.0 Hz, 1H), 3.76 (s, 3H), 3.14 (t, J = 8.0 Hz, 2H), 3.01-2.81 (m, 5H), 2.55-2.52 (m, 2H), 2.48-2.36 (m, 2H), 2.20-2.14 (m, 3H), 1.59-1.52 (m, 2H), 1.42- 1.34 (m, 8H), 0.97 (d, J = 6.8 Hz, 3H). |
| I-332 | 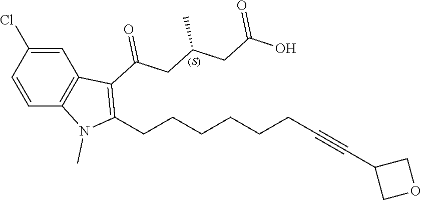 | 458.4 | 1H NMR (400 MHz, DMSO-d6) δ 12.12 (s, 1H), 7.95 (d, J = 2.0 Hz, 1H), 7.59 (d, J = 8.8 Hz, 1H), 7.25 (dd, J = 8.8 Hz, 2.0 Hz, 1H), 4.68 (dd, J = 8.4 Hz, 5.2 Hz, 2H), 4.45 (dd, J = 7.2 Hz, 5.2 Hz, 2H), 3.86- 3.81 (m, 1H), 3.76 (s, 3H), 3.14 (t, J = 7.2 Hz, 2H), 2.98 (dd, J = 16.4 Hz, 6.0 Hz, 1H), 2.84 (dd, J = 16.4 Hz, 7.2 Hz, 1H), 2.49-2.46 (m, 1H), 2.38 (dd, J = 15.2 Hz, 5.6 Hz, 1H), 2.22-2.14 (m, 3H), 1.61-1.52 (m, 2H), 1.50-1.39 (m, 6H), 0.97 (d, J = 6.4 Hz, 3H). |
| I-333 | 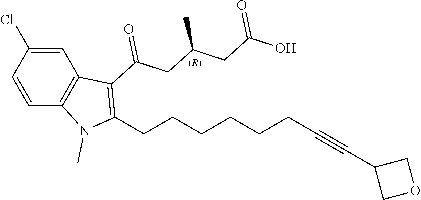 | 458.3 | 1H NMR (400 MHz, DMSO-d6) δ 12.10 (s, 1H), 7.96 (d, J = 2.0 Hz, 1H), 7.59 (d, J = 8.8 Hz, 1H), 7.25 (dd, J = 8.4 Hz, 2.0 Hz, 1H), 4.68 (dd, J = 8.4 Hz, 5.2 Hz, 2H), 4.45 (dd, J = 7.2 Hz, 5.6 Hz, 2H), 3.87- 3.81 (m, 1H), 3.76 (s, 3H), 3.14 (t, J = 7.6 Hz, 2H), 2.98 (dd, J = 16.4 Hz, 6.0 Hz, 1H), 2.85 (dd, J = 16.4 Hz, 7.6 Hz, 1H), 2.49-2.46 (m, 1H), 2.39 (dd, J = 15.6 Hz, 6.0 Hz, 1H), 2.22-2.14 (m, 3H), 1.60-1.53 (m, 2H), 1.50-1.38 (m, 6H), 0.97 (d, J = 6.4 Hz, 3H). |
| I-334 | 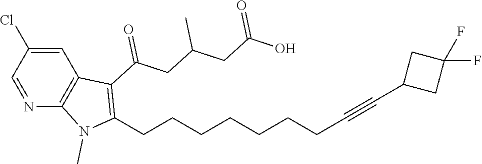 [SMILES: CC(CC(=O)O)CC(=O)c1c(CCCCCCCC#CC2CC(F)(F)C2)n(C)c2ncc(Cl)cc12] | 507.3 | 1H NMR (400 MHz, DMSO-d6) δ 12.08 (br. 1H), 8.35 (d, J = 2.4 Hz, 1H), 8.30 (d, J = 2.4 Hz, 1H), 3.79 (s, 3H), 3.16 (t, J = 7.6 Hz, 2H), 3.02 (dd, J = 16.8 Hz, 5.6 Hz, 1H), 2.91-2.81 (m, 4H), 2.43-2.34 (m, 4H), 2.18-2.12 (m, 3H), 1.57-1.53 (m, 2H), 1.40-1.32 (m, 8H), 0.94 (d, J = 6.4 Hz, 3H). |
| I-335 | 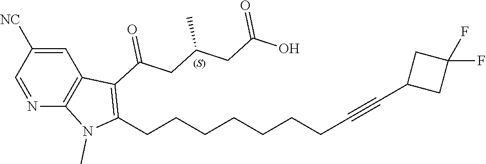 | 498.3 | 1H NMR (400 MHz, DMSO-d6) δ 12.10 (br., 1H), 8.83 (d, J = 2.0 Hz, 1H), 8.73 (d, J = 2.0 Hz, 1H), 3.86 (s, 3H), 3.21 (t, J = 8.0 Hz, 2H), 3.10 (dd, J = 16.8 Hz, 5.6 Hz, 1H), 2.96-2.85 (m, 4H), 2.57-2.51 (m, 1H), 2.49-2.37 (m, 3H), 2.22-2.14 (m, 3H), 1.65-1.54 (m, 2H), 1.46- 1.29 (m, 8H), 0.97 (d, J = 14.8 Hz, 3H). |
| I-336 | 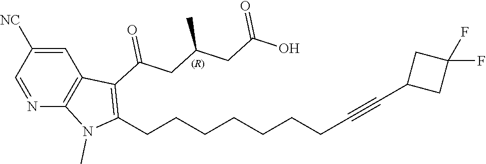 | 498.3 | 1H NMR (400 MHz, DMSO-d6) δ 12.15 (s, 1H), 8.83 (d, J = 2.0 Hz, 1H), 8.73 (d, J = 2.0 Hz, 1H), 3.86 (s, 3H), 3.21 (t, J = 8.0 Hz, 2H), 3.10 (dd, J = 16.8 Hz, 5.6 Hz, 1H), 2.97-2.82 (m, 4H), 2.57-2.51 (m, 1H), 2.49-2.37 (m, 3H), 2.22-2.11 (m, 3H), 1.65-1.54 (m, 2H), 1.46- 1.29 (m, 8H), 0.97 (d, J = 6.4 Hz, 3H). |
| I-337 | 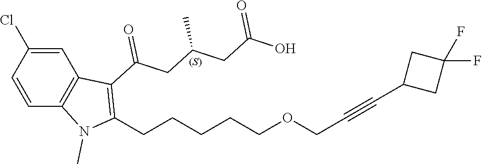 | 508.3 | 1H NMR (400 MHz, DMSO-d6) δ 12.30 (br., 1H), 7.95 (d, J = 1.6 Hz, 1H), 7.59 (d, J = 8.8 Hz, 1H), 7.25 (dd, J = 8.8 Hz, 2.0 Hz, 1H), 4.11 (d, J = 1.6 Hz, 2H), 3.76 (s, 3H), 3.43 (t, J = 6.4 Hz, 2H), 3.14 (t, J = 7.2 Hz, 2H), 3.02-2.84 (m, 5H), 2.60-2.52 (m, 2H), 2.50-2.44 (m, 1H), 2.36 (dd, J = 15.2 Hz, 5.2 Hz, 1H), 2.15 (dd, J = 15.2 Hz, 8.0 Hz, 1H), 1.59-1.54 (m, 4H), 1.51-1.44 (m, 2H), 0.97 (d, J = 6.4 Hz, 3H). |

TABLE 5a
Additional Characterization Data for Additional Exemplary Compounds

| Compound | Rt (Min) | Rt (Min) |
|---|---|---|
| No. | (LCMS) | (HPLC) |
| I-319 | 2.107 | 10.738 |
| I-320 | 2.124 | 10.805 |
| I-321 | 1.961 | 9.805 |
| I-322 | 2.006 | 10.098 |
| I-323 | 2.008 | 8.342 |
| I-324 | 1.984 | 9.990 |
| I-325 | 1.990 | 10.048 |
| I-326 | 2.013 | 10.199 |
| I-327 | 1.796 | 8.538 |
| I-328 | 2.005 | 10.176 |
| I-329 | 2.005 | 10.175 |
| I-330 | 2.046 | 10.513 |
| I-331 | 2.150 | 11.129 |
| I-332 | 1.786 | 9.327 |
| I-333 | 1.776 | 9.325 |
| I-334 | 2.166 | 11.236 |
| I-335 | 2.074 | 10.572 |
| I-336 | 2.075 | 10.559 |
| I-337 | 2.021 | 10.252 |

### Example 5: Synthesis of Compound I-139 PGP181,C3

### Synthetic Scheme for Compound 1-139

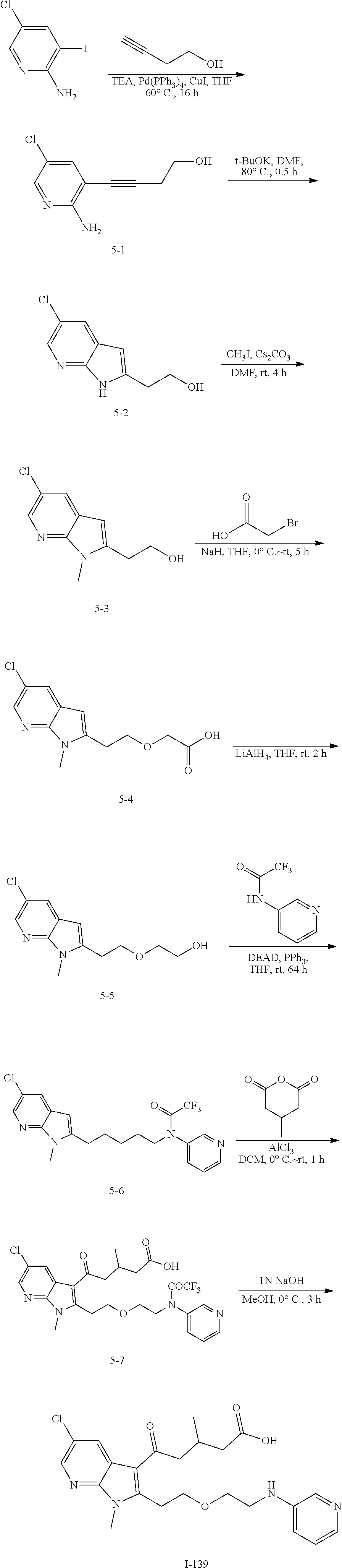

### 5.1 The Synthesis of Intermediate 5-1

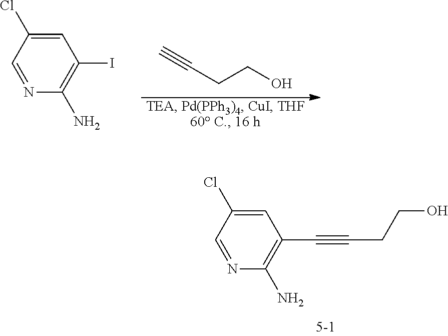

To a stirred solution of 5-chloro-3-iodopyridin-2-amine (15.0 g, 58.8 mmol) in THF (70 mL) was added but-3-yn-1-ol (4.5 g, 64.8 mmol), Pd(PPh₃)₂Cl₂ (750 mg), CuI (1.5 g) and TEA (18.0 g, 176.7 mmol). The reaction mixture was stirred at 60° C. for 16 h under nitrogen. The mixture was filtered through a pad of diatomite, then H₂O (250 mL) and EA (250 mL) were added and the organic phases was collected and evaporated under reduced pressure to give 5-1 (16.8 g, crude) as yellow oil.

### 5.2 The Synthesis of Intermediate 5-2

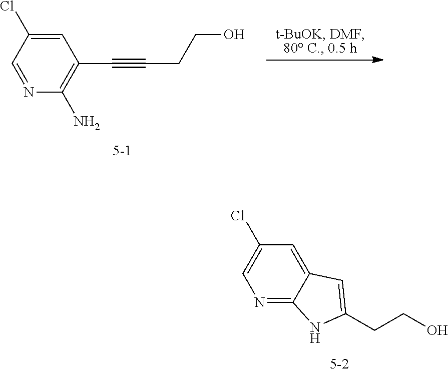 [SMILES: Nc1ncc(Cl)cc1C#CCCO ; "t-BuOK, DMF,80° C., 0.5 h" ; OCCc1cc2cc(Cl)cnc2[nH]1]

To a stirred solution of 5-1 (14.0 g, 72.1 mmol) in DMF (100 mL) was added t-BuOK (11.3 g, 100.9 mmol). The reaction mixture was stirred at 80° C. for 0.5 h under nitrogen. The mixture was filtered through a pad of diatomite and purified by prep-HPLC (MeCN/water: 5% to 95%) to give 5-2 (7.3 g, yield: 52%) as a yellow solid.

### 5.3 The Synthesis of Intermediate 5-3

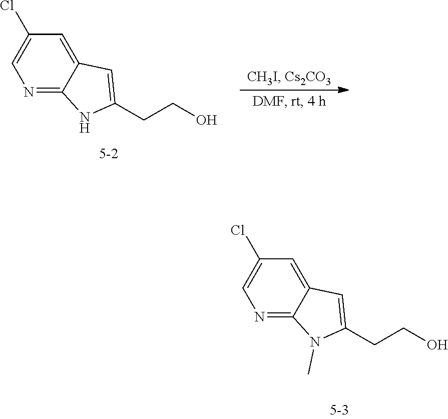

To a stirred solution of 5-2 (7.3 g, 37.2 mmol) in DMF (40 mL) was added Mel (5.8 g, 40.9 mmol) and Cs₂CO₃ (13.3 g, 40.9 mmol). The reaction mixture was stirred at rt for 4 h until the reaction was complete (by LCMS), then H₂O (100 mL) and EA (100 mL) were added and the organic phases was collected and evaporated under reduced pressure. The mixture was purified by silica gel chromatograph column (EA/PE: 0:1 to 1:0) to give 5-3 (3.0 g, yield: 38%) as a yellow solid.

### 5.4 The Synthesis of Intermediate 5-4

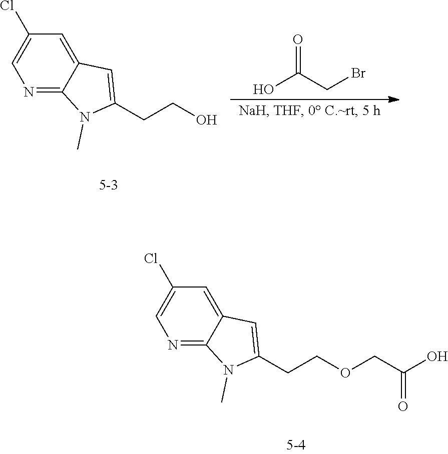

To a stirred solution of 5-3 (2.5 g, 11.9 mmol) and 2-bromoacetic acid (3.3 g, 23.7 mmol) in DMF (25 mL) was added NaH (60% dispersion in mineral oil, 1.2 g, 29.7 mmol) at 0° C. The reaction mixture was stirred at rt for 1 hour, then H₂O (75 mL) and EA (75 mL) were added and the organic phases was washed with brine, evaporated under reduced pressure to give 5-4 (1.5 g, yield: 47%) as yellow oil.

### 5.5 The Synthesis of Intermediate 5-5

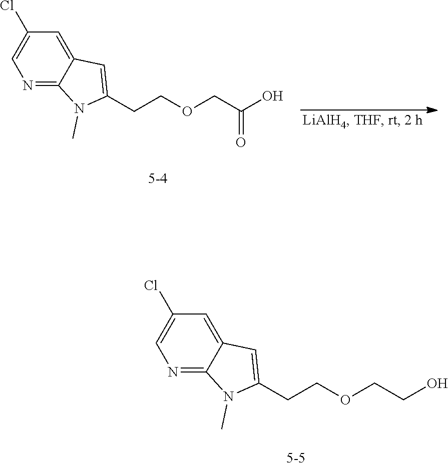 [SMILES: Cn1c(CCOCC(=O)O)cc2cc(Cl)cnc21 ; "LiAlH4, THF, rt, 2 h" ; Cn1c(CCOCCO)cc2cc(Cl)cnc21]

To a stirred solution of 5-4 (1.5 g, 5.6 mmol) in THF (15 mL) was added LiAlH₄ (848 mg, 22.3 mmol) at 0° C. The reaction mixture was stirred at rt for 2 h until the reaction was complete (by LCMS), then H₂O (1 mL) and NaOH (15% in water, 1 mL) was added dropwise at 0° C. successively to quench the reaction. The mixture was filtered and the filtrate was dried over anhydrous Na₂SO₄ and evaporated under reduced pressure to afford the crude product 5-5 (1.1 g, yield: 77%) as yellow oil, which was used to the next step directly.

### 5.6 The Synthesis of Intermediate 5-6

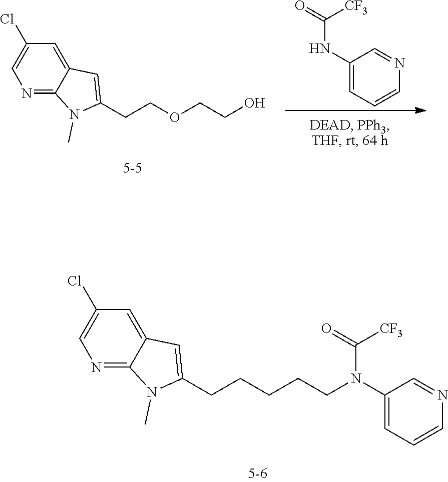

To a stirred solution of 2,2,2-trifluoro-N-(pyridin-3-yl)acetamide (1.6 g, 8.6 mmol) in THF (15 mL) was added DEAD (1.5 g, 8.6 mmol), PPh₃ (2.3 g, 8.6 mmol) and 5-5 (1.1 g, 4.3 mmol) at 0° C. The reaction mixture was stirred at rt for 64 hours until the reaction was complete (by LCMS). The reaction was quenched by adding water and extracted with EtOAc. The organic layer was combined, washed with brine and dried over anhydrous Na₂SO₄. The solvent was evaporated under reduced pressure then purified by silica gel chromatograph column (DCM: MeOH=1: 0 to 10:1) to afford 5-6 (290 mg, yield: 16%) as yellow oil.

### 5.7 The Synthesis of Intermediate 5-7

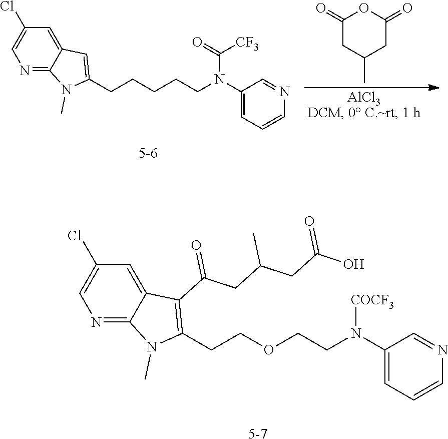

To a stirred solution of 5-6 (200 mg, 0.47 mmol) in dichloromethane (5 mL) was added AlCl₃ (312 mg, 2.3 mmol) at 0° C. After 10 min, 4-methyldihydro-2H-pyran-2,6(3H)-dione (300 mg, 2.3 mmol) in dichloromethane (1.0 mL) was added dropwise and the reaction mixture was stirred at room temperature for another 1 h. The reaction was quenched by adding water (1 mL) and extracted with EtOAc (15 mL×3). The organic layer was combined, washed with brine and dried over Na₂SO₄. The solvent was evaporated under reduced pressure and purified by prep-HPLC to afford 5-7 (45 mg, yield: 17%) as yellow oil.

### 5.8 The Synthesis of Compound I-139

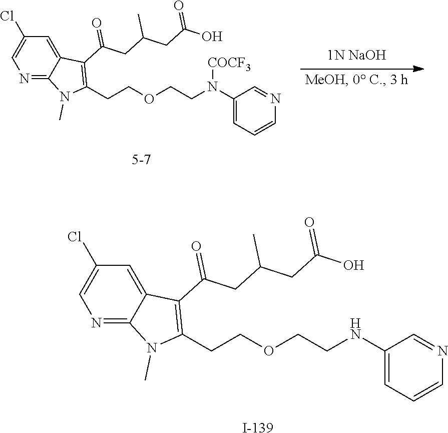

To a stirred solution of 5-7 (40 mg crude, 0.072 mmol) in MeOH (2 mL) was added NaOH (0.2 mL 1 M in water, 0.2 mmol). The reaction mixture was stirred at 0° C. for 3 h until the reaction was complete (by LCMS). Saturated NH₄Cl solution was added at 0° C. and the mixture was acidified with HCl (1.0 M) to pH ˜4.0 then extracted with EtOAc, the combined organic layer was washed with brine, dried over anhydrous Na₂SO₄ and evaporated under reduced pressure. The crude was purified by Prep-HPLC to afford 1-139 (6 mg, yield: 18%) as a white solid.

¹H NMR (400 MHz, CD₃OD) δ 8.35 (d, J=2.0 Hz, 1H), 8.28 (d, J=2.0 Hz, 1H), 7.89 (t, J=1.6 Hz, 1H), 7.73 (d, J=5.6 Hz, 1H), 7.28-7.25 (m, 1H), 7.22-7.18 (m, 1H), 4.50-4.47 (m, 2H), 3.89-3.84 (m, 4H), 3.77 (s, 3H), 3.53-3.45 (m, 2H), 3.08 (dd, J=15.6, 5.2 Hz, 1H), 2.78 (dd, J=15.6, 8.4 Hz, 1H), 2.62-2.52 (m, 1H), 2.24-2.20 (m, 2H), 1.05 (d, J=6.4 Hz, 3H).

LC-MS m/z: 458.9 [M+H]⁺.

Additional exemplary compounds were prepared following methods substantially similar to the method described above and herein. Data for these compounds are provided in Table Table 6below.

TABLE 6
Characterization Data for Additional Exemplary Compounds

| Com- | | | |
|---|---|---|---|
| pound | | | |
| No. | Chemical Structure | M + 1 | ¹H NMR (400 MHz) |
| I-79 | 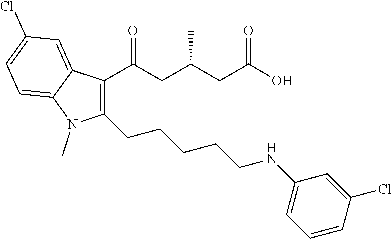 [SMILES: C[C@H](CC(=O)O)CC(=O)c1c(CCCCCNc2cccc(Cl)c2)n(C)c2ccc(Cl)cc12] | 489.0 | ¹H NMR (400 MHz, DMSO-d₆) δ 12.04 (s, 1H), 7.95 (d, J = 1.6 Hz, 1H), 7.60 (d, J = 8.8 Hz, 1H), 7.25 (dd, J = 8.8, 1.6 Hz, 1H), 7.04 (t, J = 8.0 Hz, 1H), 6.54 (t, J = 2.0 Hz, 1H), 6.51-6.47 (m, 2H), 5.93 (s, 1H), 3.77 (s, 3H), 3.16 (t, J = 7.2 Hz, 2H), 3.02-2.96 (m, 3H), 2.85 (dd, J = 16.4, 7.6 Hz, 1H), 2.55-2.52 (m, 1H), 2.39 (dd, J = 15.6, 5.6 Hz, 1H), 2.17 (dd, J = 15.6, 8.0 Hz, 1H), 1.65-1.45 (m, 6H), 0.97 (d, J = 6.4 Hz, 3H). |
| I-80 | 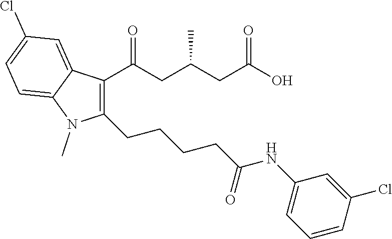 [SMILES: C[C@H](CC(=O)O)CC(=O)c1c(CCCCC(=O)Nc2cccc(Cl)c2)n(C)c2ccc(Cl)cc12] | 502.9 | ¹H NMR (400 MHz, CDCl₃) δ 12.07 (s, 1H), 10.11 (s, 1H), 7.96 (d, J = 2.0 Hz, 1H), 7.83 (t, J = 2.0 Hz, 1H), 7.59 (d, J = 8.8, 1H), 7.45-7.41 (m, 1H), 7.31 (t, J = 8.0 Hz, 1H), 7.25 (dd, J = 8.8, 2.0 Hz, 1H), 7.09-7.07 (m, 1H), 3.77 (s, 3H), 3.19 (t, J = 8.0 Hz, 2H), 3.00 (dd, J = 16.4, 5.6 Hz, 1H), 2.86 (dd, J = 16.4, 7.6 Hz, 1H), 2.56- 2.47 (m, 1H), 2.43-2.37 (m, 3H), 2.18 (dd, J = 15.2, 8.0 Hz, 1H), 1.77-1.60 (m, 4H), 0.97 (d, J = 6.8 Hz, 3H). |
| I-82 | 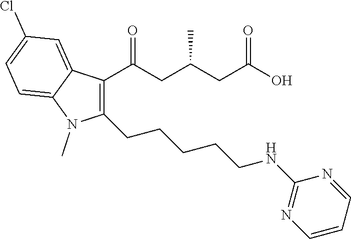 [SMILES: C[C@H](CC(=O)O)CC(=O)c1c(CCCCCNc2ncccn2)n(C)c2ccc(Cl)cc12] | 457.0 | ¹H NMR (400 MHz, CDCl₃) δ 8.28 (br, 2 H), 7.84 (s, 1 H), 7.20 (s, 2 H), 6.50 (t, J = 5.2 Hz, 2H), 3.71 (s, 3H), 3.44-3.31 (m, 3H), 3.18-3.00 (m, 3H), 2.79 (br, 1H), 2.56 (dd, J = 15.6, 8.0 Hz, 1H), 2.41 (dd, J = 15.6, 8.0 Hz, 1H), 1.72-1.62 (m, 6H), 1.17 (d, J = 6.8 Hz, 3H). |
| I-124 | 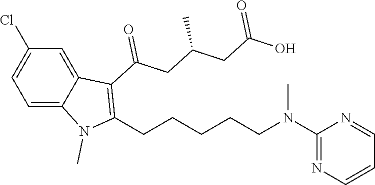 [SMILES: C[C@H](CC(=O)O)CC(=O)c1c(CCCCCN(C)c2ncccn2)n(C)c2ccc(Cl)cc12] | 471.0 | ¹H NMR (400 MHz, CDCl₃) δ 8.24 (d, J = 4.8 Hz, 2H), 7.93 (s, 1H), 7.16-7.14 (m, 2H), 6.39 (t, J = 4.8 Hz, 1H), 3.62 (s, 3H), 3.60-3.47 (m, 2H), 3.11-3.09 (m, 2H), 3.07 (s, 3H), 2.96 (dd, J = 16.0, 6.4 Hz, 1H), 2.85 (dd, J = 16.0, 7.2 Hz, 1H), 2.68-2.63 (m, 1H), 2.44 (dd, J = 15.2, 6.4 Hz, 1H), 2.33 (dd, J = 14.8, 6.4 Hz, 1H), 1.64-1.56 (m, 4H), 1.46-1.40 (m, 2H), 1.09 (d, J = 6.4 Hz, 3H). |
| I-126 | 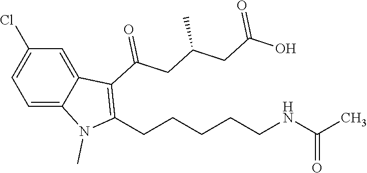 [SMILES: CC(=O)NCCCCCc1c(C(=O)C[C@H](C)CC(=O)O)c2cc(Cl)ccc2n1C] | 421.0 | ¹H NMR (400 MHz, DMSO-d₆) δ 7.96 (d, J = 2.0 Hz, 1H), 7.81 (s, 1H), 7.60 (d, J = 8.8 Hz, 1H), 7.25 (dd, J = 8.0, 2.0 Hz, 1H), 3.76 (s, 3H), 3.13 (t, J = 7.6 Hz, 2H), 3.06- 2.96 (m, 3H), 2.85 (dd, J = 16.4, 7.6 Hz, 1H), 2.50-2.47 (m, 1H), 2.39 (dd, J = 15.6, 5.6 Hz, 1H), 2.18 (dd, J = 15.6, 8.8 Hz, 1H), 1.78 (s, 3H), 1.60-1.54 (m, 2H), 1.47- 1.40 (m, 4H), 0.97 (d, J = 6.8 Hz, 3H). |
| I-127 | 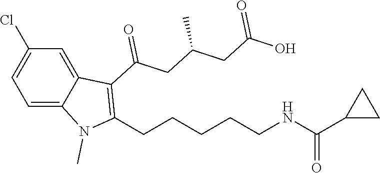 [SMILES: C[C@H](CC(=O)O)CC(=O)c1c(CCCCCNC(=O)C2CC2)n(C)c2ccc(Cl)cc12] | 447.0 | ¹H NMR (400 MHz, DMSO-d₆) δ 8.03 (t, J = 5.6 Hz, 1H), 7.96 (d, J = 2.0 Hz, 1H), 7.59 (d, J = 8.8 Hz, 1H), 7.25 (dd, J = 8.8 Hz, J = 2.0 Hz, 1H), 3.76 (s, 3H), 3.16-3.05 (m, 4H), 2.99 (dd, J = 16.8, 6.0 Hz, 1H), 2.85 (dd, J = 16.8, 7.6 Hz, 1H), 2.55-2.52 (m, 1H), 2.39 (dd, J = 15.2, 5.6 Hz, 1H), 2.18 (dd, J = 15.2, 8.0 Hz, 1H), 1.59-1.41 (m, 7H), 0.98 (d, J = 6.8 Hz, 3H), 0.66- 0.58 (m, 4H). |
| I-128 | 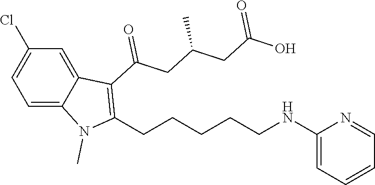 [SMILES: C[C@H](CC(=O)O)CC(=O)c1c(CCCCCNc2ccccn2)n(C)c2ccc(Cl)cc12] | 456.0 | ¹H NMR (400 MHz, DMSO-d₆) δ 7.96 (d, J = 2.0 Hz, 1H), 7.93 (dd, J = 8.0, 4.8 Hz, 1H), 7.59 (d, J = 8.4 Hz, 1H), 7.36-7.32 (m, 1H), 7.25 (dd, J = 8.8, 2.0 Hz, 1H), 6.57-6.48 (m, 1H), 6.45-6.42 (m, 2H), 3.76 (s, 3H), 3.25-3.20 (m, 2H), 3.17-3.13 (m, 2H), 2.99 (dd, J = 16.4, 6.0 Hz, 1H), 2.85 (dd, J = 16.4, 7.6 Hz, 1H), 2.48-2.44 (m, 1H), 2.39 (dd, J = 15.6, 6.0 Hz, 1H), 2.17 (dd, J = 15.6, 8.0 Hz, 1H), 1.62-1.55 (m, 4H), 1.51-1.48 (m, 2H), 0.97 (d, J = 6.8 Hz, 3H). |
| I-129 | 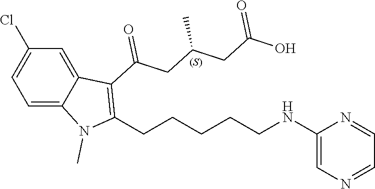 | 457.1 | ¹H NMR (400 MHz, DMSO-d₆) δ 8.09 (s, 1H), 8.00 (d, J = 1.2 Hz, 1H), 7.58 (d, J = 8.4 Hz, 1H), 7.43 (d, J = 4.4 Hz, 1H), 7.24 (dd, J = 8.8, 2.0 Hz, 1H), 7.08 (d, J = 4.4 Hz, 1H), 4.35-4.23 (br, 1H), 3.87 (t, J = 6.8 Hz, 2H), 3.76 (s, 3H), 3.14 (t, J = 8.0 Hz, 2H), 3.02 (dd, J = 16.4, 4.8 Hz, 1H), 2.80 (dd, J = 16.0, 7.6 Hz, 1H), 2.45-2.42 (m, 1H), 2.29 (dd, J = 15.2, 6.0 Hz, 1H), 2.14 (dd, J = 15.2, 7.2 Hz, 1H), 1.76-1.68 (m, 2H), 1.63-1.58 (m, 2H), 1.48-1.43 (m, 2H), 0.97 (d, J = 6.4 Hz, 3H). |
| I-134 | 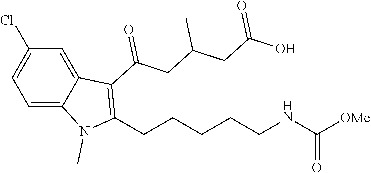 [SMILES: COC(=O)NCCCCCc1c(C(=O)CC(C)CC(=O)O)c2cc(Cl)ccc2n1C] | 436.9 | ¹H NMR (400 MHz, DMSO-d₆) δ 12.02 (br, 1H), 7.95 (d, J = 1.6 Hz, 1H), 7.59 (d, J = 8.8 Hz, 1H), 7.24 (dd, J = 8.8, 2.0 Hz, 1H), 7.12 (t, J = 5.2 Hz, 1H), 3.76 (s, 3H), 3.50 (s, 3H), 3.13 (t, J = 8.0 Hz, 2H), 3.02- 2.96 (m, 3H), 2.83 (dd, J = 16.4, 7.6 Hz, 1H), 2.54-2.50 (m, 1H), 2.36 (dd, J = 15.4, 5.6 Hz, 1H), 2.15 (dd, J = 15.2, 8.0 Hz, 1H), 1.60-1.54 (m, 2H), 1.49-1.39 (m, 4H), 0.97 (d, J = 6.4 Hz, 3H). |
| I-137 | 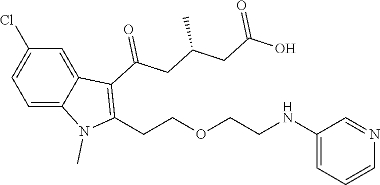 [SMILES: C[C@H](CC(=O)O)CC(=O)c1c(CCOCCNc2cccnc2)n(C)c2ccc(Cl)cc12] | 458.2 | ¹H NMR (400 MHz, DMSO-d₆) δ 8.26 (s, 1H), 7.95 (d, J = 1.6 Hz, 1H), 7.93-7.92 (m, 1H), 7.57 (d, J = 8.8 Hz, 1H), 7.44- 7.43 (m, 2H), 7.26 (dd, J = 8.4, 1.6 Hz, 1H), 6.85 (s, 2H), 4.54 (t, J = 4.4 Hz, 2H), 3.84 (t, J = 4.4 Hz, 2H), 3.71-3.68 (m, 5H), 3.47-3.40 (m, 1H), 3.36-3.29 (m, 1H), 3.07 (dd, J = 16.0, 5.2 Hz, 1H), 2.77-2.71 (m, 1H), 2.47-2.40 (m, 1H), 2.20 (dd, J = 14.8, 6.4 Hz, 1H), 2.08 (dd, J = 14.8, 7.2 Hz, 1H), 0.94 (d, J = 6.8 Hz, 3H). |
| I-140 | 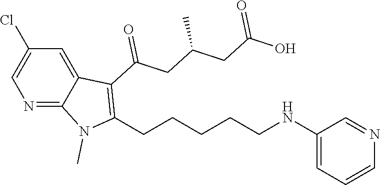 [SMILES: C[C@H](CC(=O)O)CC(=O)c1c(CCCCCNc2cccnc2)n(C)c2ncc(Cl)cc12] | 457.0 | ¹H NMR (400 MHz, DMSO-d₆) δ 9.12 (s, 1H), 8.44 (d, J = 2.0 Hz, 1H), 8.30 (d, J = 2.0 Hz, 1H), 8.05 (d, J = 5.6 Hz, 1H), 7.61- 7.57 (m, 1H), 7.50 (s, 2H), 7.45 (dd, J = 8.4, 1.6 Hz, 1H), 4.38 (t, J = 7.2 Hz, 2H), 3.80 (s, 3H), 3.55-3.42 (m, 1H), 3.36-3.32 (m, 1H), 3.16-3.05 (m, 1H), 2.59-2.53 (m, 1H), 2.42-2.32 (m, 1H), 2.10-1.88 (m, 4H), 1.72-1.58 (m, 2H), 1.47-1.40 (m, 2H), 0.87 (d, J = 6.8 Hz, 3H). |
| I-141 | 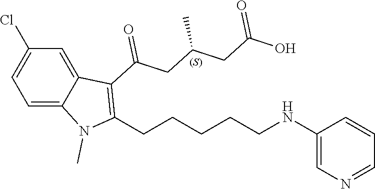 | 456.0 | ¹H NMR (400 MHz, DMSO-d₆) δ 9.25 (s, 1H), 8.08 (d, J = 2.0 Hz, 1H), 8.03 (d, J = 5.6 Hz, 1H), 7.60-7.53 (m, 4H), 7.44 (dd, J = 8.8, 2.0 Hz, 1H), 7.22 (dd, J = 8.8, 2.0 Hz, 1H), 4.38 (t, J = 6.8 Hz, 2H), 3.75 (s, 3H), 3.55-3.47 (m, 1H), 3.39-3.38 (m, 1H), 3.09-3.01 (m, 1H), 2.55-2.51 (m, 1H), 2.38-2.33 (m, 1H), 2.12-2.07 (m, 1H), 1.98 (dd, J= 14.8, 5.6 Hz, 2H), 1.89 (dd, J = 14.8, 8.4 Hz, 1H), 1.66-1.57 (m, 2H), 1.49-1.42 (m, 2H), 0.86 (d, J = 6.8 Hz, 3H). |
| I-142 | 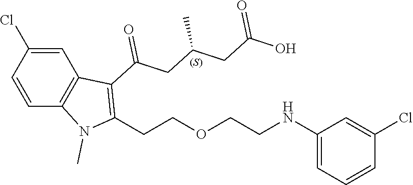 | 491.2 | ¹H NMR (400 MHz, DMSO-d₆) δ 12.05 (s, 1H), 7.94 (s, 1H), 7.60 (d, J = 8.8 Hz, 1H), 7.26 (dd, J = 8.8, 2.0 Hz, 1H), 7.02 (t, J = 8.0 Hz, 1H), 6.54 (t, J = 2.0 Hz, 1H), 6.51- 6.45 (m, 2H), 5.83 (s, 1H), 3.78 (s, 3H), 3.71 (t, J = 6.4 Hz, 2H), 3.52 (t, J = 5.6 Hz, 2H), 3.42 (t, J = 6.4 Hz, 2H), 3.13 (d, J = 5.2 Hz, 2H), 3.02 (dd, J = 16.4, 6.0 Hz, 1H), 2.88 (dd, J = 16.4, 7.6 Hz, 1H), 2.53- 2.50 (m, 1H), 2.39 (dd, J = 15.4, 5.6 Hz, 1H), 2.17 (dd, J = 15.4, 8.0 Hz, 1H), 0.97 (d, J = 6.8 Hz, 3H). |
| I-145 | 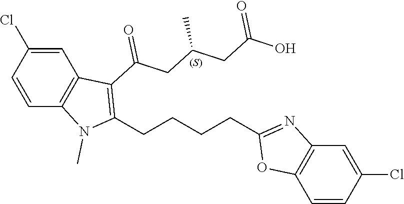 | 501.1 | ¹H NMR (400 MHz, CDCl₃) δ 7.89 (d, J = 0.8 Hz, 1H), 7.64 (d, J = 2.0 Hz, 1H), 7.38 (d, J = 8.8 Hz, 1H), 7.28 (d, J = 2.0 Hz, 1H), 7.23-7.20 (m, 2H), 3.71 (s, 3H), 3.36-3.24 (m, 1H), 3.29-3.24 (m, 1H), 3.06-2.96 (m, 4H), 2.80-2.75 (m, 1H), 2.51-2.39 (m, 2H), 2.07-2.02 (m, 2H), 1.82-1.73 (m, 2H), 1.17 (d, J = 6.8 Hz, 3H). |
| I-146 | 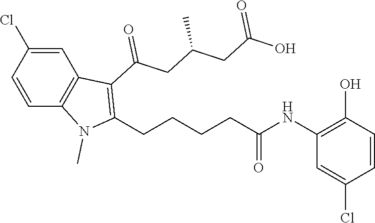 [SMILES: C[C@H](CC(=O)O)CC(=O)c1c(CCCCC(=O)Nc2cc(Cl)ccc2O)n(C)c2ccc(Cl)cc12] | 519.2 | ¹H NMR (400 MHz, CDCl₃) δ 8.90 (s, 1H), 7.83 (d, J = 1.2 Hz, 1H), 7.28-7.27 (m, 1H), 7.25-7.23 (m, 1H), 7.19 (d, J = 2.4 Hz, 1H), 7.01 (dd, J = 8.4, 2.0 Hz, 1H), 6.89 (d, J = 8.8 Hz, 1H), 3.73 (s, 3H), 3.24-3.20 (m, 2H), 3.05 (dd, J = 13.2, 6.4 Hz, 2H), 2.80-2.75 (m, 1H), 2.67 (t, J = 6.4 Hz, 2H), 2.52 (dd, J = 15.2, 6.0 Hz, 1H), 2.38 (dd, J = 15.6, 7.2 Hz, 1H), 1.94- 1.90 (m, 2H), 1.74-1.65 (m, 2H), 1.13 (d, J = 6.4 Hz, 3H). |

### Example 6: Synthesis of Compound I-147

### Synthetic Scheme for Compound 1-147

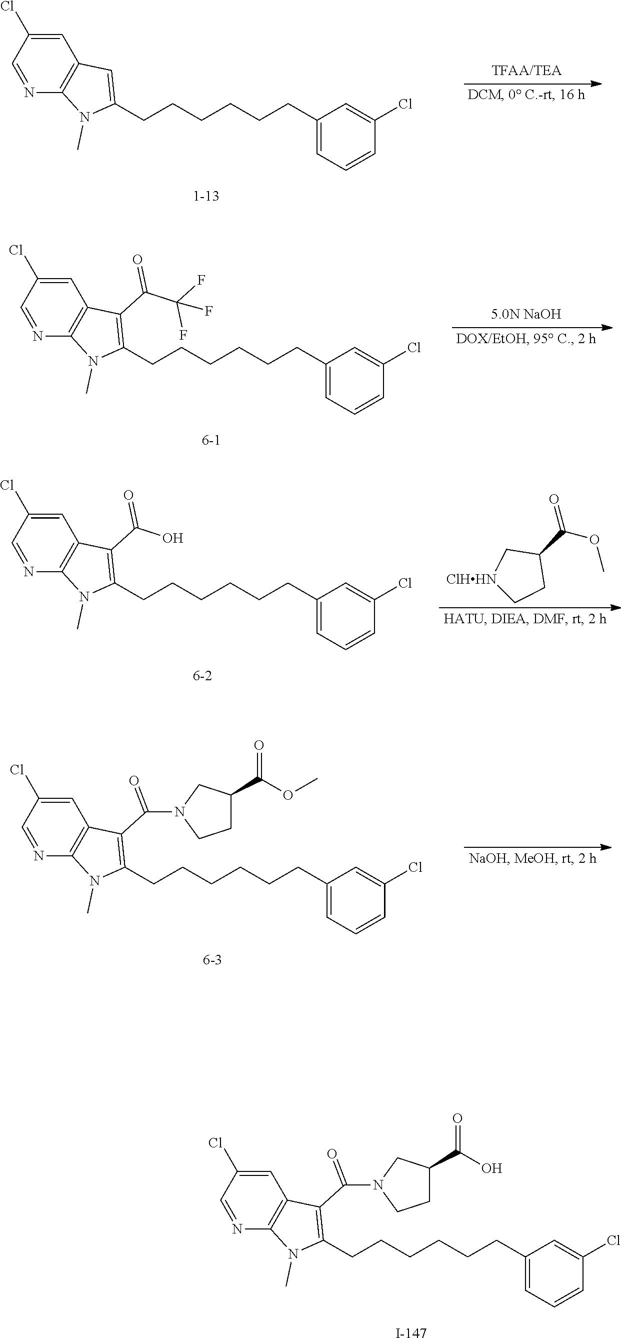

### 6.1 The Synthesis of Intermediate 6-1

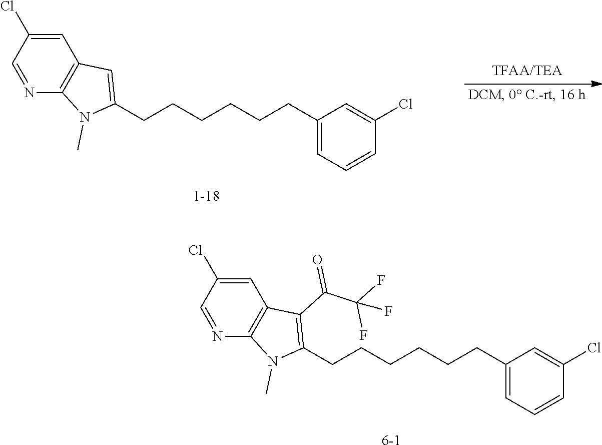 [SMILES: Cn1c(CCCCCCc2cccc(Cl)c2)cc2cc(Cl)cnc21 ; "DCM, 0° C.-rt, 16 h" ; "TFAA/TEA" ; "1-18" ; Cn1c(CCCCCCc2cccc(Cl)c2)c(C(=O)C(F)(F)F)c2cc(Cl)cnc21 ; "6-1"]

To a solution of 1-18 (200 mg, 0.56 mmol) in DCM (10 mL) was added TEA (565 mg, 5.6 mmol) at 0° C., then TFAA (1.17 g, 5.6 mmol) was added dropwise. The reaction mixture was allowed to warm to room temperature and stirred for 16 h. Saturated NaHCO₃ solution was added at 0° C. and the mixture was extracted with DCM (10 mL×2), the combined organic layer was washed with brine, dried over anhydrous Na₂SO₄ and evaporated under reduced pressure to afford the crude product 6-1 (230 mg, 91%) as yellow oil, which was used to the next step directly.

### 6.2 The Synthesis of Intermediate 6-2

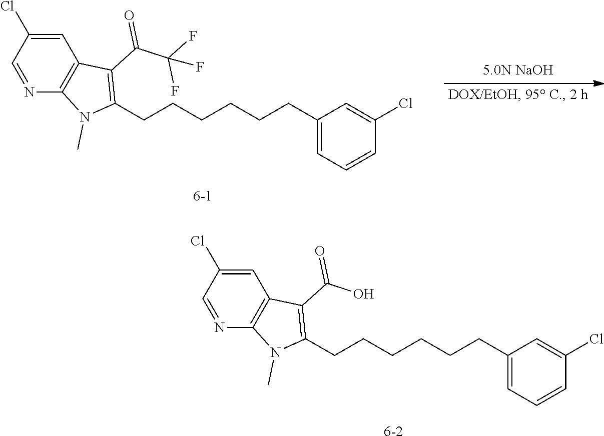

To a solution of 6-1 (230 mg, 0.5 mmol) in dioxane (9 mL) and EtOH (1 mL) was added NaOH (5.0 M, 1 mL, 5 mmol) at 0° C. Then the reaction mixture was allowed to warm to room temperature and stirred at 95° C. for 2 h. The mixture was acidified with HCl (3.0 M) to pH ˜3.0 then extracted with EtOAc (20 mL×3), the combined organic layer was washed with brine, dried over anhydrous Na₂SO₄ and evaporated under reduced pressure. The crude was purified by silica gel chromatography (14% MeOH/DCM) to afford 6-2 (200 mg, 98%) as a yellow solid.

### 6.3 The Synthesis of Intermediate 6-3

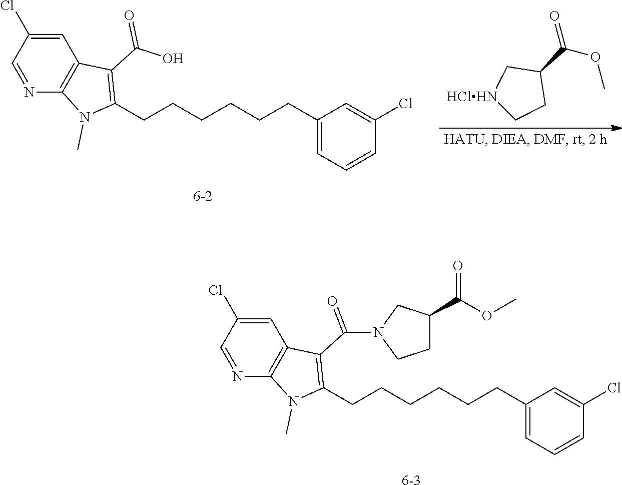

To a solution of 6-2 (200 mg, 0.5 mmol) in DMF (5 mL) was added HATU (209 mg, 0.55 mmol) and DIEA (194 mg, 1.5 mmol) at 0° C. The mixture was stirred for 15 min, then (S)-methyl pyrrolidine-3-carboxylate hydrochloride (83 mg, 0.5 mmol) was added. The reaction mixture was allowed to warm to room temperature and stirred for 16 h. The reaction was quenched by adding water and extracted with EtOAc (10 mL×3). The organic layer was combined, washed with brine and dried over Na₂SO₄. The solvent was evaporated under reduced pressure and the crude was purified by silica gel chromatography (18% MeOH/DCM) to afford 6-3 (98 mg, 38%) as a white solid.

### 6.4 The Synthesis of Compound I-147

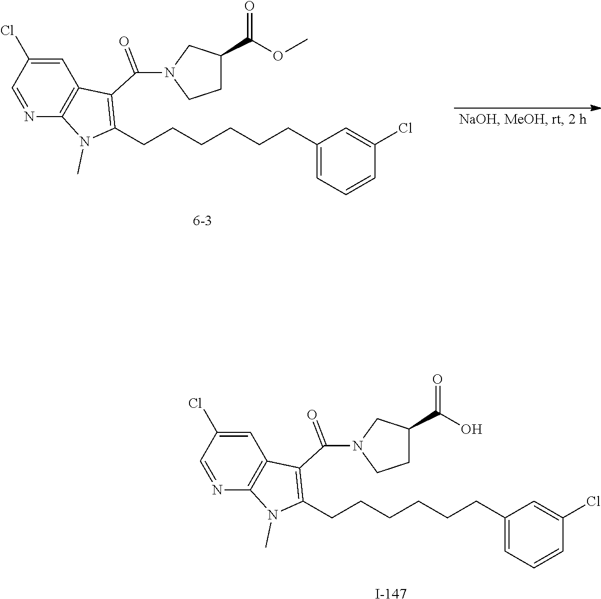 [SMILES: COC(=O)[C@H]1CCN(C(=O)c2c(CCCCCCc3cccc(Cl)c3)n(C)c3ncc(Cl)cc23)C1 ; "NaOH, MeOH, rt, 2 h" ; "6-3" ; Cn1c(CCCCCCc2cccc(Cl)c2)c(C(=O)N2CC[C@H](C(=O)O)C2)c2cc(Cl)cnc21 ; "I-147"]

To a mixture of 6-3 (98 mg, 0.19 mmol) in MeOH (5 mL) was added NaOH (2.0 M, 0.95 mL, 1.9 mmol) at 0° C., the reaction mixture was stirred at room temperature for 16 h. The mixture was acidified with HCl (1.0 M) to pH ˜3.0, extracted with EtOAc (10 mL×3). The combined organic layer was washed with brine, dried over anhydrous Na₂SO₄ and evaporated under reduced pressure. The crude was purified by Prep-HPLC to afford 1-147 (30 mg, 32%) as a white solid. ¹H NMR (400 MHz, DMSO-d₆) δ 12.47 (s, 1H), 8.25 (d, J=2.4 Hz, 1H), 7.92 (s, 1H), 7.30-7.20 (m, 3H), 7.14 (d, J=7.6 Hz, 1H), 3.77 (s, 3H), 3.59-3.42 (m, 4H), 3.07 (s, 1H), 2.90 (t, J=7.2 Hz, 2H), 2.56 (t, J=7.6 Hz, 2H), 2.10-2.04 (m, 2H), 1.61-1.51 (m, 4H), 1.31-1.30 (m, 4H). LC-MS m/z: 502.3 [M+H]⁺.

Additional exemplary compounds were prepared following methods substantially similar to the method described above and herein. Data for these compounds are provided in Tables 7 and 7a below.

TABLE 7
Characterization Data for Additional Exemplary Compounds

| Com- | | | |
|---|---|---|---|
| pound | | | |
| No. | Chemical Structure | M + 1 | ¹H NMR (400 MHz) |
| I-118 | 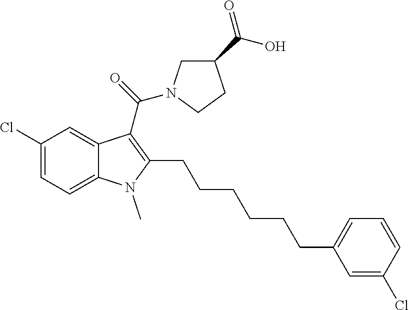 [SMILES: Cn1c(CCCCCCc2cccc(Cl)c2)c(C(=O)N2CC[C@H](C(=O)O)C2)c2cc(Cl)ccc21] | 500.9 | ¹H NMR (400 MHz, DMSO-d₆) δ 12.51 (br, 1H), 7.51 (d, J = 8.8 Hz, 1H), 7.38 (s, 1H), 7.30-7.21 (m, 3H), 7.16-7.13 (m, 2H), 3.71 (s, 3H), 3.60- 3.39 (m, 2H), 3.07 (br, 1H), 2.84 (t, J = 7.2 Hz, 2H), 2.54 (t, J = 4.0 Hz, 2H), 2.09-1.99 (m, 2H), 1.55-1.47 (m, 4H), 1.30-1.26 (m, 4H), 0.87-0.81 (m, 2H). |
| I-119 | 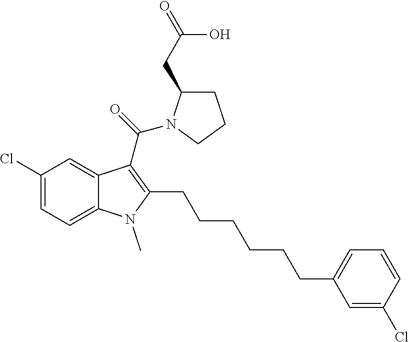 [SMILES: Cn1c(CCCCCCc2cccc(Cl)c2)c(C(=O)N2CCC[C@@H]2CC(=O)O)c2cc(Cl)ccc21] | 514.9 | ¹H NMR (400 MHz, DMSO-d₆) δ 12.28 (br, 1H), 7.50 (d, J = 8.8 Hz, 1H), 7.42 (s, 1H), 7.30-7.20 (m, 3H), 7.16-7.13 (m, 2H), 4.37 (br, 1H), 3.71 (s, 3H), 3.38-3.34 (m, 1H), 3.18-3.14 (m, 1H), 2.90-2.77 (m, 3H), 2.55 (t, J = 7.6 Hz, 3H), 2.15-2.11 (m, 1H), 1.83-1.74 (m, 2H), 1.60-1.51 (m, 5H), 1.30-1.26 (m, 4H). |
| I-120 | 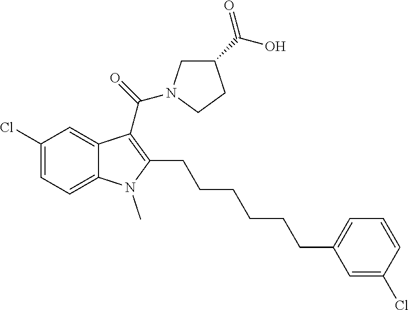 [SMILES: Cn1c(CCCCCCc2cccc(Cl)c2)c(C(=O)N2CC[C@@H](C(=O)O)C2)c2cc(Cl)ccc21] | 501.0 | ¹H NMR (400 MHz, DMSO-d₆) δ 12.53 (br, 1H), 7.51 (d, J = 8.8 Hz, 1H), 7.38 (s, 1H), 7.30-7.21 (m, 3H), 7.15 (dd, J = 8.8, 2.0 Hz, 2H), 3.71 (s, 3H), 3.55-3.39 (m, 2H), 3.07 (br, 1H), 2.84 (t, J = 7.2 Hz, 2H), 2.55 (t, J = 7.6 Hz, 2H), 2.09-2.01 (m, 2H), 1.55- 1.47 (m, 4H), 1.30-1.24 (m, 4H), 0.87-0.81 (m, 2H). |
| I-121 | 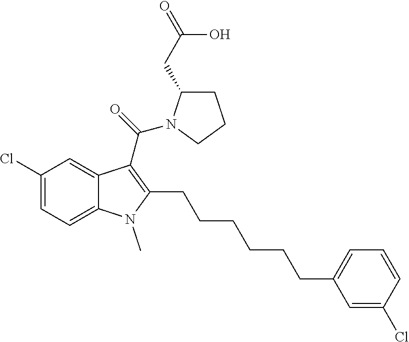 [SMILES: Cn1c(CCCCCCc2cccc(Cl)c2)c(C(=O)N2CCC[C@H]2CC(=O)O)c2cc(Cl)ccc21] | 514.9 | ¹H NMR (400 MHz, DMSO-d₆) δ 12.30 (br, 1H), 7.50 (d, J = 8.8 Hz, 1H), 7.42 (s, 1H), 7.30-7.20 (m, 3H), 7.16-7.13 (m, 2H), 4.34 (br, 1H), 3.71 (s, 3H), 3.39-3.36 (m, 1H), 3.16 (br, 1H), 2.91-2.76 (m, 3H), 2.57-2.53 (m, 3H), 2.15-2.08 (m, 1H), 1.83-1.72 (m, 2H), 1.60-1.51 (m, 5H), 1.29 (br, 4H). |
| I-148 | 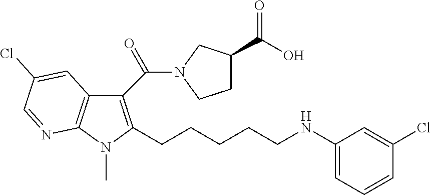 [SMILES: Cn1c(CCCCCNc2cccc(Cl)c2)c(C(=O)N2CC[C@H](C(=O)O)C2)c2cc(Cl)cnc21] | 503.1 | ¹H NMR (400 MHz, DMSO-d₆ + D₂O) δ 8.25 (d, J = 2.0 Hz, 1H), 7.92 (s, 1H), 7.04 (t, J = 8.0 Hz, 1H), 6.53 (t, J = 1.6 Hz, 1H), 6.49 (d, J = 2.0 Hz, 1H), 6.47 (d, J = 2.0 Hz, 1H), 3.78 (s, 3H), 3.57-3.40 (m, 4H), 3.07- 3.06 (m, 1H), 2.98-2.91 (m, 4H), 2.11-1.97 (m, 2H), 1.68-1.60 (m, 2H), 1.58-1.51 (m, 2H), 1.42-1.37 (m, 2H). |
| I-149 | 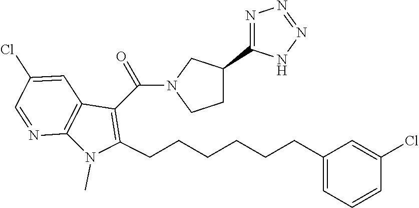 [SMILES: Cn1c(CCCCCCc2cccc(Cl)c2)c(C(=O)N2CC[C@H](c3nnn[nH]3)C2)c2cc(Cl)cnc21] | 526.2 | ¹H NMR (400 MHz, DMSO-d₆) δ 8.25 (d, J = 2.0 Hz, 1H), 7.96 (d, J = 2.4 Hz, 1H), 7.29-7.19 (m, 3H), 7.13 (d, J = 7.2 Hz, 1H), 3.93-3.72 (m, 5H), 3.66-3.46 (m, 2H), 2.89 (t, J = 7.6 Hz, 2H), 2.55 (t, J = 7.6 Hz, 4H), 2.42-2.34 (m, 1H), 2.26-2.01 (m, 1H), 1.61-1.49 (m, 4H), 1.34-1.24 (m, 4H). |
| I-151 | 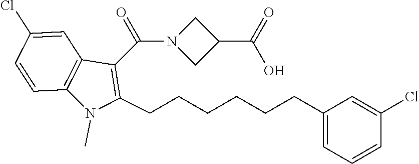 [SMILES: Cn1c(CCCCCCc2cccc(Cl)c2)c(C(=O)N2CC(C(=O)O)C2)c2cc(Cl)ccc21] | 487.1 | ¹H NMR (400 MHz, DMSO-d₆) δ 12.2 (br, 1H), 7.52 (d, J = 8.8 Hz, 1H), 7.47 (d, J = 2.0 Hz, 1H), 7.30- 7.26 (m, 2H), 7.23-7.21 (m, 1H), 7.19-7.14 (m, 2H), 4.12-4.10 (m, 2H), 4.00-3.96 (m, 2H), 3.72 (s, 3H), 3.44- 3.38 (m, 1H), 2.94 (t, J = 7.6 Hz, 2H), 2.56 (t, J = 7.6 Hz, 2H), 1.60-1.54 (m, 4H), 1.33-1.26 (m, 4H). |
| I-152 | 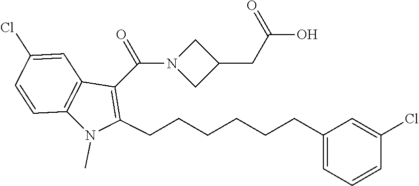 [SMILES: Cn1c(CCCCCCc2cccc(Cl)c2)c(C(=O)N2CC(CC(=O)O)C2)c2cc(Cl)ccc21] | 501.3 | ¹H NMR (400 MHz, DMSO-d₆) δ 12.2 (br, 1H), 7.51 (d, J = 8.8 Hz, 1H), 7.48 (d, J = 2.0 Hz, 1H), 7.30- 7.20 (m, 3H), 7.17-7.14 (m, 2H), 4.07-4.04 (m, 2H), 3.71 (s, 3H), 3.65- 3.58 (m, 2H), 2.94 (t, J = 7.6 Hz, 2H), 2.90-2.82 (m, 1H), 2.59-2.55 (m, 4H), 1.58-1.54 (m, 4H), 1.32-1.25 (m, 4H). |
| I-153 | 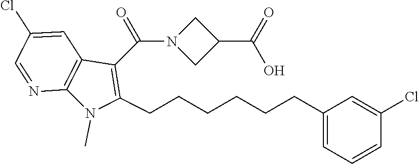 [SMILES: Cn1c(CCCCCCc2cccc(Cl)c2)c(C(=O)N2CC(C(=O)O)C2)c2cc(Cl)cnc21] | 488.1 | ¹H NMR (400 MHz, DMSO-d₆) δ 12.62 (s, 1H), 8.27 (d, J = 2.4 Hz, 1H), 7.94 (d, J = 2.0 Hz, 1H), 7.30- 7.24 (m, 2H), 7.22-7.20 (m, 1H), 7.15 (d, J = 7.6 Hz, 1H), 4.15 (t, J = 8.8 Hz, 2H), 4.00 (t, J = 6.4 Hz, 2H), 3.77 (s, 3H), 3.45-3.41 (m, 1H), 2.99 (t, J = 7.2 Hz, 2H), 2.57 (t, J = 7.6 Hz, 2H), 1.63-1.53 (m, 4H), 1.39-1.29 (m, 4H). |
| I-154 | 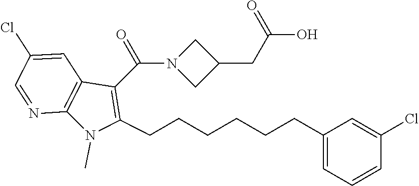 [SMILES: Cn1c(CCCCCCc2cccc(Cl)c2)c(C(=O)N2CC(CC(=O)O)C2)c2cc(Cl)cnc21] | 502.1 | ¹H NMR (400 MHz, DMSO-d₆) δ 12.23 (s, 1H), 8.26 (d, J = 2.0 Hz, 1H), 7.98 (d, J = 2.0 Hz, 1H), 7.30- 7.25 (m, 2H), 7.22-7.20 (m, 1H), 7.15 (d, J = 7.6 Hz, 1H), 4.08-4.06 (m, 2H), 3.77 (s, 3H), 3.66-3.64 (m, 2H), 2.99 (t, J = 7.6 Hz, 2H), 2.90-2.83 (m, 1H), 2.61-2.55 (m, 4H), 1.61-1.53 (m, 4H), 1.38-1.27 (m, 4H). |
| I-155 | 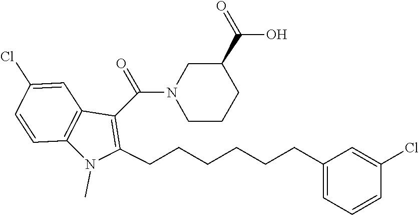 [SMILES: Cn1c(CCCCCCc2cccc(Cl)c2)c(C(=O)N2CCC[C@H](C(=O)O)C2)c2cc(Cl)ccc21] | 514.9 | ¹H NMR (400 MHz, DMSO-d₆) δ 12.35 (s, 1H), 7.51 (d, J = 8.8 Hz, 1H), 7.41-7.35 (m, 1H), 7.30-7.20 (m, 3H), 7.17-7.13 (m, 2H), 4.20-4.00 (m, 1H), 3.95-3.78 (m, 1H), 3.72 (s, 3H), 3.24 (t, J = 11.2 Hz, 1H), 3.05-2.80 (m, 3H), 2.56 (t, J = 7.6 Hz, 2H), 2.33-2.29 (m, 1H), 1.99-1.93 (m, 1H), 1.67-1.55 (m, 6H), 1.31-1.23 (m, 5H). |
| I-156 | 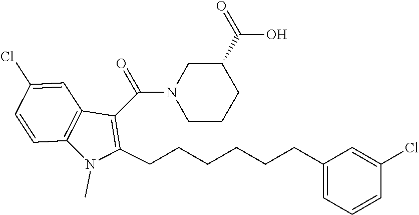 [SMILES: Cn1c(CCCCCCc2cccc(Cl)c2)c(C(=O)N2CCC[C@@H](C(=O)O)C2)c2cc(Cl)ccc21] | 514.8 | ¹H NMR (400 MHz, DMSO-d₆) δ 12.38 (s, 1H), 7.51 (d, J = 8.4 Hz, 1H), 7.41-7.35 (m, 1H), 7.30-7.20 (m, 3H), 7.17-7.13 (m, 2H), 4.09-4.07 (m, 1H), 3.83-3.80 (m, 1H), 3.72 (s, 3H), 3.24 (t, J = 11.6 Hz, 1H), 3.05-2.80 (m, 3H), 2.56 (t, J = 7.6 Hz, 2H), 2.33-2.29 (m, 1H), 2.01-1.93 (m, 1H), 1.67-1.46 (m, 6H), 1.31-1.23 (m, 5H). |
| I-157 | 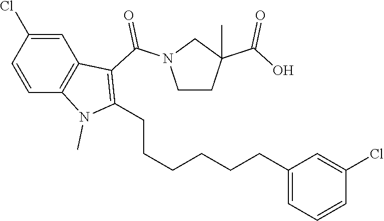 [SMILES: Cn1c(CCCCCCc2cccc(Cl)c2)c(C(=O)N2CCC(C)(C(=O)O)C2)c2cc(Cl)ccc21] | 515.2 | ¹H NMR (400 MHz, DMSO-d₆) δ 12.60 (s, 1H), 7.51 (d, J = 8.8 Hz, 1H), 7.36 (s, 1H), 7.30-7.21 (m, 3H), 7.15 (dd, J = 8.4, 2.0 Hz, 2H), 3.71 (s, 3H), 3.65-3.40 (m, 2H), 3.28-2.90 (m, 2H), 2.83 (s, 2H), 2.56 (t, J = 8.0 Hz, 2H), 2.30-2.20 (m, 1H), 1.87-1.74 (m, 1H), 1.58-1.50 (m, 4H), 1.31-1.12 (m, 7H). |
| I-303 | 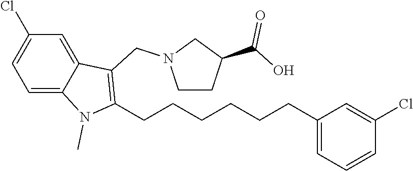 [SMILES: Cn1c(CCCCCCc2cccc(Cl)c2)c(CN2CC[C@H](C(=O)O)C2)c2cc(Cl)ccc21] | 487.3 | ¹H NMR (400 MHz, CDCl₃) δ 7.37 (d, J = 2.0 Hz, 1H), 7.21-7.12 (m, 5H), 7.02 (d, J = 7.2 Hz, 1H), 3.95- 3.20 (m, 9H), 2.95-2.80 (m. 2H), 2.58-2.30 (m, 3H), 2.10-1.98 (m, 1H), 1.75-1.50 (m, 5H), 1.45-1.30 (m, 4H). |
| I-174 | 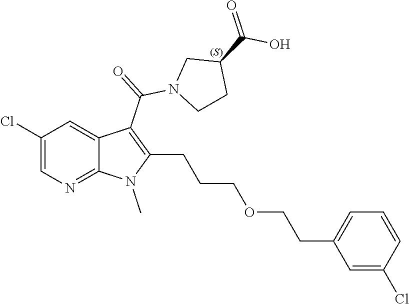 | 504.2 | ¹H NMR (400 MHz, DMSO-d₆) δ 12.44 (s, 1H), 8.25 (d, J = 2.4 Hz, 1H), 7.92 (s, 1H), 7.36-7.27 (m, 2H), 7.26-7.24 (m, 1H), 7.23-7.18 (m, 1H), 3.73 (s, 3H), 3.57 (t, J = 6.8 Hz, 4H), 3.40 (t, J = 6.0 Hz, 4H), 3.06 (s, 1H), 2.91 (t, J = 7.2 Hz, 2H), 2.78 (t, J = 6.8 Hz, 2H), 2.22-1.98 (m, 2H), 1.85- 1.75 (m, 2H). |
| I-175 | 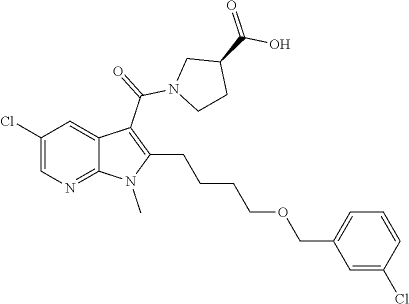 [SMILES: Cn1c(CCCCOCc2cccc(Cl)c2)c(C(=O)N2CC[C@H](C(=O)O)C2)c2cc(Cl)cnc21] | 503.9 | ¹H NMR (400 MHz, DMSO-d₆) δ 12.24 (br, 1H), 8.25 (d, J = 2.0 Hz, 1H), 7.93 (s, 1H), 7.39-7.31 (m, 3H), 7.27 (t, J = 3.6 Hz, 1H), 4.45 (s, 2H), 3.78 (s, 3H), 3.27-3.60 (m, 6H), 3.08 (br, 1H), 2.95 (t, J = 7.2 Hz, 2H), 2.09 (br, 2H), 1.72-1.65 (m, 2H), 1.62- 1.55 (m, 2H). |
| I-178 | 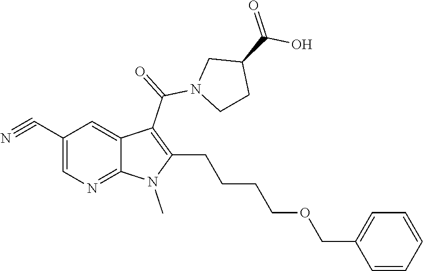 [SMILES: Cn1c(CCCCOCc2ccccc2)c(C(=O)N2CC[C@H](C(=O)O)C2)c2cc(C#N)cnc21] | 461.0 | ¹H NMR (400 MHz, DMSO-d₆) δ 8.65 (d, J = 1.6 Hz, 1H), 8.38 (s, 1H), 7.22-7.36 (m, 5H), 4.43 (s, 2H), 3.81 (s, 3H), 3.32-3.55 (m, 7H), 2.95 (t, J = 7.6 Hz, 2H), 1.90-2.20 (m, 2H), 1.63-1.70 (m, 2H), 1.52-1.61 (m, 2H). |
| I-179 | 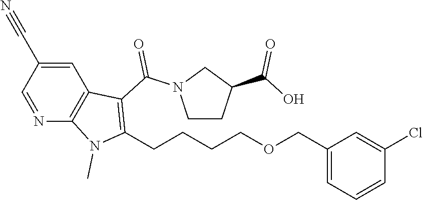 [SMILES: Cn1c(CCCCOCc2cccc(Cl)c2)c(C(=O)N2CC[C@H](C(=O)O)C2)c2cc(C#N)cnc21] | 495.3 | ¹H NMR (400 MHz, DMSO-d₆) δ 12.52 (br., 1H), 8.65 (d, J = 2.0 Hz, 1H), 8.39 (s, 1H), 7.31-7.39 (m, 3H), 7.24-7.30 (m, 1H), 4.45 (s, 2H), 3.82 (s, 3H), 3.30-3.60 (m, 7H), 2.96 (t, J = 7.6 Hz, 2H), 1.90-2.20 (m, 2H), 1.65-1.72 (m, 2H), 1.54-1.62 (m, 2H). |
| I-181 | 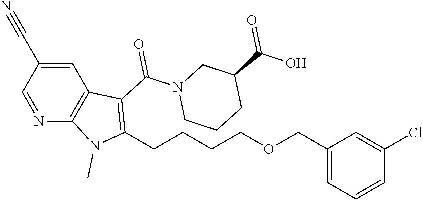 [SMILES: Cn1c(CCCCOCc2cccc(Cl)c2)c(C(=O)N2CCC[C@H](C(=O)O)C2)c2cc(C#N)cnc21] | 509.2 | ¹H NMR (400 MHz, DMSO-d₆) δ 12.38 (br., 1H), 8.83 (d, J = 2.0 Hz, 1H), 8.35-8.40 (m, 1H), 7.26-7.38 (m, 4H), 4.45 (s, 2H), 3.82 (s, 3H), 3.55- 3.78 (m, 2H), 3.46 (t, J = 6.4 Hz, 2H), 2.55-3.35 (m, 5H), 1.89-2.01 (m, 1H), 1.25-1.78 (m, 7H). |
| I-183 | 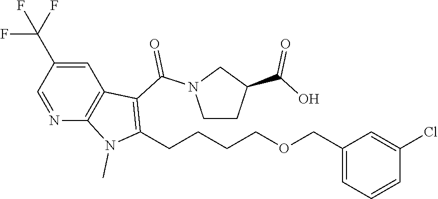 [SMILES: Cn1c(CCCCOCc2cccc(Cl)c2)c(C(=O)N2CC[C@H](C(=O)O)C2)c2cc(C(F)(F)F)cnc21] | 538.2 | ¹H NMR (400 MHz, DMSO-d₆) δ12.50 (br, 1H), 8.61 (s, 1H), 8.18 (s, 1H), 7.38-7.19 (m, 4H), 4.45 (s, 2H), 3.84 (s, 3H), 3.53-3.44 (m, 6H), 3.07- 2.96 (m, 3H), 2.08 (br, 2H), 1.72-1.66 (m, 2H), 1.61-1.57 (m, 2H). |
| I-184 | 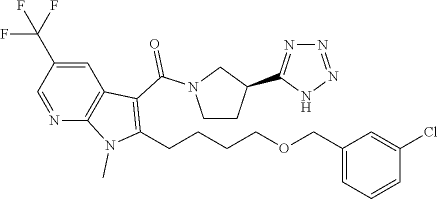 [SMILES: Cn1c(CCCCOCc2cccc(Cl)c2)c(C(=O)N2CC[C@H](c3nnn[nH]3)C2)c2cc(C(F)(F)F)cnc21] | 562.2 | ¹H NMR (400 MHz, DMSO-d₆) δ 8.61 (d, J = 1.2 Hz, 1H), 8.23 (s, 1H), 7.37-7.30 (m, 3H), 7.26 (d, J = 6.8 Hz, 1H), 4.45 (s, 2H), 3.84 (s, 3H), 3.77-3.44 (m, 7H), 2.97 (t, J = 7.2 Hz, 2H), 2.33 (br, 1H), 2.14 (br, 1H), 1.70-1.65 (m, 2H), 1.61-1.57 (m, 2H). |
| I-187 | 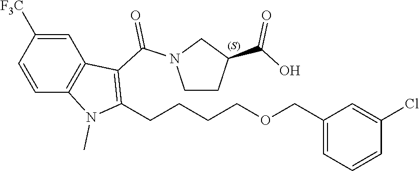 | 537.3 | ¹H NMR (400 MHz, DMSO-d₆) δ 12.50 (br., 1H), 7.70-7.68 (m, 2H), 7.45 (dd, J = 8.8, 1.2 Hz, 1H), 7.39- 7.32 (m, 3H), 7.27-7.26 (m, 1H), 4.45 (s, 2H), 3.78 (s, 3H), 3.60-3.43 (m, 6H), 3.10-3.01 (m, 1H), 2.92 (t, J = 6.4 Hz, 2H), 2.14-2.00 (m, 2H), 1.68- 1.62 (m, 2H), 1.60-1.55 (m, 2H). |
| I-188 | 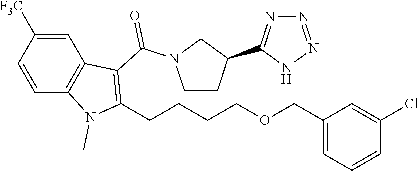 [SMILES: Cn1c(CCCCOCc2cccc(Cl)c2)c(C(=O)N2CC[C@H](c3nnn[nH]3)C2)c2cc(C(F)(F)F)ccc21] | 561.3 | ¹H NMR (400 MHz, DMSO-d₆) δ 7.74 (s, 1H), 7.70 (d, J = 8.8 Hz, 1H), 7.45 (dd, J = 8.8, 1.2 Hz, 1H), 7.37- 7.31 (m, 3H), 7.27-7.25 (m, 1H), 4.44 (s, 2H), 3.90-3.73 (m, 5H), 3.44 (t, J = 6.0 Hz, 2H), 3.38-3.26 (m, 3H), 2.91 (t, J = 7.6 Hz, 2H), 2.42-2.28 (m, 1H), 2.20-2.08 (m, 1H), 1.67-1.54 (m, 4H). |
| I-223 | 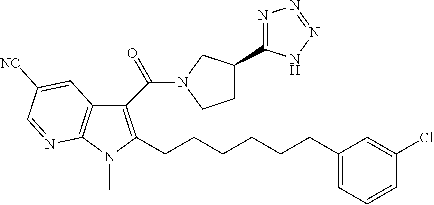 [SMILES: Cn1c(CCCCCCc2cccc(Cl)c2)c(C(=O)N2CC[C@H](c3nnn[nH]3)C2)c2cc(C#N)cnc21] | 517.3 | ¹H NMR (400 MHz, MeOD-d₄) δ 8.56 (d, J = 1.6 Hz, 1H), 8.30 (d, J = 1.6 Hz, 1H), 7.21-7.07 (s, 4H), 3.87 (m, 7H), 3.54 (br, 1H), 2.98 (t, J = 7.2 Hz, 2H), 2.59 (t, J = 7.2 Hz, 2H), 2.37 (br, 2H), 1.67-1.57 (m, 4H), 1.44-1.29 (m, 4H). |
| I-224 | 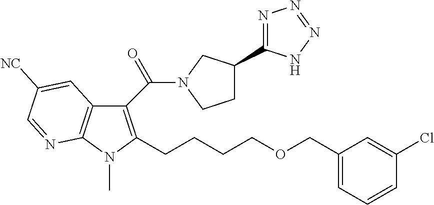 [SMILES: Cn1c(CCCCOCc2cccc(Cl)c2)c(C(=O)N2CC[C@H](c3nnn[nH]3)C2)c2cc(C#N)cnc21] | 519.3 | ¹H NMR (400 MHz, DMSO-d₆) δ 8.66 (d, J = 1.6 Hz, 1H), 8.43 (d, J = 1.2 Hz, 1H), 7.37-7.25 (m, 4H), 4.45 (s, 2H), 3.90-3.25 (m, 10H), 2.97 (t, J = 6.8 Hz, 2H), 2.40-2.20 (m, 2H), 1.69-1.56 (m, 4H). |
| I-225 | 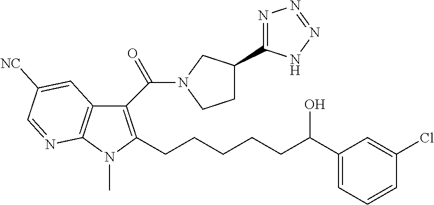 [SMILES: Cn1c(CCCCCC(O)c2cccc(Cl)c2)c(C(=O)N2CC[C@H](c3nnn[nH]3)C2)c2cc(C#N)cnc21] | 533.3 | ¹H NMR (400 MHz, DMSO-d₆) δ 8.63 (s, 1H), 8.45 (s, 1H), 7.35-7.22 (m, 4H), 4.52 (t, J = 6.0 Hz, 1H), 3.81 (s, 3H), 3.64-3.43 (m, 6H), 2.91 (s, 2H), 2.34-1.99 (m, 2H), 1.56 (br., 4H), 1.29 (br., 4H). |
| I-237 | 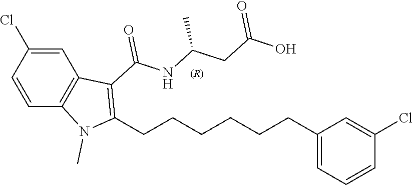 | 489.1 | ¹H NMR (400 MHz, DMSO-d₆) δ 12.25 (br, 1H), 7.69 (d, J = 1.6 Hz, 1H), 7.63 (d, J = 8.0 Hz, 1H), 7.49 (d, J = 8.8 Hz, 1H), 7.30-7.18 (m, 3H), 7.17-7.14 (m, 2H), 4.40-4.33 (m, 1H), 3.70 (s, 3H), 3.08-2.98 (m, 2H), 2.63- 2.54 (m, 3H), 2.44 (dd, J = 15.6, 6.8 Hz, 1H), 1.59-1.55 (m, 4H), 1.39- 1.30 (m, 4H), 1.20 (d, J = 6.4 Hz, 3H). |
| I-238 | 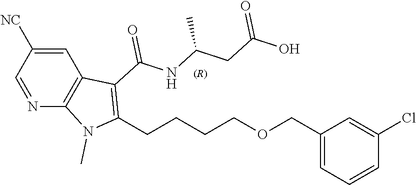 | 483.3 | ¹H NMR (400 MHz, DMSO-d₆) δ 12.28 (s, 1H), 8.67 (d, J = 2.0 Hz, 1H), 8.47 (d, J = 2.0 Hz, 1H), 7.88 (d, J = 8.4 Hz, 1H), 7.39-7.26 (m, 4H), 4.45 (s, 2H), 4.40-4.33 (m, 1H), 3.82 (s, 3H), 3.47 (t, J = 6.0 Hz, 2H), 3.21- 3.10 (m, 2H), 2.65-2.59 (m, 1H), 2.50-2.45 (m, 1H), 1.70-1.65 (m, 4H), 1.21 (d, J = 6.8 Hz, 3H). |
| I-239 | 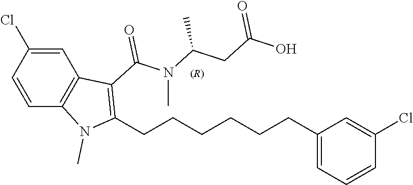 | 503.2 | ¹H NMR (400 MHz, DMSO-d₆) δ 12.26 (s, 1H), 7.49 (d, J = 7.6 Hz, 1H), 7.35 (br., 1H), 7.30-7.20 (m, 3H), 7.14 (d, J = 7.6 Hz, 2H), 4.92 (br., 1H), 3.70 (s, 3H), 2.76 (br., 7H); 2.62-2.57 (m, 2H), 1.55-1.52 (m, 4H), 1.47-1.05 (m, 7H). |
| I-240 | 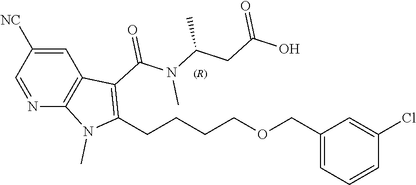 | 497.2 | ¹H NMR (400 MHz, DMSO-d₆) δ 8.62 (d, J = 1.6 Hz, 1H), 8.37 (s, 1H), 7.37-7.24 (m, 4H), 4.43 (s, 2H), 3.81 (s, 3H), 3.43 (t, J = 5.6 Hz, 2H), 2.90- 2.73 (m, 5H), 2.49-2.47 (m, 1H), 2.33-2.24 (m, 2H), 1.67-1.52 (m, 4H), 1.11 (s, 3H). |
| I-241 | 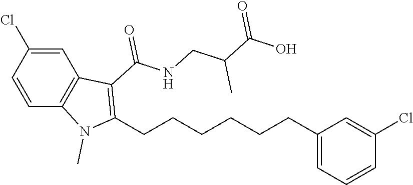 [SMILES: CC(CNC(=O)c1c(CCCCCCc2cccc(Cl)c2)n(C)c2ccc(Cl)cc12)C(=O)O] | 489.3 | ¹H NMR (400 MHz, DMSO-d₆) δ 12.31 (s, 1H), 7.72-7.70 (m, 2H), 7.50 (d, J = 8.8 Hz, 1H), 7.31-7.14 (m, 5H), 3.71 (s, 3H), 3.51-3.43 (m, 1H), 3.29-3.27 (m, 1H), 3.04 (t, J = 7.6 Hz, 2H), 2.74-2.67 (m, 1H), 2.56 (d, J = 7.6 Hz, 2H), 1.60-1.50 (m, 4H), 1.40-1.30 (m, 4H), 1.11 (d, J = 7.2 Hz, 3H). |
| I-242 | 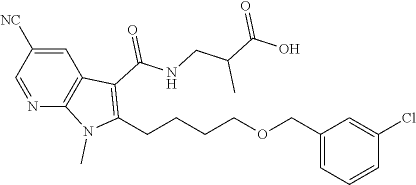 [SMILES: CC(CNC(=O)c1c(CCCCOCc2cccc(Cl)c2)n(C)c2ncc(C#N)cc12)C(=O)O] | 483.3 | ¹H NMR (400 MHz, DMSO-d₆) δ 12.41 (s, 1H), 8.67 (d, J = 1.2 Hz, 1H), 8.50 (d, J = 1.2 Hz, 1H), 8.02 (t, J = 5.2 Hz, 1H), 7.39-7.26 (m, 4H), 4.45 (s, 2H), 3.82 (s, 3H), 3.49-3.43 (m, 2H), 3.39-3.32 (m, 2H), 3.17 (t, J = 6.4 Hz, 2H), 2.79-2.70 (m, 1H), 1.66 (s, 4H), 1.11 (d, J = 7.2 Hz, 3H). |
| I-287 | 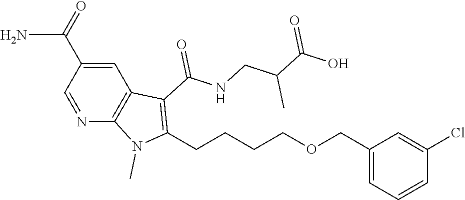 [SMILES: CC(CNC(=O)c1c(CCCCOCc2cccc(Cl)c2)n(C)c2ncc(C(N)=O)cc12)C(=O)O] | 501.2 | ¹H NMR (400 MHz, DMSO-d₆) δ 12.30 (s, 1H), 8.75 (d, J = 2.0 Hz, 1H), 8.57 (d, J = 1.2 Hz, 1H), 8.00 (s, 1H), 7.83 (t, J = 5.2 Hz, 1H), 7.42- 7.32 (m, 4H), 7.28-7.27 (m, 1H), 4.46 (s, 2H), 3.80 (s, 3H), 3.54-3.46 (m, 3H), 3.38-3.35 (m, 1H), 3.15 (t, J = 6.8 Hz, 2H), 2.76-2.67 (m, 1H), 1.65 (br., 4H), 1.12 (d, J = 7.2 Hz, 3H). |
| I-243 | 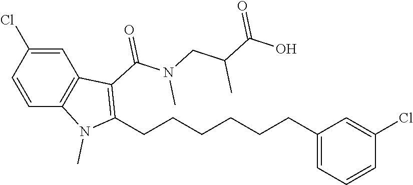 [SMILES: CC(CN(C)C(=O)c1c(CCCCCCc2cccc(Cl)c2)n(C)c2ccc(Cl)cc12)C(=O)O] | 502.9 | ¹H NMR (400 MHz, DMSO-d₆) δ 12.50 (br., 1H), 7.48 (d, J = 8.8 Hz, 1H), 7.36 (s, 1H), 7.29-7.19 (m, 3H), 7.16-7.11 (m, 2H), 3.70 (s, 3H), 3.50- 3.20 (m, 2H), 2.91 (s, 3H), 2.80 (br., 3H), 2.55-2.50 (m, 2H), 1.54-1.49 (m, 4H), 1.38-1.22 (m, 4H), 1.05 (br., 3H). |
| I-244 | 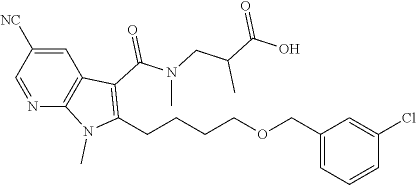 [SMILES: CC(CN(C)C(=O)c1c(CCCCOCc2cccc(Cl)c2)n(C)c2ncc(C#N)cc12)C(=O)O] | 497.2 | ¹H NMR (400 MHz, DMSO-d₆) δ 8.66 (d, J = 1.6 Hz, 1H), 8.27 (s, 1H), 7.39-7.32 (m, 3H), 7.27 (d, J = 6.8 Hz, 1H), 4.45 (s, 2H), 3.82 (s, 3H), 3.59 (br., 2H), 3.46 (t, J = 4.4 Hz, 2H), 2.91 (s, 6H), 1.69-1.62 (m, 4H), 1.07 (br., 3H). |
| I-289 | 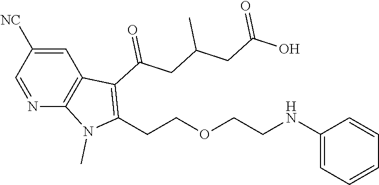 [SMILES: CC(CC(=O)O)CC(=O)c1c(CCOCCNc2ccccc2)n(C)c2ncc(C#N)cc12] | 449.3 | ¹H NMR (400 MHz, DMSO-d₆) δ 8.86 (d, J = 1.6 Hz, 1H), 8.73 (d, J = 1.6 Hz, 1H), 7.01 (t, J = 8.0 Hz, 2H), 6.51-6.47 (m, 3H), 5.40 (br, 1H), 3.88 (s, 3 H), 3.76 (t, J = 6.4 Hz, 2H), 3.54- 3.47 (m, 4H), 3.16-3.09 (m, 3H), 2.89 (dd, J = 16.4, 8.0 Hz, 1H), 2.50-2.46 (m, 1H), 2.36 (dd, J = 15.2, 6.0Hz, 1H), 2.15 (dd, J = 15.6, 8.0 Hz, 1H), 0.95 (d, J = 6.8 Hz, 3H). |
| I-338 | 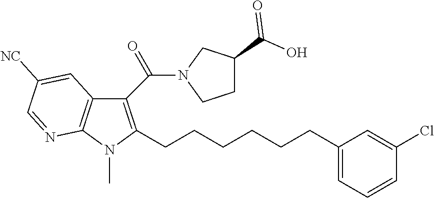 [SMILES: Cn1c(CCCCCCc2cccc(Cl)c2)c(C(=O)N2CC[C@H](C(=O)O)C2)c2cc(C#N)cnc21] | 493.00 | ¹H NMR (400 MHz, DMSO-d₆) δ 12.60 (br., 1H), 8.65 (d, J = 2.0 Hz, 1H), 8.41-8.38 (m, 1H), 7.42-7.13 (m, 4H), 3.82 (s, 3H), 3.79-3.40 (m, 4H), 3.154-2.87 (m, 3H), 2.56 (t, J = 7.6 Hz, 2H), 2.25-1.90 (m, 2H), 1.80- 1.50 (m, 4H), 1.49-1.38 (m, 1H), 1.36-1.23 (m, 3H). |
| I-339 | 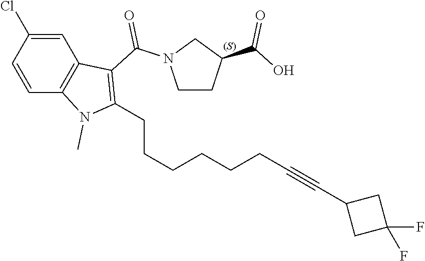 | 505.3 | ¹H NMR (400 MHz, DMSO-d₆) δ 12.52 (br., 1H), 7.51 (d, J = 8.4 Hz, 1H), 7.39 (s, 1H), 7.15 (dd, J = 8.8 Hz, 2.0 Hz, 1H), 3.72 (s, 3H), 3.65- 3.43 (m, 4H), 3.07 (s, 1H), 2.94-2.83 (m, 5H), 2.55-2.53 (m, 1H), 2.50-2.48 (m, 1H), 2.16-2.04 (m, 4H), 1.57-1.54 (m, 2H), 1.42-1.28 (m, 6H). |
| I-340 | 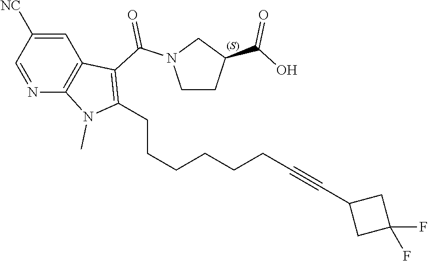 | 497.3 | ¹H NMR (400 MHz, DMSO-d₆) δ 12.62 (s, 1H), 8.66 (d, J = 1.6 Hz, 1H), 8.39 (s, 1H), 3.83 (s, 3H), 3.70- 3.41 (m, 4H), 3.08 (br., 1H), 2.93- 2.87 (m, 5H), 2.51-2.45 (m, 2H), 2.16-2.13 (m, 4H), 1.64-1.57 (m, 2H), 1.41-1.32 (m, 6H). |
| I-341 | 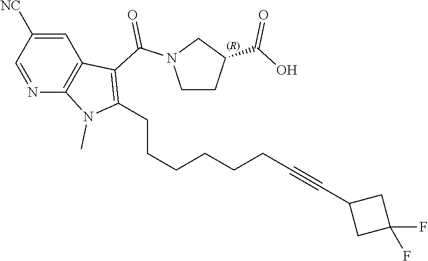 | 497.3 | ¹H NMR (400 MHz, DMSO-d₆) δ 12.56 (s, 1H), 8.65 (d, J = 2.0 Hz, 1H), 8.39 (s, 1H), 3.83 (s, 3H), 3.70- 3.40 (m, 4H), 3.08 (s, 1H), 2.93-2.87 (m, 5H), 2.55-2.51 (m, 2H), 2.16-2.13 (m, 4H), 1.62-1.58 (m, 2H), 1.42-1.30 (m, 6H). |
| I-342 | 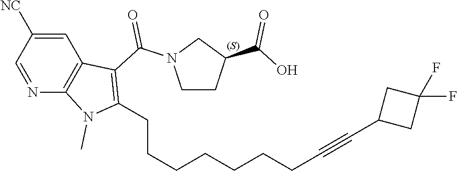 | 511.2 | ¹H NMR (400 MHz, DMSO-d6) δ 12.40 (br., 1H), 8.65 (d, J = 1.6 Hz, 1H), 8.38 (s, 1H), 3.83 (s, 3H), 3.71- 3.35 (m, 4H), 3.16-3.00 (m, 1H), 2.97-2.85 (m, 5H), 2.48-2.40 (m, 2H), 2.20-1.93 (m, 4H), 1.65-1.54 (m, 2H), 1.45-1.35 (m, 2H), 1.34-1.25 (m, 6H). |
| I-343 | 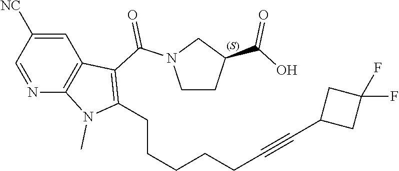 | 483.3 | ¹H NMR (400 MHz, DMSO-d6) δ 12.61 (br., 1H), 8.65 (d, J = 2.0 Hz, 1H), 8.39 (s, 1H), 3.83 (s, 3H), 3.75- 3.38 (m, 4H), 3.08 (br., 1H), 2.96- 2.80 (m, 5H), 2.49-2.38 (m, 2H), 2.22-1.93 (m, 4H), 1.67-1.55 (m, 2H), 1.48-1.28 (m, 4H). |
| I-344 | 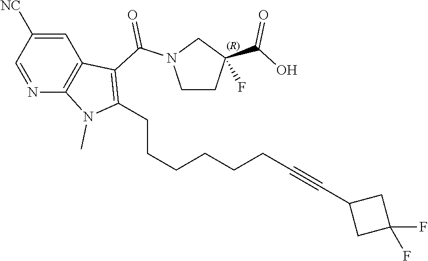 | 515.2 | ¹H NMR (400 MHz, DMSO-d6) δ 13.85 (br., 1H), 8.66 (d, J = 2.0 Hz, 1H), 8.44 (d, J = 1.6 Hz, 1H), 4.01- 3.77 (m, 5H), 3.76-3.56 (m, 2H), 2.96-2.84 (m, 5H), 2.58-2.52 (m, 2H), 2.49-2.21 (m, 2H), 2.15 (t, J = 6.4 Hz, 2H), 1.68-1.51 (m, 2H), 1.44-1.30 (m, 6H). |
| I-345 | 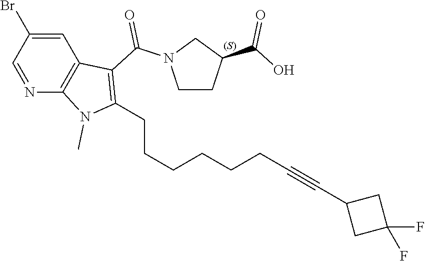 | 550.2 | ¹H NMR (400 MHz, DMSO-d6) δ 12.53 (br., 1H), 8.31 (d, J = 2.0 Hz, 1H), 8.03 (s, 1H), 3.77 (s, 3H), 3.72- 3.38 (m, 4H), 3.08 (s, 1H), 2.95-2.84 (m, 5H), 2.55-2.53 (m, 2H), 2.16-1.99 (m, 4H), 1.63-1.56 (m, 2H), 1.42-1.24 (m, 6H). |
| I-346 | 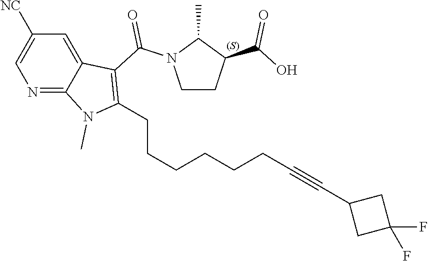 | 511.2 | ¹H NMR (400 MHz, DMSO-d6) δ 12.71 (br., 1H), 8.65 (d, J = 2.0 Hz, 1H), 8.33 (s, 1H), 4.30 (s, 1H), 3.82 (s, 3H), 3.61-3.40 (m, 2H), 3.03-2.83 (m, 5H), 2.79-2.67 (m, 1H), 2.56-2.51 (m, 2H), 2.21-2.02 (m, 3H), 1.85 (br., 1H), 1.66-1.50 (m, 2H), 1.46-0.93 (m, 9H). |
| I-347 | 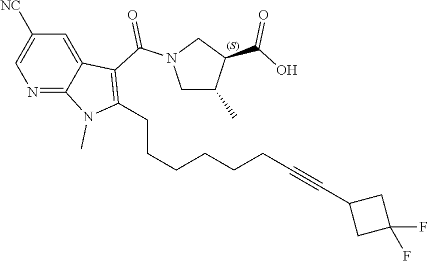 | 511.3 | ¹H NMR (400 MHz, DMSO-d6) δ 12.62 (br., 1H), 8.65 (d, J = 2.0 Hz, 1H), 8.41 (s, 1H), 3.83 (s, 3H), 3.77- 3.34 (m, 4H), 3.08 (br., 1H), 2.98- 2.86 (m, 5H), 2.80-2.61 (m, 1H), 2.60-2.53 (m, 1H), 2.50-2.23 (m, 1H), 2.14 (t, J = 6.0Hz, 2H), 1.66-1.53 (m, 2H), 1.43-1.29 (m, 6H), 1.20-0.95 (m, 3H). |
| I-348 | 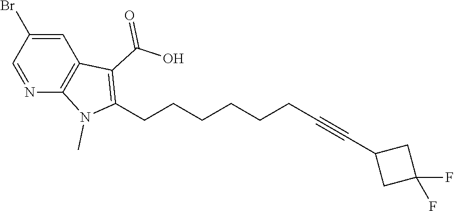 [SMILES: Cn1c(CCCCCCC#CC2CC(F)(F)C2)c(C(=O)O)c2cc(Br)cnc21] | 455.1 | ¹H NMR (400 MHz, DMSO-d6) δ 12.42 (s, 1H), 8.36 (s, 2H), 3.80 (s, 3H), 3.20 (t, J = 8.0 Hz, 2H), 2.94- 2.90 (m, 3H), 2.55-2.51 (m, 2H), 2.16-2.14 (m, 2H), 1.60 (s, 2H), 1.43- 1.39 (m, 6H). |
| I-349 | 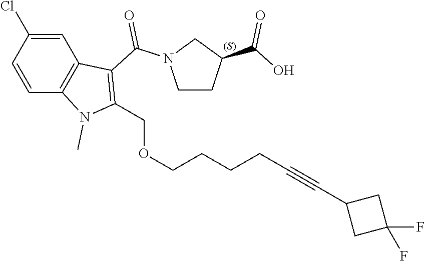 | 507.2 | ¹H NMR (400 MHz, DMSO-d6) δ 12.52 (s, 1H), 7.57 (d, J = 8.8 Hz, 1H), 7.49 (s, 1H), 7.23 (dd, J = 8.8 Hz, 2.0 Hz, 1H), 4.66 (s, 2H), 3.78 (s, 3H), 3.75-3.46 (m, 2H), 3.43 (t, J = 6.4 Hz, 2H), 3.41-3.35 (m, 2H), 3.09 (br., 1H), 2.92-2.88 (m, 3H), 2.52- 2.47 (m, 2H), 2.14 (t, J = 6.4 Hz, 2H), 2.12-1.92 (m, 2H), 1.61-1.54 (m, 2H), 1.48-1.40 (m, 2H). |
| I-350 | 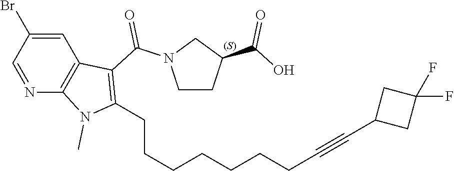 | 564.1 | ¹H NMR (400 MHz, DMSO-d6) δ 12.47 (s, 1H), 8.31 (d, J = 2.0 Hz, 1H), 8.04 (s, 1H), 3.77 (s, 3H), 3.68- 3.35 (m, 4H), 3.16-3.00 (m, 1H), 2.95-2.84 (m, 5H), 2.56-2.51 (m, 1H), 2.49-2.43 (m, 1H), 2.19-1.96 (m, 4H), 1.65-1.53 (m, 2H), 1.45-1.35 (m, 2H), 1.34-1.21 (m, 6H). |
| I-351 | 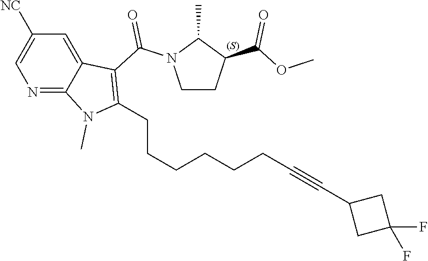 | 525.3 | ¹H NMR (400 MHz, DMSO-d6) δ 8.66 (d, J = 1.6 Hz, 1H), 8.32 (s, 1H), 4.31 (br., 1H), 3.83 (s, 3H), 3.68 (s, 3H), 3.58-3.46 (m, 1H), 3.28-3.13 (m, 1H), 3.06-2.96 (m, 1H), 2.95-2.80 (m, 5H), 2.57-2.52 (m, 2H), 2.19-2.04 (m, 3H), 2.00-1.76 (m, 1H), 1.69-1.50 (m, 2H), 1.45-1.17 (m, 9H). |

TABLE 7a
Additional Characterization Data for Additional Exemplary Compounds

| Compound | Rt (Min) | Rt (Min) |
|---|---|---|
| No. | (LCMS) | (HPLC) |
| I-338 | 1.928 | 9.561 |
| I-339 | 1.942 | 9.172 |
| I-340 | 1.825 | 8.954 |
| I-341 | 1.827 | 8.982 |
| I-342 | 1.887 | 9.260 |
| I-343 | 1.759 | 8.522 |
| I-344 | 1.851 | 9.117 |
| I-345 | 1.919 | 9.572 |
| I-346 | 1.891 | 9.386 |
| I-347 | 1.872 | 9.272 |
| I-348 | 2.116 | 10.832 |
| I-349 | 1.874 | 9.291 |
| I-350 | 1.985 | 9.983 |
| I-351 | 2.031 | 10.281 |

### Example 7: Synthesis of compound 1-13

### Synthetic Scheme for compound I-13

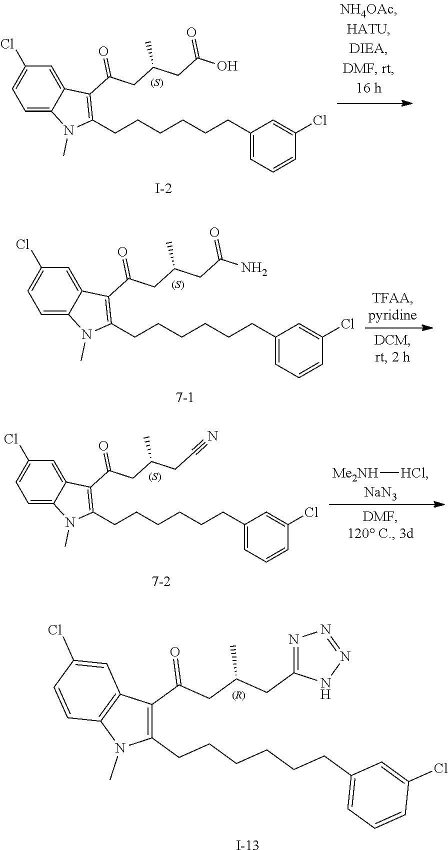

### 7.1 The Synthesis of Intermediate 7-1

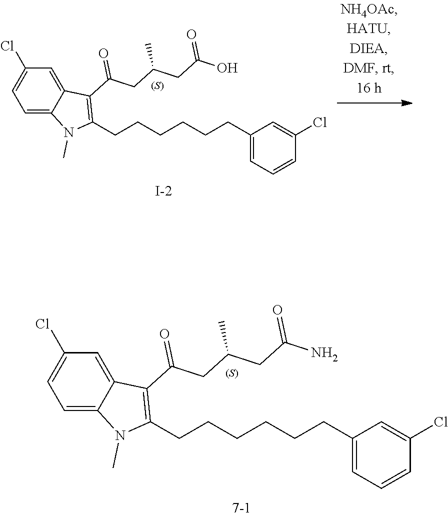

To a mixture of I-2 (250 mg, 0.51 mmol), ammonium acetate (60 mg, 0.77 mmol), HATU (195 mg, 0.51 mmol) and DIEA (199 mg, 1.54 mmol) in DMF (5 mL), the reaction mixture was stirred at room temperature overnight. The reaction was quenched by adding water and extracted with EtOAc. The organic layer was combined, washed with brine and dried over anhydrous Na₂SO₄. The solvent was evaporated under reduced pressure and the crude was purified by silica gel chromatography (20% EtOAc/Hex) to afford 7-1 (160 mg, 64%) as a yellow solid.

### 7.2 The Synthesis of Intermediate 7-2

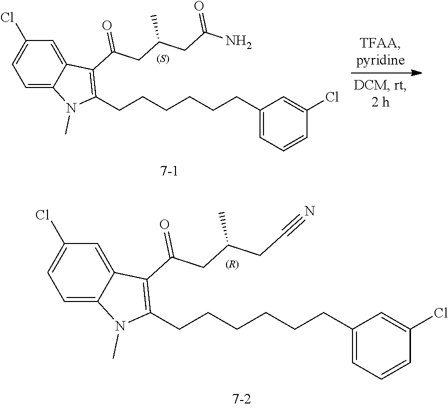

To a solution of 7-1 (160 mg, 0.33 mmol) and trifluoroacetic anhydride (104 mg, 0.50 mmol) in DCM (10 mL) was added pyridine (53 mg, 0.66 mmol) dropwise. Then the mixture was stirred at room temperature for 2 h until the reaction was complete (by LCMS). The reaction was quenched by adding water and extracted with DCM. The organic layer was combined, washed with brine and dried over anhydrous Na₂SO₄. The solvent was evaporated under reduced pressure and the crude was purified by silica gel chromatography (40% EtOAc/Hex) to afford 7-2 (110 mg, 71%).

### 7.3 The Synthesis of Compound I-13

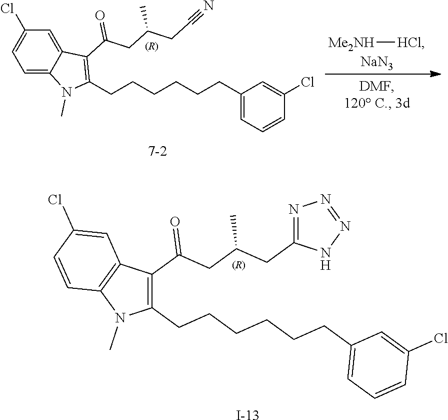

A mixture of 7-2 (110 mg, 0.24 mmol) and dimethylamine hydrochloride (192 mg, 2.35 mmol) in DMF, then sodium azide (153 mg, 2.35 mmol) was added under Ar protection. The reaction mixture was stirred at 120° C. for 3 d until the reaction was complete (by LCMS). The reaction was quenched by adding water and extracted with EtOAc. The organic layer was combined, washed with brine and dried over anhydrous Na₂SO₄. The solvent was evaporated under reduced pressure and the crude was purified by Prep-HPLC (Column: Waters X-Bridge C18 OBD 10 μm 19*250 mm; Flow Rate: 20 mL/min; solvent system: MeCN/(0.2% formic acid/water) gradient: MeCN: 50%˜95%; collection wavelength: 214 nm). The prep-HPLC fractions were concentrated at 35° C. under reduced pressure to remove MeCN, and the residue was lyophilizedto afford 1-13 (90 mg, 75%) as a brown solid. ¹H NMR (400 MHz, DMSO-d₆) δ 7.94 (d, J=2.0 Hz, 1H), 7.57 (d, J=8.4 Hz, 1H), 7.20-7.30 (m, 5H), 7.15 (d, J=7.6 Hz, 1H), 3.74 (s, 3H), 3.04-3.12 (m, 3H), 2.71-2.80 (m, 3H), 2.57 (t, J=7.6 Hz, 2H), 2.51-2.52 (m, 1H), 1.50-1.59 (, 4H), 1.39-1.44 (m, 2H), 1.31-1.36 (m, 2H), 0.90 (d, J=6.8 Hz, 3H). LC-MS m/z: 511.9 [M+H]⁺.

Additional exemplary compounds were prepared following methods substantially similar to the method described above and herein. Data for these compounds are provided in Tables 8 and 8a below.

TABLE 8
Characterization Data for Additional Exemplary Compounds

| Compound | | | |
|---|---|---|---|
| No. | Chemical Structure | M + 1 | ¹H NMR (400 MHz) |
| I-352 | 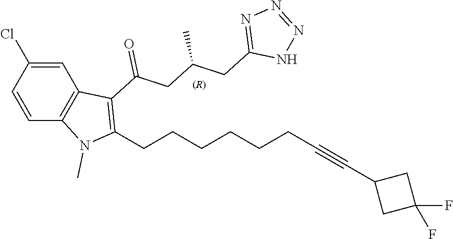 | 516.3 | ¹H NMR (400 MHz, DMSO-d6) δ 7.95 (d, J = 2.0 Hz, 1H), 7.59 (d, J = 8.8 Hz, 1H), 7.25 (dd, J = 8.8 Hz, 2.0 Hz, 1H), 3.76 (s, 3H), 3.13-3.09 (m, 2H), 3.06-3.00 (m, 2H), 2.95-2.85 (m, 5H), 2.69-2.60 (m, 1H), 2.55- 2.52 (m, 1H), 2.48-2.44 (m, 1H), 2.18-2.15 (m, 2H), 1.59-1.35 (m, 8H), 0.93 (d, J = 6.8 Hz, 3H) |
| I-353 | 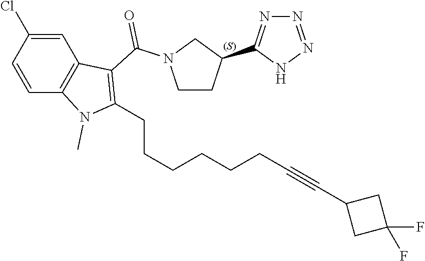 | 529.3 | ¹H NMR (400 MHz, DMSO-d₆) δ 16.28 (br., 1H), 7.52 (d, J = 8.8 Hz, 1H), 7.42 (d, J = 1.2 Hz, 1H), 7.15 (dd, J = 8.8 Hz, 2.0 Hz, 1H), 3.95-3.78 (m, 2H), 3.72 (s, 3H), 3.68-3.42 (m, 3H), 2.94-2.84 (m, 5H), 2.59- 2.53 (m, 1H), 2.49-2.43 (m, 1H), 2.42-2.29 (m, 1H), 2.27-2.08 (m, 3H), 1.58-1.48 (m, 2H), 1.43-1.22 (m, 6H). |
| I-354 | 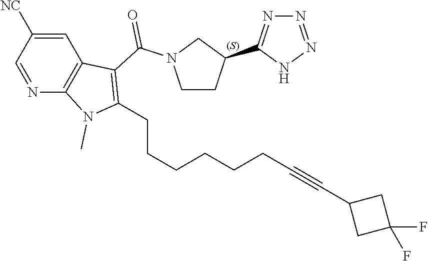 | 521.2 | ¹H NMR (400 MHz, DMSO-d₆) δ 16.25 (br., 1H), 8.66 (d, J = 2.0 Hz, 1H), 8.43 (d, J = 2.0 Hz, 1H), 3.90-3.86 (m, 1H), 3.83 (s, 3H), 3.81-3.41 (m, 4H), 2.92-2.84 (m, 5H), 2.55-2.45 (m, 3H), 2.28-2.13 (m, 3H), 1.64- 1.57 (m, 2H), 1.40-1.32 (m, 6H). |
| I-355 | 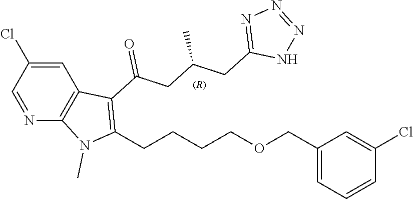 | 515.2 | ¹H NMR (400 MHz, DMSO-d6) δ 16.10 (br., 1H), 8.36-8.34 (m, 2H), 7.37-7.26 (m, 4H), 4.46 (s, 2H), 3.81 (s, 3H), 3.50 (t, J = 5.6 Hz, 2H), 3.21 (t, J = 7.6 Hz, 2H), 3.11- 2.96 (m, 3H), 2.89 (dd, J = 14.4, 8.0 Hz, 1H), 2.65-2.60 (m, 1H), 1.71-1.64 (m, 4H), 0.92 (d, J = 6.4 Hz, 3H). |
| I-356 | 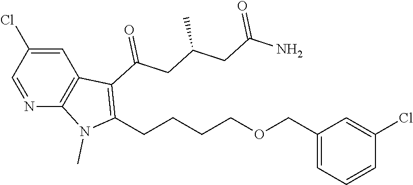 [SMILES: C[C@H](CC(N)=O)CC(=O)c1c(CCCCOCc2cccc(Cl)c2)n(C)c2ncc(Cl)cc12] | 490.2 | ¹H NMR (400 MHz, DMSO-d6) δ 8.38 (d, J = 2.4 Hz, 1H), 8.33 (d, J = 2.4 Hz, 1H), 7.39-7.27 (m, 5H), 6.79 (s, 1H), 4.47 (s, 2H), 3.80 (s, 3H), 3.50 (t, J = 5.6 Hz, 2H), 3.22 (t, J = 7.2 Hz, 2H), 3.04 (dd, J = 16.4 Hz, 5.2 Hz, 1H), 2.77 (dd, J = 16.0 Hz, 8.4 Hz, 1H), 2.50-2.45 (m, 1H), 2.17 (dd, J = 14.0 Hz, 6.4 Hz, 1H), 2.04 (dd, J = 14.0 Hz, 7.6 Hz, 1H), 1.72-1.66 (m, 4H), 0.92 (d, J = 6.4 Hz, 3H). |
| I-357 | 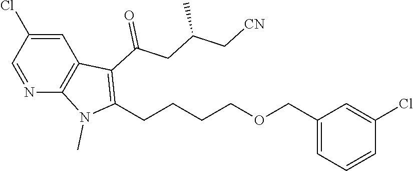 [SMILES: C[C@H](CC#N)CC(=O)c1c(CCCCOCc2cccc(Cl)c2)n(C)c2ncc(Cl)cc12] | 472.2 | ¹H NMR (400 MHz, DMSO-d6) δ 8.38- 8.34 (m, 2H), 7.39-7.27 (m, 4H), 4.47 (s, 2H), 3.81 (s, 3H), 3.51 (t, J = 5.6 Hz, 2H), 3.23 (t, J = 6.8 Hz, 2H), 3.04 (t, J = 6.0 Hz, 2H), 2.69-2.57 (m, 3H), 1.76-1.62 (m, 4H), 1.07 (d, J = 6.4 Hz, 3H). |
| I-358 | 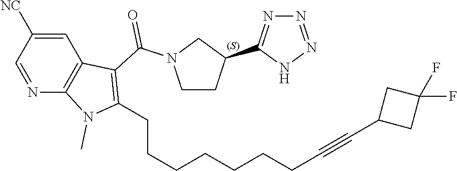 | 535.3 | ¹H NMR (400 MHz, DMSO-d6) δ 8.66 (d, J = 1.6 Hz, 1H), 8.43 (d, J = 2.0 Hz, 1H), 4.08-3.56 (m, 8H), 2.97-2.85 (m, 5H), 2.57- 2.52 (m, 1H), 2.48-2.32 (m, 2H), 2.14 (t, J = 6.4 Hz, 3H), 1.66-1.52 (m, 2H), 1.44-1.28 (m, 8H). |
| I-359 | 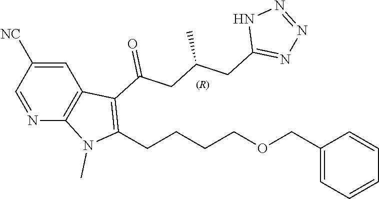 | 472.3 | ¹H NMR (400 MHz, DMSO-d6) δ 8.79 (d, J = 2.0 Hz, 1H), 8.72 (d, J = 1.6 Hz, 1H), 7.31-7.21 (m, 5H), 4.42 (s, 2H), 3.82 (s, 3H), 3.46 (t, J = 5.6 Hz, 2H), 3.20 (t, J = 7.2 Hz, 2H), 3.12 (dd, J = 16.8 Hz, 4.4 Hz, 1H), 3.04-2.97 (m, 2H), 2.85 (dd, J = 14.8 Hz, 8.0 Hz, 1H), 2.64-2.57 (m, 1H), 1.66-1.65 (m, 4H), 0.90 (d, J = 6.4 Hz, 3H). |
| I-360 | 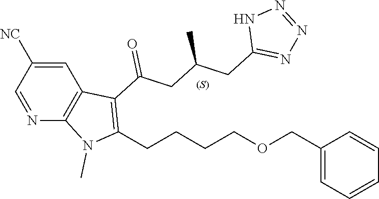 | 472.3 | ¹H NMR (400 MHz, DMSO-d6) δ 16.07 (br., 1H), 8.79 (d, J = 1.6 Hz, 1H), 8.72 (d, J = 2.0 Hz, 1H), 7.31-7.23 (m, 5H), 4.42 (s, 2H), 3.82 (s, 3H), 3.46 (t, J = 5.2 Hz, 2H), 3.20 (t, J = 6.4 Hz, 2H), 3.11 (dd, J = 17.2 Hz, 5.6 Hz, 1H), 3.05-2.98 (m, 2H), 2.86 (dd, J = 14.8 Hz, 8.0 Hz, 1H), 2.64-2.56 (m, 1H), 1.66-1.65 (m, 4H), 0.90 (d, J = 6.8 Hz, 3H). |

TABLE 9a
Characterization Data for Additional Exemplary Compounds

| Compound | Rt (Min) | Rt (Min) |
|---|---|---|
| No. | (LCMS) | (HPLC) |
| I-352 | 2.066 | 10.485 |
| I-353 | 1.915 | 8.989 |
| I-354 | 1.804 | 8.824 |
| I-355 | 2.013 | 10.202 |
| I-356 | 1.998 | 10.437 |
| I-357 | 2.040 | 11.387 |
| I-358 | 1.884 | 9.320 |
| I-359 | 1.837 | 9.012 |
| I-360 | 1.837 | 9.012 |

### Example 8: Synthesis of Compound I-14

### Synthetic Scheme for Compound I-14

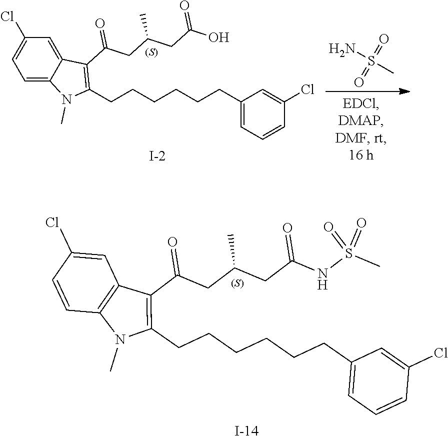

To a mixture of I-2 (250 mg, 0.51 mmol), methanesulfonamide (96 mg, 0.61 mmol), EDCI (57 mg, 0.30 mmol) and DMAP (61 mg, 0.50 mmol) in DMF (5 mL), then the mixture was stirred at room temperature overnight. The reaction was quenched by adding water and extracted with EtOAc. The organic layer was combined, washed with brine and dried over anhydrous Na₂SO₄. The solvent was evaporated under reduced pressure and the crude was purified by Prep-HPLC to afford I-14 (27 mg, 10%) as a white solid. ¹H NMR (400 MHz, DMSO-d₆) δ 7.95 (d, J=2.0 Hz, 1H), 7.59 (d, J=8.8 Hz, 1H), 7.21-7.31 (m, 5H), 7.16 (d, J=7.6 Hz, 1H), 3.75 (s, 3H), 3.31 (s, 1H), 3.11-3.16 (m, 5H), 2.96 (dd, J=16.4 Hz, 5.6 Hz, 1H), 2.84 (dd, J=16.4 Hz, 7.6 Hz, 1H), 2.58 (t, J=7.6 Hz, 2H), 2.33-2.40 (m, 1H), 2.20 (dd, J=14.8 Hz, 7.6 Hz, 1H), 1.52-1.62 (m, 4H), 1.40-1.48 (m, 2H), 1.32-1.37 (m, 2H), 0.95 (d, J=6.8 Hz, 3H). LC-MS m/z: 564.8 [M+H]⁺.

Additional exemplary compounds were prepared following methods substantially similar to the method described above and herein. Data for these compounds are provided in Tables 10 and 10a below.

TABLE 10
Characterization Data for Additional Exemplary Compounds

| Compound | | | |
|---|---|---|---|
| No. | Chemical Structure | M + 1 | ¹H NMR (400 MHz) |
| I-361 | 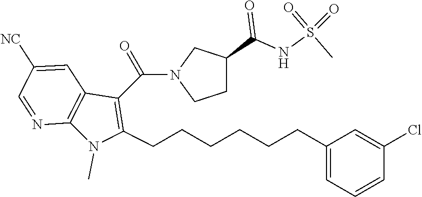 [SMILES: Cn1c(CCCCCCc2cccc(Cl)c2)c(C(=O)N2CC[C@H](C(=O)NS(C)(=O)=O)C2)c2cc(C#N)cnc21] | 570.30 | ¹H NMR (400 MHz, DMSO-d₆) δ 11.86 (s, 1H), 8.65 (d, J = 2.0 Hz, 1H), 8.39 (s, 1H), 7.30-7.14 (m, 4H), 3.82 (s, 3H), 3.79-3.40 (m, 4H), 3.28-3.20 (m, 3H), 3.19-3.05 (m, 1H), 2.92 (t, J = 7.2 Hz, 2H), 2.58-2.54 (m, 2H), 2.20-1.95 (m, 2H), 1.65-1.50 (m, 4H), 1.41-1.25 (m, 4H). |
| I-362 | 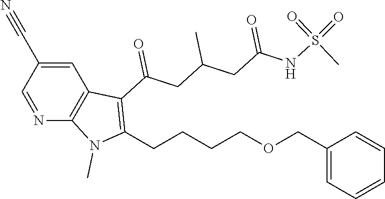 [SMILES: CC(CC(=O)NS(C)(=O)=O)CC(=O)c1c(CCCCOCc2ccccc2)n(C)c2ncc(C#N)cc12] | 525.3 | ¹H NMR (400 MHz, DMSO-d₆) δ 11.75 (s, 1H), 8.81 (d, J = 1.6 Hz, 1H), 8.74 (d, J = 1.6 Hz, 1H), 7.35-7.25 (m, 5H), 4.46 (s, 2H), 3.84 (s, 3H), 3.50 (t, J = 5.6 Hz, 2H), 3.28-3.21 (m, 5H), 3.08 (dd, J = 17.2, 5.6 Hz, 1H), 2.93 (dd, J = 16.8, 7.6 Hz, 1H), 2.57-2.53 (m, 1H), 2.43 (dd, J = 14.8, 5.6 Hz, 1H), 2.24 (dd, J = 15.2, 7.6 Hz, 1H), 1.74-1.68 (m, 4H), 0.96 (d, J = 6.8 Hz, 3H). |
| I-363 | 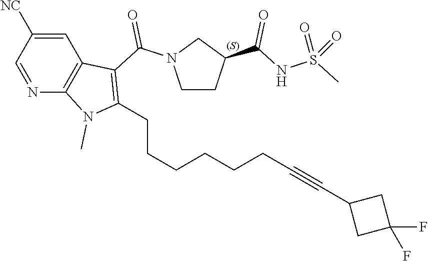 | 574.3 | ¹H NMR (400 MHz, DMSO-d₆) δ 8.65 (d, J = 2.0 Hz, 1H), 8.40 (s, 1H), 7.08 (br., 1H), 3.83 (s, 3H), 3.75-3.33 (m, 4H), 3.17-2.86 (m, 9H), 2.58-2.51 (m, 2H), 2.17-1.92 (m, 4H), 1.62-1.59 (m, 2H), 1.41-1.33 (m, 6H). |
| I-364 | 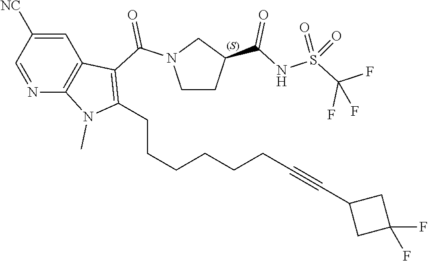 | 628.2 | ¹H NMR (400 MHz, DMSO-d₆) δ 8.64 (d, J = 2.0 Hz, 1H), 8.36 (s, 1H), 5.36- 4.23 (m, 5H), 3.82 (s, 3H), 3.67 (br., 2H), 2.96-2.86 (m, 5H), 2.58-2.51 (m, 2H), 2.18-1.91 (m, 4H), 1.65-1.53 (m, 2H), 1.45-1.30 (m, 6H), 1.23 (s, 1H). |

TABLE 10a
Additional Characterization Data for Additional Exemplary Compounds

| Compound | Rt (Min) | Rt (Min) |
|---|---|---|
| No. | (LCMS) | (HPLC) |
| I-361 | 1.922 | 9.560 |
| I-362 | 1.868 | 9.191 |
| I-363 | 1.761 | 9.007 |
| I-364 | 1.845 | 9.227 |

### Example 9: Synthesis of Compound I-23

### Synthetic Scheme for Compound 1-23

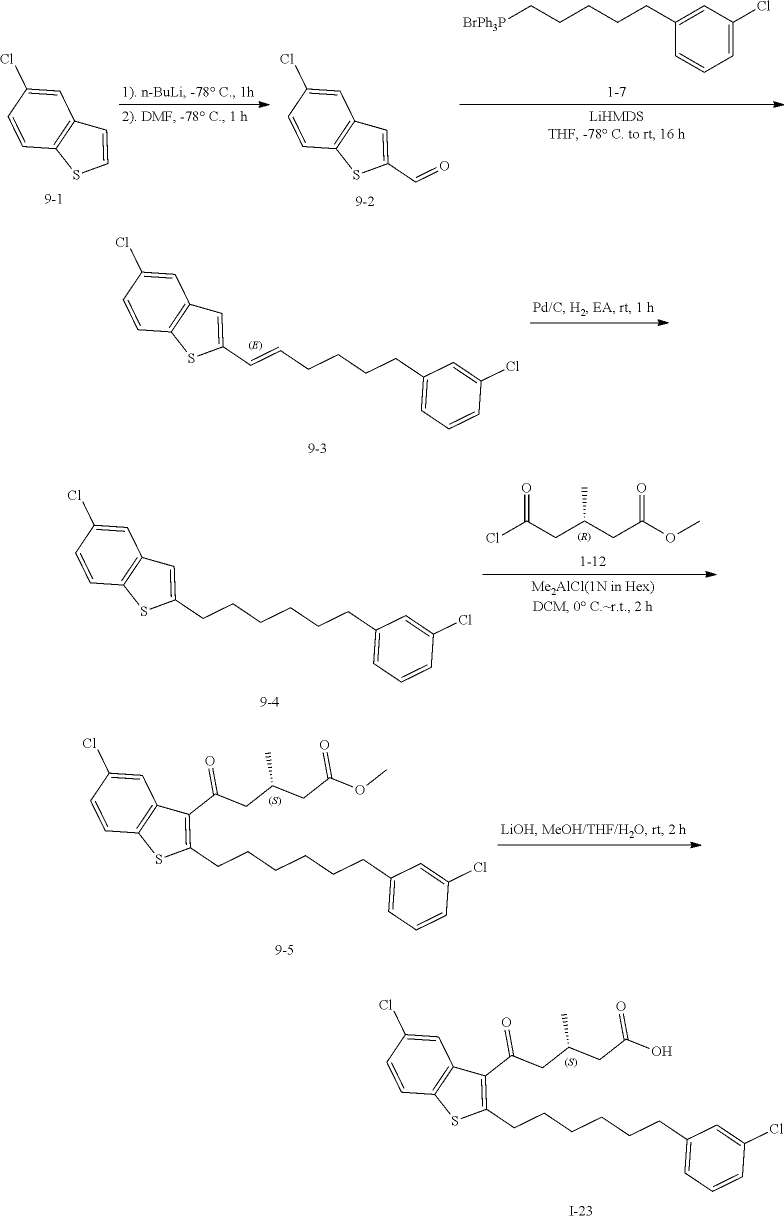

### 9.1 The Synthesis of Intermediate 9-2

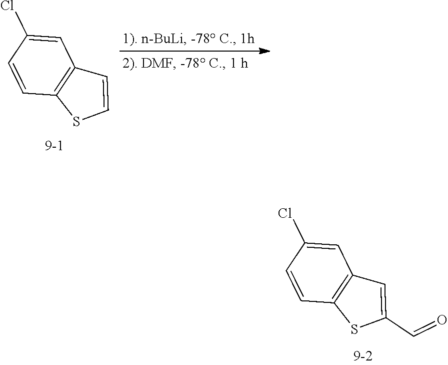 [SMILES: Clc1ccc2sccc2c1 ; O=Cc1cc2cc(Cl)ccc2s1]

To a solution of 9-1 (5.0 g, 29.6 mmol) in THF (100 mL) was added n-BuLi (2.5 M in THF, 13 mL, 32.6 mmol) at −78° C. The mixture was stirred for 1 h at −78° C. and DMF (4.6 mL, 59.2 mmol) in THF (20 ml) was added dropwise. The mixture was stirred for 1 h at −78° C. Saturated NH₄Cl solution was added at 0° C. and the reaction mixture was extracted with EtOAc, the combined organic layer was washed with brine, dried over anhydrous Na₂SO₄ and evaporated under reduced pressure. The crude was purified by silica gel chromatography ((PE/EA=9/1) to afford 9-2 (5.5 g, yield: 95%) as a light yellow solid. ¹H NMR (400 MHz, CDCl₃) δ 10.11 (s, 1H), 7.97 (s, 1H), 7.93 (d, J=2.0 Hz, 1H), 7.83 (d, J=8.8 Hz, 1H), 7.47 (dd, J=8.8 Hz, 2.0 Hz, 1H).

### 9.2 The Synthesis of Intermediate 9-3

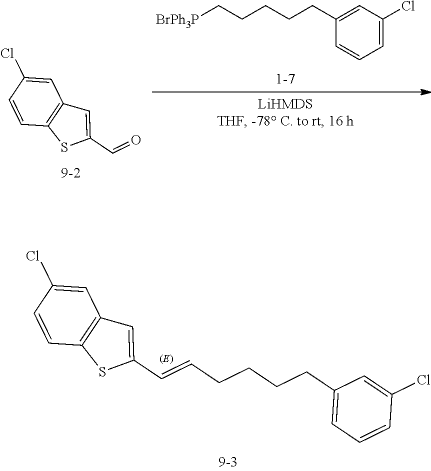

To a suspension of 1-7 (12.0 g, 23.1 mmol) in THF (100 mL) was added LiHMDS (1.0 M in THF, 23 mL, 23 mmol) at −78° C. The mixture was stirred for 30 min and 9-2 (3.0 g, 15.4 mmol) in THF (20 ml) was added dropwise. The reaction mixture was allowed to warm to rt and stirred overnight. Saturated NH₄Cl solution was added at 0° C. and the mixture was acidified with HCl (1.0 M) to pH˜3, extracted with EtOAc, the combined organic layer was washed with brine, dried over anhydrous Na₂SO₄ and evaporated under reduced pressure. The crude was purified by silica gel chromatography (5%-10% EtOAc/PE) to afford 9-3 (4.3 g, yield: 78%) as a light yellow oil.

### 9.3 The Synthesis of Intermediate 9-4

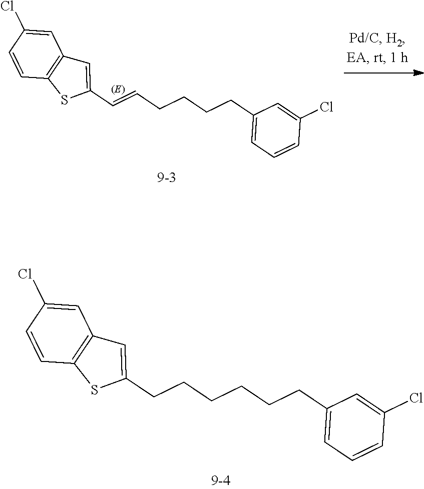 [SMILES: Clc1cccc(CCCC/C=C/c2cc3cc(Cl)ccc3s2)c1 ; Clc1cccc(CCCCCCc2cc3cc(Cl)ccc3s2)c1]

To a stirred solution of 9-3 (4.3 g, 12.1 mmol) in EtOAc (100 mL) was added 10% Pd/C (500 mg) under H₂ atmosphere (1.0 atm). The reaction mixture was stirred at rt for 1 h until the reaction was complete (by LCMS) then filtered. The residue was washed with EtOAc and the combined filtrate was concentrated under reduced pressure and the crude product was purified by silica gel chromatography (PE/EA=100/1) to afford 9-4 (3.9 g, yield: 88%) as a white solid. ¹H NMR (400 MHz, CDCl₃) δ 7.62-7.66 (m, 2H), 7.14-7.22 (m, 4H), 7.03 (d, J=7.2 Hz, 1H), 6.91 (s, 1H), 2.88 (t, J=7.2 Hz, 2H), 2.57 (t, J=7.6 Hz, 2H), 1.71-1.75 (m, 2H), 1.59-1.63 (m, 2H), 1.26-1.43 (m, 4H).

### 9.4 The Synthesis of Intermediate 9-5

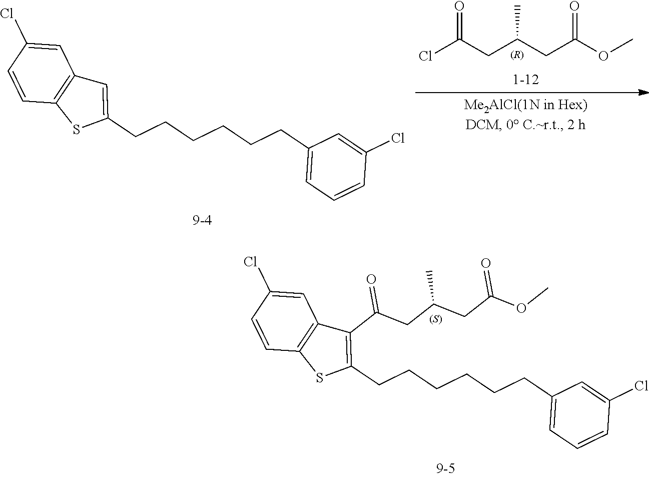

To a stirred solution of 9-4 (500 mg, 1.4 mmol) in dichloromethane (10 mL) was added AlCl₃ (373 mg, 2.8 mmol) at 0° C. After 45 min, 1-12 (167.0 mg, 0.9 mmol) in CH₂Cl₂ (5 mL) was added dropwise and the reaction mixture was stirred at room temperature for another 2 h. The reaction was quenched by adding water and extracted with EtOAc. The organic layer was combined, washed with brine and dried over Na₂SO₄. The solvent was evaporated under reduced pressure and the crude was purified by silica gel chromatography (15% EtOAc/Hex) to afford 9-5 (300 mg, 63%) as yellow oil. LC-MS m/z: 505.3 [M]⁺.

### 9.5 The Synthesis of Compound I-23

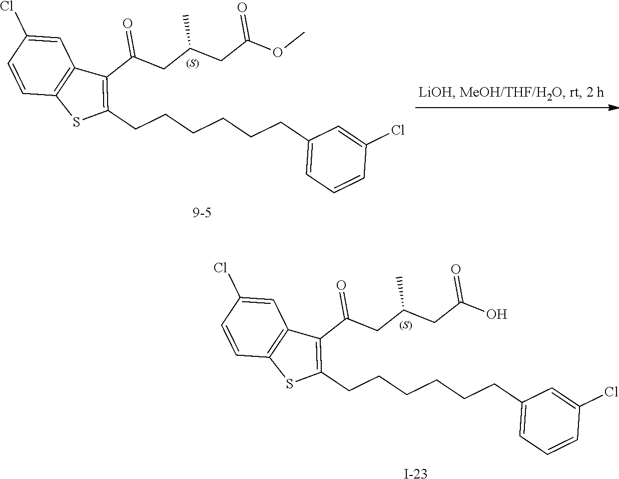

To a stirred solution of 9-5 (280 mg, 0.6 mmol) in MeOH/THF/H₂O (5 mL/2 mL/1 mL) was added lithium hydroxide (139 mg, 3.3 mmol). The reaction mixture was stirred at rt for 2 h until the reaction was complete (by LCMS). Saturated NH₄Cl solution was added at 0° C. and the mixture was acidified with HCl (1.0 M) to pH ˜3.0 then extracted with EtOAc, the combined organic layer was washed with brine, dried over anhydrous Na₂SO₄ and evaporated under reduced pressure. The crude was purified by Prep-HPLC (Column: Waters X-Bridge C18 OBD 10m 19*250 mm; Flow Rate: 20 mL/min; solvent system: MeCN/(0.2% formic acid/water) gradient: MeCN: 50%˜95%; collection wavelength: 214 nm). The prep-HPLC fractions were concentrated at 35° C. under reduced pressure to remove MeCN, and the residue was lyophilized to afford I-23 (100 mg, yield: 37%) as yellow oil. ¹H NMR (400 MHz, DMSO-d₆) δ 12.14 (s, 1H), 7.99-8.01 (m, 2H), 7.42 (dd, J=8.8 Hz, 2.0 Hz, 1H), 7.21-7.30 (m, 3H), 7.14 (d, J=7.2 Hz, 1H), 3.04-3.10 (m, 3H), 2.88 (dd, J=16.8 Hz, 8.0 Hz, 1H), 2.49-2.58 (m, 4H), 2.36 (dd, J=15.6 Hz, 4.0 Hz, 1H), 2.19 (dd, J=15.6 Hz, 8.0 Hz, 1H), 1.67-1.71 (m, 2H), 1.53-1.57 (m, 2H), 1.29-1.42 (m, 4H), 0.96 (d, J=6.8 Hz, 3H). LC-MS m/z: 492.8 [M+H]⁺.

Additional exemplary compound, I-21, was prepared following methods substantially similar to the method described above and herein.

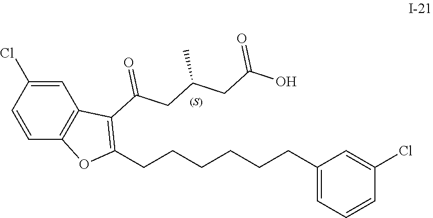

¹H NMR (400 MHz, DMSO-d₆) δ 12.11 (s, 1H), 8.00 (d, J=2.0 Hz, 1H), 7.66 (d, J=8.4 Hz, 1H), 7.39 (dd, J=8.8, 2.4 Hz 1H), 7.30-7.20 (m, 1H), 7.14 (d, J=7.2 Hz, 3H), 3.13 (t, J=7.6 Hz, 2H), 3.06 (dd, J=17.2, 6.0 Hz, 1H), 2.90 (dd, J=17.2, 7.6 Hz, 1H), 2.56 (t, J=7.6 Hz, 2H), 2.48-2.46 (m, 1H), 2.38 (dd, J=15.6, 6.0 Hz, 1H), 2.19 (dd, J=15.6, 8.0 Hz, 1H), 1.74-1.69 (m, 2H), 1.57-1.52 (m, 2H), 1.40-1.30 (m, 4H), 0.97 (d, J=6.4 Hz, 3H). LC-MS m/z: 475.0 [M+H]⁺.

### Example 10: Synthesis of Compound I-162

### Synthetic Scheme for Compound I-162

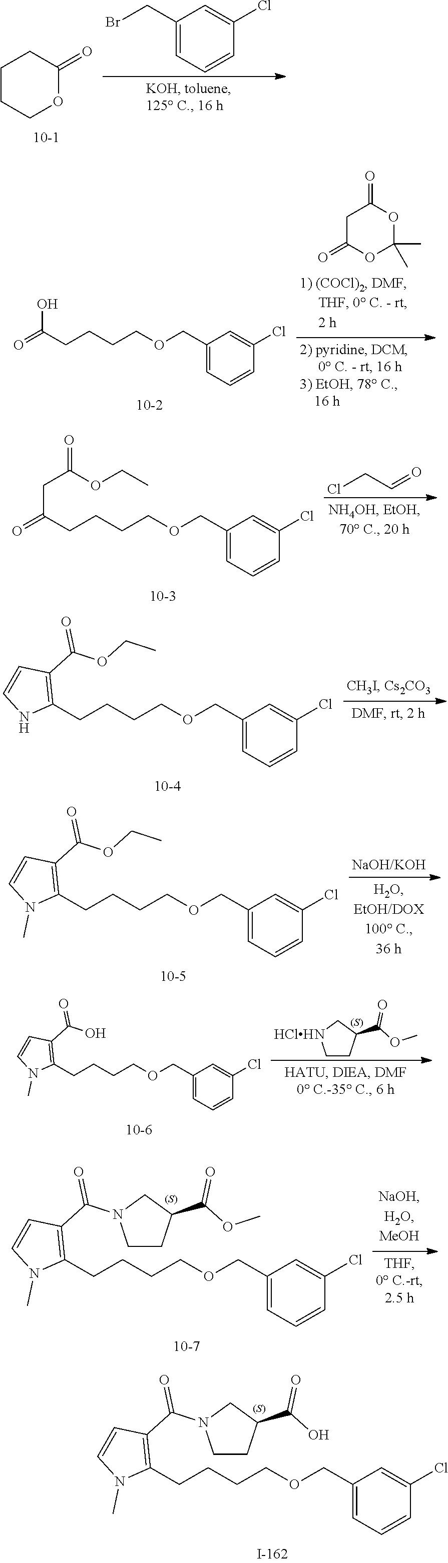

### 10.1 The Synthesis of Intermediate 10-2

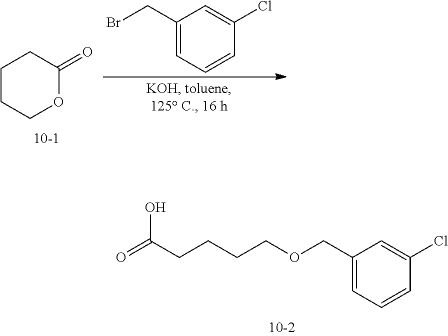

To a solution of tetrahydro-2H-pyran-2-one (5.0 g, 50.0 mmol) in toluene (50 mL) was added 1-(bromomethyl)-3-chlorobenzene (30.6 g, 149.9 mmol) and KOH (15.8 g, 281.9 mmol) at room temperature. The reaction mixture was stirred at 125° C. for 16 h. After completion, it was allowed to cool to room temperature and quenched with ice-water (200 mL). After standing for a few minutes, the organic layer was separated, and the aqueous phase was washed with MTBE (30 mL×3). Then the aqueous phase was cooled to 0° C. and adjusted pH to 3˜4 with HCl (3 M). The suspension was extracted with EtOAc (60 mL×4), dried over anhydrous Na₂SO₄ and concentrated under reduced pressure to afford 10-2 (10.7 g, yield: 83%) as light yellow oil.

### 10.2 The Synthesis of Intermediate 10-3

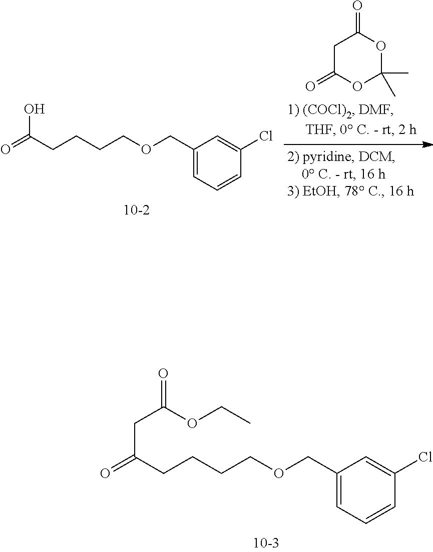

To a stirred solution of 10-2 (10.0 g, 41.3 mmol) in anhydrous THF (110 mL), was added (COCI)₂ (10.4 g, 82.6 mmol) and anhydrous DMF (0.1 mL) at 0° C. The mixture was allowed to room temperature and stirred for another 2 h. Then it was concentrated under reduce pressure, dissolved in anhydrous DCM and added to a solution of 2,2-dimethyl-1,3-dioxane-4,6-dione (5.95 g, 41.3 mmol) and pyridine (6.5 g, 82.6 mmol) in anhydrous DCM (110 mL) at 0° C. The reaction mixture was stirred at room temperature for 16 h. The solution was acidified with 0.5 M HCl, extracted with EtOAc (50 mL×3), dried over Na₂SO₄ and concentrated to yellow oil. Added EtOH (120 mL) and stirred the solution at 78° C. for 16 h. Upon completion, it was cooled to room temperature, evaporated under reduced pressure and purified by silica gel chromatography (PE/EA ˜10/1) to afford 10-3 (9.7 g, yield: 75%) as yellow oil.

### 10.3 The Synthesis of Intermediate 10-4

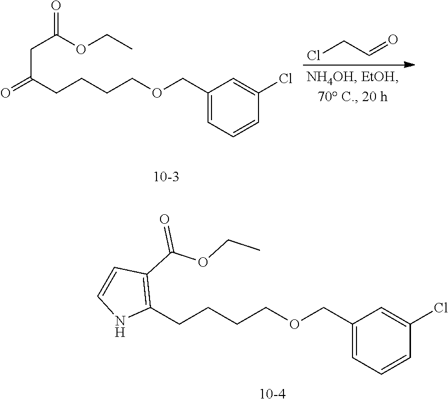

To a stirred solution of 10-3 (2.1 g, 6.7 mmol) in EtOH (6 mL), was added NH₄OH (8 mL, 53.8 mmol, 28% in water) and 2-chloroacetaldehyde (1.2 mL, 6.7 mmol, 40% in water) at room temperature. The reaction mixture was allowed to heat to 70° C. and stirred for 20 h. After the reaction was complete (by LCMS), the mixture was cooled to room temperature and adjusted pH to 4 with HCl aqueous. The suspension was washed with water (100 mL), extracted with EtOAc (50 mL×3), dried over anhydrous Na₂SO₄ and evaporated under reduced pressure to afford crude product. The crude oil was purified by silica gel chromatography (PE/EA ˜4/1) to afford 10-4 (1.05 g, 46%) as yellow oil. ¹H NMR (400 MHz, DMSO-d₆) δ 11.19 (s, 1H), 7.39-7.32 (m, 3H), 7.28-7.26 (m, 1H), 6.61 (t, J=2.4 Hz, 1H), 6.32 (t, J=2.8 Hz, 1H), 4.44 (s, 2H), 4.13 (q, J=14.0, 6.8 Hz, 2H), 3.43 (t, J=6.0 Hz, 2H), 2.86 (t, J=7.2 Hz, 2H), 1.67-1.59 (m, 2H), 1.57-1.50 (m, 2H), 1.23 (t, J=7.2 Hz, 3H).

### 10.4 The Synthesis of Intermediate 10-5

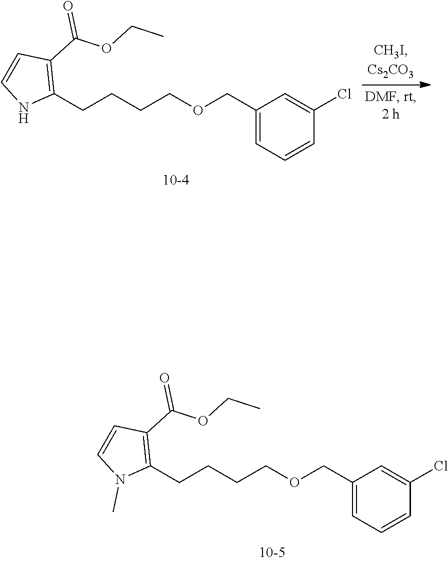 [SMILES: CCOC(=O)c1cc[nH]c1CCCCOCc1cccc(Cl)c1 ; CCOC(=O)c1ccn(C)c1CCCCOCc1cccc(Cl)c1]

To a solution of 10-4 (200 mg, 0.6 mmol) and Cs₂CO₃ (855 mg, 2.6 mmol) in DMF (4 mL) was added CH₃I (425 mg, 3.0 mmol) under N₂ atmosphere at 0° C. Then the mixture was allowed to warm to room temperature and stirred for 2 h. Water (40 mL) was added to the mixture and it was extracted with EtOAc (15 mL×3). The organic layer was washed with brine (30 mL), dried over anhydrous Na₂SO₄, concentrated in vacuo to give 10-5 (150 mg, yield: 70%) as yellow oil.

### 10.5 The Synthesis of Intermediate 10-6

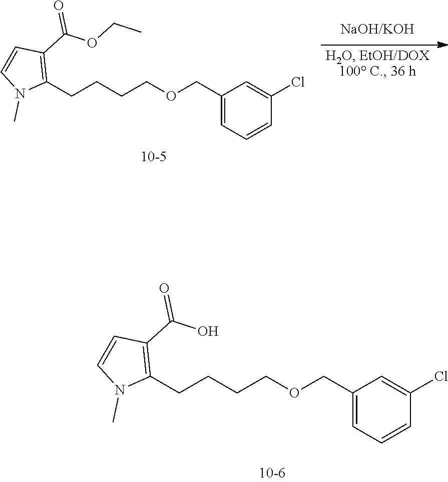 [SMILES: CCOC(=O)c1ccn(C)c1CCCCOCc1cccc(Cl)c1 ; "H2O, EtOH/DOX100° C., 36 h" ; "NaOH/KOH" ; Cn1ccc(C(=O)O)c1CCCCOCc1cccc(Cl)c1]

To a solution of 10-5 (150 mg, 0.4 mmol) in dioxane (1 mL) and EtOH (0.3 mL), was added KOH (120 mg, 2.15 mmol) and NaOH (150 mg, 3.87 mmol) and H₂O (1 mL) at room temperature. The mixture was stirred at 100° C. for 36 h. Then it was adjusted the pH to 3 with 3 N HCl aqueous, extracted with EtOAc (15 mL×3). The combined organic layer was dried over anhydrous Na₂SO₄ and concentrated in vacuum to give a crude 10-6 (230 mg, 111%) as yellow oil.

### 10.6 The Synthesis of Intermediate 10-7

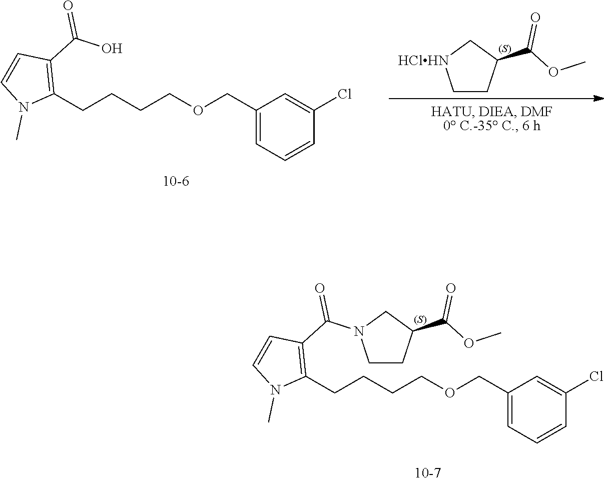

To a stirred solution of 10-6 (210 mg, 0.65 mmol) in anhydrous DMF (6 mL) was added HATU (373 mg, 0.98 mmol). The mixture was stirred at room temperature for 10 min, a solution with (S)-methyl pyrrolidine-3-carboxylate hydrochloride (119 mg, 0.72 mmol) and DIPEA (675 mg, 5.23 mmol) in anhydrous DMF (4 mL) was added. The reaction mixture was stirred at 35° C. for 6 h. Upon completion, ice-water (150 mL) was added, and the suspension was extracted with EtOAc (15 mL×3), dried over Na₂SO₄ and evaporated under reduced pressure to afford 10-7 (330 mg, yield: 106%) as crude yellow oil. 10.7 The Synthesis of Compound I-162

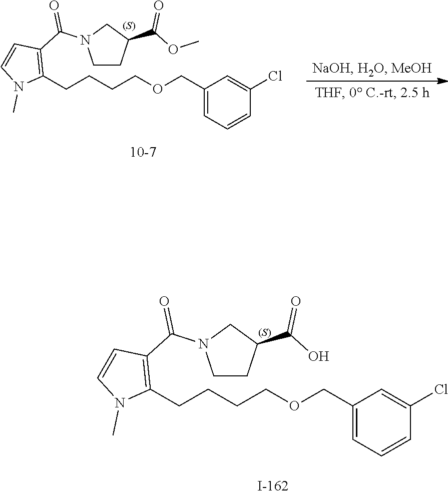

To a solution of 10-7 (300 mg, 0.69 mmol) in THF (6 mL) was added MeOH (3.4 mL) and NaOH (270 mg in 1.7 mL H₂O, 6.94 mmol) at 0° C. The mixture was stirred at room temperature for 2.5 h. Then, water (10 mL) was added, and adjusted pH to 3˜4 with HCl (2 N). The suspension was extracted with EtOAc (15 mL×3), dried over anhydrous Na₂SO₄ and the solvent was concentrated under reduced pressure. The crude was purified by Prep-HPLC (ACN/H₂O) to afford 1-162 (106.91 mg, yield: 34%) as yellow oil. ¹H NMR (400 MHz, DMSO-d₆) δ 12.42 (br., 1H), 7.39-7.33 (m, 3H), 7.28-7.27 (m, 1H), 6.61 (d, J=2.0 Hz, 1H), 6.16 (d, J=2.0 Hz, 1H), 4.44 (s, 2H), 3.65-3.63 (m, 2H), 3.51 (s, 3H), 3.43-3.41 (m, 4H), 3.05-2.99 (m, 1H), 2.78-2.75 (m, 2H), 2.09-1.94 (m, 2H), 1.58-1.46 (m, 4H). LC-MS m/z: 419.2 [M+H]⁺.

Additional exemplary compounds were prepared following methods substantially similar to the method described above and herein. Data for these compounds are provided in Table 11 below.

TABLE 11
Characterization Data for Additional Exemplary Compounds

| Compound | | | |
|---|---|---|---|
| No | Chemical Structure | M + 1 | ¹H NMR (400 MHz) |
| I-166 | 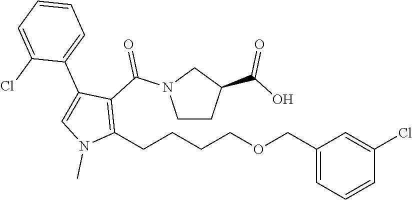 [SMILES: Cn1cc(-c2ccccc2Cl)c(C(=O)N2CC[C@H](C(=O)O)C2)c1CCCCOCc1cccc(Cl)c1] | 529.2 | ¹H NMR (400 MHz, DMSO-d₆) δ 7.44 (dd, J = 7.6, 1.2 Hz, 1H), 7.39- 7.32 (m, 3H), 7.28-7.15 (m, 4H), 6.91 (s, 1H), 4.45 (s, 2H), 3.58 (s, 3H), 3.42-3.40 (m, 5H), 2.99-2.90 (m, 1H), 2.86-2.76 (m, 1H), 2.67- 2.63 (m, 2H), 1.91-1.64 (m, 2H), 1.56-1.48 (m, 4H). |
| I-167 | 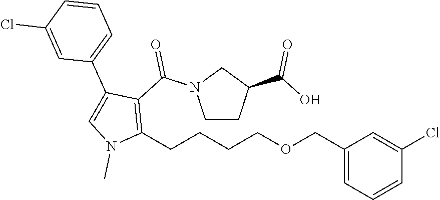 [SMILES: Cn1cc(-c2cccc(Cl)c2)c(C(=O)N2CC[C@H](C(=O)O)C2)c1CCCCOCc1cccc(Cl)c1] | 529.2 | ¹H NMR (400 MHz, DMSO-d₆) δ 12.43 (br., 1H), 7.39-7.26 (m, 6H), 7.22-7.17 (m, 2H), 7.08 (s, 1H), 4.45 (s, 2H), 3.58-3.42 (m, 7H), 2.97-2.77 (m, 3H), 2.61-2.55 (m,1H), 2.20- 1.60 (m, 3H), 1.58-1.47 (m, 4H). |
| I-171 | 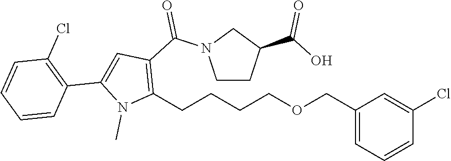 [SMILES: Cn1c(-c2ccccc2Cl)cc(C(=O)N2CC[C@H](C(=O)O)C2)c1CCCCOCc1cccc(Cl)c1] | 529.2 | ¹H NMR (400 MHz, DMSO-d₆) δ 12.32 (br., 1H), 7.44 (dd, J = 7.6, 1.2 Hz, 1H), 7.39-7.32 (m, 3H), 7.28- 7.15 (m, 4H), 6.91 (s, 1H), 4.45 (s, 2H), 3.58 (s, 3H), 3.48-3.42 (m, 3H), 3.31-3.28 (m, 1H), 3.04-2.64 (m, 5H), 1.86-1.79 (m, 2H), 1.56-1.48 (m, 4H). |
| I-172 | 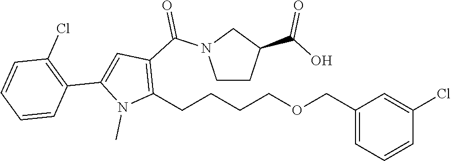 [SMILES: Cn1c(-c2ccccc2Cl)cc(C(=O)N2CC[C@H](C(=O)O)C2)c1CCCCOCc1cccc(Cl)c1] | 529.2 | ¹H NMR (400 MHz, CDCl₃) δ 7.40- 7.31 (m, 4H), 7.19-7.13 (m, 4H), 6.62 (d, J = 3.6 Hz, 1H), 4.50-4.47 (m, 2H), 3.91-3.84 (m, 2H), 3.55- 3.41 (m, 7H), 2.95-2.90 (m, 2H), 2.88-2.59 (m, 2H), 2.21 (br., 1H), 1.88-1.83 (m, 1H), 1.63-1.59 (m, 4H). |
| I-191 | 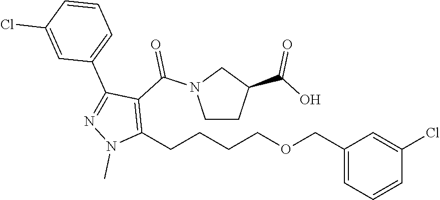 [SMILES: Cn1nc(-c2cccc(Cl)c2)c(C(=O)N2CC[C@H](C(=O)O)C2)c1CCCCOCc1cccc(Cl)c1] | 530.1 | ¹H NMR (400 MHz, DMSO-d₆) δ 12.51 (s, 1H), 7.58-7.56 (m, 1H), 7.48-7.33 (m, 6H), 7.29-7.27 (m, 1H), 4.45 (s, 2H), 3.82 (d, J = 2.0 Hz, 3H), 3.70-3.50 (m, 2H), 3.46-3.42 (m, 2H), 3.20-2.83 (m, 3H), 2.71- 2.65 (m, 2H), 2.04-1.79 (m, 2H), 1.62-1.51 (m, 4H). |
| I-192 | 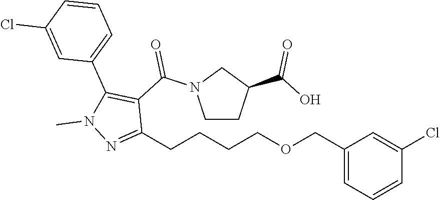 [SMILES: Cn1nc(CCCCOCc2cccc(Cl)c2)c(C(=O)N2CC[C@H](C(=O)O)C2)c1-c1cccc(Cl)c1] | 530.2 | ¹H NMR (400 MHz, DMSO-d₆) δ 7.52-7.48 (m, 3H), 7.40-7.33 (m, 4H), 7.28 (d, J = 7.2 Hz, 1H), 4.45 (s, 2H), 3.74 (s, 3H), 3.57-3.40 (m, 4H), 3.05-2.78 (m, 3H), 2.56 (t, J = 5.6 Hz, 2H), 1.96-1.73 (m, 2H), 1.65-1.53 (m, 4H). |

### Example 11: Synthesis of compounds I-105 and I-106

### Synthetic Scheme for Compounds I-105 and I-106

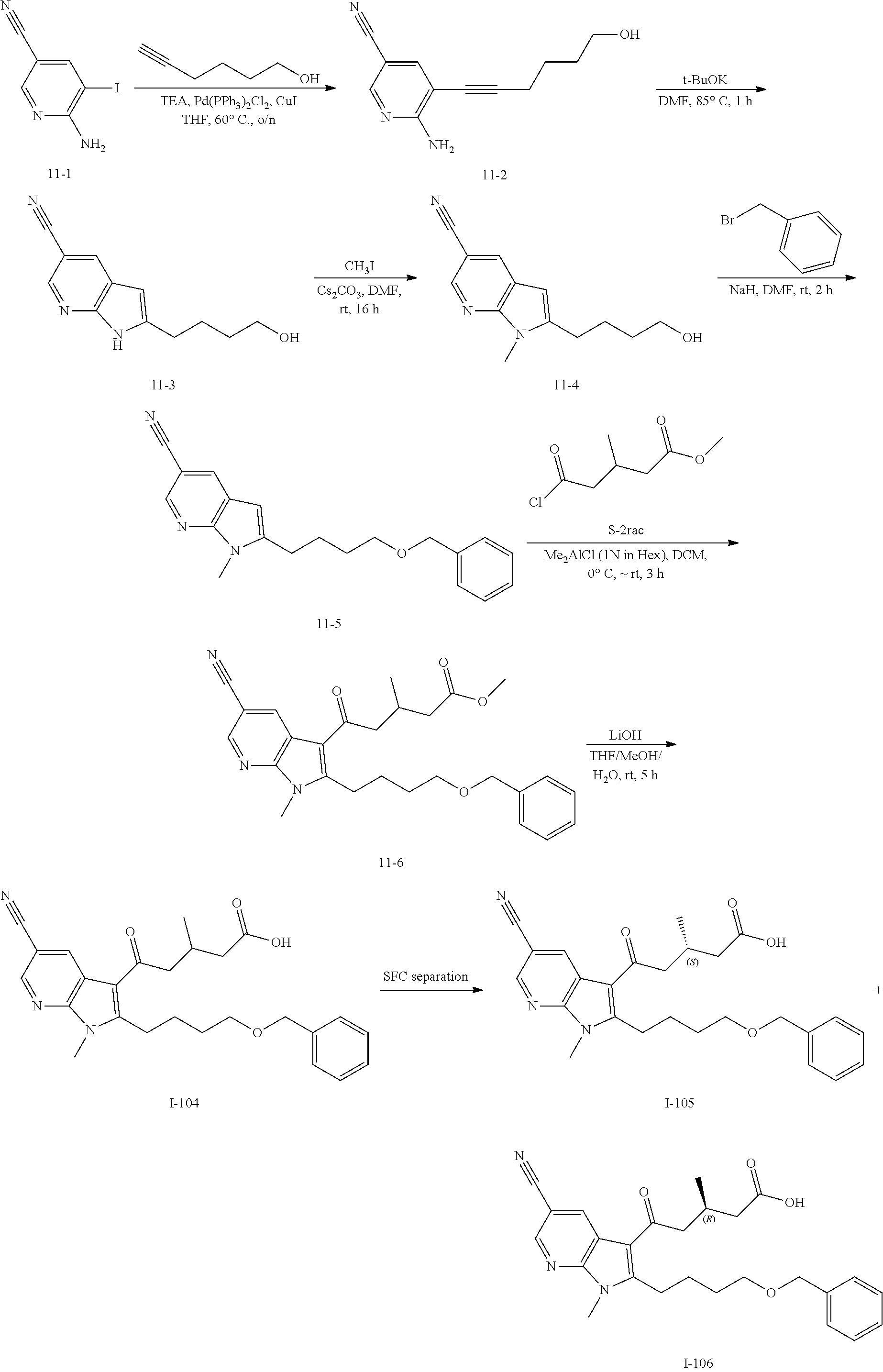

### 11.1 The Synthesis of Intermediate 11-2

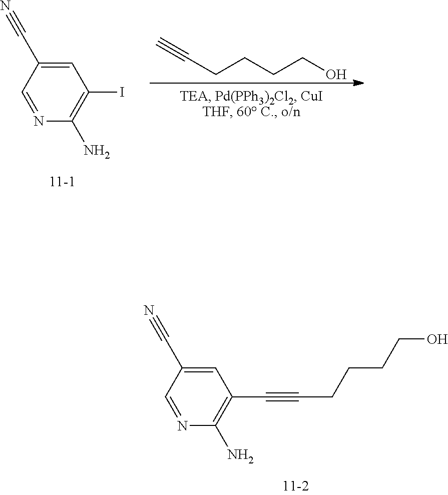

To a stirred solution of 6-amino-5-iodonicotinonitrile (11-1, 30.0 g, 122.4 mmol) in THF (200 mL) and TEA (100 mL) was added hex-5-yn-1-ol (13.2 g, 134.7 mmol), Pd(PPh₃)₂Cl₂ (4.3 g, 6.1 mmol) and CuI (1.2 g, 6.1 mmol). The reaction mixture was stirred at 60° C. overnight under nitrogen atmosphere. The mixture was filtered through a pad of diatomite, then H₂O (500 mL) and EA (500 mL) were added and the organic phase was collected and evaporated under reduced pressure to give 11-2 (28.0 g, crude) as yellow oil.

### 11.2 The Synthesis of Intermediate 11-3

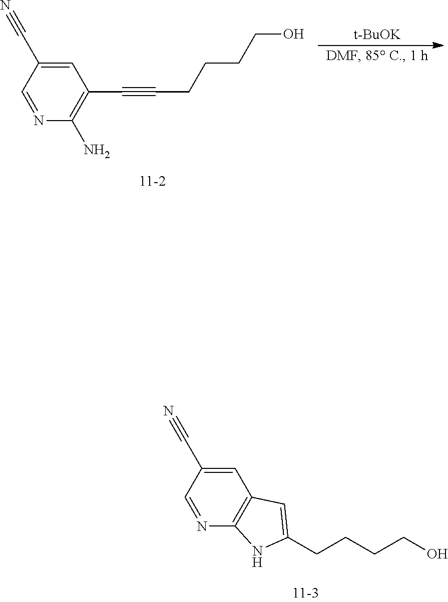 [SMILES: "t-BuOK" ; N#Cc1cnc(N)c(C#CCCCCO)c1 ; "DMF, 85° C., 1 h" ; N#Cc1cnc2[nH]c(CCCCO)cc2c1]

To a stirred solution of 11-2 (28.0 g, 130.1 mmol) in DMF (200 mL) was added t-BuOK (29.2 g, 260.2 mmol). The reaction mixture was stirred at 85° C. for 1 h under nitrogen atmosphere, then H₂O (800 mL) and EA (800 mL) were added and the organic phases was washed with brine, evaporated under reduced pressure. The mixture was purified by silica gel chromatograph column (MeOH/DCM: 0:1 to 1:10) to afford 11-3 (16 g, yield: 57%) as a yellow solid.

### 11.3 The Synthesis of Intermediate 11-4

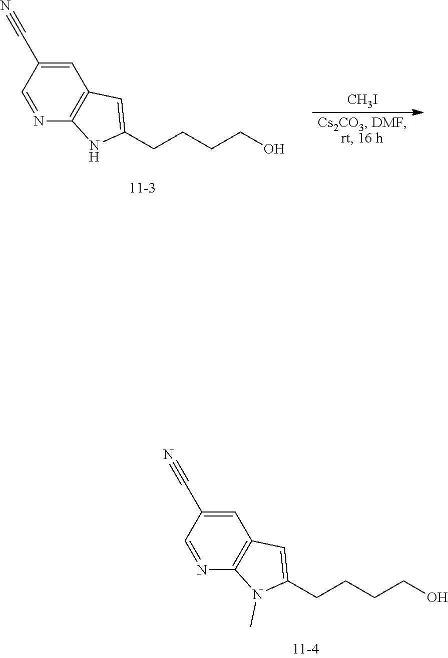 [SMILES: N#Cc1cnc2[nH]c(CCCCO)cc2c1 ; "CH3I" ; "Cs2CO3, DMF,rt, 16 h" ; Cn1c(CCCCO)cc2cc(C#N)cnc21]

To a stirred solution of 11-3 (16 g, 74.4 mmol) in DMF (120 mL) was added Mel (12.6 g, 89.3 mmol) and Cs₂CO₃ (48.5 g, 148.8 mmol). The reaction mixture was stirred at room temperature for 16 h until the reaction was complete (by LCMS), then H₂O (500 mL) and EA (500 mL) were added and the organic phase was collected and evaporated under reduced pressure. The mixture was purified by silica gel chromatograph column (EA/PE: 0:1 to 1:0) to give 11-4 (9.5 g, yield: 56%) as a yellow solid.

### 11.4 The Synthesis of Intermediate 11-5

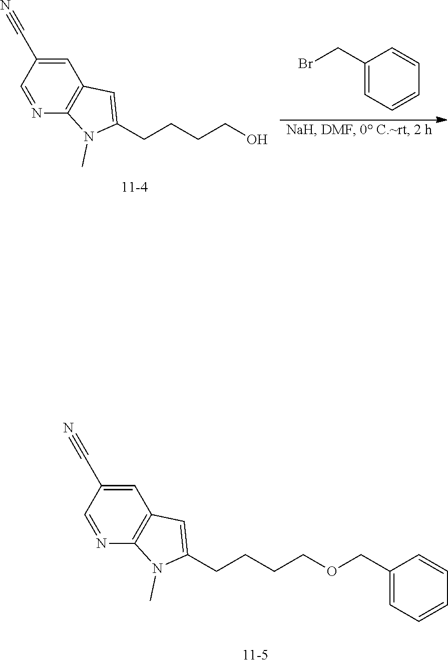

To a stirred solution of 11-4 (12.5 g, 54.6 mmol) and (bromomethyl)benzene (10.3 g, 60.0 mmol) in DMF (100 mL) was added NaH (60% dispersion in mineral oil, 4.4 g, 109.2 mmol) at 0° C. The reaction mixture was stirred at room temperature for 2 hour, then H₂O (400 mL) and EA (350 mL) were added and the organic phase was washed with brine, evaporated under reduced pressure. The mixture was purified by silica gel chromatograph column (EA/PE: 0:1 to 1:2) to give 11-5 (14.5 g, yield: 83%) as a yellow solid.

### 11.5 The Synthesis of Intermediate 11-6

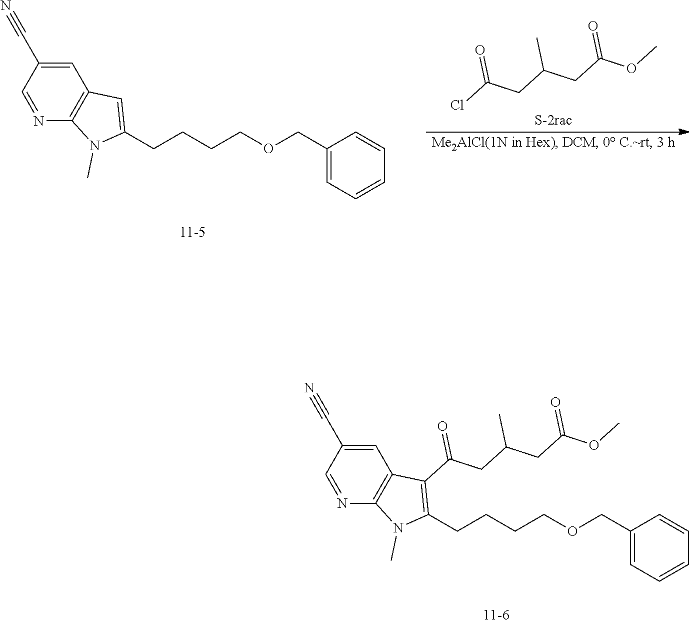

To a stirred solution of 11-5 (14.0 g, 43.8 mmol) in dichloromethane (100 mL) was added Me₂AlCl (1 M in hexane, 87.7 mL, 87.7 mmol) at 0° C. After 5 min, S-2rac (11.7 g, 65.7 mmol) in CH₂Cl₂ (10 mL) was added dropwise and the reaction mixture was stirred at room temperature for another 3 h. The reaction was quenched by adding water and extracted with EtOAc. The organic layer was combined, washed with brine and dried over Na₂SO₄. The solvent was evaporated under reduced pressure and the crude was purified by silica gel chromatography (50% EtOAc/Hex) to afford 11-6 (2.2 g, 11%) as yellow oil.

### 11.6 The Synthesis of Compounds I-105 and I-106

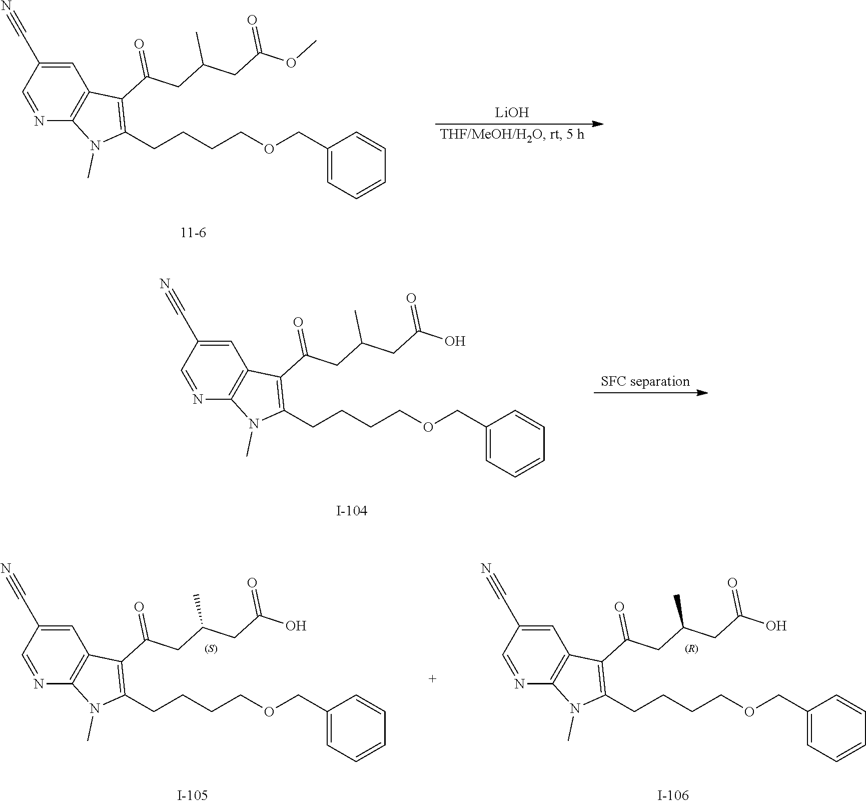

To a mixture of 11-6 (2.2 g, 4.8 mmol) in THF/MeOH/H₂O (20 mL/4 mL/6 mL) was added lithium hydroxide (2.0 g, 47.6 mmol), the reaction mixture was stirred at room temperature for 5 h until the reaction was complete (by LCMS). The mixture was acidified with HCl (1.0 M) to pH ˜5.0 then extracted with EtOAc, the combined organic layer was washed with brine, dried over anhydrous Na₂SO₄ and evaporated under reduced pressure to afford I-104. The crude product was purified by Prep-HPLC then SFC to afford 1-105 and 1-106 (590 mg, 27%) as a white solid.

Additional exemplary compounds were prepared following methods substantially similar to the method described above and herein. Data for these compounds are provided in Tables 12 and 12a below.

TABLE 12
Characterization Data for Additional Exemplary Compounds

| Compound | | | |
|---|---|---|---|
| No. | Chemical Structure | M + 1 | ¹H NMR (400 MHz) |
| I-104 | 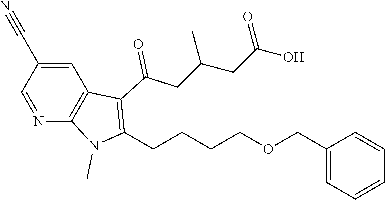 [SMILES: CC(CC(=O)O)CC(=O)c1c(CCCCOCc2ccccc2)n(C)c2ncc(C#N)cc12] | 448.0 | ¹H NMR (400 MHz, CDCl₃) δ 8.57 (d, J = 1.6 Hz, 1H), 8.54 (d, J = 2.0 Hz, 1H), 7.35-7.29 (m, 5H), 4.52 (s, 2H), 3.85 (s, 3H), 3.56 (t, J = 5.2 Hz, 2H), 3.28- 3.24 (m, 2H), 3.09 (dd, J = 16.0, 6.4 Hz, 1H), 2.88 (dd, J = 16.4, 6.8 Hz, 1H), 2.77- 2.72 (m, 1H), 2.49 (dd, J = 15.2, 6.4 Hz, 1H), 2.40 (dd, J = 15.2, 6.4 Hz, 1H), 1.80- 1.78 (m, 4H), 1.14 (d, J = 6.8 Hz, 3H). |
| I-105 | 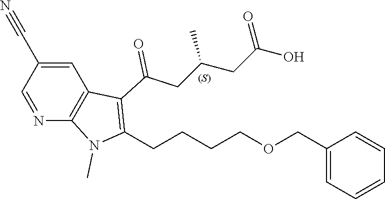 | 448.0 | ¹H NMR (400 MHz, CDCl₃) δ 8.57 (d, J = 1.6 Hz, 1H), 8.54 (d, J = 2.0 Hz, 1H), 7.35-7.29 (m, 5H), 4.52 (s, 2H), 3.85 (s, 3H), 3.56 (t, J = 5.2 Hz, 2H), 3.28- 3.24 (m, 2H), 3.09 (dd, J = 16.0, 6.4 Hz, 1H), 2.88 (dd, J = 16.4, 6.8 Hz, 1H), 2.77- 2.72 (m, 1H), 2.49 (dd, J = 15.2, 6.4 Hz, 1H), 2.40 (dd, J = 15.2, 6.4 Hz, 1H), 1.80- 1.78 (m, 4H), 1.14 (d, J = 6.8 Hz, 3H). |
| I-106 | 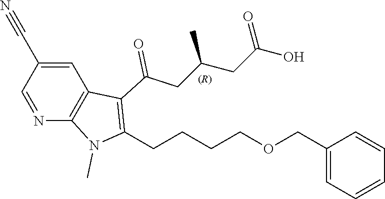 | 448.3 | ¹H NMR (400 MHz, DMSO-d₆) δ 12.09 (s, 1H), 8.83 (d, J = 1.6 Hz, 1H), 8.73 (d, J = 1.6 Hz, 1H), 7.35-7.25 (m, 5H), 4.46 (s, 2H), 3.84 (s, 3H), 3.50 (t, J = 5.2 Hz, 2H), 3.25 (t, J = 6.8 Hz, 2H), 3.10 (dd, J = 16.8, 5.6 Hz, 1H), 2.92 (dd, J = 16.8, 7.6 Hz, 1H), 2.50-2.47 (m, 1H), 2.41 (dd, J = 15.6, 6.0 Hz, 1H), 2.18 (dd, J = 15.2, 7.6 Hz, 1H), 1.70-1.69 (m, 4H), 0.96 (d, J = 6.4 Hz, 3H). |
| I-365 | 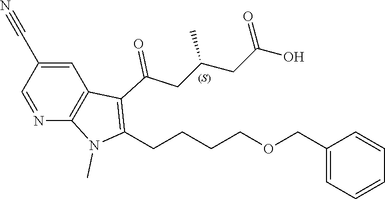 | 448.3 | ¹H NMR (400 MHz, DMSO-d₆) δ 12.09 (br., 1H), 8.83 (d, J = 2.0 Hz, 1H), 8.73 (d, J = 2.0 Hz, 1H), 7.35-7.25 (m, 5H), 4.46 (s, 2H), 3.84 (s, 3H), 3.50 (t, J = 5.2 Hz, 2H), 3.25 (t, J = 6.4 Hz, 2H), 3.10 (dd, J = 16.4, 5.2 Hz, 1H), 2.92 (dd, J = 16.8, 7.6 Hz, 1H), 2.50-2.47 (m, 1H), 2.41 (dd, J = 15.6, 5.6 Hz, 1H), 2.18 (dd, J = 15.2, 7.6 Hz, 1H), 1.70-1.69 (m, 4H), 0.96 (d, J = 6.4 Hz, 3H). |
| I-366 | 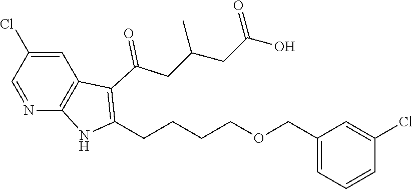 [SMILES: CC(CC(=O)O)CC(=O)c1c(CCCCOCc2cccc(Cl)c2)[nH]c2ncc(Cl)cc12] | 477.2 | ¹H NMR (400 MHz, DMSO-d₆) δ 12.64 (s, 1H), 8.34 (d, J = 2.0 Hz, 1H), 8.25 (d, J = 2.4 Hz, 1H), 7.38-7.31 (m, 3H), 7.27- 7.25 (m, 1H), 4.44 (s, 2H), 3.46 (t, J = 6.0 Hz, 2H), 3.11 (t, J = 7.2 Hz, 2H), 3.00 (dd, J = 16.4 Hz, 5.2 Hz, 1H), 2.83 (dd, J = 16.4 Hz, 7.6 Hz, 1H), 2.47-2.44 (m, 1H), 2.38 (dd, J = 15.2 Hz, 5.6 Hz, 1H), 2.16 (dd, J = 15.2 Hz, 7.6 Hz, 1H), 1.79-1.72 (m, 2H), 1.64-1.57 (m, 2H), 0.95 (d, J = 6.4 Hz, 3H). |
| I-367 | 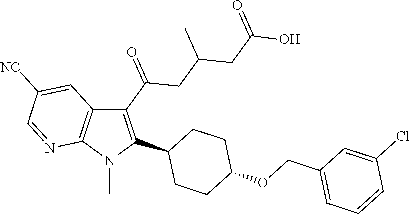 [SMILES: CC(CC(=O)O)CC(=O)c1c2cc(C#N)cnc2n(C)c1[C@H]1CC[C@H](OCc2cccc(Cl)c2)CC1] | 508.2 | ¹H NMR (400 MHz, DMSO-d₆) δ 12.11 (s, 1H), 8.81 (d, J = 2.0 Hz, 1H), 8.73 (d, J = 2.0 Hz, 1H), 7.42-7.31 (m, 4H), 4.59 (s, 2H), 3.96 (s, 3H), 3.58-3.48 (m, 2H), 3.13 (dd, J = 16.8 Hz, 5.6 Hz, 1H), 2.93 (dd, J = 16.8 Hz, 8.0 Hz, 1H), 2.47-2.38 (m, 2H), 2.22-2.12 (m, 5H), 1.78-1.72 (m, 2H), 1.44-1.33 (m, 2H), 0.96 (d, J = 6.4 Hz, 3H). |
| I-368 | 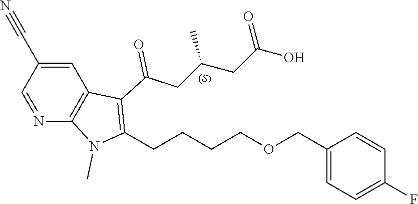 | 466.4 | ¹H NMR (400 MHz, DMSO-d₆) δ 12.10 (s, 1H), 8.84 (d, J = 2.0 Hz, 1H), 8.73 (d, J = 2.0 Hz, 1H), 7.37-7.34 (m, 2H), 7.17- 7.13 (m, 2H), 4.44 (s, 2H), 3.85 (s, 3H), 3.49 (t, J = 5.6 Hz, 2H), 3.24 (t, J = 7.2 Hz, 2H), 3.10 (dd, J = 17.2 Hz, 5.6 Hz, 1H), 2.92 (dd, J = 16.8 Hz, 7.6 Hz, 1H), 2.50-2.48 (m, 1H), 2.41 (dd, J = 15.6 Hz, 5.6 Hz, 1H), 2.18 (dd, J = 15.2 Hz, 8.0 Hz, 1H), 1.74-1.66 (m, 4H), 0.96 (d, J = 6.8 Hz, 3H). |
| I-369 | 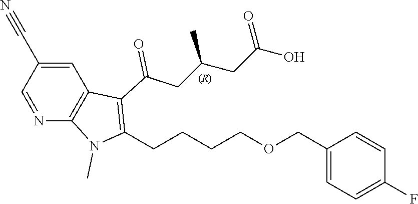 | 466.3 | ¹H NMR (400 MHz, DMSO-d₆) δ 12.11 (s, 1H), 8.84 (s, 1H), 8.74 (s, 1H), 7.37- 7.34 (m, 2H), 7.17-7.13 (m, 2H), 4.44 (s, 2H), 3.85 (s, 3H), 3.53-3.48 (m, 2H), 3.28-3.20 (m, 2H), 3.09 (dd, J = 16.8 Hz, 5.2 Hz, 1H), 2.92 (dd, J = 16.8 Hz, 7.6 Hz, 1H), 2.50-2.48 (m, 1H), 2.41 (dd, J = 15.6 Hz, 5.6 Hz, 1H), 2.18 (dd, J = 15.6 Hz, 8.0 Hz, 1H), 1.76-1.63 (m, 4H), 0.96 (d, J = 6.0 Hz, 3H). |
| I-370 | 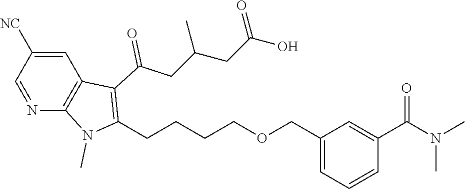 [SMILES: CC(CC(=O)O)CC(=O)c1c(CCCCOCc2cccc(C(=O)N(C)C)c2)n(C)c2ncc(C#N)cc12] | 519.3 | ¹H NMR (400 MHz, DMSO-d₆) δ 12.18 (br., 1H), 8.83 (d, J = 1.6 Hz, 1H), 8.73 (d, J = 1.6 Hz, 1H), 7.42-7.37 (m, 2H), 7.32-7.28 (m, 2H), 4.50 (s, 2H), 3.84 (s, 3H), 3.52 (t, J = 5.6 Hz, 2H), 3.25 (t, J = 7.6 Hz, 2H), 3.10 (dd, J = 16.8, 5.6 Hz, 1H), 2.97-2.88 (m, 7H), 2.49-2.47 (m, 1H), 2.41 (dd, J = 15.2, 5.6 Hz, 1H), 2.18 (dd, J = 15.6, 8.0 Hz, 1H), 1.70-1.67 (m, 4H), 0.96 (d, J = 6.8 Hz, 3H). |
| I-371 | 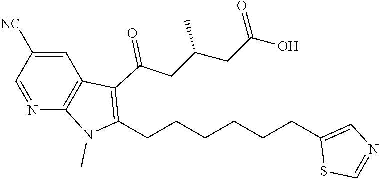 [SMILES: C[C@H](CC(=O)O)CC(=O)c1c(CCCCCCc2cncs2)n(C)c2ncc(C#N)cc12] | 453.3 | ¹H NMR (400 MHz, DMSO-d₆) δ 12.09 (br., 1H), 8.91 (s, 1H), 8.84 (s, 1H), 8.74 (s, 1H), 7.64 (s, 1H), 3.86 (s, 3H), 3.21 (t, J = 7.2 Hz, 2H), 3.10 (dd, J = 16.8, 5.6 Hz, 1H), 2.92 (dd, J = 16.4 Hz, 7.2 Hz, 1H), 2.85 (t, J = 7.6 Hz, 2H), 2.49-2.45 (m, 1H), 2.41 (dd, J = 15.6 Hz, 5.6 Hz, 1H), 2.18 (dd, J = 15.6 Hz, 8.0 Hz, 1H), 1.68-1.56 (m, 4H), 1.50-1.32 (m, 4H), 0.97 (d, J = 6.4 Hz, 3H). |
| I-372 | 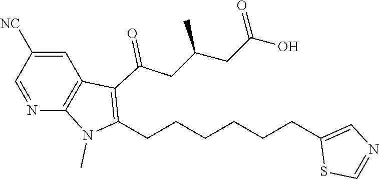 [SMILES: C[C@@H](CC(=O)O)CC(=O)c1c(CCCCCCc2cncs2)n(C)c2ncc(C#N)cc12] | 453.3 | ¹H NMR (400 MHz, DMSO-d₆) δ 12.10 (s, 1H), 8.91 (s, 1H), 8.84 (d, J = 1.6 Hz, 1H), 8.73 (d, J = 2.0 Hz, 1H), 7.64 (s, 1H), 3.86 (s, 3H), 3.21 (t, J = 7.6 Hz, 2H), 3.10 (dd, J = 16.8 Hz, 5.6 Hz, 1H), 2.92 (dd, J = 16.8 Hz, 7.6 Hz, 1H), 2.85 (t, J = 7.2 Hz, 2H), 2.49-2.45 (m, 1H), 2.41 (dd, J = 15.6 Hz, 5.6 Hz, 1H), 2.18 (dd, J = 15.2 Hz, 7.6 Hz, 1H), 1.65-1.58 (m, 4H), 1.48- 1.36 (m, 4H), 0.97 (d, J = 6.4 Hz, 3H). |
| I-373 | 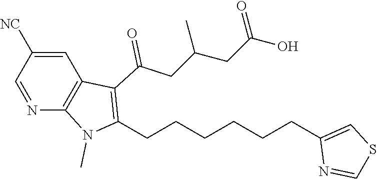 [SMILES: CC(CC(=O)O)CC(=O)c1c(CCCCCCc2cscn2)n(C)c2ncc(C#N)cc12] | 453.2 | ¹H NMR (400 MHz, DMSO-d₆) δ 8.99 (d, J = 2.0 Hz, 1H), 8.83 (d, J = 2.0 Hz, 1H), 8.73 (d, J = 1.6 Hz, 1H), 7.31-7.30 (m, 1H), 3.86 (s, 3H), 3.21 (t, J = 7.6 Hz, 2H), 3.11 (dd, J = 5.6, 16.8 Hz, 1H), 2.91 (dd, J = 7.6, 16.8 Hz, 1H), 2.75 (t, J = 14.8 Hz, 2H), 2.55-2.45 (m, 1H), 2.41 (dd, J = 6.0, 15.2 Hz, 1H), 2.18 (dd, J = 8.0, 15.6 Hz, 1H), 1.72-1.56 (m, 4H), 1.50-1.35 (m, 4H), 0.96 (d, J = 6.4 Hz, 3H). |
| I-374 | 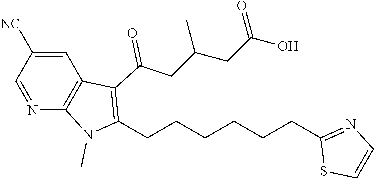 [SMILES: CC(CC(=O)O)CC(=O)c1c(CCCCCCc2nccs2)n(C)c2ncc(C#N)cc12] | 453.2 | ¹H NMR (400 MHz, DMSO-d₆) δ 12.11 (s, 1H), 8.81 (d, J = 6.4 Hz, 1H), 8.71 (d, J = 6.8 Hz, 1H), 7.67-7.66 (m, 1H), 7.55- 7.54 (m, 1H), 3.85 (s, 3 H), 3.21-3.19 (m, 2H), 3.10 (dd, J = 16.8 Hz, J = 5.2 Hz, 1H), 3.00-2.89 (m, 3 H), 2.52-2.46 (m, 1 H), 2.42 (dd, J = 15.6 Hz, J = 5.6 Hz, 1H), 2.19 (dd, J = 15.6 Hz, J = 8.0 Hz, 1H), 175-1.72 (m, 2H), 1.60 (m, 2H), 1.46- 1.41 (m, 4H), 0.97 (d, J = 6.4 Hz, 3H). |
| I-375 | 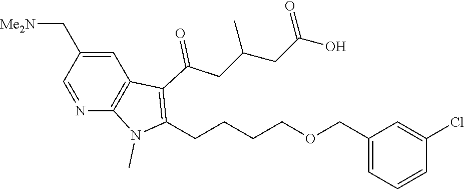 [SMILES: CC(CC(=O)O)CC(=O)c1c(CCCCOCc2cccc(Cl)c2)n(C)c2ncc(CN(C)C)cc12] | 514.3 | ¹H NMR (400 MHz, DMSO-d₆) δ 8.23- 8.22 (m, 2H), 7.39-7.32 (m, 3H), 7.28 (d, J = 7.2 Hz, 1H), 4.47 (s, 2H), 3.80 (s, 3 H), 3.66-3.58 (m, 4H), 3.26-3.18 (m, 2H), 3.07 (dd, J = 15.6 Hz, J = 5.2 Hz, 1H), 2.75 (dd, J = 15.6 Hz, J = 8.4 Hz, 1H), 2.46-2.41 (m, 1H), 2.33 (dd, J = 15.2 Hz, J = 6.0 Hz, 1H), 2.21 (s, 6H), 2.16 (dd, J = 15.2 Hz, J = 7.2 Hz, 1H), 1.69-1.66 (m, 4H), 0.95 (d, J = 6.4 Hz, 3H). |
| I-376 | 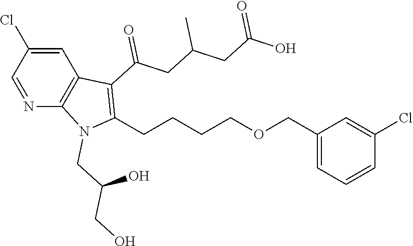 [SMILES: CC(CC(=O)O)CC(=O)c1c(CCCCOCc2cccc(Cl)c2)n(C[C@@H](O)CO)c2ncc(Cl)cc12] | 551.2 | ¹H NMR (400 MHz, DMSO-d₆) δ 12.07 (s, 1H), 8.37 (d, J = 2.0 Hz, 1H), 8.32 (d, J = 2.4 Hz, 1H), 7.39-7.27 (m, 4H), 5.00 (s, 1H), 4.84 (s, 1H), 4.50-4.46 (m, 3H), 4.14-4.09 (m, 1H), 3.91 (s, 1H), 3.49 (t, J = 5.6 Hz, 2H), 3.41 (s, 2H), 3.32 (s, 1H), 3.28-3.22 (m, 1H), 3.09-3.01 (m, 1H), 2.92-2.83 (m, 1H), 2.47 (s, 1H), 2.43-2.37 (m, 1H), 2.21-2.14 (m, 1H), 1.69-1.60 (m, 4H), 0.98-0.95 (m, 3H). |
| I-377 | 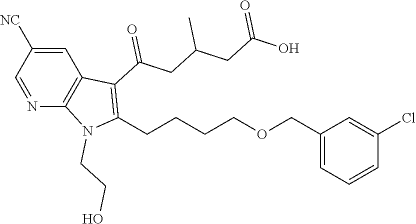 [SMILES: CC(CC(=O)O)CC(=O)c1c(CCCCOCc2cccc(Cl)c2)n(CCO)c2ncc(C#N)cc12] | 512.2 | ¹H NMR (400 MHz, CDCl₃) δ 8.54 (d, J = 1.6 Hz, 1H), 8.51 (d, J = 2.0 Hz, 1H), 7.32 (s, 1H), 7.27-7.18 (m, 3H), 4.52-4.45 (m, 4H), 3.99 (t, J = 4.8 Hz, 2H), 3.56 (t, J = 5.2 Hz, 2H), 3.32-3.29 (m, 2H), 3.10 (dd, J = 16.0 Hz, 6.4 Hz, 1H), 2.86 (dd, J = 16.0 Hz, 5.6 Hz, 1H), 2.77-2.67 (m, 1H), 2.48 (dd, J = 15.2 Hz, 6.8 Hz, 1H), 2.40 (dd, J = 15.6 Hz, 5.6 Hz, 1H), 1.85-1.72 (m, 4H), 1.12 (d, J = 6.8 Hz, 3H). |
| I-378 | 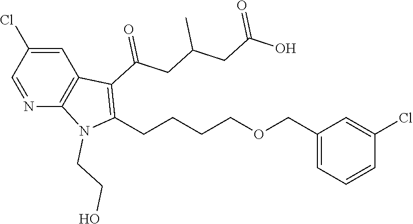 [SMILES: CC(CC(=O)O)CC(=O)c1c(CCCCOCc2cccc(Cl)c2)n(CCO)c2ncc(Cl)cc12] | 521.2 | ¹H NMR (400 MHz, DMSO-d₆) δ 12.08 (s, 1H), 8.37 (d, J = 2.0 Hz, 1H), 8.32 (d, J = 2.0 Hz, 1H), 7.39-7.28 (m, 4H), 4.98 (s, 1H), 4.48 (s, 2H), 4.35 (t, J = 5.6 Hz, 2H), 3.73 (d, J = 4.4 Hz, 2H), 3.51 (t, J = 5.2 Hz, 2H), 3.26 (t, J = 7.6 Hz, 2H), 3.05 (dd, J = 16.8, 5.6 Hz, 1H), 2.88 (dd, J = 16.8, 7.6 Hz, 1H), 2.51-2.48 (m, 1H), 2.41 (dd, J = 15.6, 6.0 Hz, 1H), 2.18 (dd, J = 15.6, 8.0 Hz, 1H), 1.78-1.63 (m, 4H), 0.97 (d, J = 6.4 Hz, 3H). |
| I-379 | 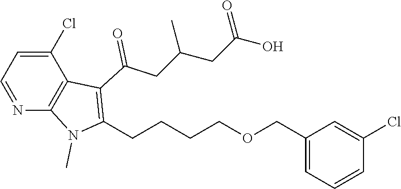 [SMILES: CC(CC(=O)O)CC(=O)c1c(CCCCOCc2cccc(Cl)c2)n(C)c2nccc(Cl)c12] | 491.2 | ¹H NMR (400 MHz, DMSO-d₆) δ 12.07 (s, 1H), 8.23 (d, J = 4.8 Hz, 1H), 7.39-7.28 (m, 5H), 4.47 (s, 2H), 3.80 (s, 3H), 3.51- 3.47 (m, 2H), 2.99 (dd, J = 16.4, 5.6 Hz, 1H), 2.93-2.89 (m, 2H), 2.82 (dd, J = 16.0, 7.6 Hz, 1H), 2.45-2.38 (m, 1H), 2.31 (dd, J = 15.2, 5.2 Hz, 1H), 2.11 (dd, J = 15.2, 8.0 Hz, 1H), 1.75-1.64 (m, 4H), 0.92 (d, J = 6.4 Hz, 3H). |
| I-380 | 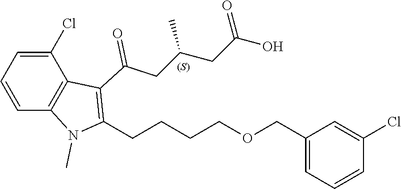 | 490.2 | ¹H NMR (400 MHz, DMSO-d₆) δ 12.06 (br., 1H), 7.51 (dd, J = 7.2, 2.0 Hz, 1H), 7.39-7.32 (m, 3H), 7.29-7.27 (m, 1H), 7.22-7.16 (m, 2H), 4.47 (s, 2H), 3.74 (s, 3H), 3.48 (t, J = 5.6 Hz, 2H), 2.93 (dd, J = 16.4, 5.6 Hz, 1H), 2.83-2.77 (m, 3H), 2.40-2.36 (m, 1H), 2.32 (dd, J = 15.2, 5.6 Hz, 1H), 2.09 (dd, J = 15.2, 8.0 Hz, 1H), 1.68-1.65 (m, 4H), 0.92 (d, J = 6.8 Hz, 3H). |
| I-381 | 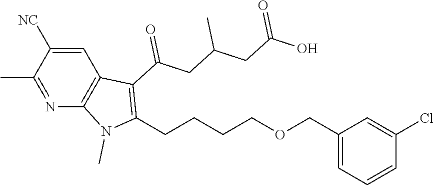 [SMILES: Cc1nc2c(cc1C#N)c(C(=O)CC(C)CC(=O)O)c(CCCCOCc1cccc(Cl)c1)n2C] | 496.2 | ¹H NMR (400 MHz, DMSO-d₆) δ 12.11 (br., 1H), 8.71-8.70 (m, 1H), 7.38-7.31 (m, 3H), 7.29-7.27 (m, 1H), 4.47 (s, 2H), 3.80 (s, 3H), 3.50 (t, J = 5.6 Hz, 2H), 3.23- 3.22 (m, 2H), 3.06 (dd, J = 16.4, 5.2 Hz, 1H), 2.89 (dd, J = 12.8, 7.6 Hz, 1H), 2.74 (s, 3H), 2.49-2.39 (m, 2H), 2.18 (dd, J = 15.2, 8.0 Hz, 1H), 1.70-1.66 (m, 4H), 0.96 (d, J = 6.8 Hz, 3H). |
| I-382 | 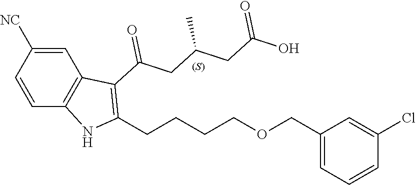 | 467.2 | ¹H NMR (400 MHz, DMSO-d₆) δ 12.36 (s, 1H), 8.40 (s, 1H), 7.56-7.49 (m, 2H), 7.35-7.23 (m, 4H), 4.42 (s, 2H), 3.45 (t, J = 6.0 Hz, 2H), 3.11 (t, J = 7.6 Hz, 2H), 3.02 (dd, J = 16.4 Hz, 5.6 Hz, 1H), 2.86 (dd, J = 16.8 Hz, 7.6 Hz, 1H), 2.57-2.52 (m, 1H), 2.40 (dd, J = 15.6 Hz, 5.6 Hz, 1H), 2.17 (dd, J = 15.6 Hz, 8.0 Hz, 1H), 1.81-1.73 (m, 2H), 1.66-1.55 (m, 2H), 0.97 (d, J = 6.4 Hz, 3H) |
| I-383 | 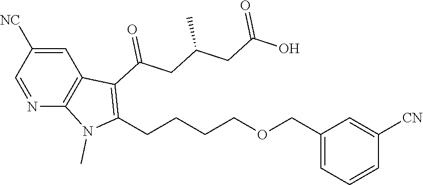 [SMILES: C[C@H](CC(=O)O)CC(=O)c1c(CCCCOCc2cccc(C#N)c2)n(C)c2ncc(C#N)cc12] | 473.2 | ¹H NMR (400 MHz, DMSO-d₆) δ 12.10 (s, 1H), 8.84 (d, J = 1.6 Hz, 1H), 8.74 (d, J = 1.6 Hz, 1H), 7.76-7.74 (m, 2H), 7.67 (d, J = 7.6 Hz, 1H), 7.56 (t, J = 7.6 Hz, 1H), 4.52 (s, 2H), 3.85 (s, 3H), 3.53 (t, J = 5.6 Hz, 2H), 3.25 (t, J = 7.2 Hz, 2H), 3.10 (dd, J = 16.8 Hz, 5.6 Hz, 1H), 2.92 (dd, J = 16.8 Hz, 7.6 Hz, 1H), 2.49-2.46 (m, 1H), 2.41 (dd, J = 15.6 Hz, 5.6 Hz, 1H), 2.18 (dd, J = 15.6 Hz, 8.0 Hz, 1H), 1.75-1.63 (m, 4H), 0.96 (d, J = 6.8 Hz, 3H). |
| I-384 | 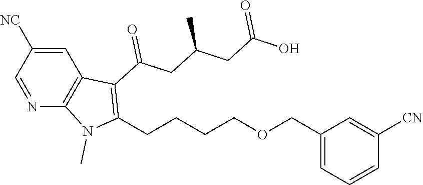 [SMILES: C[C@@H](CC(=O)O)CC(=O)c1c(CCCCOCc2cccc(C#N)c2)n(C)c2ncc(C#N)cc12] | 473.3 | ¹H NMR (400 MHz, DMSO-d₆) δ 12.10 (s, 1H), 8.84 (d, J = 2.0 Hz, 1H), 8.74 (d, J = 1.6 Hz, 1H), 7.76-7.74 (m, 2H), 7.67 (d, J = 7.6 Hz, 1H), 7.56 (t, J = 8.0 Hz, 1H), 4.52 (s, 2H), 3.85 (s, 3H), 3.53 (t, J = 5.6 Hz, 2H), 3.25 (t, J = 7.2 Hz, 2H), 3.10 (dd, J = 16.8 Hz, 5.6 Hz, 1H), 2.92 (dd, J = 16.8 Hz, 7.6 Hz, 1H), 2.49-2.46 (m, 1H), 2.41 (dd, J = 15.6 Hz, 5.6 Hz, 1H), 2.18 (dd, J = 15.6 Hz, 8.0 Hz, 1H), 1.75-1.63 (m, 4H), 0.96 (d, J = 6.4 Hz, 3H). |
| I-385 | 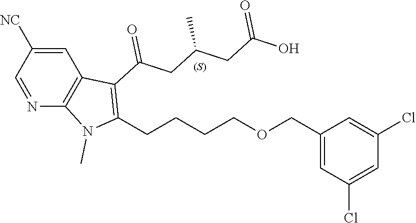 | 516.2 | ¹H NMR (400 MHz, DMSO-d₆) δ 12.10 (s, 1H), 8.84 (d, J = 1.6 Hz, 1H), 8.74 (d, J = 2.0 Hz, 1H), 7.51 (t, J = 1.6 Hz, 1H), 7.36 (d, J = 2.0 Hz, 2H), 4.48 (s, 2H), 3.86 (s, 3H), 3.51 (t, J = 5.2 Hz, 2H), 3.25 (t, J = 7.2 Hz, 2H), 3.10 (dd, J = 16.8 Hz, 5.6 Hz, 1H), 2.92 (dd, J = 16.8 Hz, 7.6 Hz, 1H), 2.51 (s, 1H), 2.41 (dd, J = 15.6 Hz, 5.6 Hz, 1H), 2.18 (dd, J = 15.2 Hz, 8.0 Hz, 1H), 1.70-1.69 (m, 4H), 0.96 (d, J = 6.8 Hz, 3H). |
| I-386 | 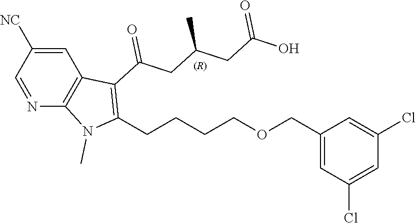 | 516.3 | ¹H NMR (400 MHz, DMSO-d₆) δ 12.11 (s, 1H), 8.84 (d, J = 2.0 Hz, 1H), 8.74 (d, J = 2.0 Hz, 1H), 7.51 (t, J = 1.6 Hz, 1H), 7.36 (d, J = 1.6 Hz, 2H), 4.48 (s, 2H), 3.86 (s, 3H), 3.51 (t, J = 5.6 Hz, 2H), 3.25 (t, J = 7.2 Hz, 2H), 3.10 (dd, J = 16.8 Hz, 5.2 Hz, 1H), 2.92 (dd, J = 16.8 Hz, 7.6 Hz, 1H), 2.51 (s, 1H), 2.41 (dd, J = 15.6 Hz, 6.0 Hz, 1H), 2.18 (dd, J = 15.2 Hz, 7.6 Hz, 1H), 1.72-1.67 (m, 4H), 0.96 (d, J = 6.4 Hz, 3H). |
| I-387 | 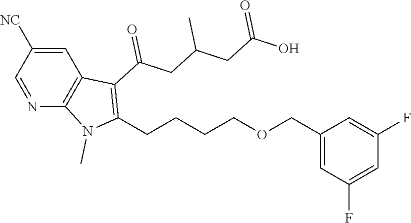 [SMILES: CC(CC(=O)O)CC(=O)c1c(CCCCOCc2cc(F)cc(F)c2)n(C)c2ncc(C#N)cc12] | 484.3 | ¹H NMR (400 MHz, DMSO-d₆) δ 12.16 (br., 1H), 8.82 (s, 1H), 8.72 (s, 1H), 7.11 (t, J = 9.2 Hz, 1H), 7.03 (d, J = 6.8 Hz, 1H), 4.50 (s, 2H), 3.86 (s, 3H), 3.53 (t, J = 5.6 Hz, 2H), 3.26 (t, J = 7.2 Hz, 2H), 3.11 (dd, J = 16.8 Hz, J = 5.2 Hz, 1H), 2.92 (dd, J = 16.8 Hz, J = 7.6 Hz, 1H), 2.52-2.49 (m, 1H), 2.43 (dd, J = 15.6 Hz, J = 5.6 Hz, 1H), 2.20 (dd, J = 15.6 Hz, J = 8.0 Hz, 1H), 1.72-1.68 (m, 4H), 0.98 (d, J = 6.8 Hz, 3H). |
| I-388 | 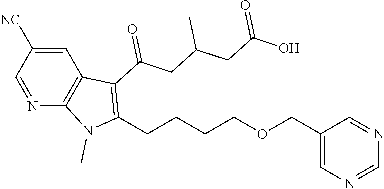 [SMILES: CC(CC(=O)O)CC(=O)c1c(CCCCOCc2cncnc2)n(C)c2ncc(C#N)cc12] | 450.3 | ¹H NMR (400 MHz, DMSO-d₆) δ 12.3 (br., 1H), 9.12 (s, 1H), 8.85 (d, J = 1.6 Hz, 1H), 8.77 (s, 2H), 8.74 (d, J = 1.6 Hz, 1H), 4.53 (s, 2H), 3.85 (s, 3H), 3.55 (t, J = 5.6 Hz, 2H), 3.25 (t, J = 6.4 Hz, 2H), 3.11 (dd, J = 16.8 Hz, 4.8 Hz, 1H), 2.90 (dd, J = 16.8 Hz, 7.6 Hz, 1H), 2.44- 2.31 (m, 2H), 2.16 (dd, J = 15.2 Hz, 8.0 Hz, 1H), 1.75-1.62 (m, 4H), 0.95 (d, J = 6.4 Hz, 3H). |
| I-389 | 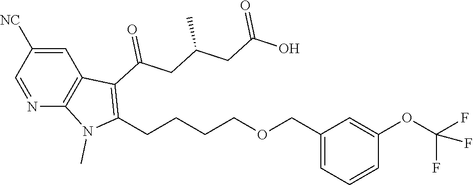 [SMILES: C[C@H](CC(=O)O)CC(=O)c1c(CCCCOCc2cccc(OC(F)(F)F)c2)n(C)c2ncc(C#N)cc12] | 532.2 | ¹H NMR (400 MHz, DMSO-d₆) δ 12.21 (br., 1H), 8.84 (d, J = 1.6 Hz, 1H), 8.73 (d, J = 1.6 Hz, 1H), 7.47 (t, J = 8.0 Hz, 1H), 7.35 (d, J = 7.6 Hz, 1H), 7.28-7.25 (m, 2H), 4.52 (s, 2H), 3.84 (s, 3H), 3.52 (t, J = 5.6 Hz, 2H), 3.27-3.23 (m, 2H), 3.11 (dd, J = 16.8 Hz, 5.6 Hz, 1H), 2.88 (dd, J = 16.8 Hz, 8.0 Hz, 1H), 2.50-2.46 (m, 1H), 2.42 (dd, J = 15.6 Hz, 6.0 Hz, 1H), 2.18 (dd, J = 15.6 Hz, 8.0 Hz, 1H), 1.76-1.60 (m, 4H), 0.95 (d, J = 6.8 Hz, 3H). |
| I-390 | 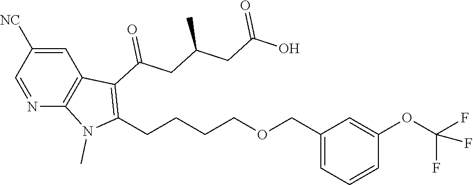 [SMILES: C[C@@H](CC(=O)O)CC(=O)c1c(CCCCOCc2cccc(OC(F)(F)F)c2)n(C)c2ncc(C#N)cc12] | 532.2 | ¹H NMR (400 MHz, DMSO-d₆) δ 12.11 (br., 1H), 8.84 (d, J = 1.2 Hz, 1H), 8.73 (d, J = 1.6 Hz, 1H), 7.47 (t, J = 8.0 Hz, 1H), 7.35 (d, J = 7.6 Hz, 1H), 7.28-7.25 (m, 2H), 4.52 (s, 2H), 3.84 (s, 3H), 3.52 (t, J = 5.6 Hz, 2H), 3.25 (t, J = 6.8 Hz, 2H), 3.11 (dd, J = 16.8 Hz, 5.6 Hz, 1H), 2.88 (dd, J = 16.8 Hz, 8.0 Hz, 1H), 2.50- 2.46 (m, 1H), 2.42 (dd, J = 15.6 Hz, 6.0 Hz, 1H), 2.15 (dd, J = 15.2 Hz, 8.0 Hz, 1H), 1.76-1.60 (m, 4H), 0.95 (d, J = 6.4 Hz, 3H). |
| I-391 | 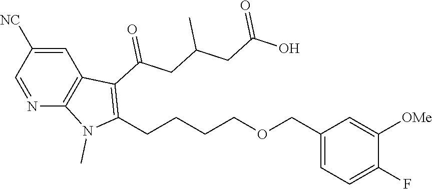 [SMILES: COc1cc(COCCCCc2c(C(=O)CC(C)CC(=O)O)c3cc(C#N)cnc3n2C)ccc1F] | 496.3 | ¹H NMR (400 MHz, DMSO-d₆) δ 8.40 (d, J = 2.0 Hz, 1H), 8.73 (d, J = 1.6 Hz, 1H), 7.15 (dd, J = 12.0 Hz, 8.4 Hz, 1H), 7.08 (dd, J = 8.4 Hz, 1.6 Hz, 1H), 6.89-6.85 (m, 1H), 4.42 (s, 2H), 3.85 (s, 3H), 3.80 (s, 3H), 3.49 (t, J = 5.6 Hz, 2H), 3.26 (t, J = 7.2 Hz, 2H), 3.10 (dd, J = 16.8, 5.6 Hz, 1H), 2.91 (dd, J = 16.8, 7.6 Hz, 1H), 2.50- 2.37 (m, 2H), 2.17 (dd, J = 15.6, 8.0 Hz, 1H), 1.70-1.68 (m, 4H), 0.96 (d, J = 6.4 Hz, 3H). |
| I-392 | 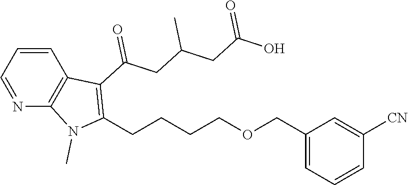 [SMILES: CC(CC(=O)O)CC(=O)c1c(CCCCOCc2cccc(C#N)c2)n(C)c2ncccc12] | 448.3 | ¹H NMR (400 MHz, DMSO-d₆) δ 11.74 (br., 1H), 8.32-8.28 (m, 2H), 7.82-7.74 (m, 2H), 7.68-7.64 (m, 1H), 7.56 (t, J = 7.6 Hz, 1H), 7.28 (dd, J = 4.8 Hz, 2.8 Hz, 1H), 4.52 (s, 2H), 3.82 (s, 3H), 3.53 (t, J = 6.0 Hz, 2H), 3.24 (t, J = 7.2 Hz, 2H), 3.03 (dd, J = 16.4 Hz, 6.0 Hz, 1H), 2.87 (dd, J = 16.8 Hz, 8.0 Hz, 1H), 2.51-2.48 (m, 1H), 2.40 (dd, J = 15.2 Hz, 5.6 Hz, 1H), 2.16 (dd, J = 15.2 Hz, 4.0 Hz, 1H), 1.70-1.67 (m, 4H), 0.97 (d, J = 6.4 Hz, 3H). |
| I-393 | 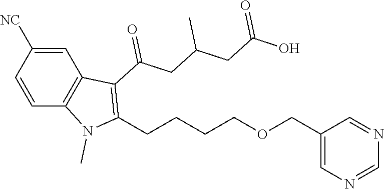 [SMILES: CC(CC(=O)O)CC(=O)c1c(CCCCOCc2cncnc2)n(C)c2ccc(C#N)cc12] | 449.2 | ¹H NMR (400 MHz, DMSO-d₆) δ 12.08 (br., 1H), 9.13 (s, 1H), 8.78 (s, 2H), 8.43 (s, 1H), 7.77 (d, J = 8.4 Hz, 1H), 7.62 (dd, J = 8.4 Hz, 1.2 Hz, 1H), 4.54 (s, 2H), 3.81 (s, 3H), 3.55 (t, J = 6.0 Hz, 2H), 3.22-3.18 (m, 2H), 3.08 (dd, J = 16.8 Hz, 6.0 Hz, 1H), 2.91 (dd, J = 16.8 Hz, 7.6 Hz, 1H), 2.40-2.38 (m, 1H), 2.41 (dd, J = 15.6 Hz, 5.6 Hz, 1H), 2.18 (dd, J = 15.2 Hz, 8.0 Hz, 1H), 1.73-1.65 (m, 4H) , 0.96 (d, J = 6.8 Hz, 3H). |
| I-394 | 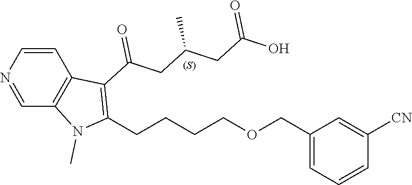 | 448.1 | ¹H NMR (400 MHz, DMSO-d₆) δ 12.15 (br., 1H), 8.91 (s, 1H), 8.30 (d, J = 5.6 Hz, 1H), 7.83 (d, J = 6.0 Hz, 1H), 7.76-7.72 (m, 2H), 7.67 (d, J = 8.0 Hz, 1H), 7.56 (t, J = 8.0 Hz, 1H), 4.52 (s, 2H), 3.86 (s, 3H), 3.52 (t, J = 6.0 Hz, 2H), 3.23 (t, J = 6.0 Hz, 2H), 3.03 (dd, J = 16.0 Hz, 5.6 Hz, 1H), 2.86 (dd, J = 16.0 Hz, 7.6 Hz, 1H), 2.49-2.43 (m, 1H), 2.38 (dd, J = 15.2 Hz, 5.6 Hz, 1H), 2.15 (dd, J = 15.2 Hz, 8.0 Hz, 1H), 1.74-1.60 (m, 4H), 0.96 (d, J = 6.4 Hz, 3H). |
| I-395 | 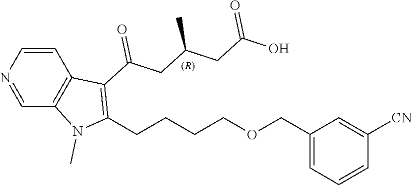 | 448.2 | ¹H NMR (400 MHz, DMSO-d₆) δ 12.08 (s, 1H), 9.06 (s, 1H), 8.36 (d, J = 5.6 Hz, 1H), 7.99 (d, J = 5.6 Hz, 1H), 7.77-7.73 (m, 2H), 7.67 (d, J = 7.6 Hz, 1H), 7.56 (t, J = 8.0 Hz, 1H), 4.52 (s, 2H), 3.90 (s, 3H), 3.52 (t, J = 6.0 Hz, 2H), 3.25 (t, J = 8.0 Hz, 2H), 3.06 (dd, J = 16.4 Hz, 6.0 Hz, 1H), 2.90 (dd, J = 16.4 Hz, 7.6 Hz, 1H), 2.48-2.43 (m, 1H), 2.40 (dd, J = 15.2 Hz, 5.6 Hz, 1H), 2.17 (dd, J = 15.6 Hz, 8.0 Hz, 1H), 1.74-1.63 (m, 4H), 0.97 (d, J = 6.4 Hz, 3H). |
| I-396 | 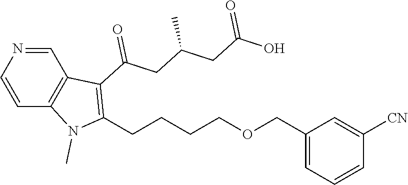 [SMILES: C[C@H](CC(=O)O)CC(=O)c1c(CCCCOCc2cccc(C#N)c2)n(C)c2ccncc12] | 448.3 | ¹H NMR (400 MHz, DMSO-d₆) δ 12.03 (br., 1H), 9.19 (s, 1H), 8.32 (d, J = 6.0 Hz, 1H), 7.75 (d, J = 7.6 Hz, 2H), 7.67 (d, J = 8.0 Hz, 1H), 7.60 (d, J = 5.6 Hz, 1H), 7.56 (t, J = 7.6 Hz, 1H), 4.52 (s, 2H), 3.78 (s, 3H), 3.52 (t, J = 6.0 Hz, 2H), 3.21 (t, J = 7.2 Hz, 2H), 3.08 (dd, J = 16.4 Hz, 6.0 Hz, 1H), 2.94 (dd, J = 16.4 Hz, 7.6 Hz, 1H), 2.51-2.50 (m, 1H), 2.41 (dd, J = 15.2 Hz, 5.6 Hz, 1H), 2.17 (dd, J = 15.6 Hz, 7.6 Hz, 1H), 1.72-1.64 (m, 4H), 0.97 (d, J = 6.4 Hz, 3H). |
| I-397 | 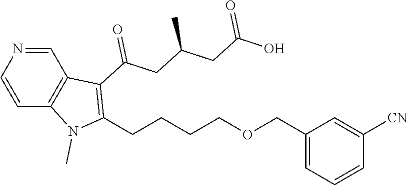 [SMILES: C[C@@H](CC(=O)O)CC(=O)c1c(CCCCOCc2cccc(C#N)c2)n(C)c2ccncc12] | 448.3 | ¹H NMR (400 MHz, DMSO-d₆) δ 12.03 (br., 1H), 9.18 (s, 1H), 8.32 (d, J = 5.6 Hz, 1H), 7.75 (d, J = 7.6 Hz, 2H), 7.67 (d, J = 7.6 Hz, 1H), 7.60 (d, J = 5.6 Hz, 1H), 7.56 (t, J = 8.0 Hz, 1H), 4.52 (s, 2H), 3.77 (s, 3H), 3.52 (t, J = 6.0 Hz, 2H), 3.21 (t, J = 7.6 Hz, 2H), 3.08 (dd, J = 16.4 Hz, 6.0 Hz, 1H), 2.94 (dd, J = 16.4 Hz, 7.6 Hz, 1H), 2.51-2.50 (m, 1H), 2.41 (dd, J = 15.2 Hz, 5.6 Hz, 1H), 2.17 (dd, J = 15.2 Hz, 7.6 Hz, 1H), 1.72-1.64 (m, 4H), 0.97 (d, J = 6.4 Hz, 3H). |
| I-398 | 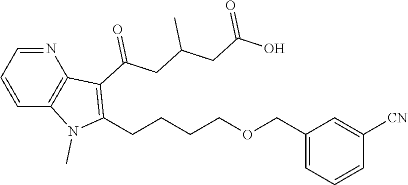 [SMILES: CC(CC(=O)O)CC(=O)c1c(CCCCOCc2cccc(C#N)c2)n(C)c2cccnc12] | 448.3 | ¹H NMR (400 MHz, DMSO-d₆ + D₂O) δ 8.49 (dd, J = 5.6 Hz, 4.8 Hz, 1H), 7.96 (dd, J = 8.4 Hz, 1.2 Hz, 1H), 7.75-7.73 (m, 2H), 7.67 (d, J = 7.6 Hz, 1H), 7.58-7.54 (m, 1H), 7.24 (dd, J = 8.4 Hz, 4.8 Hz, 1H), 4.52 (s, 2H), 3.77 (s, 3H), 3.52 (t, J = 5.6 Hz, 2H), 3.37 (dd, J = 15.2 Hz, 7.6 Hz, 1H), 3.27-3.20 (m, 3H), 2.52-2.42 (m, 1H), 2.26 (dd, J = 14.8, 5.6 Hz, 1H), 2.02 (dd, J = 14.8, 8.8 Hz, 1H), 1.70-1.64 (m, 4H), 0.92 (d, J = 6.8 Hz, 3H). |
| I-399 | 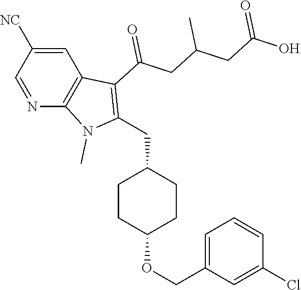 [SMILES: CC(CC(=O)O)CC(=O)c1c(C[C@H]2CC[C@@H](OCc3cccc(Cl)c3)CC2)n(C)c2ncc(C#N)cc12] | 522.3 | ¹H NMR (400 MHz, DMSO-d₆) δ 12.13 (br., 1H), 8.83 (d, J = 1.6 Hz, 1H), 8.74 (d, J = 1.6 Hz, 1H), 7.41-7.37 (m, 2H), 7.35-7.31 (m, 2H), 4.47 (s, 2H), 3.57 (s, 1H), 3.33 (s, 3H), 3.22 (d, J = 7.2 Hz, 2H), 3.11 (dd, J = 16.8 Hz, 5.2 Hz, 1H), 2.94 (dd, J = 16.8 Hz, 8.0 Hz, 1H), 2.50-2.48 (m, 1H), 2.41 (dd, J = 15.6 Hz, 5.6 Hz, 1H), 2.18 (dd, J = 15.6 Hz, 8.0 Hz, 1H), 1.85 (d, J = 10.8 Hz, 2H), 1.80-1.75 (m, 1H), 1.54-1.45 (m, 2H), 1.40-1.34 (m, 4H), 0.97 (d, J = 6.8 Hz, 3H). |
| I-400 | 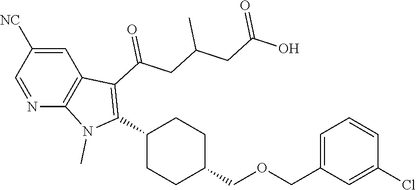 [SMILES: CC(CC(=O)O)CC(=O)c1c2cc(C#N)cnc2n(C)c1[C@H]1CC[C@@H](COCc2cccc(Cl)c2)CC1] | 522.3 | ¹H NMR (400 MHz, DMSO-d₆) δ 12.15 (br., 1H), 8.81 (d, J = 1.6 Hz, 1H), 8.74 (d, J = 1.6 Hz, 1H), 7.42-7.33 (m, 4H), 4.56 (s, 2H), 3.90 (s, 3H), 3.71 (d, J = 7.6 Hz, 2H), 3.61-3.55 (m, 1H), 3.13 (dd, J = 16.8 Hz, 5.6 Hz, 1H), 2.91 (dd, J = 16.8 Hz, 8.0 Hz, 1H), 2.47-2.38 (m, 2H), 2.21- 2.11 (m, 4H), 1.84 (d, J = 12.8 Hz, 2H), 1.64-1.57 (m, 2H), 1.49-1.46 (m, 2H), 0.95 (d, J = 6.8 Hz, 3H). |
| I-401 | 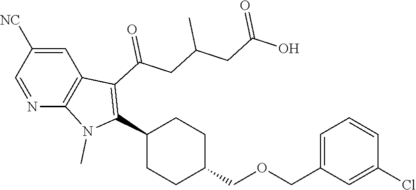 [SMILES: CC(CC(=O)O)CC(=O)c1c2cc(C#N)cnc2n(C)c1[C@H]1CC[C@H](COCc2cccc(Cl)c2)CC1] | 522.3 | ¹H NMR (400 MHz, DMSO-d₆) δ 12.14 (br., 1H), 8.81 (d, J = 1.6 Hz, 1H), 8.74 (d, J = 1.6 Hz, 1H), 7.42-7.30 (m, 4H), 4.50 (s, 2H), 3.97 (s, 3H), 3.68-3.63 (m, 1H), 3.33 (d, J = 6.0 Hz, 2H), 3.13 (dd, J = 16.8 Hz, 2.8 Hz, 1H), 2.92 (dd, J = 16.8 Hz, 4.8 Hz, 1H), 2.47-2.37 (m, 2H), 2.20- 2.10 (m, 3H), 1.90 (d, J = 11.2 Hz, 2H), 1.79-1.72 (m, 3H), 1.23-1.14 (m, 2H), 0.96 (d, J = 6.4 Hz, 3H). |
| I-402 | 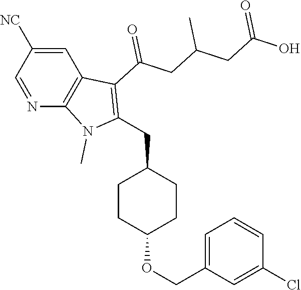 [SMILES: CC(CC(=O)O)CC(=O)c1c(C[C@H]2CC[C@H](OCc3cccc(Cl)c3)CC2)n(C)c2ncc(C#N)cc12] | 522.3 | ¹H NMR (400 MHz, DMSO-d₆ + D₂O) δ 8.81 (d, J = 1.6 Hz, 1H), 8.73 (d, J = 1.6 Hz, 1H), 7.39-7.30 (m, 3H), 7.26 (d, J = 7.2 Hz, 1H), 4.49 (s, 2H), 3.85 (s, 3H), 3.30-3.27 (m, 1H), 3.18 (d, J = 6.4 Hz, 2H), 3.10 (dd, J = 16.8 Hz, 5.6 Hz, 1H), 2.93 (dd, J = 16.0 Hz, 7.2 Hz, 1H), 2.48- 2.46 (m, 1H), 2.43-2.35 (m, 1H), 2.19 (dd, J = 15.6 Hz, 7.6 Hz, 1H), 1.99 (d, J = 6.8 Hz, 2H), 1.71-1.61 (m, 3H), 1.24-1.10 (m, 4H), 0.97 (d, J = 6.4 Hz, 3H). |
| I-403 | 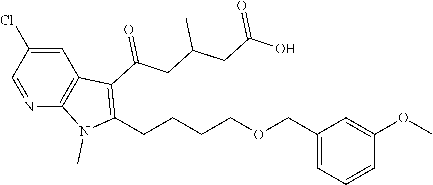 [SMILES: COc1cccc(COCCCCc2c(C(=O)CC(C)CC(=O)O)c3cc(Cl)cnc3n2C)c1] | 487.2 | ¹H NMR (400 MHz, DMSO-d₆) δ 12.12 (br., 1H), 8.37 (d, J = 2.0 Hz, 1H), 8.32 (d, J = 2.0 Hz, 1H), 7.24 (t, J = 4.0 Hz, 1H), 6.89-6.82 (m, 3H), 4.43 (s, 2H), 3.80 (s, 3H), 3.73 (s, 3H), 3.49 (t, J = 5.6 Hz, 2H), 3.22 (t, J = 6.8 Hz, 2H), 3.04 (dd, J = 16.8 Hz, 6.0 Hz, 1H), 2.87 (dd, J = 16.8 Hz, 8.0 Hz, 1H), 2.51-2.47 (m, 1H), 2.40 (dd, J = 15.6 Hz, 6.0 Hz, 1H), 2.18 (dd, J = 15.6 Hz, 8.0 Hz, 1H), 1.67-1.69 (m, 4H), 0.97 (d, J = 6.8 Hz, 3H). |
| I-404 | 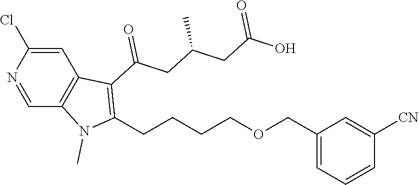 [SMILES: C[C@H](CC(=O)O)CC(=O)c1c(CCCCOCc2cccc(C#N)c2)n(C)c2cnc(Cl)cc12] | 482.2 | ¹H NMR (400 MHz, DMSO-d₆) δ 12.02 (br., 1H), 8.74 (s, 1H), 7.88 (s, 1H), 7.77- 7.73 (m, 2H), 7.67 (d, J = 7.6 Hz, 1H), 7.56 (t, J = 8.0 Hz, 1H), 4.52 (s, 2H), 3.86 (s, 3H), 3.52 (t, J = 6.0 Hz, 2H), 3.21 (t, J = 8.0 Hz, 2H), 3.02 (dd, J = 16.4 Hz, 5.6 Hz, 1H), 2.86 (dd, J = 16.4 Hz, 7.6 Hz, 1H), 2.48-2.43 (m, 1H), 2.38 (dd, J = 15.6 Hz, 5.6 Hz, 1H), 2.17 (dd, J = 15.2 Hz, 7.6 Hz, 1H), 1.73-1.61 (m, 4H), 0.95 (d, J = 6.8 Hz, 3H). |
| I-405 | 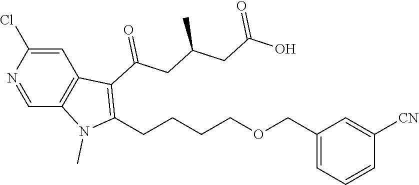 [SMILES: C[C@@H](CC(=O)O)CC(=O)c1c(CCCCOCc2cccc(C#N)c2)n(C)c2cnc(Cl)cc12] | 482.1 | ¹H NMR (400 MHz, DMSO-d₆) δ 12.03 (br., 1H), 8.74 (s, 1H), 7.88 (s, 1H), 7.77- 7.73 (m, 2H), 7.56 (t, J = 8.0 Hz, 1H), 4.52 (s, 2H), 3.86 (s, 3H), 3.52 (t, J = 6.0 Hz, 2H), 3.21 (t, J = 6.0 Hz, 2H), 3.03 (dd, J = 16.8 Hz, 5.6 Hz, 1H), 2.86 (dd, J = 16.8 Hz, 8.0 Hz, 1H), 2.48-2.43 (m, 1H), 2.38 (dd, J = 15.6 Hz, 6.0 Hz, 1H), 2.17 (dd, J = 15.6 Hz, 8.0 Hz, 1H), 1.73-1.61 (m, , 4H), 0.95(d, J = 6.8 Hz, 3H). |
| I-406 | 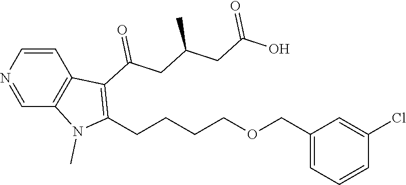 [SMILES: C[C@@H](CC(=O)O)CC(=O)c1c(CCCCOCc2cccc(Cl)c2)n(C)c2cnccc12] | 457.1 | ¹H NMR (400 MHz, DMSO-d₆) δ 12.07 (br., 1H), 8.88 (s, 1H), 8.27 (d, J = 5.2 Hz, 1H), 7.80 (d, J = 5.2 Hz, 1H), 7.36-7.24 (m, 4H), 4.44 (s, 2H), 3.84 (s, 3H), 3.47 (t, J = 5.6 Hz, 2H), 3.20 (t, J = 7.6 Hz, 2H), 3.00 (dd, J = 16.4 Hz, 6.0 Hz, 1H), 2.84 (dd, J = 16.4 Hz, 8.0 Hz, 1H), 2.47- 2.41 (m, 1H), 2.36 (dd, J = 15.6 Hz, 5.6 Hz, 1H), 2.13 (dd, J = 15.6 Hz, 8.0 Hz, 1H), 1.69-1.58 (m, 4H), 0.94 (d, J = 6.4 Hz, 3H). |
| I-407 | 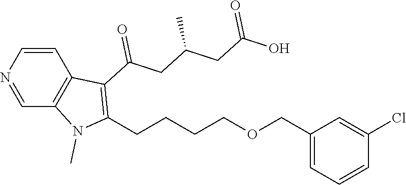 [SMILES: C[C@H](CC(=O)O)CC(=O)c1c(CCCCOCc2cccc(Cl)c2)n(C)c2cnccc12] | 457.1 | ¹H NMR (400 MHz, DMSO-d₆) δ 12.08 (s, 1H), 8.88 (s, 1H), 8.27 (d, J = 5.6 Hz, 1H), 7.80 (d, J = 5.6 Hz, 1H), 7.36-7.24 (m, 4H), 4.44 (s, 2H), 3.84 (s, 3H), 3.47 (t, J = 5.6 Hz, 2H), 3.20 (t, J = 7.6 Hz, 2H), 3.00 (dd, J = 16.4 Hz, 6.0 Hz, 1H), 2.84 (dd, J = 16.0 Hz, 7.6 Hz, 1H), 2.46- 2.41 (m, 1H), 2.36 (dd, J = 15.2 Hz, 5.6 Hz, 1H), 2.13 (dd, J = 15.2 Hz, 8.0 Hz, 1H), 1.70-1.58 (m, 4H), 0.94 (d, J = 6.4 Hz, 3H). |
| I-408 | 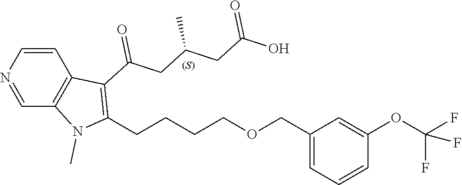 | 507.3 | ¹H NMR (400 MHz, DMSO-d₆) δ 12.13 (br., 1H), 8.91 (s, 1H), 8.30 (d, J = 5.6 Hz, 1H), 7.83 (d, J = 5.6 Hz, 1H), 7.47 (t, J = 8.0 Hz, 1H), 7.35 (d, J = 7.6 Hz, 1H), 7.28-7.25 (m, 2H), 4.52 (s, 2H), 3.86 (s, 3H), 3.52 (t, J = 5.6 Hz, 2H), 3.23 (t, J = 7.6 Hz, 2H), 3.03 (dd, J = 16.0 Hz, 5.6 Hz, 1H), 2.87 (dd, J = 16.4 Hz, 7.6 Hz, 1H), 2.51-2.49 (m, 1H), 2.38 (dd, J = 15.2 Hz, 5.6 Hz, 1H), 2.16 (dd, J = 15.2 Hz, 8.0 Hz, 1H), 1.72-1.64 (m, 4H), 0.96 (d, J = 6.4 Hz, 3H). |
| I-409 | 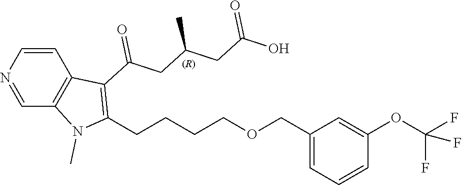 | 507.1 | ¹H NMR (400 MHz, DMSO-d₆) δ 8.90 (s, 1H), 8.29 (d, J = 5.2 Hz, 1H), 7.83 (d, J = 5.2 Hz, 1H), 7.47 (t, J = 8.0 Hz, 1H), 7.35 (d, J = 7.6 Hz, 1H), 7.28-7.25 (m, 2H), 4.52 (s, 2H), 3.86 (s, 3H), 3.51 (t, J = 5.6 Hz, 2H), 3.23 (t, J = 7.6 Hz, 2H), 3.05 (dd, J = 16.0 Hz, 5.2 Hz, 1H), 2.83 (dd, J = 16.0 Hz, 7.6 Hz, 1H), 2.50-2.45 (m, 1H), 2.34-2.29 (m, 1H), 2.11 (dd, J = 15.2 Hz, 8.0 Hz, 1H), 1.71-1.63 (m, 4H), 0.95 (d, J = 6.8 Hz, 3H). |
| I-410 | 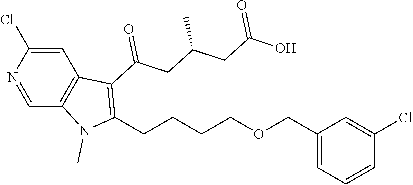 [SMILES: C[C@H](CC(=O)O)CC(=O)c1c(CCCCOCc2cccc(Cl)c2)n(C)c2cnc(Cl)cc12] | 491.2 | ¹H NMR (400 MHz, DMSO-d₆) δ 12.08 (br., 1H), 8.74 (s, 1H), 7.88 (s, 1H), 7.39- 7.32 (m, 3H), 7.29-7.27 (m, 1H), 4.47 (s, 2H), 3.86 (s, 3H), 3.50 (t, J = 6.0 Hz, 2H), 3.21 (t, J = 7.6 Hz, 2H), 3.02 (dd, J = 16.4 Hz, 6.0 Hz, 1H), 2.86 (dd, J = 16.8 Hz, 7.6 Hz, 1H), 2.49-2.46 (m, 1H), 2.41 (dd, J = 15.2 Hz, 5.6 Hz, 1H), 2.18 (dd, J = 15.6 Hz, 8.0 Hz, 1H), 1.72-1.64 (m, 4H), 0.96 (d, J = 6.4 Hz, 3H). |
| I-411 | 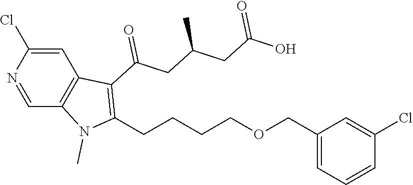 [SMILES: C[C@@H](CC(=O)O)CC(=O)c1c(CCCCOCc2cccc(Cl)c2)n(C)c2cnc(Cl)cc12] | 491.2 | ¹H NMR (400 MHz, DMSO-d₆) δ 12.08 (br., 1H), 8.74 (s, 1H), 7.88 (s, 1H), 7.39- 7.33 (m, 3H), 7.29-7.27 (m, 1H), 4.47 (s, 2H), 3.86 (s, 3H), 3.50 (t, J = 6.0 Hz, 2H), 3.21 (t, J = 8.0 Hz, 2H), 3.02 (dd, J = 16.8 Hz, 5.6 Hz, 1H), 2.86 (dd, J = 16.8 Hz, 7.6 Hz, 1H), 2.49-2.46 (m, 1H), 2.41 (dd, J = 15.2 Hz, 5.6 Hz, 1H), 2.18 (dd, J = 15.6 Hz, 8.0 Hz, 1H), 1.72-1.64 (m, 4H), 0.96 (d, J = 6.4 Hz, 3H). |
| I-412 | 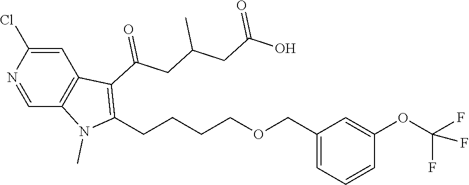 [SMILES: CC(CC(=O)O)CC(=O)c1c(CCCCOCc2cccc(OC(F)(F)F)c2)n(C)c2cnc(Cl)cc12] | 541.2 | ¹H NMR (400 MHz, DMSO-d₆) δ 8.74 (s, 1H), 7.88 (s, 1H), 7.47 (t, J = 7.6 Hz, 1H), 7.36-7.34 (m, 1H), 7.28-7.25 (m, 2H), 4.52 (s, 2H), 3.85 (s, 3H), 3.51 (t, J = 6.0 Hz, 2H), 3.23-3.20 (m, 2H), 3.03 (dd, J = 16.4 Hz, 5.6 Hz, 1H), 2.85 (dd, J = 16.8 Hz, 7.6 Hz, 1H), 2.48-2.46 (m, 1H), 2.38 (dd, J = 15.2 Hz, 5.6 Hz, 1H), 2.17 (dd, J = 15.6 Hz, 8.0 Hz, 1H), 1.72-1.64 (m, 4H) , 0.95 (d, J = 6.4 Hz, 3H). |
| I-413 | 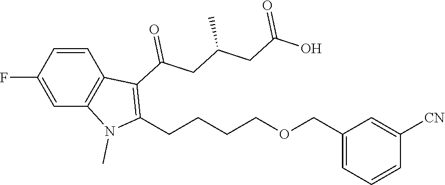 [SMILES: C[C@H](CC(=O)O)CC(=O)c1c(CCCCOCc2cccc(C#N)c2)n(C)c2cc(F)ccc12] | 465.3 | ¹H NMR (400 MHz, DMSO-d₆) δ 12.06 (br., 1H), 7.89 (dd, J = 8.8 Hz, 5.6 Hz, 1H), 7.73-7.71 (m, 2H), 7.64 (d, J = 7.6 Hz, 1H), 7.55-7.50 (m, 1H), 7.43 (dd, J = 10.0 Hz, 2.4 Hz, 1H), 7.03 (dt, J = 9.2 Hz, 2.4 Hz, 1H), 4.49 (s, 2H), 3.69 (s, 3H), 3.49 (t, J = 6.0 Hz, 2H), 3.14 (t, J = 7.2 Hz, 2H), 2.96 (dd, J = 16.4 Hz, 6.0 Hz, 1H), 2.82 (dd, J = 16.4 Hz, 7.6 Hz, 1H), 2.49-2.43 (m, 1H), 2.35 (dd, J = 15.2 Hz, 5.6 Hz, 1H), 2.12 (dd, J = 15.2 Hz, 8.0 Hz, 1H), 1.69-1.59 (m, 4H), 0.93 (d, J = 6.8 Hz, 3H). |
| I-414 | 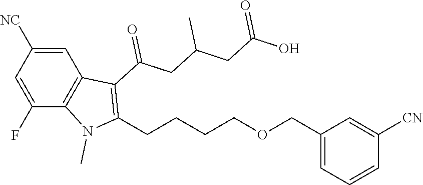 [SMILES: CC(CC(=O)O)CC(=O)c1c(CCCCOCc2cccc(C#N)c2)n(C)c2c(F)cc(C#N)cc12] | 490.3 | ¹H NMR (400 MHz, DMSO-d₆) δ 12.12 (br., 1H), 8.30 (s, 1H), 7.75 (d, J = 7.2 Hz, 2H), 7.67 (d, J = 8.0 Hz, 1H), 7.60-7.54 (m, 2H), 4.52 (s, 2H), 3.94 (s, 3H), 3.53 (t, J = 6.0 Hz, 2H), 3.18 (t, J = 7.2 Hz, 2H), 3.07 (dd, J = 16.4 Hz, 5.2 Hz, 1H), 2.90 (dd, J = 16.8 Hz, 7.6 Hz, 1H), 2.48- 2.46 (m, 1H), 2.39 (dd, J = 15.6 Hz, 5.6 Hz, 1H), 2.17 (dd, J = 15.6 Hz, 8.0 Hz, 1H), 1.74-1.65 (m, 4H), 0.95 (d, J = 6.8 Hz, 3H). |
| I-415 | 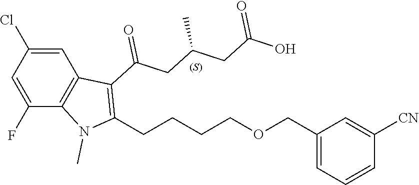 | 499.2 | ¹H NMR (400 MHz, DMSO-d₆) δ 12.09 (s, 1H), 7.83 (d, J = 1.2 Hz, 1H), 7.76-7.74 (m, 2H), 7.67 (d, J = 8.0 Hz, 1H), 7.56 (t, J = 8.0 Hz, 1H), 7.23 (dd, J = 12.0 Hz, 1.2 Hz, 1H), 4.52 (s, 2H), 3.90 (s, 3H), 3.52 (t, J = 5.6 Hz, 2H), 3.15 (t, J = 8.0 Hz, 2H), 2.99 (dd, J = 16.4 Hz, 5.6 Hz, 1H), 2.84 (dd, J = 16.4 Hz, 7.6 Hz, 1H), 2.50- 2.46 (m, 1H), 2.37 (dd, J = 15.2 Hz, 5.6 Hz, 1H), 2.16 (dd, J = 15.6 Hz, 7.6 Hz, 1H), 1.73-1.64 (m, 4H), 0.95 (d, J = 6.4 Hz, 3H). |
| I-416 | 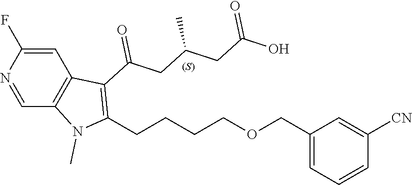 | 466.3 | ¹H NMR (400 MHz, DMSO-d₆) δ 12.10 (s, 1H), 8.32 (dd, J = 2.4 Hz, 1.2 Hz, 1H), 8.18 (dd, J = 10.0 Hz, 2.4 Hz, 1H), 7.77- 7.73 (m, 2H), 7.67 (d, J = 7.6 Hz, 1H), 7.56 (t, J = 7.6 Hz, 1H), 4.52 (s, 2H), 3.81 (s, 3H), 3.52 (t, J = 5.6 Hz, 2H), 3.23 (t, J = 7.6 Hz, 2H), 3.03 (dd, J = 16.4 Hz, 5.6 Hz, 1H), 2.84 (dd, J = 16.4 Hz, 7.6 Hz, 1H), 2.45-2.44 (m, 1H), 2.41 (dd, J = 15.2 Hz, 5.6 Hz, 1H), 2.16 (dd, J = 15.6 Hz, 8.0 Hz, 1H), 1.74-1.65 (m, 4H), 0.95 (d, J = 6.4 Hz, 3H). |
| I-417 | 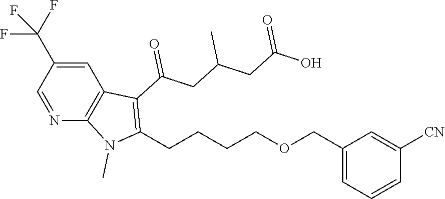 [SMILES: CC(CC(=O)O)CC(=O)c1c(CCCCOCc2cccc(C#N)c2)n(C)c2ncc(C(F)(F)F)cc12] | 516.3 | ¹H NMR (400 MHz, DMSO-d₆) δ 12.12 (s, 1H), 8.69 (s, 1H), 8.63 (s, 1H), 7.76- 7.74 (m, 2H), 7.67 (d, J = 8.0 Hz, 1H), 7.56 (t, J = 8.0 Hz, 1H), 4.53 (s, 2H), 3.87 (s, 3H), 3.54 (t, J = 5.2 Hz, 2H), 3.27 (t, J = 7.2 Hz, 2H), 3.11 (dd, J = 16.4 Hz, 5.6 Hz, 1H), 2.92 (dd, J = 16.8 Hz, 7.6 Hz, 1H), 2.49-2.46 (m, 1H), 2.39 (dd, J = 15.6 Hz, 6.0 Hz, 1H), 2.19 (dd, J = 15.6 Hz, 7.6 Hz, 1H), 1.75-1.66 (m, 4H), 0.96 (d, J = 6.4 Hz, 3H). |
| I-418 | 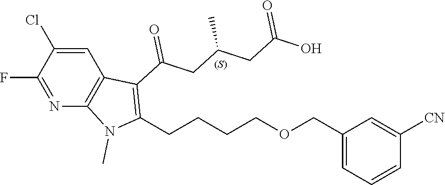 | 500.2 | ¹H NMR (400 MHz, DMSO-d₆) δ 12.11 (s, 1H), 8.60 (d, J = 9.2 Hz, 1H), 7.76-7.74 (m, 2H), 7.67 (d, J = 8.0 Hz, 1H), 7.56 (t, J = 8.0 Hz, 1H), 4.52 (s, 2H), 3.74 (s, 3H), 3.52 (t, J = 5.6 Hz, 2H), 3.20 (t, J = 8.0 Hz, 2H), 3.06 (dd, J = 16.8 Hz, 5.6 Hz, 1H), 2.87 (dd, J = 16.4 Hz, 7.6 Hz, 1H), 2.49-2.45 (m, 1H), 2.39 (dd, J = 15.6 Hz, 5.6 Hz, 1H), 2.17 (dd, J = 15.6 Hz, 8.0 Hz, 1H), 1.75-1.63 (m, 4H), 0.95 (d, J = 6.4 Hz, 3H). |
| I-419 | 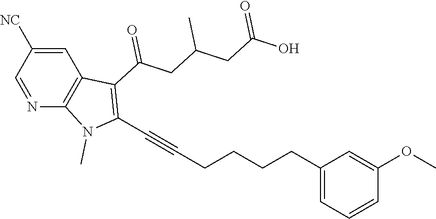 [SMILES: COc1cccc(CCCCC#Cc2c(C(=O)CC(C)CC(=O)O)c3cc(C#N)cnc3n2C)c1] | 472.2 | ¹H NMR (400 MHz, DMSO-d₆) δ 12.09 (br., 1H), 8.87 (s, 1H), 8.83 (s, 1H), 7.19 (t, J = 8.0 Hz, 1H), 6.80-6.79 (m, 2H), 6.74 (d, J = 8.4 Hz, 1H), 3.86 (s, 3H), 3.71 (s, 3H), 3.06 (d, J = 6.8 Hz, 2H), 2.77 (t, J = 6.8 Hz, 2H), 2.63 (t, J = 7.2 Hz, 2H), 2.47 (s, 1H), 2.33 (dd, J = 15.2 Hz, 5.6 Hz, 1H), 2.14 (dd, J = 15.6 Hz, 8.4Hz, 1H), 1.82-1.68 (m, 4H), 0.96 (d, J = 6.4 Hz, 3H). |
| I-420 | 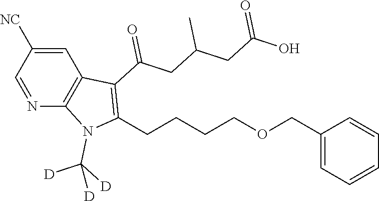 [SMILES: [2H]C([2H])([2H])n1c(CCCCOCc2ccccc2)c(C(=O)CC(C)CC(=O)O)c2cc(C#N)cnc21] | 451.2 | ¹H NMR (400 MHz, DMSO-d₆) δ 8.82 (s, 1H), 8.71 (d, J = 1.2 Hz, 1H), 7.35-7.30 (m, 4H), 7.28-7.25 (m, 1H), 4.46 (s, 2H), 3.50 (t, J = 5.6 Hz, 2H), 3.24 (t, J = 6.8 Hz, 2H), 3.10 (dd, J = 16.8 Hz, 5.2 Hz, 1H), 2.88 (dd, J = 16.8 Hz, 8.0 Hz, 1H), 2.49-2.44 (m, 1H), 2.32 (dd, J = 15.2 Hz, 5.6 Hz, 1H), 2.12 (dd, J = 15.6 Hz, 8.0 Hz, 1H), 1.73-16.4 (m, 4H), 0.96 (d, J = 6.4 Hz, 3H). |
| I-421 | 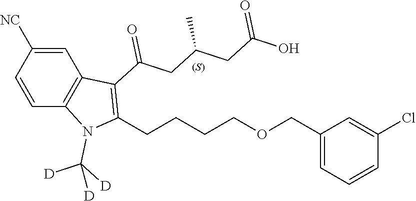 | 484.2 | ¹H NMR (400 MHz, DMSO-d₆) δ 12.08 (s, 1H), 8.43 (d, J = 0.8 Hz, 1H), 7.77 (d, J = 8.8 Hz, 1H), 7.62 (dd, J = 8.4 Hz, 1.6 Hz, 1H), 7.39-7.26 (m, 4H), 4.47 (s, 2H), 3.50 (t, J = 6.0 Hz, 2H), 3.20 (t, J = 8.0 Hz, 2H), 3.07 (dd, J = 16.8 Hz, 5.6 Hz, 1H), 2.91 (dd, J = 16.8 Hz, 7.6 Hz, 1H), 2.56-2.52 (m, 1H), 2.40 (dd, J = 15.6 Hz, 6.0 Hz, 1H), 2.17 (dd, J = 15.6 Hz, 8.0 Hz, 1H), 1.75-1.60 (m, 4H), 0.96 (d, J = 6.8 Hz, 3H). |
| I-422 | 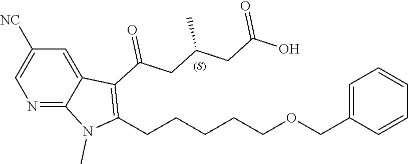 | 462.3 | ¹H NMR (400 MHz, DMSO-d₆) δ 12.20 (br, 1H), 8.84 (d, J = 1.6 Hz, 1H), 8.73 (d, J = 1.6 Hz, 1H), 7.35-7.25 (m, 5H), 4.44 (s, 2H), 3.85 (s, 3H), 3.44 (t, J = 6.0 Hz, 2H), 3.24 (t, J = 8.8 Hz, 2H), 3.10 (dd, J = 16.8 Hz, 5.6 Hz, 1H), 2.89 (dd, J = 16.8 Hz, 8.0 Hz, 1H), 2.49-2.45 (m, 1H), 2.38 (dd, J = 15.6 Hz, 5.6 Hz, 1H), 2.16 (dd, J = 15.6 Hz, 8.0 Hz, 1H), 1.63-1.58 (m, 4H), 1.52-1.48 (m, 2H), 0.96 (d, J = 6.4 Hz, 3H). |
| I-423 | 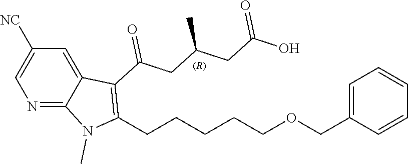 | 462.3 | ¹H NMR (400 MHz, DMSO-d₆) δ 12.20 (br., 1H), 8.83 (s, 3H), 8.73 (d, J = 1.6 Hz, 1H), 7.35-7.25 (m, 5H), 4.44 (s, 2H), 3.85 (s, 3H), 3.44 (t, J = 6.0 Hz, 2H), 3.24 (t, J = 8.8 Hz, 2H), 3.10 (dd, J = 16.8 Hz, 5.6 Hz, 1H), 2.90 (dd, J = 16.8 Hz, 8.0 Hz, 1H), 2.49-2.45 (m, 1H), 2.38 (dd, J = 15.6 Hz, 5.6 Hz, 1H), 2.16 (dd, J = 15.6 Hz, 8.0 Hz, 1H), 1.63-1.58 (m, 4H), 1.52-1.48 (m, 2H), 0.96 (d, J = 6.4 Hz, 3H). |
| I-424 | 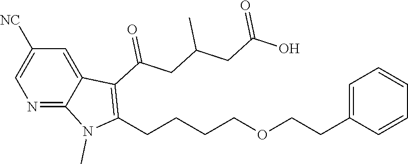 [SMILES: CC(CC(=O)O)CC(=O)c1c(CCCCOCCc2ccccc2)n(C)c2ncc(C#N)cc12] | 462.3 | ¹H NMR (400 MHz, DMSO-d₆) δ 12.09 (br., 1H), 8.83 (d, J = 1.6 Hz, 1H), 8.74 (d, J = 1.6 Hz, 1H), 7.26-7.15 (m, 5H), 3.81 (s, 3H), 3.57 (t, J = 6.8 Hz, 2H), 3.45 (t, J = 5.6 Hz, 2H), 3.21 (t, J = 6.8 Hz, 2H), 3.09 (dd, J = 16.8 Hz, 5.2 Hz, 1H), 2.91 (dd, J = 16.8 Hz, 7.6 Hz, 1H), 2.79 (t, J = 7.2 Hz, 2H), 2.50-2.48 (m, 1H), 2.41 (dd, J = 15.6 Hz, 6.0 Hz, 1H), 2.18 (dd, J = 15.6 Hz, 8.0 Hz, 1H), 1.64-1.61 (m, 4H), 0.97 (d, J = 6.4 Hz, 3H). |
| I-425 | 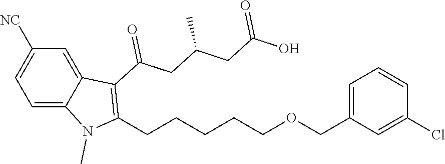 [SMILES: C[C@H](CC(=O)O)CC(=O)c1c(CCCCCOCc2cccc(Cl)c2)n(C)c2ccc(C#N)cc12] | 495.3 | ¹H NMR (400 MHz, DMSO-d₆) δ 12.11 (s, 1H), 8.43 (d, J = 0.4 Hz, 1H), 7.77 (d, J = 8.8 Hz, 1H), 7.62 (dd, J = 8.4 Hz, 1.2 Hz, 1H), 7.39-7.31 (m, 3H), 7.28-7.25 (m, 1H), 4.46 (s, 2H), 3.81 (s, 3H), 3.45 (t, J = 6.0 Hz, 2H), 3.20 (t, J = 8.0 Hz, 2H), 3.07 (dd, J = 16.8 Hz, 5.6 Hz, 1H), 2.91 (dd, J = 16.8 Hz, 7.6 Hz, 1H), 2.56-2.52 (m, 1H), 2.40 (dd, J = 15.6 Hz, 6.0 Hz, 1H), 2.17 (dd, J = 15.6 Hz, 8.0 Hz, 1H), 1.75-1.60 (m, 4H), 1.52-1.45 (m, 2H), 0.96 (d, J = 6.8 Hz, 3H). |
| I-426 | 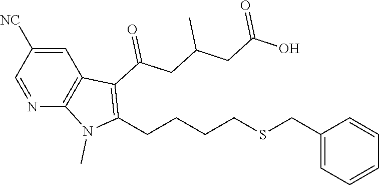 [SMILES: CC(CC(=O)O)CC(=O)c1c(CCCCSCc2ccccc2)n(C)c2ncc(C#N)cc12] | 464.3 | ¹H NMR (400 MHz, CD₃OD) δ 8.77 (d, J = 1.6 Hz, 1H), 8.60 (d, J = 1.6 Hz, 1H), 7.29-7.22 (m, 4H), 7.14 (t, J = 7.2 Hz, 1H), 3.87 (s, 3H), 3.70 (s, 2H), 3.19 (t, J = 6.4 Hz, 2H), 3.14 (dd, J = 8.4 Hz, 4.8 Hz, 1H), 2.82 (dd, J = 16.0 Hz, 8.8 Hz, 1H), 2.65-2.60 (m, 1H), 2.50 (t, J = 6.4 Hz, 2H), 2.26 (dd, J = 13.6 Hz, 7.2 Hz, 1H), 2.19 (dd, J = 13.2 Hz, 6.8 Hz, 1H), 1.72-1.67 (m, 4H), 1.04 (d, J = 6.4 Hz, 3H). |
| I-427 | 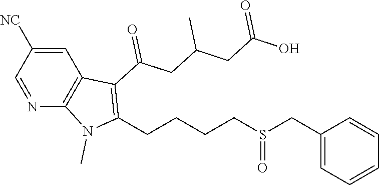 [SMILES: CC(CC(=O)O)CC(=O)c1c(CCCCS(=O)Cc2ccccc2)n(C)c2ncc(C#N)cc12] | 480.3 | ¹H NMR (400 MHz, DMSO-d₆) δ 8.84 (d, J = 1.6 Hz, 1H), 8.74 (d, J = 2.0 Hz, 1H), 7.38-7.30 (m, 5H), 4.13 (d, J = 12.8 Hz, 1H), 3.96 (d, J = 12.8 Hz, 1H), 3.86 (s, 3H), 3.28 (t, J = 6.8 Hz, 2H), 3.11 (dd, J = 16.8 Hz, 5.2 Hz, 1H), 2.92 (dd, J = 16.8 Hz, 7.6 Hz, 1H), 2.85-2.78 (m, 1H), 2.71-2.65 (m, 1H), 2.55-2.51 (m, 1H), 2.41 (dd, J = 15.2 Hz, 5.6 Hz, 1H), 2.19 (dd, J = 15.2 Hz, 7.6 Hz, 1H), 1.83-1.69 (m, 4H), 0.97 (d, J = 6.8 Hz, 3H). |
| I-428 | 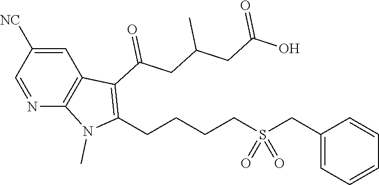 [SMILES: CC(CC(=O)O)CC(=O)c1c(CCCCS(=O)(=O)Cc2ccccc2)n(C)c2ncc(C#N)cc12] | 496.2 | ¹H NMR (400 MHz, DMSO-d₆) δ 12.02 (br., 1H), 8.85 (d, J = 1.6 Hz, 1H), 8.75 (d, J = 1.6 Hz, 1H), 7.41-7.33 (m, 5H), 4.46 (d, J = 7.6 Hz, 2H), 3.86 (s, 3H), 3.24 (t, J = 8.4 Hz, 2H), 3.13-3.08 (m, 3H), 2.93 (dd, J = 16.8 Hz, 7.6 Hz, 1H), 2.55-2.51 (m, 1H), 2.43 (dd, J = 15.6 Hz, 6.0 Hz, 1H), 2.19 (dd, J = 15.2 Hz, 8.0 Hz, 1H), 1.87-1.79 (m, 2H), 1.74-1.68 (m, 2H), 0.97 (d, J = 6.8 Hz, 3H). |
| I-429 | 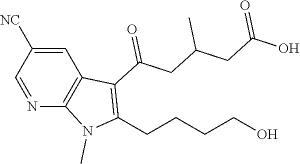 [SMILES: CC(CC(=O)O)CC(=O)c1c(CCCCO)n(C)c2ncc(C#N)cc12] | 358.3 | ¹H NMR (400 MHz, DMSO-d₆) δ 12.08 (s, 1H), 8.83 (d, J = 2.0 Hz, 1H), 8.73 (d, J = 1.6 Hz, 1H), 4.43 (t, J = 4.8 Hz, 1H), 3.87 (s, 3H), 3.48-3.43 (m, 2H), 3.23 (t, J = 7.2 Hz, 2H), 3.09 (dd, J = 16.8 Hz, 5.2 Hz, 1H), 2.92 (dd, J = 16.8 Hz, 7.6 Hz, 1H), 2.49-2.46 (m, 1H), 2.42 (dd, J = 15.6 Hz, 6.0 Hz, 1H), 2.19 (dd, J = 15.2 Hz, 8.0 Hz, 1H), 1.67-1.55 (m, 4H), 0.98 (d, J = 6.4 Hz, 3H). |
| I-430 | 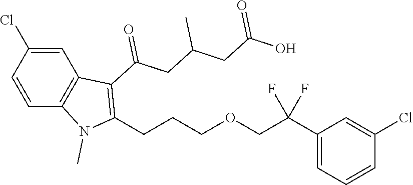 [SMILES: CC(CC(=O)O)CC(=O)c1c(CCCOCC(F)(F)c2cccc(Cl)c2)n(C)c2ccc(Cl)cc12] | 526.2 | ¹H NMR (400 MHz, DMSO-d₆) δ 12.04 (s, 1H), 7.95 (d, J = 1.6 Hz, 1H), 7.63-7.53 (m, 5H), 7.25 (dd, J = 8.8 Hz, 2.0 Hz, 1H), 4.03 (t, J = 13.6 Hz, 2H), 3.70 (s, 3H), 3.58 (t, J = 6.4 Hz, 2H), 3.10 (t, J = 7.6 Hz, 2H), 2.97 (dd, J = 16.4 Hz, 5.6 Hz, 1H), 2.84 (dd, J = 16.4 Hz, 7.6 Hz, 1H), 2.48-2.43 (m, 1H), 2.38 (dd, J = 15.6 Hz, 5.6 Hz, 1H), 2.16 (dd, J = 15.6 Hz, 8.0 Hz, 1H), 1.83-1.75 (m, 2H), 0.96 (d, J = 6.8 Hz, 3H). |
| I-431 | 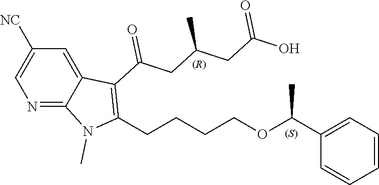 | 462.3 | ¹H NMR (400 MHz, DMSO-d₆) δ 12.07 (br., 1H), 8.83 (d, J = 1.6 Hz, 1H), 8.73 (d, J = 1.6 Hz, 1H), 7.35-7.22 (m, 5H), 4.41 (q, J = 6.4 Hz, 1H), 3.84 (s, 3H), 3.31-3.16 (m, 4H), 3.09 (dd, J = 16.8 Hz, 5.6 Hz, 1H), 2.90 (dd, J = 16.8 Hz, 8.0 Hz, 1H), 2.47-2.36 (m, 2H), 2.17 (dd, J = 15.6 Hz, 8.0 Hz, 1H), 1.68-1.58 (m, 4H), 1.32 (d, J = 6.8 Hz, 3H), 0.96 (d, J = 6.8 Hz, 3H). |
| I-432 | 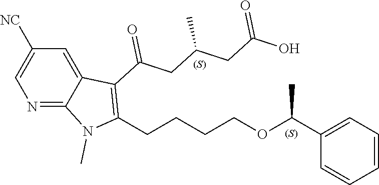 | 462.3 | ¹H NMR (400 MHz, DMSO-d₆) δ 12.09 (br., 1H), 8.83 (d, J = 1.6 Hz, 1H), 8.73 (d, J = 2.0 Hz, 1H), 7.35-7.24 (m, 5H), 4.41 (q, J = 6.4 Hz, 1H), 3.84 (s, 3H), 3.31-3.16 (m, 4H), 3.09 (dd, J = 16.8 Hz, 5.6 Hz, 1H), 2.91 (dd, J = 16.8 Hz, 7.6 Hz, 1H), 2.47-2.36 (m, 2H), 2.18 (dd, J = 15.6 Hz, 8.0 Hz, 1H), 1.68-1.58 (m, 4H), 1.32 (d, J = 6.4 Hz, 3H), 0.96 (d, J = 6.8 Hz, 3H). |
| I-433 | 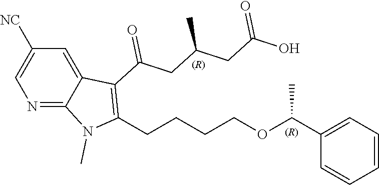 | 462.3 | ¹H NMR (400 MHz, DMSO-d₆) δ 12.09 (s, 1H), 8.83 (d, J = 1.6 Hz, 1H), 8.73 (d, J = 2.0 Hz, 1H), 7.35-7.22 (m, 5H), 4.41 (q, J = 6.4 Hz, 1H), 3.84 (s, 3H), 3.31-3.16 (m, 4H), 3.09 (dd, J = 16.8 Hz, 5.6 Hz, 1H), 2.91 (dd, J = 16.8 Hz, 7.6 Hz, 1H), 2.47-2.36 (m, 2H), 2.18 (dd, J = 15.6 Hz, 8.0 Hz, 1H), 1.68-1.58 (m, 4H), 1.32 (d, J = 6.4 Hz, 3H), 0.97 (d, J = 6.4 Hz, 3H). |
| I-434 | 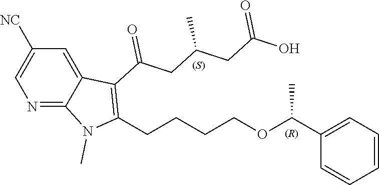 | 462.3 | ¹H NMR (400 MHz, DMSO-d₆) δ 12.06 (br., 1H), 8.83 (d, J = 2.0 Hz, 1H), 8.73 (d, J = 1.6 Hz, 1H), 7.35-7.24 (m, 5H), 4.41 (q, J = 6.4 Hz, 1H), 3.84 (s, 3H), 3.31-3.16 (m, 4H), 3.09 (dd, J = 16.8 Hz, 5.6 Hz, 1H), 2.91 (dd, J = 16.8 Hz, 8.0 Hz, 1H), 2.47-2.36 (m, 2H), 2.18 (dd, J = 15.6 Hz, 8.0 Hz, 1H), 1.68-1.58 (m, 4H), 1.32 (d, J = 6.4 Hz, 3H), 0.96 (d, J = 6.4 Hz, 3H). |
| I-435 | 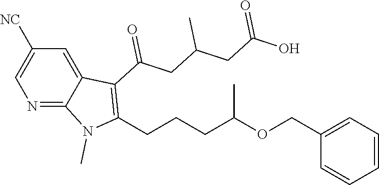 [SMILES: CC(CC(=O)O)CC(=O)c1c(CCCC(C)OCc2ccccc2)n(C)c2ncc(C#N)cc12] | 462.3 | ¹H NMR (400 MHz, DMSO-d₆) δ 12.09 (s, 1H), 8.83 (d, J = 2.0 Hz, 1H), 8.73 (d, J = 2.0 Hz, 1H), 7.31-7.29 (m, 4H), 7.26- 7.23 (m, 1H), 4.52 (d, J = 12.0 Hz, 1H), 4.40 (d, J = 11.6 Hz, 1H), 3.83 (s, 3H), 3.61-3.53 (m, 1H), 3.24 (t, J = 6.8 Hz, 2H), 3.10 (dd, J = 16.8 Hz, 5.6 Hz, 1H), 2.92 (dd, J = 16.8 Hz, 7.6 Hz, 1H), 2.49- 2.36 (m, 2H), 2.18 (dd, J = 15.6 Hz, 8.0 Hz, 1H), 1.73-1.55 (m, 4H), 1.15 (d, J = 6.4 Hz, 3H), 0.96 (d, J = 6.0 Hz, 3H). |
| I-436 | 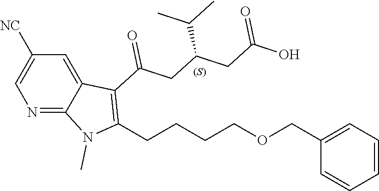 | 476.2 | ¹H NMR (400 MHz, DMSO-d₆) δ 11.92 (br., 1H), 8.83 (d, J = 2.0 Hz, 1H), 8.73 (d, J = 2.0 Hz, 1H), 7.33-7.26 (m, 5H), 4.46 (s, 2H), 3.84 (s, 3H), 3.49 (t, J = 5.2 Hz, 2H), 3.23 (t, J = 6.8 Hz, 2H), 3.00- 2.96 (m, 6.8 Hz, 2H), 2.50-2.48 (m, 1H), 2.26-2.22 (m, 2H), 1.77-1.69 (m, 5H), 0.85 (t, J = 7.2 Hz, 6H). |
| I-437 | 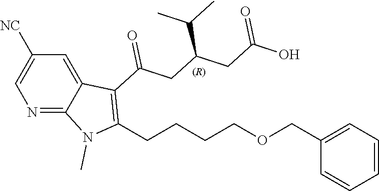 | 476.2 | ¹H NMR (400 MHz, DMSO-d₆) δ 11.87 (br., 1H), 8.83 (d, J = 2.0 Hz, 1H), 8.73 (d, J = 2.0 Hz, 1H), 7.35-7.26 (m, 5H), 4.46 (s, 2H), 3.84 (s, 3H), 3.49 (t, J = 5.2 Hz, 2H), 3.23 (t, J = 7.2 Hz, 2H), 3.00- 2.96 (m, 2H), 2.50-2.45 (m, 1H), 2.26- 2.22 (m, 2H), 1.78-1.69 (m, 5H), 0.85 (t, J = 7.6 Hz, 6H). |
| I-438 | 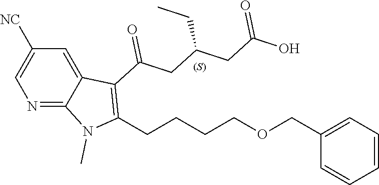 | 462.3 | ¹H NMR (400 MHz, DMSO-d₆) δ 12.09 (br., 1H), 8.82 (d, J = 1.6 Hz, 1H), 8.72 (d, J = 2.0 Hz, 1H), 7.36-7.24 (m, 5H), 4.46 (s, 2H), 3.84 (s, 3H), 3.50 (t, J = 5.6 Hz, 2H), 3.24 (t, J = 7.2 Hz, 2H), 3.08- 2.96 (m, 2H), 2.44-2.24 (m, 3H), 1.74- 1.63 (m, 4H), 1.46-1.32 (m, 2H), 0.85 (t, J = 7.6 Hz, 3H). |
| I-439 | 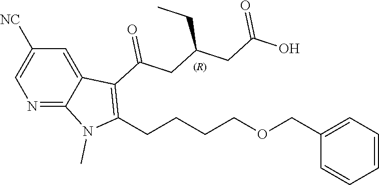 | 462.3 | ¹H NMR (400 MHz, DMSO-d₆) δ 12.03 (br., 1H), 8.83 (d, J = 2.0 Hz, 1H), 8.73 (d, J = 1.6 Hz, 1H), 7.34-7.26 (m, 5H), 4.46 (s, 2H), 3.84 (s, 3H), 3.49 (t, J = 5.6 Hz, 2H), 3.24 (t, J = 7.2 Hz, 2H), 3.03- 3.01 (m, 2H), 2.41-2.27 (m, 3H), 1.73- 1.64 (m, 4H), 1.44-1.32 (m, 2H), 0.85 (t, J = 7.2 Hz, 3H). |
| I-440 | 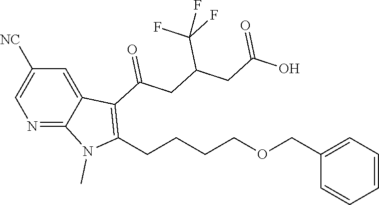 [SMILES: Cn1c(CCCCOCc2ccccc2)c(C(=O)CC(CC(=O)O)C(F)(F)F)c2cc(C#N)cnc21] | 502.1 | ¹H NMR (400 MHz, DMSO-d₆) δ 8.55 (d, J = 2.0 Hz, 1H), 8.31 (d, J = 2.0 Hz, 1H), 7.27-7.23 (m, 4H), 7.18-7.12 (m, 1H), 4.09 (s, 2H), 4.04 (t, J = 6.4 Hz, 2H), 3.79 (s, 3H), 3.20-3.14 (m, 1H), 2.90 (t, J = 7.6 Hz, 2H), 2.59-2.55 (m, 2H), 2.48-2.39 (m, 2H), 1.67-1.60 (m, 2H), 1.54-1.46 (m, 2H). |
| I-441 | 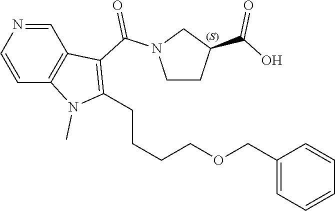 | 436.1 | ¹H NMR (400 MHz, DMSO-d₆) δ 12.50 (br., 1H), 8.65 (s, 1H), 8.22 (d, J = 5.6 Hz, 1H), 7.51 (d, J = 5.4 Hz, 1H), 7.35-7.24 (m, 5H), 4.43 (s, 2H), 3.73 (s, 3H), 3.67- 3.41 (m, 6H), 3.05 (br., 1H), 2.90 (t, J = 7.2 Hz, 2H), 2.18-1.91 (m, 2H), 1.68-1.52 (m, 4H). |
| I-442 | 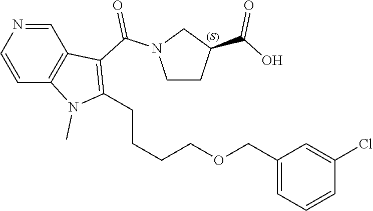 | 470.2 | ¹H NMR (400 MHz, DMSO-d₆) δ 8.64 (s, 1H), 8.22 (d, J = 5.6 Hz, 1H), 7.51 (d, J = 5.6 Hz, 1H), 7.38-7.25 (m, 4H), 4.44 (s, 2H), 3.74 (s, 3H), 3.69-3.41 (m, 6H), 3.05 (br., 1H), 2.90 (t, J = 7.2 Hz, 2H), 2.05 (br., 2H), 1.67-1.55 (m, 4H). |
| I-443 | 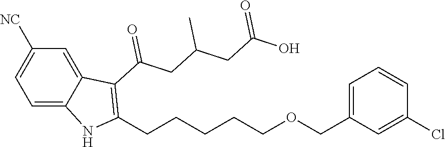 [SMILES: CC(CC(=O)O)CC(=O)c1c(CCCCCOCc2cccc(Cl)c2)[nH]c2ccc(C#N)cc12] | 481.1 | ¹H NMR (400 MHz, DMSO-d₆) δ 12.34 (s, 1H), 12.13 (br., 1H), 8.41 (s, 1H), 7.57- 7.52 (m, 2H), 7.37-7.32 (m, 3H), 7.24 (d, J = 6.8 Hz, 1H), 4.44 (s, 2H), 3.43 (t, J = 6.4 Hz, 2H), 3.10 (t, J = 7.6 Hz, 2H). 3.02 (dd, J = 16.4 Hz, 5.6 Hz, 1H), 2.88 (dd, J = 16.4 Hz, 7.6 Hz, 1H), 2.48-2.45 (m, 1H), 2.40 (dd, J = 15.6 Hz, 6.0 Hz, 1H), 2.17 (dd, J = 15.2 Hz, 8.0 Hz, 1H), 1.74- 1.67 (m, 2H), 1.63-1.53 (m, 2H), 1.44- 1.34 (m, 2H), 0.97 (d, J = 6.4 Hz, 3H). |
| I-444 | 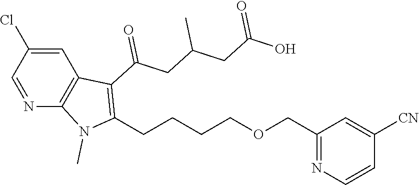 [SMILES: CC(CC(=O)O)CC(=O)c1c(CCCCOCc2cc(C#N)ccn2)n(C)c2ncc(Cl)cc12] | 483.1 | ¹H NMR (400 MHz, DMSO-d₆) δ 12.08 (s, 1H), 8.78 (dd, J = 4.8 Hz, 0.8 Hz, 1H), 8.37 (d, J = 2.0 Hz, 1H), 8.32 (d, J = 2.4 Hz, 1H), 7.80 (s, 1H), 7.77 (dd, J = 5.2 Hz, 1.6 Hz, 1H), 4.64 (s, 2H), 3.82 (s, 3H), 3.61 (t, J = 6.0 Hz, 2H), 3.24 (t, J = 7.6 Hz, 2H), 3.05 (dd, J = 16.8 Hz, 5.6 Hz, 1H), 2.87 (dd, J = 16.8 Hz, 8.0 Hz, 1H), 2.51-2.45 (m, 1H), 2.40 (dd, J = 15.2 Hz, 5.6 Hz, 1H), 2.18 (dd, J = 15.2 Hz, 8.0 Hz, 1H), 1.80-1.68 (m, 4H), 0.97 (d, J = 6.4 Hz, 3H). |
| I-445 | 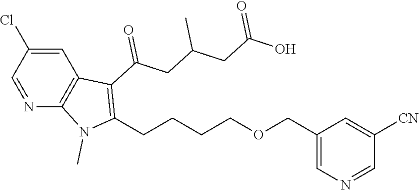 [SMILES: CC(CC(=O)O)CC(=O)c1c(CCCCOCc2cncc(C#N)c2)n(C)c2ncc(Cl)cc12] | 483.1 | ¹H NMR (400 MHz, DMSO-d₆) δ 12.08 (s, 1H), 8.95 (d, J = 2.0 Hz, 1H), 8.82 (d, J = 2.0 Hz, 1H), 8.38 (d, J = 2.4 Hz, 1H), 8.33 (d, J = 2.4 Hz, 1H), 8.25 (t, J = 2.0 Hz, 1H), 4.57 (s, 2H), 3.81 (s, 3H), 3.55 (t, J = 6.0 Hz, 2H), 3.22 (t, J = 7.6 Hz, 2H), 3.04 (dd, J = 16.8 Hz, 5.6 Hz, 1H), 2.86 (dd, J = 16.4 Hz, 7.6 Hz, 1H), 2.49- 2.44 (m, 1H), 2.38 (dd, J = 15.6 Hz, 6.0 Hz, 1H), 2.17 (dd, J = 15.6 Hz, 8.0 Hz, 1H), 1.74-1.63 (m, 4H), 0.95 (d, J = 6.4 Hz, 3H). |
| I-446 | 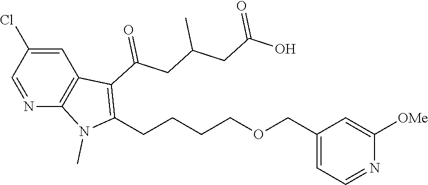 [SMILES: COc1cc(COCCCCc2c(C(=O)CC(C)CC(=O)O)c3cc(Cl)cnc3n2C)ccn1] | 488.2 | ¹H NMR (400 MHz, DMSO-d₆) δ 12.00 (br., 1H), 8.38 (d, J = 2.0 Hz, 1H), 8.33 (d, J = 2.4 Hz, 1H), 8.10 (dd, J = 5.6 Hz, 0.8 Hz, 1H), 6.90 (dd, J = 5.2 Hz, 1.2 Hz, 1H), 6.71 (s, 1H), 4.47 (s, 2H), 3.82 (s, 3H), 3.81 (s, 3H), 3.51 (t, J = 5.6 Hz, 2H), 3.23 (t, J = 7.2 Hz, 2H), 3.04 (dd, J = 16.4 Hz, 5.6 Hz, 1H), 2.87 (dd, J = 16.4 Hz, 7.6 Hz, 1H), 2.48-2.44 (m, 1H), 2.40 (dd, J = 15.2 Hz, 5.6 Hz, 1H), 2.18 (dd, J = 15.2 Hz, 8.0 Hz, 1H), 1.75-1.65 (m, 4H), 0.96 (d, J = 6.4 Hz, 3H). |
| I-447 | 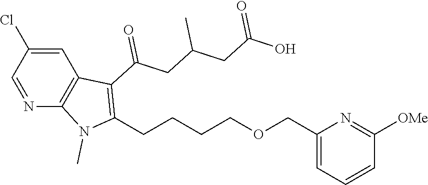 [SMILES: COc1cccc(COCCCCc2c(C(=O)CC(C)CC(=O)O)c3cc(Cl)cnc3n2C)n1] | 488.2 | ¹H NMR (400 MHz, DMSO-d₆) δ 12.10 (br., 1H), 8.38 (d, J = 2.0 Hz, 1H), 8.33 (d, J = 2.0 Hz, 1H), 7.68 (t, J = 8.0 Hz, 1H), 6.99 (d, J = 7.2 Hz, 1H), 6.69 (d, J = 8.0 Hz, 1H), 4.45 (s, 2H), 3.81 (s, 3H), 3.80 (s, 3H), 3.58 (t, J = 5.6 Hz, 2H), 3.24 (t, J = 7.6 Hz, 2H), 3.04 (dd, J = 16.8 Hz, 5.6 Hz, 1H), 2.83 (dd, J = 16.4 Hz, 7.6 Hz, 1H), 2.49-2.44 (m, 1H), 2.39 (dd, J = 15.6 Hz, 5.6 Hz, 1H), 2.17 (dd, J = 15.6 Hz, 8.0 Hz, 1H), 1.78-1.65 (m, 4H), 0.96 (d, J = 6.8 Hz, 3H). |
| I-448 | 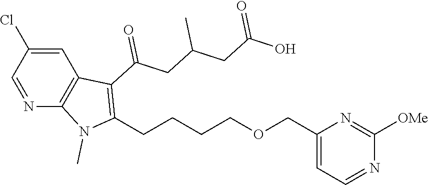 [SMILES: COc1nccc(COCCCCc2c(C(=O)CC(C)CC(=O)O)c3cc(Cl)cnc3n2C)n1] | 489.1 | ¹H NMR (400 MHz, DMSO-d₆) δ 12.07 (s, 1H), 8.54 (d, J = 4.8 Hz, 1H), 8.34 (d, J = 2.4 Hz, 1H), 8.30 (t, J = 2.0 Hz, 1H), 7.10 (d, J = 5.2 Hz, 1H), 4.46 (s, 2H), 3.85 (s, 3H), 3.79 (s, 3H), 3.56 (t, J = 5.6 Hz, 2H), 3.21 (t, J = 7.2 Hz, 2H), 3.01 (dd, J = 16.8 Hz, 5.6 Hz, 1H), 2.84 (dd, J = 16.8 Hz, 7.6 Hz, 1H), 2.47-2.41 (m, 1H), 2.37 (dd, J = 15.6 Hz, 5.6 Hz, 1H), 2.15 (dd, J = 15.6 Hz, 8.0 Hz, 1H), 1.75-1.63 (m, 4H), 0.93 (d, J = 6.8 Hz, 3H). |
| I-449 | 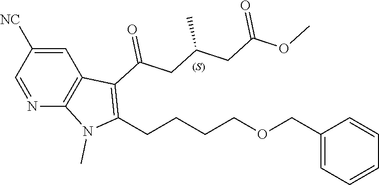 | 462.3 | ¹H NMR (400 MHz, DMSO-d₆) δ 8.81 (d, J = 2.0 Hz, 1H), 8.73 (d, J = 1.6 Hz, 1H), 7.35-7.24 (m, 5H), 4.45 (s, 2H), 3.84 (s, 3H), 3.58 (s, 3H), 3.49 (t, J = 5.6 Hz, 2H), 3.24 (t, J = 6.8 Hz, 2H), 3.08 (dd, J = 16.8 Hz, 5.6 Hz, 1H), 2.95 (dd, J = 16.8 Hz, 6.8 Hz, 1H), 2.56-2.51 (m, 1H), 2.49-2.46 (m, 1H), 2.27 (dd, J = 14.8 Hz, 7.6 Hz, 1H), 1.73-1.63 (m, 4H), 0.96 (d, J = 6.4 Hz, 1H) |
| I-450 | 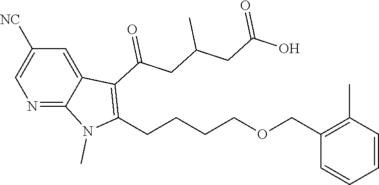 [SMILES: Cc1ccccc1COCCCCc1c(C(=O)CC(C)CC(=O)O)c2cc(C#N)cnc2n1C] | 462.3 | ¹H NMR (400 MHz, DMSO-d₆) δ 12.09 (br., 1H), 8.81 (d, J = 1.6 Hz, 1H), 8.70 (d, J = 2.0 Hz, 1H), 7.26-7.24 (m, 1H), 7.14-7.09 (m, 3H), 4.41 (s, 2H), 3.81 (s, 3H), 3.48 (t, J = 5.6 Hz, 2H), 3.22 (t, J = 6.8 Hz, 2H). 3.07 (dd, J = 17.2 Hz, 5.6 Hz, 1H), 2.88 (dd, J = 16.8 Hz, 7.6 Hz, 1H), 2.49-2.42 (m, 1H), 2.37 (dd, J = 15.6 Hz, 6.0 Hz, 1H), 2.21 (s, 3H), 2.14 (dd, J = 15.2 Hz, 8.0 Hz, 1H), 1.71-1.59 (m, 4H), 0.93 (d, J = 6.4 Hz, 3H). |
| I-451 | 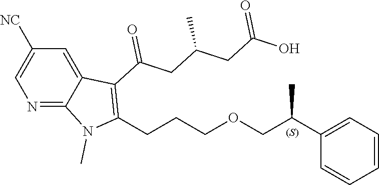 | 462.3 | ¹H NMR (400 MHz, DMSO-d₆) δ 12.08 (s, 1H), 8.84 (d, J = 1.6 Hz, 1H), 8.74 (d, J = 2.0 Hz, 1H), 7.30-7.16 (m, 5H), 3.77 (s, 3H), 3.49-3.44 (m, 4H), 3.20 (t, J = 7.2 Hz, 2H), 3.09 (dd, J = 16.8 Hz, 5.2 Hz, 1H), 2.96-2.87 (m, 2H), 2.49-2.45 (m, 1H), 2.41 (dd, J = 15.6 Hz, 5.6 Hz, 1H), 2.18 (dd, J = 15.6 Hz, 8.0 Hz, 1H), 1.88- 1.77 (m, 2H), 1.18 (d, J = 6.8 Hz, 3H), 0.96 (d, J = 6.8 Hz, 3H). |
| I-452 | 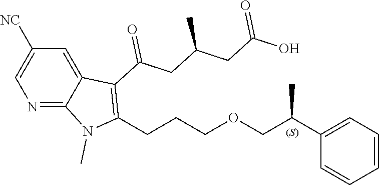 | 462.3 | ¹H NMR (400 MHz, DMSO-d₆) δ 12.08 (s, 1H), 8.84 (d, J = 2.0 Hz, 1H), 8.74 (d, J = 2.0 Hz, 1H), 7.30-7.16 (m, 5H), 3.77 (s, 3H), 3.51-3.41 (m, 4H), 3.20 (t, J = 7.6 Hz, 2H), 3.09 (dd, J = 16.8 Hz, 6.4 Hz, 1H), 2.96-2.87 (m, 2H), 2.49-2.45 (m, 1H), 2.41 (dd, J = 15.6 Hz, 5.6 Hz, 1H), 2.18 (dd, J = 15.6 Hz, 8.0 Hz, 1H), 1.88- 1.77 (m, 2H), 1.18 (d, J = 7.2 Hz, 3H), 0.96 (d, J = 6.4 Hz, 3H). |
| I-453 | 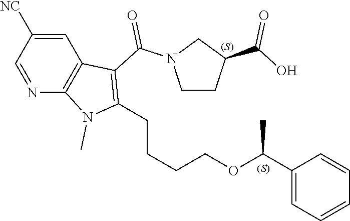 | 475.3 | ¹H NMR (400 MHz, DMSO-d₆) δ 12.59 (br., 1H), 8.63 (d, J = 2.0 Hz, 1H), 8.35 (s, 1H), 7.31-7.19 (m, 5H), 4.35 (q, J = 6.4 Hz, 1H), 3.77 (s, 3H), 3.70-3.41 (m, 3H), 3.28-3.11 (m, 3H), 3.09-2.94 (m, 1H), 2.89 (t, J = 7.2 Hz, 2H), 2.05 (br., 2H), 1.70-1.51 (m, 2H), 1.52-1.41 (m, 2H), 1.27 (d, J = 6.4 Hz, 3H). |
| I-454 | 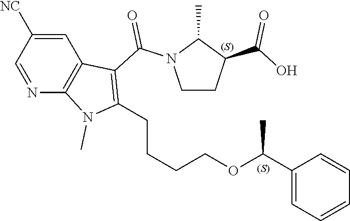 | 489.3 | ¹H NMR (400 MHz, DMSO-d₆) δ 12.62 (br., 1H), 8.65 (d, J = 1.6 Hz, 1H), 8.33 (s, 1H), 7.33-7.21 (m, 5H), 4.37 (q, J = 6.4 Hz, 1H), 4.30 (s, 1H), 3.81 (s, 3H), 3.48 (br., 1H), 3.30-3.11 (m, 3H), 3.04-2.95 (m, 1H), 2.93-2.81 (m, 1H), 2.79-2.71 (m, 1H), 2.07 (br., 1H), 1.84 (br., 1H), 1.73- 1.64 (m, 1H), 1.62-1.45 (m, 3H), 1.40- 1.02 (m, 6H). |
| I-455 | 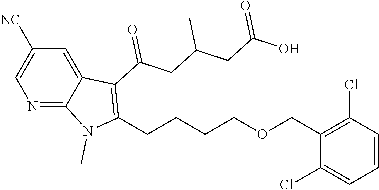 [SMILES: CC(CC(=O)O)CC(=O)c1c(CCCCOCc2c(Cl)cccc2Cl)n(C)c2ncc(C#N)cc12] | 516.0 | ¹H NMR (400 MHz, DMSO-d₆) δ 12.09 (s, 1H), 8.83 (d, J = 2.0 Hz, 1H), 8.73 (d, J = 2.0 Hz, 1H), 7.48 (d, J = 1.2 Hz, 1H), 7.46 (s, 1H), 7.38-7.34 (m, 1H), 4.65 (s, 2H), 3.83 (s, 3H), 3.55 (t, J = 5.2 Hz, 2H), 3.23 (t, J = 6.8 Hz, 2H), 3.08 (dd, J = 16.8 Hz, 5.6 Hz, 1H), 2.90 (dd, J = 16.8 Hz, 8.0 Hz, 1H), 2.55-2.51 (m, 1H), 2.40 (dd, J = 15.6 Hz, 6.0 Hz, 1H), 2.17 (dd, J = 15.2 Hz, 7.6 Hz, 1H), 1.67-1.66 (m, 4H), 0.95 (d, J = 6.4 Hz, 3H). |

TABLE 12a
Additional Characterization Data for Additional Exemplary Compounds

| | Rt (Min) | Rt (Min) |
|---|---|---|
| Compound No. | (LCMS) | (HPLC) |
| I-365 | 1.894 | 9.414 |
| I-366 | 1.896 | 9.423 |
| I-367 | 1.849 | 10.201 |
| I-368 | 1.689 | 8.801 |
| I-369 | 1.688 | 8.799 |
| I-370 | 1.667 | 7.914 |
| I-371 | 1.690 | 8.034 |
| I-372 | 1.689 | 8.058 |
| I-373 | 1.703 | 8.096 |
| I-374 | 1.663 | 7.830 |
| I-375 | 1.625 | 7.539 |
| I-376 | 1.814 | 8.887 |
| I-377 | 1.825 | 8.934 |
| I-378 | 1.920 | 9.521 |
| I-379 | 2.001 | 10.061 |
| I-380 | 2.063 | 10.438 |
| I-381 | 2.035 | 10.250 |
| I-382 | 1.847 | 9.178 |
| I-383 | 1.805 | 8.829 |
| I-384 | 1.805 | 8.827 |
| I-385 | 2.074 | 10.621 |
| I-386 | 2.074 | 10.618 |
| I-387 | 1.926 | 9.569 |
| I-388 | 1.541 | 7.074 |
| I-389 | 2.004 | 10.120 |
| I-390 | 2.004 | 10.133 |
| I-391 | 1.862 | 9.131 |
| I-392 | 1.744 | 8.365 |
| I-393 | 1.560 | 7.206 |
| I-394 | 1.499 | 6.588 |
| I-395 | 1.496 | 6.577 |
| I-396 | 1.135 | 6.618 |
| I-397 | 1.135 | 6.633 |
| I-398 | 1.461 | 6.484 |
| I-399 | 2.106 | 10.728 |
| I-400 | 2.105 | 10.785 |
| I-401 | 2.107 | 10.802 |
| I-402 | 2.047 | 10.461 |
| I-403 | 1.991 | 9.994 |
| I-404 | 1.742 | 8.411 |
| I-405 | 1.742 | 8.411 |
| I-406 | 1.655 | 7.329 |
| I-407 | 1.654 | 7.322 |
| I-408 | 1.705 | 7.809 |
| I-409 | 1.702 | 7.804 |
| I-410 | 1.708 | 9.340 |
| I-411 | 1.707 | 9.337 |
| I-412 | 1.934 | 9.655 |
| I-413 | 1.893 | 9.376 |
| I-414 | 1.893 | 9.378 |
| I-415 | 2.019 | 10.281 |
| I-416 | 1.841 | 9.114 |
| I-417 | 1.952 | 9.810 |
| I-418 | 1.967 | 9.930 |
| I-419 | 2.010 | 10.115 |
| I-420 | 1.705 | 9.303 |
| I-421 | 1.958 | 9.854 |
| I-422 | 1.922 | 9.698 |
| I-423 | 1.924 | 9.708 |
| I-424 | 1.928 | 9.629 |
| I-425 | 1.999 | 10.131 |
| I-426 | 1.735 | 8.198 |
| I-427 | 1.611 | 7.579 |
| I-428 | 1.678 | 8.055 |
| I-429 | 1.451 | 5.945 |
| I-430 | 2.081 | 10.552 |
| I-431 | 1.947 | 9.734 |
| I-432 | 1.947 | 9.733 |
| I-433 | 1.947 | 9.735 |
| I-434 | 1.947 | 9.732 |
| I-435 | 1.928 | 9.650 |
| I-436 | 1.990 | 10.026 |
| I-437 | 1.989 | 10.032 |
| I-438 | 1.944 | 9.730 |
| I-439 | 1.925 | 9.724 |
| I-440 | 1.957 | 9.803 |
| I-441 | 1.421 | 5.762 |
| I-442 | 1.522 | 6.812 |
| I-443 | 1.920 | 9.551 |
| I-444 | 1.823 | 8.923 |
| I-445 | 1.798 | 8.744 |
| I-446 | 1.772 | 8.443 |
| I-447 | 1.909 | 9.442 |
| I-448 | 1.794 | 8.724 |
| I-449 | 2.071 | 10.539 |
| I-450 | 1.956 | 9.792 |
| I-451 | 1.977 | 9.932 |
| I-452 | 1.977 | 9.929 |
| I-453 | 1.730 | 8.323 |
| I-454 | 1.794 | 8.739 |
| I-455 | 2.012 | 10.145 |

### Example 12: Synthesis of compounds I-456 and I-457

### Synthetic Scheme for Compounds I-456 and I-457

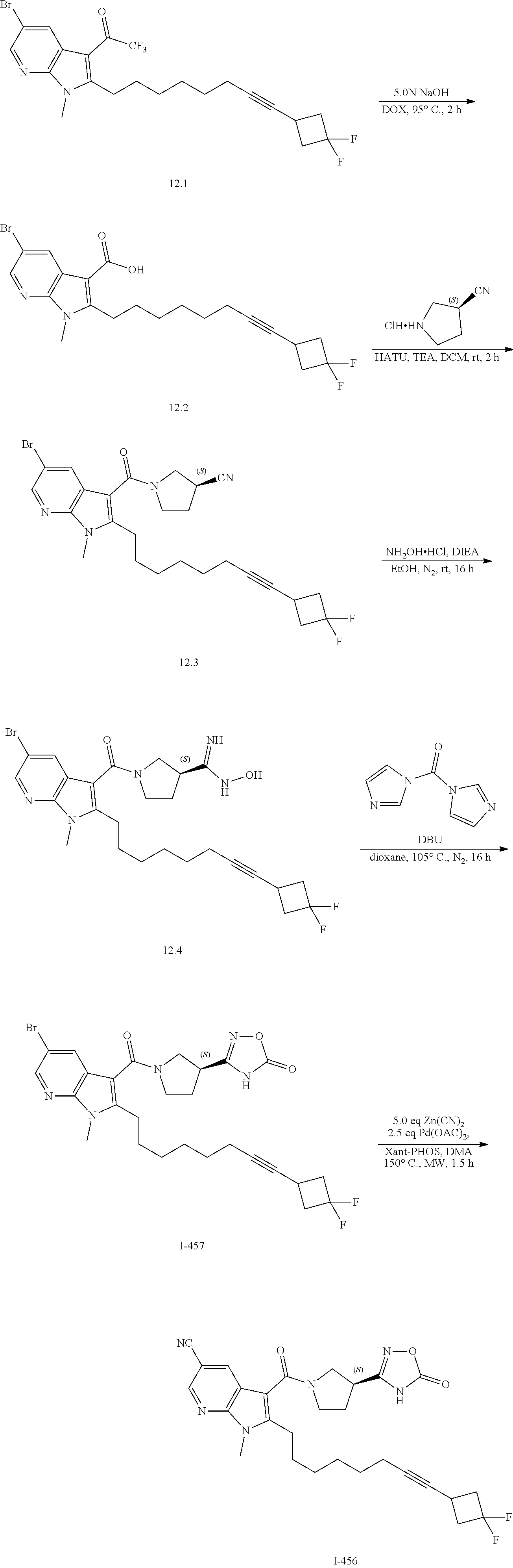

### 12.1 The Synthesis of Intermediate 12-2

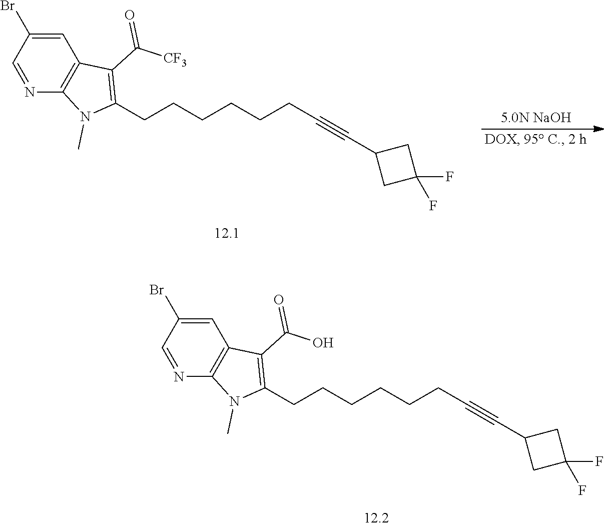 [SMILES: Cn1c(CCCCCCC#CC2CC(F)(F)C2)c(C(=O)C(F)(F)F)c2cc(Br)cnc21 ; "5.0N NaOH" ; "DOX, 95° C., 2 h" ; Cn1c(CCCCCCC#CC2CC(F)(F)C2)c(C(=O)O)c2cc(Br)cnc21]

To a solution of 12-1 (2.1 g, 0.5 mmol) in dioxane (36 mL) and EtOH (4 mL) was added NaOH (5.0 M, 30 mL) at 0° C. Then the reaction mixture was heated to 95° C. for 2 h. The mixture was acidified with HCl (3.0 M) to pH ˜3.0 then extracted with EtOAc (50 mL×3), the combined organic layer was washed with brine, dried over anhydrous Na₂SO₄ and evaporated under reduced pressure. The crude was purified by silica gel chromatography (MeOH/DCM: 13%) to afford 12-2 (1.2 g, yield: 64%) as a yellow solid.

### 12.2 The Synthesis of Intermediate 12-3

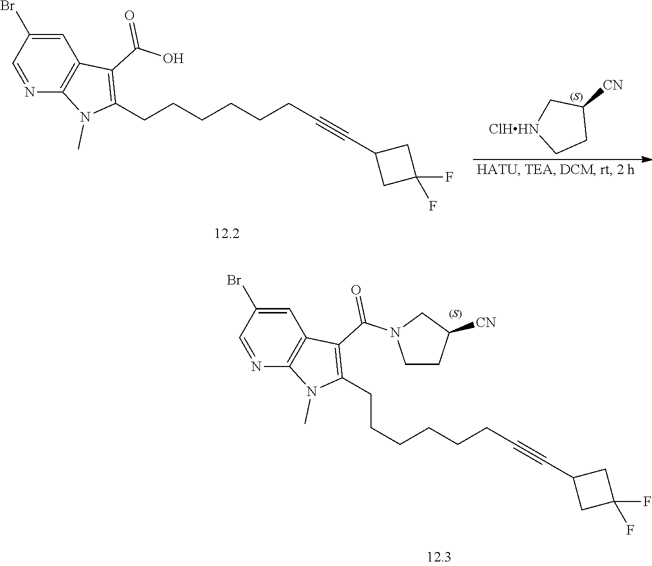

To a solution of 12-2 (270 mg, 0.60 mmol) in DMF (3 mL) was added HATU (250 mg, 0.66 mmol) and TEA (181 mg, 1.8 mmol) at 0° C. The mixture was stirred for 15 min, then (S)-pyrrolidine-3-carbonitrile hydrochloride (87 mg, 0.66 mmol) was added. The reaction mixture was allowed to warm to room temperature and stirred for 2 h. The reaction was quenched by adding water (50 mL) and extracted with EtOAc (50 mL×3). The organic layer was combined, washed with brine and dried over Na₂SO₄. The solvent was evaporated under reduced pressure and the crude was purified by silica gel chromatography (EtOAc/PE: 50%) to afford 12-3 (283 mg, yield: 86.5%) as a yellow oil.

### 12.3 The Synthesis of Intermediate 12-4

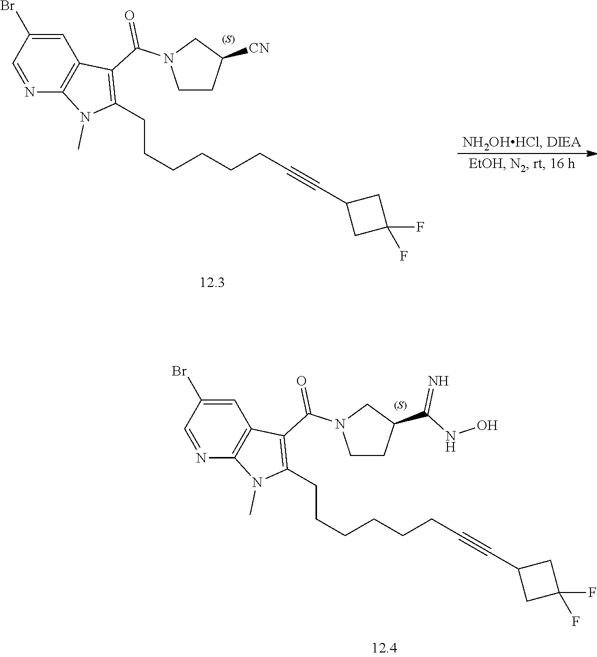

To a stirred solution of 12-3 (230 mg, 0.44 mmol) in EtOH (10 mL) was added NH₂OH·HCl (62 mg, 0.89 mmol) and DIEA (57 mg, 0.44 mmol). The reaction mixture was stirred at room temperature for 16 h under nitrogen. The reaction was quenched by adding water (30 mL) and extracted with EtOAc (30 mL×3). The organic layer was combined, washed with brine and dried over Na₂SO₄. The mixture was evaporated under reduced pressure then purified by silica gel chromatograph column (PE/EA: 70%) to give 12-4 (151 mg, yield: 60%) as a yellow solid. 12.4 The Synthesis of Compound I-457

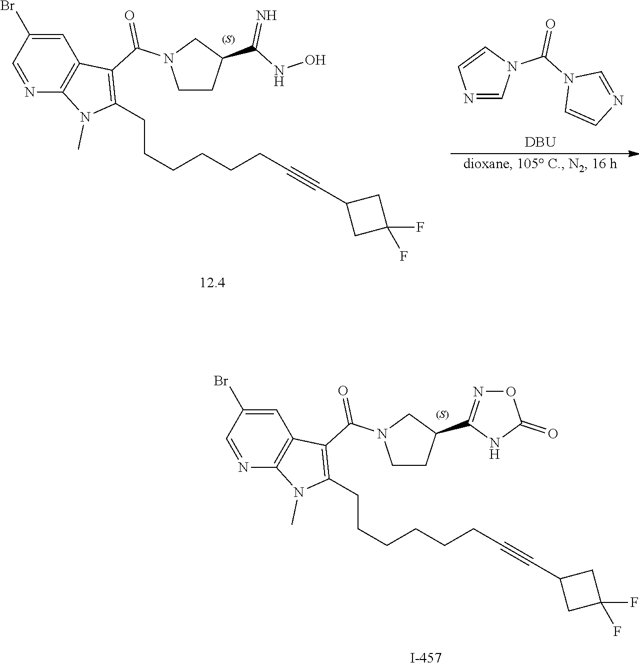

To a mixture of DBU (53 mg, 0.34 mmol) in dioxane (3 mL) was added CDI (60 mg, 0.37 mmol), the reaction mixture was added 12-4 (140 mg, 0.25 mmol). The reaction mixture was stirred at 105° C. for 16 h under nitrogen until the reaction was complete (by LCMS). The reaction was quenched by adding water (30 mL) and extracted with EtOAc (30 mL×3). The organic layer was combined, washed with brine and dried over Na₂SO₄ and evaporated under reduced pressure. The mixture was evaporated under reduced pressure then purified by silica gel chromatograph column (PE/EA: 40%) to afford 1-457 (85 mg, 58%) as a yellow oil. ¹H NMR (400 MHz, DMSO-d₆) δ 12.32 (br., 1H), 8.32 (d, J=2.4 Hz, 1H), 8.06 (d, J=2.4 Hz, 1H), 3.77 (s, 3H), 3.60-3.33 (m, 5H), 2.96-2.82 (m, 5H), 2.57-2.51 (m, 1H), 2.48-2.42 (m, 1H), 2.30-2.05 (m, 4H), 1.63-1.51 (m, 2H), 1.44-1.27 (m, 6H). LC-MS m/z: 590.0 & 592.0 [(⁷⁹Br & ⁸¹Br)M+H]⁺. HPLC Purity (214 nm): >99.9%; t_{R}=9.685 min.

### 12.5 The Synthesis of Compound I-456

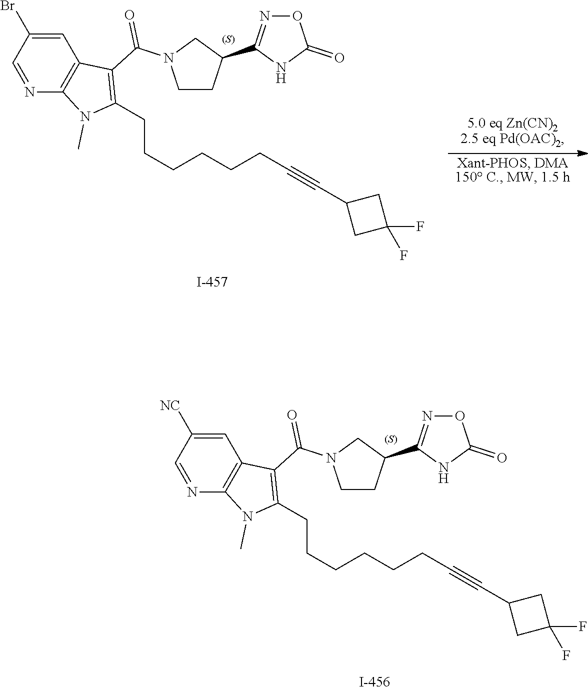

To a mixture of 1-457 (52 mg, 0.09 mmol) in DMA (2 mL) was added Zn(CN)₂ (52 mg, 0.44 mmol) and Xant-PHOS (41 mg, 0.07 mmol) and Pd(OAC)₂ (44 mg, 0.19 mmol), the reaction mixture was stirred at 150° C. for 1.2 h until the reaction was complete (by LCMS). The reaction was quenched by adding water (30 mL) and extracted with EtOAc (30 mL×3). The organic layer was combined, washed with brine and dried over Na₂SO₄ and evaporated under reduced pressure. The crude was purified by Prep-HPLC to afford 1-456 (1.20 mg, 3%) as a white solid. ¹H NMR (400 MHz, DMSO-d₆) δ 12.33 (s, 1H), 8.66 (d, J=1.6 Hz, 1H), 8.41 (d, J=1.6 Hz, 1H), 3.83 (s, 3H), 3.76-3.33 (m, 5H), 3.02-2.81 (m, 5H), 2.56-2.51 (m, 2H), 2.28-2.17 (m, 1H), 2.15 (t, J=5.6 Hz, 3H), 1.64-1.53 (m, 2H), 1.44-1.27 (m, 6H). LC-MS m/z: 537.2 [M+H]⁺. HPLC Purity (214 nm): 98.65%; t_{R}^{=9.108} min.

### Example 13: Synthesis of Additional Compounds

The following compounds were prepared using procedures based on those described herein above.

| Compound | | | |
|---|---|---|---|
| No. | Chemical Structure | M + 1 | ¹H NMR (400 MHz) |
| I-458 | 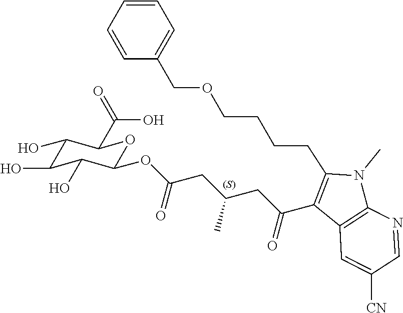 | 646.2 | ¹H NMR (400 MHz, DMSO-d₆) δ 8.82 (d, J = 2.0 Hz, 1H), 8.73 (d, J = 2.0 Hz, 1H), 7.35-7.25 (m, 5H), 5.38 (d, J = 8.4 Hz, 1H), 5.27 (s, 1H), 5.13 (s, 1H), 4.46 (s, 2H), 3.84 (s, 3H), 3.52-3.47 (m, 3H), 3.27-3.06 (m, 7H), 2.98 (dd, J = 17.2 Hz, 7.2 Hz, 1H), 2.65-2.52 (m, 2H), 2.32 (dd, J = 15.2 Hz, 8.0 Hz, 1H), 1.75-1.62 (m, 4H), 1.00 (d, J = 6.4 Hz, 3H). |
| I-459 | 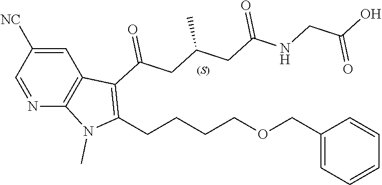 | 505.1 | (DMSO-d₆) δ 12.48 (br., 1H), 8.79 (d, J = 2.0 Hz, 1H), 8.73 (d, J = 2.0 Hz, 1H), 8.24, (t, J = 6.0 Hz, 1H), 7.35-7.24 (m, 5H), 4.45 (s, 2H), 3.84 (s, 3H), 3.80-3.68 (m, 2H), 3.49, (t, J = 5.6 Hz, 2H), 3.24 (t, J = 7.2 Hz, 2H), 3.17 (dd, J = 16.8 Hz, 4.8 Hz, 1H), 2.83 (dd, J = 16.8 Hz, 8.8 Hz, 1H), 2.49-2.38 (m, 1H), 2.25 (dd, J = 13.6 Hz, 6.8 Hz, 1H), 2.13 (dd, J = 13.6 Hz, 7.2 Hz, 1H), 1.74-1.60 (m, 4H), 0.94 (d, J = 6.8 Hz, 3H). |
| I-460 | 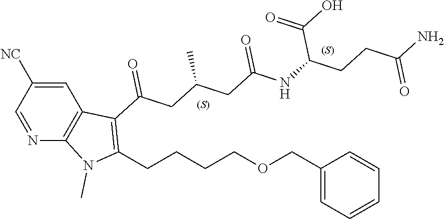 | 576.3 | (DMSO-d₆) δ 12.50 (s, 1H), 8.80 (d, J = 1.6 Hz, 1H), 8.73 (d, J = 1.6 Hz, 1H), 8.18 (d, J = 7.6 Hz, 1H), 7.35-7.26 (m, 6H), 6.76 (s, 1H), 4.46 (s, 2H), 4.17-4.14 (m, 1H), 3.84 (s, 3H), 3.50 (t, J = 10.8 Hz, 2H), 3.26-3.22 (m, 2H), 3.11 (dd, J = 16.8 Hz, 5.2 Hz, 1H), 2.88 (dd, J = 16.8 Hz, 8.4 Hz, 1H), 2.50-2.44 (m, 1H), 2.25 (dd, J = 14.0 Hz, 6.4 Hz, 1H), 2.15-2.11 (m, 3H), 1.99-1.89 (m, 1H), 1.82-1.63 (m, 5H), 0.94 (d, J = 6.8 Hz, 3H). |
| I-461 | 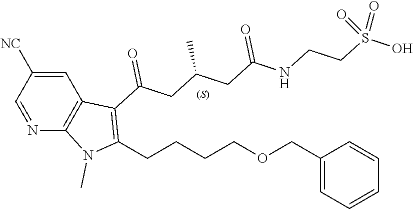 | 555.3 | (DMSO-d₆) δ 8.78 (d, J = 2.0 Hz, 1H), 8.69 (d, J = 2.0 Hz, 1H), 7.73 (t, J = 5.6 Hz, 1H). 7.33-7.20 (m, 5H), 7.06 (s, 4H), 4.43 (s, 2H), 3.81 (s, 3H), 3.47 (t, J = 5.6 Hz, 2H), 3.29-3.25 (m, 2H), 3.21 (t, J = 7.2 Hz, 2H). 3.04 (dd, J = 16.4 Hz, 5.2 Hz, 1H), 2.82 (dd, J = 16.4 Hz, 8.0 Hz, 1H), 2.52-2.51 (m, 2H), 2.48-2.46 (m, 1H), 2.17 (dd, J = 13.6 Hz, 6.0 Hz, 1H), 1.98 (dd, J = 14.0 Hz, 8.0 Hz, 1H), 1.71- 1.58 (m, 4H), 0.88 (d, J = 6.4 Hz, 3H). |
| I-462 | 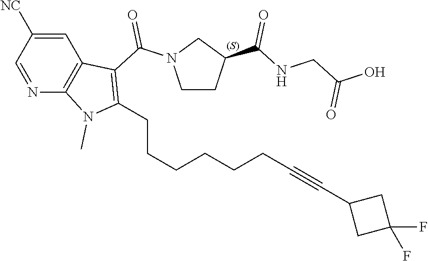 | 554.2 | (DMSO-d₆) δ 12.56 (br., 1H), 8.65 (d, J = 2.0 Hz, 1H), 8.44-8.16 (m, 2H), 3.83 (s, 3H), 3.74 (s, 2H), 3.66-3.34 (m, 4H), 3.15-2.86 (m, 6H), 2.57-2.51 (m, 2H), 2.17-1.86 (m, 4H), 1.66-1.53 (m, 2H), 1.43-1.22 (m, 6H). |
| I-463 | 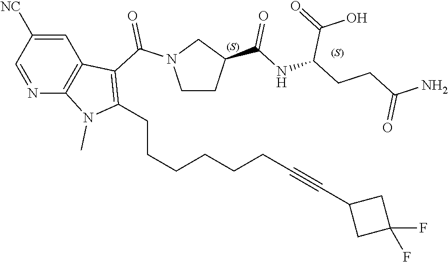 | 625.3 | (DMSO-d₆) δ 12.55 (br., 1H), 8.65 (d, J = 1.6 Hz, 1H), 8.39 (s, 1H), 8.20 (br., 1H), 7.29 (s, 1H), 6.78 (s, 1H), 4.15 (s, 1H), 3.83 (s, 3H), 3.76-3.34 (m, 4H), 3.16-2.86 (m, 6H), 2.56-2.51 (m, 2H), 2.19-2.09 (m, 4H), 2.09-1.92 (m, 3H), 1.76-1.74 (m, 1H), 1.66-1.55 (m, 2H), 1.45-1.25 (m, 6H). |
| I-464 | 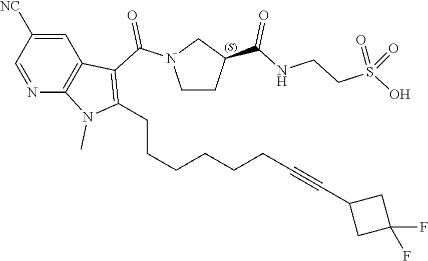 | 604.2 | (DMSO-d₆) δ 8.64 (d, J = 2.0 Hz, 1H), 8.40 (s, 1H), 7.86 (br., 1H), 4.07 (s, 5H), 3.83 (s, 3H), 3.67-3.38 (m, 3H), 3.15- 3.04 (m, 1H), 2.98-2.88 (m, 5H), 2.59- 2.52 (m, 2H), 2.16-1.85 (m, 4H), 1.66- 1.54 (m, 2H), 1.46-1.26 (m, 6H), 1.18 (t, J = 7.2 Hz, 1H). |
| I-465 | 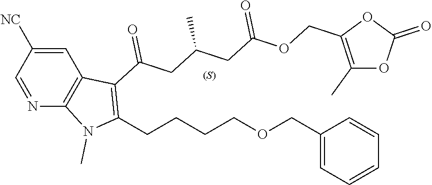 | 560.5 | (DMSO-d₆) δ 8.81 (d, J = 2.0 Hz, 1H), 8.74 (d, J = 1.6 Hz, 1H), 7.35-7.26 (m, 5H), 4.95 (s, 2H), 4.45 (s, 2H), 3.84 (s, 3H), 3.49 (t, J = 5.6 Hz, 2H), 3.24 (t, J = 6.8 Hz, 2H), 3.09 (dd, J = 17.2 Hz, 5.6 Hz, 1H), 2.97 (dd, J = 17.2 Hz, 6.8 Hz, 1H), 2.57-2.53 (m, 1H), 2.49-2.44 (m, 1H), 2.36-2.29 (m, 1H), 2.12 (s, 3H), 1.73-1.60 (m, 4H), 0.97 (d, J = 6.4 Hz, 3H). |
| I-466 | 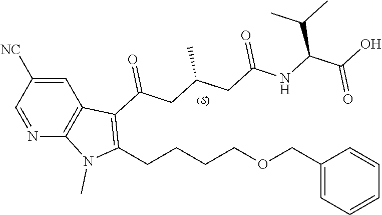 | 547.3 | (DMSO-d₆) δ 12.38 (br., 1H), 8.76 (d, J = 2.0 Hz, 1H), 8.71 (d, J = 1.6 Hz, 1H), 8.02 (d, J = 8.4 Hz, 1H), 7.32-7.24 (m, 5H), 4.43 (s, 2H), 4.14 (dd, J = 8.4 Hz, 5.6 Hz, 1H), 3.81 (s, 3H), 3.46 (t, J = 5.6 Hz, 2H), 3.22 (t, J = 7.2 Hz, 2H), 3.07 (dd, J = 16.8 Hz, 4.4 Hz, 1H), 2.84 (dd, J = 16.8 Hz, 8.8 Hz, 1H), 2.47-2.45 (m, 1H), 2.29 (dd, J = 13.6 Hz, 6.8 Hz, 1H), 2.16 (dd, J = 13.6 Hz, 6.8 Hz, 1H), 2.06- 1.98 (m, 2H), 1.67-1.65 (m, 4H), 0.92 (d, J = 6.8 Hz, 3H), 0.85 (d, J = 6.8 Hz, 3H), 0.82 (d, J = 6.8 Hz, 3H). |
| I-467 | 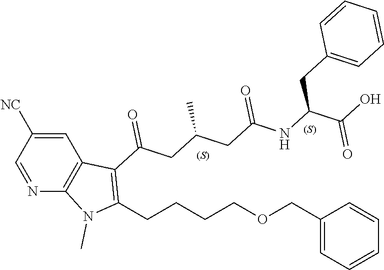 | 595.3 | (DMSO-d₆) δ 12.66 (s, 1H), 8.70 (s, 1H), 8.60 (d, J = 1.2 Hz, 1H), 8.25 (d, J = 8.4 Hz, 1H), 7.32-7.17 (m, 5H), 7.18 (d, J = 7.6 Hz, 2H), 7.07 (t, J = 7.2 Hz, 2H), 6.94 (t, J = 7.2 Hz, 1H), 4.52-4.46 (m, 1H), 4.43 (s, 2H), 3.81 (s, 3H), 3.46 (t, J = 5.6 Hz, 2H), 3.20-3.17 (m, 2H), 3.06 (dd, J = 13.6 Hz, 4.0 Hz, 1H), 2.85-2.78 (m, 2H), 2.64 (dd, J = 16.4 Hz, 8.8 Hz, 1H), 2.39- 2.31 (m, 1H), 2.12 (dd, J = 13.6 Hz, 7.2 Hz, 1H), 2.04 (dd, J = 13.6 Hz, 6.8 Hz, 1H), 1.65-1.61 (m, 4H), 0.80 (d, J = 6.8 Hz, 3H). |
| I-469 | 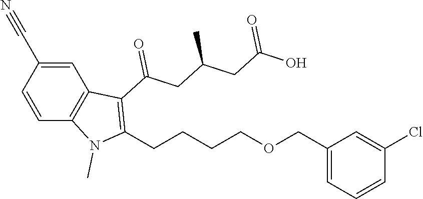 [SMILES: C[C@@H](CC(=O)O)CC(=O)c1c(CCCCOCc2cccc(Cl)c2)n(C)c2ccc(C#N)cc12] | 481.2 | (DMSO-d₆) δ 12.09 (s, 1H), 8.43 (s, 1H), 7.77 (d, J = 8.8 Hz, 1H), 7.62 (dd, J = 8.4 Hz, 1.2 Hz, 1H), 7.39-7.27 (m, 4H), 4.47 (s, 2H), 3.81 (s, 3H), 3.50 (t, J = 6.0 Hz, 2H), 3.20 (t, J = 7.6 Hz, 2H), 3.07 (dd, J = 16.4 Hz, 5.6 Hz, 1H), 2.91 (dd, J = 16.8 Hz, 7.6 Hz, 1H), 2.51 (s, 1H), 2.41 (dd, J = 15.2 Hz, 5.6 Hz, 1H), 2.18 (dd, J = 15.2 Hz, 8.0 Hz, 1H), 1.72- 1.65 (m, 4H), 0.96 (d, J = 6.8 Hz, 3H). |

Table of Additional Characterization Data for Additional
Exemplary Compounds

| | Rt (Min) | Rt (Min) |
|---|---|---|
| Compound No. | (LCMS) | (HPLC) |
| I-458 | 1.700 | 8.158 |
| I-459 | 1.755 | 8.488 |
| I-460 | 1.666 | 7.915 |
| I-461 | 1.569 | 7.328 |
| I-462 | 1.736 | 8.407 |
| I-463 | 1.665 | 7.923 |
| I-464 | 1.578 | 7.488 |
| I-465 | 2.370 | N/A |
| I-466 | 1.885 | 9.314 |
| I-467 | 1.925 | 9.583 |
| I-469 | 1.959 | 9.845 |

### Example 14: Neutrophil Calcium Flux Assay

After isolation, neutrophils were resuspended in PBS without calcium and Magnesium (PBS −/−) at a concentration of 5×106 cells/ml. 2ul of Indo-1 (1 mM) were added per ml of cells. After incubation at 37° C. for 30 minutes, cells were washed twice in PBS −/− and resuspended at 5×106 cells/ml in assay buffer (HBSS 1× with calcium and magnesium and 1M HEPES). In a 96-well Black optical clear bottom plate 100 μL of neutrophil were dispensed to each well. After incubation at 37° C. for 5 minutes, 50 μL of assay buffer or 3× antagonist were added and incubated for 5 min in a Tecan Spark plate reader (Tecan, Morrisville, NC). Baseline was measured for 30 sec (wavelength setup: excitation: 331 nm and emission:410 nm). When baseline is stable, 50p of 5-OXO-ETE at 200 nM (4×) or HBSS was added to appropriate wells. Fluorescence was measured every 5 seconds for 30 cycles. Results are expressed as Net fluorescence (FLU minus Background and injection peak). All data were analyzed using GraphPad Prism version 9 for Windows (GraphPad, San Diego, CA).

Results: Results are provided in Table 13 below for exemplary compounds. The symbol “***” indicates a IC₅₀ less than 100 nM. The symbol “**” indicates an IC₅₀ in the range of 100 nM to 300 nM. The symbol “*” indicates a IC₅₀ greater than 300 nM.

TABLE 13

| Compound No. | IC₅₀ |
|---|---|
| I-1 | * |
| I-2 | *** |
| I-3 | * |
| I-4 | * |
| I-5 | * |
| I-6 | * |
| I-7 | *** |
| I-8 | * |
| I-9 | *** |
| I-10 | *** |
| I-11 | ** |
| I-12 | * |
| I-13 | *** |
| I-14 | ** |
| I-15 | * |
| I-17 | * |
| I-18 | * |
| I-19 | * |
| I-20 | ** |
| I-21 | * |
| I-22 | * |
| I-23 | ** |
| I-24 | * |
| I-39 | * |
| I-40 | *** |
| I-44 | *** |
| I-45 | ** |
| I-47 | *** |
| I-48 | *** |
| I-53 | *** |
| I-54 | ** |
| I-55 | *** |
| I-58 | *** |
| I-59 | *** |
| I-60 | *** |
| I-61 | *** |
| I-62 | *** |
| I-63 | *** |
| I-64 | ** |
| I-65 | ** |
| I-68 | *** |
| I-70 | ** |
| I-71 | * |
| I-72 | * |
| I-73 | * |
| I-74 | * |
| I-79 | *** |
| I-80 | * |
| I-81 | ** |
| I-82 | ** |
| I-83 | *** |
| I-84 | *** |
| I-85 | *** |
| I-96 | *** |
| I-97 | *** |
| I-98 | *** |
| I-99 | ** |
| I-100 | ** |
| I-101 | * |
| I-103 | * |
| I-104 | *** |
| I-105 | *** |
| I-106 | * |
| I-108 | ** |
| I-111 | ** |
| I-112 | *** |
| I-113 | * |
| I-114 | * |
| I-116 | *** |
| I-117 | *** |
| I-118 | *** |
| I-119 | * |
| I-120 | ** |
| I-121 | * |
| I-124 | ** |
| I-126 | * |
| I-127 | * |
| I-128 | * |
| I-129 | * |
| I-130 | * |
| I-131 | * |
| I-132 | * |
| I-134 | * |
| I-137 | * |
| I-139 | * |
| I-140 | * |
| I-141 | * |
| I-142 | *** |
| I-144 | *** |
| I-145 | * |
| I-146 | ** |
| I-147 | * |
| I-148 | * |
| I-149 | *** |
| I-150 | * |
| I-151 | ** |
| I-152 | * |
| I-153 | * |
| I-154 | * |
| I-155 | ** |
| I-156 | * |
| I-157 | * |
| I-162 | * |
| I-166 | * |
| I-167 | * |
| I-171 | * |
| I-172 | * |
| I-173 | * |
| I-174 | * |
| I-175 | * |
| I-176 | *** |
| I-178 | * |
| I-179 | * |
| I-180 | *** |
| I-181 | * |
| I-182 | *** |
| I-183 | * |
| I-184 | * |
| I-185 | *** |
| I-187 | ** |
| I-188 | ** |
| I-189 | ** |
| I-191 | * |
| I-192 | * |
| I-284 | *** |
| I-285 | *** |
| I-212 | *** |
| I-213 | * |
| I-286 | *** |
| I-215 | * |
| I-216 | * |
| I-218 | * |
| I-220 | * |
| I-223 | *** |
| I-224 | ** |
| I-225 | ** |
| I-227 | * |
| I-228 | * |
| I-229 | ** |
| I-231 | *** |
| I-292 | * |
| I-237 | * |
| I-238 | * |
| I-239 | * |
| I-240 | * |
| I-241 | * |
| I-242 | * |
| I-243 | * |
| I-278 | *** |
| I-279 | *** |
| I-280 | * |
| I-281 | * |
| I-282 | * |
| I-283 | ** |
| I-244 | * |
| I-287 | * |
| I-288 | ** |
| I-246 | ** |
| I-289 | ** |
| I-290 | ** |
| I-249 | *** |
| I-291 | *** |
| I-293 | *** |
| I-294 | * |
| I-295 | * |
| I-296 | ** |
| I-297 | *** |
| I-298 | ** |
| I-299 | ** |
| I-300 | * |
| I-301 | *** |
| I-302 | *** |
| I-303 | * |
| I-304 | * |
| I-110 | *** |
| I-107 | *** |
| I-305 | *** |
| I-306 | * |
| I-307 | *** |
| I-308 | *** |
| I-309 | * |
| I-310 | ** |
| I-311 | * |
| I-312 | * |
| I-313 | * |
| I-314 | *** |
| I-315 | * |
| I-316 | * |
| I-317 | *** |
| I-318 | ** |
| I-319 | *** |
| I-320 | *** |
| I-321 | *** |
| I-322 | *** |
| I-323 | *** |
| I-324 | *** |
| I-325 | *** |
| I-326 | *** |
| I-327 | *** |
| I-328 | *** |
| I-329 | * |
| I-330 | * |
| I-331 | *** |
| I-332 | *** |
| I-333 | * |
| I-334 | * |
| I-335 | *** |
| I-336 | * |
| I-337 | *** |
| I-338 | *** |
| I-339 | *** |
| I-340 | *** |
| I-341 | * |
| I-342 | * |
| I-343 | * |
| I-344 | * |
| I-345 | ** |
| I-346 | *** |
| I-347 | * |
| I-348 | * |
| I-349 | * |
| I-350 | * |
| I-351 | * |
| I-352 | *** |
| I-353 | *** |
| I-354 | *** |
| I-355 | *** |
| I-356 | * |
| I-357 | * |
| I-358 | * |
| I-359 | *** |
| I-360 | * |
| I-361 | * |
| I-362 | * |
| I-363 | * |
| I-364 | * |
| I-366 | ** |
| I-367 | * |
| I-368 | ** |
| I-369 | * |
| I-370 | *** |
| I-371 | *** |
| I-372 | * |
| I-373 | ** |
| I-374 | ** |
| I-375 | * |
| I-376 | * |
| I-377 | * |
| I-378 | * |
| I-379 | * |
| I-380 | * |
| I-381 | * |
| I-382 | *** |
| I-383 | *** |
| I-384 | * |
| I-385 | *** |
| I-386 | ** |
| I-387 | ** |
| I-388 | * |
| I-389 | *** |
| I-390 | * |
| I-391 | *** |
| I-392 | ** |
| I-393 | * |
| I-394 | *** |
| I-395 | * |
| I-396 | *** |
| I-397 | * |
| I-398 | * |
| I-399 | * |
| I-400 | * |
| I-401 | * |
| I-402 | ** |
| I-403 | ** |
| I-404 | *** |
| I-405 | * |
| I-406 | * |
| I-407 | *** |
| I-408 | *** |
| I-409 | * |
| I-410 | *** |
| I-411 | * |
| I-412 | *** |
| I-413 | *** |
| I-414 | * |
| I-415 | * |
| I-416 | ** |
| I-417 | ** |
| I-418 | *** |
| I-419 | * |
| I-420 | *** |
| I-421 | *** |
| I-422 | ** |
| I-423 | * |
| I-424 | * |
| I-425 | ** |
| I-426 | * |
| I-427 | * |
| I-428 | * |
| I-429 | * |
| I-430 | *** |
| I-431 | * |
| I-432 | *** |
| I-433 | * |
| I-434 | *** |
| I-435 | ** |
| I-436 | * |
| I-437 | * |
| I-438 | * |
| I-439 | * |
| I-440 | * |
| I-441 | * |
| I-442 | * |
| I-443 | ** |
| I-444 | * |
| I-445 | * |
| I-446 | * |
| I-447 | * |
| I-448 | * |
| I-449 | * |
| I-450 | * |
| I-453 | * |
| I-454 | * |
| I-456 | * |
| I-457 | * |
| I-459 | * |
| I-460 | * |
| I-461 | * |
| I-462 | * |
| I-463 | * |
| I-464 | * |
| I-466 | * |
| I-467 | * |
| I-468 | * |
| I-469 | ** |

### Example 15: BRET Assays

Background—The following assay can be used for determination of OXER1 activation in living HEK293 cells. Signalling functions of GPCRs are tightly regulated by endocytosis, the targeting of receptors to endosomes and their sorting to lysosomes or recycling to the plasma membrane. The early endosomes (EEs) trafficking assay (Namkung et al., Monitoring G protein-coupled receptor and β-arrestin trafficking in live cells using enhanced bystander BRET, Nat Commun 2016, 7: 12178) uses Rluc8-tagged OXER1 and Renilla GFP (rGFP) attached to the FYVE domain from human endofin/zinc finger FYVE domain-containing protein 16, which binds phosphatidylinositol 3-phosphate in EEs. Agonist stimulation of OXER1-Rluc8 leads to trafficking of the receptor to EEs, and ensuing increase of the donor concentration relative to the rGFP-FYVE acceptor anchored in the same cellular compartment, and thus results in an increase in BRET signal.

Plasmids—Human OXER1 VersaClone cDNA was obtained from R&D Systems. Rluc8 (A55T, C124A, S130A, K136R, A143M, M185V, M253L, and S287L variant of theRenilla renformis luciferase) gBlocks gene fragment (Integrated DNA Technologies, IA) was inserted with linker in frame at the C-terminus of OXER1. The FYVE domain from human endofin (residues Q739 to K806) attached in frame at the C terminus of a humanized Renilla GFP (rGFP), were synthesized as gBlocks gene fragments.

Bioluminescence Resonance Energy Transfer (BRET) Measurement—HEK293 cells were transfected with OXER1-Rluc8 and rGFP-FYVE. The following day, transiently transfected cells were seeded in 96-well white clear bottom microplates coated with poly-D-lysine and left in culture for 24 hours. Cells were washed once with Tyrode's buffer (140 mmol/L NaCl, 1 mmol/L CaCl₂, 2.7 mmol/L KCl, 0.49 mmol/L MgCl₂, 0.37 mmol/L NaH₂PO₄, 5.6 mmol/L glucose, 12 mmol/L NaHCO₃, and 25 mmol/L HEPES, pH 7.5) before performing assays in Tyrode's buffer. Test compounds were incubated with cells for 15 minutes at 37° C. before addition of 600 mmol/L of the OXER1 agonist 5-oxo-6E,8Z,1IZ,14Z-eicosatetraenoic acid (5-oxo-ETE) for 45 minutes at 37° C. The Rluc8 substrate coelenterazine 400A (Prolume, Lakeside, AZ) was added at a final concentration of 5 mol/L and BRET readings were collected using a Spark or Infinite M1000 microplate reader (Tecan, Morrisville, NC). BRET² readings between Rluc8 and rGFP were collected by sequential integration of the signals detected in the 370 to 450 nm (Rluc8) and 510 to 540 nm (rGFP) windows. The BRET signal was calculated as the ratio of light emitted by acceptor (rGFP) over the light emitted by donor (Rluc8). Ligand-promoted net BRET values were calculated by subtracting vehicle-induced net BRET from ligand-induced net BRET.

Results: Results are provided in Table 14 below for exemplary compounds. The symbol “***” indicates a IC₅₀ less than 100 nM. The symbol “**” indicates an IC₅₀ in the range of 100 nM to 300 nM. The symbol “*” indicates a IC₅₀ greater than 300 nM.

TABLE 14

| Compound No. | IC₅₀ |
|---|---|
| I-1 | ** |
| I-2 | *** |
| I-3 | *** |
| I-4 | * |
| I-5 | * |
| I-6 | * |
| I-7 | *** |
| I-8 | * |
| I-9 | *** |
| I-10 | *** |
| I-11 | ** |
| I-12 | ** |
| I-13 | *** |
| I-14 | ** |
| I-17 | * |
| I-19 | * |
| I-20 | * |
| I-21 | ** |
| I-22 | ** |
| I-23 | *** |
| I-24 | **** |
| I-39 | ** |
| I-40 | *** |
| I-44 | *** |
| I-45 | ** |
| I-47 | *** |
| I-48 | *** |
| I-59 | ** |
| I-60 | ** |
| I-68 | *** |
| I-73 | * |
| I-83 | ** |
| I-85 | *** |
| I-98 | *** |
| I-105 | ** |
| I-180 | *** |
| I-182 | ** |
| I-185 | ** |
| I-104 | ** |

While we have described a number of embodiments of this invention, it is apparent that our examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I: 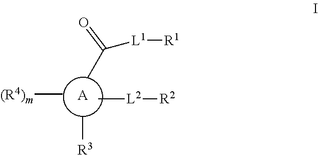
or an N-oxide, or a pharmaceutically acceptable salt thereof, wherein:
Ring A is an 8-10 membered aromatic or partially aromatic bicyclic ring having 1-4 heteroatoms independently selected from nitrogen, oxygen, and sulfur; a 5-6 membered monocyclic heteroaryl ring having 1-4 heteroatoms independently selected from nitrogen, oxygen, and sulfur; or phenyl;
L¹ is one of the following:
(a) a C₁₋₅ bivalent straight or branched, saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —C(F)₂—, —N(R)—, —S—, —S(O)—, or —S(O)₂—; or
(b) a covalent bond;
each R is independently hydrogen, or an optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur;
each -Cy- is independently an optionally substituted bivalent ring selected from a 4-7 membered saturated or partially unsaturated heterocyclylenyl having 1-2 heteroatoms independently selected from nitrogen, oxygen, and sulfur, phenylenyl, a 3-7 membered saturated or partially unsaturated carbocyclylenyl, or a 5-6 membered heteroarylenyl having 1-4 heteroatoms independently selected from nitrogen, oxygen, and sulfur;
L² is one of the following:
(a) a C₁₋₇ bivalent straight or branched saturated or unsaturated hydrocarbon chain wherein 1-2 methylene units of the chain are independently and optionally replaced with —O—, —C(O)—, —C(S)—, -Cy-, —C(R)₂—, —CH(R)—, —CH(OR)—, —CH(F)—, —C(F)₂—, —N(R)—, S—, —S(O)—, or —S(O)₂—; or
(b) a covalent bond;
R¹ is selected from (i) —C(O)OR, —C(O)N(R)S(O)₂R, —C(O)N(R)OR, —C(O)NR₂, —CN, —OH, and hydrogen; (ii) a 5-6 membered partially unsaturated oxo-heterocyclyl containing 1-4 heteroatoms independently selected from nitrogen, oxygen, and sulfur; which is substituted with n instances of R⁵; and (iii) a 5-6 membered monocyclic heteroaryl ring having 1-4 heteroatoms independently selected from nitrogen, oxygen, and sulfur; which is substituted with n instances of R⁵;
R² is one of the following:
(a) phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered aromatic or partially aromatic bicyclic ring having 1-4 heteroatoms independently selected from nitrogen, oxygen, and sulfur; each of which is substituted with p instances of R⁶; or
(b) C₁₋₇ aliphatic, —C≡CR, —C(O)OR, or —C(O)R; each of which is substituted with p instances of R⁶; or
(c) hydrogen;
R³ is one of the following:
(a) a C₁₋₆ aliphatic group optionally substituted with one or more —OH or —N(R)₂;
(b) —CD₃;
(c) hydrogen; or
(d) absent;
each instance of R⁴ is independently hydrogen, deuterium, R^{z}, —C≡CR, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂; or
two R⁴ groups are optionally taken together to form ═O; or
two R⁴ groups are optionally taken together with their intervening atoms to form an optionally substituted 5-8 membered saturated, partially unsaturated, or aryl fused ring having 0-3 heteroatoms independently selected from nitrogen, oxygen and sulfur;
each instance of R⁵, and R⁶ is independently hydrogen, deuterium, R^{z}, halogen, —CN, —NO₂, —OR, —OCF₃, —SR, —NR₂, —S(O)₂R, —S(O)₂NR₂, —S(O)R, —S(O)NR₂, —CF₂R, —CF₃, —CR₂(CN), —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —Si(OR)R₂, —SiR₃, —P(O)(R)NR₂, —P(O)(R)OR or —P(O)R₂; or
two R⁵ groups are optionally taken together to form ═O;
two R⁶ groups are optionally taken together to form ═O;
two R⁵ groups are optionally taken together with their intervening atoms to form an optionally substituted 5-8 membered saturated, partially unsaturated, or aryl fused ring having 0-3 heteroatoms independently selected from nitrogen, oxygen and sulfur; or
two R⁶ groups are optionally taken together with their intervening atoms to form an optionally substituted 5-8 membered saturated, partially unsaturated, or aryl fused ring having 0-3 heteroatoms independently selected from nitrogen, oxygen and sulfur;
each instance of R^{z} is independently selected from an optionally substituted group selected from C₁₋₆ aliphatic; phenyl; a 4-7 membered saturated or partially unsaturated heterocyclic ring having 1-2 heteroatoms independently selected from nitrogen, oxygen, and sulfur; and a 5-6 membered heteroaryl ring having 1-4 heteroatoms independently selected from nitrogen, oxygen, and sulfur;
m is 0, 1, 2, 3, or 4;
n is 0, 1, 2, 3, or 4; and
p is 0, 1, 2, 3, or 4;
provided that if L¹-R¹ is 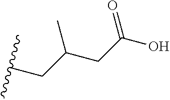 [SMILES: *CC(C)CC(=O)O]
and ring A with its R³ and R⁴ substituents is 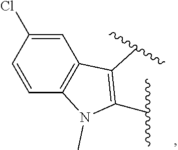 [SMILES: *c1c(*)n(C)c2ccc(Cl)cc12]
then L²-R² is not n-hexyl, 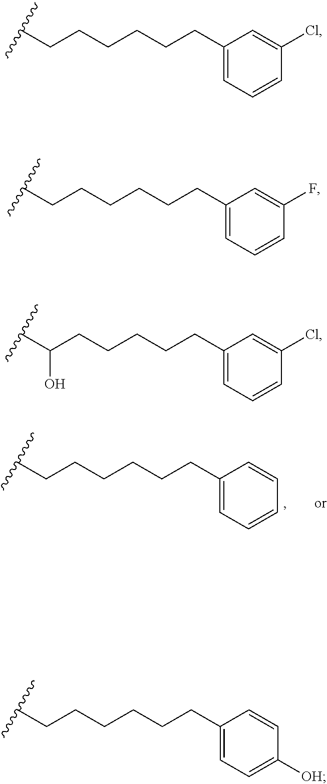 [SMILES: *CCCCCCc1cccc(Cl)c1 ; *CCCCCCc1cccc(F)c1 ; *C(O)CCCCCc1cccc(Cl)c1 ; *CCCCCCc1ccccc1 ; *CCCCCCc1ccc(O)cc1]
and
provided that if L¹-R¹ is 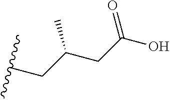 [SMILES: *C[C@H](C)CC(=O)O]
and ring A with its R³ and R⁴ substituents is 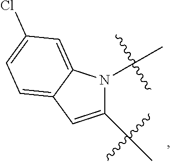 [SMILES: *c1cc2ccc(Cl)cc2n1*]
then L²-R² is not n-hexyl.

2. The compound of claim 1, wherein Ring A substituted with (R⁴)ₘ, R³—C(O)-L¹-R¹, and L²-R² is

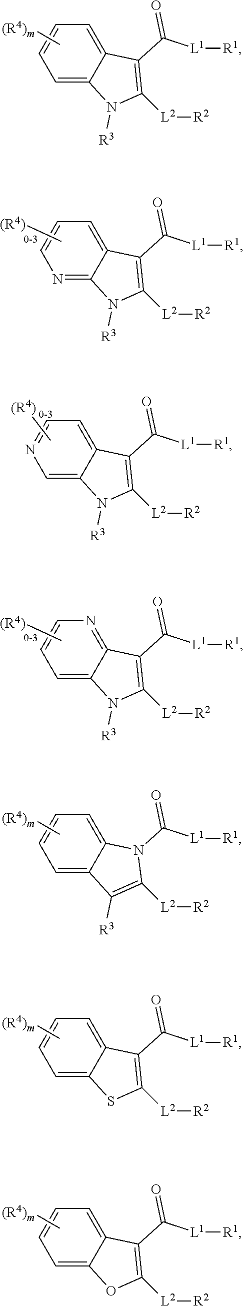

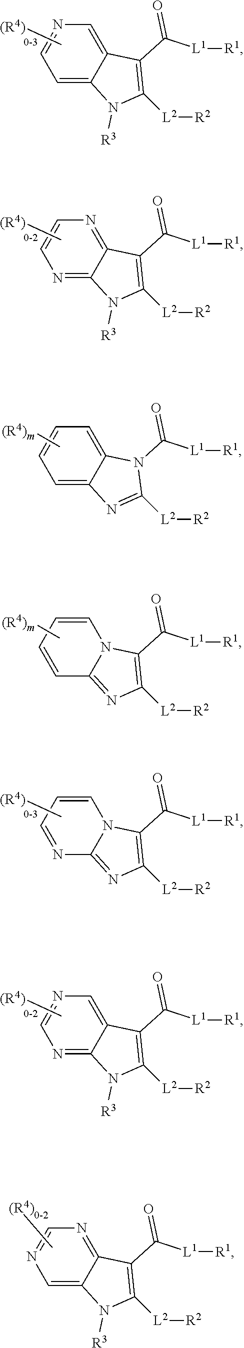

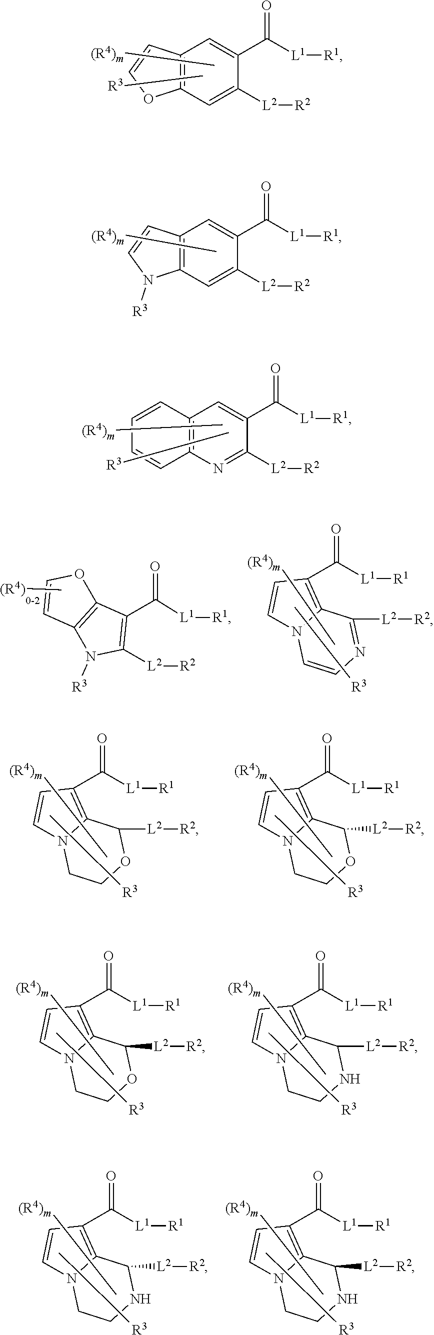

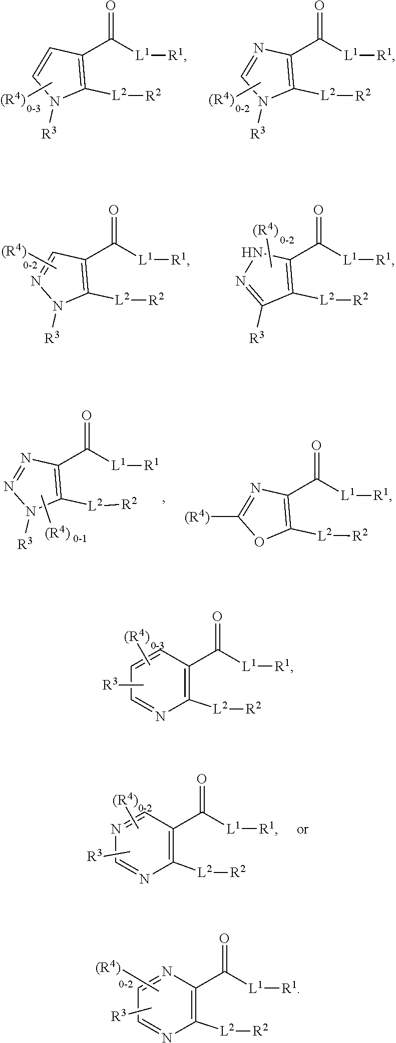

3. The compound of claim 1 or 2, wherein the compound is of any one of formulae I-a-1, I-a-2, I-a-3, or I-a-4: 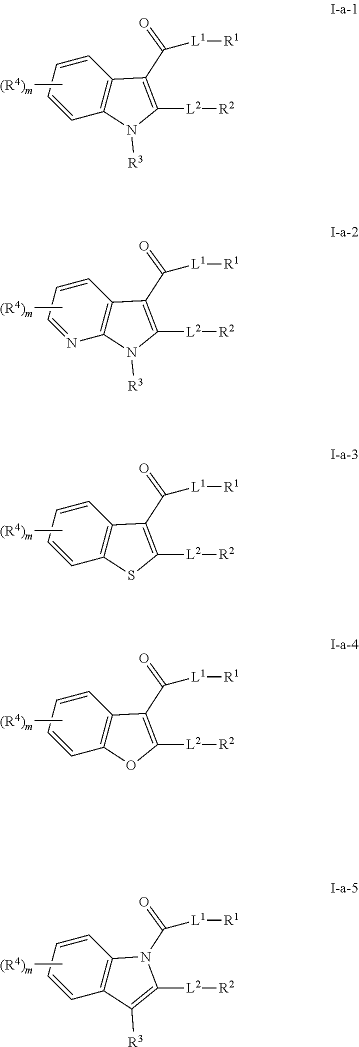
or an N-oxide, or a pharmaceutically acceptable salt thereof.

4. The compound of claim 3, wherein the compound is of any one of formulae I-a-1, or I-a-2: 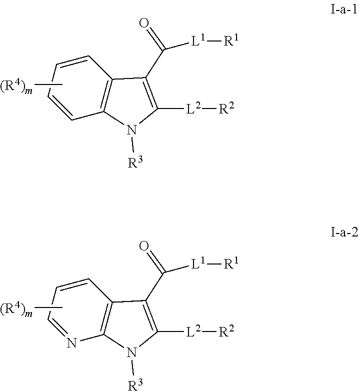
or an N-oxide, or a pharmaceutically acceptable salt thereof.

5. The compound of claim 4, wherein the compound is of formula I-a-2: 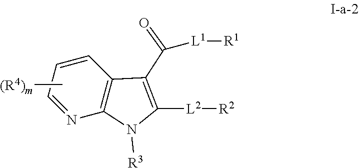
or an N-oxide, or a pharmaceutically acceptable salt thereof.

6. The compound of any one of claims 1-5, wherein L¹ is a C₁₋₅ bivalent straight or branched, saturated hydrocarbon chain.

7. The compound of claim 6, wherein L¹ is

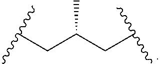

8. The compound of any one of claims 1-5, wherein L¹ is a C₁₋₅ bivalent straight or branched, saturated hydrocarbon chain wherein 1 methylene unit of the chain is replaced with -Cy-, wherein Cy is a bivalent ring selected from a 4-7 membered saturated heterocyclylenyl having 1-2 heteroatoms independently selected from nitrogen, oxygen, and sulfur.

9. The compound of claim 8, wherein L¹ is

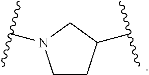 [SMILES: *C1CCN(*)C1]

10. The compound of any one of claims 1-5, wherein L¹ is

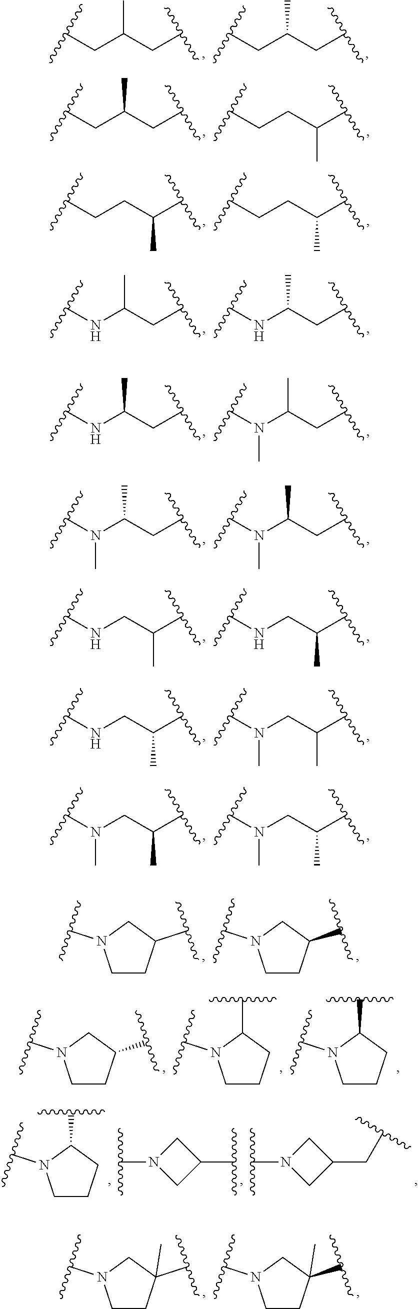

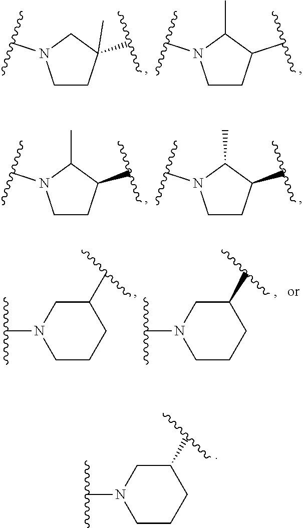

11. The compound of claim 10, wherein L¹ and R¹ is

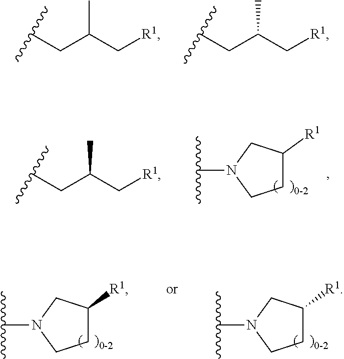

12. The compound of claim 11, wherein L¹ and R¹ is

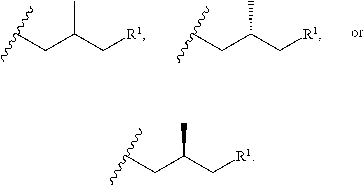

13. The compound of claim 11, wherein L¹ and R¹ is

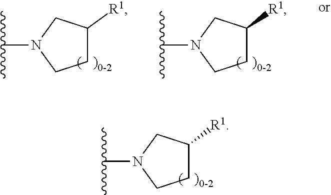

14. The compound of any one of claims 1-13, wherein L² is a C₁₋₇ bivalent straight or branched saturated hydrocarbon chain wherein 1 methylene unit of the chain is replaced with —O—.

15. The compound of any one of claims 1-13, wherein L² is —(CH₂)₃₋₄—O—(CH₂)₁₋₂—.

16. The compound of any one of claims 1-13, wherein L² is —(CH₂)₃₋₄—O—(C(H)(CH₃))—.

17. The compound of any one of claims 1-13, wherein L² is a C₁₋₇ bivalent straight or branched unsaturated hydrocarbon chain.

18. The compound of any one of claims 1-13, wherein L² is a C₁ bivalent straight unsaturated hydrocarbon chain.

19. The compound of any one of claims 1-13, wherein L² is

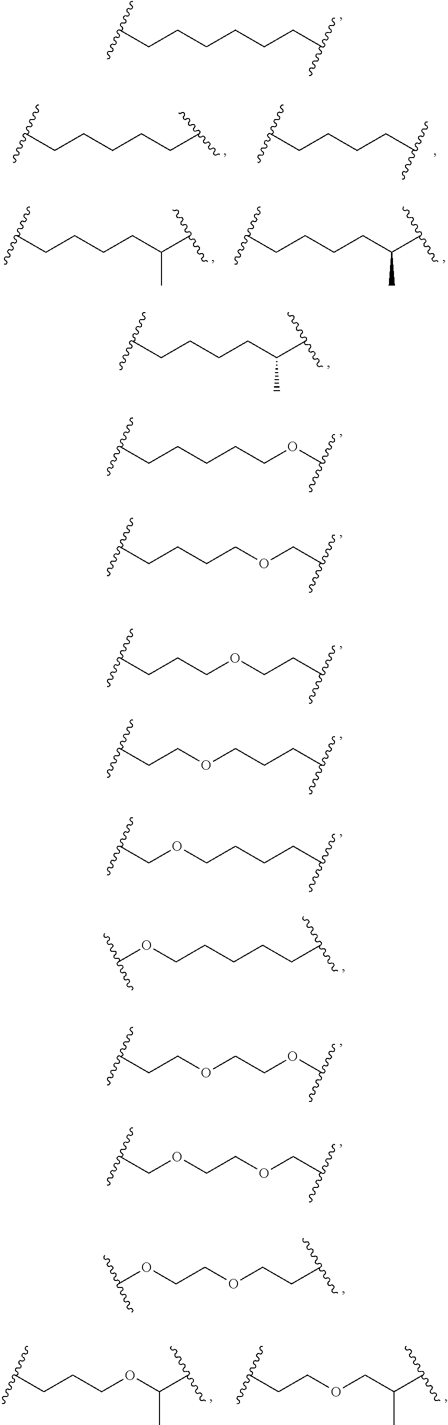

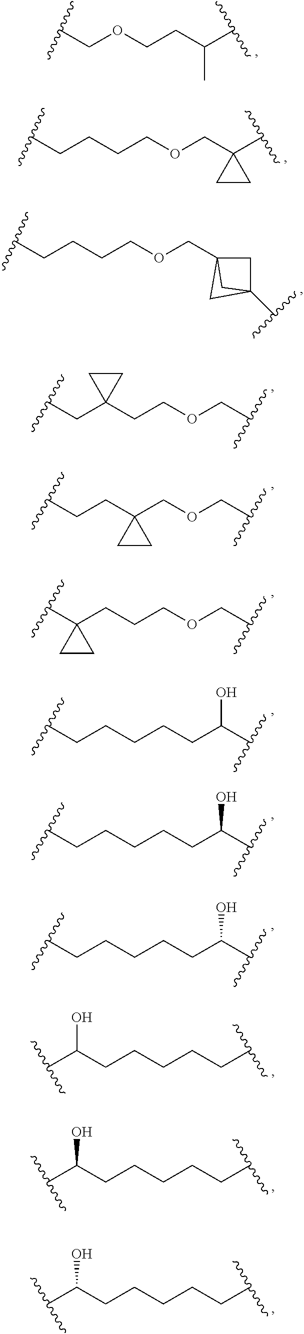

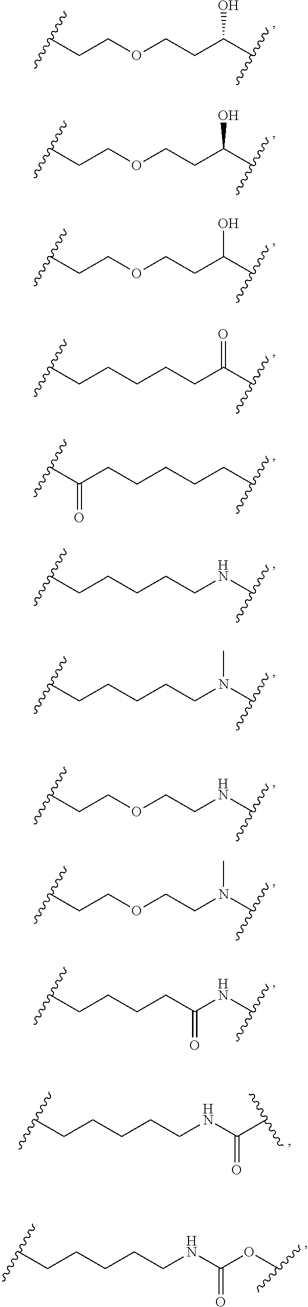

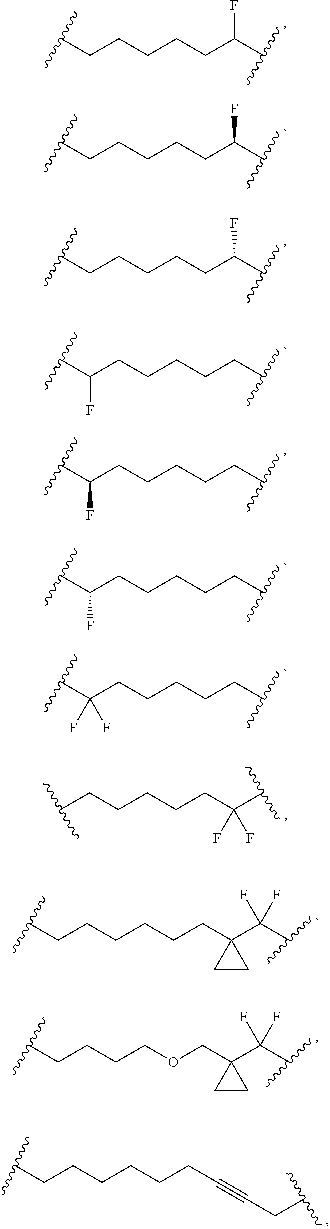

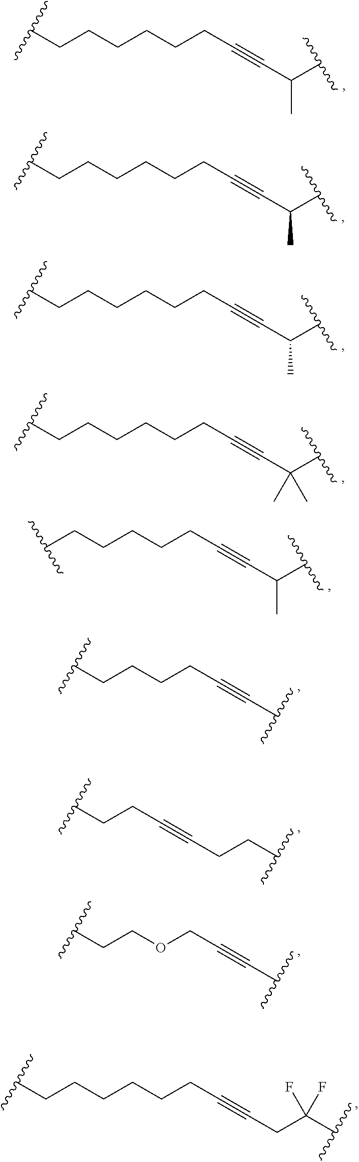

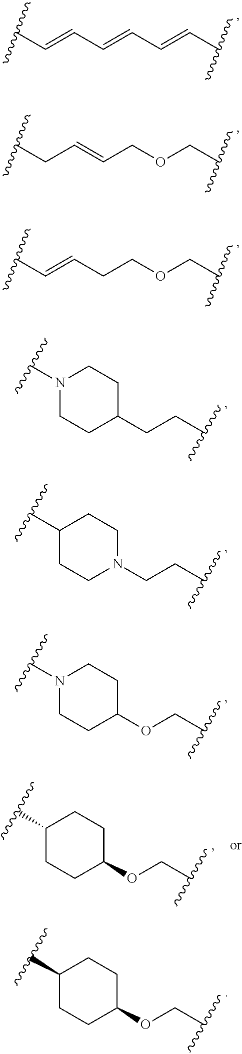

20. The compound of claim 19, wherein L² and R² is

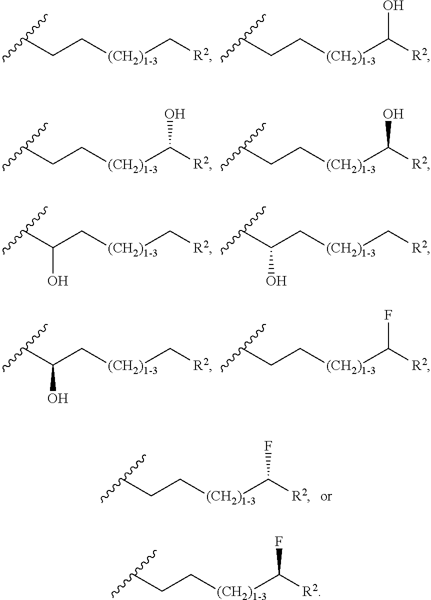

21. The compound of claim 19, wherein L² and R² is

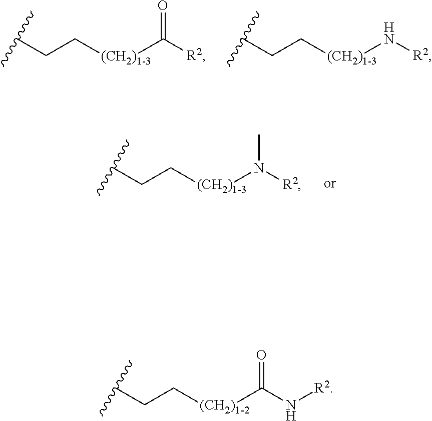

22. The compound of claim 19, wherein L² and R² is

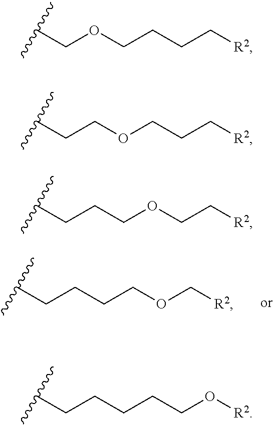

23. The compound of claim 19, wherein L² and R² is

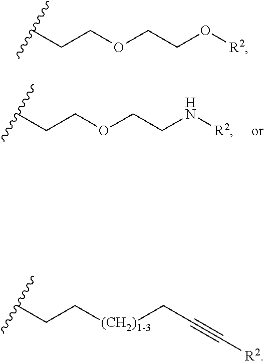

24. The compound of any one of claims 1-23, wherein wherein R¹ is

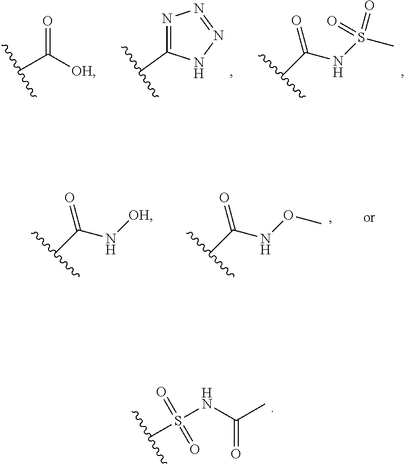

25. The compound of claim 24, wherein R¹ is

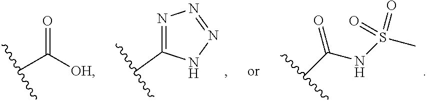

26. The compound of claim 24, wherein R¹ is OH

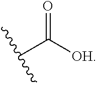 [SMILES: *C(=O)O]

27. The compound of any one of claims 1-26 wherein R² is

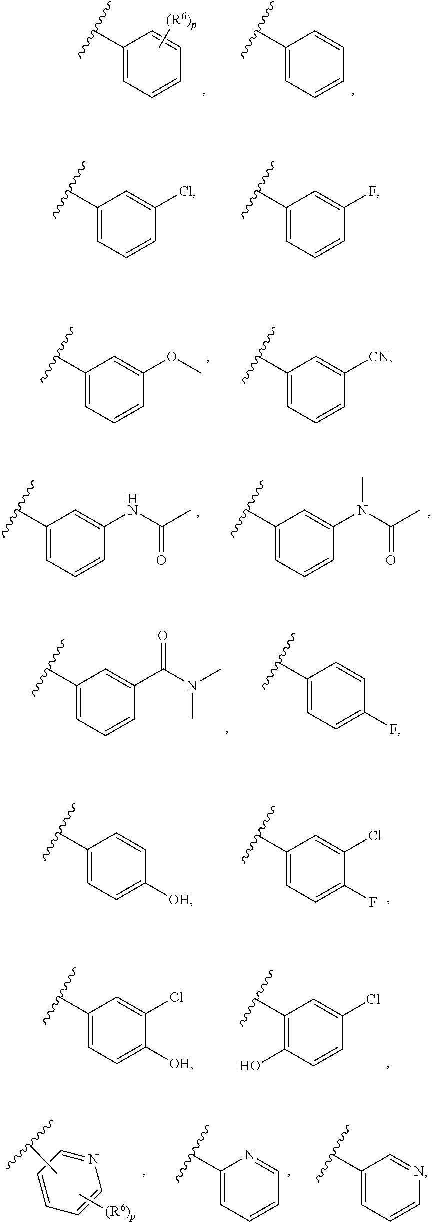

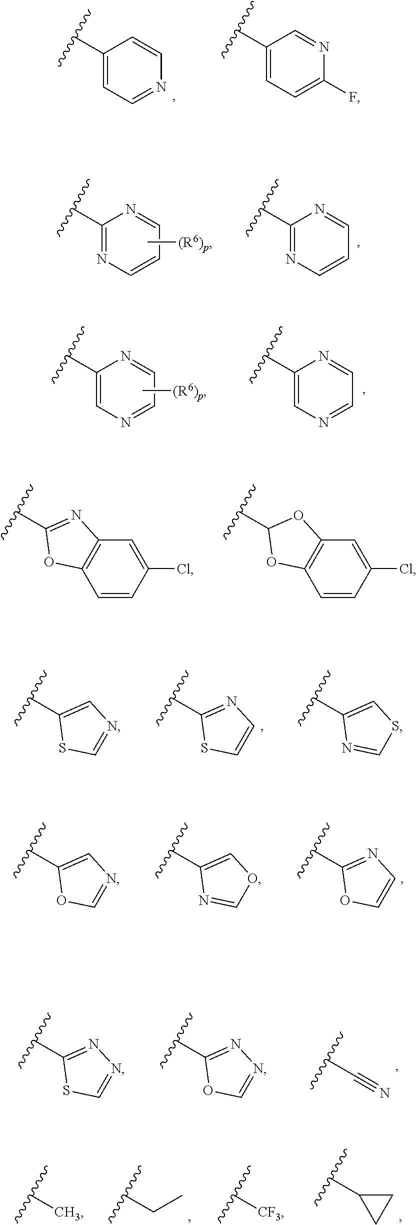

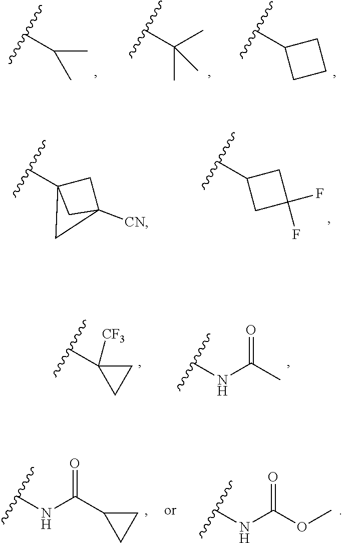

28. The compound of claim 27, wherein R² is

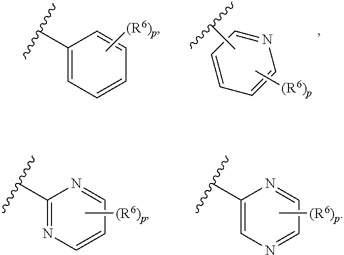

29. The compound of claim 27, wherein R² is

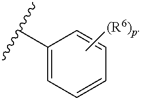

30. The compound of claim 27, wherein R² is

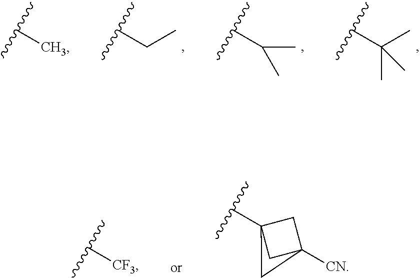

31. The compound of any one of claims 1-26, wherein R² is —C≡CR.

32. The compound of any one of claims 1-26, wherein R² is —C≡C—(3-7 membered saturated monocyclic carbocylic ring substituted with 1 or 2 halo groups).

33. The compound of any one of claims 1-26, wherein R² is —C≡C—(cyclobutyl substituted with 1 or 2 halo groups).

34. The compound of any one of claims 1-33, wherein R³ is hydrogen, —CH₃, —CD₃, 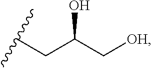 [SMILES: *C[C@@H](O)CO]
or absent.

35. The compound of claim 34, wherein R³ is —CH₃.

36. The compound of any one of claims 1-35, wherein R⁴ is hydrogen, —C₁, —CF₃, —OCH₃, —OCF₃, —CN, or

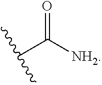 [SMILES: *C(N)=O]

37. The compound of claim 36, wherein R⁴ is —Cl, or —CN.

38. The compound of claim 37, wherein R⁴ is —CN.

39. The compound of any one of claims 1-38, wherein m is 1.

40. The compound of any one of claims 1-35, wherein m is 0.

41. The compound of claim 1, wherein the compound is of any one of formulae I-i-1, I-i-2, I-i-3, I-i-4, I-i-5, or I-i-6: 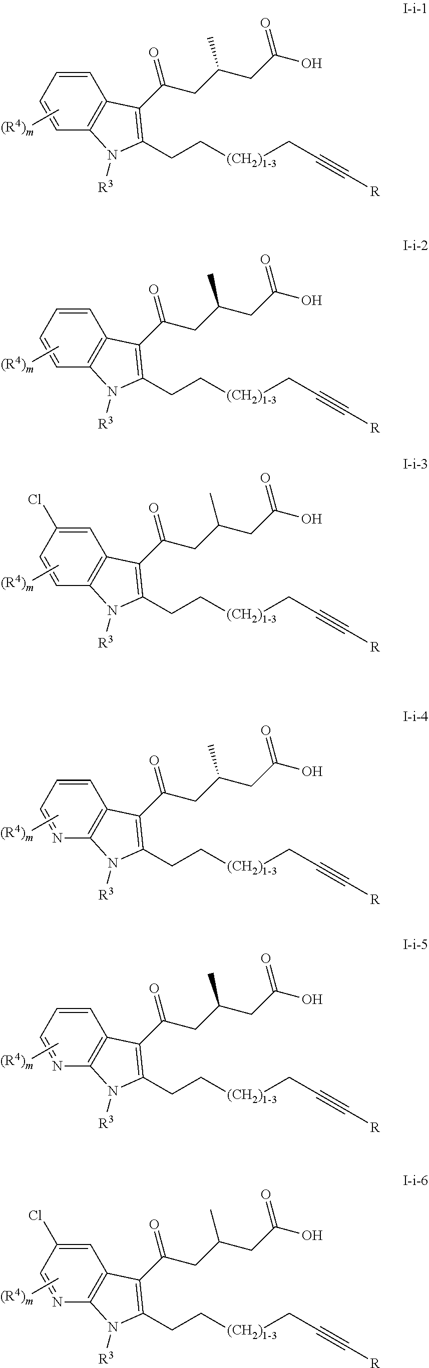
or an N-oxide, or a pharmaceutically acceptable salt thereof.

42. The compound of claim 41, wherein the compound is of any one of formulae I-i-4, I-i-5, or I-i-6: 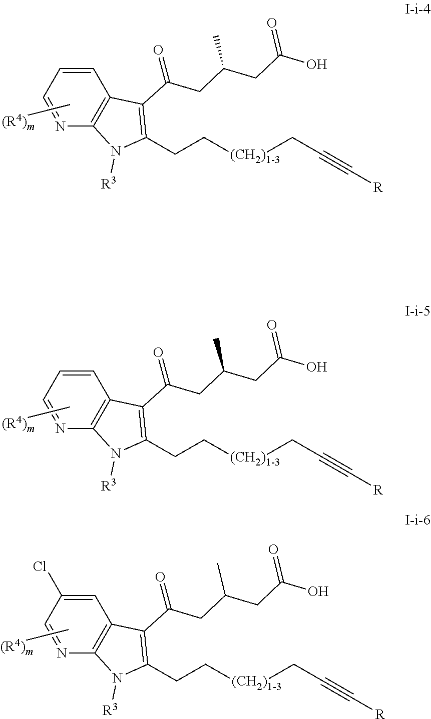
or an N-oxide, or a pharmaceutically acceptable salt thereof.

43. The compound of claim 1, wherein the compound is of any one of formulae I-j-1, I-j-2, I-j-3, I-j-4, I-j-5, or I-j-6: 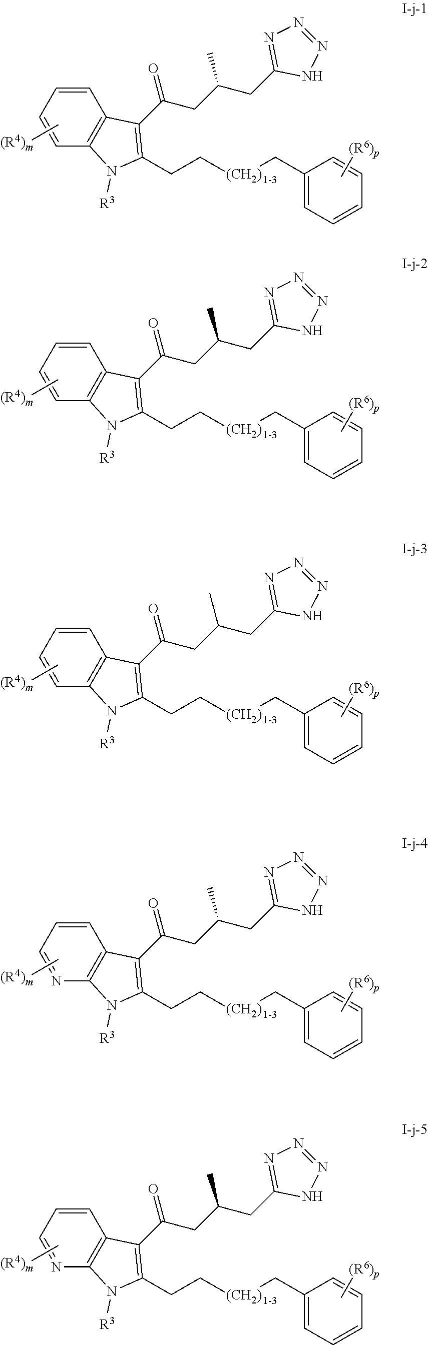 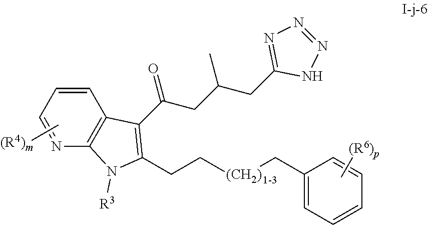
or an N-oxide, or a pharmaceutically acceptable salt thereof.

44. The compound of claim 43, wherein the compound is of any one of formulae I-j-4, I-j-5, or I-j-6: 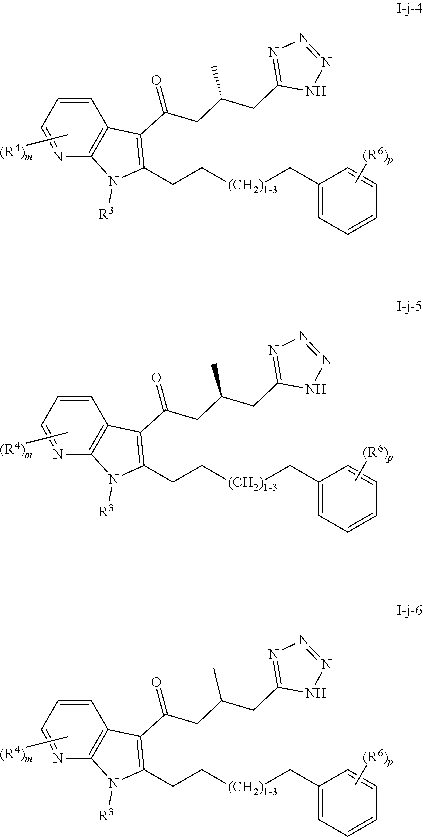
or an N-oxide, or a pharmaceutically acceptable salt thereof.

45. The compound of claim 1, wherein the compound is of any one of formulae I-j-7, I-j-8, I-j-9, I-j-10, I-j-11, or I-j-12: 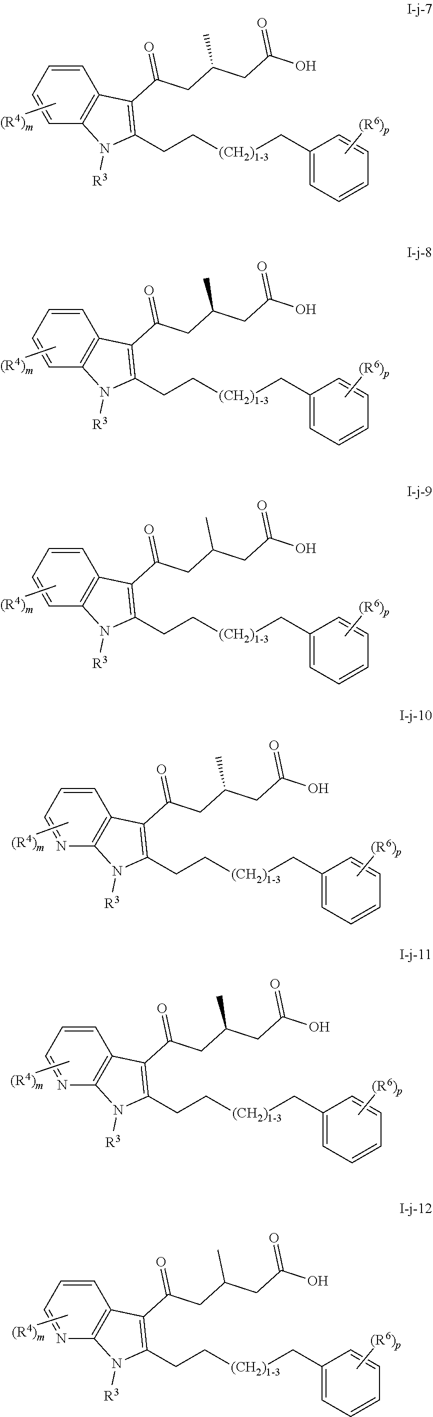
or an N-oxide, or a pharmaceutically acceptable salt thereof.

46. The compound of claim 45, wherein the compound is of any one of formulae I-j-10, I-j-11, or I-j-12: 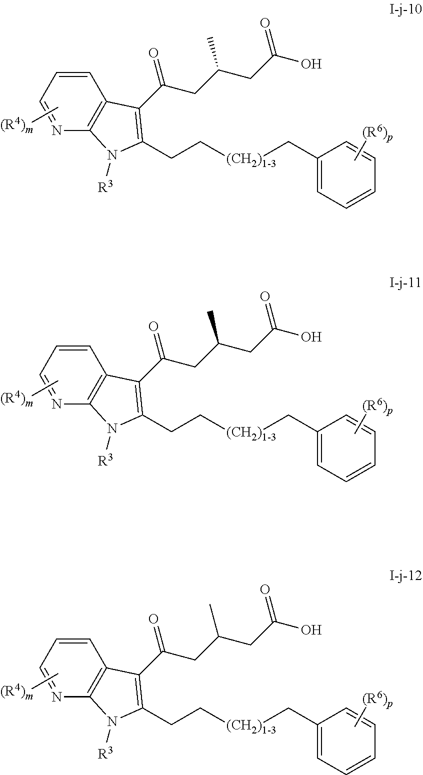
or an N-oxide, or a pharmaceutically acceptable salt thereof.

47. The compound of claim 1, wherein the compound is of any one of formulae I-k-1, I-k-2, I-k-3, I-k-4, I-k-5, or I-k-6: 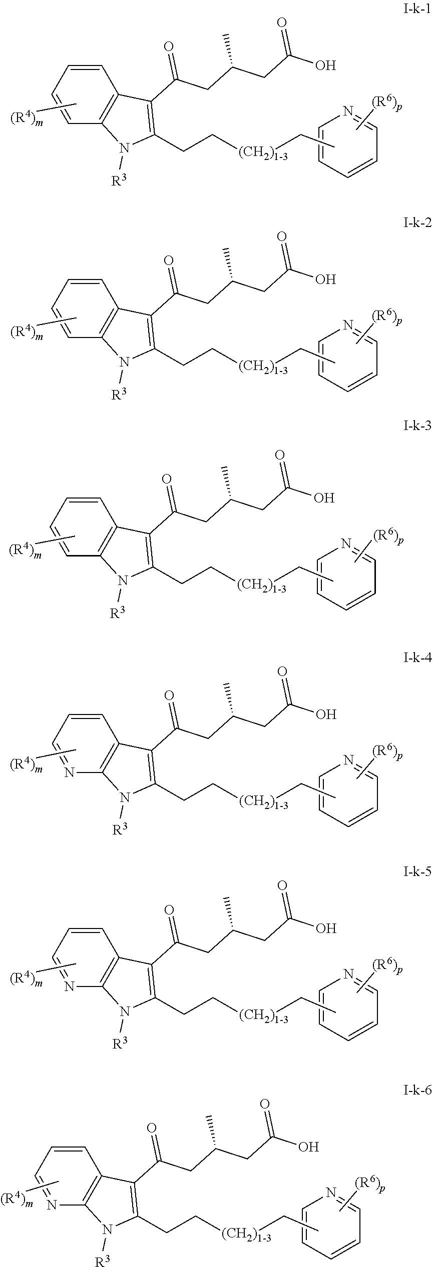
or an N-oxide, or a pharmaceutically acceptable salt thereof.

48. The compound of claim 47, wherein the compound is of any one of formulae I-k-4, I-k-5, or I-k-6: 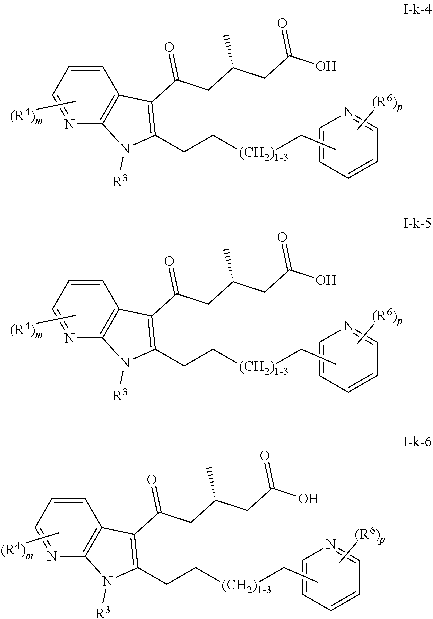
or an N-oxide, or a pharmaceutically acceptable salt thereof.

49. The compound of claim 1, wherein the compound is of any one of formulae I-q-1, I-q-2, I-q-3, I-q-4, I-q-5, or I-q-6: 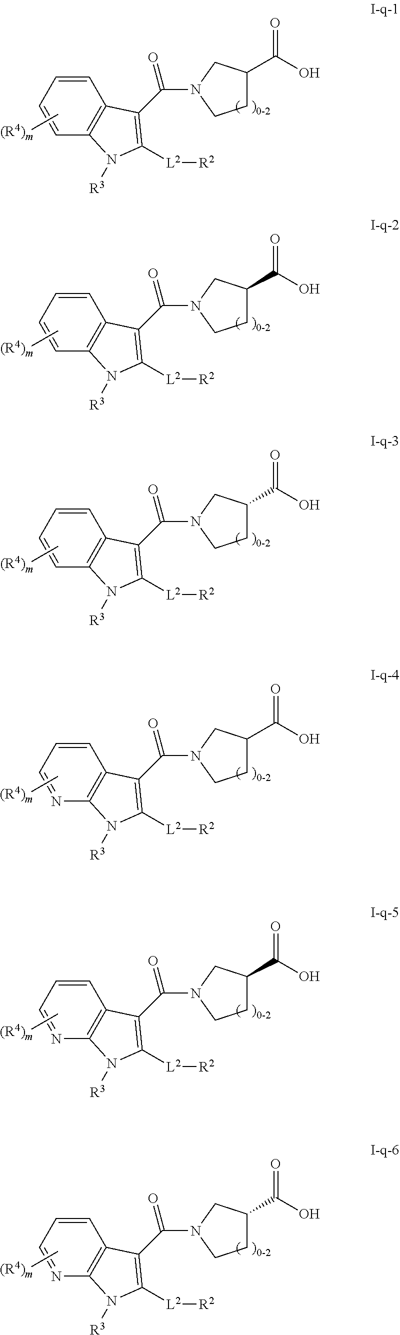
or an N-oxide, or a pharmaceutically acceptable salt thereof.

50. The compound of claim 49, wherein the compound is of any one of formulae I-q-4, I-q-5, or I-q-6: 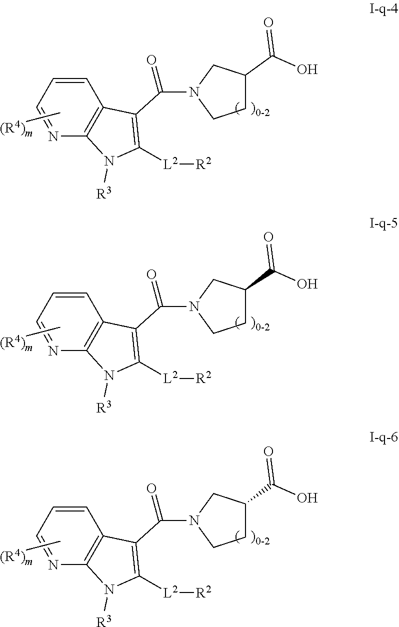
or an N-oxide, or a pharmaceutically acceptable salt thereof.

51. The compound of claim 1, wherein the compound is of any one of formulae I-q-7, I-q-8, I-q-9, or I-q-10: 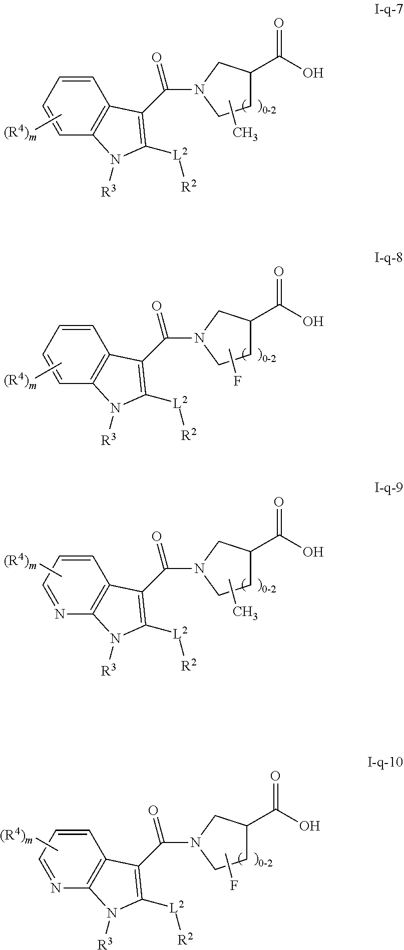
or a pharmaceutically acceptable salt thereof.

52. The compound of claim 1, wherein the compound is of formulae I-q-11: 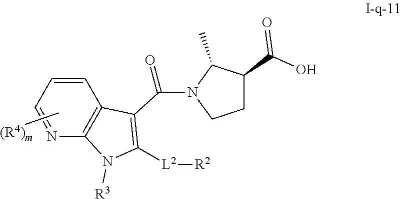
or a pharmaceutically acceptable salt thereof.

53. The compound of claim 1, wherein the compound is of any one of formulae I-r-1, I-r-2, I-r-3, I-r-4, I-r-5_, or I-r-6: 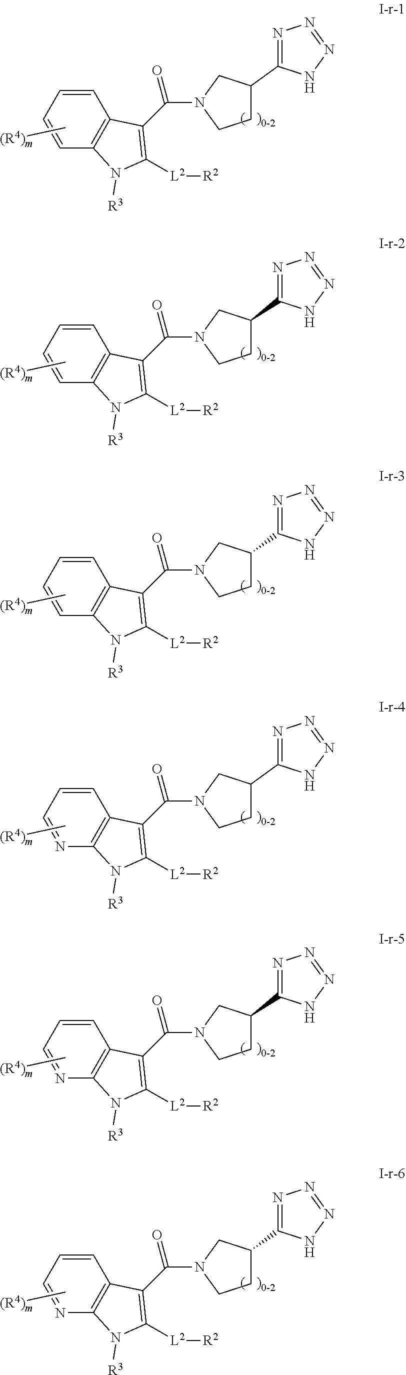
or an N-oxide, or a pharmaceutically acceptable salt thereof.

54. The compound of claim 53, wherein the compound is of any one of formulae I-r-4, I-r-5, or I-r-6: 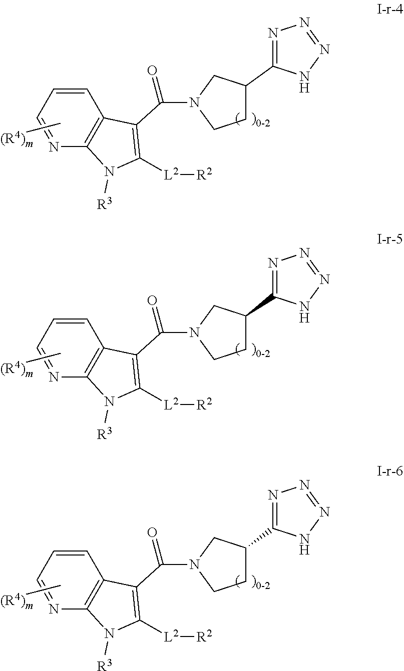
or an N-oxide, or a pharmaceutically acceptable salt thereof.

55. The compound of claim 1, wherein the compound is of any one of formulae I-r-7, I-r-8, I-r-9: 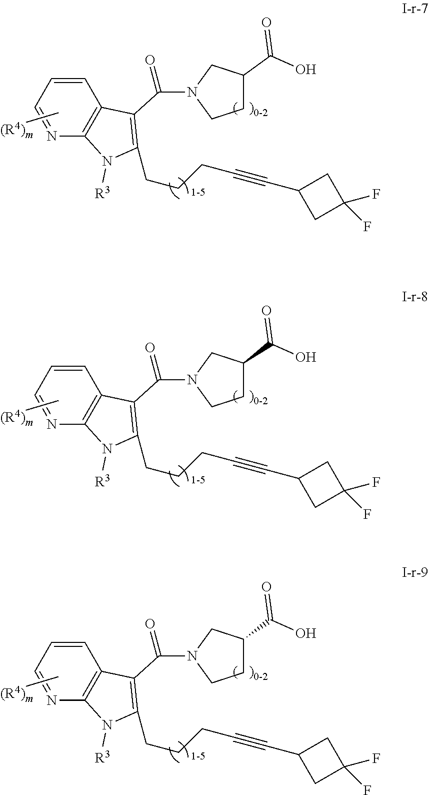
or a pharmaceutically acceptable salt thereof.

56. The compound of claim 1, wherein the compound is of formulae I-r-10: 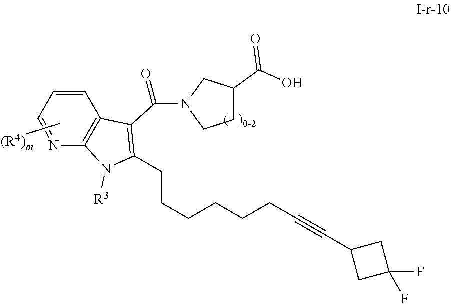
or a pharmaceutically acceptable salt thereof.

57. The compound of claim 1, wherein the compound is of any one of formulae I-f-1, I-f-2, I-f-3, I-f-4, I-f-5, or I-f-6: 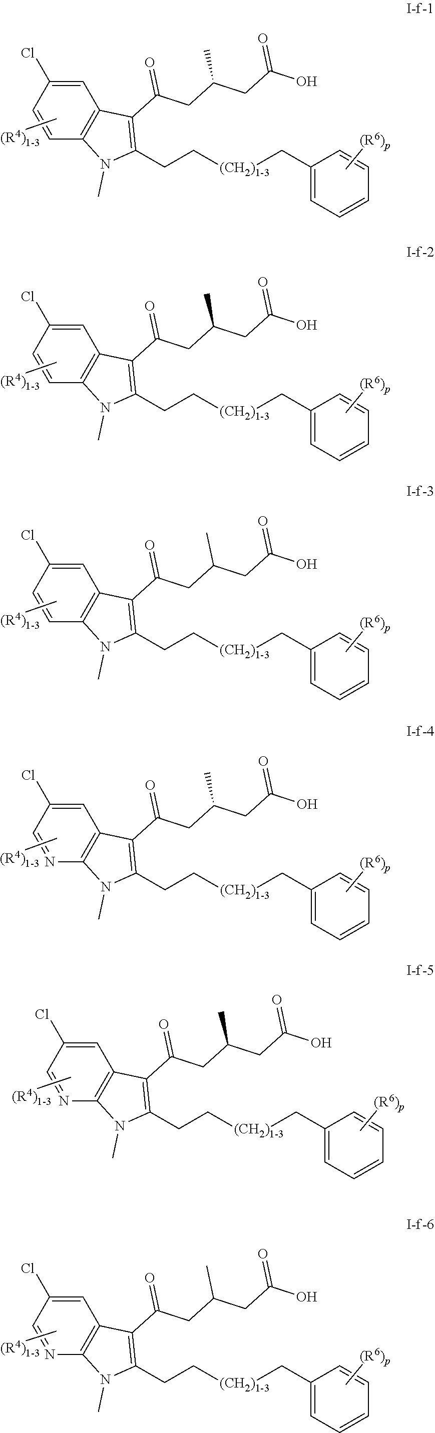
or an N-oxide, or a pharmaceutically acceptable salt thereof.

58. The compound of claim 57, wherein the compound is of any one of formulae I-f-4, I-f-5, or I-f-6: 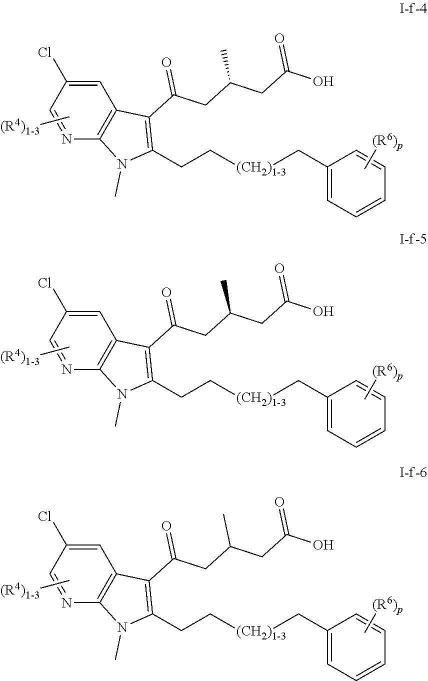
or an N-oxide, or a pharmaceutically acceptable salt thereof.

59. The compound of claim 1, wherein the compound is of any one of formulae I-g-1, I-g-2, I-g-3, I-g-4, I-g-5, or I-g-6: 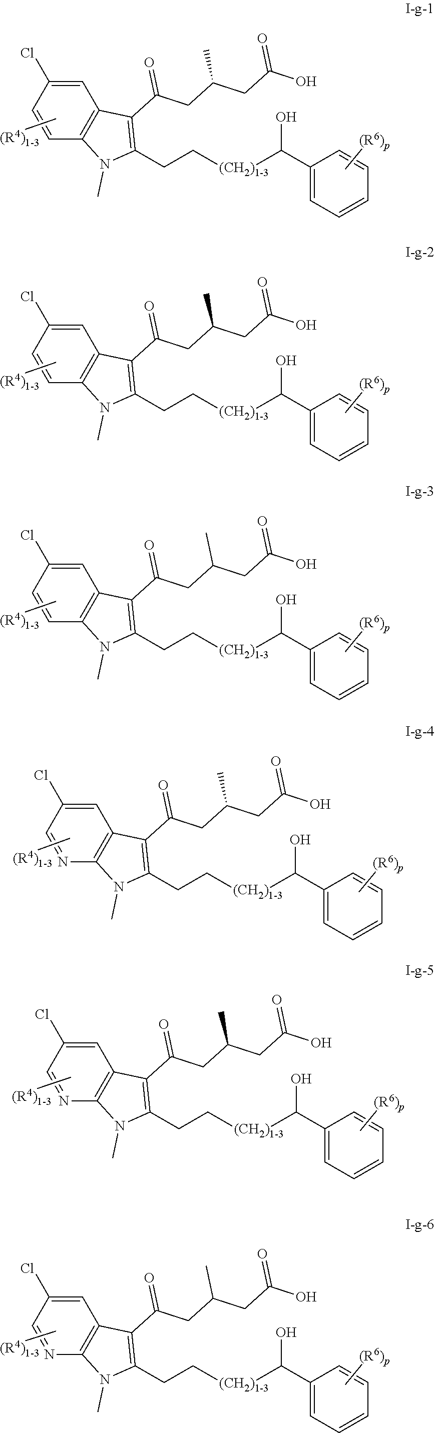
or an N-oxide, or a pharmaceutically acceptable salt thereof.

60. The compound of claim 59, wherein the compound is of any one of formulae I-g-4, I-g-5, or I-g-6: 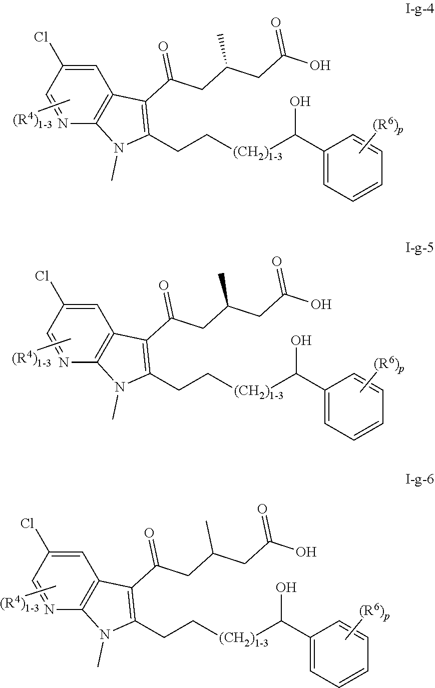
or an N-oxide, or a pharmaceutically acceptable salt thereof.

61. The compound of claim 1, wherein the compound is of any one of formulae I-h-1, I-h-2, I-h-3, I-h-4, I-h-5, or I-h-6: 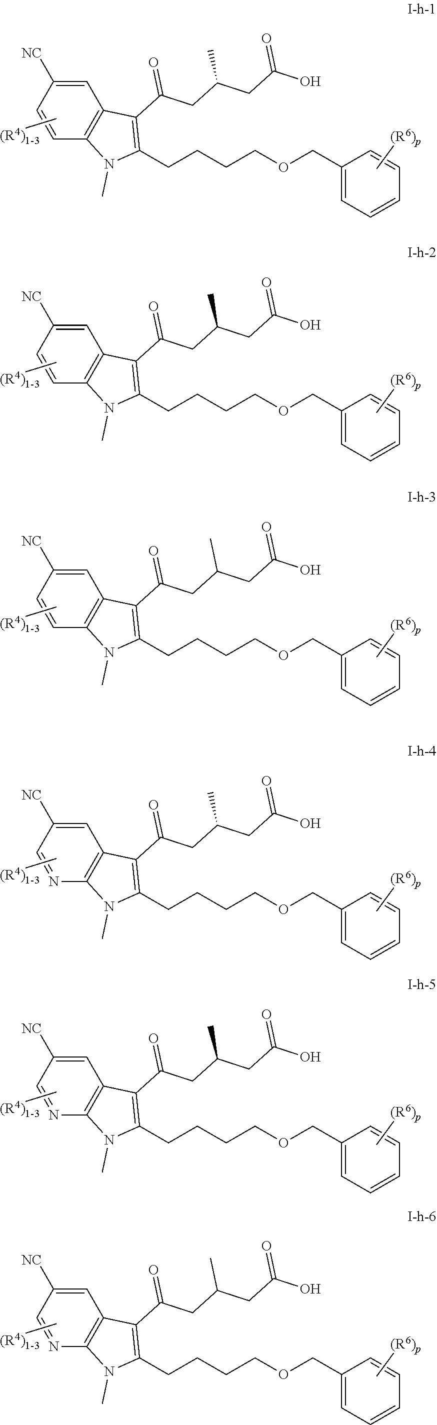
or an N-oxide, or a pharmaceutically acceptable salt thereof.

62. The compound of claim 61, wherein the compound is of any one of formulae I-h-4, I-h-5, or I-h-6: 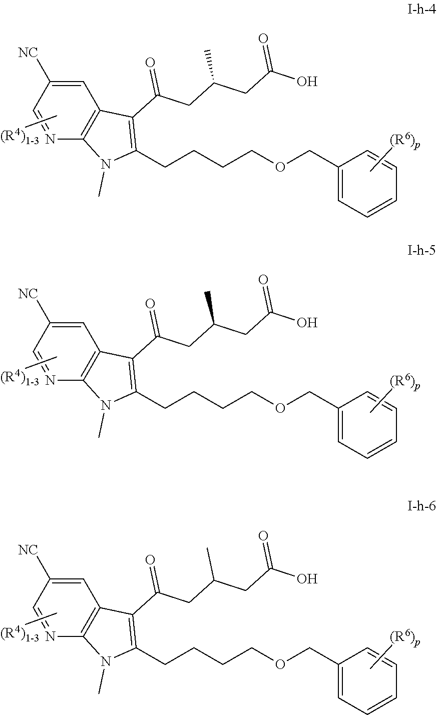
or an N-oxide, or a pharmaceutically acceptable salt thereof.

63. The compound of any one of claims 57-62, wherein R⁴ and R⁶ each represent independently for each occurrence hydrogen, halogen, or C₁₋₆ aliphatic.

64. The compound of claim 1, wherein the compound is selected from those depicted in Table 1, or an N-oxide, or a pharmaceutically acceptable salt thereof.

65. A pharmaceutical composition comprising a compound according to any one of claims 1-64, or an N-oxide, or a pharmaceutically acceptable salt thereof, and a pharmaceutically acceptable carrier, adjuvant, or vehicle.

66. The compound of any one of claims 1-64, or the pharmaceutical composition of claim 62, for use as a medicament.

67. A method of antagonizing OXER1 in a biological sample comprising contacting the sample with the compound of any one of claims 1-64, or an N-oxide, or a pharmaceutically acceptable salt thereof, or the pharmaceutical composition of claim 65.

68. A method of treating a OXER1-mediated disorder, disease, or condition in a patient comprising administering to said patient in need thereof the compound of any one of claims 1-64, or an N-oxide, or a pharmaceutically acceptable salt thereof, or the pharmaceutical composition of claim 65.

69. The method of claim 68, wherein the disorder, disease, or condition is a disorder, disease, or condition associated with inflammation, or cancer.

70. The method of claim 69, wherein the disorder, disease, or condition is an inflammation-driven disease, comprising skin, respiratory and gastro-intestinal diseases.

71. The method of claim 70, wherein the disease, disease, or condition is asthma, severe eosinophilic asthma, late phase of inflammatory asthma, chronic obstructive pulmonary disease (COPD), hypereosinophilic syndrome (HES), nasal polyposis, allergic inflammation, allergic rhinitis, atopic dermatitis, chronic spontaneous urticaria, psoriasis, acne, idiopathic pulmonary fibrosis, eosinophilic gastritis, eosinophilic esophagitis (EoE), eosinophilic gastroenteritis, arthritis, atherosclerosis, or acute myocardial infarction.

72. The method of claim 71, wherein the disease, disease, or condition is asthma.

73. The method of claim 71 or 72, further comprising concomitant administration of a second agent to the subject, wherein the second agent is an analgesic, anti-inflammatory agent, or anti-allergy agent.

74. The method of claim 73, wherein the second agent is an NSAID, a bronchodilator, a glucocorticoid, a cysLT1 antagonist, or a leukotriene modifier.

75. The method of claim 73, wherein the compound and the second agent are administered at the same time.

76. The method of claim 73, wherein the compound and the second agent are administered sequentially.

77. The method of claim 73, wherein the disorder, disease, or condition is cancer.

78. The method of claim 77, wherein the cancer is prostate cancer, triple-negative breast cancer, or ER⁻ breast cancer.
